SEQUENCE LISTING (8 sequences)

SEQ ID NO: 1 - protein, 5 aa, Artificial
  Chemically synthesized
NYGVH

SEQ ID NO: 2 - protein, 16 aa, Artificial
  Chemically synthesized
VIWSGGNTDYNTPFTS

SEQ ID NO: 3 - protein, 11 aa, Artificial
  Chemically synthesized
ALTYYDYEFAY

SEQ ID NO: 4 - protein, 11 aa, Artificial
  Chemically synthesized
RASQSIGTNIH

SEQ ID NO: 5 - protein, 7 aa, Artificial
  Chemically synthesized
YASESIS

SEQ ID NO: 6 - protein, 9 aa, Artificial
  Chemically synthesized
QQNNNWPTT

SEQ ID NO: 7 - protein, 168 aa, Artificial
  Chemically synthesized
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKA

SEQ ID NO: 8 - protein, 168 aa, Artificial; identical to SEQ ID NO: 7
  Chemically synthesized

(19) United States
(12) Patent Application Publication
(10) Pub. No.: US 2011/0142822 A1
(43) Pub. Date: Jun. 16, 2011

(54) Crystal of egfr extracellular domain and cetuximab fab fragment, and uses thereof

(76) Inventors: Paul H. Kussie, New York, NY (US); Kathryn M. Ferguson, Media, PA (US)

(21) Appl. No.: 11/629,415

(22) PCT Filed: Jun. 14, 2005
(86) PCT No.: PCT/US05/20969

§ 371 Date: Nov. 14, 2008

Related U.S. Application Data
(60) Provisional application No. 60/579,843, filed on Jun. 14, 2004.
(60) Provisional application No. 60/634,363, filed on Dec. 8, 2004.

(51) Int. Cl. A61K 39/395; C07K 16/00; C40B 30/02; G01N 33/53; A61P 35/00; G06F 19/00

(52) U.S. Cl. 424/133.1; 530/391.1; 506/8; 436/501; 530/387.3; 702/19

(57) ABSTRACT

The present invention relates to co-crystals of cetuximab Fab in a complex with extracellular domain of EGFR, and structure coordinates obtained from such crystal. Such coordinates are useful for identifying mimetics that bind to the extracellular domain of EGFR. Such mimetics may for example inhibit binding of ligand to EGFR, inhibit activation of EGFR, and/or reduce proliferation of tumor cells.

### FIELD OF THE INVENTION

The present invention relates to co-crystals of cetuximab Fab in a complex with extracellular domain of EGFR, and structure coordinates obtained from such crystal. Such coordinates are useful for identifying mimetics, preferably EGFR antagonists, that bind to the extracellular domain of EGFR. Such mimetics may for example inhibit binding of ligand to EGFR, inhibit activation of EGFR, and/or reduce proliferation of tumor cells.

### BACKGROUND OF THE INVENTION

Although normal cells proliferate by the highly controlled activation of growth factor receptor tyrosine kinases (“RTKs”) by their respective ligands, cancer cells also proliferate by the activation of growth factor receptors, but lose the careful control of normal proliferation. The loss of control may be caused by numerous factors, such as the overexpression of growth factors and/or receptors, and autonomous activation of biochemical pathways regulated by growth factors. Some examples of RTKs involved in tumorigenesis are the receptors for epidermal growth factor receptor (EGFR) (also known as human EGF receptor-1 (HER1)), platelet-derived growth factor (PDGFR), insulin-like growth factor (IGFR), nerve growth factor (NGFR), and fibroblast growth factor (FGF). Binding of growth factors to these cell surface receptors induces receptor activation, which initiates and modifies signal transduction pathways and leads to cell proliferation and differentiation.

Generally, RTKs have an extracellular region, a transmembrane hydrophobic region, and an intracellular region bearing a kinase domain. The first step in the activation of an RTK is ligand-induced dimerization leading to exposure of phosphorylation sites, activation of the intracellular kinase domain and recruitment of down-stream signaling molecules. The most commonly observed mode of RTK dimerization involves the “crosslinking” of two receptors having exposed dimerization interfaces by binding of a bivalent ligand. For EGFR, structural data published in recent years have led to the proposal of quite a different mechanism. In the absence of ligand, a distinct configuration of the receptor monomer occludes the dimerization interface of the receptor by burying it in an intramolecular “tether.” Ligand binding induces a conformational change in EGFR that exposes this dimerization site, promoting dimerization and receptor activation.

EGFR is a 170 kD membrane-spanning glycoprotein with an extracellular ligand binding domain, a transmembrane region and a cytoplasmic protein tyrosine kinase domain. Examples of ligands that stimulate EGFR include epidermal growth factor (EGF), transforming growth factor-α (TGF-α), heparin-binding growth factor (HBGF), β-cellulin, and Cripto-1. Binding of specific ligands results in EGFR autophosphorylation, activation of the receptor's cytoplasmic tyrosine kinase domain and initiation of multiple signal transduction pathways that regulate tumor growth and survival.

Growth factors that activate EGFR are also thought to play a role in tumor angiogenesis. Angiogenesis, which refers to the formation of capillaries from pre-existing vessels in the embryo and adult organism, is known to be a key element in tumor growth, survival and metastasis. It has been reported that EGFR mediated stimulation of tumor cells leads to increased expression of the angiogenic factors vascular endothelial growth factor (VEGF), interleukin-8 (IL-8), and basic fibroblast growth factor (bFGF), which can lead to activation of tumor-associated vascular endothelial cells. Stimulation of tumor associated vascular endothelial cells may also occur through activation of their own EGF receptors, by tumor produced growth factors such as TGF-α and EGF.

It has been reported that many human tumors express or overexpress EGFR. Expression of EGFR is correlated with poor prognosis, decreased survival, and/or increased metastasis. EGFR, because of this involvement in tumorigenesis, has been specifically targeted for anticancer therapies. These therapies have predominantly included either a monoclonal antibody that blocks binding of ligand to the extracellular domain of the receptor or a synthetic tyrosine kinase inhibitor that acts directly on the intracellular region to prevent signal transduction.

Cetuximab MAb (ERBITUX®) is a recombinant, human/mouse chimeric, monoclonal antibody composed of the Fv regions of a murine anti-EGFR antibody with human IgG1 heavy and kappa light chain constant regions and has an approximate molecular weight of 152 kDa. Cetuximab binds specifically to the extracellular domain of the human EGFR, and is an EGFR antagonist, which blocks ligand binding to EGFR, prevents receptor activation, and inhibits growth of tumor cells that express EGFR. Cetuximab has been approved for use in combination with or without irinotecan in the treatment of patients with epidermal growth factor receptor-expressing, metastatic colorectal cancer who are refractory or can not tolerate irinotecan-based chemotherapy. Cetuximab has been shown to be effective for treatment of psoriasis.

The crystal structure of an EGF-EGFR extracellular domain complex, wherein the receptor domain exists in dimeric form, has been provided Ogiso, H. et al., 2002, Cell 110, 775-787. The structure of an EGF-EGFR extracellular domain complex obtained by crystallization at low, non-physiological pH, wherein the receptor exists in monomeric form has also been provided Ferguson, K. M. et al., 2003, Mol Cell 11, 507-517. The structure of a transforming growth factor alpha (TGF-α)-EGFR extracellular domain complex in dimeric form has also been determined (Garrett, T. P. et al., 2002, Cell 110, 763-773).

However, the crystal structure of EGFR with an antagonist, particularly cetuximab Fab, has not been previously determined. The invention disclosed herein provides for the first time crystals and atomic coordinates of a complex of an EGFR extracellular domain and cetuximab Fab. Accordingly, the present invention provides methods for identifying potential mimetics by screening against at least a subset of the coordinates obtained from such a crystal. Mimetics may be assayed for biological activities to obtain EGFR antagonists useful for treatment of EGFR dependent conditions or diseases. EGFR antagonists interact with the receptor to inhibit EGFR tyrosine kinase activity, without limitation, by blocking ligand binding, inhibiting receptor dimerization, ultimately inhibiting receptor substrate phosphorylation, gene activation, and cellular proliferation. Preferably, the antagonists have substantially similar or improved effectiveness as compared to cetuximab. The antagonists are used for treatment of conditions associated with EGFR expression. Such diseases include tumors that express, or overexpress EGFR and which may be stimulated by a ligand of EGFR. Also included are hyperproliferative diseases stimulated by a ligand of EGFR.

### SUMMARY OF THE INVENTION

In one aspect, the present invention provides a crystal of a receptor-antibody complex comprising a receptor-antibody complex of an epidermal growth factor receptor (EGFR) extracellular domain and cetuximab Fab, wherein the crystal has a resolution determined by X-ray crystallography of better than about 5.0 Angstroms. Preferably, the crystal has a resolution determined by X-ray crystallography of better than about 4.0 Angstroms, more preferably better than about 3.0 Angstroms. Preferably the crystal belongs to space group P2₁ and has unit cell dimensions a=77.8 Å, b=70.9 Å, c=147.1 Å, and β=02.5°. Preferably, the crystal has atomic coordinates provided in Table 2.

In another aspect, the present invention provides a method for preparing a crystal of a complex of an epidermal growth factor receptor (EGFR) extracellular domain and cetuximab Fab comprising preparing a solution containing the extracellular domain of EGFR and cetuximab Fab fragment, and growing the crystal. Preferably the pH of the solution is about 6.0 to about 8.0.

In another aspect, the present invention provides a method of identifying a mimetic of cetuximab comprising comparing a three-dimensional structure of the mimetic with a three-dimensional structure determined for the above crystal complex. Preferably, the three dimensional structure of the mimetic is compared with at least a subset of the coordinates provided in Table 2.

In one embodiment, identifying a mimetic is carried out by comparing the three-dimensional structure of the mimetic against the coordinates of at least one EGFR amino acid bound by cetuximab Fab. Such EGFR amino acid is selected from the group consisting of Gln 384, Gln 408, Ser 418, Ser 440, Lys 465, Ser 468, and Asn 469. In one embodiment, the locations of atoms of the mimetic that contact EGFR correspond to atoms of cetuximab that contact EGFR. In yet another embodiment, screening is carried out by comparing a three dimensional structure of a mimetic with the atomic coordinates of a region of EGFR selected from the group consisting of about amino acid residue 350 to about amino acid residue 354, about amino acid residue 380 to about amino acid residue 385, about amino acid residue 405 to about amino acid residue 420, about amino acid residue 435 to about amino acid residue 475 and combinations thereof.

The mimetic may be a small molecule, a peptide, or a polypeptide, preferably an antibody or a fragment thereof.

In another aspect of the invention, a mimetic that is an antibody or a fragment thereof is identified by introducing one or more substitutions in at least a single CDR region of cetuximab and/or at non-CDR amino acids of the antibody that interact with the CDR and affect its conformation. In one embodiment, at most a single substitution is made in each CDR. In another embodiment, substitution are made solely in CDR3 or at amino acids that affect the conformation of CDR3.

In another aspect, the present invention provides the above methods carried out with use of a computer.

The invention further provides a method for synthesizing the mimetic and assaying its binding or physiological activity to select EGFR antagonists useful for inhibiting EGFR function and treating EGFR-associated diseases or conditions. In an aspect of the invention, a mimetic is provided that inhibits tyrosine kinase activity of the receptor. In another aspect of the invention, the mimetic inhibits dimerization of EGFR expressed by a cell. Preferably, the mimetic blocks binding of EGF to EGFR. Mimetics of the invention bind to EGFR and inhibit EGFR functional activity, preferably to a similar or greater extent than cetuximab.

In another aspect, the present invention provides a computer-assisted method for identifying a mimetic of cetuximab comprising a processor, a data storage system, an input device, and an output device, comprising: inputting into the programmed computer through said input device data comprising the three-dimensional coordinates of at least a subset of the atoms of EGFR as set out in Table 2; providing a database of chemical and peptide structures stored in said computer data storage system; selecting from said database, using computer methods, structures having a portion that is structurally similar to said criteria data set; and outputting to said output device the selected chemical structures having a portion similar to said criteria data set.

In another aspect, the present invention provides a machine-readable medium having stored thereon a plurality of executable instructions to perform a method to identify a mimetic of cetuximab using a crystal of a receptor-antibody complex comprising a receptor-antibody complex of an epidermal growth factor receptor (EGFR) extracellular domain and cetuximab Fab, the method comprising: comparing a three-dimensional structure of a mimetic with a three dimensional structure an epidermal growth factor receptor (EGFR) extracellular domain and cetuximab Fab having an X-ray crystallography resolution of better than about 5.0 Angstroms.

Preferably the EGFR coordinates comprise at least a subset of the atomic coordinates of Table 2. In one embodiment, identifying a mimetic comprises comparing the three-dimensional structure of a mimetic with a three-dimensional structure of at least one EGFR amino acid bound by cetuximab Fab. In another embodiment identifying a mimetic comprises comparing a three dimensional structure of a mimetic with the atomic coordinates of a region of EGFR selected from the group consisting of about amino acid residue 350 to about amino acid residue 354, about amino acid residue 380 to about amino acid residue 385, about amino acid residue 405 to about amino acid residue 420, about amino acid residue 435 to about amino acid residue 475 and combinations thereof.

In another aspect, the present invention provides a machine-readable medium having stored thereon a plurality of executable instructions to perform a method for identifying a mimetic of cetuximab, the method comprising: introducing in silico substitutions in at least a single CDR region of cetuximab to obtain a pool of variants; and using a computer and at least a subset of the EGFR coordinates provided in Table 2 to select a variant with improved EGFR binding characteristics.

In another aspect, the present invention provides a cetuximab mimetic identified by any of the above methods.

In another aspect, the present invention provides a method of inhibiting EGFR comprising administering the identified mimetic.

In another aspect, the present invention provides a method of treating a disease or condition associated with EGFR expression comprising administering the identified mimetic. In one non-limiting embodiment, the present invention provides a method of inhibiting growth of a tumor cell that expresses EGFR comprising administering the above identified mimetics. In another embodiment, the present invention provides a method of treating a hyperproliferative diseases stimulated by a ligand of EGFR.

In another aspect, the present invention provides a method of treating psoriasis comprising administering the above identified mimetics.

### BRIEF DESCRIPTION OF THE FIGURES

FIG. 1 provides pictures of the crystals of the present invention. In this figure crystals on the left (1a) are representative of those used to collect the data and solve the structure. On the right (1b) are examples of crystals grown from the same condition except without CdCl₂, which are substantially identical to that obtained in the presence of CdCl₂.

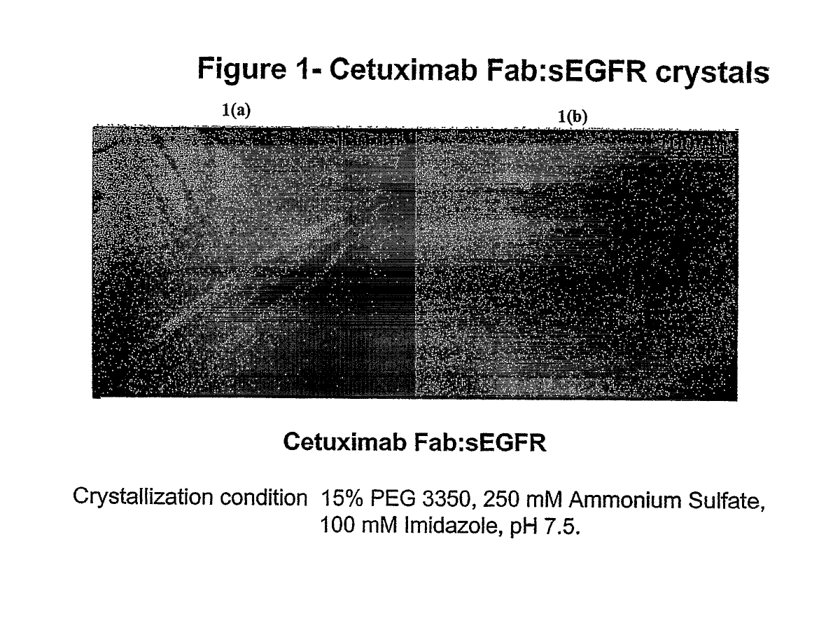

FIG. 2 provides various characteristics of the crystals of the present invention.

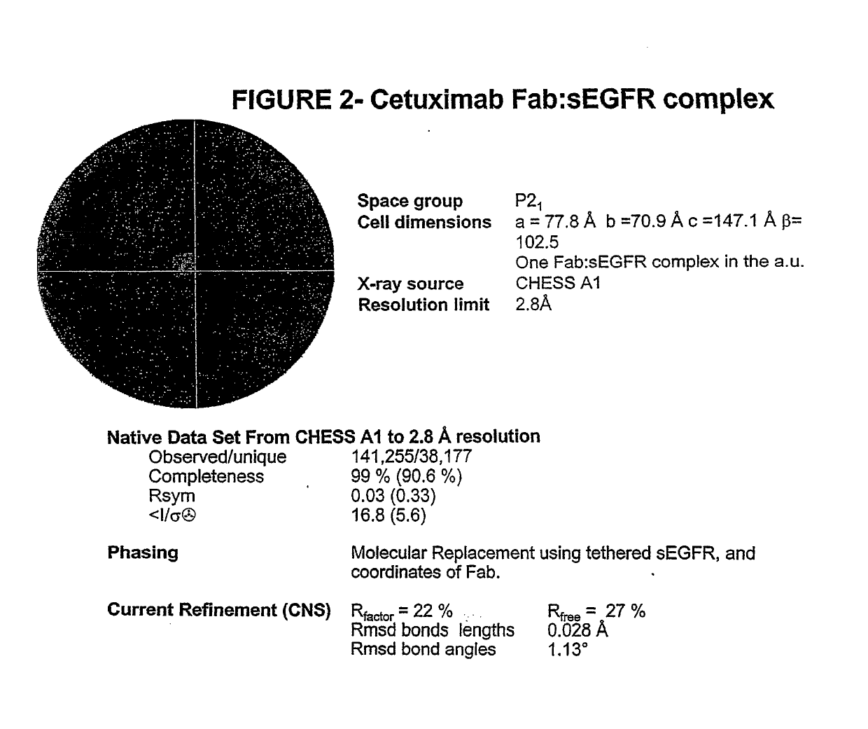

FIG. 3 provides crystal structures of EGFR and cetuximab Fab complex.

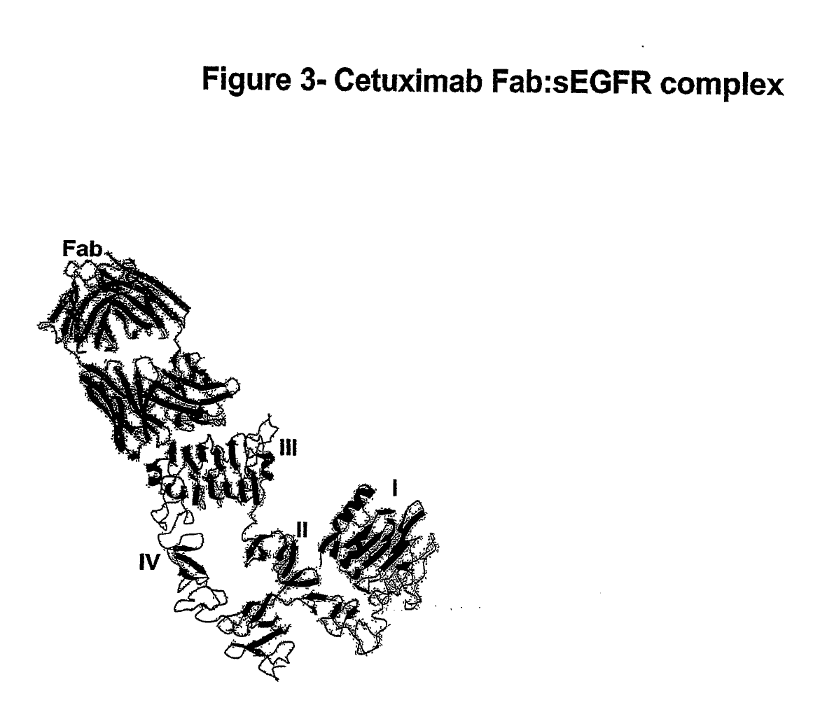

FIG. 4 provides graphs depicting affinity of cetuximab Fab, and affinity of EGFR ligands for EGFR, and also provides a competition assay. Additional details are provided in the example below having the heading “BIAcore binding studies.”

### DETAILED DESCRIPTION OF THE INVENTION

The present invention provides a co-crystal of EGFR extracellular domain and cetuximab Fab fragment with a resolution that is preferably greater than about 5 Å, more preferably greater than about 4 Å and most preferably greater than about 3 Å. The crystal preferably has a space group P2₁ and unit cell dimensions of a=77.8 Å, b=70.9 Å, c=147.1 Å; and β=102.5°.

To obtain the crystal for which structural coordinates are shown Table 2, the entire extracellular region (i.e., amino acids 1-618 of mature EGFR, including domains I, II, III and IV) is used, plus a C-terminal hexa-histidine tag (Ferguson, K. M. et al., 2000, Embo J 19, 4632-4643; Ferguson, K. M. et al., 2003, Mol Cell 11, 507-517). (See GenBank Accession No. 1NQLA). Cetuximab Fab contains the Fab fragment of Cetuximab, i.e., the heavy and light chain variable region sequences of murine antibody M225 (U.S. App. Ser. No. 2004/0006212, incorporated herein by reference) with human IgG1 C_{H}1 heavy and kappa light chain constant domains. (Cetuximab includes all three IgG1 heavy chain constant domains.) The CDR regions of the heavy chain of Cetuximab have the following sequences: a CDR1 region with a sequence of NY G V H, a CDR2 region with a sequence of V I W S G G N T D Y N T P F T S, and a CDR3 region with a sequence of A L T Y Y D Y E F A Y. The CDR regions of the light chain of Cetuximab have the following sequences: a CDR1 region with a sequence of R A S Q S I G T N I H, a CDR2 region with a sequence of Y A S E S I S, and a CDR3 region with a sequence of Q Q N N N W P T T.

The sequences of the proteins in the crystal, i.e., cetuximab Fab and the extracellular domain of EGFR, are also reported with the atomic coordinates of Table 2, except for amino acid positions at which the electron density map was insufficient to place all atoms of the actual amino acid side chain. At those positions, other amino acid side chains are designated.

Crystallization of the EGFR:cetuximab Fab complex may be carried out from a solution of cetuximab Fab and EGFR with various techniques, such as microbatch, hanging drop, sitting drop, sandwich drop, seeding and dialysis. The solution is prepared by combining EGFR extracellular domain with cetuximab Fab in a suitable buffer. A standard buffering agent such as Hepes, Tris, MES and acetate may be used. The buffer system may also be manipulated by addition of a salt such as sodium chloride, ammonium sulfate, sodium/potassium phosphate, ammonium acetate among others. Imidazole may also be used as a buffer. The concentration of the salt is preferably about 10 mM to about 500 mM, more preferably about 25 mM to about 100 mM, and most preferably about 50 mM. The pH of the buffer is preferably about 6 to about 8, more preferably about 7 to about 8. The concentration of the protein in the solution is preferably that of super-saturation to allow precipitation. The solution may optionally contain a protein stabilizing agent.

In one embodiment, the crystal is precipitated by contacting the solution with a reservoir that reduces the solubility of the proteins due to presence of precipitants, i.e., reagents that induce precipitation. Such contacting may be carried out through vapor diffusion. Examples of precipitants include ammonium sulfate, ethanol, 3-ethyl-2,4 pentanediol, and glycols, particularly polyethanol glycol (PEG). The PEG utilized preferably has a molecular weight of about 400 to about 20,000, more preferably about 3000 Da, with a concentration of about 10% to about 20%, more preferably about 15% (w/v). Some precipitants may act by making the buffer pH unfavorable for protein solubility.

The temperature during crystallization is preferably of about 0° C. to about 30° C., more preferably about 20° C. to about 30° C., and most preferably about 25° C. In addition to generation of structure, the crystallization technique of the invention may also be used to increase purity of proteins.

Precipitation may also be carried out in the presence of a heavy metal such as cadmium to further improve analysis of the crystal after precipitation.

In one embodiment illustrated in the example, about 0.5 μl (or microliter) protein at 11 mg/ml in 10 mM Hepes, 50 mM NaCl, pH 7.5 is contacted with 0.5 μl (or microliter) reservoir solution of about 15% PEG 3350, about 250 mM ammonium sulfate, about 10 mM cadmium chloride, about 100 mM imidazole and about pH 7.5. Essentially the same crystals are obtained without use of cadmium chloride. Crystals have also been grown at 15% PEG 3450, 100 mM CaCl₂, 50 mM Sodium acetate, pH 5.0 and 15% PEG 3450, 100 mM ammonium acetate, 50 mM sodium citrate, pH 5.0.

The atomic coordinates of the crystal of the present invention are disclosed in Table 2. The coordinates provide a three dimensional structure of the EGFR extracellular domain:cetuximab Fab complex of the crystal. The cetuximab Fab includes the portion of cetuximab which binds to the extracellular region of EGFR, and can be used to model the interaction of cetuximab and EGFR. Accordingly, the crystal and the deduced atomic coordinates allows for studying the binding interaction of cetuximab with EGFR and EGFR inhibition. The three dimensional structure further allows for identifying potential mimetics by screening potential mimetics against at least part of the structure (a subset of atoms provided in Table 2).

The three dimensional structure of EGFR:cetuximab Fab complex as defined by atomic coordinates is obtained from the X-ray diffraction pattern of the crystal and the electron density map derived therefrom. One method for determining the three dimensional structure is by molecular replacement which involves use of the structure of a closely related molecule or receptor ligand complex. An alternative method employs heavy atom derivatives.

One of skill in the art will also appreciate that the atomic coordinates provided are not precise, but are obtained from electron density measured for the crystal. Initial coordinates are determined by matching the protein backbone and side chains to the electron density map. The coordinates are refined by minimizing the overall energy of the protein (e.g., by adjusting bond lengths and angles), in view of the determined electron density. In some locations in the atomic structure, atoms of amino acid side chains may not be fully resolved due to, for example, solvent interactions and the like. Accordingly, the side chain that is modeled may differ from the actual side chain at that amino acid position. For the atomic coordinates set forth in Table 2, Arg 18 of the light chain and Gln 1 of the heavy chain of cetuximab Fab are modeled as Alanine. The present invention encompasses structures having root mean square deviations of backbone atoms of not more than about 1.5 Å, or more preferably not more than about 1.0 Å, or most preferably, not more than about 0.5 Å for residues of EGFR extracellular domain or cetuximab Fab that are used in identifying mimetics. The present invention encompasses variations within acceptable standards of error in the art for a crystal with the resolution disclosed herein.

It will also be appreciated that the origin of the atomic coordinates is arbitrarily defined. Accordingly, the same atomic structure can be represented by sets of coordinates that are numerically different, but that identify the same atomic positions. The present invention encompasses such alternative coordinate sets.

Identification of mimetics of cetuximab may be carried out with only a subset of the coordinates provided, such as those of amino acid residues of EGFR or cetuximab Fab that are associated in the complex.

Potential mimetics are examined against EGFR, particularly one or more of the above residues, through the use of computer modeling using a docking program. Such computer modeling allows for obtaining a positive initial indication of binding before synthesis and testing of the compound. If the testing shows sufficient interaction, then the compound may be synthesized and tested as a potential candidate. There is no limitation to the source of potential mimetics. For example, potential mimetics include structural databases of small molecules and other ligands represented in silico, as well as commercially available libraries of small molecules that can be similarly modeled. Potential mimetics further include peptides and macromolecules such as proteins, polypeptides, preferably antibodies or antibody fragments, synthetic polymer backbones having amino acid-like functional groups, and the like. Such potential mimetics may have defined structure, or be modeled on the basis of their similarity to other macromolecules of known structure. Iterative methods may be employed to vary one or more of the functional groups to improve the fit of the potential mimetic with EGFR. Those substances identified as mimetics, if not otherwise available to be tested for EGFR antagonist activity, may be synthesized.

In preferred embodiments, the locations of at least some atoms of cetuximab mimetics that contact EGFR correspond to the locations of atoms of cetuximab that contact EGFR. The correspondence is preferably within about 2.0 Å, more preferably within about 1.0 Å, and most preferably with about 0.5 Å. The atoms usually interact with EGFR in a manner similar to the corresponding atoms of cetuximab Fab (i.e., polar, basic, acidic, hydrophobic). The mimetics may contain various numbers of such corresponding atoms, and binding of the mimetic to EGFR may be completely or only partially dependent on such corresponding interactions. In certain embodiments, such atomic interactions with EGFR may be supplemented by interactions of other atoms of the mimetic that also interact with EGFR. The binding ability of the mimetics can be evaluated by various computer programs as disclosed herein.

Docking may be accomplished by using software such as Quanta and Sybyl (manual model building software), followed by energy minimization and molecular dynamics with standard molecular mechanics force fields, such as CHARMM and AMBER. Specialized programs for docking include GRAM, GRID, Flexx, Glide, GOLD, MCSS, DOCK or AUTODOCK (See e.g. U.S. Pat. Nos. 5,856,116 and 6,087,478; Jorgensen W. L., 2004, Science 303, 1813-1818). Such procedure includes computer fitting of potential antagonists to EGFR to determine how the three dimensional structure of EGFR and the chemical properties of each amino acid interfere with EGFR activation, and to estimate attraction, repulsion and steric hindrance of the binding. Generally, tighter fits are preferred in that they are more likely to be effective when administered in vivo, and would be more selective for EGFR, minimizing binding to other receptors. Many of these programs also consider adsorption, distribution, metabolic and excretion characteristics of the molecules.

The docking program may be connected to a structure generator (such as SYNOPSIS) to perform de novo screening. An alternative to de novo screening, is creation of structures based on the binding site such as with programs including LUDI, SPROUT and BOMB, which allow a user to put a substituent in a binding site and then build up the substituent (Jorgensen W. L., 2004).

One of skill in the art would appreciate that the above screening methods may also be carried out manually, by building an actual three dimensional model based on the coordinates, and then determining desirable antagonists based on that model visually.

Of particular interest for designing mimetics are those amino acids that overlap with the binding site of EGR or TGF-α to EGFR. Such binding may interfere with the ligand-induced dimerization of the receptor or inhibit binding of the ligand to EGFR altogether.

Domains I and III of EGFR are responsible for binding of EGF to the receptor, and are of interest in designing antagonists. Of the amino acids of EGFR, some are involved in direct hydrogen bonding with cetuximab Fab. These amino acids include Ser 468, Asn 469, Arg 353, Gln 384, Gln 408, Ser 418, Ser 440 and/or Lys 465. Ser468 and Asn 469 are involved in main-chain hydrogen bonds, i.e., the nature of the side chain is not directly relevant. Antagonists may be designed to bind to a few, most or none of these amino acids. Other amino acids of EGFR are in contact to some lesser degree with cetuximab Fab. These amino acids include: Pro 349, Arg 353, Leu 382, His 409, Phe 412, Val 417, Ser 418, Ile 438, Gly 441, Lys 443, Ile 466, Ile 467, Gln 471 and Asn 473. Of the nine amino acids between 465 and 473, eight of them are in some contact with cetuximab Fab. This region of EGFR is also ideal for screening of antagonists, particularly since residues 467 and 468 are in contact with both the heavy and light chains of cetuximab.

Cetuximab Fab does not bind to amino acids at positions 325, 346, 348, 350, 354-357 and 411, despite these amino acids being involved in EGF/TGF-α binding. Screening may be carried out against these positions, or only for the positions bound by cetuximab Fab, or both. If screening is carried out based on the binding of cetuximab Fab to EGFR, such screening may be carried out in regions of amino acids of about 350 to about 354, amino acids of about 380 to about 385, amino acids of about 405 to about 420, amino acids of about 435 to about 475 and combinations thereof. One of skill in the art would appreciate that screening may simply be carried out against domains I and III of EGFR based on the crystal structure provided, and general area of the binding pocket, without focus on any particular amino acids bound by cetuximab Fab and/or ligands.

The mimetics, both peptides and small organic molecules, preferably antibody and antibody fragments, bind to EGFR and mimic effects of cetuximab both in vivo and in vitro. In addition to peptides and small organic molecules, the mimetic may be a sugar. The mimetic may also be a combination of peptides/small molecules/sugars, such as a peptide having a synthetic backbone. The mimetic may be designed based on criteria such as affinity for EGFR, desirable efficacy and/or desirable selectivity. These mimetics have at least a single physiological or binding activity of cetuximab, which activity can be tested by assays provided further below.

As used herein, “mimetics” include cetuximab mimetics with modifications that retain specificity for EGFR. Such modifications include, but are not limited to, conjugation to an effector molecule such as a chemotherapeutic agent (e.g., cisplatin, taxol, doxorubicin) or cytotoxin (e.g., a protein, or a non-protein organic chemotherapeutic agent). The mimetics can be modified by conjugation to detectable reporter moieties. Also included are mimetics with alterations that affect non-binding characteristics such as half-life (e.g., pegylation).

Proteins and non-protein agents may be conjugated to the mimetics by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990) for the conjugation of doxorubicin and those described by Arnon et al., Adv. Exp. Med. Biol. 303, 79-90 (1991) and by Kiseleva et al., Mol. Biol. (USSR)25, 508-514 (1991) for the conjugation of platinum compounds.

In one embodiment, a library of small organic molecules is used to screen for mimetics in silico. In another embodiment, cetuximab is used as a starting candidate, and varied to generate a cetuximab variant with desirable properties. Such variant of cetuximab may be a scFv, a Fab, diabody, or IgG. For example, conservative amino acid substitutions may be made at one or more of residues of cetuximab Fab which bind EGFR: light chain (LC) residues Asn 91, Trp 94; heavy chain (HC) residues Gly 54, Tyr 102, Trp 52, Asp 103.

A conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical substitutions that may be performed for such conservative amino acid substitution may be among the groups of amino acids as follows:

glycine (G), alanine (A), valine (V), leucine (L) and isoleucine (I);
aspartic acid (D) and glutamic acid (E);
alanine (A), serine (S) and threonine (T);
histidine (H), lysine (K) and arginine (R):
asparagine (N) and glutamine (Q);
phenylalanine (F), tyrosine (Y) and tryptophan (W).

If the binding is not as tight in regard to one or more of the residues, less conservative substitutions may be made at those residues to optimize the binding. For example, an amino acid with a hydrophilic group may be substituted for one with a hydrophobic group.

In one embodiment, a mixture of all or some amino acids is introduced to synthesize variants of cetuximab randomly at specified positions in silico: Tyr 102 (HC), Trp 52 (HC), and Asp 103 (HC) of cetuximab. Only these amino acid residues are involved in side chain hydrogen bonds, and thus are candidates for specific mutations aimed at modifying direct interactions. Such variation, where all 20 amino acids are used, would result in about 20³ variants which can then be screened. If only conservative substitutions are made, the variation would be much less, about 3³. Conservative and non-conservative substitutions at other positions in the CDRs of cetuximab that do not bind to EGFR directly should also be considered. For example, direct interactions between contact residues (e.g., main chain—main chain, main chain—side chain, side chain—side chain contacts) can be modified by introducing changes at amino acid positions that affect the position of cetuximab side chain and main chain atoms involved in direct interactions with EGFR. In one embodiment, at most a single substitution is made in each CDR. In another embodiment a single substitution is made in the heavy chain CDR3 region of cetuximab.

After such screening and selection, the selected mimetic may be synthesized, and various assays carried out to measure the biological or physiological activity of the mimetic to select an EGFR antagonist. A preferred EGFR antagonist has one or more of the following properties: inhibits EGFR tyrosine kinase activity; blocks ligand binding to EGFR; inhibits EGFR dimerization (homodimerization with EGFR or heterodimerization with another EGFR family receptor subunit); inhibits EGFR substrate phosphorylation; inhibits EGFR mediated gene activation; inhibits growth or proliferation of a cell the expresses EGFR. Preferably, the antagonist has substantially similar or improved effectiveness as an EGFR antagonist as compared to Cetuximab.

Tyrosine kinase inhibition can be determined using well-known methods; for example, by measuring the autophosphorylation level of recombinant kinase receptor, and/or phosphorylation of natural or synthetic substrates. Thus, phosphorylation assays are useful in determining EGFR antagonists of the present invention. Phosphorylation can be detected, for example, using an antibody specific for phosphotyrosine in an ELISA assay or on a western blot. Some assays for tyrosine kinase activity are described in Panek et al., J. Pharmacol. Exp. Thera. (1997) 283: 1433-44 and Batley et al., Life Sci. (1998) 62: 143-50.

In addition, methods for detection of protein expression can be utilized to determine EGFR antagonists, wherein the proteins being measured are regulated by EGFR tyrosine kinase activity. These methods include immunohistochemistry (IHC) for detection of protein expression, fluorescence in situ hybridization (FISH) for detection of gene amplification, competitive radioligand binding assays, solid matrix blotting techniques, such as Northern and Southern blots, reverse transcriptase polymerase chain reaction (RT-PCR) and ELISA. See, e.g., Grandis et al., Cancer, (1996) 78:1284-92; Shimizu et al., Japan J. Cancer Res., (1994) 85:567-71; Sauter et al., Am. J. Path., (1996) 148:1047-53; Collins, Glia, (1995) 15:289-96; Radinsky et al., Clin. Cancer Res., (1995) 1:19-31; Petrides et al., Cancer Res., (1990) 50:3934-39; Hoffmann et al., Anticancer Res., (1997) 17:4419-26; Wikstrand et al., Cancer Res., (1995) 55:3140-48.

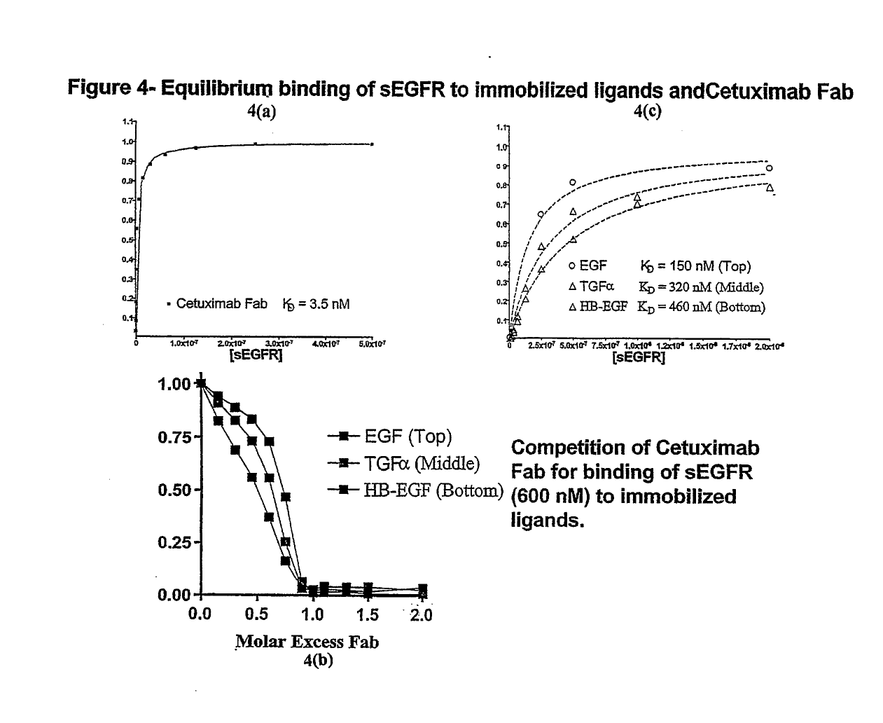

The ability of a mimetic to block ligand binding can be measured, for example, by an in vitro competitive assay such as is illustrated in FIG. 4. In this assay, a ligand of EGFR such as EGF is immobilized, and a binding assay is carried to determine the effectiveness of the mimetic to competitively inhibit binding of EGFR to the immobilized ligand.

In vivo assays can also be utilized to determine EGFR antagonists. For example, receptor tyrosine kinase inhibition can be observed by mitogenic assays using cell lines stimulated with receptor ligand in the presence and absence of inhibitor. For example, A431cells (American Type Culture Collection (ATCC), Rockville, Md.) stimulated with EGF can be used to assay EGFR inhibition. Another method involves testing for inhibition of growth of EGFR-expressing tumor cells, using for example, human tumor cells injected into a mouse. See U.S. Pat. No. 6,365,157 (Rockwell et al.).

The present invention provides for coordinates of the co-crystal of the present invention on a computer readable format such as a magnetic disk, CD-ROM or a hard drive.

In another aspect, the present invention provides methods of treating EGFR-dependent diseases and conditions in mammals by administering a therapeutically effective amount of a mimetic of cetuximab. One skilled in the art would easily be able to diagnose such conditions and disorders using known, conventional tests. Treatment means any treatment of a disease in an animal and includes: (1) preventing the disease from occurring in a mammal which may be predisposed to the disease but does not yet experience or display symptoms of the disease; e.g., prevention of the outbreak of the clinical symptoms; (2) inhibiting the disease, e.g., arresting its development; or (3) relieving the disease, e.g., causing regression of the symptoms of the disease. Therapeutically effective amount for the treatment of a disease means that amount which, when administered to a mammal in need thereof, is sufficient to effect treatment, as defined above, for that disease. A cetuximab mimetic of the invention may be administered with an antineoplastic agent such as, for example, a chemotherapeutic.

Cetuximab mimetics of the present invention are useful for treating tumors that express EGFR. EGFR expressing tumors are characteristically sensitive to EGF present in their environment, and can further be stimulated by tumor produced EGF or TGF-α. While not intending to be bound to any particular mechanism, the diseases and conditions that may be treated or prevented by the present methods include, for example, those in which tumor growth is stimulated through an EGFR paracrine and/or autocrine loop. The method is therefore effective for treating a solid tumor that is not vascularized, or is not yet substantially vascularized.

In another aspect of the invention, cetuximab mimetics are used to inhibit tumor-associated angiogenesis. EGFR stimulation of vascular endothelium is associated with vascularization of tumors. Typically, vascular endothelium is stimulated in a paracrine fashion by EGF and/or TGF-α from other sources (e.g., tumor cells). Accordingly, the cetuximab mimetics are effective for treating subjects with vascularized tumors or neoplasms.

Tumors that may be treated include primary tumors and metastatic tumors, as well as refractory tumors. Refractory tumors include tumors that fail to respond or are resistant to treatment with chemotherapeutic agents alone, antibodies alone, radiation alone or combinations thereof. Refractory tumors also encompass tumors that appear to be inhibited by treatment with such agents, but recur up to five years, sometimes up to ten years or longer after treatment is discontinued. The tumors may express EGFR at normal levels or they may overexpress EGFR at levels, for example, that are at least 10, 100, or 1000 times normal levels.

Examples of tumor that express EGFR and are stimulated by a ligand of EGFR include carcinomas, gliomas, sarcomas, adenocarcinomas, adenosarcomas, and adenomas. Such tumors can occur in virtually all parts of the body, including, for example, breast, heart, lung, small intestine, colon, spleen, kidney, bladder, head and neck, ovary, prostate, brain, pancreas, skin, bone, bone marrow, blood, thymus, uterus, testicles, cervix or liver. Some tumors observed to overexpress EGFR that may be treated according to the present invention include, but are not limited to, colorectal and head and neck tumors, especially squamous cell carcinoma of the head and neck, brain tumors such as glioblastomas, and tumors of the lung, breast, pancreas, esophagus, bladder, kidney, ovary, cervix, and prostate. Non-limiting examples of tumors observed to have constitutively active (i.e., unregulated) receptor tyrosine kinase activity include gliomas, non-small-cell lung carcinomas, ovarian carcinomas and prostate carcinomas. Other examples of tumors include Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma, preferably glioblastoma multiforme, and leiomyosarcoma.

The present invention also provides a method of treating a non-cancer hyperproliferative disease in a mammal comprising administering to the mammal an effective amount of the antibody of the present invention. As disclosed herein, “hyperproliferative disease” is defined as a condition caused by excessive growth of non-cancer cells that express a member of the EGFR family of receptors. The excess cells generated by a hyperproliferative disease express EGFR at normal levels or they may overexpress EGFR.

The types of hyperproliferative diseases that can be treated in accordance with the invention are any hyperproliferative diseases that are stimulated by a ligand of EGFR or mutants of such ligands. Examples of hyperproliferative disease include psoriasis, actinic keratoses, and seborrheic keratoses, warts, keloid scars, and eczema. Also included are hyperproliferative diseases caused by virus infections, such as papilloma virus infection. For example, psoriasis comes in many different variations and degrees of severity. Different types of psoriasis display characteristics such as pus-like blisters (pustular psoriasis), severe sloughing of the skin (erythrodermic psoriasis), drop-like dots (guttae psoriasis) and smooth inflamed lesions (inverse psoriasis). The treatment of all types of psoriasis (e.g., psoriasis vulgaris, psoriasis pustulosa, psoriasis erythrodermica, psoriasis arthropathica, parapsoriasis, palmoplantar pustulosis) is contemplated by the invention.

Administering the cetuximab mimetic includes delivering the mimetic to a mammal by any method that may achieve the result sought. The term mammal as used herein is intended to include, but is not limited to, humans, laboratory animals, domestic pets and farm animals. The mimetic may be administered, for example, orally, parenterally (intravenously or intramuscularly), topically, transdermally or by inhalation. Topical administration may be preferred for certain hyperproliferative disorders.

In an embodiment of the invention, cetuximab mimetic can be administered in combination with one or more other anti-neoplastic agents, such as chemotherapeutic agents. Radiation can also be employed. For examples of combination therapies, see, e.g., U.S. Pat. No. 6,217,866 (Schlessinger et al.) (Anti-EGFR antibodies in combination with anti-neoplastic agents); WO 99/60023 (Waksal et al.) (Anti-EGFR antibodies in combination with radiation). Any suitable anti-neoplastic agent can be used, such as a chemotherapeutic agent, radiation or combinations thereof. The anti-neoplastic agent can be an alkylating agent or an anti-metabolite. Examples of alkylating agents include, but are not limited to, cisplatin, cyclophosphamide, melphalan, and dacarbazine. Examples of anti-metabolites include, but not limited to, doxorubicin, daunorubicin, paclitaxel, irinotecan (CPT-11), and topotecan. When the agent is radiation, the source of the radiation can be either external (external beam radiation therapy—EBRT) or internal (brachytherapy—BT) to the patient being treated. The dosage administered depends on numerous factors, including, for example, the type of agent, the type and severity tumor being treated and the route of administration of the agent. It should be emphasized, however, that the present invention is not limited to any particular dose.

For treatment of hyperproliferative disease, the cetuximab mimetic can be combined with any conventional treatment agent. For example, when the hyperproliferative disease is psoriasis, there are a variety of conventional systemic and topical agents available. Systemic agents for psoriasis include methotrexate, and oral retinoids, such as acitretin, etretinate, and isotretinoin. Other systemic treatments of psoriasis include hydroxyurea, NSAIDS, sulfasalazine, and 6-thioguanine. Antibiotics and antimicrobials can be used to treat or prevent infection that can cause psoriasis to flare and worsen. Topical agents for psoriasis include anthralin, calcipotriene, coal tar, corticosteroids, retinoids, keratolytics, and tazarotene. Topical steroids are one of the most common therapies prescribed for mild to moderate psoriasis. Topical steroids are applied to the surface of the skin, but some are injected into the psoriasis lesions.

Hyperproliferative disease treatments further include administration of the cetuximab mimetic in combination with phototherapy. Phototherapy includes administration of any wavelength of light that reduces symptoms of the hyperproliferative disease, as well as photoactivation of a chemotherapeutic agent (photochemotherapy). For further discussion of treatment of hyperproliferative disorders, see WO 02/11677 (Teufel et al.) (Treatment of hyperproliferative diseases with epidermal growth factor receptor antagonists).

In certain embodiments of the invention, cetuximab mimetics of the invention can be administered with EGFR antagonists and/or antagonists of other receptors involved in tumor growth or angiogenesis. The receptor antagonists may bind to the receptor or the ligand to block receptor-ligand binding, or the receptor antagonists may otherwise neutralize the receptor tyrosine kinase. Ligands of EGFR include, for example, EGF, TGF-α amphiregulin, heparin-binding EGF (HB-EGF) and betacellulin. EGF and TGF-α are thought to be the main endogenous ligands that result in EGFR-mediated stimulation, although TGF-α has been shown to be more potent in promoting angiogenesis. Accordingly, EGFR antagonists include antibodies that bind to such ligands and thereby block binding to and activation of EGFR.

The cetuximab mimetic can be used in combination with a VEGFR antagonist. In one embodiment of the invention, a cetuximab mimetic is used in combination with a receptor antagonist that binds specifically to VEGFR-2/KDR receptor (PCT/US92/01300, filed Feb. 20, 1992; Terman et al., Oncogene 6: 1677-1683 (1991)). In another embodiment of the invention, a cetuximab mimetic is used in combination with a receptor antagonist that binds specifically to VEGFR-1/Flt-1 receptor (Shibuya M. et al., Oncogene 5, 519-524 (1990)). In another embodiment, a cetuximab mimetic is used in combination with a receptor antagonist that binds to a VEGFR ligand. For example, Avastin® (bevacizumab) is an antibody that binds VEGF. Particularly preferred are antigen-binding proteins that bind to the extracellular domain of VEGFR-1 or VEGFR-2 and block binding by ligand (VEGF or P1GF), and/or neutralize VEGF-induced or P1GF-induced activation. For example, Mab IMC-1121 binds to soluble and cell surface-expressed KDR. Mab IMC-1121 comprises the V_{H} and V_{L} domains obtained from a human Fab phage display library. (See WO 03/075840) In another example, ScFv 6.12 binds to soluble and cell surface-expressed Flt-1. ScFv 6.12 comprises the V_{H} and V_{L} domains of mouse monoclonal antibody MAb 6.12. A hybridoma cell line producing MAb 6.12 has been deposited as ATCC number PTA-3344.

In another embodiment, a cetuximab mimetic is administered with an antagonist of insulin-like growth factor receptor (IGFR). In certain tumor cells, inhibition of EGFR function can be compensated by upregulation of other growth factor receptor signaling pathways, and particularly by IGFR stimulation. Further, inhibition of IGFR signaling results in increased sensitivity of tumor cells to certain therapeutic agents. Stimulation of either EGFR or IGFR results in phosphorylation of common downstream signal transduction molecules, including Akt and p44/42, although to different extents. Accordingly, in an embodiment of the invention, an IGFR antagonist (e.g., an antibody that binds to IGF or IGFR and neutralizes the receptor) is coadministered with a cetuximab mimetic of the invention, thereby blocking a second input into the common downstream signaling pathway (e.g., inhibiting activation of Akt and/or p44/42). An example of a human antibody specific for IGFR is IMC-A12 (See WO 2005/016970).

Other examples of growth factor receptors involved in tumorigenesis against which antagonists may be directed are the receptors for platelet-derived growth factor (PDGFR), hepatocyte growth factor (HGFR), nerve growth factor (NGFR), fibroblast growth factor (FGFR), and macrophage stimulating protein (RON).

The cetuximab mimetics can also be administered with intracellular RTK antagonists that inhibit activity of RTKs or their associated downstream signaling elements that are involved in tumor growth or tumor-associated angiogenesis. The intracellular RTK antagonists are preferably small molecules. Some examples of small molecules include organic compounds, organometallic compounds, salts of organic compounds and organometallic compounds, and inorganic compounds. Atoms in a small molecule are linked together via covalent and ionic bonds; the former is typical for small organic compounds such as small molecule tyrosine kinase inhibitors and the latter is typical of small inorganic compounds. The arrangement of atoms in a small organic molecule may represent a chain, e.g. a carbon-carbon chain or carbon-heteroatom chain or may represent a ring containing carbon atoms, e.g. benzene or a policyclic system, or a combination of carbon and heteroatoms, i.e., heterocycles such as a pyrimidine or quinazoline. Although small molecules can have any molecular weight, they generally include molecules that would otherwise be considered biological molecules, except their molecular weight is not greater than 650 D. Small molecules include both compounds found in nature, such as hormones, neurotransmitters, nucleotides, amino acids, sugars, lipids, and their derivatives as well as compounds made synthetically, either by traditional organic synthesis, bio-mediated synthesis, or a combination thereof. See e.g. Ganesan, Drug Doscov. Today 7(1): 47-55 (January 2002); Lou, Drug Discov. Today, 6(24): 1288-1294 (December 2001).

More preferably, the small molecule to be used as an intracellular RTK antagonist according to the present invention is an intracellular EGFR antagonist that competes with ATP for binding to EGFR's intracellular binding region having a kinase domain or to proteins involved in the signal transduction pathways of EGFR activation. Examples of such signal transduction pathways include the ras-mitogen activated protein kinase (MAPK) pathway, the phosphatidylinositol-3 kinase (P13K)-Akt pathway, the stress-activated protein kinase (SAPK) pathway, and the signal transducers and activators of transcription (STAT) pathways. Non-limiting examples of proteins involved in such pathways (and to which a small molecule EGFR antagonist according to the present invention can bind) include GRB-2, SOS, Ras, Raf, MEK, MAPK, and matrix metalloproteinases (MMPs).

One example of a small molecule EGFR antagonist is IRESSA™ (ZD1939), which is a quinozaline derivative that functions as an ATP-mimetic to inhibit EGFR. See U.S. Pat. No. 5,616,582 (Zeneca Limited); WO 96/33980 (Zeneca Limited) at p. 4; see also, Rowinsky et al., Abstract 5 presented at the 37th Annual Meeting of ASCO, San Francisco, Calif., 12-15 May 2001; Anido et al., Abstract 1712 presented at the 37th Annual Meeting of ASCO, San Francisco, Calif., 12-15 May 2001. Another example of a small molecule EGFR antagonist is TARCEVA™ (OSI-774), which is a 4-(substitutedphenylamino)quinozaline derivative [6,7-Bis(2-methoxy-ethoxy)-quinazolin-4-yl]-(3-ethynyl-phenylamine hydrochloride] EGFR inhibitor. See WO 96/30347 (Pfizer Inc.) at, for example, page 2, line 12 through page 4, line 34 and page 19, lines 14-17. See also Moyer et al., Cancer Res., 57: 4838-48 (1997); Pollack et al., J. Pharmacol., 291: 739-48 (1999). TARCEVA™ may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., 12-15 May 2001.

Other small molecules are also reported to inhibit EGFR, many of which are thought to being to the tyrosine kinase domain of an EGFR. Some examples of such small molecule EGFR antagonists are described in WO 91/116051, WO 96/30347, WO 96/33980, WO 97/27199 (Zeneca Limited). WO 97/30034 (Zeneca Limited), WO 97/42187 (Zeneca Limited), WO 97/49688 (Pfizer Inc.), WO 98/33798 (Warner Lambert Company), WO 00/18761 (American Cyanamid Company), and WO 00/31048 (Warner Lambert Company). Examples of specific small molecule EGFR antagonists include C1-1033 (Pfizer), which is a quinozaline (N-[4-(3-chloro-4-fluoro-phenylamino)-7-(3-morpholin-4-yl-propoxy)-quinazolin-6-yl]-acrylamide) inhibitor of tyrosine kinases, particularly EGFR and is described in WO 00/31048 at page 8, lines 22-6; PKI166 (Novartis), which is a pyrrolopyrimidine inhibitor of EGFR and is described in WO 97/27199 at pages 10-12; GW2016 (GlaxoSmithKline), which is an inhibitor of EGFR and HER2; EKB569 (Wyeth), which is reported to inhibit the growth of tumor cells that overexpress EGFR or HER2 in vitro and in vivo; AG-1478 (Tryphostin), which is a quinazoline small molecule that inhibits signaling from both EGFR and erbB-2; AG-1478 (Sugen), which is bisubstrate inhibitor that also inhibits protein kinase CK2; PD 153035 (Parke-Davis) which is reported to inhibit EGFR kinase activity and tumor growth, induce apoptosis in cells in culture, and enhance the cytotoxicity of cytotoxic chemotherapeutic agents; SPM-924 (Schwarz Pharma), which is a tyrosine kinase inhibitor targeted for treatment of prostrate cancer; CP-546,989 (OSI Pharmaceuticals), which is reportedly an inhibitor of angiogenesis for treatment of solid tumors; ADL-681, which is a EGFR kinase inhibitor targeted for treatment of cancer; PD 158780, which is a pyridopyrimidine that is reported to inhibit the tumor growth rate of A4431 xenografts in mice; CP-358,774, which is a quinzoline that is reported to inhibit autophosphorylation in HN5 xenografts in mice; ZD1839, which is a quinzoline that is reported to have antitumor activity in mouse xenograft models including vulvar, NSCLC, prostrate, ovarian, and colorectal cancers; CGP 59326A, which is a pyrrolopyrimidine that is reported to inhibit growth of EGFR-positive xenografts in mice; PD 165557 (Pfizer); CGP54211 and CGP53353 (Novartis), which are dianilnophthalimides. Naturally derived EGFR tyrosine kinase inhibitors include genistein, herbimycin A, quercetin, and erbstatin.

Further small molecules reported to inhibit EGFR and that are therefore within the scope of the present invention are tricyclic compounds such as the compounds described in U.S. Pat. No. 5,679,683; quinazoline derivatives such as the derivatives described in U.S. Pat. No. 5,616,582; and indole compounds such as the compounds described in U.S. Pat. No. 5,196,446.

In another embodiment, the EGFR antagonist can be administered in combination with one or more suitable adjuvants, such as, for example, cytokines (IL-10 and IL-13, for example) or other immune stimulators, such as, but not limited to, chemokine, tumor-associated antigens, and peptides. See, e.g., Larrivée et al., supra. It should be appreciated, however, that administration of only a cetuximab mimetic is sufficient to prevent, inhibit, or reduce the progression of the tumor in a therapeutically effective manner.

For combination therapies, the cetuximab mimetic and anti-neoplastic agent or receptor antagonist may be administered concomitantly or sequentially.

This invention also provides a pharmaceutical composition/formulation containing a cetuximab mimetic and a pharmaceutically acceptable carrier. Carrier as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate and other organic acids; antioxidants including ascorbic acid; low molecular weight (less than about 10 residues) polypeptide; proteins, such as serum albumin, gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

The active ingredients may also be entrapped in microcapsules prepared, for example, by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes. Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The present invention also includes kits for inhibiting tumor growth and/or tumor-associated angiogenesis comprising a therapeutically effective amount of a cetuximab mimetic. The kits can further contain any suitable antagonist of, for example, another growth factor receptor involved in tumorigenesis or angiogenesis (e.g., VEGFR-1/Flt-1, VEGFR-2, PDGFR, IGFR, NGFR, FGFR, etc, as described above). Alternatively, or in addition, the kits of the present invention can further comprise an anti-neoplastic agent. Examples of suitable anti-neoplastic agents in the context of the present invention have been described herein. The kits of the present invention can further comprise an adjuvant; examples have also been described above.

Moreover, included within the scope of the present invention is use of the present antibodies in vivo and in vitro for investigative or diagnostic methods, which are well known in the art. The diagnostic methods include kits, which contain mimetics of the present invention.

Accordingly, the mimetics can be used in vivo and in vitro for investigative, diagnostic, prophylactic, or treatment methods, which are well known in the art. Of course, it is to be understood and expected that variations in the principles of invention herein disclosed can be made by one skilled in the art and it is intended that such modifications are to be included within the scope of the present invention.

All references mentioned herein are incorporated by reference.

### Examples

The following examples are offered for illustrative purposes only, and are not intended to limit the scope of the present invention in any way.

Protein expression and purification. sEGFR was produced and purified from baculovirus-infected Sf9 cells as described by Ferguson, K. M. et al., 2000, Embo J 19, 4632-4643, and was used without modification of its glycosylation state. This sEGFR was further purified by size exclusion chromatography (SEC) using a SEC250 column (BioRad) pre-equilibrated with 25 mM HEPES, 100 mM NaCl, pH 7.5 and concentrated to 6.2 mg/ml. Cetuximab Fab fragment was prepared by treatment of the IgG protein with papain. The IgG protein (20 mg/ml) was incubated with papain (1:1000 w:w) at 37° C. for one hour and the digestion was terminated by addition of iodoacetemide (75 mM final concentration). The reaction mixture was loaded onto a Protein-A column and the flow-through fraction containing the Fab fragments was collected and concentrated. The cetuximab Fab was fractionated by SEC and mixed with sEGFR to give a two fold molar excess of Fab over sEGFR. Excess Fab was separated from the sEGFR:Fab complex using the same SEC column. The peak fractions containing the sEGFR:Fab complex (as confirmed by SDS-PAGE), were concentrated to 11 mg/ml.

Crystallization and data collection. The sEGFR:Fab complex was buffer-exchanged into 25 mM HEPES, pH 7.5, containing 50 mM NaCl, and crystallized by the hanging drop method from a drop containing equal parts of a 78 μM sEGFR:Fab complex solution and reservoir solution of 15% PEG3350, 250 mM (NH₄)₂SO₄, 100 mM imidazole, 10 mM CdCl₂, pH 7.5. Streak seeding was used to produce large (0.08×0.08×0.6 mm) single crystals. Crystals were cryo-stabilized with a brief exposure to 15% PEG3350, 15% Ethylene Glycol, 250 mM (NH₄)₂SO₄, 100 mM imidazole, 10 mM CdCl₂, pH 7.5, and were flash frozen in liquid nitrogen. Data were collected at CHESS beamline Al, using an ADSC Quantum-210 CCD detector, and were processed using HKL2000 (See, Otwinowski, Z., and Minor, W. (1997). Processing of X-ray Diffraction Data Collected in Oscillation Mode. In Macromolecular Crystallography, Volume 276, C. W. Carter Jr. and R. M. Sweet, eds. (New York: Academic Press), pp. 307-326.

Structure determination and refinement. Search models for molecular replacement were derived from the coordinates of tethered sEGFR (pdb id. 1NQL; Ferguson, K. M. et al., 2003) and those of the structure of the Fab fragment alone (P. Jeffrey and P. Kussie unpublished data). An initial solution was found for a domain I/II fragment (amino acids 5-240) combined with a domain III fragment of sEGFR using the dyad option of MOLREP (The CCP4 Suite: Programs for Protein Crystallography. Acta Cryst. D50, 760-763 (1990)) to search for the best relative orientation of these two fragments. With the solution for these fragments fixed it was possible to find a solution for the Fab fragment. Rigid body refinement with CNS was used to optimize the orientation of the individual sub-domains of the Fab. Following several rounds of model building using ‘O’ (Jones, T. A. et al., 1991, Acta Crystallogr A 47 (Pt 2), 110-119) and refinement using CNS (Brunger, A. T. et al., 1998, Acta Crystallogr D Biol Crystallogr 54 (Pt 5), 905-921), interpretable density for the remaining portions of sEGFR(C-terminal part of domain II and domain IV) could be seen in composite simulated-annealing omit-maps (calculated with CNS). The final stages of refinement employed TLS refinement (Winn, M. D. et al., 2001, Acta Crystallogr D Biol Crystallogr 57, 122-133) with anisotropic motion tensors refined for each of the four domains of sEGFR and each of the domains of the Fab, using REFMAC5 (The CCP4 Suite: Programs for Protein Crystallography. Acta Cryst. D50, 760-763 (1990).

BIAcore binding studies. Surface plasmon resonance binding experiments, performed using a BIAcore 3000 instrument, were performed in 10 mM Hepes buffer, pH 8.0, that contained 150 mM NaCl, 3 mM EDTA, and 0.005% Tween 20 (HBS-EP8) at 25° C. EGF-agonists (200 μg/ml) were coupled to a CM5 BIAcore sensor chip using standard amine coupling. Optimal coupling was obtained in 10 mM sodium acetate at pH 4.0 for EGF and TGFα and at pH 6.0 for HB-EGF. Binding of sEGFR to these immobilized ligands was performed and analyzed exactly as described in Ferguson, K. M. et al., 2000 (FIG. 4c). The Fab fragment of cetuximab was coupled to a separate sensor chip using amine coupling. The Fab was diluted to 50 μg/ml in 10 mM sodium acetate at pH 5.5 and passed over the activated surface for 5 minute at a flow rate 10 μl/minute. The binding of sEGFR to this surface was determined exactly as for sEGFR binding to immobilized EGF (Ferguson, K. M. et al., 2000) with the following modifications; a long contact time was used (10 μl/min for 20 minutes; 200 μl injection) to ensure that equilibrium was reached in binding of sEGFR to the surface even at low concentration, the surface was regenerated between data points with two 5 μl injections of 10 mM glycine, 1M NaCl (pH 3.0) to rapidly remove residual bound sEGFR. This regeneration does not impair the binding of sEGFR to the Fab; the observed response for a control sEGFR sample is constant over multiple cycles of binding and regeneration (FIG. 4(a)).

The effect of added Fab upon the binding of sEGFR to immobilized ligand was determined using the same EGF-agonist chip described above. A series of samples were prepared that contained 600 nM sEGFR and increasing amounts of Fab. The fraction of the maximal response in the absence of added Fab is plotted for each ligand (FIG. 4(b)).

EGFR:Cetuximab Fab Interface. The following amino acids are involved in direct hydrogen bonds with the Fab (3.25 Å cut-off, calculated using the program CONTACT (CCP4)):

| | Cetuximab* | Cetuximab* | |
|---|---|---|---|
| sEGFR | Light Chain | Heavy Chain | Type |
| Ser 468 | Asn 91 | | Main chain - main chain |
| Asn 469 | Trp 94 | | Main chain - main chain |
| Arg 353 | | Gly 54 | Side chain - main chain |
| Gln 384 | | Tyr 102 | Side chain - side chain |
| Gln 408 | | Tyr 102 | Side chain - side chain |
| Ser 418 | | Trp 52 | Side chain - side chain |
| Ser 440 | | Tyr 102 | Side chain - main chain |
| Lys 465 | | Asp 103 | Side chain - side chain |

*amino acids in the Fab are numbered in a simple sequential manner.

Additional amino acids that are close (4 Å cut-off) are shown on the following sequence.

| 310 320 330 340 350 360 | |
|---|---|
| B1 RKVCNGIGIG EFKDSLSINA TNIKHFKNCT SISGDLHILP VAFRGDSFTH TPPLDPQELD | |
| 370 380 390 400 410 420 | |
| ILKTVKEITG FLLIQAWPEN RTDLHAFENL EIIRGRTKQH GQFSLAVVSL NITSLGLRSL | |
| 430 440 450 460 470 | |
| KEISDGDVII SGNKNLCYAN TINWKKLFGT SGQKTKIISN RGENSCKA | |
| Bold | Fab Heavy chain |
| Underlined and Italic | Fab Light chain |
| Italic | Both chains of Fab |

The binding site for cetuximab Fab is partially over-lapping with the ligand binding site. The following amino acids are involved in contact to TGFα or EGF, as reported by Garrett et al. and Ogiso et al.

| 310 320 330 340 350 360 | |
|---|---|
| B1 RKVCNGIGIG EFKDSLSINA TNIKHFKNCT SISGDLHILP VAFRGDSFTH TPPLDPQELD | |
| 370 380 390 400 410 420 | |
| ILKTVKEITG FLLIQAWPEN RTDLHAFENL EIIRGRTKQH GQFSLAVVSL NITSLGLRSL | |
| 430 440 450 460 470 | |
| KEISDGDVII SGNKNLCYAN TINWKKLFGT SGQKTKIISN RGENSCKA | |
| Underlined | EGF/TGFα |

TABLE 1
Data collection and refinement statistics
Data Collection Statistics^{a}

| Space group | P2₁ |
|---|---|
| Unique cell dimensions | a = 77.8 Å, b = 70.9 Å, |
| | c = 147.1 Å; β = 102.5° |
| X-ray source | CHESS A1 |
| Resolution limit | 2.8 Å |
| Observed/unique | 1411,255/38,478 |
| Completeness | 99.8 (90.6) |
| R_{sym}^{b} | 0.03 (0.33) |
| <I/σ> | 17 (5.6) |

Refinement Statistics

| | |
|---|---|
| Resolution limits | 500-2.8 Å |
| No. of reflections/no. | 38098/1900 |
| test set | |
| R factor (R_{free})^{c} | 0.22 (0.27) |

Model

| | |
|---|---|
| Protein | sEGFR - aa 1-614 |
| | Fab: Light chain; aa 1-211, |
| | Heavy Chain; aa 1-220, |
| | 25 saccharide units |
| Total number of atoms | 8131 |
| RMSD bond lengths (Å) | 0.028 Å |
| RMSD bond angles (°) | 2.63° |

^{a}Numbers in parentheses refer to last resolution shell
^{b}R_{sym} = Σ|Iₕ − <Iₕ>|/ΣIₕ, where <Iₕ> = average intensity over symmetry equivalent measurements
^{c}R factor = Σ|Fₒ − F_{c}|/ΣFₒ, where summation is over data used in the refinement; R_{free} includes only 5% of the data excluded from the refinement

TABLE 2

| HEADER | | HORMONE/GROWTH FACTOR RECEPTOR |
|---|---|---|
| TITLE | | STRUCTURE OF THE EXTRACELLULAR DOMAIN OF HUMAN EPIDERMAL |
| TITLE | 2 | GROWTH FACTOR (EGF) RECEPTOR IN AN COMPLEX WITH IMC-C225 |
| TITLE | 3 | (CETUXIMBA/ERBITUX). |

| | |
|---|---|
| COMPND | MOL_ID: 1; |

| COMPND | 2 | MOLECULE: EPIDERMAL GROWTH FACTOR RECEPTOR; | |
|---|---|---|---|
| COMPND | 3 | CHAIN: A; | |
| COMPND | 4 | FRAGMENT: EXTRACELLULAR DOMAIN; | |
| COMPND | 5 | ENGINEERED: YES; | |
| COMPND | 6 | MOL_ID: 2; | |
| COMPND | 7 | MOLECULE: FAB FRAGMENT FROM CETUXIMAB; | |
| COMPND | 8 | CHAIN: C; | |
| COMPND | 9 | CHAIN: D; | |
| COMPND | 10 | SYNONYM: IMC-C225, ERBITUX; | |
| COMPND | 11 | ENGINEERED: YES | |
| SOURCE | | MOL_ID: 1; | |
| SOURCE | 2 | ORGANISM_SCIENTIFIC: HOMO SAPIENS; | |
| SOURCE | 3 | ORGANISM_COMMON: HUMAN; | |
| SOURCE | 4 | EXPRESSION_SYSTEM: SPODOPTERA FRUGIPERDA; | |
| SOURCE | 5 | EXPRESSION_SYSTEM_COMMON: FALL ARMYWORM; | |
| SOURCE | 6 | EXPRESSION_SYSTEM_STRAIN: VIRUS; | |
| SOURCE | 7 | EXPRESSION_SYSTEM_VECTOR_TYPE: BACULOVIRUS; | |
| SOURCE | 8 | MOL_ID: 2; | |
| SOURCE | | PROVIDED BY IMCLONE INC. AS FAB FRAGMENT | |
| EXPDTA | | X-RAY DIFFRACTION | |
| REMARK | 1 | | |
| REMARK | 2 | RESOLUTION. 2.80 ANGSTROMS. | |
| REMARK | 3 | REFINEMENT. | |
| REMARK | 3 | PROGRAM | : REFMAC 5.1.24 |
| REMARK | 3 | AUTHORS | : MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.81 |
| REMARK | 3 | RESOLUTION RANGE LOW | (ANGSTROMS) | : 50.00 |
| REMARK | 3 | DATA CUTOFF | (SIGMA(F)) | : NONE |

| | | | | |
|---|---|---|---|---|
| REMARK | 3 | COMPLETENESS FOR RANGE | (%) | : 99.44 |
| REMARK | 3 | NUMBER OF REFLECTIONS | | : 36547 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.22623 |
| REMARK | 3 | R VALUE | (WORKING SET) | : 0.22396 |

| REMARK | 3 | FREE R VALUE | | : 0.26877 |
|---|---|---|---|---|
| REMARK | 3 | FREE R VALUE TEST SET SIZE | (%) | : 5.0 |

| | | | |
|---|---|---|---|
| REMARK | 3 | FREE R VALUE TEST SET COUNT | : 1931 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 2.805 |
| REMARK | 3 | BIN RESOLUTION RANGE LOW | | : 2.878 |
| REMARK | 3 | REFLECTION IN BIN | (WORKING SET) | : 2605 |
| REMARK | 3 | BIN R VALUE | (WORKING SET) | : 0.335 |

| | | | |
|---|---|---|---|
| REMARK | 3 | BIN FREE R VALUE SET COUNT | : 141 |
| REMARK | 3 | BIN FREE R VALUE | : 0.381 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 8131 |
| REMARK | 3 | | |
| REMARK | 3 | B VALUES. | |

| | | | | |
|---|---|---|---|---|
| REMARK | 3 | FROM WILSON PLOT | (A**2) : | NULL |
| REMARK | 3 | MEAN B VALUE | (OVERALL, A**2) : | 7.403 |

| | | |
|---|---|---|
| REMARK | 3 | OVERALL ANISOTROPIC B VALUE. |

| | | | |
|---|---|---|---|
| REMARK | 3 | B11 (A**2) : | 1.07 |
| REMARK | 3 | B22 (A**2) : | 0.32 |
| REMARK | 3 | B33 (A**2) : | −1.83 |
| REMARK | 3 | B12 (A**2) : | 0.00 |
| REMARK | 3 | B13 (A**2) : | −1.01 |
| REMARK | 3 | B23 (A**2) : | 0.00 |
| REMARK | 3 | | |

| REMARK | 3 | ESTIMATED OVERALL COORDINATE ERROR. | | |
|---|---|---|---|---|
| REMARK | 3 | ESU BASED ON R VALUE | (A): | 0.910 |
| REMARK | 3 | ESU BASED ON FREE R VALUE | (A): | 0.362 |
| REMARK | 3 | ESU BASED ON MAXIMUM LIKELIHOOD | (A): | 0.295 |
| REMARK | 3 | ESU FOR B VALUES BASED ON MAXIMUM LIKELIHOOD | (A**2): | 15.477 |
| REMARK | 3 | | | |

| | | | |
|---|---|---|---|
| REMARK | 3 | CORRELATION COEFFICIENTS. | |
| REMARK | 3 | CORRELATION COEFFICIENT FO-FC | : 0.925 |
| REMARK | 3 | CORRELATION COEFFICIENT FO-FC FREE | : 0.888 |
| REMARK | 3 | | |

| | | | | | | |
|---|---|---|---|---|---|---|
| REMARK | 3 | RMS DEVIATIONS FROM IDEAL VALUES | | COUNT | RMS | WEIGHT |
| REMARK | 3 | BOND LENGTHS REFINED ATOMS | (A): | 8349 ; | 0.028 ; | 0.021 |
| REMARK | 3 | BOND LENGTHS OTHERS | (A): | 7109 ; | 0.002 ; | 0.020 |
| REMARK | 3 | BOND ANGLES REFINED ATOMS | (DEGREES): | 11411 ; | 2.623 ; | 1.993 |
| REMARK | 3 | BOND ANGLES OTHERS | (DEGREES): | 16529 ; | 1.130 ; | 3.000 |
| REMARK | 3 | TORSION ANGLES, PERIOD 1 | (DEGREES): | 1042 ; | 9.222 ; | 5.000 |
| REMARK | 3 | CHIRAL-CENTER RESTRAINTS | (A**3): | 1349 ; | 0.132 ; | 0.200 |
| REMARK | 3 | GENERAL PLANES REFINED ATOMS | (A): | 9136 ; | 0.009 ; | 0.020 |
| REMARK | 3 | GENERAL PLANES OTHERS | (A): | 1532 ; | 0.007 ; | 0.020 |
| REMARK | 3 | NON-BONDED CONTACTS REFINED ATOMS | (A): | 1896 ; | 0.255 ; | 0.200 |
| REMARK | 3 | NON-BONDED CONTACTS OTHERS | (A): | 8610 ; | 0.250 ; | 0.200 |
| REMARK | 3 | NON-BONDED TORSION OTHERS | (A): | 5383 ; | 0.106 ; | 0.200 |
| REMARK | 3 | H-BOND (X . . . Y) REFINED ATOMS | (A): | 146 ; | 0.207 ; | 0.200 |
| REMARK | 3 | SYMMETRY VDW REFINED ATOMS | (A): | 11 ; | 0.149 ; | 0.200 |
| REMARK | 3 | SYMMETRY VDW OTHERS | (A): | 51 ; | 0.288 ; | 0.200 |
| REMARK | 3 | SYMMETRY H-BOND REFINED ATOMS | (A): | 3 ; | 0.346 ; | 0.200 |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2): | 5197 ; | 0.717 ; | 1.500 |
| REMARK | 3 | MAIN-CHAIN ANGLE REFINED ATOMS | (A**2): | 8355 ; | 1.289 ; | 2.000 |
| REMARK | 3 | SIDE-CHAIN BOND REFINED ATOMS | (A**2): | 3152 ; | 2.076 ; | 3.000 |
| REMARK | 3 | SIDE-CHAIN ANGLE REFINED ATOMS | (A**2): | 3056 ; | 3.116 ; | 4.500 |
| REMARK | 3 | | | | | |
| REMARK | 3 | NCS RESTRAINTS STATISTICS | | | | |
| REMARK | 3 | NUMBER OF NCS GROUPS: NULL | | | | |
| REMARK | 3 | | | | | |
| REMARK | 3 | BULK SOLVENT MODELLING. | | | | |
| REMARK | 3 | METHOD USED: BABINET MODEL WITH MASK | | | | |
| REMARK | 3 | PARAMETERS FOR MASK CALCULATION | | | | |

| | | | | |
|---|---|---|---|---|
| REMARK | 3 | VDW PROBE RADIUS | : | 1.40 |
| REMARK | 3 | ION PROBE RADIUS | : | 0.80 |
| REMARK | 3 | SHRINKAGE RADIUS | : | 0.80 |
| REMARK | 3 | | | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |

| | | | | | | | | | |
|---|---|---|---|---|---|---|---|---|---|
| LINK | C1 | NAG | E3281 | 1.439 | ND2 | ASN | A | 328 | NAG-ASN |
| LINK | C1 | NAG | E3371 | 1.439 | ND2 | ASN | A | 337 | NAG-ASN |
| LINK | C1 | NAG | E3891 | 1.439 | ND2 | ASN | A | 389 | NAG-ASN |
| LINK | C1 | NAG | E5441 | 1.439 | ND2 | ASN | A | 544 | NAG-ASN |
| LINK | C1 | NAG | E5791 | 1.439 | ND2 | ASN | A | 579 | NAG-ASN |
| LINK | C1 | NAG | E 881 | 1.439 | ND2 | ASN | D | 88 | NAG-ASN |
| LINK | O3 | NAG | E 321 | | N2 | NAG | E | 322 | NAG-NAG |
| LINK | O3 | NAG | E 321 | | C7 | NAG | E | 322 | NAG-NAG1 |
| LINK | O3 | NAG | E 321 | | O7 | NAG | E | 322 | NAG-NAG2 |
| LINK | O2 | MAN | E 324 | 1.080 | O2 | MAN | E | 323 | MAN-MAN |

| | | | | | | | | |
|---|---|---|---|---|---|---|---|---|
| CISPEP | 1 | SER | C | 7 | PRO | C | 8 | 0.00 |
| CISPEP | 2 | TYR | C | 140 | PRO | C | 141 | 0.00 |
| CISPEP | 3 | PHE | D | 152 | PRO | D | 153 | 0.00 |
| CISPEP | 4 | GLU | D | 154 | PRO | D | 155 | 0.00 |

| | | | | | |
|---|---|---|---|---|---|
| LINK | NAG | E3281 | NAG | E3282 | BETA1-4 |
| LINK | NAG | E3282 | MAN | E3283 | BETA1-4 |
| LINK | MAN | E3283 | MAN | E3284 | BETA1-6 |
| LINK | MAN | E3284 | MAN | E3287 | ALPHA1-3 |
| LINK | MAN | E3287 | MAN | E3288 | BETA1-2 |
| LINK | MAN | E3283 | MAN | E3285 | ALPHA1-3 |
| LINK | MAN | E3285 | MAN | E3286 | BETA1-2 |
| LINK | MAN | E3284 | MAN | E3289 | ALPHA1-6 |
| LINK | NAG | E3371 | NAG | E3372 | ALPHA1-4 |
| LINK | NAG | E3372 | MAN | E3373 | ALPHA1-4 |
| LINK | MAN | E3373 | MAN | E3374 | BETA1-3 |
| LINK | NAG | E4201 | NAG | E4202 | ALPHA1-4 |
| LINK | NAG | E4202 | MAN | E4203 | ALPHA1-4 |

| | | | | | | | |
|---|---|---|---|---|---|---|---|
| SSBOND | 1 | CYS | A | 34 | CYS | A | 7 |
| SSBOND | 2 | CYS | A | 163 | CYS | A | 133 |
| SSBOND | 3 | CYS | A | 175 | CYS | A | 166 |
| SSBOND | 4 | CYS | A | 183 | CYS | A | 170 |
| SSBOND | 5 | CYS | A | 207 | CYS | A | 195 |
| SSBOND | 6 | CYS | A | 216 | CYS | A | 208 |
| SSBOND | 7 | CYS | A | 224 | CYS | A | 212 |
| SSBOND | 8 | CYS | A | 236 | CYS | A | 227 |
| SSBOND | 9 | CYS | A | 283 | CYS | A | 271 |
| SSBOND | 10 | CYS | A | 302 | CYS | A | 287 |
| SSBOND | 11 | CYS | A | 309 | CYS | A | 305 |
| SSBOND | 12 | CYS | A | 338 | CYS | A | 313 |
| SSBOND | 13 | CYS | A | 499 | CYS | A | 486 |
| SSBOND | 14 | CYS | A | 511 | CYS | A | 502 |
| SSBOND | 15 | CYS | A | 531 | CYS | A | 515 |
| SSBOND | 16 | CYS | A | 547 | CYS | A | 534 |
| SSBOND | 17 | CYS | A | 555 | CYS | A | 538 |
| SSBOND | 18 | CYS | A | 567 | CYS | A | 558 |
| SSBOND | 19 | CYS | A | 593 | CYS | A | 571 |
| SSBOND | 20 | CYS | A | 604 | CYS | A | 596 |
| SSBOND | 21 | CYS | A | 612 | CYS | A | 600 |
| SSBOND | 22 | CYS | A | 446 | CYS | A | 475 |
| SSBOND | 23 | CYS | A | 482 | CYS | A | 491 |
| SSBOND | 24 | CYS | A | 199 | CYS | A | 191 |
| SSBOND | 25 | CYS | C | 88 | CYS | C | 23 |
| SSBOND | 26 | CYS | C | 194 | CYS | C | 134 |
| SSBOND | 27 | CYS | D | 95 | CYS | D | 22 |
| SSBOND | 28 | CYS | D | 202 | CYS | D | 146 |

| | | | | | |
|---|---|---|---|---|---|
| MODRES | NAG | E | 321 | NAG-b-D | RENAME |
| MODRES | NAG | E | 322 | NAG-b-D | RENAME |
| MODRES | MAN | E | 323 | MAN-b-D | RENAME |
| MODRES | MAN | E | 324 | MAN-b-D | RENAME |
| MODRES | NAG | E | 3281 | NAG-b-D | RENAME |
| MODRES | NAG | E | 3282 | NAG-b-D | RENAME |
| MODRES | MAN | E | 3283 | MAN-b-D | RENAME |
| MODRES | MAN | E | 3284 | MAN-b-D | RENAME |
| MODRES | MAN | E | 3287 | MAN-a-D | RENAME |
| MODRES | MAN | E | 3288 | MAN-b-D | RENAME |
| MODRES | MAN | E | 3285 | MAN-a-D | RENAME |
| MODRES | MAN | E | 3286 | MAN-b-D | RENAME |
| MODRES | MAN | E | 3289 | MAN-a-D | RENAME |
| MODRES | NAG | E | 3371 | NAG-b-D | RENAME |
| MODRES | NAG | E | 3372 | NAG-a-D | RENAME |
| MODRES | MAN | E | 3373 | MAN-a-D | RENAME |
| MODRES | MAN | E | 3374 | MAN-b-D | RENAME |
| MODRES | NAG | E | 3891 | NAG-b-D | RENAME |
| MODRES | NAG | E | 3892 | NAG-b-D | RENAME |
| MODRES | NAG | E | 4201 | NAG-b-D | RENAME |
| MODRES | NAG | E | 4202 | NAG-a-D | RENAME |
| MODRES | MAN | E | 4203 | MAN-a-D | RENAME |
| MODRES | NAG | E | 5041 | NAG-b-D | RENAME |
| MODRES | NAG | E | 5441 | NAG-b-D | RENAME |
| MODRES | NAG | E | 5791 | NAG-b-D | RENAME |
| MODRES | NAG | E | 881 | NAG-b-D | RENAME |

| | | | | | | | |
|---|---|---|---|---|---|---|---|
| CRYST1 | 77.823 | 70.861 | 147.122 | 90.00 | 102.48 | 90.00 | P 1 21 1 |

| | | | | |
|---|---|---|---|---|
| SCALE1 | 0.012850 | 0.000000 | 0.002844 | 0.00000 |
| SCALE2 | 0.000000 | 0.014112 | 0.000000 | 0.00000 |
| SCALE3 | 0.000000 | 0.000000 | 0.006962 | 0.00000 |

| | | | | | | | | | | | |
|---|---|---|---|---|---|---|---|---|---|---|---|
| ATOM | 1 | N | LEU | A | 1 | 13.360 | 2.249 | 11.251 | 1.00 | 100.92 | N |
| ATOM | 3 | CA | LEU | A | 1 | 12.773 | 1.470 | 12.398 | 1.00 | 105.53 | C |
| ATOM | 5 | CB | LEU | A | 1 | 11.965 | 0.248 | 11.865 | 1.00 | 108.08 | C |
| ATOM | 8 | CG | LEU | A | 1 | 10.656 | −0.207 | 12.561 | 1.00 | 114.07 | C |
| ATOM | 10 | CD1 | LEU | A | 1 | 9.579 | 0.912 | 12.805 | 1.00 | 118.27 | C |
| ATOM | 14 | CD2 | LEU | A | 1 | 10.023 | −1.358 | 11.772 | 1.00 | 115.39 | C |
| ATOM | 18 | C | LEU | A | 1 | 13.923 | 1.032 | 13.354 | 1.00 | 103.08 | C |
| ATOM | 19 | O | LEU | A | 1 | 13.913 | 1.330 | 14.572 | 1.00 | 105.39 | O |
| ATOM | 22 | N | GLU | A | 2 | 14.904 | 0.333 | 12.772 | 1.00 | 98.75 | N |
| ATOM | 24 | CA | GLU | A | 2 | 16.143 | −0.038 | 13.449 | 1.00 | 95.06 | C |
| ATOM | 26 | CB | GLU | A | 2 | 16.981 | −0.917 | 12.518 | 1.00 | 90.85 | C |
| ATOM | 29 | CG | GLU | A | 2 | 18.025 | −1.714 | 13.253 | 1.00 | 89.57 | C |
| ATOM | 32 | CD | GLU | A | 2 | 19.160 | −2.205 | 12.358 | 1.00 | 88.28 | C |
| ATOM | 33 | OE1 | GLU | A | 2 | 19.720 | −1.403 | 11.495 | 1.00 | 84.21 | O |
| ATOM | 34 | OE2 | GLU | A | 2 | 19.493 | −3.427 | 12.562 | 1.00 | 89.66 | O |
| ATOM | 35 | C | GLU | A | 2 | 16.965 | 1.207 | 13.819 | 1.00 | 92.58 | C |
| ATOM | 36 | O | GLU | A | 2 | 17.201 | 2.090 | 12.955 | 1.00 | 90.00 | O |
| ATOM | 37 | N | GLU | A | 3 | 17.396 | 1.261 | 15.096 | 1.00 | 93.29 | N |
| ATOM | 39 | CA | GLU | A | 3 | 18.416 | 2.250 | 15.593 | 1.00 | 90.23 | C |
| ATOM | 41 | CB | GLU | A | 3 | 18.082 | 2.630 | 17.081 | 1.00 | 94.21 | C |
| ATOM | 44 | CG | GLU | A | 3 | 16.599 | 3.021 | 17.380 | 1.00 | 100.00 | C |
| ATOM | 47 | CD | GLU | A | 3 | 16.165 | 2.910 | 18.872 | 1.00 | 105.60 | C |
| ATOM | 48 | OE1 | GLU | A | 3 | 16.983 | 2.478 | 19.722 | 1.00 | 104.83 | O |
| ATOM | 49 | OE2 | GLU | A | 3 | 14.991 | 3.253 | 19.229 | 1.00 | 109.98 | O |
| ATOM | 50 | C | GLU | A | 3 | 19.925 | 1.714 | 15.412 | 1.00 | 84.70 | C |
| ATOM | 51 | O | GLU | A | 3 | 20.405 | 0.936 | 16.274 | 1.00 | 85.80 | O |
| ATOM | 52 | N | LYS | A | 4 | 20.618 | 2.086 | 14.305 | 1.00 | 79.39 | N |
| ATOM | 54 | CA | LYS | A | 4 | 21.994 | 1.608 | 13.949 | 1.00 | 74.36 | C |
| ATOM | 56 | CB | LYS | A | 4 | 22.472 | 2.211 | 12.626 | 1.00 | 70.99 | C |
| ATOM | 59 | CG | LYS | A | 4 | 21.654 | 1.834 | 11.436 | 1.00 | 73.21 | C |
| ATOM | 62 | CD | LYS | A | 4 | 22.526 | 1.263 | 10.289 | 1.00 | 71.25 | C |
| ATOM | 65 | CE | LYS | A | 4 | 21.667 | 0.401 | 9.254 | 1.00 | 75.31 | C |
| ATOM | 68 | NZ | LYS | A | 4 | 21.491 | 1.128 | 7.883 | 1.00 | 76.46 | N |
| ATOM | 72 | C | LYS | A | 4 | 23.090 | 2.043 | 14.898 | 1.00 | 72.83 | C |
| ATOM | 73 | O | LYS | A | 4 | 23.169 | 3.249 | 15.216 | 1.00 | 73.52 | O |
| ATOM | 74 | N | LYS | A | 5 | 24.009 | 1.129 | 15.248 | 1.00 | 70.16 | N |
| ATOM | 76 | CA | LYS | A | 5 | 24.974 | 1.410 | 16.328 | 1.00 | 68.70 | C |
| ATOM | 78 | CB | LYS | A | 5 | 25.338 | 0.124 | 17.092 | 1.00 | 69.57 | C |
| ATOM | 81 | CG | LYS | A | 5 | 24.052 | −0.768 | 17.413 | 1.00 | 75.20 | C |
| ATOM | 84 | CD | LYS | A | 5 | 23.641 | −0.893 | 18.917 | 1.00 | 79.29 | C |
| ATOM | 87 | CE | LYS | A | 5 | 22.196 | −0.390 | 19.171 | 1.00 | 83.67 | C |
| ATOM | 90 | NZ | LYS | A | 5 | 21.488 | −1.198 | 20.194 | 1.00 | 91.94 | N |
| ATOM | 94 | C | LYS | A | 5 | 26.206 | 2.153 | 15.828 | 1.00 | 64.30 | C |
| ATOM | 95 | O | LYS | A | 5 | 26.682 | 1.926 | 14.749 | 1.00 | 61.85 | O |
| ATOM | 96 | N | VAL | A | 6 | 26.724 | 3.047 | 16.647 | 1.00 | 64.34 | N |
| ATOM | 98 | CA | VAL | A | 6 | 27.596 | 4.114 | 16.203 | 1.00 | 61.78 | C |
| ATOM | 100 | CB | VAL | A | 6 | 26.890 | 5.440 | 16.468 | 1.00 | 63.52 | C |
| ATOM | 102 | CG1 | VAL | A | 6 | 27.642 | 6.564 | 15.784 | 1.00 | 61.56 | C |
| ATOM | 106 | CG2 | VAL | A | 6 | 25.473 | 5.382 | 15.997 | 1.00 | 66.36 | C |
| ATOM | 110 | C | VAL | A | 6 | 28.801 | 4.255 | 17.058 | 1.00 | 61.00 | C |
| ATOM | 111 | O | VAL | A | 6 | 28.640 | 4.254 | 18.203 | 1.00 | 63.87 | O |
| ATOM | 112 | N | CYS | A | 7 | 29.982 | 4.525 | 16.546 | 1.00 | 58.45 | N |
| ATOM | 114 | CA | CYS | A | 7 | 31.094 | 4.807 | 17.432 | 1.00 | 59.36 | C |
| ATOM | 116 | CB | CYS | A | 7 | 31.943 | 3.561 | 17.567 | 1.00 | 57.65 | C |
| ATOM | 119 | SG | CYS | A | 7 | 32.512 | 2.903 | 16.006 | 1.00 | 58.73 | S |
| ATOM | 120 | C | CYS | A | 7 | 31.926 | 5.983 | 16.972 | 1.00 | 58.39 | C |
| ATOM | 121 | O | CYS | A | 7 | 31.955 | 6.264 | 15.829 | 1.00 | 57.51 | O |
| ATOM | 122 | N | GLN | A | 8 | 32.585 | 6.702 | 17.874 | 1.00 | 61.38 | N |
| ATOM | 124 | CA | GLN | A | 8 | 33.635 | 7.683 | 17.502 | 1.00 | 61.54 | C |
| ATOM | 126 | CB | GLN | A | 8 | 34.054 | 8.482 | 18.741 | 1.00 | 63.20 | C |
| ATOM | 129 | CG | GLN | A | 8 | 32.927 | 9.373 | 19.254 | 1.00 | 67.39 | C |
| ATOM | 132 | CD | GLN | A | 8 | 32.574 | 10.563 | 18.334 | 1.00 | 70.24 | C |
| ATOM | 133 | OE1 | GLN | A | 8 | 33.273 | 10.818 | 17.335 | 1.00 | 70.24 | O |
| ATOM | 134 | NE2 | GLN | A | 8 | 31.479 | 11.300 | 18.673 | 1.00 | 75.33 | N |
| ATOM | 137 | C | GLN | A | 8 | 34.857 | 6.935 | 16.978 | 1.00 | 60.56 | C |
| ATOM | 138 | O | GLN | A | 8 | 35.205 | 5.857 | 17.449 | 1.00 | 63.77 | O |
| ATOM | 139 | N | GLY | A | 9 | 35.579 | 7.394 | 16.026 | 1.00 | 58.91 | N |
| ATOM | 141 | CA | GLY | A | 9 | 36.720 | 6.527 | 15.726 | 1.00 | 57.92 | C |
| ATOM | 144 | C | GLY | A | 9 | 38.036 | 7.249 | 15.990 | 1.00 | 60.20 | C |
| ATOM | 145 | O | GLY | A | 9 | 38.259 | 8.517 | 15.590 | 1.00 | 64.00 | O |
| ATOM | 146 | N | THR | A | 10 | 38.926 | 6.543 | 16.652 | 1.00 | 59.60 | N |
| ATOM | 148 | CA | THR | A | 10 | 40.369 | 6.891 | 16.558 | 1.00 | 60.20 | C |
| ATOM | 150 | CB | THR | A | 10 | 41.078 | 5.669 | 16.340 | 1.00 | 59.09 | C |
| ATOM | 152 | OG1 | THR | A | 10 | 40.256 | 4.658 | 16.885 | 1.00 | 66.90 | O |
| ATOM | 154 | CG2 | THR | A | 10 | 42.281 | 5.634 | 17.296 | 1.00 | 61.60 | C |
| ATOM | 158 | C | THR | A | 10 | 40.998 | 7.884 | 15.538 | 1.00 | 57.46 | C |
| ATOM | 159 | O | THR | A | 10 | 40.657 | 7.865 | 14.372 | 1.00 | 55.26 | O |
| ATOM | 160 | N | SER | A | 11 | 41.945 | 8.692 | 16.037 | 1.00 | 57.21 | N |
| ATOM | 162 | CA | SER | A | 11 | 42.838 | 9.479 | 15.220 | 1.00 | 56.01 | C |
| ATOM | 164 | CB | SER | A | 11 | 42.438 | 10.930 | 15.303 | 1.00 | 58.10 | C |
| ATOM | 167 | OG | SER | A | 11 | 41.073 | 11.006 | 15.003 | 1.00 | 58.88 | O |
| ATOM | 169 | C | SER | A | 11 | 44.237 | 9.330 | 15.720 | 1.00 | 56.12 | C |
| ATOM | 170 | O | SER | A | 11 | 44.916 | 10.266 | 16.008 | 1.00 | 56.39 | O |
| ATOM | 171 | N | ASN | A | 12 | 44.687 | 8.118 | 15.834 | 1.00 | 55.44 | N |
| ATOM | 173 | CA | ASN | A | 12 | 46.051 | 7.926 | 16.295 | 1.00 | 58.03 | C |
| ATOM | 175 | CB | ASN | A | 12 | 46.089 | 6.865 | 17.413 | 1.00 | 58.48 | C |
| ATOM | 178 | CG | ASN | A | 12 | 44.886 | 6.955 | 18.346 | 1.00 | 59.19 | C |
| ATOM | 179 | OD1 | ASN | A | 12 | 44.347 | 8.062 | 18.563 | 1.00 | 59.90 | O |
| ATOM | 180 | ND2 | ASN | A | 12 | 44.426 | 5.784 | 18.880 | 1.00 | 56.12 | N |
| ATOM | 183 | C | ASN | A | 12 | 47.084 | 7.624 | 15.172 | 1.00 | 57.48 | C |
| ATOM | 184 | O | ASN | A | 12 | 48.285 | 7.497 | 15.459 | 1.00 | 60.22 | O |
| ATOM | 185 | N | LYS | A | 13 | 46.655 | 7.497 | 13.923 | 1.00 | 54.67 | N |
| ATOM | 187 | CA | LYS | A | 13 | 47.614 | 7.192 | 12.876 | 1.00 | 55.14 | C |
| ATOM | 189 | CB | LYS | A | 13 | 48.381 | 8.476 | 12.501 | 1.00 | 57.96 | C |
| ATOM | 192 | CG | LYS | A | 13 | 47.420 | 9.634 | 12.179 | 1.00 | 57.91 | C |
| ATOM | 195 | CD | LYS | A | 13 | 48.048 | 10.642 | 11.298 | 1.00 | 60.41 | C |
| ATOM | 198 | CE | LYS | A | 13 | 47.075 | 11.808 | 10.983 | 1.00 | 63.26 | C |
| ATOM | 201 | NZ | LYS | A | 13 | 47.797 | 13.059 | 10.472 | 1.00 | 67.44 | N |
| ATOM | 205 | C | LYS | A | 13 | 48.582 | 6.093 | 13.297 | 1.00 | 56.16 | C |
| ATOM | 206 | O | LYS | A | 13 | 48.171 | 4.995 | 13.766 | 1.00 | 56.29 | O |
| ATOM | 207 | N | LEU | A | 14 | 49.880 | 6.360 | 13.196 | 1.00 | 57.76 | N |
| ATOM | 209 | CA | LEU | A | 14 | 50.813 | 5.266 | 13.406 | 1.00 | 57.81 | C |
| ATOM | 211 | CB | LEU | A | 14 | 51.985 | 5.375 | 12.424 | 1.00 | 58.72 | C |
| ATOM | 214 | CG | LEU | A | 14 | 51.524 | 5.396 | 10.955 | 1.00 | 56.05 | C |
| ATOM | 216 | CD1 | LEU | A | 14 | 52.683 | 5.353 | 9.975 | 1.00 | 57.66 | C |
| ATOM | 220 | CD2 | LEU | A | 14 | 50.536 | 4.287 | 10.645 | 1.00 | 53.10 | C |
| ATOM | 224 | C | LEU | A | 14 | 51.243 | 5.154 | 14.855 | 1.00 | 60.01 | C |
| ATOM | 225 | O | LEU | A | 14 | 52.038 | 4.261 | 15.187 | 1.00 | 61.31 | O |
| ATOM | 226 | N | THR | A | 15 | 50.731 | 6.037 | 15.716 | 1.00 | 60.49 | N |
| ATOM | 228 | CA | THR | A | 15 | 50.925 | 5.854 | 17.142 | 1.00 | 63.51 | C |
| ATOM | 230 | CB | THR | A | 15 | 50.300 | 6.950 | 17.944 | 1.00 | 64.28 | C |
| ATOM | 232 | OG1 | THR | A | 15 | 50.825 | 8.199 | 17.543 | 1.00 | 65.35 | O |
| ATOM | 234 | CG2 | THR | A | 15 | 50.791 | 6.867 | 19.429 | 1.00 | 69.05 | C |
| ATOM | 238 | C | THR | A | 15 | 50.397 | 4.495 | 17.648 | 1.00 | 63.38 | C |
| ATOM | 239 | O | THR | A | 15 | 49.322 | 4.011 | 17.232 | 1.00 | 61.95 | O |
| ATOM | 240 | N | GLN | A | 16 | 51.190 | 3.874 | 18.517 | 1.00 | 66.09 | N |
| ATOM | 242 | CA | GLN | A | 16 | 50.868 | 2.561 | 19.107 | 1.00 | 66.17 | C |
| ATOM | 244 | CB | GLN | A | 16 | 52.007 | 1.560 | 18.862 | 1.00 | 67.19 | C |
| ATOM | 247 | CG | GLN | A | 16 | 51.728 | 0.217 | 19.455 | 1.00 | 66.47 | C |
| ATOM | 250 | CD | GLN | A | 16 | 52.838 | −0.766 | 19.295 | 1.00 | 66.59 | C |
| ATOM | 251 | OE1 | GLN | A | 16 | 53.535 | −0.791 | 18.274 | 1.00 | 64.81 | O |
| ATOM | 252 | NE2 | GLN | A | 16 | 52.984 | −1.624 | 20.293 | 1.00 | 68.23 | N |
| ATOM | 255 | C | GLN | A | 16 | 50.617 | 2.737 | 20.632 | 1.00 | 69.21 | C |
| ATOM | 256 | O | GLN | A | 16 | 51.390 | 3.422 | 21.325 | 1.00 | 73.28 | O |
| ATOM | 257 | N | LEU | A | 17 | 49.533 | 2.144 | 21.148 | 1.00 | 67.72 | N |
| ATOM | 259 | CA | LEU | A | 17 | 49.033 | 2.455 | 22.486 | 1.00 | 69.49 | C |
| ATOM | 261 | CB | LEU | A | 17 | 47.504 | 2.620 | 22.478 | 1.00 | 67.31 | C |
| ATOM | 264 | CG | LEU | A | 17 | 46.923 | 3.591 | 21.437 | 1.00 | 64.82 | C |
| ATOM | 266 | CD1 | LEU | A | 17 | 45.375 | 3.565 | 21.397 | 1.00 | 62.29 | C |
| ATOM | 270 | CD2 | LEU | A | 17 | 47.487 | 5.032 | 21.639 | 1.00 | 66.62 | C |
| ATOM | 274 | C | LEU | A | 17 | 49.450 | 1.295 | 23.363 | 1.00 | 71.74 | C |
| ATOM | 275 | O | LEU | A | 17 | 48.609 | 0.497 | 23.794 | 1.00 | 71.04 | O |
| ATOM | 276 | N | GLY | A | 18 | 50.764 | 1.193 | 23.572 | 1.00 | 73.66 | N |
| ATOM | 278 | CA | GLY | A | 18 | 51.329 | 0.265 | 24.520 | 1.00 | 76.87 | C |
| ATOM | 281 | C | GLY | A | 18 | 51.609 | −1.038 | 23.830 | 1.00 | 75.65 | C |
| ATOM | 282 | O | GLY | A | 18 | 51.968 | −1.022 | 22.636 | 1.00 | 72.82 | O |
| ATOM | 283 | N | THR | A | 19 | 51.451 | −2.140 | 24.593 | 1.00 | 77.55 | N |
| ATOM | 285 | CA | THR | A | 19 | 51.581 | −3.516 | 24.099 | 1.00 | 76.35 | C |
| ATOM | 287 | CB | THR | A | 19 | 51.171 | −4.562 | 25.154 | 1.00 | 79.33 | C |
| ATOM | 289 | OG1 | THR | A | 19 | 51.943 | −4.422 | 26.343 | 1.00 | 83.29 | O |
| ATOM | 291 | CG2 | THR | A | 19 | 51.574 | −5.993 | 24.734 | 1.00 | 82.13 | C |
| ATOM | 295 | C | THR | A | 19 | 50.703 | −3.715 | 22.916 | 1.00 | 71.66 | C |
| ATOM | 296 | O | THR | A | 19 | 49.617 | −3.221 | 22.854 | 1.00 | 69.09 | O |
| ATOM | 297 | N | PHE | A | 20 | 51.211 | −4.445 | 21.952 | 1.00 | 71.13 | N |
| ATOM | 299 | CA | PHE | A | 20 | 50.404 | −4.916 | 20.873 | 1.00 | 68.02 | C |
| ATOM | 301 | CB | PHE | A | 20 | 51.089 | −6.099 | 20.219 | 1.00 | 69.47 | C |
| ATOM | 304 | CG | PHE | A | 20 | 52.300 | −5.752 | 19.353 | 1.00 | 70.89 | C |
| ATOM | 305 | CD1 | PHE | A | 20 | 53.413 | −6.599 | 19.350 | 1.00 | 74.82 | C |
| ATOM | 307 | CE1 | PHE | A | 20 | 54.500 | −6.334 | 18.562 | 1.00 | 76.46 | C |
| ATOM | 309 | CZ | PHE | A | 20 | 54.491 | −5.207 | 17.726 | 1.00 | 74.93 | C |
| ATOM | 311 | CE2 | PHE | A | 20 | 53.391 | −4.360 | 17.707 | 1.00 | 69.96 | C |
| ATOM | 313 | CD2 | PHE | A | 20 | 52.303 | −4.641 | 18.517 | 1.00 | 69.30 | C |
| ATOM | 315 | C | PHE | A | 20 | 49.083 | −5.409 | 21.417 | 1.00 | 67.44 | C |
| ATOM | 316 | O | PHE | A | 20 | 48.034 | −5.063 | 20.903 | 1.00 | 63.96 | O |
| ATOM | 317 | N | GLU | A | 21 | 49.124 | −6.245 | 22.449 | 1.00 | 71.20 | N |
| ATOM | 319 | CA | GLU | A | 21 | 47.872 | −6.628 | 23.125 | 1.00 | 71.94 | C |
| ATOM | 321 | CB | GLU | A | 21 | 48.020 | −7.643 | 24.239 | 1.00 | 75.53 | C |
| ATOM | 324 | CG | GLU | A | 21 | 46.614 | −8.017 | 24.681 | 1.00 | 77.19 | C |
| ATOM | 327 | CD | GLU | A | 21 | 46.443 | −9.456 | 25.095 | 1.00 | 84.24 | C |
| ATOM | 328 | OE1 | GLU | A | 21 | 46.760 | −9.761 | 26.300 | 1.00 | 86.94 | O |
| ATOM | 329 | OE2 | GLU | A | 21 | 45.962 | −10.251 | 24.217 | 1.00 | 85.07 | O |
| ATOM | 330 | C | GLU | A | 21 | 47.004 | −5.473 | 23.632 | 1.00 | 71.25 | C |
| ATOM | 331 | O | GLU | A | 21 | 45.855 | −5.383 | 23.189 | 1.00 | 69.23 | O |
| ATOM | 332 | N | ASP | A | 22 | 47.496 | −4.605 | 24.525 | 1.00 | 73.11 | N |
| ATOM | 334 | CA | ASP | A | 22 | 46.671 | −3.422 | 24.914 | 1.00 | 72.68 | C |
| ATOM | 336 | CB | ASP | A | 22 | 47.456 | −2.386 | 25.699 | 1.00 | 75.14 | C |
| ATOM | 339 | CG | ASP | A | 22 | 48.062 | −2.919 | 26.931 | 1.00 | 79.93 | C |
| ATOM | 340 | OD1 | ASP | A | 22 | 47.490 | −3.833 | 27.552 | 1.00 | 82.29 | O |
| ATOM | 341 | OD2 | ASP | A | 22 | 49.142 | −2.460 | 27.345 | 1.00 | 83.24 | O |
| ATOM | 342 | C | ASP | A | 22 | 46.054 | −2.652 | 23.717 | 1.00 | 68.07 | C |
| ATOM | 343 | O | ASP | A | 22 | 44.945 | −2.133 | 23.790 | 1.00 | 67.64 | O |
| ATOM | 344 | N | HIS | A | 23 | 46.807 | −2.553 | 22.638 | 1.00 | 65.04 | N |
| ATOM | 346 | CA | HIS | A | 23 | 46.409 | −1.772 | 21.532 | 1.00 | 61.33 | C |
| ATOM | 348 | CB | HIS | A | 23 | 47.586 | −1.723 | 20.576 | 1.00 | 60.16 | C |
| ATOM | 351 | CG | HIS | A | 23 | 47.430 | −0.802 | 19.398 | 1.00 | 57.41 | C |
| ATOM | 352 | ND1 | HIS | A | 23 | 47.673 | 0.552 | 19.463 | 1.00 | 58.45 | N |
| ATOM | 354 | CE1 | HIS | A | 23 | 47.519 | 1.098 | 18.275 | 1.00 | 53.82 | C |
| ATOM | 356 | NE2 | HIS | A | 23 | 47.197 | 0.141 | 17.433 | 1.00 | 55.61 | N |
| ATOM | 358 | CD2 | HIS | A | 23 | 47.171 | −1.062 | 18.100 | 1.00 | 54.92 | C |
| ATOM | 360 | C | HIS | A | 23 | 45.178 | −2.497 | 21.012 | 1.00 | 59.61 | C |
| ATOM | 361 | O | HIS | A | 23 | 44.118 | −1.917 | 20.865 | 1.00 | 59.40 | O |
| ATOM | 362 | N | PHE | A | 24 | 45.286 | −3.789 | 20.800 | 1.00 | 59.94 | N |
| ATOM | 364 | CA | PHE | A | 24 | 44.165 | −4.543 | 20.272 | 1.00 | 58.09 | C |
| ATOM | 366 | CB | PHE | A | 24 | 44.597 | −5.974 | 19.965 | 1.00 | 58.40 | C |
| ATOM | 369 | CG | PHE | A | 24 | 43.471 | −6.884 | 19.627 | 1.00 | 56.74 | C |
| ATOM | 370 | CD1 | PHE | A | 24 | 43.023 | −6.988 | 18.340 | 1.00 | 54.72 | C |
| ATOM | 372 | CE1 | PHE | A | 24 | 41.961 | −7.863 | 18.065 | 1.00 | 55.93 | C |
| ATOM | 374 | CZ | PHE | A | 24 | 41.358 | −8.614 | 19.113 | 1.00 | 55.91 | C |
| ATOM | 376 | CE2 | PHE | A | 24 | 41.803 | −8.486 | 20.339 | 1.00 | 56.90 | C |
| ATOM | 378 | CD2 | PHE | A | 24 | 42.844 | −7.634 | 20.609 | 1.00 | 56.82 | C |
| ATOM | 380 | C | PHE | A | 24 | 42.939 | −4.503 | 21.203 | 1.00 | 59.52 | C |
| ATOM | 381 | O | PHE | A | 24 | 41.843 | −4.307 | 20.754 | 1.00 | 58.02 | O |
| ATOM | 382 | N | LEU | A | 25 | 43.108 | −4.645 | 22.501 | 1.00 | 63.07 | N |
| ATOM | 384 | CA | LEU | A | 25 | 41.933 | −4.666 | 23.381 | 1.00 | 65.30 | C |
| ATOM | 386 | CB | LEU | A | 25 | 42.314 | −4.821 | 24.840 | 1.00 | 68.45 | C |
| ATOM | 389 | CG | LEU | A | 25 | 43.082 | −6.114 | 25.072 | 1.00 | 72.33 | C |
| ATOM | 391 | CD1 | LEU | A | 25 | 43.607 | −6.190 | 26.513 | 1.00 | 78.48 | C |
| ATOM | 395 | CD2 | LEU | A | 25 | 42.279 | −7.433 | 24.705 | 1.00 | 72.81 | C |
| ATOM | 399 | C | LEU | A | 25 | 41.117 | −3.401 | 23.190 | 1.00 | 64.01 | C |
| ATOM | 400 | O | LEU | A | 25 | 39.876 | −3.424 | 23.279 | 1.00 | 63.70 | O |
| ATOM | 401 | N | SER | A | 26 | 41.826 | −2.309 | 22.885 | 1.00 | 63.31 | N |
| ATOM | 403 | CA | SER | A | 26 | 41.237 | −0.975 | 22.871 | 1.00 | 63.14 | C |
| ATOM | 405 | CB | SER | A | 26 | 42.233 | 0.064 | 23.416 | 1.00 | 64.86 | C |
| ATOM | 408 | OG | SER | A | 26 | 42.684 | 0.916 | 22.406 | 1.00 | 65.68 | O |
| ATOM | 410 | C | SER | A | 26 | 40.693 | −0.634 | 21.502 | 1.00 | 59.23 | C |
| ATOM | 411 | O | SER | A | 26 | 39.666 | 0.067 | 21.392 | 1.00 | 58.89 | O |
| ATOM | 412 | N | LEU | A | 27 | 41.375 | −1.155 | 20.478 | 1.00 | 56.73 | N |
| ATOM | 414 | CA | LEU | A | 27 | 40.789 | −1.286 | 19.177 | 1.00 | 53.81 | C |
| ATOM | 416 | CB | LEU | A | 27 | 41.575 | −2.211 | 18.289 | 1.00 | 52.35 | C |
| ATOM | 419 | CG | LEU | A | 27 | 41.132 | −2.136 | 16.819 | 1.00 | 49.44 | C |
| ATOM | 421 | CD1 | LEU | A | 27 | 41.376 | −0.797 | 16.298 | 1.00 | 46.75 | C |
| ATOM | 425 | CD2 | LEU | A | 27 | 41.881 | −3.150 | 15.889 | 1.00 | 49.11 | C |
| ATOM | 429 | C | LEU | A | 27 | 39.461 | −1.911 | 19.358 | 1.00 | 54.56 | C |
| ATOM | 430 | O | LEU | A | 27 | 38.466 | −1.407 | 18.837 | 1.00 | 55.41 | O |
| ATOM | 431 | N | GLN | A | 28 | 39.456 | −3.009 | 20.096 | 1.00 | 55.62 | N |
| ATOM | 433 | CA | GLN | A | 28 | 38.281 | −3.800 | 20.297 | 1.00 | 56.39 | C |
| ATOM | 435 | CB | GLN | A | 28 | 38.618 | −5.091 | 21.063 | 1.00 | 59.09 | C |
| ATOM | 438 | CG | GLN | A | 28 | 37.773 | −6.291 | 20.641 | 1.00 | 60.00 | C |
| ATOM | 441 | CD | GLN | A | 28 | 38.007 | −7.567 | 21.488 | 1.00 | 64.33 | C |
| ATOM | 442 | OE1 | GLN | A | 28 | 38.723 | −7.563 | 22.480 | 1.00 | 64.48 | O |
| ATOM | 443 | NE2 | GLN | A | 28 | 37.405 | −8.649 | 21.059 | 1.00 | 65.54 | N |
| ATOM | 446 | C | GLN | A | 28 | 37.210 | −3.034 | 21.026 | 1.00 | 57.59 | C |
| ATOM | 447 | O | GLN | A | 28 | 36.029 | −3.157 | 20.656 | 1.00 | 57.19 | O |
| ATOM | 448 | N | ARG | A | 29 | 37.594 | −2.271 | 22.063 | 1.00 | 59.69 | N |
| ATOM | 450 | CA | ARG | A | 29 | 36.584 | −1.653 | 22.971 | 1.00 | 62.22 | C |
| ATOM | 452 | CB | ARG | A | 29 | 37.133 | −1.079 | 24.311 | 1.00 | 64.71 | C |
| ATOM | 461 | C | ARG | A | 29 | 35.845 | −0.627 | 22.145 | 1.00 | 60.09 | C |
| ATOM | 462 | O | ARG | A | 29 | 34.649 | −0.509 | 22.287 | 1.00 | 61.92 | O |
| ATOM | 463 | N | MET | A | 30 | 36.547 | 0.031 | 21.226 | 1.00 | 57.09 | N |
| ATOM | 465 | CA | MET | A | 30 | 35.975 | 1.057 | 20.390 | 1.00 | 55.26 | C |
| ATOM | 467 | CB | MET | A | 30 | 37.106 | 1.794 | 19.716 | 1.00 | 53.16 | C |
| ATOM | 470 | CG | MET | A | 30 | 36.722 | 2.870 | 18.699 | 1.00 | 51.84 | C |
| ATOM | 473 | SD | MET | A | 30 | 35.550 | 4.071 | 19.326 | 1.00 | 54.54 | S |
| ATOM | 474 | CE | MET | A | 30 | 36.573 | 5.055 | 20.420 | 1.00 | 55.60 | C |
| ATOM | 478 | C | MET | A | 30 | 35.059 | 0.480 | 19.332 | 1.00 | 53.92 | C |
| ATOM | 479 | O | MET | A | 30 | 33.907 | 0.863 | 19.200 | 1.00 | 55.74 | O |
| ATOM | 480 | N | PHE | A | 31 | 35.550 | −0.468 | 18.559 | 1.00 | 52.04 | N |
| ATOM | 482 | CA | PHE | A | 31 | 34.781 | −0.943 | 17.430 | 1.00 | 49.68 | C |
| ATOM | 484 | CB | PHE | A | 31 | 35.756 | −1.193 | 16.273 | 1.00 | 47.66 | C |
| ATOM | 487 | CG | PHE | A | 31 | 36.433 | 0.085 | 15.783 | 1.00 | 46.71 | C |
| ATOM | 488 | CD1 | PHE | A | 31 | 37.781 | 0.305 | 15.979 | 1.00 | 46.45 | C |
| ATOM | 490 | CE1 | PHE | A | 31 | 38.321 | 1.541 | 15.566 | 1.00 | 47.62 | C |
| ATOM | 492 | CZ | PHE | A | 31 | 37.509 | 2.502 | 14.959 | 1.00 | 44.30 | C |
| ATOM | 494 | CE2 | PHE | A | 31 | 36.194 | 2.278 | 14.788 | 1.00 | 42.28 | C |
| ATOM | 496 | CD2 | PHE | A | 31 | 35.663 | 1.104 | 15.190 | 1.00 | 44.72 | C |
| ATOM | 498 | C | PHE | A | 31 | 33.789 | −2.089 | 17.673 | 1.00 | 50.47 | C |
| ATOM | 499 | O | PHE | A | 31 | 33.081 | −2.417 | 16.777 | 1.00 | 48.38 | O |
| ATOM | 500 | N | ASN | A | 32 | 33.613 | −2.585 | 18.890 | 1.00 | 53.44 | N |
| ATOM | 502 | CA | ASN | A | 32 | 32.885 | −3.794 | 19.024 | 1.00 | 56.24 | C |
| ATOM | 504 | CB | ASN | A | 32 | 32.747 | −4.260 | 20.430 | 1.00 | 59.73 | C |
| ATOM | 507 | CG | ASN | A | 32 | 32.163 | −5.711 | 20.520 | 1.00 | 62.37 | C |
| ATOM | 508 | OD1 | ASN | A | 32 | 32.440 | −6.605 | 19.743 | 1.00 | 58.57 | O |
| ATOM | 509 | ND2 | ASN | A | 32 | 31.353 | −5.911 | 21.504 | 1.00 | 72.53 | N |
| ATOM | 512 | C | ASN | A | 32 | 31.558 | −3.958 | 18.295 | 1.00 | 58.05 | C |
| ATOM | 513 | O | ASN | A | 32 | 31.416 | −4.866 | 17.436 | 1.00 | 59.26 | O |
| ATOM | 514 | N | ASN | A | 33 | 30.525 | −3.217 | 18.586 | 1.00 | 60.46 | N |
| ATOM | 516 | CA | ASN | A | 33 | 29.286 | −3.562 | 17.838 | 1.00 | 61.41 | C |
| ATOM | 518 | CB | ASN | A | 33 | 28.089 | −3.573 | 18.789 | 1.00 | 65.21 | C |
| ATOM | 521 | CG | ASN | A | 33 | 28.127 | −4.776 | 19.664 | 1.00 | 67.90 | C |
| ATOM | 522 | OD1 | ASN | A | 33 | 28.491 | −5.890 | 19.232 | 1.00 | 63.09 | O |
| ATOM | 523 | ND2 | ASN | A | 33 | 27.793 | −4.578 | 20.898 | 1.00 | 73.36 | N |
| ATOM | 526 | C | ASN | A | 33 | 29.053 | −2.692 | 16.659 | 1.00 | 59.01 | C |
| ATOM | 527 | O | ASN | A | 33 | 27.930 | −2.561 | 16.195 | 1.00 | 60.95 | O |
| ATOM | 528 | N | CYS | A | 34 | 30.114 | −2.094 | 16.167 | 1.00 | 55.98 | N |
| ATOM | 530 | CA | CYS | A | 34 | 29.963 | −0.850 | 15.449 | 1.00 | 55.77 | C |
| ATOM | 532 | CB | CYS | A | 34 | 31.281 | −0.106 | 15.502 | 1.00 | 54.43 | C |
| ATOM | 535 | SG | CYS | A | 34 | 31.232 | 1.564 | 14.851 | 1.00 | 57.88 | S |
| ATOM | 536 | C | CYS | A | 34 | 29.476 | −0.995 | 13.995 | 1.00 | 54.12 | C |
| ATOM | 537 | O | CYS | A | 34 | 30.046 | −1.728 | 13.154 | 1.00 | 52.55 | O |
| ATOM | 538 | N | GLU | A | 35 | 28.408 | −0.279 | 13.701 | 1.00 | 54.92 | N |
| ATOM | 540 | CA | GLU | A | 35 | 27.854 | −0.268 | 12.353 | 1.00 | 54.01 | C |
| ATOM | 542 | CB | GLU | A | 35 | 26.296 | −0.439 | 12.423 | 1.00 | 57.63 | C |
| ATOM | 545 | CG | GLU | A | 35 | 25.807 | −1.904 | 12.595 | 1.00 | 60.04 | C |
| ATOM | 548 | CD | GLU | A | 35 | 24.265 | −2.089 | 12.755 | 1.00 | 66.48 | C |
| ATOM | 549 | OE1 | GLU | A | 35 | 23.430 | −1.283 | 12.291 | 1.00 | 69.81 | O |
| ATOM | 550 | OE2 | GLU | A | 35 | 23.846 | −3.086 | 13.360 | 1.00 | 69.46 | O |
| ATOM | 551 | C | GLU | A | 35 | 28.366 | 0.985 | 11.531 | 1.00 | 50.84 | C |
| ATOM | 552 | O | GLU | A | 35 | 28.835 | 0.861 | 10.394 | 1.00 | 47.39 | O |
| ATOM | 553 | N | VAL | A | 36 | 28.298 | 2.154 | 12.164 | 1.00 | 51.35 | N |
| ATOM | 555 | CA | VAL | A | 36 | 28.759 | 3.440 | 11.634 | 1.00 | 49.75 | C |
| ATOM | 557 | CB | VAL | A | 36 | 27.613 | 4.468 | 11.651 | 1.00 | 52.18 | C |
| ATOM | 559 | CG1 | VAL | A | 36 | 28.075 | 5.785 | 11.084 | 1.00 | 50.62 | C |
| ATOM | 563 | CG2 | VAL | A | 36 | 26.441 | 3.964 | 10.857 | 1.00 | 53.96 | C |
| ATOM | 567 | C | VAL | A | 36 | 29.898 | 4.062 | 12.454 | 1.00 | 48.86 | C |
| ATOM | 568 | O | VAL | A | 36 | 29.810 | 4.215 | 13.664 | 1.00 | 50.58 | O |
| ATOM | 569 | N | VAL | A | 37 | 30.955 | 4.440 | 11.786 | 1.00 | 46.36 | N |
| ATOM | 571 | CA | VAL | A | 37 | 32.030 | 5.169 | 12.417 | 1.00 | 46.84 | C |
| ATOM | 573 | CB | VAL | A | 37 | 33.361 | 4.637 | 11.878 | 1.00 | 44.64 | C |
| ATOM | 575 | CG1 | VAL | A | 37 | 34.537 | 5.214 | 12.638 | 1.00 | 45.69 | C |
| ATOM | 579 | CG2 | VAL | A | 37 | 33.372 | 3.171 | 11.950 | 1.00 | 43.09 | C |
| ATOM | 583 | C | VAL | A | 37 | 31.876 | 6.682 | 12.091 | 1.00 | 47.84 | C |
| ATOM | 584 | O | VAL | A | 37 | 32.128 | 7.150 | 10.959 | 1.00 | 47.48 | O |
| ATOM | 585 | N | LEU | A | 38 | 31.406 | 7.446 | 13.057 | 1.00 | 49.78 | N |
| ATOM | 587 | CA | LEU | A | 38 | 31.412 | 8.917 | 12.977 | 1.00 | 50.77 | C |
| ATOM | 589 | CB | LEU | A | 38 | 31.068 | 9.494 | 14.341 | 1.00 | 53.03 | C |
| ATOM | 592 | CG | LEU | A | 38 | 29.739 | 9.093 | 14.919 | 1.00 | 54.79 | C |
| ATOM | 594 | CD1 | LEU | A | 38 | 29.401 | 10.042 | 16.017 | 1.00 | 57.99 | C |
| ATOM | 598 | CD2 | LEU | A | 38 | 28.708 | 9.110 | 13.851 | 1.00 | 54.29 | C |
| ATOM | 602 | C | LEU | A | 38 | 32.752 | 9.540 | 12.550 | 1.00 | 49.42 | C |
| ATOM | 603 | O | LEU | A | 38 | 32.748 | 10.523 | 11.843 | 1.00 | 50.78 | O |
| ATOM | 604 | N | GLY | A | 39 | 33.862 | 8.991 | 13.049 | 1.00 | 48.45 | N |
| ATOM | 606 | CA | GLY | A | 39 | 35.219 | 9.454 | 12.840 | 1.00 | 47.35 | C |
| ATOM | 609 | C | GLY | A | 39 | 35.961 | 8.563 | 11.878 | 1.00 | 45.06 | C |
| ATOM | 610 | O | GLY | A | 39 | 35.409 | 8.141 | 10.867 | 1.00 | 45.58 | O |
| ATOM | 611 | N | ASN | A | 40 | 37.225 | 8.286 | 12.136 | 1.00 | 44.47 | N |
| ATOM | 613 | CA | ASN | A | 40 | 37.921 | 7.385 | 11.256 | 1.00 | 42.80 | C |
| ATOM | 615 | CB | ASN | A | 40 | 39.282 | 7.891 | 10.781 | 1.00 | 43.45 | C |
| ATOM | 618 | CG | ASN | A | 40 | 39.429 | 9.389 | 10.797 | 1.00 | 44.32 | C |
| ATOM | 619 | OD1 | ASN | A | 40 | 38.929 | 10.062 | 9.935 | 1.00 | 45.99 | O |
| ATOM | 620 | ND2 | ASN | A | 40 | 40.205 | 9.897 | 11.734 | 1.00 | 44.54 | N |
| ATOM | 623 | C | ASN | A | 40 | 38.159 | 6.049 | 11.918 | 1.00 | 42.57 | C |
| ATOM | 624 | O | ASN | A | 40 | 38.211 | 5.931 | 13.148 | 1.00 | 44.09 | O |
| ATOM | 625 | N | LEU | A | 41 | 38.390 | 5.082 | 11.046 | 1.00 | 40.76 | N |
| ATOM | 627 | CA | LEU | A | 41 | 38.610 | 3.727 | 11.369 | 1.00 | 40.75 | C |
| ATOM | 629 | CB | LEU | A | 41 | 37.822 | 2.850 | 10.400 | 1.00 | 39.36 | C |
| ATOM | 632 | CG | LEU | A | 41 | 38.090 | 1.346 | 10.561 | 1.00 | 40.49 | C |
| ATOM | 634 | CD1 | LEU | A | 41 | 37.763 | 0.779 | 11.990 | 1.00 | 44.00 | C |
| ATOM | 638 | CD2 | LEU | A | 41 | 37.247 | 0.567 | 9.646 | 1.00 | 41.86 | C |
| ATOM | 642 | C | LEU | A | 41 | 40.058 | 3.488 | 11.150 | 1.00 | 40.97 | C |
| ATOM | 643 | O | LEU | A | 41 | 40.525 | 3.672 | 10.045 | 1.00 | 41.21 | O |
| ATOM | 644 | N | GLU | A | 42 | 40.785 | 3.046 | 12.166 | 1.00 | 42.33 | N |
| ATOM | 646 | CA | GLU | A | 42 | 42.201 | 2.775 | 12.003 | 1.00 | 42.70 | C |
| ATOM | 648 | CB | GLU | A | 42 | 43.020 | 3.721 | 12.808 | 1.00 | 44.36 | C |
| ATOM | 651 | CG | GLU | A | 42 | 42.695 | 5.159 | 12.455 | 1.00 | 47.91 | C |
| ATOM | 654 | CD | GLU | A | 42 | 43.418 | 6.201 | 13.308 | 1.00 | 51.77 | C |
| ATOM | 655 | OE1 | GLU | A | 42 | 43.332 | 6.066 | 14.581 | 1.00 | 51.29 | O |
| ATOM | 656 | OE2 | GLU | A | 42 | 44.027 | 7.154 | 12.688 | 1.00 | 51.12 | O |
| ATOM | 657 | C | GLU | A | 42 | 42.486 | 1.426 | 12.488 | 1.00 | 43.28 | C |
| ATOM | 658 | O | GLU | A | 42 | 42.282 | 1.164 | 13.667 | 1.00 | 45.62 | O |
| ATOM | 659 | N | ILE | A | 43 | 42.979 | 0.557 | 11.605 | 1.00 | 42.12 | N |
| ATOM | 661 | CA | ILE | A | 43 | 43.421 | −0.746 | 12.046 | 1.00 | 42.21 | C |
| ATOM | 663 | CB | ILE | A | 43 | 42.633 | −1.771 | 11.341 | 1.00 | 40.74 | C |
| ATOM | 665 | CG1 | ILE | A | 43 | 41.166 | −1.548 | 11.668 | 1.00 | 39.80 | C |
| ATOM | 668 | CD1 | ILE | A | 43 | 40.301 | −1.940 | 10.641 | 1.00 | 39.39 | C |
| ATOM | 672 | CG2 | ILE | A | 43 | 43.052 | −3.107 | 11.840 | 1.00 | 43.58 | C |
| ATOM | 676 | C | ILE | A | 43 | 44.919 | −0.912 | 11.876 | 1.00 | 42.70 | C |
| ATOM | 677 | O | ILE | A | 43 | 45.477 | −0.694 | 10.858 | 1.00 | 42.81 | O |
| ATOM | 678 | N | THR | A | 44 | 45.555 | −1.355 | 12.916 | 1.00 | 44.98 | N |
| ATOM | 680 | CA | THR | A | 44 | 46.888 | −0.922 | 13.235 | 1.00 | 46.86 | C |
| ATOM | 682 | CB | THR | A | 44 | 46.703 | 0.473 | 13.748 | 1.00 | 47.31 | C |
| ATOM | 684 | OG1 | THR | A | 44 | 46.847 | 1.369 | 12.637 | 1.00 | 48.62 | O |
| ATOM | 686 | CG2 | THR | A | 44 | 47.744 | 0.877 | 14.643 | 1.00 | 49.36 | C |
| ATOM | 690 | C | THR | A | 44 | 47.609 | −1.798 | 14.277 | 1.00 | 49.13 | C |
| ATOM | 691 | O | THR | A | 44 | 47.102 | −2.055 | 15.380 | 1.00 | 49.25 | O |
| ATOM | 692 | N | TYR | A | 45 | 48.794 | −2.258 | 13.899 | 1.00 | 50.28 | N |
| ATOM | 694 | CA | TYR | A | 45 | 49.613 | −3.069 | 14.773 | 1.00 | 52.93 | C |
| ATOM | 696 | CB | TYR | A | 45 | 50.095 | −2.247 | 15.992 | 1.00 | 55.11 | C |
| ATOM | 699 | CG | TYR | A | 45 | 50.992 | −1.106 | 15.593 | 1.00 | 55.23 | C |
| ATOM | 700 | CD1 | TYR | A | 45 | 50.530 | 0.165 | 15.617 | 1.00 | 54.89 | C |
| ATOM | 702 | CE1 | TYR | A | 45 | 51.299 | 1.209 | 15.259 | 1.00 | 54.93 | C |
| ATOM | 704 | CZ | TYR | A | 45 | 52.553 | 0.999 | 14.808 | 1.00 | 56.32 | C |
| ATOM | 705 | OH | TYR | A | 45 | 53.273 | 2.109 | 14.459 | 1.00 | 62.09 | O |
| ATOM | 707 | CE2 | TYR | A | 45 | 53.066 | −0.250 | 14.754 | 1.00 | 56.10 | C |
| ATOM | 709 | CD2 | TYR | A | 45 | 52.279 | −1.306 | 15.143 | 1.00 | 56.61 | C |
| ATOM | 711 | C | TYR | A | 45 | 48.946 | −4.393 | 15.189 | 1.00 | 53.13 | C |
| ATOM | 712 | O | TYR | A | 45 | 49.477 | −5.102 | 16.030 | 1.00 | 56.05 | O |
| ATOM | 713 | N | VAL | A | 46 | 47.843 | −4.775 | 14.559 | 1.00 | 50.80 | N |
| ATOM | 715 | CA | VAL | A | 46 | 47.215 | −6.039 | 14.908 | 1.00 | 51.76 | C |
| ATOM | 717 | CB | VAL | A | 46 | 45.861 | −6.206 | 14.238 | 1.00 | 49.39 | C |
| ATOM | 719 | CG1 | VAL | A | 46 | 45.204 | −7.480 | 14.608 | 1.00 | 50.02 | C |
| ATOM | 723 | CG2 | VAL | A | 46 | 44.963 | −5.087 | 14.699 | 1.00 | 51.23 | C |
| ATOM | 727 | C | VAL | A | 46 | 48.118 | −7.153 | 14.509 | 1.00 | 53.22 | C |
| ATOM | 728 | O | VAL | A | 46 | 48.496 | −7.203 | 13.361 | 1.00 | 53.23 | O |
| ATOM | 729 | N | GLN | A | 47 | 48.430 | −8.050 | 15.436 | 1.00 | 56.14 | N |
| ATOM | 731 | CA | GLN | A | 47 | 49.344 | −9.151 | 15.162 | 1.00 | 59.49 | C |
| ATOM | 733 | CB | GLN | A | 47 | 50.283 | −9.340 | 16.373 | 1.00 | 63.39 | C |
| ATOM | 736 | CG | GLN | A | 47 | 51.212 | −8.130 | 16.701 | 1.00 | 64.49 | C |
| ATOM | 739 | CD | GLN | A | 47 | 52.252 | −7.834 | 15.592 | 1.00 | 64.62 | C |
| ATOM | 740 | OE1 | GLN | A | 47 | 52.947 | −8.741 | 15.096 | 1.00 | 64.30 | O |
| ATOM | 741 | NE2 | GLN | A | 47 | 52.335 | −6.564 | 15.191 | 1.00 | 65.38 | N |
| ATOM | 744 | C | GLN | A | 47 | 48.684 | −10.517 | 14.730 | 1.00 | 60.25 | C |
| ATOM | 745 | O | GLN | A | 47 | 47.461 | −10.670 | 14.710 | 1.00 | 59.39 | O |
| ATOM | 746 | N | ARG | A | 48 | 49.500 | −11.502 | 14.381 | 1.00 | 62.64 | N |
| ATOM | 748 | CA | ARG | A | 48 | 48.950 | −12.787 | 13.940 | 1.00 | 64.49 | C |
| ATOM | 750 | CB | ARG | A | 48 | 50.048 | −13.799 | 13.534 | 1.00 | 67.77 | C |
| ATOM | 753 | CG | ARG | A | 48 | 50.278 | −13.822 | 12.038 | 1.00 | 68.04 | C |
| ATOM | 756 | CD | ARG | A | 48 | 51.286 | −14.816 | 11.673 | 1.00 | 74.89 | C |
| ATOM | 759 | NE | ARG | A | 48 | 50.712 | −15.969 | 10.921 | 1.00 | 78.86 | N |
| ATOM | 761 | CZ | ARG | A | 48 | 50.734 | −17.271 | 11.322 | 1.00 | 76.74 | C |
| ATOM | 762 | NH1 | ARG | A | 48 | 51.286 | −17.691 | 12.474 | 1.00 | 78.50 | N |
| ATOM | 765 | NH2 | ARG | A | 48 | 50.196 | −18.159 | 10.526 | 1.00 | 78.65 | N |
| ATOM | 768 | C | ARG | A | 48 | 48.077 | −13.463 | 14.977 | 1.00 | 65.82 | C |
| ATOM | 769 | O | ARG | A | 48 | 48.387 | −13.433 | 16.148 | 1.00 | 69.06 | O |
| ATOM | 770 | N | ASN | A | 49 | 47.020 | −14.120 | 14.508 | 1.00 | 64.58 | N |
| ATOM | 772 | CA | ASN | A | 49 | 46.070 | −14.867 | 15.340 | 1.00 | 65.41 | C |
| ATOM | 774 | CB | ASN | A | 49 | 46.724 | −16.066 | 16.015 | 1.00 | 69.43 | C |
| ATOM | 777 | CG | ASN | A | 49 | 47.310 | −16.988 | 15.043 | 1.00 | 70.04 | C |
| ATOM | 778 | OD1 | ASN | A | 49 | 46.635 | −17.426 | 14.102 | 1.00 | 68.58 | O |
| ATOM | 779 | ND2 | ASN | A | 49 | 48.587 | −17.271 | 15.207 | 1.00 | 73.31 | N |
| ATOM | 782 | C | ASN | A | 49 | 45.334 | −14.079 | 16.368 | 1.00 | 64.69 | C |
| ATOM | 783 | O | ASN | A | 49 | 44.878 | −14.646 | 17.355 | 1.00 | 66.94 | O |
| ATOM | 784 | N | TYR | A | 50 | 45.214 | −12.785 | 16.166 | 1.00 | 61.48 | N |
| ATOM | 786 | CA | TYR | A | 50 | 44.278 | −12.059 | 16.973 | 1.00 | 62.03 | C |
| ATOM | 788 | CB | TYR | A | 50 | 44.795 | −10.662 | 17.261 | 1.00 | 61.43 | C |
| ATOM | 791 | CG | TYR | A | 50 | 45.768 | −10.556 | 18.432 | 1.00 | 64.24 | C |
| ATOM | 792 | CD1 | TYR | A | 50 | 47.030 | −11.118 | 18.363 | 1.00 | 65.32 | C |
| ATOM | 794 | CE1 | TYR | A | 50 | 47.922 | −10.989 | 19.436 | 1.00 | 69.50 | C |
| ATOM | 796 | CZ | TYR | A | 50 | 47.553 | −10.279 | 20.561 | 1.00 | 70.19 | C |
| ATOM | 797 | OH | TYR | A | 50 | 48.425 | −10.156 | 21.594 | 1.00 | 74.47 | O |
| ATOM | 799 | CE2 | TYR | A | 50 | 46.328 | −9.705 | 20.642 | 1.00 | 67.54 | C |
| ATOM | 801 | CD2 | TYR | A | 50 | 45.446 | −9.818 | 19.573 | 1.00 | 64.01 | C |
| ATOM | 803 | C | TYR | A | 50 | 42.992 | −11.996 | 16.156 | 1.00 | 59.86 | C |
| ATOM | 804 | O | TYR | A | 50 | 43.082 | −11.703 | 15.011 | 1.00 | 57.85 | O |
| ATOM | 805 | N | ASP | A | 51 | 41.818 | −12.223 | 16.752 | 1.00 | 61.02 | N |
| ATOM | 807 | CA | ASP | A | 51 | 40.540 | −12.225 | 16.037 | 1.00 | 59.60 | C |
| ATOM | 809 | CB | ASP | A | 51 | 39.472 | −12.905 | 16.896 | 1.00 | 62.57 | C |
| ATOM | 812 | CG | ASP | A | 51 | 38.272 | −13.445 | 16.079 | 1.00 | 61.90 | C |
| ATOM | 813 | OD1 | ASP | A | 51 | 38.043 | −13.027 | 14.914 | 1.00 | 53.66 | O |
| ATOM | 814 | OD2 | ASP | A | 51 | 37.498 | −14.330 | 16.570 | 1.00 | 65.06 | O |
| ATOM | 815 | C | ASP | A | 51 | 40.013 | −10.838 | 15.775 | 1.00 | 57.33 | C |
| ATOM | 816 | O | ASP | A | 51 | 39.564 | −10.219 | 16.717 | 1.00 | 59.76 | O |
| ATOM | 817 | N | LEU | A | 52 | 39.985 | −10.397 | 14.519 | 1.00 | 54.26 | N |
| ATOM | 819 | CA | LEU | A | 52 | 39.331 | −9.145 | 14.121 | 1.00 | 52.34 | C |
| ATOM | 821 | CB | LEU | A | 52 | 40.110 | −8.415 | 13.029 | 1.00 | 49.67 | C |
| ATOM | 824 | CG | LEU | A | 52 | 41.456 | −7.893 | 13.423 | 1.00 | 50.34 | C |
| ATOM | 826 | CD1 | LEU | A | 52 | 42.092 | −7.459 | 12.115 | 1.00 | 49.85 | C |
| ATOM | 830 | CD2 | LEU | A | 52 | 41.305 | −6.728 | 14.427 | 1.00 | 50.52 | C |
| ATOM | 834 | C | LEU | A | 52 | 37.948 | −9.321 | 13.542 | 1.00 | 51.96 | C |
| ATOM | 835 | O | LEU | A | 52 | 37.444 | −8.450 | 12.848 | 1.00 | 50.66 | O |
| ATOM | 836 | N | SER | A | 53 | 37.293 | −10.399 | 13.864 | 1.00 | 54.16 | N |
| ATOM | 838 | CA | SER | A | 53 | 36.003 | −10.618 | 13.274 | 1.00 | 54.53 | C |
| ATOM | 840 | CB | SER | A | 53 | 35.526 | −12.042 | 13.555 | 1.00 | 56.90 | C |
| ATOM | 843 | OG | SER | A | 53 | 35.460 | −12.203 | 14.922 | 1.00 | 58.05 | O |
| ATOM | 845 | C | SER | A | 53 | 34.947 | −9.619 | 13.758 | 1.00 | 55.01 | C |
| ATOM | 846 | O | SER | A | 53 | 33.897 | −9.595 | 13.168 | 1.00 | 56.80 | O |
| ATOM | 847 | N | PHE | A | 54 | 35.168 | −8.820 | 14.802 | 1.00 | 54.98 | N |
| ATOM | 849 | CA | PHE | A | 54 | 34.142 | −7.845 | 15.190 | 1.00 | 55.18 | C |
| ATOM | 851 | CB | PHE | A | 54 | 34.467 | −7.156 | 16.478 | 1.00 | 55.26 | C |
| ATOM | 854 | CG | PHE | A | 54 | 35.840 | −6.619 | 16.535 | 1.00 | 53.23 | C |
| ATOM | 855 | CD1 | PHE | A | 54 | 36.104 | −5.318 | 16.141 | 1.00 | 55.25 | C |
| ATOM | 857 | CE1 | PHE | A | 54 | 37.420 | −4.754 | 16.207 | 1.00 | 53.92 | C |
| ATOM | 859 | CZ | PHE | A | 54 | 38.446 | −5.524 | 16.671 | 1.00 | 56.11 | C |
| ATOM | 861 | CE2 | PHE | A | 54 | 38.166 | −6.881 | 17.085 | 1.00 | 54.50 | C |
| ATOM | 863 | CD2 | PHE | A | 54 | 36.883 | −7.395 | 16.974 | 1.00 | 53.07 | C |
| ATOM | 865 | C | PHE | A | 54 | 33.936 | −6.763 | 14.167 | 1.00 | 54.02 | C |
| ATOM | 866 | O | PHE | A | 54 | 32.946 | −6.016 | 14.274 | 1.00 | 56.54 | O |
| ATOM | 867 | N | LEU | A | 55 | 34.895 | −6.616 | 13.233 | 1.00 | 51.66 | N |
| ATOM | 869 | CA | LEU | A | 55 | 34.779 | −5.672 | 12.103 | 1.00 | 48.92 | C |
| ATOM | 871 | CB | LEU | A | 55 | 36.115 | −5.550 | 11.426 | 1.00 | 46.55 | C |
| ATOM | 874 | CG | LEU | A | 55 | 37.205 | −5.064 | 12.318 | 1.00 | 46.74 | C |
| ATOM | 876 | CD1 | LEU | A | 55 | 38.490 | −5.184 | 11.621 | 1.00 | 45.91 | C |
| ATOM | 880 | CD2 | LEU | A | 55 | 36.9.62 | −3.616 | 12.600 | 1.00 | 47.82 | C |
| ATOM | 884 | C | LEU | A | 55 | 33.821 | −6.095 | 10.989 | 1.00 | 48.78 | C |
| ATOM | 885 | O | LEU | A | 55 | 33.656 | −5.390 | 9.989 | 1.00 | 49.08 | O |
| ATOM | 886 | N | LYS | A | 56 | 33.230 | −7.274 | 11.090 | 1.00 | 50.51 | N |
| ATOM | 888 | CA | LYS | A | 56 | 32.248 | −7.667 | 10.096 | 1.00 | 50.35 | C |
| ATOM | 890 | CB | LYS | A | 56 | 31.836 | −9.114 | 10.238 | 1.00 | 52.48 | C |
| ATOM | 893 | CG | LYS | A | 56 | 32.973 | −10.132 | 10.110 | 1.00 | 53.73 | C |
| ATOM | 896 | CD | LYS | A | 56 | 32.484 | −11.607 | 9.798 | 1.00 | 58.55 | C |
| ATOM | 899 | CE | LYS | A | 56 | 33.628 | −12.680 | 9.390 | 1.00 | 59.21 | C |
| ATOM | 902 | NZ | LYS | A | 56 | 33.071 | −13.963 | 8.832 | 1.00 | 61.23 | N |
| ATOM | 906 | C | LYS | A | 56 | 31.048 | −6.741 | 10.221 | 1.00 | 50.84 | C |
| ATOM | 907 | O | LYS | A | 56 | 30.296 | −6.625 | 9.287 | 1.00 | 51.82 | O |
| ATOM | 908 | N | THR | A | 57 | 30.905 | −6.036 | 11.339 | 1.00 | 51.04 | N |
| ATOM | 910 | CA | THR | A | 57 | 29.736 | −5.205 | 11.583 | 1.00 | 52.25 | C |
| ATOM | 912 | CB | THR | A | 57 | 29.699 | −4.985 | 13.031 | 1.00 | 53.38 | C |
| ATOM | 914 | OG1 | THR | A | 57 | 29.273 | −6.173 | 13.594 | 1.00 | 54.50 | O |
| ATOM | 916 | CG2 | THR | A | 57 | 28.567 | −4.211 | 13.417 | 1.00 | 58.64 | C |
| ATOM | 920 | C | THR | A | 57 | 29.757 | −3.823 | 10.876 | 1.00 | 50.73 | C |
| ATOM | 921 | O | THR | A | 57 | 28.726 | −3.142 | 10.690 | 1.00 | 50.93 | O |
| ATOM | 922 | N | ILE | A | 58 | 30.947 | −3.412 | 10.510 | 1.00 | 48.03 | N |
| ATOM | 924 | CA | ILE | A | 58 | 31.102 | −2.105 | 10.062 | 1.00 | 47.21 | C |
| ATOM | 926 | CB | ILE | A | 58 | 32.545 | −1.755 | 10.092 | 1.00 | 45.62 | C |
| ATOM | 928 | CG1 | ILE | A | 58 | 32.947 | −1.611 | 11.543 | 1.00 | 47.30 | C |
| ATOM | 931 | CD1 | ILE | A | 58 | 34.411 | −1.176 | 11.687 | 1.00 | 48.07 | C |
| ATOM | 935 | CG2 | ILE | A | 58 | 32.779 | −0.361 | 9.424 | 1.00 | 45.43 | C |
| ATOM | 939 | C | ILE | A | 58 | 30.472 | −1.946 | 8.683 | 1.00 | 46.76 | C |
| ATOM | 940 | O | ILE | A | 58 | 30.884 | −2.571 | 7.729 | 1.00 | 46.94 | O |
| ATOM | 941 | N | GLN | A | 59 | 29.457 | −1.091 | 8.633 | 1.00 | 47.32 | N |
| ATOM | 943 | CA | GLN | A | 59 | 28.765 | −0.744 | 7.433 | 1.00 | 46.32 | C |
| ATOM | 945 | CB | GLN | A | 59 | 27.308 | −0.535 | 7.789 | 1.00 | 49.40 | C |
| ATOM | 948 | CG | GLN | A | 59 | 26.460 | −1.801 | 7.748 | 1.00 | 50.88 | C |
| ATOM | 951 | CD | GLN | A | 59 | 25.154 | −1.630 | 8.490 | 1.00 | 56.62 | C |
| ATOM | 952 | OE1 | GLN | A | 59 | 24.282 | −0.851 | 8.084 | 1.00 | 61.17 | O |
| ATOM | 953 | NE2 | GLN | A | 59 | 25.029 | −2.319 | 9.621 | 1.00 | 62.39 | N |
| ATOM | 956 | C | GLN | A | 59 | 29.328 | 0.491 | 6.793 | 1.00 | 43.74 | C |
| ATOM | 957 | O | GLN | A | 59 | 29.321 | 0.621 | 5.609 | 1.00 | 42.04 | O |
| ATOM | 958 | N | GLU | A | 60 | 29.790 | 1.437 | 7.575 | 1.00 | 43.80 | N |
| ATOM | 960 | CA | GLU | A | 60 | 30.199 | 2.713 | 6.964 | 1.00 | 43.43 | C |
| ATOM | 962 | CB | GLU | A | 60 | 28.983 | 3.525 | 6.485 | 1.00 | 45.57 | C |
| ATOM | 965 | CG | GLU | A | 60 | 27.904 | 3.828 | 7.486 | 1.00 | 49.95 | C |
| ATOM | 968 | CD | GLU | A | 60 | 26.781 | 4.755 | 6.943 | 1.00 | 55.76 | C |
| ATOM | 969 | OE1 | GLU | A | 60 | 25.747 | 4.155 | 6.484 | 1.00 | 53.00 | O |
| ATOM | 970 | OE2 | GLU | A | 60 | 26.933 | 6.058 | 7.002 | 1.00 | 55.62 | O |
| ATOM | 971 | C | GLU | A | 60 | 31.110 | 3.560 | 7.813 | 1.00 | 41.36 | C |
| ATOM | 972 | O | GLU | A | 60 | 31.060 | 3.489 | 8.959 | 1.00 | 41.52 | O |
| ATOM | 973 | N | VAL | A | 61 | 31.956 | 4.343 | 7.171 | 1.00 | 40.34 | N |
| ATOM | 975 | CA | VAL | A | 61 | 32.955 | 5.212 | 7.787 | 1.00 | 40.06 | C |
| ATOM | 977 | CB | VAL | A | 61 | 34.383 | 4.620 | 7.589 | 1.00 | 38.27 | C |
| ATOM | 979 | CG1 | VAL | A | 61 | 35.479 | 5.597 | 8.136 | 1.00 | 38.94 | C |
| ATOM | 983 | CG2 | VAL | A | 61 | 34.453 | 3.245 | 8.246 | 1.00 | 36.00 | C |
| ATOM | 987 | C | VAL | A | 61 | 32.892 | 6.673 | 7.263 | 1.00 | 41.13 | C |
| ATOM | 988 | O | VAL | A | 61 | 33.035 | 7.009 | 6.073 | 1.00 | 40.36 | O |
| ATOM | 989 | N | ALA | A | 62 | 32.717 | 7.588 | 8.167 | 1.00 | 43.43 | N |
| ATOM | 991 | CA | ALA | A | 62 | 32.644 | 8.993 | 7.741 | 1.00 | 44.23 | C |
| ATOM | 993 | CB | ALA | A | 62 | 32.068 | 9.827 | 8.844 | 1.00 | 45.33 | C |
| ATOM | 997 | C | ALA | A | 62 | 34.000 | 9.548 | 7.301 | 1.00 | 42.41 | C |
| ATOM | 998 | O | ALA | A | 62 | 34.058 | 10.357 | 6.397 | 1.00 | 43.72 | O |
| ATOM | 999 | N | GLY | A | 63 | 35.062 | 9.160 | 7.984 | 1.00 | 41.42 | N |
| ATOM | 1001 | CA | GLY | A | 63 | 36.337 | 9.844 | 7.875 | 1.00 | 41.84 | C |
| ATOM | 1004 | C | GLY | A | 63 | 37.229 | 9.070 | 6.951 | 1.00 | 40.75 | C |
| ATOM | 1005 | O | GLY | A | 63 | 36.820 | 8.695 | 5.882 | 1.00 | 40.21 | O |
| ATOM | 1006 | N | TYR | A | 64 | 38.425 | 8.717 | 7.359 | 1.00 | 40.83 | N |
| ATOM | 1008 | CA | TYR | A | 64 | 39.185 | 7.771 | 6.561 | 1.00 | 39.12 | C |
| ATOM | 1010 | CB | TYR | A | 64 | 40.559 | 8.330 | 6.344 | 1.00 | 40.77 | C |
| ATOM | 1013 | CG | TYR | A | 64 | 41.366 | 8.605 | 7.595 | 1.00 | 40.87 | C |
| ATOM | 1014 | CD1 | TYR | A | 64 | 42.182 | 7.617 | 8.184 | 1.00 | 42.70 | C |
| ATOM | 1016 | CE1 | TYR | A | 64 | 42.969 | 7.892 | 9.305 | 1.00 | 44.16 | C |
| ATOM | 1018 | CZ | TYR | A | 64 | 42.910 | 9.171 | 9.833 | 1.00 | 42.92 | C |
| ATOM | 1019 | OH | TYR | A | 64 | 43.627 | 9.512 | 10.935 | 1.00 | 43.56 | O |
| ATOM | 1021 | CE2 | TYR | A | 64 | 42.090 | 10.108 | 9.279 | 1.00 | 43.84 | C |
| ATOM | 1023 | CD2 | TYR | A | 64 | 41.353 | 9.838 | 8.153 | 1.00 | 43.12 | C |
| ATOM | 1025 | C | TYR | A | 64 | 39.325 | 6.394 | 7.210 | 1.00 | 39.09 | C |
| ATOM | 1026 | O | TYR | A | 64 | 39.007 | 6.183 | 8.369 | 1.00 | 40.13 | O |
| ATOM | 1027 | N | VAL | A | 65 | 39.833 | 5.469 | 6.425 | 1.00 | 37.79 | N |
| ATOM | 1029 | CA | VAL | A | 65 | 40.165 | 4.157 | 6.845 | 1.00 | 37.58 | C |
| ATOM | 1031 | CB | VAL | A | 65 | 39.451 | 3.155 | 5.959 | 1.00 | 36.44 | C |
| ATOM | 1033 | CG1 | VAL | A | 65 | 39.941 | 1.695 | 6.176 | 1.00 | 35.49 | C |
| ATOM | 1037 | CG2 | VAL | A | 65 | 38.020 | 3.234 | 6.276 | 1.00 | 38.10 | C |
| ATOM | 1041 | C | VAL | A | 65 | 41.665 | 4.017 | 6.681 | 1.00 | 38.37 | C |
| ATOM | 1042 | O | VAL | A | 65 | 42.169 | 4.112 | 5.579 | 1.00 | 38.62 | O |
| ATOM | 1043 | N | LEU | A | 66 | 42.377 | 3.760 | 7.760 | 1.00 | 38.89 | N |
| ATOM | 1045 | CA | LEU | A | 66 | 43.750 | 3.428 | 7.621 | 1.00 | 40.32 | C |
| ATOM | 1047 | CB | LEU | A | 66 | 44.519 | 4.432 | 8.405 | 1.00 | 42.88 | C |
| ATOM | 1050 | CG | LEU | A | 66 | 45.904 | 4.043 | 8.875 | 1.00 | 45.09 | C |
| ATOM | 1052 | CD1 | LEU | A | 66 | 46.821 | 3.880 | 7.728 | 1.00 | 44.69 | C |
| ATOM | 1056 | CD2 | LEU | A | 66 | 46.373 | 5.153 | 9.836 | 1.00 | 48.02 | C |
| ATOM | 1060 | C | LEU | A | 66 | 44.042 | 2.058 | 8.155 | 1.00 | 40.80 | C |
| ATOM | 1061 | O | LEU | A | 66 | 43.832 | 1.757 | 9.314 | 1.00 | 42.77 | O |
| ATOM | 1062 | N | ILE | A | 67 | 44.576 | 1.198 | 7.326 | 1.00 | 40.95 | N |
| ATOM | 1064 | CA | ILE | A | 67 | 45.001 | −0.132 | 7.747 | 1.00 | 40.61 | C |
| ATOM | 1066 | CB | ILE | A | 67 | 44.324 | −1.113 | 6.861 | 1.00 | 39.42 | C |
| ATOM | 1068 | CG1 | ILE | A | 67 | 42.836 | −1.076 | 7.096 | 1.00 | 37.05 | C |
| ATOM | 1071 | CD1 | ILE | A | 67 | 42.129 | −1.575 | 5.966 | 1.00 | 36.61 | C |
| ATOM | 1075 | CG2 | ILE | A | 67 | 44.845 | −2.486 | 7.132 | 1.00 | 42.01 | C |
| ATOM | 1079 | C | ILE | A | 67 | 46.526 | −0.226 | 7.596 | 1.00 | 42.09 | C |
| ATOM | 1080 | O | ILE | A | 67 | 47.035 | −0.356 | 6.481 | 1.00 | 42.06 | O |
| ATOM | 1081 | N | ALA | A | 68 | 47.252 | −0.097 | 8.693 | 1.00 | 43.71 | N |
| ATOM | 1083 | CA | ALA | A | 68 | 48.691 | −0.174 | 8.634 | 1.00 | 46.04 | C |
| ATOM | 1085 | CB | ALA | A | 68 | 49.231 | 1.175 | 8.518 | 1.00 | 46.74 | C |
| ATOM | 1089 | C | ALA | A | 68 | 49.421 | −0.962 | 9.771 | 1.00 | 48.21 | C |
| ATOM | 1090 | O | ALA | A | 68 | 48.939 | −1.126 | 10.901 | 1.00 | 47.90 | O |
| ATOM | 1091 | N | LEU | A | 69 | 50.597 | −1.464 | 9.396 | 1.00 | 50.08 | N |
| ATOM | 1093 | CA | LEU | A | 69 | 51.542 | −2.143 | 10.292 | 1.00 | 52.36 | C |
| ATOM | 1095 | CB | LEU | A | 69 | 52.199 | −1.127 | 11.234 | 1.00 | 53.98 | C |
| ATOM | 1098 | CG | LEU | A | 69 | 52.815 | 0.105 | 10.537 | 1.00 | 54.54 | C |
| ATOM | 1100 | CD1 | LEU | A | 69 | 53.048 | 1.269 | 11.481 | 1.00 | 55.84 | C |
| ATOM | 1104 | CD2 | LEU | A | 69 | 54.114 | −0.210 | 9.926 | 1.00 | 56.72 | C |
| ATOM | 1108 | C | LEU | A | 69 | 50.917 | −3.355 | 10.993 | 1.00 | 51.43 | C |
| ATOM | 1109 | O | LEU | A | 69 | 51.253 | −3.718 | 12.102 | 1.00 | 52.82 | O |
| ATOM | 1110 | N | ASN | A | 70 | 50.034 | −3.989 | 10.254 | 1.00 | 49.53 | N |
| ATOM | 1112 | CA | ASN | A | 70 | 49.288 | −5.146 | 10.709 | 1.00 | 49.78 | C |
| ATOM | 1114 | CB | ASN | A | 70 | 47.887 | −5.148 | 10.071 | 1.00 | 47.45 | C |
| ATOM | 1117 | CG | ASN | A | 70 | 47.025 | −4.054 | 10.557 | 1.00 | 45.01 | C |
| ATOM | 1118 | OD1 | ASN | A | 70 | 46.654 | −4.043 | 11.719 | 1.00 | 45.13 | O |
| ATOM | 1119 | ND2 | ASN | A | 70 | 46.719 | −3.102 | 9.690 | 1.00 | 43.78 | N |
| ATOM | 1122 | C | ASN | A | 70 | 49.990 | −6.380 | 10.192 | 1.00 | 51.18 | C |
| ATOM | 1123 | O | ASN | A | 70 | 50.449 | −6.375 | 9.044 | 1.00 | 50.13 | O |
| ATOM | 1124 | N | THR | A | 71 | 50.106 | −7.425 | 11.001 | 1.00 | 53.00 | N |
| ATOM | 1126 | CA | THR | A | 71 | 50.513 | −8.709 | 10.427 | 1.00 | 55.45 | C |
| ATOM | 1128 | CB | THR | A | 71 | 51.718 | −9.356 | 11.116 | 1.00 | 58.70 | C |
| ATOM | 1130 | OG1 | THR | A | 71 | 51.432 | −9.593 | 12.492 | 1.00 | 59.65 | O |
| ATOM | 1132 | CG2 | THR | A | 71 | 52.909 | −8.463 | 11.068 | 1.00 | 60.02 | C |
| ATOM | 1136 | C | THR | A | 71 | 49.382 | −9.714 | 10.440 | 1.00 | 55.26 | C |
| ATOM | 1137 | O | THR | A | 71 | 49.550 | −10.818 | 9.952 | 1.00 | 55.74 | O |
| ATOM | 1138 | N | VAL | A | 72 | 48.242 | −9.316 | 11.005 | 1.00 | 53.94 | N |
| ATOM | 1140 | CA | VAL | A | 72 | 47.114 | −10.219 | 11.120 | 1.00 | 53.04 | C |
| ATOM | 1142 | CB | VAL | A | 72 | 45.909 | −9.515 | 11.839 | 1.00 | 51.69 | C |
| ATOM | 1144 | CG1 | VAL | A | 72 | 45.385 | −8.312 | 11.059 | 1.00 | 47.61 | C |
| ATOM | 1148 | CG2 | VAL | A | 72 | 44.786 | −10.530 | 12.214 | 1.00 | 51.56 | C |
| ATOM | 1152 | C | VAL | A | 72 | 46.800 | −10.776 | 9.727 | 1.00 | 51.50 | C |
| ATOM | 1153 | O | VAL | A | 72 | 46.932 | −10.096 | 8.734 | 1.00 | 50.25 | O |
| ATOM | 1154 | N | GLU | A | 73 | 46.456 | −12.047 | 9.672 | 1.00 | 53.42 | N |
| ATOM | 1156 | CA | GLU | A | 73 | 46.307 | −12.720 | 8.413 | 1.00 | 53.56 | C |
| ATOM | 1158 | CB | GLU | A | 73 | 46.091 | −14.219 | 8.575 | 1.00 | 55.45 | C |
| ATOM | 1161 | CG | GLU | A | 73 | 46.005 | −14.829 | 7.190 | 1.00 | 57.21 | C |
| ATOM | 1164 | CD | GLU | A | 73 | 46.159 | −16.328 | 7.121 | 1.00 | 65.02 | C |
| ATOM | 1165 | OE1 | GLU | A | 73 | 46.539 | −16.914 | 8.248 | 1.00 | 68.29 | O |
| ATOM | 1166 | OE2 | GLU | A | 73 | 45.894 | −16.869 | 5.933 | 1.00 | 62.47 | O |
| ATOM | 1167 | C | GLU | A | 73 | 45.169 | −12.057 | 7.647 | 1.00 | 51.07 | C |
| ATOM | 1168 | O | GLU | A | 73 | 45.351 | −11.655 | 6.539 | 1.00 | 49.97 | O |
| ATOM | 1169 | N | ARG | A | 74 | 44.011 | −11.888 | 8.257 | 1.00 | 50.85 | N |
| ATOM | 1171 | CA | ARG | A | 74 | 42.877 | −11.303 | 7.545 | 1.00 | 49.00 | C |
| ATOM | 1173 | CB | ARG | A | 74 | 41.874 | −12.437 | 7.294 | 1.00 | 49.93 | C |
| ATOM | 1176 | CG | ARG | A | 74 | 40.411 | −12.118 | 7.396 | 1.00 | 51.98 | C |
| ATOM | 1179 | CD | ARG | A | 74 | 39.468 | −13.326 | 7.038 | 1.00 | 54.93 | C |
| ATOM | 1182 | NE | ARG | A | 74 | 39.084 | −13.105 | 5.645 | 1.00 | 59.31 | N |
| ATOM | 1184 | CZ | ARG | A | 74 | 38.269 | −13.895 | 4.959 | 1.00 | 62.52 | C |
| ATOM | 1185 | NH1 | ARG | A | 74 | 37.762 | −14.941 | 5.616 | 1.00 | 65.45 | N |
| ATOM | 1188 | NH2 | ARG | A | 74 | 37.967 | −13.648 | 3.635 | 1.00 | 58.43 | N |
| ATOM | 1191 | C | ARG | A | 74 | 42.266 | −10.058 | 8.264 | 1.00 | 46.63 | C |
| ATOM | 1192 | O | ARG | A | 74 | 42.386 | −9.919 | 9.436 | 1.00 | 46.29 | O |
| ATOM | 1193 | N | ILE | A | 75 | 41.684 | −9.133 | 7.517 | 1.00 | 44.98 | N |
| ATOM | 1195 | CA | ILE | A | 75 | 40.955 | −7.991 | 8.086 | 1.00 | 43.65 | C |
| ATOM | 1197 | CB | ILE | A | 75 | 41.725 | −6.736 | 7.884 | 1.00 | 42.04 | C |
| ATOM | 1199 | CG1 | ILE | A | 75 | 42.831 | −6.684 | 8.929 | 1.00 | 43.21 | C |
| ATOM | 1202 | CD1 | ILE | A | 75 | 44.040 | −5.756 | 8.633 | 1.00 | 42.89 | C |
| ATOM | 1206 | CG2 | ILE | A | 75 | 40.805 | −5.538 | 8.084 | 1.00 | 42.87 | C |
| ATOM | 1210 | C | ILE | A | 75 | 39.538 | −7.913 | 7.476 | 1.00 | 42.35 | C |
| ATOM | 1211 | O | ILE | A | 75 | 39.350 | −7.353 | 6.451 | 1.00 | 39.45 | O |
| ATOM | 1212 | N | PRO | A | 76 | 38.576 | −8.592 | 8.099 | 1.00 | 43.86 | N |
| ATOM | 1213 | CA | PRO | A | 76 | 37.285 | −8.857 | 7.461 | 1.00 | 43.31 | C |
| ATOM | 1215 | CB | PRO | A | 76 | 36.796 | −10.049 | 8.210 | 1.00 | 43.97 | C |
| ATOM | 1218 | CG | PRO | A | 76 | 37.340 | −9.772 | 9.568 | 1.00 | 45.76 | C |
| ATOM | 1221 | CD | PRO | A | 76 | 38.691 | −9.247 | 9.406 | 1.00 | 44.45 | C |
| ATOM | 1224 | C | PRO | A | 76 | 36.324 | −7.728 | 7.568 | 1.00 | 42.78 | C |
| ATOM | 1225 | O | PRO | A | 76 | 35.396 | −7.831 | 8.333 | 1.00 | 45.44 | O |
| ATOM | 1226 | N | LEU | A | 77 | 36.535 | −6.697 | 6.748 | 1.00 | 41.49 | N |
| ATOM | 1228 | CA | LEU | A | 77 | 35.641 | −5.567 | 6.537 | 1.00 | 39.87 | C |
| ATOM | 1230 | CB | LEU | A | 77 | 36.546 | −4.409 | 6.198 | 1.00 | 38.77 | C |
| ATOM | 1233 | CG | LEU | A | 77 | 37.158 | −3.827 | 7.512 | 1.00 | 41.18 | C |
| ATOM | 1235 | CD1 | LEU | A | 77 | 38.215 | −2.809 | 7.271 | 1.00 | 41.61 | C |
| ATOM | 1239 | CD2 | LEU | A | 77 | 36.111 | −3.156 | 8.502 | 1.00 | 40.56 | C |
| ATOM | 1243 | C | LEU | A | 77 | 34.637 | −5.929 | 5.427 | 1.00 | 41.28 | C |
| ATOM | 1244 | O | LEU | A | 77 | 34.447 | −5.251 | 4.413 | 1.00 | 42.53 | O |
| ATOM | 1245 | N | GLU | A | 78 | 33.972 | −7.055 | 5.647 | 1.00 | 43.65 | N |
| ATOM | 1247 | CA | GLU | A | 78 | 33.051 | −7.679 | 4.726 | 1.00 | 44.72 | C |
| ATOM | 1249 | CB | GLU | A | 78 | 32.625 | −9.074 | 5.243 | 1.00 | 47.90 | C |
| ATOM | 1252 | CG | GLU | A | 78 | 33.638 | −10.261 | 5.125 | 1.00 | 49.04 | C |
| ATOM | 1255 | CD | GLU | A | 78 | 33.290 | −11.463 | 6.010 | 1.00 | 57.37 | C |
| ATOM | 1256 | OE1 | GLU | A | 78 | 32.198 | −11.438 | 6.698 | 1.00 | 60.60 | O |
| ATOM | 1257 | OE2 | GLU | A | 78 | 34.099 | −12.458 | 6.053 | 1.00 | 64.67 | O |
| ATOM | 1258 | C | GLU | A | 78 | 31.804 | −6.852 | 4.471 | 1.00 | 45.32 | C |
| ATOM | 1259 | O | GLU | A | 78 | 31.180 | −7.118 | 3.483 | 1.00 | 48.23 | O |
| ATOM | 1260 | N | ASN | A | 79 | 31.427 | −5.864 | 5.300 | 1.00 | 45.00 | N |
| ATOM | 1262 | CA | ASN | A | 79 | 30.167 | −5.128 | 5.064 | 1.00 | 45.06 | C |
| ATOM | 1264 | CB | ASN | A | 79 | 29.175 | −5.520 | 6.123 | 1.00 | 47.94 | C |
| ATOM | 1267 | CG | ASN | A | 79 | 28.893 | −7.032 | 6.112 | 1.00 | 46.44 | C |
| ATOM | 1268 | OD1 | ASN | A | 79 | 28.282 | −7.536 | 5.190 | 1.00 | 48.09 | O |
| ATOM | 1269 | ND2 | ASN | A | 79 | 29.350 | −7.723 | 7.101 | 1.00 | 45.47 | N |
| ATOM | 1272 | C | ASN | A | 79 | 30.263 | −3.572 | 4.936 | 1.00 | 44.03 | C |
| ATOM | 1273 | O | ASN | A | 79 | 29.243 | −2.882 | 4.734 | 1.00 | 44.31 | O |
| ATOM | 1274 | N | LEU | A | 80 | 31.505 | −3.086 | 4.968 | 1.00 | 41.14 | N |
| ATOM | 1276 | CA | LEU | A | 80 | 31.853 | −1.726 | 4.783 | 1.00 | 39.81 | C |
| ATOM | 1278 | CB | LEU | A | 80 | 33.381 | −1.607 | 4.910 | 1.00 | 35.95 | C |
| ATOM | 1281 | CG | LEU | A | 80 | 33.900 | −0.184 | 4.736 | 1.00 | 39.21 | C |
| ATOM | 1283 | CD1 | LEU | A | 80 | 33.164 | 0.825 | 5.606 | 1.00 | 37.81 | C |
| ATOM | 1287 | CD2 | LEU | A | 80 | 35.397 | 0.048 | 4.966 | 1.00 | 41.93 | C |
| ATOM | 1291 | C | LEU | A | 80 | 31.377 | −1.250 | 3.421 | 1.00 | 40.93 | C |
| ATOM | 1292 | O | LEU | A | 80 | 32.049 | −1.496 | 2.469 | 1.00 | 42.60 | O |
| ATOM | 1293 | N | GLN | A | 81 | 30.255 | −0.537 | 3.300 | 1.00 | 42.84 | N |
| ATOM | 1295 | CA | GLN | A | 81 | 29.796 | −0.098 | 1.984 | 1.00 | 43.93 | C |
| ATOM | 1297 | CB | GLN | A | 81 | 28.269 | 0.058 | 1.841 | 1.00 | 46.43 | C |
| ATOM | 1300 | CG | GLN | A | 81 | 27.410 | −0.770 | 2.822 | 1.00 | 53.59 | C |
| ATOM | 1303 | CD | GLN | A | 81 | 25.871 | −0.694 | 2.613 | 1.00 | 59.31 | C |
| ATOM | 1304 | OE1 | GLN | A | 81 | 25.397 | −0.526 | 1.466 | 1.00 | 64.23 | O |
| ATOM | 1305 | NE2 | GLN | A | 81 | 25.105 | −0.867 | 3.707 | 1.00 | 58.31 | N |
| ATOM | 1308 | C | GLN | A | 81 | 30.427 | 1.192 | 1.574 | 1.00 | 43.10 | C |
| ATOM | 1309 | O | GLN | A | 81 | 30.390 | 1.526 | 0.406 | 1.00 | 44.40 | O |
| ATOM | 1310 | N | ILE | A | 82 | 30.926 | 1.983 | 2.500 | 1.00 | 42.20 | N |
| ATOM | 1312 | CA | ILE | A | 82 | 31.227 | 3.374 | 2.148 | 1.00 | 41.78 | C |
| ATOM | 1314 | CB | ILE | A | 82 | 29.952 | 4.210 | 2.093 | 1.00 | 43.88 | C |
| ATOM | 1316 | CG1 | ILE | A | 82 | 30.226 | 5.693 | 1.936 | 1.00 | 44.99 | C |
| ATOM | 1319 | CD1 | ILE | A | 82 | 28.980 | 6.417 | 1.322 | 1.00 | 47.81 | C |
| ATOM | 1323 | CG2 | ILE | A | 82 | 29.181 | 4.105 | 3.300 | 1.00 | 46.56 | C |
| ATOM | 1327 | C | ILE | A | 82 | 32.123 | 4.000 | 3.099 | 1.00 | 40.42 | C |
| ATOM | 1328 | O | ILE | A | 82 | 31.890 | 3.965 | 4.277 | 1.00 | 40.10 | O |
| ATOM | 1329 | N | ILE | A | 83 | 33.149 | 4.590 | 2.559 | 1.00 | 40.78 | N |
| ATOM | 1331 | CA | ILE | A | 83 | 33.982 | 5.550 | 3.268 | 1.00 | 41.60 | C |
| ATOM | 1333 | CB | ILE | A | 83 | 35.472 | 5.198 | 2.997 | 1.00 | 40.44 | C |
| ATOM | 1335 | CG1 | ILE | A | 83 | 35.765 | 3.848 | 3.622 | 1.00 | 40.01 | C |
| ATOM | 1338 | CD1 | ILE | A | 83 | 36.743 | 3.034 | 2.815 | 1.00 | 42.41 | C |
| ATOM | 1342 | CG2 | ILE | A | 83 | 36.410 | 6.236 | 3.487 | 1.00 | 38.30 | C |
| ATOM | 1346 | C | ILE | A | 83 | 33.669 | 6.893 | 2.695 | 1.00 | 42.50 | C |
| ATOM | 1347 | O | ILE | A | 83 | 33.999 | 7.150 | 1.579 | 1.00 | 43.19 | O |
| ATOM | 1348 | N | ARG | A | 84 | 33.018 | 7.761 | 3.415 | 1.00 | 44.85 | N |
| ATOM | 1350 | CA | ARG | A | 84 | 32.956 | 9.181 | 2.954 | 1.00 | 47.16 | C |
| ATOM | 1352 | CB | ARG | A | 84 | 31.957 | 9.935 | 3.837 | 1.00 | 48.61 | C |
| ATOM | 1355 | CG | ARG | A | 84 | 30.620 | 9.238 | 4.016 | 1.00 | 46.73 | C |
| ATOM | 1358 | CD | ARG | A | 84 | 29.729 | 9.845 | 5.062 | 1.00 | 47.11 | C |
| ATOM | 1361 | NE | ARG | A | 84 | 28.625 | 8.937 | 5.218 | 1.00 | 49.65 | N |
| ATOM | 1363 | CZ | ARG | A | 84 | 27.542 | 8.873 | 4.460 | 1.00 | 52.43 | C |
| ATOM | 1364 | NH1 | ARG | A | 84 | 27.321 | 9.737 | 3.523 | 1.00 | 56.44 | N |
| ATOM | 1367 | NH2 | ARG | A | 84 | 26.651 | 7.905 | 4.649 | 1.00 | 56.44 | N |
| ATOM | 1370 | C | ARG | A | 84 | 34.366 | 9.791 | 3.133 | 1.00 | 47.81 | C |
| ATOM | 1371 | O | ARG | A | 84 | 35.119 | 9.296 | 3.931 | 1.00 | 49.72 | O |
| ATOM | 1372 | N | GLY | A | 85 | 34.798 | 10.845 | 2.512 | 1.00 | 49.39 | N |
| ATOM | 1374 | CA | GLY | A | 85 | 36.098 | 11.343 | 3.004 | 1.00 | 49.30 | C |
| ATOM | 1377 | C | GLY | A | 85 | 36.067 | 12.641 | 3.784 | 1.00 | 51.38 | C |
| ATOM | 1378 | O | GLY | A | 85 | 36.672 | 13.702 | 3.350 | 1.00 | 54.87 | O |
| ATOM | 1379 | N | ASN | A | 86 | 35.350 | 12.634 | 4.894 | 1.00 | 50.44 | N |
| ATOM | 1381 | CA | ASN | A | 86 | 35.247 | 13.909 | 5.616 | 1.00 | 53.16 | C |
| ATOM | 1383 | CB | ASN | A | 86 | 34.137 | 13.970 | 6.662 | 1.00 | 54.49 | C |
| ATOM | 1386 | CG | ASN | A | 86 | 32.855 | 13.473 | 6.133 | 1.00 | 56.57 | C |
| ATOM | 1387 | OD1 | ASN | A | 86 | 32.589 | 13.516 | 4.880 | 1.00 | 57.07 | O |
| ATOM | 1388 | ND2 | ASN | A | 86 | 32.041 | 12.937 | 7.053 | 1.00 | 58.10 | N |
| ATOM | 1391 | C | ASN | A | 86 | 36.544 | 14.299 | 6.243 | 1.00 | 52.27 | C |
| ATOM | 1392 | O | ASN | A | 86 | 36.736 | 15.441 | 6.387 | 1.00 | 54.97 | O |
| ATOM | 1393 | N | MET | A | 87 | 37.376 | 13.355 | 6.672 | 1.00 | 49.69 | N |
| ATOM | 1395 | CA | MET | A | 87 | 38.812 | 13.569 | 6.874 | 1.00 | 50.28 | C |
| ATOM | 1397 | CB | MET | A | 87 | 39.369 | 13.214 | 8.246 | 1.00 | 50.21 | C |
| ATOM | 1400 | CG | MET | A | 87 | 38.562 | 13.459 | 9.436 | 1.00 | 54.83 | C |
| ATOM | 1403 | SD | MET | A | 87 | 39.259 | 14.846 | 10.126 | 1.00 | 63.81 | S |
| ATOM | 1404 | CE | MET | A | 87 | 38.913 | 15.937 | 8.831 | 1.00 | 64.30 | C |
| ATOM | 1408 | C | MET | A | 87 | 39.563 | 12.647 | 5.990 | 1.00 | 48.04 | C |
| ATOM | 1409 | O | MET | A | 87 | 39.001 | 11.677 | 5.499 | 1.00 | 46.37 | O |
| ATOM | 1410 | N | TYR | A | 88 | 40.857 | 12.939 | 5.889 | 1.00 | 49.45 | N |
| ATOM | 1412 | CA | TYR | A | 88 | 41.828 | 12.188 | 5.123 | 1.00 | 49.57 | C |
| ATOM | 1414 | CB | TYR | A | 88 | 42.405 | 13.024 | 3.985 | 1.00 | 50.98 | C |
| ATOM | 1417 | CG | TYR | A | 88 | 41.429 | 13.553 | 2.930 | 1.00 | 53.15 | C |
| ATOM | 1418 | CD1 | TYR | A | 88 | 41.227 | 14.877 | 2.711 | 1.00 | 57.66 | C |
| ATOM | 1420 | CE1 | TYR | A | 88 | 40.400 | 15.281 | 1.763 | 1.00 | 56.97 | C |
| ATOM | 1422 | CZ | TYR | A | 88 | 39.767 | 14.410 | 0.984 | 1.00 | 52.93 | C |
| ATOM | 1423 | OH | TYR | A | 88 | 38.880 | 14.736 | 0.021 | 1.00 | 53.52 | O |
| ATOM | 1425 | CE2 | TYR | A | 88 | 39.952 | 13.177 | 1.150 | 1.00 | 52.34 | C |
| ATOM | 1427 | CD2 | TYR | A | 88 | 40.787 | 12.717 | 2.112 | 1.00 | 54.43 | C |
| ATOM | 1429 | C | TYR | A | 88 | 43.005 | 11.772 | 5.994 | 1.00 | 50.38 | C |
| ATOM | 1430 | O | TYR | A | 88 | 43.540 | 12.623 | 6.636 | 1.00 | 51.81 | O |
| ATOM | 1431 | N | TYR | A | 89 | 43.461 | 10.494 | 6.003 | 1.00 | 49.68 | N |
| ATOM | 1433 | CA | TYR | A | 89 | 44.851 | 10.226 | 6.458 | 1.00 | 51.12 | C |
| ATOM | 1435 | CB | TYR | A | 89 | 45.199 | 8.793 | 6.230 | 1.00 | 48.69 | C |
| ATOM | 1438 | CG | TYR | A | 89 | 46.550 | 8.377 | 6.698 | 1.00 | 49.97 | C |
| ATOM | 1439 | CD1 | TYR | A | 89 | 46.788 | 8.184 | 7.998 | 1.00 | 52.94 | C |
| ATOM | 1441 | CE1 | TYR | A | 89 | 48.046 | 7.787 | 8.456 | 1.00 | 54.76 | C |
| ATOM | 1443 | CZ | TYR | A | 89 | 49.063 | 7.565 | 7.595 | 1.00 | 53.29 | C |
| ATOM | 1444 | OH | TYR | A | 89 | 50.254 | 7.164 | 8.142 | 1.00 | 54.86 | O |
| ATOM | 1446 | CE2 | TYR | A | 89 | 48.855 | 7.728 | 6.279 | 1.00 | 52.09 | C |
| ATOM | 1448 | CD2 | TYR | A | 89 | 47.581 | 8.133 | 5.832 | 1.00 | 52.11 | C |
| ATOM | 1450 | C | TYR | A | 89 | 45.758 | 11.121 | 5.592 | 1.00 | 54.75 | C |
| ATOM | 1451 | O | TYR | A | 89 | 45.311 | 11.553 | 4.499 | 1.00 | 57.07 | O |
| ATOM | 1452 | N | GLU | A | 90 | 46.990 | 11.416 | 6.012 | 1.00 | 56.38 | N |
| ATOM | 1454 | CA | GLU | A | 90 | 47.978 | 12.129 | 5.116 | 1.00 | 58.43 | C |
| ATOM | 1456 | CB | GLU | A | 90 | 49.394 | 11.443 | 5.224 | 1.00 | 60.02 | C |
| ATOM | 1459 | CG | GLU | A | 90 | 49.655 | 10.346 | 4.183 | 1.00 | 63.93 | C |
| ATOM | 1462 | CD | GLU | A | 90 | 51.108 | 9.763 | 4.087 | 1.00 | 72.04 | C |
| ATOM | 1463 | OE1 | GLU | A | 90 | 52.082 | 10.496 | 4.526 | 1.00 | 73.67 | O |
| ATOM | 1464 | OE2 | GLU | A | 90 | 51.246 | 8.551 | 3.534 | 1.00 | 70.25 | O |
| ATOM | 1465 | C | GLU | A | 90 | 47.584 | 12.478 | 3.607 | 1.00 | 56.72 | C |
| ATOM | 1466 | O | GLU | A | 90 | 47.069 | 11.643 | 2.909 | 1.00 | 53.74 | O |
| ATOM | 1467 | N | ASN | A | 91 | 47.844 | 13.712 | 3.136 | 1.00 | 59.85 | N |
| ATOM | 1469 | CA | ASN | A | 91 | 47.846 | 14.086 | 1.691 | 1.00 | 61.10 | C |
| ATOM | 1471 | CB | ASN | A | 91 | 48.956 | 13.309 | 0.852 | 1.00 | 62.38 | C |
| ATOM | 1474 | CG | ASN | A | 91 | 50.403 | 13.342 | 1.452 | 1.00 | 66.43 | C |
| ATOM | 1475 | OD1 | ASN | A | 91 | 51.269 | 14.148 | 1.040 | 1.00 | 66.35 | O |
| ATOM | 1476 | ND2 | ASN | A | 91 | 50.692 | 12.379 | 2.344 | 1.00 | 68.77 | N |
| ATOM | 1479 | C | ASN | A | 91 | 46.511 | 13.735 | 1.008 | 1.00 | 57.87 | C |
| ATOM | 1480 | O | ASN | A | 91 | 46.489 | 13.144 | −0.104 | 1.00 | 58.73 | O |
| ATOM | 1481 | N | SER | A | 92 | 45.373 | 13.936 | 1.639 | 1.00 | 54.13 | N |
| ATOM | 1483 | CA | SER | A | 92 | 44.142 | 13.510 | 0.901 | 1.00 | 50.01 | C |
| ATOM | 1485 | CB | SER | A | 92 | 43.991 | 14.441 | −0.308 | 1.00 | 53.39 | C |
| ATOM | 1488 | OG | SER | A | 92 | 44.397 | 13.795 | −1.479 | 1.00 | 55.51 | O |
| ATOM | 1490 | C | SER | A | 92 | 43.861 | 11.994 | 0.390 | 1.00 | 45.90 | C |
| ATOM | 1491 | O | SER | A | 92 | 43.244 | 11.776 | −0.632 | 1.00 | 45.63 | O |
| ATOM | 1492 | N | TYR | A | 93 | 44.238 | 11.012 | 1.185 | 1.00 | 44.30 | N |
| ATOM | 1494 | CA | TYR | A | 93 | 43.804 | 9.637 | 0.991 | 1.00 | 42.94 | C |
| ATOM | 1496 | CB | TYR | A | 93 | 44.955 | 8.620 | 1.158 | 1.00 | 41.72 | C |
| ATOM | 1499 | CG | TYR | A | 93 | 46.166 | 8.787 | 0.278 | 1.00 | 43.87 | C |
| ATOM | 1500 | CD1 | TYR | A | 93 | 47.407 | 9.146 | 0.829 | 1.00 | 46.10 | C |
| ATOM | 1502 | CE1 | TYR | A | 93 | 48.541 | 9.327 | 0.012 | 1.00 | 50.76 | C |
| ATOM | 1504 | CZ | TYR | A | 93 | 48.439 | 9.139 | −1.371 | 1.00 | 48.97 | C |
| ATOM | 1505 | OH | TYR | A | 93 | 49.527 | 9.284 | −2.159 | 1.00 | 51.85 | O |
| ATOM | 1507 | CE2 | TYR | A | 93 | 47.228 | 8.775 | −1.937 | 1.00 | 47.04 | C |
| ATOM | 1509 | CD2 | TYR | A | 93 | 46.087 | 8.588 | −1.104 | 1.00 | 44.48 | C |
| ATOM | 1511 | C | TYR | A | 93 | 42.754 | 9.357 | 2.068 | 1.00 | 42.52 | C |
| ATOM | 1512 | O | TYR | A | 93 | 43.009 | 9.539 | 3.289 | 1.00 | 42.34 | O |
| ATOM | 1513 | N | ALA | A | 94 | 41.585 | 8.947 | 1.581 | 1.00 | 42.12 | N |
| ATOM | 1515 | CA | ALA | A | 94 | 40.467 | 8.458 | 2.386 | 1.00 | 41.29 | C |
| ATOM | 1517 | CB | ALA | A | 94 | 39.124 | 8.712 | 1.688 | 1.00 | 41.93 | C |
| ATOM | 1521 | C | ALA | A | 94 | 40.538 | 7.046 | 2.741 | 1.00 | 36.58 | C |
| ATOM | 1522 | O | ALA | A | 94 | 39.832 | 6.652 | 3.648 | 1.00 | 38.88 | O |
| ATOM | 1523 | N | LEU | A | 95 | 41.322 | 6.287 | 1.976 | 1.00 | 37.35 | N |
| ATOM | 1525 | CA | LEU | A | 95 | 41.677 | 4.889 | 2.345 | 1.00 | 36.53 | C |
| ATOM | 1527 | CB | LEU | A | 95 | 40.838 | 3.878 | 1.575 | 1.00 | 35.59 | C |
| ATOM | 1530 | CG | LEU | A | 95 | 41.249 | 2.405 | 1.730 | 1.00 | 38.72 | C |
| ATOM | 1532 | CD1 | LEU | A | 95 | 40.952 | 1.856 | 3.092 | 1.00 | 38.41 | C |
| ATOM | 1536 | CD2 | LEU | A | 95 | 40.553 | 1.538 | 0.725 | 1.00 | 40.76 | C |
| ATOM | 1540 | C | LEU | A | 95 | 43.155 | 4.620 | 2.097 | 1.00 | 36.96 | C |
| ATOM | 1541 | O | LEU | A | 95 | 43.702 | 4.902 | 1.040 | 1.00 | 36.85 | O |
| ATOM | 1542 | N | ALA | A | 96 | 43.811 | 4.030 | 3.064 | 1.00 | 36.75 | N |
| ATOM | 1544 | CA | ALA | A | 96 | 45.256 | 3.954 | 2.964 | 1.00 | 39.78 | C |
| ATOM | 1546 | CB | ALA | A | 96 | 45.925 | 5.164 | 3.605 | 1.00 | 41.49 | C |
| ATOM | 1550 | C | ALA | A | 96 | 45.666 | 2.689 | 3.629 | 1.00 | 40.17 | C |
| ATOM | 1551 | O | ALA | A | 96 | 45.306 | 2.465 | 4.723 | 1.00 | 39.98 | O |
| ATOM | 1552 | N | VAL | A | 97 | 46.352 | 1.827 | 2.908 | 1.00 | 41.75 | N |
| ATOM | 1554 | CA | VAL | A | 97 | 46.585 | 0.484 | 3.341 | 1.00 | 42.09 | C |
| ATOM | 1556 | CB | VAL | A | 97 | 45.788 | −0.476 | 2.463 | 1.00 | 41.49 | C |
| ATOM | 1558 | CG1 | VAL | A | 97 | 46.026 | −1.918 | 2.852 | 1.00 | 42.14 | C |
| ATOM | 1562 | CG2 | VAL | A | 97 | 44.323 | −0.177 | 2.615 | 1.00 | 41.22 | C |
| ATOM | 1566 | C | VAL | A | 97 | 48.086 | 0.352 | 3.161 | 1.00 | 44.34 | C |
| ATOM | 1567 | O | VAL | A | 97 | 48.554 | 0.134 | 2.051 | 1.00 | 45.40 | O |
| ATOM | 1568 | N | LEU | A | 98 | 48.818 | 0.528 | 4.272 | 1.00 | 45.17 | N |
| ATOM | 1570 | CA | LEU | A | 98 | 50.276 | 0.703 | 4.325 | 1.00 | 46.32 | C |
| ATOM | 1572 | CB | LEU | A | 98 | 50.596 | 2.056 | 4.941 | 1.00 | 47.45 | C |
| ATOM | 1575 | CG | LEU | A | 98 | 49.900 | 3.267 | 4.427 | 1.00 | 46.46 | C |
| ATOM | 1577 | CD1 | LEU | A | 98 | 50.711 | 4.432 | 4.845 | 1.00 | 49.07 | C |
| ATOM | 1581 | CD2 | LEU | A | 98 | 49.904 | 3.181 | 2.962 | 1.00 | 49.37 | C |
| ATOM | 1585 | C | LEU | A | 98 | 51.052 | −0.301 | 5.190 | 1.00 | 46.58 | C |
| ATOM | 1586 | O | LEU | A | 98 | 50.744 | −0.562 | 6.346 | 1.00 | 44.34 | O |
| ATOM | 1587 | N | SER | A | 99 | 52.121 | −0.774 | 4.585 | 1.00 | 48.54 | N |
| ATOM | 1589 | CA | SER | A | 99 | 53.196 | −1.505 | 5.215 | 1.00 | 51.61 | C |
| ATOM | 1591 | CB | SER | A | 99 | 54.303 | −0.529 | 5.593 | 1.00 | 54.70 | C |
| ATOM | 1594 | OG | SER | A | 99 | 53.951 | 0.131 | 6.737 | 1.00 | 56.30 | O |
| ATOM | 1596 | C | SER | A | 99 | 52.781 | −2.478 | 6.314 | 1.00 | 50.51 | C |
| ATOM | 1597 | O | SER | A | 99 | 53.385 | −2.555 | 7.367 | 1.00 | 51.44 | O |
| ATOM | 1598 | N | ASN | A | 100 | 51.810 | −3.301 | 5.909 | 1.00 | 48.91 | N |
| ATOM | 1600 | CA | ASN | A | 100 | 51.163 | −4.336 | 6.673 | 1.00 | 48.16 | C |
| ATOM | 1602 | CB | ASN | A | 100 | 49.731 | −4.596 | 6.150 | 1.00 | 45.34 | C |
| ATOM | 1605 | CG | ASN | A | 100 | 48.753 | −3.488 | 6.537 | 1.00 | 43.98 | C |
| ATOM | 1606 | OD1 | ASN | A | 100 | 48.493 | −3.220 | 7.752 | 1.00 | 44.49 | O |
| ATOM | 1607 | ND2 | ASN | A | 100 | 48.219 | −2.820 | 5.529 | 1.00 | 40.05 | N |
| ATOM | 1610 | C | ASN | A | 100 | 51.948 | −5.611 | 6.590 | 1.00 | 50.03 | C |
| ATOM | 1611 | O | ASN | A | 100 | 51.466 | −6.581 | 6.059 | 1.00 | 49.67 | O |
| ATOM | 1612 | N | TYR | A | 101 | 53.167 | −5.561 | 7.095 | 1.00 | 52.63 | N |
| ATOM | 1614 | CA | TYR | A | 101 | 54.057 | −6.682 | 7.206 | 1.00 | 55.81 | C |
| ATOM | 1616 | CB | TYR | A | 101 | 54.935 | −6.876 | 5.957 | 1.00 | 57.44 | C |
| ATOM | 1619 | CG | TYR | A | 101 | 55.910 | −5.739 | 5.711 | 1.00 | 61.09 | C |
| ATOM | 1620 | CD1 | TYR | A | 101 | 57.282 | −5.915 | 5.822 | 1.00 | 65.26 | C |
| ATOM | 1622 | CE1 | TYR | A | 101 | 58.178 | −4.835 | 5.598 | 1.00 | 67.71 | C |
| ATOM | 1624 | CZ | TYR | A | 101 | 57.689 | −3.571 | 5.279 | 1.00 | 66.52 | C |
| ATOM | 1625 | OH | TYR | A | 101 | 58.504 | −2.489 | 5.080 | 1.00 | 66.74 | O |
| ATOM | 1627 | CE2 | TYR | A | 101 | 56.339 | −3.370 | 5.169 | 1.00 | 63.37 | C |
| ATOM | 1629 | CD2 | TYR | A | 101 | 55.450 | −4.453 | 5.380 | 1.00 | 61.79 | C |
| ATOM | 1631 | C | TYR | A | 101 | 54.922 | −6.375 | 8.399 | 1.00 | 59.02 | C |
| ATOM | 1632 | O | TYR | A | 101 | 54.849 | −5.281 | 8.984 | 1.00 | 59.11 | O |
| ATOM | 1633 | N | ASP | A | 102 | 55.740 | −7.351 | 8.775 | 1.00 | 62.99 | N |
| ATOM | 1635 | CA | ASP | A | 102 | 56.695 | −7.200 | 9.858 | 1.00 | 66.01 | C |
| ATOM | 1637 | CB | ASP | A | 102 | 56.193 | −7.849 | 11.169 | 1.00 | 66.35 | C |
| ATOM | 1640 | CG | ASP | A | 102 | 56.362 | −9.374 | 11.239 | 1.00 | 70.03 | C |
| ATOM | 1641 | OD1 | ASP | A | 102 | 56.452 | −10.092 | 10.212 | 1.00 | 73.62 | O |
| ATOM | 1642 | OD2 | ASP | A | 102 | 56.400 | −9.961 | 12.340 | 1.00 | 72.18 | O |
| ATOM | 1643 | C | ASP | A | 102 | 58.069 | −7.681 | 9.459 | 1.00 | 69.88 | C |
| ATOM | 1644 | O | ASP | A | 102 | 58.284 | −8.255 | 8.390 | 1.00 | 69.60 | O |
| ATOM | 1645 | N | ALA | A | 103 | 59.005 | −7.382 | 10.340 | 1.00 | 73.77 | N |
| ATOM | 1647 | CA | ALA | A | 103 | 60.390 | −7.786 | 10.188 | 1.00 | 78.79 | C |
| ATOM | 1649 | CB | ALA | A | 103 | 61.234 | −7.388 | 11.471 | 1.00 | 82.44 | C |
| ATOM | 1653 | C | ALA | A | 103 | 60.469 | −9.265 | 9.936 | 1.00 | 80.28 | C |
| ATOM | 1654 | O | ALA | A | 103 | 61.349 | −9.677 | 9.202 | 1.00 | 83.58 | O |
| ATOM | 1655 | N | ASN | A | 104 | 59.535 | −10.036 | 10.519 | 1.00 | 78.84 | N |
| ATOM | 1657 | CA | ASN | A | 104 | 59.562 | −11.517 | 10.518 | 1.00 | 81.30 | C |
| ATOM | 1659 | CB | ASN | A | 104 | 58.700 | −12.058 | 11.667 | 1.00 | 80.83 | C |
| ATOM | 1662 | CG | ASN | A | 104 | 59.503 | −12.368 | 12.896 | 1.00 | 85.11 | C |
| ATOM | 1663 | OD1 | ASN | A | 104 | 60.124 | −13.432 | 12.973 | 1.00 | 88.51 | O |
| ATOM | 1664 | ND2 | ASN | A | 104 | 59.490 | −11.439 | 13.881 | 1.00 | 83.35 | N |
| ATOM | 1667 | C | ASN | A | 104 | 59.156 | −12.295 | 9.262 | 1.00 | 80.02 | C |
| ATOM | 1668 | O | ASN | A | 104 | 59.059 | −13.525 | 9.360 | 1.00 | 81.89 | O |
| ATOM | 1669 | N | ALA | A | 105 | 58.967 | −11.613 | 8.120 | 1.00 | 77.36 | N |
| ATOM | 1671 | CA | ALA | A | 105 | 58.482 | −12.224 | 6.874 | 1.00 | 76.38 | C |
| ATOM | 1673 | CB | ALA | A | 105 | 59.452 | −13.356 | 6.374 | 1.00 | 80.34 | C |
| ATOM | 1677 | C | ALA | A | 105 | 57.000 | −12.731 | 6.961 | 1.00 | 73.17 | C |
| ATOM | 1678 | O | ALA | A | 105 | 56.718 | −13.889 | 6.700 | 1.00 | 73.97 | O |
| ATOM | 1679 | N | THR | A | 106 | 56.081 | −11.824 | 7.298 | 1.00 | 69.66 | N |
| ATOM | 1681 | CA | THR | A | 106 | 54.660 | −12.097 | 7.532 | 1.00 | 66.80 | C |
| ATOM | 1683 | CB | THR | A | 106 | 54.420 | −12.371 | 9.047 | 1.00 | 68.37 | C |
| ATOM | 1685 | OG1 | THR | A | 106 | 55.307 | −13.390 | 9.523 | 1.00 | 72.58 | O |
| ATOM | 1687 | CG2 | THR | A | 106 | 52.978 | −12.913 | 9.329 | 1.00 | 67.58 | C |
| ATOM | 1691 | C | THR | A | 106 | 53.902 | −10.812 | 7.270 | 1.00 | 62.45 | C |
| ATOM | 1692 | O | THR | A | 106 | 54.416 | −9.762 | 7.574 | 1.00 | 62.25 | O |
| ATOM | 1693 | N | GLY | A | 107 | 52.673 | −10.886 | 6.782 | 1.00 | 58.72 | N |
| ATOM | 1695 | CA | GLY | A | 107 | 51.864 | −9.712 | 6.675 | 1.00 | 55.00 | C |
| ATOM | 1698 | C | GLY | A | 107 | 50.400 | −9.905 | 6.384 | 1.00 | 53.15 | C |
| ATOM | 1699 | O | GLY | A | 107 | 49.843 | −10.953 | 6.433 | 1.00 | 53.80 | O |
| ATOM | 1700 | N | LEU | A | 108 | 49.751 | −8.814 | 6.049 | 1.00 | 52.41 | N |
| ATOM | 1702 | CA | LEU | A | 108 | 48.340 | −8.814 | 5.782 | 1.00 | 50.68 | C |
| ATOM | 1704 | CB | LEU | A | 108 | 47.813 | −7.395 | 5.753 | 1.00 | 47.39 | C |
| ATOM | 1707 | CG | LEU | A | 108 | 46.336 | −7.409 | 5.418 | 1.00 | 45.14 | C |
| ATOM | 1709 | CD1 | LEU | A | 108 | 45.449 | −8.110 | 6.475 | 1.00 | 45.32 | C |
| ATOM | 1713 | CD2 | LEU | A | 108 | 45.931 | −5.996 | 5.325 | 1.00 | 43.71 | C |
| ATOM | 1717 | C | LEU | A | 108 | 48.153 | −9.472 | 4.422 | 1.00 | 52.50 | C |
| ATOM | 1718 | O | LEU | A | 108 | 48.849 | −9.151 | 3.447 | 1.00 | 55.00 | O |
| ATOM | 1719 | N | LYS | A | 109 | 47.179 | −10.355 | 4.355 | 1.00 | 52.74 | N |
| ATOM | 1721 | CA | LYS | A | 109 | 46.955 | −11.238 | 3.229 | 1.00 | 53.15 | C |
| ATOM | 1723 | CB | LYS | A | 109 | 47.638 | −12.559 | 3.644 | 1.00 | 55.47 | C |
| ATOM | 1726 | CG | LYS | A | 109 | 47.261 | −13.758 | 2.850 | 1.00 | 58.35 | C |
| ATOM | 1729 | CD | LYS | A | 109 | 47.898 | −15.038 | 3.348 | 1.00 | 64.60 | C |
| ATOM | 1732 | CE | LYS | A | 109 | 47.531 | −16.247 | 2.344 | 1.00 | 68.67 | C |
| ATOM | 1735 | NZ | LYS | A | 109 | 48.322 | −17.517 | 2.627 | 1.00 | 71.64 | N |
| ATOM | 1739 | C | LYS | A | 109 | 45.472 | −11.376 | 3.326 | 1.00 | 51.57 | C |
| ATOM | 1740 | O | LYS | A | 109 | 45.043 | −11.938 | 4.301 | 1.00 | 55.49 | O |
| ATOM | 1741 | N | GLU | A | 110 | 44.629 | −10.868 | 2.482 | 1.00 | 48.33 | N |
| ATOM | 1743 | CA | GLU | A | 110 | 43.162 | −10.946 | 2.841 | 1.00 | 47.27 | C |
| ATOM | 1745 | CB | GLU | A | 110 | 42.668 | −12.235 | 3.564 | 1.00 | 48.52 | C |
| ATOM | 1748 | CG | GLU | A | 110 | 42.930 | −13.571 | 2.828 | 1.00 | 52.86 | C |
| ATOM | 1751 | CD | GLU | A | 110 | 42.513 | −14.864 | 3.570 | 1.00 | 55.78 | C |
| ATOM | 1752 | OE1 | GLU | A | 110 | 41.488 | −14.854 | 4.321 | 1.00 | 53.35 | O |
| ATOM | 1753 | OE2 | GLU | A | 110 | 43.190 | −15.927 | 3.337 | 1.00 | 63.72 | O |
| ATOM | 1754 | C | GLU | A | 110 | 42.519 | −9.791 | 3.590 | 1.00 | 44.72 | C |
| ATOM | 1755 | O | GLU | A | 110 | 42.327 | −9.843 | 4.769 | 1.00 | 44.16 | O |
| ATOM | 1756 | N | LEU | A | 111 | 42.058 | −8.851 | 2.780 | 1.00 | 42.81 | N |
| ATOM | 1758 | CA | LEU | A | 111 | 41.322 | −7.684 | 3.153 | 1.00 | 41.48 | C |
| ATOM | 1760 | CB | LEU | A | 111 | 42.164 | −6.445 | 2.787 | 1.00 | 40.76 | C |
| ATOM | 1763 | CG | LEU | A | 111 | 41.578 | −5.068 | 2.935 | 1.00 | 39.62 | C |
| ATOM | 1765 | CD1 | LEU | A | 111 | 41.011 | −4.863 | 4.331 | 1.00 | 41.72 | C |
| ATOM | 1769 | CD2 | LEU | A | 111 | 42.691 | −4.060 | 2.710 | 1.00 | 39.92 | C |
| ATOM | 1773 | C | LEU | A | 111 | 40.103 | −7.645 | 2.272 | 1.00 | 41.38 | C |
| ATOM | 1774 | O | LEU | A | 111 | 40.014 | −6.823 | 1.395 | 1.00 | 42.26 | O |
| ATOM | 1775 | N | PRO | A | 112 | 39.179 | −8.521 | 2.525 | 1.00 | 41.64 | N |
| ATOM | 1776 | CA | PRO | A | 112 | 38.060 | −8.786 | 1.640 | 1.00 | 42.53 | C |
| ATOM | 1778 | CB | PRO | A | 112 | 37.638 | −10.175 | 2.115 | 1.00 | 44.60 | C |
| ATOM | 1781 | CG | PRO | A | 112 | 37.805 | −10.066 | 3.588 | 1.00 | 43.64 | C |
| ATOM | 1784 | CD | PRO | A | 112 | 39.114 | −9.318 | 3.760 | 1.00 | 43.26 | C |
| ATOM | 1787 | C | PRO | A | 112 | 36.845 | −7.849 | 1.760 | 1.00 | 42.22 | C |
| ATOM | 1788 | O | PRO | A | 112 | 35.765 | −8.273 | 2.247 | 1.00 | 43.92 | O |
| ATOM | 1789 | N | MET | A | 113 | 36.976 | −6.650 | 1.225 | 1.00 | 41.57 | N |
| ATOM | 1791 | CA | MET | A | 113 | 35.973 | −5.586 | 1.353 | 1.00 | 41.49 | C |
| ATOM | 1793 | CB | MET | A | 113 | 36.676 | −4.230 | 1.468 | 1.00 | 40.58 | C |
| ATOM | 1796 | CG | MET | A | 113 | 37.824 | −4.291 | 2.459 | 1.00 | 41.70 | C |
| ATOM | 1799 | SD | MET | A | 113 | 38.645 | −2.730 | 2.961 | 1.00 | 44.40 | S |
| ATOM | 1800 | CE | MET | A | 113 | 38.085 | −1.709 | 1.780 | 1.00 | 48.11 | C |
| ATOM | 1804 | C | MET | A | 113 | 35.088 | −5.639 | 0.146 | 1.00 | 42.26 | C |
| ATOM | 1805 | O | MET | A | 113 | 35.080 | −4.740 | −0.708 | 1.00 | 42.73 | O |
| ATOM | 1806 | N | ARG | A | 114 | 34.344 | −6.726 | 0.053 | 1.00 | 42.70 | N |
| ATOM | 1808 | CA | ARG | A | 114 | 33.513 | −6.960 | −1.108 | 1.00 | 43.40 | C |
| ATOM | 1810 | CB | ARG | A | 114 | 33.165 | −8.423 | −1.136 | 1.00 | 45.30 | C |
| ATOM | 1813 | CG | ARG | A | 114 | 32.163 | −8.694 | −0.123 | 1.00 | 48.87 | C |
| ATOM | 1816 | CD | ARG | A | 114 | 32.431 | −9.967 | 0.498 | 1.00 | 54.37 | C |
| ATOM | 1819 | NE | ARG | A | 114 | 31.551 | −10.186 | 1.603 | 1.00 | 56.34 | N |
| ATOM | 1821 | CZ | ARG | A | 114 | 31.514 | −11.297 | 2.263 | 1.00 | 55.48 | C |
| ATOM | 1822 | NH1 | ARG | A | 114 | 32.344 | −12.304 | 1.982 | 1.00 | 51.37 | N |
| ATOM | 1825 | NH2 | ARG | A | 114 | 30.674 | −11.331 | 3.277 | 1.00 | 60.58 | N |
| ATOM | 1828 | C | ARG | A | 114 | 32.209 | −6.193 | −1.176 | 1.00 | 42.43 | C |
| ATOM | 1829 | O | ARG | A | 114 | 31.422 | −6.405 | −2.096 | 1.00 | 43.72 | O |
| ATOM | 1830 | N | ASN | A | 115 | 31.936 | −5.385 | −0.165 | 1.00 | 41.74 | N |
| ATOM | 1832 | CA | ASN | A | 115 | 30.800 | −4.474 | −0.183 | 1.00 | 42.25 | C |
| ATOM | 1834 | CB | ASN | A | 115 | 29.944 | −4.676 | 1.039 | 1.00 | 43.29 | C |
| ATOM | 1837 | CG | ASN | A | 115 | 29.106 | −5.845 | 0.914 | 1.00 | 44.56 | C |
| ATOM | 1838 | OD1 | ASN | A | 115 | 28.419 | −6.041 | −0.040 | 1.00 | 47.36 | O |
| ATOM | 1839 | ND2 | ASN | A | 115 | 29.181 | −6.668 | 1.865 | 1.00 | 52.65 | N |
| ATOM | 1842 | C | ASN | A | 115 | 31.177 | −3.013 | −0.329 | 1.00 | 40.39 | C |
| ATOM | 1843 | O | ASN | A | 115 | 30.332 | −2.226 | −0.638 | 1.00 | 41.23 | O |
| ATOM | 1844 | N | LEU | A | 116 | 32.440 | −2.683 | −0.159 | 1.00 | 38.95 | N |
| ATOM | 1846 | CA | LEU | A | 116 | 32.917 | −1.357 | −0.384 | 1.00 | 39.18 | C |
| ATOM | 1848 | CB | LEU | A | 116 | 34.415 | −1.245 | −0.029 | 1.00 | 37.99 | C |
| ATOM | 1851 | CG | LEU | A | 116 | 34.961 | 0.166 | −0.304 | 1.00 | 38.33 | C |
| ATOM | 1853 | CD1 | LEU | A | 116 | 34.239 | 1.149 | 0.563 | 1.00 | 39.42 | C |
| ATOM | 1857 | CD2 | LEU | A | 116 | 36.421 | 0.263 | −0.035 | 1.00 | 38.20 | C |
| ATOM | 1861 | C | LEU | A | 116 | 32.611 | −0.853 | −1.839 | 1.00 | 40.77 | C |
| ATOM | 1862 | O | LEU | A | 116 | 33.220 | −1.240 | −2.811 | 1.00 | 41.13 | O |
| ATOM | 1863 | N | GLN | A | 117 | 31.641 | 0.020 | −1.950 | 1.00 | 41.94 | N |
| ATOM | 1865 | CA | GLN | A | 117 | 31.185 | 0.433 | −3.228 | 1.00 | 44.45 | C |
| ATOM | 1867 | CB | GLN | A | 117 | 29.790 | −0.099 | −3.459 | 1.00 | 46.41 | C |
| ATOM | 1870 | CG | GLN | A | 117 | 29.915 | −1.437 | −4.132 | 1.00 | 50.06 | C |
| ATOM | 1873 | CD | GLN | A | 117 | 28.659 | −2.188 | −4.162 | 1.00 | 54.73 | C |
| ATOM | 1874 | OE1 | GLN | A | 117 | 27.898 | −2.178 | −3.185 | 1.00 | 56.09 | O |
| ATOM | 1875 | NE2 | GLN | A | 117 | 28.436 | −2.883 | −5.252 | 1.00 | 56.36 | N |
| ATOM | 1878 | C | GLN | A | 117 | 31.319 | 1.908 | −3.539 | 1.00 | 44.91 | C |
| ATOM | 1879 | O | GLN | A | 117 | 31.561 | 2.178 | −4.686 | 1.00 | 47.48 | O |
| ATOM | 1880 | N | GLU | A | 118 | 31.246 | 2.824 | −2.567 | 1.00 | 43.90 | N |
| ATOM | 1882 | CA | GLU | A | 118 | 31.630 | 4.203 | −2.827 | 1.00 | 44.87 | C |
| ATOM | 1884 | CB | GLU | A | 118 | 30.338 | 5.058 | −3.007 | 1.00 | 48.13 | C |
| ATOM | 1887 | CG | GLU | A | 118 | 30.326 | 6.583 | −3.040 | 1.00 | 50.77 | C |
| ATOM | 1890 | CD | GLU | A | 118 | 30.632 | 7.244 | −4.365 | 1.00 | 56.86 | C |
| ATOM | 1891 | OE1 | GLU | A | 118 | 31.202 | 6.589 | −5.234 | 1.00 | 65.98 | O |
| ATOM | 1892 | OE2 | GLU | A | 118 | 30.462 | 8.468 | −4.540 | 1.00 | 58.62 | O |
| ATOM | 1893 | C | GLU | A | 118 | 32.606 | 4.742 | −1.785 | 1.00 | 42.40 | C |
| ATOM | 1894 | O | GLU | A | 118 | 32.534 | 4.414 | −0.641 | 1.00 | 40.40 | O |
| ATOM | 1895 | N | ILE | A | 119 | 33.550 | 5.555 | −2.272 | 1.00 | 42.58 | N |
| ATOM | 1897 | CA | ILE | A | 119 | 34.382 | 6.462 | −1.481 | 1.00 | 41.49 | C |
| ATOM | 1899 | CB | ILE | A | 119 | 35.884 | 6.155 | −1.688 | 1.00 | 40.87 | C |
| ATOM | 1901 | CG1 | ILE | A | 119 | 36.283 | 4.839 | −1.043 | 1.00 | 40.15 | C |
| ATOM | 1904 | CD1 | ILE | A | 119 | 37.837 | 4.504 | −1.293 | 1.00 | 40.31 | C |
| ATOM | 1908 | CG2 | ILE | A | 119 | 36.808 | 7.251 | −1.096 | 1.00 | 40.44 | C |
| ATOM | 1912 | C | ILE | A | 119 | 34.012 | 7.873 | −1.961 | 1.00 | 41.94 | C |
| ATOM | 1913 | O | ILE | A | 119 | 34.441 | 8.325 | −3.039 | 1.00 | 44.53 | O |
| ATOM | 1914 | N | LEU | A | 120 | 33.182 | 8.572 | −1.200 | 1.00 | 42.54 | N |
| ATOM | 1916 | CA | LEU | A | 120 | 32.619 | 9.831 | −1.705 | 1.00 | 46.08 | C |
| ATOM | 1918 | CB | LEU | A | 120 | 31.798 | 10.529 | −0.657 | 1.00 | 47.62 | C |
| ATOM | 1921 | CG | LEU | A | 120 | 30.428 | 10.004 | −0.329 | 1.00 | 50.88 | C |
| ATOM | 1923 | CD1 | LEU | A | 120 | 29.763 | 11.116 | 0.529 | 1.00 | 54.37 | C |
| ATOM | 1927 | CD2 | LEU | A | 120 | 29.652 | 9.701 | −1.610 | 1.00 | 50.49 | C |
| ATOM | 1931 | C | LEU | A | 120 | 33.658 | 10.830 | −2.141 | 1.00 | 46.27 | C |
| ATOM | 1932 | O | LEU | A | 120 | 33.462 | 11.522 | −3.112 | 1.00 | 48.50 | O |
| ATOM | 1933 | N | HIS | A | 121 | 34.737 | 10.906 | −1.361 | 1.00 | 45.34 | N |
| ATOM | 1935 | CA | HIS | A | 121 | 35.891 | 11.734 | −1.632 | 1.00 | 45.38 | C |
| ATOM | 1937 | CB | HIS | A | 121 | 35.889 | 12.961 | −0.796 | 1.00 | 47.60 | C |
| ATOM | 1940 | CG | HIS | A | 121 | 34.582 | 13.616 | −0.646 | 1.00 | 47.72 | C |
| ATOM | 1941 | ND1 | HIS | A | 121 | 34.347 | 14.891 | −1.097 | 1.00 | 49.72 | N |
| ATOM | 1943 | CE1 | HIS | A | 121 | 33.129 | 15.251 | −0.758 | 1.00 | 50.90 | C |
| ATOM | 1945 | NE2 | HIS | A | 121 | 32.577 | 14.277 | −0.081 | 1.00 | 49.78 | N |
| ATOM | 1947 | CD2 | HIS | A | 121 | 33.480 | 13.248 | 0.038 | 1.00 | 47.09 | C |
| ATOM | 1949 | C | HIS | A | 121 | 37.208 | 11.131 | −1.263 | 1.00 | 44.24 | C |
| ATOM | 1950 | O | HIS | A | 121 | 37.331 | 10.470 | −0.247 | 1.00 | 43.40 | O |
| ATOM | 1951 | N | GLY | A | 122 | 38.215 | 11.496 | −2.036 | 1.00 | 46.45 | N |
| ATOM | 1953 | CA | GLY | A | 122 | 39.568 | 11.184 | −1.687 | 1.00 | 46.38 | C |
| ATOM | 1956 | C | GLY | A | 122 | 40.066 | 10.056 | −2.530 | 1.00 | 46.66 | C |
| ATOM | 1957 | O | GLY | A | 122 | 39.309 | 9.308 | −3.132 | 1.00 | 44.50 | O |
| ATOM | 1958 | N | ALA | A | 123 | 41.369 | 9.924 | −2.491 | 1.00 | 47.30 | N |
| ATOM | 1960 | CA | ALA | A | 123 | 42.029 | 8.889 | −3.172 | 1.00 | 47.38 | C |
| ATOM | 1962 | CB | ALA | A | 123 | 43.273 | 9.481 | −3.863 | 1.00 | 49.98 | C |
| ATOM | 1966 | C | ALA | A | 123 | 42.350 | 7.675 | −2.244 | 1.00 | 44.62 | C |
| ATOM | 1967 | O | ALA | A | 123 | 41.818 | 7.512 | −1.158 | 1.00 | 43.85 | O |
| ATOM | 1968 | N | VAL | A | 124 | 43.114 | 6.733 | −2.791 | 1.00 | 45.41 | N |
| ATOM | 1970 | CA | VAL | A | 124 | 43.423 | 5.507 | −2.138 | 1.00 | 43.66 | C |
| ATOM | 1972 | CB | VAL | A | 124 | 42.721 | 4.412 | −2.827 | 1.00 | 42.78 | C |
| ATOM | 1974 | CG1 | VAL | A | 124 | 43.053 | 3.093 | −2.159 | 1.00 | 44.72 | C |
| ATOM | 1978 | CG2 | VAL | A | 124 | 41.231 | 4.593 | −2.655 | 1.00 | 42.04 | C |
| ATOM | 1982 | C | VAL | A | 124 | 44.902 | 5.256 | −2.142 | 1.00 | 44.48 | C |
| ATOM | 1983 | O | VAL | A | 124 | 45.611 | 5.612 | −3.083 | 1.00 | 46.68 | O |
| ATOM | 1984 | N | ARG | A | 125 | 45.391 | 4.622 | −1.099 | 1.00 | 43.14 | N |
| ATOM | 1986 | CA | ARG | A | 125 | 46.761 | 4.216 | −1.137 | 1.00 | 44.94 | C |
| ATOM | 1988 | CB | ARG | A | 125 | 47.589 | 5.115 | −0.277 | 1.00 | 47.27 | C |
| ATOM | 1991 | CG | ARG | A | 125 | 49.029 | 4.742 | −0.264 | 1.00 | 51.74 | C |
| ATOM | 1994 | CD | ARG | A | 125 | 49.897 | 5.906 | 0.139 | 1.00 | 57.79 | C |
| ATOM | 1997 | NE | ARG | A | 125 | 51.268 | 5.694 | −0.336 | 1.00 | 64.37 | N |
| ATOM | 1999 | CZ | ARG | A | 125 | 52.378 | 5.749 | 0.419 | 1.00 | 67.82 | C |
| ATOM | 2000 | NH1 | ARG | A | 125 | 52.312 | 6.066 | 1.746 | 1.00 | 67.17 | N |
| ATOM | 2003 | NH2 | ARG | A | 125 | 53.565 | 5.492 | −0.178 | 1.00 | 68.87 | N |
| ATOM | 2006 | C | ARG | A | 125 | 46.936 | 2.808 | −0.655 | 1.00 | 43.08 | C |
| ATOM | 2007 | O | ARG | A | 125 | 46.596 | 2.460 | 0.469 | 1.00 | 39.00 | O |
| ATOM | 2008 | N | PHE | A | 126 | 47.508 | 2.011 | −1.552 | 1.00 | 44.38 | N |
| ATOM | 2010 | CA | PHE | A | 126 | 48.137 | 0.783 | −1.167 | 1.00 | 44.03 | C |
| ATOM | 2012 | CB | PHE | A | 126 | 47.510 | −0.344 | −1.926 | 1.00 | 43.71 | C |
| ATOM | 2015 | CG | PHE | A | 126 | 46.218 | −0.807 | −1.391 | 1.00 | 40.01 | C |
| ATOM | 2016 | CD1 | PHE | A | 126 | 45.108 | −0.080 | −1.549 | 1.00 | 40.92 | C |
| ATOM | 2018 | CE1 | PHE | A | 126 | 43.887 | −0.550 | −1.063 | 1.00 | 39.27 | C |
| ATOM | 2020 | CZ | PHE | A | 126 | 43.775 | −1.763 | −0.456 | 1.00 | 38.94 | C |
| ATOM | 2022 | CE2 | PHE | A | 126 | 44.833 | −2.509 | −0.371 | 1.00 | 42.57 | C |
| ATOM | 2024 | CD2 | PHE | A | 126 | 46.088 | −2.034 | −0.854 | 1.00 | 42.75 | C |
| ATOM | 2026 | C | PHE | A | 126 | 49.648 | 0.852 | −1.465 | 1.00 | 46.82 | C |
| ATOM | 2027 | O | PHE | A | 126 | 50.036 | 1.090 | −2.614 | 1.00 | 47.64 | O |
| ATOM | 2028 | N | SER | A | 127 | 50.476 | 0.631 | −0.423 | 1.00 | 47.70 | N |
| ATOM | 2030 | CA | SER | A | 127 | 51.910 | 0.385 | −0.566 | 1.00 | 51.19 | C |
| ATOM | 2032 | CB | SER | A | 127 | 52.649 | 1.696 | −0.466 | 1.00 | 54.02 | C |
| ATOM | 2035 | OG | SER | A | 127 | 53.034 | 1.883 | 0.885 | 1.00 | 57.27 | O |
| ATOM | 2037 | C | SER | A | 127 | 52.481 | −0.594 | 0.485 | 1.00 | 51.56 | C |
| ATOM | 2038 | O | SER | A | 127 | 52.047 | −0.628 | 1.604 | 1.00 | 49.36 | O |
| ATOM | 2039 | N | ASN | A | 128 | 53.468 | −1.391 | 0.078 | 1.00 | 54.77 | N |
| ATOM | 2041 | CA | ASN | A | 128 | 54.264 | −2.229 | 0.968 | 1.00 | 56.21 | C |
| ATOM | 2043 | CB | ASN | A | 128 | 55.137 | −1.373 | 1.877 | 1.00 | 58.23 | C |
| ATOM | 2046 | CG | ASN | A | 128 | 56.046 | −0.407 | 1.097 | 1.00 | 62.17 | C |
| ATOM | 2047 | OD1 | ASN | A | 128 | 57.182 | −0.758 | 0.790 | 1.00 | 66.14 | O |
| ATOM | 2048 | ND2 | ASN | A | 128 | 55.556 | 0.817 | 0.799 | 1.00 | 58.01 | N |
| ATOM | 2051 | C | ASN | A | 128 | 53.402 | −3.228 | 1.755 | 1.00 | 54.35 | C |
| ATOM | 2052 | O | ASN | A | 128 | 53.461 | −3.340 | 2.990 | 1.00 | 53.42 | O |
| ATOM | 2053 | N | ASN | A | 129 | 52.624 | −3.990 | 0.999 | 1.00 | 53.03 | N |
| ATOM | 2055 | CA | ASN | A | 129 | 51.809 | −5.034 | 1.562 | 1.00 | 51.72 | C |
| ATOM | 2057 | CB | ASN | A | 129 | 50.355 | −4.712 | 1.281 | 1.00 | 48.91 | C |
| ATOM | 2060 | CG | ASN | A | 129 | 49.950 | −3.386 | 1.877 | 1.00 | 48.71 | C |
| ATOM | 2061 | OD1 | ASN | A | 129 | 49.956 | −3.219 | 3.097 | 1.00 | 46.84 | O |
| ATOM | 2062 | ND2 | ASN | A | 129 | 49.620 | −2.425 | 1.021 | 1.00 | 49.70 | N |
| ATOM | 2065 | C | ASN | A | 129 | 52.142 | −6.394 | 0.988 | 1.00 | 53.27 | C |
| ATOM | 2066 | O | ASN | A | 129 | 51.301 | −6.990 | 0.376 | 1.00 | 52.88 | O |
| ATOM | 2067 | N | PRO | A | 130 | 53.322 | −6.931 | 1.248 | 1.00 | 55.86 | N |
| ATOM | 2068 | CA | PRO | A | 130 | 53.849 | −8.030 | 0.441 | 1.00 | 58.30 | C |
| ATOM | 2070 | CB | PRO | A | 130 | 55.334 | −8.067 | 0.806 | 1.00 | 61.57 | C |
| ATOM | 2073 | CG | PRO | A | 130 | 55.422 | −7.439 | 2.170 | 1.00 | 61.58 | C |
| ATOM | 2076 | CD | PRO | A | 130 | 54.194 | −6.614 | 2.386 | 1.00 | 57.72 | C |
| ATOM | 2079 | C | PRO | A | 130 | 53.194 | −9.378 | 0.718 | 1.00 | 58.29 | C |
| ATOM | 2080 | O | PRO | A | 130 | 53.617 | −10.382 | 0.197 | 1.00 | 60.97 | O |
| ATOM | 2081 | N | ALA | A | 131 | 52.151 | −9.438 | 1.513 | 1.00 | 55.73 | N |
| ATOM | 2083 | CA | ALA | A | 131 | 51.452 | −10.692 | 1.621 | 1.00 | 54.80 | C |
| ATOM | 2085 | CB | ALA | A | 131 | 51.407 | −11.073 | 3.006 | 1.00 | 54.50 | C |
| ATOM | 2089 | C | ALA | A | 131 | 50.056 | −10.559 | 1.046 | 1.00 | 52.04 | C |
| ATOM | 2090 | O | ALA | A | 131 | 49.305 | −11.499 | 1.010 | 1.00 | 52.22 | O |
| ATOM | 2091 | N | LEU | A | 132 | 49.731 | −9.399 | 0.527 | 1.00 | 50.22 | N |
| ATOM | 2093 | CA | LEU | A | 132 | 48.347 | −9.100 | 0.235 | 1.00 | 48.25 | C |
| ATOM | 2095 | CB | LEU | A | 132 | 48.111 | −7.577 | 0.094 | 1.00 | 46.58 | C |
| ATOM | 2098 | CG | LEU | A | 132 | 46.693 | −7.049 | −0.140 | 1.00 | 42.72 | C |
| ATOM | 2100 | CD1 | LEU | A | 132 | 45.792 | −7.428 | 0.981 | 1.00 | 41.93 | C |
| ATOM | 2104 | CD2 | LEU | A | 132 | 46.701 | −5.522 | −0.330 | 1.00 | 41.41 | C |
| ATOM | 2108 | C | LEU | A | 132 | 47.923 | −9.826 | −1.003 | 1.00 | 49.53 | C |
| ATOM | 2109 | O | LEU | A | 132 | 48.735 | −10.093 | −1.883 | 1.00 | 51.28 | O |
| ATOM | 2110 | N | CYS | A | 133 | 46.636 | −10.148 | −1.031 | 1.00 | 49.32 | N |
| ATOM | 2112 | CA | CYS | A | 133 | 46.017 | −10.944 | −2.085 | 1.00 | 51.07 | C |
| ATOM | 2114 | CB | CYS | A | 133 | 45.605 | −12.286 | −1.533 | 1.00 | 51.93 | C |
| ATOM | 2117 | SG | CYS | A | 133 | 47.113 | −13.311 | −1.525 | 1.00 | 58.93 | S |
| ATOM | 2118 | C | CYS | A | 133 | 44.814 | −10.287 | −2.732 | 1.00 | 49.03 | C |
| ATOM | 2119 | O | CYS | A | 133 | 44.089 | −9.538 | −2.070 | 1.00 | 47.78 | O |
| ATOM | 2120 | N | ASN | A | 134 | 44.660 | −10.569 | −4.026 | 1.00 | 48.89 | N |
| ATOM | 2122 | CA | ASN | A | 134 | 43.496 | −10.312 | −4.800 | 1.00 | 47.55 | C |
| ATOM | 2124 | CB | ASN | A | 134 | 42.304 | −11.082 | −4.237 | 1.00 | 46.96 | C |
| ATOM | 2127 | CG | ASN | A | 134 | 42.563 | −12.593 | −4.084 | 1.00 | 49.52 | C |
| ATOM | 2128 | OD1 | ASN | A | 134 | 42.816 | −13.265 | −5.038 | 1.00 | 51.07 | O |
| ATOM | 2129 | ND2 | ASN | A | 134 | 42.457 | −13.112 | −2.870 | 1.00 | 48.52 | N |
| ATOM | 2132 | C | ASN | A | 134 | 43.169 | −8.832 | −5.039 | 1.00 | 45.92 | C |
| ATOM | 2133 | O | ASN | A | 134 | 42.597 | −8.494 | −6.061 | 1.00 | 45.65 | O |
| ATOM | 2134 | N | VAL | A | 135 | 43.530 | −7.933 | −4.135 | 1.00 | 45.56 | N |
| ATOM | 2136 | CA | VAL | A | 135 | 42.985 | −6.552 | −4.156 | 1.00 | 43.82 | C |
| ATOM | 2138 | CB | VAL | A | 135 | 43.400 | −5.780 | −2.932 | 1.00 | 42.47 | C |
| ATOM | 2140 | CG1 | VAL | A | 135 | 43.037 | −4.266 | −3.017 | 1.00 | 41.77 | C |
| ATOM | 2144 | CG2 | VAL | A | 135 | 42.786 | −6.384 | −1.737 | 1.00 | 41.35 | C |
| ATOM | 2148 | C | VAL | A | 135 | 43.493 | −5.880 | −5.431 | 1.00 | 45.40 | C |
| ATOM | 2149 | O | VAL | A | 135 | 42.723 | −5.205 | −6.070 | 1.00 | 45.40 | O |
| ATOM | 2150 | N | GLU | A | 136 | 44.755 | −6.158 | −5.796 | 1.00 | 46.87 | N |
| ATOM | 2152 | CA | GLU | A | 136 | 45.404 | −5.750 | −7.035 | 1.00 | 49.44 | C |
| ATOM | 2154 | CB | GLU | A | 136 | 46.677 | −6.586 | −7.316 | 1.00 | 52.90 | C |
| ATOM | 2157 | CG | GLU | A | 136 | 47.654 | −6.085 | −8.413 | 1.00 | 57.64 | C |
| ATOM | 2160 | CD | GLU | A | 136 | 48.784 | −7.097 | −8.714 | 1.00 | 66.03 | C |
| ATOM | 2161 | OE1 | GLU | A | 136 | 49.015 | −7.981 | −7.798 | 1.00 | 68.30 | O |
| ATOM | 2162 | OE2 | GLU | A | 136 | 49.415 | −7.041 | −9.837 | 1.00 | 70.61 | O |
| ATOM | 2163 | C | GLU | A | 136 | 44.526 | −5.895 | −8.238 | 1.00 | 50.26 | C |
| ATOM | 2164 | O | GLU | A | 136 | 44.781 | −5.226 | −9.234 | 1.00 | 52.27 | O |
| ATOM | 2165 | N | SER | A | 137 | 43.516 | −6.745 | −8.189 | 1.00 | 49.34 | N |
| ATOM | 2167 | CA | SER | A | 137 | 42.673 | −6.934 | −9.365 | 1.00 | 51.02 | C |
| ATOM | 2169 | CB | SER | A | 137 | 42.120 | −8.342 | −9.390 | 1.00 | 51.77 | C |
| ATOM | 2172 | OG | SER | A | 137 | 41.372 | −8.537 | −8.213 | 1.00 | 49.79 | O |
| ATOM | 2174 | C | SER | A | 137 | 41.521 | −5.953 | −9.408 | 1.00 | 49.35 | C |
| ATOM | 2175 | O | SER | A | 137 | 40.737 | −5.909 | −10.335 | 1.00 | 50.09 | O |
| ATOM | 2176 | N | ILE | A | 138 | 41.427 | −5.128 | −8.420 | 1.00 | 47.68 | N |
| ATOM | 2178 | CA | ILE | A | 138 | 40.207 | −4.429 | −8.272 | 1.00 | 47.46 | C |
| ATOM | 2180 | CB | ILE | A | 138 | 39.947 | −4.236 | −6.830 | 1.00 | 45.92 | C |
| ATOM | 2182 | CG1 | ILE | A | 138 | 39.161 | −5.479 | −6.402 | 1.00 | 50.04 | C |
| ATOM | 2185 | CD1 | ILE | A | 138 | 38.907 | −5.482 | −4.979 | 1.00 | 52.31 | C |
| ATOM | 2189 | CG2 | ILE | A | 138 | 39.130 | −3.036 | −6.535 | 1.00 | 44.29 | C |
| ATOM | 2193 | C | ILE | A | 138 | 40.270 | −3.168 | −9.014 | 1.00 | 48.13 | C |
| ATOM | 2194 | O | ILE | A | 138 | 41.305 | −2.535 | −9.060 | 1.00 | 49.23 | O |
| ATOM | 2195 | N | GLN | A | 139 | 39.110 | −2.834 | −9.567 | 1.00 | 48.49 | N |
| ATOM | 2197 | CA | GLN | A | 139 | 38.826 | −1.708 | −10.439 | 1.00 | 48.81 | C |
| ATOM | 2199 | CB | GLN | A | 139 | 37.724 | −2.104 | −11.446 | 1.00 | 50.08 | C |
| ATOM | 2202 | CG | GLN | A | 139 | 38.234 | −3.051 | −12.588 | 1.00 | 52.30 | C |
| ATOM | 2205 | CD | GLN | A | 139 | 37.175 | −3.535 | −13.588 | 1.00 | 54.29 | C |
| ATOM | 2206 | OE1 | GLN | A | 139 | 36.906 | −2.864 | −14.560 | 1.00 | 59.63 | O |
| ATOM | 2207 | NE2 | GLN | A | 139 | 36.617 | −4.708 | −13.361 | 1.00 | 52.60 | N |
| ATOM | 2210 | C | GLN | A | 139 | 38.326 | −0.553 | −9.603 | 1.00 | 48.02 | C |
| ATOM | 2211 | O | GLN | A | 139 | 37.130 | −0.409 | −9.361 | 1.00 | 48.86 | O |
| ATOM | 2212 | N | TRP | A | 140 | 39.238 | 0.279 | −9.138 | 1.00 | 47.32 | N |
| ATOM | 2214 | CA | TRP | A | 140 | 38.876 | 1.416 | −8.309 | 1.00 | 45.95 | C |
| ATOM | 2216 | CB | TRP | A | 140 | 40.180 | 2.000 | −7.785 | 1.00 | 46.14 | C |
| ATOM | 2219 | CG | TRP | A | 140 | 40.885 | 0.963 | −6.994 | 1.00 | 46.00 | C |
| ATOM | 2220 | CD1 | TRP | A | 140 | 41.802 | 0.118 | −7.451 | 1.00 | 49.63 | C |
| ATOM | 2222 | NE1 | TRP | A | 140 | 42.183 | −0.754 | −6.450 | 1.00 | 52.24 | N |
| ATOM | 2224 | CE2 | TRP | A | 140 | 41.481 | −0.469 | −5.318 | 1.00 | 45.13 | C |
| ATOM | 2225 | CD2 | TRP | A | 140 | 40.644 | 0.601 | −5.619 | 1.00 | 46.06 | C |
| ATOM | 2226 | CE3 | TRP | A | 140 | 39.843 | 1.122 | −4.603 | 1.00 | 47.07 | C |
| ATOM | 2228 | CZ3 | TRP | A | 140 | 39.902 | 0.531 | −3.331 | 1.00 | 46.32 | C |
| ATOM | 2230 | CH2 | TRP | A | 140 | 40.751 | −0.540 | −3.099 | 1.00 | 44.90 | C |
| ATOM | 2232 | CZ2 | TRP | A | 140 | 41.559 | −1.032 | −4.085 | 1.00 | 44.06 | C |
| ATOM | 2234 | C | TRP | A | 140 | 37.958 | 2.468 | −8.988 | 1.00 | 46.64 | C |
| ATOM | 2235 | O | TRP | A | 140 | 37.156 | 3.152 | −8.417 | 1.00 | 47.16 | O |
| ATOM | 2236 | N | ARG | A | 141 | 38.039 | 2.549 | −10.251 | 1.00 | 49.56 | N |
| ATOM | 2238 | CA | ARG | A | 141 | 37.281 | 3.487 | −11.006 | 1.00 | 51.70 | C |
| ATOM | 2240 | CB | ARG | A | 141 | 37.690 | 3.287 | −12.479 | 1.00 | 54.96 | C |
| ATOM | 2243 | CG | ARG | A | 141 | 37.314 | 4.259 | −13.453 | 1.00 | 59.89 | C |
| ATOM | 2246 | CD | ARG | A | 141 | 37.332 | 3.683 | −14.883 | 1.00 | 68.80 | C |
| ATOM | 2249 | NE | ARG | A | 141 | 36.925 | 4.702 | −15.900 | 1.00 | 76.88 | N |
| ATOM | 2251 | CZ | ARG | A | 141 | 36.168 | 4.420 | −16.981 | 1.00 | 79.58 | C |
| ATOM | 2252 | NH1 | ARG | A | 141 | 35.680 | 3.165 | −17.156 | 1.00 | 77.54 | N |
| ATOM | 2255 | NH2 | ARG | A | 141 | 35.910 | 5.388 | −17.889 | 1.00 | 83.87 | N |
| ATOM | 2258 | C | ARG | A | 141 | 35.805 | 3.315 | −10.714 | 1.00 | 51.04 | C |
| ATOM | 2259 | O | ARG | A | 141 | 35.077 | 4.284 | −10.763 | 1.00 | 52.62 | O |
| ATOM | 2260 | N | ASP | A | 142 | 35.337 | 2.129 | −10.354 | 1.00 | 50.38 | N |
| ATOM | 2262 | CA | ASP | A | 142 | 33.936 | 2.014 | −9.928 | 1.00 | 50.96 | C |
| ATOM | 2264 | CB | ASP | A | 142 | 33.502 | 0.590 | −9.927 | 1.00 | 51.24 | C |
| ATOM | 2267 | CG | ASP | A | 142 | 32.027 | 0.437 | −9.613 | 1.00 | 56.27 | C |
| ATOM | 2268 | OD1 | ASP | A | 142 | 31.590 | 0.872 | −8.537 | 1.00 | 64.27 | O |
| ATOM | 2269 | OD2 | ASP | A | 142 | 31.195 | −0.113 | −10.333 | 1.00 | 62.04 | O |
| ATOM | 2270 | C | ASP | A | 142 | 33.730 | 2.575 | −8.491 | 1.00 | 48.89 | C |
| ATOM | 2271 | O | ASP | A | 142 | 32.639 | 3.015 | −8.113 | 1.00 | 47.96 | O |
| ATOM | 2272 | N | ILE | A | 143 | 34.781 | 2.510 | −7.685 | 1.00 | 47.20 | N |
| ATOM | 2274 | CA | ILE | A | 143 | 34.745 | 2.956 | −6.294 | 1.00 | 44.55 | C |
| ATOM | 2276 | CB | ILE | A | 143 | 35.712 | 2.142 | −5.533 | 1.00 | 42.17 | C |
| ATOM | 2278 | CG1 | ILE | A | 143 | 35.086 | 0.768 | −5.327 | 1.00 | 40.78 | C |
| ATOM | 2281 | CD1 | ILE | A | 143 | 36.133 | −0.301 | −5.046 | 1.00 | 40.44 | C |
| ATOM | 2285 | CG2 | ILE | A | 143 | 36.018 | 2.787 | −4.247 | 1.00 | 41.34 | C |
| ATOM | 2289 | C | ILE | A | 143 | 35.044 | 4.401 | −6.069 | 1.00 | 44.56 | C |
| ATOM | 2290 | O | ILE | A | 143 | 34.327 | 5.095 | −5.380 | 1.00 | 44.62 | O |
| ATOM | 2291 | N | VAL | A | 144 | 36.124 | 4.863 | −6.630 | 1.00 | 45.00 | N |
| ATOM | 2293 | CA | VAL | A | 144 | 36.509 | 6.218 | −6.403 | 1.00 | 45.50 | C |
| ATOM | 2295 | CB | VAL | A | 144 | 38.028 | 6.287 | −6.507 | 1.00 | 45.97 | C |
| ATOM | 2297 | CG1 | VAL | A | 144 | 38.574 | 7.708 | −6.491 | 1.00 | 48.38 | C |
| ATOM | 2301 | CG2 | VAL | A | 144 | 38.596 | 5.619 | −5.328 | 1.00 | 43.75 | C |
| ATOM | 2305 | C | VAL | A | 144 | 35.797 | 7.249 | −7.293 | 1.00 | 48.79 | C |
| ATOM | 2306 | O | VAL | A | 144 | 35.338 | 7.047 | −8.422 | 1.00 | 49.84 | O |
| ATOM | 2307 | N | SER | A | 145 | 35.723 | 8.445 | −6.763 | 1.00 | 51.81 | N |
| ATOM | 2309 | CA | SER | A | 145 | 35.288 | 9.568 | −7.616 | 1.00 | 54.78 | C |
| ATOM | 2311 | CB | SER | A | 145 | 34.926 | 10.760 | −6.750 | 1.00 | 54.47 | C |
| ATOM | 2314 | OG | SER | A | 145 | 34.427 | 11.741 | −7.626 | 1.00 | 59.24 | O |
| ATOM | 2316 | C | SER | A | 145 | 36.287 | 9.966 | −8.735 | 1.00 | 56.23 | C |
| ATOM | 2317 | O | SER | A | 145 | 37.469 | 10.176 | −8.515 | 1.00 | 55.05 | O |
| ATOM | 2318 | N | SER | A | 146 | 35.814 | 10.152 | −9.935 | 1.00 | 58.67 | N |
| ATOM | 2320 | CA | SER | A | 146 | 36.764 | 10.567 | −10.927 | 1.00 | 61.98 | C |
| ATOM | 2322 | CB | SER | A | 146 | 36.061 | 10.701 | −12.244 | 1.00 | 65.22 | C |
| ATOM | 2325 | OG | SER | A | 146 | 35.229 | 11.815 | −12.148 | 1.00 | 69.73 | O |
| ATOM | 2327 | C | SER | A | 146 | 37.586 | 11.851 | −10.591 | 1.00 | 63.73 | C |
| ATOM | 2328 | O | SER | A | 146 | 38.718 | 12.001 | −11.061 | 1.00 | 66.06 | O |
| ATOM | 2329 | N | ASP | A | 147 | 37.110 | 12.782 | −9.776 | 1.00 | 63.35 | N |
| ATOM | 2331 | CA | ASP | A | 147 | 38.047 | 13.855 | −9.448 | 1.00 | 64.51 | C |
| ATOM | 2333 | CB | ASP | A | 147 | 37.388 | 14.938 | −8.643 | 1.00 | 65.88 | C |
| ATOM | 2336 | CG | ASP | A | 147 | 36.191 | 15.484 | −9.315 | 1.00 | 70.56 | C |
| ATOM | 2337 | OD1 | ASP | A | 147 | 36.324 | 15.944 | −10.449 | 1.00 | 76.86 | O |
| ATOM | 2338 | OD2 | ASP | A | 147 | 35.062 | 15.475 | −8.799 | 1.00 | 74.60 | O |
| ATOM | 2339 | C | ASP | A | 147 | 39.320 | 13.451 | −8.694 | 1.00 | 61.87 | C |
| ATOM | 2340 | O | ASP | A | 147 | 40.181 | 14.271 | −8.574 | 1.00 | 64.00 | O |
| ATOM | 2341 | N | PHE | A | 148 | 39.443 | 12.235 | −8.160 | 1.00 | 58.00 | N |
| ATOM | 2343 | CA | PHE | A | 148 | 40.589 | 11.831 | −7.323 | 1.00 | 55.65 | C |
| ATOM | 2345 | CB | PHE | A | 148 | 40.100 | 11.392 | −5.912 | 1.00 | 52.67 | C |
| ATOM | 2348 | CG | PHE | A | 148 | 39.418 | 12.463 | −5.124 | 1.00 | 52.51 | C |
| ATOM | 2349 | CD1 | PHE | A | 148 | 38.059 | 12.760 | −5.313 | 1.00 | 55.87 | C |
| ATOM | 2351 | CE1 | PHE | A | 148 | 37.408 | 13.812 | −4.627 | 1.00 | 53.04 | C |
| ATOM | 2353 | CZ | PHE | A | 148 | 38.100 | 14.471 | −3.764 | 1.00 | 53.77 | C |
| ATOM | 2355 | CE2 | PHE | A | 148 | 39.466 | 14.202 | −3.553 | 1.00 | 50.27 | C |
| ATOM | 2357 | CD2 | PHE | A | 148 | 40.105 | 13.203 | −4.221 | 1.00 | 53.15 | C |
| ATOM | 2359 | C | PHE | A | 148 | 41.411 | 10.674 | −7.958 | 1.00 | 55.84 | C |
| ATOM | 2360 | O | PHE | A | 148 | 42.390 | 10.172 | −7.375 | 1.00 | 54.76 | O |
| ATOM | 2361 | N | LEU | A | 149 | 40.992 | 10.208 | −9.141 | 1.00 | 57.46 | N |
| ATOM | 2363 | CA | LEU | A | 149 | 41.756 | 9.207 | −9.902 | 1.00 | 57.20 | C |
| ATOM | 2365 | CB | LEU | A | 149 | 41.124 | 8.951 | −11.285 | 1.00 | 58.59 | C |
| ATOM | 2368 | CG | LEU | A | 149 | 39.781 | 8.163 | −11.362 | 1.00 | 57.42 | C |
| ATOM | 2370 | CD1 | LEU | A | 149 | 39.542 | 7.556 | −12.713 | 1.00 | 59.65 | C |
| ATOM | 2374 | CD2 | LEU | A | 149 | 39.590 | 7.015 | −10.313 | 1.00 | 54.35 | C |
| ATOM | 2378 | C | LEU | A | 149 | 43.154 | 9.743 | −10.028 | 1.00 | 59.44 | C |
| ATOM | 2379 | O | LEU | A | 149 | 44.122 | 9.110 | −9.706 | 1.00 | 57.88 | O |
| ATOM | 2380 | N | SER | A | 150 | 43.237 | 10.997 | −10.427 | 1.00 | 63.31 | N |
| ATOM | 2382 | CA | SER | A | 150 | 44.517 | 11.658 | −10.439 | 1.00 | 65.95 | C |
| ATOM | 2384 | CB | SER | A | 150 | 44.345 | 13.107 | −10.848 | 1.00 | 69.04 | C |
| ATOM | 2387 | OG | SER | A | 150 | 44.468 | 13.155 | −12.260 | 1.00 | 74.02 | O |
| ATOM | 2389 | C | SER | A | 150 | 45.359 | 11.553 | −9.165 | 1.00 | 64.15 | C |
| ATOM | 2390 | O | SER | A | 150 | 46.550 | 11.630 | −9.276 | 1.00 | 66.94 | O |
| ATOM | 2391 | N | ASN | A | 151 | 44.802 | 11.413 | −7.962 | 1.00 | 60.55 | N |
| ATOM | 2393 | CA | ASN | A | 151 | 45.676 | 11.373 | −6.785 | 1.00 | 59.05 | C |
| ATOM | 2395 | CB | ASN | A | 151 | 45.280 | 12.526 | −5.824 | 1.00 | 58.72 | C |
| ATOM | 2398 | CG | ASN | A | 151 | 45.979 | 13.869 | −6.202 | 1.00 | 65.21 | C |
| ATOM | 2399 | OD1 | ASN | A | 151 | 45.616 | 14.591 | −7.128 | 1.00 | 66.28 | O |
| ATOM | 2400 | ND2 | ASN | A | 151 | 47.026 | 14.157 | −5.506 | 1.00 | 72.90 | N |
| ATOM | 2403 | C | ASN | A | 151 | 45.879 | 9.943 | −6.181 | 1.00 | 55.44 | C |
| ATOM | 2404 | O | ASN | A | 151 | 46.471 | 9.774 | −5.128 | 1.00 | 53.43 | O |
| ATOM | 2405 | N | MET | A | 152 | 45.499 | 8.901 | −6.932 | 1.00 | 54.65 | N |
| ATOM | 2407 | CA | MET | A | 152 | 45.653 | 7.496 | −6.467 | 1.00 | 52.66 | C |
| ATOM | 2409 | CB | MET | A | 152 | 44.910 | 6.533 | −7.366 | 1.00 | 51.86 | C |
| ATOM | 2412 | CG | MET | A | 152 | 43.443 | 6.813 | −7.552 | 1.00 | 52.03 | C |
| ATOM | 2415 | SD | MET | A | 152 | 42.338 | 6.049 | −6.388 | 1.00 | 50.06 | S |
| ATOM | 2416 | CE | MET | A | 152 | 42.743 | 4.273 | −6.557 | 1.00 | 48.82 | C |
| ATOM | 2420 | C | MET | A | 152 | 47.124 | 7.024 | −6.371 | 1.00 | 54.06 | C |
| ATOM | 2421 | O | MET | A | 152 | 47.951 | 7.492 | −7.134 | 1.00 | 56.68 | O |
| ATOM | 2422 | N | SER | A | 153 | 47.449 | 6.144 | −5.399 | 1.00 | 52.56 | N |
| ATOM | 2424 | CA | SER | A | 153 | 48.828 | 5.589 | −5.246 | 1.00 | 54.12 | C |
| ATOM | 2426 | CB | SER | A | 153 | 49.601 | 6.341 | −4.187 | 1.00 | 54.43 | C |
| ATOM | 2429 | OG | SER | A | 153 | 50.968 | 6.247 | −4.512 | 1.00 | 56.90 | O |
| ATOM | 2431 | C | SER | A | 153 | 48.953 | 4.069 | −4.949 | 1.00 | 52.77 | C |
| ATOM | 2432 | O | SER | A | 153 | 49.268 | 3.659 | −3.817 | 1.00 | 52.35 | O |
| ATOM | 2433 | N | MET | A | 154 | 48.749 | 3.276 | −5.995 | 1.00 | 52.33 | N |
| ATOM | 2435 | CA | MET | A | 154 | 48.550 | 1.862 | −5.893 | 1.00 | 50.75 | C |
| ATOM | 2437 | CB | MET | A | 154 | 47.455 | 1.440 | −6.811 | 1.00 | 49.97 | C |
| ATOM | 2440 | CG | MET | A | 154 | 46.216 | 2.318 | −6.656 | 1.00 | 50.89 | C |
| ATOM | 2443 | SD | MET | A | 154 | 45.282 | 1.929 | −5.148 | 1.00 | 58.01 | S |
| ATOM | 2444 | CE | MET | A | 154 | 45.749 | 3.387 | −4.448 | 1.00 | 57.15 | C |
| ATOM | 2448 | C | MET | A | 154 | 49.802 | 1.157 | −6.255 | 1.00 | 52.92 | C |
| ATOM | 2449 | O | MET | A | 154 | 50.276 | 1.278 | −7.330 | 1.00 | 54.18 | O |
| ATOM | 2450 | N | ASP | A | 155 | 50.346 | 0.414 | −5.315 | 1.00 | 53.59 | N |
| ATOM | 2452 | CA | ASP | A | 155 | 51.559 | −0.296 | −5.564 | 1.00 | 57.73 | C |
| ATOM | 2454 | CB | ASP | A | 155 | 52.786 | 0.526 | −5.195 | 1.00 | 60.70 | C |
| ATOM | 2457 | CG | ASP | A | 155 | 54.088 | −0.300 | −5.273 | 1.00 | 67.99 | C |
| ATOM | 2458 | OD1 | ASP | A | 155 | 54.469 | −0.830 | −6.389 | 1.00 | 70.11 | O |
| ATOM | 2459 | OD2 | ASP | A | 155 | 54.773 | −0.487 | −4.215 | 1.00 | 74.44 | O |
| ATOM | 2460 | C | ASP | A | 155 | 51.489 | −1.670 | −4.882 | 1.00 | 56.85 | C |
| ATOM | 2461 | O | ASP | A | 155 | 51.675 | −1.845 | −3.662 | 1.00 | 55.16 | O |
| ATOM | 2462 | N | PHE | A | 156 | 51.232 | −2.651 | −5.733 | 1.00 | 57.62 | N |
| ATOM | 2464 | CA | PHE | A | 156 | 50.904 | −3.965 | −5.298 | 1.00 | 57.25 | C |
| ATOM | 2466 | CB | PHE | A | 156 | 49.701 | −4.410 | −6.080 | 1.00 | 55.63 | C |
| ATOM | 2469 | CG | PHE | A | 156 | 48.424 | −3.817 | −5.645 | 1.00 | 51.82 | C |
| ATOM | 2470 | CD1 | PHE | A | 156 | 47.824 | −2.852 | −6.408 | 1.00 | 48.57 | C |
| ATOM | 2472 | CE1 | PHE | A | 156 | 46.643 | −2.329 | −6.067 | 1.00 | 48.09 | C |
| ATOM | 2474 | CZ | PHE | A | 156 | 46.005 | −2.746 | −4.921 | 1.00 | 49.29 | C |
| ATOM | 2476 | CE2 | PHE | A | 156 | 46.573 | −3.759 | −4.128 | 1.00 | 49.85 | C |
| ATOM | 2478 | CD2 | PHE | A | 156 | 47.775 | −4.298 | −4.513 | 1.00 | 51.25 | C |
| ATOM | 2480 | C | PHE | A | 156 | 52.031 | −4.922 | −5.663 | 1.00 | 62.02 | C |
| ATOM | 2481 | O | PHE | A | 156 | 52.258 | −5.121 | −6.862 | 1.00 | 65.96 | O |
| ATOM | 2482 | N | GLN | A | 157 | 52.711 | −5.558 | −4.693 | 1.00 | 63.64 | N |
| ATOM | 2484 | CA | GLN | A | 157 | 53.730 | −6.585 | −5.019 | 1.00 | 67.83 | C |
| ATOM | 2486 | CB | GLN | A | 157 | 55.158 | −6.019 | −5.121 | 1.00 | 71.20 | C |
| ATOM | 2489 | CG | GLN | A | 157 | 55.232 | −4.741 | −5.955 | 1.00 | 74.76 | C |
| ATOM | 2492 | CD | GLN | A | 157 | 56.512 | −3.922 | −5.738 | 1.00 | 81.43 | C |
| ATOM | 2493 | OE1 | GLN | A | 157 | 57.596 | −4.501 | −5.524 | 1.00 | 84.93 | O |
| ATOM | 2494 | NE2 | GLN | A | 157 | 56.394 | −2.570 | −5.832 | 1.00 | 80.80 | N |
| ATOM | 2497 | C | GLN | A | 157 | 53.694 | −7.701 | −3.995 | 1.00 | 67.85 | C |
| ATOM | 2498 | O | GLN | A | 157 | 54.239 | −7.549 | −2.884 | 1.00 | 68.89 | O |
| ATOM | 2499 | N | ASN | A | 158 | 53.049 | −8.824 | −4.343 | 1.00 | 67.34 | N |
| ATOM | 2501 | CA | ASN | A | 158 | 52.985 | −9.931 | −3.387 | 1.00 | 66.37 | C |
| ATOM | 2503 | CB | ASN | A | 158 | 51.748 | −10.825 | −3.573 | 1.00 | 65.25 | C |
| ATOM | 2506 | CG | ASN | A | 158 | 51.555 | −11.805 | −2.408 | 1.00 | 65.80 | C |
| ATOM | 2507 | OD1 | ASN | A | 158 | 52.434 | −12.615 | −2.126 | 1.00 | 69.30 | O |
| ATOM | 2508 | ND2 | ASN | A | 158 | 50.396 | −11.749 | −1.760 | 1.00 | 62.84 | N |
| ATOM | 2511 | C | ASN | A | 158 | 54.252 | −10.683 | −3.607 | 1.00 | 68.88 | C |
| ATOM | 2512 | O | ASN | A | 158 | 54.482 | −11.075 | −4.714 | 1.00 | 71.15 | O |
| ATOM | 2513 | N | HIS | A | 159 | 55.079 | −10.768 | −2.562 | 1.00 | 68.92 | N |
| ATOM | 2515 | CA | HIS | A | 159 | 56.367 | −11.474 | −2.511 | 1.00 | 72.75 | C |
| ATOM | 2517 | CB | HIS | A | 159 | 57.529 | −10.454 | −2.416 | 1.00 | 74.34 | C |
| ATOM | 2525 | C | HIS | A | 159 | 56.430 | −12.503 | −1.321 | 1.00 | 73.55 | C |
| ATOM | 2526 | O | HIS | A | 159 | 57.483 | −13.106 | −1.060 | 1.00 | 77.25 | O |
| ATOM | 2527 | N | LEU | A | 160 | 55.312 | −12.726 | −0.620 | 1.00 | 70.71 | N |
| ATOM | 2529 | CA | LEU | A | 160 | 55.300 | −13.613 | 0.558 | 1.00 | 71.96 | C |
| ATOM | 2531 | CB | LEU | A | 160 | 54.955 | −12.858 | 1.835 | 1.00 | 69.16 | C |
| ATOM | 2534 | CG | LEU | A | 160 | 56.170 | −12.018 | 2.242 | 1.00 | 72.49 | C |
| ATOM | 2536 | CD1 | LEU | A | 160 | 55.879 | −11.038 | 3.417 | 1.00 | 70.29 | C |
| ATOM | 2540 | CD2 | LEU | A | 160 | 57.406 | −12.963 | 2.560 | 1.00 | 78.18 | C |
| ATOM | 2544 | C | LEU | A | 160 | 54.436 | −14.860 | 0.466 | 1.00 | 72.80 | C |
| ATOM | 2545 | O | LEU | A | 160 | 54.715 | −15.905 | 1.146 | 1.00 | 76.27 | O |
| ATOM | 2546 | N | GLY | A | 161 | 53.426 | −14.830 | −0.387 | 1.00 | 70.92 | N |
| ATOM | 2548 | CA | GLY | A | 161 | 52.682 | −16.052 | −0.588 | 1.00 | 71.53 | C |
| ATOM | 2551 | C | GLY | A | 161 | 52.337 | −16.324 | −2.010 | 1.00 | 72.10 | C |
| ATOM | 2552 | O | GLY | A | 161 | 52.925 | −15.725 | −2.936 | 1.00 | 73.91 | O |
| ATOM | 2553 | N | SER | A | 162 | 51.354 | −17.218 | −2.151 | 1.00 | 71.50 | N |
| ATOM | 2555 | CA | SER | A | 162 | 50.850 | −17.612 | −3.430 | 1.00 | 71.80 | C |
| ATOM | 2557 | CB | SER | A | 162 | 51.072 | −19.072 | −3.631 | 1.00 | 75.27 | C |
| ATOM | 2560 | OG | SER | A | 162 | 49.976 | −19.671 | −3.029 | 1.00 | 75.08 | O |
| ATOM | 2562 | C | SER | A | 162 | 49.382 | −17.287 | −3.697 | 1.00 | 68.40 | C |
| ATOM | 2563 | O | SER | A | 162 | 49.076 | −17.118 | −4.826 | 1.00 | 71.58 | O |
| ATOM | 2564 | N | CYS | A | 163 | 48.443 | −17.195 | −2.785 | 1.00 | 64.77 | N |
| ATOM | 2566 | CA | CYS | A | 163 | 47.190 | −16.558 | −3.248 | 1.00 | 62.32 | C |
| ATOM | 2568 | CB | CYS | A | 163 | 47.482 | −15.350 | −4.121 | 1.00 | 60.54 | C |
| ATOM | 2571 | SG | CYS | A | 163 | 48.195 | −14.039 | −3.164 | 1.00 | 65.07 | S |
| ATOM | 2572 | C | CYS | A | 163 | 46.203 | −17.327 | −4.092 | 1.00 | 62.09 | C |
| ATOM | 2573 | O | CYS | A | 163 | 46.486 | −17.842 | −5.125 | 1.00 | 64.63 | O |
| ATOM | 2574 | N | GLN | A | 164 | 44.966 | −17.252 | −3.712 | 1.00 | 59.87 | N |
| ATOM | 2576 | CA | GLN | A | 164 | 43.963 | −17.875 | −4.490 | 1.00 | 59.87 | C |
| ATOM | 2578 | CB | GLN | A | 164 | 42.775 | −18.151 | −3.577 | 1.00 | 58.78 | C |
| ATOM | 2581 | CG | GLN | A | 164 | 43.135 | −19.114 | −2.394 | 1.00 | 60.45 | C |
| ATOM | 2584 | CD | GLN | A | 164 | 42.289 | −18.848 | −1.173 | 1.00 | 58.32 | C |
| ATOM | 2585 | OE1 | GLN | A | 164 | 41.246 | −19.446 | −1.065 | 1.00 | 62.19 | O |
| ATOM | 2586 | NE2 | GLN | A | 164 | 42.706 | −17.902 | −0.272 | 1.00 | 60.77 | N |
| ATOM | 2589 | C | GLN | A | 164 | 43.615 | −17.003 | −5.717 | 1.00 | 58.68 | C |
| ATOM | 2590 | O | GLN | A | 164 | 44.054 | −15.875 | −5.910 | 1.00 | 55.31 | O |
| ATOM | 2591 | N | LYS | A | 165 | 42.840 | −17.597 | −6.587 | 1.00 | 60.41 | N |
| ATOM | 2593 | CA | LYS | A | 165 | 42.374 | −16.938 | −7.780 | 1.00 | 59.80 | C |
| ATOM | 2595 | CB | LYS | A | 165 | 42.587 | −17.888 | −8.966 | 1.00 | 63.00 | C |
| ATOM | 2598 | CG | LYS | A | 165 | 44.023 | −18.081 | −9.347 | 1.00 | 64.02 | C |
| ATOM | 2601 | CD | LYS | A | 165 | 44.046 | −18.676 | −10.685 | 1.00 | 70.24 | C |
| ATOM | 2604 | CE | LYS | A | 165 | 45.354 | −19.359 | −10.979 | 1.00 | 75.93 | C |
| ATOM | 2607 | NZ | LYS | A | 165 | 45.322 | −20.005 | −12.321 | 1.00 | 78.93 | N |
| ATOM | 2611 | C | LYS | A | 165 | 40.898 | −16.505 | −7.632 | 1.00 | 57.91 | C |
| ATOM | 2612 | O | LYS | A | 165 | 40.082 | −17.115 | −6.894 | 1.00 | 57.15 | O |
| ATOM | 2613 | N | CYS | A | 166 | 40.574 | −15.429 | −8.331 | 1.00 | 56.63 | N |
| ATOM | 2615 | CA | CYS | A | 166 | 39.229 | −14.949 | −8.421 | 1.00 | 56.14 | C |
| ATOM | 2617 | CB | CYS | A | 166 | 39.265 | −13.725 | −9.280 | 1.00 | 56.07 | C |
| ATOM | 2620 | SG | CYS | A | 166 | 39.966 | −12.314 | −8.384 | 1.00 | 60.17 | S |
| ATOM | 2621 | C | CYS | A | 166 | 38.278 | −15.928 | −9.059 | 1.00 | 58.20 | C |
| ATOM | 2622 | O | CYS | A | 166 | 38.612 | −16.593 | −9.993 | 1.00 | 60.87 | O |
| ATOM | 2623 | N | ASP | A | 167 | 37.054 | −15.995 | −8.604 | 1.00 | 58.01 | N |
| ATOM | 2625 | CA | ASP | A | 167 | 36.071 | −16.803 | −9.313 | 1.00 | 62.21 | C |
| ATOM | 2627 | CB | ASP | A | 167 | 34.731 | −16.757 | −8.577 | 1.00 | 61.75 | C |
| ATOM | 2630 | CG | ASP | A | 167 | 33.981 | −18.042 | −8.691 | 1.00 | 67.21 | C |
| ATOM | 2631 | OD1 | ASP | A | 167 | 33.252 | −18.146 | −9.682 | 1.00 | 71.66 | O |
| ATOM | 2632 | OD2 | ASP | A | 167 | 34.098 | −19.030 | −7.887 | 1.00 | 71.36 | O |
| ATOM | 2633 | C | ASP | A | 167 | 35.907 | −16.370 | −10.772 | 1.00 | 64.34 | C |
| ATOM | 2634 | O | ASP | A | 167 | 35.932 | −15.189 | −11.077 | 1.00 | 64.04 | O |
| ATOM | 2635 | N | PRO | A | 168 | 35.733 | −17.276 | −11.712 | 1.00 | 69.18 | N |
| ATOM | 2636 | CA | PRO | A | 168 | 35.529 | −16.843 | −13.117 | 1.00 | 71.67 | C |
| ATOM | 2638 | CB | PRO | A | 168 | 35.482 | −18.135 | −13.880 | 1.00 | 75.31 | C |
| ATOM | 2641 | CG | PRO | A | 168 | 34.937 | −19.094 | −12.867 | 1.00 | 75.75 | C |
| ATOM | 2644 | CD | PRO | A | 168 | 35.648 | −18.736 | −11.577 | 1.00 | 72.39 | C |
| ATOM | 2647 | C | PRO | A | 168 | 34.190 | −16.113 | −13.277 | 1.00 | 72.13 | C |
| ATOM | 2648 | O | PRO | A | 168 | 33.980 | −15.434 | −14.273 | 1.00 | 74.17 | O |
| ATOM | 2649 | N | SER | A | 169 | 33.333 | −16.243 | −12.263 | 1.00 | 70.78 | N |
| ATOM | 2651 | CA | SER | A | 169 | 32.029 | −15.649 | −12.196 | 1.00 | 70.36 | C |
| ATOM | 2653 | CB | SER | A | 169 | 31.448 | −16.185 | −10.906 | 1.00 | 69.09 | C |
| ATOM | 2656 | OG | SER | A | 169 | 30.162 | −15.740 | −10.627 | 1.00 | 69.59 | O |
| ATOM | 2658 | C | SER | A | 169 | 32.176 | −14.099 | −12.189 | 1.00 | 68.96 | C |
| ATOM | 2659 | O | SER | A | 169 | 31.369 | −13.354 | −12.767 | 1.00 | 69.80 | O |
| ATOM | 2660 | N | CYS | A | 170 | 33.235 | −13.645 | −11.522 | 1.00 | 66.34 | N |
| ATOM | 2662 | CA | CYS | A | 170 | 33.585 | −12.257 | −11.389 | 1.00 | 64.17 | C |
| ATOM | 2664 | CB | CYS | A | 170 | 35.009 | −12.194 | −10.803 | 1.00 | 62.64 | C |
| ATOM | 2667 | SG | CYS | A | 170 | 35.099 | −12.706 | −9.064 | 1.00 | 65.28 | S |
| ATOM | 2668 | C | CYS | A | 170 | 33.626 | −11.532 | −12.691 | 1.00 | 64.97 | C |
| ATOM | 2669 | O | CYS | A | 170 | 34.036 | −12.154 | −13.644 | 1.00 | 67.60 | O |
| ATOM | 2670 | N | PRO | A | 171 | 33.293 | −10.224 | −12.709 | 1.00 | 63.33 | N |
| ATOM | 2671 | CA | PRO | A | 171 | 33.587 | −9.309 | −13.840 | 1.00 | 63.95 | C |
| ATOM | 2673 | CB | PRO | A | 171 | 32.709 | −8.083 | −13.545 | 1.00 | 62.43 | C |
| ATOM | 2676 | CG | PRO | A | 171 | 32.618 | −8.081 | −12.137 | 1.00 | 60.40 | C |
| ATOM | 2679 | CD | PRO | A | 171 | 32.609 | −9.504 | −11.624 | 1.00 | 60.72 | C |
| ATOM | 2682 | C | PRO | A | 171 | 35.056 | −8.844 | −13.952 | 1.00 | 62.28 | C |
| ATOM | 2683 | O | PRO | A | 171 | 35.613 | −8.339 | −12.977 | 1.00 | 58.36 | O |
| ATOM | 2684 | N | ASN | A | 172 | 35.651 | −9.053 | −15.137 | 1.00 | 64.60 | N |
| ATOM | 2686 | CA | ASN | A | 172 | 37.014 | −8.626 | −15.469 | 1.00 | 64.03 | C |
| ATOM | 2688 | CB | ASN | A | 172 | 37.092 | −7.107 | −15.494 | 1.00 | 63.31 | C |
| ATOM | 2691 | CG | ASN | A | 172 | 36.067 | −6.493 | −16.417 | 1.00 | 64.86 | C |
| ATOM | 2692 | OD1 | ASN | A | 172 | 36.063 | −6.769 | −17.588 | 1.00 | 68.52 | O |
| ATOM | 2693 | ND2 | ASN | A | 172 | 35.183 | −5.689 | −15.884 | 1.00 | 63.98 | N |
| ATOM | 2696 | C | ASN | A | 172 | 38.072 | −9.188 | −14.525 | 1.00 | 62.11 | C |
| ATOM | 2697 | O | ASN | A | 172 | 39.141 | −8.611 | −14.391 | 1.00 | 60.44 | O |
| ATOM | 2698 | N | GLY | A | 173 | 37.760 | −10.330 | −13.891 | 1.00 | 61.90 | N |
| ATOM | 2700 | CA | GLY | A | 173 | 38.676 | −11.067 | −13.029 | 1.00 | 60.44 | C |
| ATOM | 2703 | C | GLY | A | 173 | 38.962 | −10.365 | −11.733 | 1.00 | 56.68 | C |
| ATOM | 2704 | O | GLY | A | 173 | 39.958 | −10.665 | −11.056 | 1.00 | 54.88 | O |
| ATOM | 2705 | N | SER | A | 174 | 38.081 | −9.436 | −11.392 | 1.00 | 54.92 | N |
| ATOM | 2707 | CA | SER | A | 174 | 38.350 | −8.556 | −10.289 | 1.00 | 53.27 | C |
| ATOM | 2709 | CB | SER | A | 174 | 38.094 | −7.084 | −10.657 | 1.00 | 54.14 | C |
| ATOM | 2712 | OG | SER | A | 174 | 37.408 | −6.922 | −11.890 | 1.00 | 61.12 | O |
| ATOM | 2714 | C | SER | A | 174 | 37.560 | −8.949 | −9.069 | 1.00 | 51.33 | C |
| ATOM | 2715 | O | SER | A | 174 | 36.354 | −9.118 | −9.128 | 1.00 | 51.96 | O |
| ATOM | 2716 | N | CYS | A | 175 | 38.277 | −9.140 | −7.965 | 1.00 | 50.14 | N |
| ATOM | 2718 | CA | CYS | A | 175 | 37.716 | −9.660 | −6.724 | 1.00 | 49.06 | C |
| ATOM | 2720 | CB | CYS | A | 175 | 37.668 | −11.161 | −6.785 | 1.00 | 51.08 | C |
| ATOM | 2723 | SG | CYS | A | 175 | 39.237 | −11.989 | −6.497 | 1.00 | 54.98 | S |
| ATOM | 2724 | C | CYS | A | 175 | 38.552 | −9.228 | −5.541 | 1.00 | 46.28 | C |
| ATOM | 2725 | O | CYS | A | 175 | 39.663 | −8.727 | −5.713 | 1.00 | 45.60 | O |
| ATOM | 2726 | N | TRP | A | 176 | 38.009 | −9.415 | −4.342 | 1.00 | 44.79 | N |
| ATOM | 2728 | CA | TRP | A | 176 | 38.693 | −9.052 | −3.078 | 1.00 | 42.74 | C |
| ATOM | 2730 | CB | TRP | A | 176 | 37.714 | −8.389 | −2.131 | 1.00 | 40.96 | C |
| ATOM | 2733 | CG | TRP | A | 176 | 37.119 | −7.038 | −2.642 | 1.00 | 40.42 | C |
| ATOM | 2734 | CD1 | TRP | A | 176 | 35.982 | −6.870 | −3.326 | 1.00 | 39.92 | C |
| ATOM | 2736 | NE1 | TRP | A | 176 | 35.755 | −5.544 | −3.560 | 1.00 | 41.12 | N |
| ATOM | 2738 | CE2 | TRP | A | 176 | 36.782 | −4.827 | −3.009 | 1.00 | 41.59 | C |
| ATOM | 2739 | CD2 | TRP | A | 176 | 37.639 | −5.735 | −2.393 | 1.00 | 38.66 | C |
| ATOM | 2740 | CE3 | TRP | A | 176 | 38.790 | −5.256 | −1.764 | 1.00 | 39.18 | C |
| ATOM | 2742 | CZ3 | TRP | A | 176 | 39.040 | −3.931 | −1.766 | 1.00 | 36.89 | C |
| ATOM | 2744 | CH2 | TRP | A | 176 | 38.161 | −3.054 | −2.364 | 1.00 | 40.57 | C |
| ATOM | 2746 | CZ2 | TRP | A | 176 | 37.023 | −3.483 | −3.010 | 1.00 | 40.72 | C |
| ATOM | 2748 | C | TRP | A | 176 | 39.264 | −10.290 | −2.369 | 1.00 | 43.56 | C |
| ATOM | 2749 | O | TRP | A | 176 | 39.952 | −10.188 | −1.370 | 1.00 | 42.95 | O |
| ATOM | 2750 | N | GLY | A | 177 | 38.981 | −11.465 | −2.907 | 1.00 | 45.37 | N |
| ATOM | 2752 | CA | GLY | A | 177 | 39.457 | −12.658 | −2.312 | 1.00 | 45.99 | C |
| ATOM | 2755 | C | GLY | A | 177 | 38.797 | −13.810 | −2.985 | 1.00 | 49.26 | C |
| ATOM | 2756 | O | GLY | A | 177 | 38.184 | −13.706 | −4.041 | 1.00 | 50.81 | O |
| ATOM | 2757 | N | ALA | A | 178 | 38.914 | −14.939 | −2.328 | 1.00 | 50.26 | N |
| ATOM | 2759 | CA | ALA | A | 178 | 38.527 | −16.179 | −2.908 | 1.00 | 53.28 | C |
| ATOM | 2761 | CB | ALA | A | 178 | 39.129 | −17.222 | −2.129 | 1.00 | 55.32 | C |
| ATOM | 2765 | C | ALA | A | 178 | 37.029 | −16.275 | −2.878 | 1.00 | 53.36 | C |
| ATOM | 2766 | O | ALA | A | 178 | 36.440 | −15.593 | −2.110 | 1.00 | 50.54 | O |
| ATOM | 2767 | N | GLY | A | 179 | 36.414 | −17.067 | −3.747 | 1.00 | 55.92 | N |
| ATOM | 2769 | CA | GLY | A | 179 | 34.974 | −17.248 | −3.736 | 1.00 | 57.34 | C |
| ATOM | 2772 | C | GLY | A | 179 | 34.100 | −16.268 | −4.495 | 1.00 | 56.65 | C |
| ATOM | 2773 | O | GLY | A | 179 | 34.480 | −15.136 | −4.759 | 1.00 | 53.03 | O |
| ATOM | 2774 | N | GLU | A | 180 | 32.925 | −16.772 | −4.873 | 1.00 | 59.51 | N |
| ATOM | 2776 | CA | GLU | A | 180 | 31.983 | −16.075 | −5.712 | 1.00 | 60.69 | C |
| ATOM | 2778 | CB | GLU | A | 180 | 30.822 | −16.977 | −6.100 | 1.00 | 64.23 | C |
| ATOM | 2781 | CG | GLU | A | 180 | 30.038 | −16.423 | −7.273 | 1.00 | 69.05 | C |
| ATOM | 2784 | CD | GLU | A | 180 | 28.984 | −17.373 | −7.882 | 1.00 | 79.34 | C |
| ATOM | 2785 | OE1 | GLU | A | 180 | 27.807 | −16.913 | −7.924 | 1.00 | 83.24 | O |
| ATOM | 2786 | OE2 | GLU | A | 180 | 29.287 | −18.553 | −8.346 | 1.00 | 85.62 | O |
| ATOM | 2787 | C | GLU | A | 180 | 31.425 | −14.874 | −4.965 | 1.00 | 58.20 | C |
| ATOM | 2788 | O | GLU | A | 180 | 31.056 | −13.872 | −5.585 | 1.00 | 55.67 | O |
| ATOM | 2789 | N | GLU | A | 181 | 31.338 | −15.008 | −3.639 | 1.00 | 57.72 | N |
| ATOM | 2791 | CA | GLU | A | 181 | 30.851 | −13.919 | −2.784 | 1.00 | 56.13 | C |
| ATOM | 2793 | CB | GLU | A | 181 | 30.543 | −14.432 | −1.393 | 1.00 | 56.53 | C |
| ATOM | 2796 | CG | GLU | A | 181 | 31.761 | −14.311 | −0.500 | 1.00 | 56.15 | C |
| ATOM | 2799 | CD | GLU | A | 181 | 31.764 | −15.221 | 0.695 | 1.00 | 58.93 | C |
| ATOM | 2800 | OE1 | GLU | A | 181 | 30.689 | −15.799 | 0.952 | 1.00 | 63.12 | O |
| ATOM | 2801 | OE2 | GLU | A | 181 | 32.849 | −15.341 | 1.348 | 1.00 | 58.06 | O |
| ATOM | 2802 | C | GLU | A | 181 | 31.802 | −12.698 | −2.640 | 1.00 | 52.53 | C |
| ATOM | 2803 | O | GLU | A | 181 | 31.385 | −11.655 | −2.106 | 1.00 | 51.74 | O |
| ATOM | 2804 | N | ASN | A | 182 | 33.061 | −12.845 | −3.073 | 1.00 | 51.13 | N |
| ATOM | 2806 | CA | ASN | A | 182 | 34.102 | −11.813 | −2.936 | 1.00 | 47.84 | C |
| ATOM | 2808 | CB | ASN | A | 182 | 35.396 | −12.431 | −2.392 | 1.00 | 46.56 | C |
| ATOM | 2811 | CG | ASN | A | 182 | 35.351 | −12.667 | −0.891 | 1.00 | 46.60 | C |
| ATOM | 2812 | OD1 | ASN | A | 182 | 34.761 | −11.922 | −0.093 | 1.00 | 45.34 | O |
| ATOM | 2813 | ND2 | ASN | A | 182 | 36.004 | −13.707 | −0.499 | 1.00 | 47.84 | N |
| ATOM | 2816 | C | ASN | A | 182 | 34.383 | −11.183 | −4.272 | 1.00 | 47.77 | C |
| ATOM | 2817 | O | ASN | A | 182 | 35.383 | −10.470 | −4.441 | 1.00 | 45.91 | O |
| ATOM | 2818 | N | CYS | A | 183 | 33.503 | −11.480 | −5.236 | 1.00 | 50.13 | N |
| ATOM | 2820 | CA | CYS | A | 183 | 33.545 | −10.849 | −6.567 | 1.00 | 51.09 | C |
| ATOM | 2822 | CB | CYS | A | 183 | 32.578 | −11.504 | −7.579 | 1.00 | 53.48 | C |
| ATOM | 2825 | SG | CYS | A | 183 | 33.164 | −13.146 | −8.334 | 1.00 | 63.64 | S |
| ATOM | 2826 | C | CYS | A | 183 | 33.204 | −9.376 | −6.289 | 1.00 | 48.00 | C |
| ATOM | 2827 | O | CYS | A | 183 | 32.345 | −9.091 | −5.454 | 1.00 | 45.84 | O |
| ATOM | 2828 | N | GLN | A | 184 | 33.937 | −8.507 | −6.987 | 1.00 | 46.92 | N |
| ATOM | 2830 | CA | GLN | A | 184 | 33.621 | −7.097 | −7.082 | 1.00 | 47.29 | C |
| ATOM | 2832 | CB | GLN | A | 184 | 34.702 | −6.286 | −7.837 | 1.00 | 45.60 | C |
| ATOM | 2835 | CG | GLN | A | 184 | 34.313 | −4.821 | −8.086 | 1.00 | 46.53 | C |
| ATOM | 2838 | CD | GLN | A | 184 | 35.490 | −3.732 | −8.235 | 1.00 | 45.39 | C |
| ATOM | 2839 | OE1 | GLN | A | 184 | 36.557 | −3.993 | −8.815 | 1.00 | 43.19 | O |
| ATOM | 2840 | NE2 | GLN | A | 184 | 35.226 | −2.509 | −7.721 | 1.00 | 41.52 | N |
| ATOM | 2843 | C | GLN | A | 184 | 32.209 | −6.922 | −7.643 | 1.00 | 49.85 | C |
| ATOM | 2844 | O | GLN | A | 184 | 31.887 | −7.418 | −8.732 | 1.00 | 53.03 | O |
| ATOM | 2845 | N | LYS | A | 185 | 31.342 | −6.271 | −6.868 | 1.00 | 49.27 | N |
| ATOM | 2847 | CA | LYS | A | 185 | 30.086 | −5.798 | −7.441 | 1.00 | 51.63 | C |
| ATOM | 2849 | CB | LYS | A | 185 | 29.023 | −5.620 | −6.343 | 1.00 | 52.06 | C |
| ATOM | 2852 | CG | LYS | A | 185 | 28.517 | −6.901 | −5.629 | 1.00 | 53.93 | C |
| ATOM | 2855 | CD | LYS | A | 185 | 28.156 | −6.583 | −4.065 | 1.00 | 53.03 | C |
| ATOM | 2858 | CE | LYS | A | 185 | 28.695 | −7.612 | −3.059 | 1.00 | 49.07 | C |
| ATOM | 2861 | NZ | LYS | A | 185 | 27.572 | −8.323 | −2.540 | 1.00 | 54.03 | N |
| ATOM | 2865 | C | LYS | A | 185 | 30.318 | −4.457 | −8.218 | 1.00 | 50.18 | C |
| ATOM | 2866 | O | LYS | A | 185 | 30.983 | −3.581 | −7.726 | 1.00 | 49.13 | O |
| ATOM | 2867 | N | LEU | A | 186 | 29.780 | −4.279 | −9.412 | 1.00 | 52.33 | N |
| ATOM | 2869 | CA | LEU | A | 186 | 29.967 | −3.001 | −10.114 | 1.00 | 52.28 | C |
| ATOM | 2871 | CB | LEU | A | 186 | 30.613 | −3.131 | −11.493 | 1.00 | 53.18 | C |
| ATOM | 2874 | CG | LEU | A | 186 | 32.019 | −3.758 | −11.492 | 1.00 | 52.36 | C |
| ATOM | 2876 | CD1 | LEU | A | 186 | 32.537 | −4.107 | −12.914 | 1.00 | 54.00 | C |
| ATOM | 2880 | CD2 | LEU | A | 186 | 33.021 | −2.930 | −10.768 | 1.00 | 48.37 | C |
| ATOM | 2884 | C | LEU | A | 186 | 28.654 | −2.280 | −10.199 | 1.00 | 54.66 | C |
| ATOM | 2885 | O | LEU | A | 186 | 27.611 | −2.903 | −10.407 | 1.00 | 56.57 | O |
| ATOM | 2886 | N | THR | A | 187 | 28.725 | −0.964 | −9.972 | 1.00 | 54.57 | N |
| ATOM | 2888 | CA | THR | A | 187 | 27.587 | −0.095 | −10.035 | 1.00 | 57.60 | C |
| ATOM | 2890 | CB | THR | A | 187 | 27.054 | 0.099 | −8.609 | 1.00 | 56.84 | C |
| ATOM | 2892 | OG1 | THR | A | 187 | 28.131 | 0.506 | −7.763 | 1.00 | 56.81 | O |
| ATOM | 2894 | CG2 | THR | A | 187 | 26.549 | −1.189 | −7.960 | 1.00 | 55.84 | C |
| ATOM | 2898 | C | THR | A | 187 | 27.833 | 1.310 | −10.662 | 1.00 | 59.12 | C |
| ATOM | 2899 | O | THR | A | 187 | 26.885 | 2.106 | −10.684 | 1.00 | 60.17 | O |
| ATOM | 2900 | N | LYS | A | 188 | 29.051 | 1.629 | −11.153 | 1.00 | 59.07 | N |
| ATOM | 2902 | CA | LYS | A | 188 | 29.350 | 2.969 | −11.776 | 1.00 | 60.11 | C |
| ATOM | 2904 | CB | LYS | A | 188 | 30.430 | 3.762 | −10.957 | 1.00 | 57.60 | C |
| ATOM | 2907 | CG | LYS | A | 188 | 30.704 | 5.336 | −11.208 | 1.00 | 53.08 | C |
| ATOM | 2910 | CD | LYS | A | 188 | 31.704 | 6.033 | −10.139 | 1.00 | 50.46 | C |
| ATOM | 2913 | CE | LYS | A | 188 | 32.508 | 7.420 | −10.695 | 1.00 | 62.36 | C |
| ATOM | 2916 | NZ | LYS | A | 188 | 32.092 | 9.158 | −10.925 | 1.00 | 56.57 | N |
| ATOM | 2920 | C | LYS | A | 188 | 29.771 | 2.735 | −13.239 | 1.00 | 63.34 | C |
| ATOM | 2921 | O | LYS | A | 188 | 29.111 | 3.149 | −14.157 | 1.00 | 66.50 | O |
| ATOM | 2922 | N | ILE | A | 189 | 30.843 | 1.998 | −13.440 | 1.00 | 63.68 | N |
| ATOM | 2924 | CA | ILE | A | 189 | 31.424 | 1.690 | −14.765 | 1.00 | 67.03 | C |
| ATOM | 2926 | CB | ILE | A | 189 | 32.738 | 0.990 | −14.377 | 1.00 | 65.88 | C |
| ATOM | 2928 | CG1 | ILE | A | 189 | 33.814 | 2.078 | −14.376 | 1.00 | 68.15 | C |
| ATOM | 2931 | CD1 | ILE | A | 189 | 34.948 | 1.628 | −13.491 | 1.00 | 70.37 | C |
| ATOM | 2935 | CG2 | ILE | A | 189 | 33.099 | −0.237 | −15.182 | 1.00 | 66.88 | C |
| ATOM | 2939 | C | ILE | A | 189 | 30.607 | 0.832 | −15.788 | 1.00 | 70.02 | C |
| ATOM | 2940 | O | ILE | A | 189 | 30.992 | 0.629 | −16.920 | 1.00 | 72.11 | O |
| ATOM | 2941 | N | ILE | A | 190 | 29.476 | 0.336 | −15.338 | 1.00 | 70.44 | N |
| ATOM | 2943 | CA | ILE | A | 190 | 28.701 | −0.687 | −15.994 | 1.00 | 72.98 | C |
| ATOM | 2945 | CB | ILE | A | 190 | 28.515 | −1.794 | −14.937 | 1.00 | 70.63 | C |
| ATOM | 2947 | CG1 | ILE | A | 190 | 29.637 | −2.783 | −15.140 | 1.00 | 71.22 | C |
| ATOM | 2950 | CD1 | ILE | A | 190 | 29.307 | −4.255 | −14.645 | 1.00 | 76.22 | C |
| ATOM | 2954 | CG2 | ILE | A | 190 | 27.125 | −2.377 | −14.917 | 1.00 | 71.99 | C |
| ATOM | 2958 | C | ILE | A | 190 | 27.366 | −0.090 | −16.413 | 1.00 | 76.24 | C |
| ATOM | 2959 | O | ILE | A | 190 | 26.564 | −0.726 | −17.119 | 1.00 | 79.79 | O |
| ATOM | 2960 | N | CYS | A | 191 | 27.136 | 1.143 | −15.996 | 1.00 | 75.30 | N |
| ATOM | 2962 | CA | CYS | A | 191 | 25.844 | 1.785 | −16.157 | 1.00 | 78.23 | C |
| ATOM | 2964 | CB | CYS | A | 191 | 25.752 | 2.923 | −15.123 | 1.00 | 76.32 | C |
| ATOM | 2967 | SG | CYS | A | 191 | 26.102 | 2.238 | −13.446 | 1.00 | 74.45 | S |
| ATOM | 2968 | C | CYS | A | 191 | 25.605 | 2.255 | −17.601 | 1.00 | 81.91 | C |
| ATOM | 2969 | O | CYS | A | 191 | 26.543 | 2.581 | −18.338 | 1.00 | 82.17 | O |
| ATOM | 2970 | N | ALA | A | 192 | 24.346 | 2.227 | −18.012 | 1.00 | 84.89 | N |
| ATOM | 2972 | CA | ALA | A | 192 | 23.966 | 2.868 | −19.251 | 1.00 | 89.58 | C |
| ATOM | 2974 | CB | ALA | A | 192 | 22.450 | 2.842 | −19.400 | 1.00 | 93.30 | C |
| ATOM | 2978 | C | ALA | A | 192 | 24.506 | 4.339 | −19.275 | 1.00 | 89.64 | C |
| ATOM | 2979 | O | ALA | A | 192 | 24.639 | 4.980 | −18.201 | 1.00 | 85.53 | O |
| ATOM | 2980 | N | GLN | A | 193 | 24.836 | 4.846 | −20.484 | 1.00 | 93.10 | N |
| ATOM | 2982 | CA | GLN | A | 193 | 25.353 | 6.215 | −20.616 | 1.00 | 94.09 | C |
| ATOM | 2984 | CB | GLN | A | 193 | 25.538 | 6.634 | −22.098 | 1.00 | 99.22 | C |
| ATOM | 2987 | CG | GLN | A | 193 | 26.163 | 8.062 | −22.297 | 1.00 | 99.92 | C |
| ATOM | 2990 | CD | GLN | A | 193 | 25.933 | 8.638 | −23.692 | 1.00 | 108.02 | C |
| ATOM | 2991 | OE1 | GLN | A | 193 | 25.886 | 7.901 | −24.675 | 1.00 | 115.13 | O |
| ATOM | 2992 | NE2 | GLN | A | 193 | 25.800 | 9.955 | −23.788 | 1.00 | 112.64 | N |
| ATOM | 2995 | C | GLN | A | 193 | 24.388 | 7.177 | −19.901 | 1.00 | 94.51 | C |
| ATOM | 2996 | O | GLN | A | 193 | 24.827 | 8.100 | −19.208 | 1.00 | 92.51 | O |
| ATOM | 2997 | N | GLN | A | 194 | 23.086 | 6.891 | −20.050 | 1.00 | 97.35 | N |
| ATOM | 2999 | CA | GLN | A | 194 | 21.973 | 7.735 | −19.591 | 1.00 | 99.21 | C |
| ATOM | 3001 | CB | GLN | A | 194 | 20.667 | 7.116 | −20.142 | 1.00 | 103.46 | C |
| ATOM | 3008 | C | GLN | A | 194 | 21.839 | 8.015 | −18.057 | 1.00 | 95.71 | C |
| ATOM | 3009 | O | GLN | A | 194 | 21.013 | 8.809 | −17.645 | 1.00 | 97.16 | O |
| ATOM | 3010 | N | CYS | A | 195 | 22.633 | 7.364 | −17.216 | 1.00 | 91.62 | N |
| ATOM | 3012 | CA | CYS | A | 195 | 22.490 | 7.526 | −15.791 | 1.00 | 88.83 | C |
| ATOM | 3014 | CB | CYS | A | 195 | 22.917 | 6.292 | −15.017 | 1.00 | 85.27 | C |
| ATOM | 3017 | SG | CYS | A | 195 | 21.902 | 4.815 | −15.196 | 1.00 | 90.56 | S |
| ATOM | 3018 | C | CYS | A | 195 | 23.412 | 8.598 | −15.346 | 1.00 | 86.94 | C |
| ATOM | 3019 | O | CYS | A | 195 | 24.607 | 8.599 | −15.680 | 1.00 | 85.07 | O |
| ATOM | 3020 | N | SER | A | 196 | 22.858 | 9.456 | −14.507 | 1.00 | 87.22 | N |
| ATOM | 3022 | CA | SER | A | 196 | 23.627 | 10.438 | −13.814 | 1.00 | 85.77 | C |
| ATOM | 3024 | CB | SER | A | 196 | 22.648 | 11.354 | −13.047 | 1.00 | 87.93 | C |
| ATOM | 3027 | OG | SER | A | 196 | 22.540 | 11.018 | −11.651 | 1.00 | 86.76 | O |
| ATOM | 3029 | C | SER | A | 196 | 24.677 | 9.843 | −12.839 | 1.00 | 81.16 | C |
| ATOM | 3030 | O | SER | A | 196 | 25.573 | 10.557 | −12.395 | 1.00 | 81.13 | O |
| ATOM | 3031 | N | GLY | A | 197 | 24.538 | 8.580 | −12.438 | 1.00 | 78.73 | N |
| ATOM | 3033 | CA | GLY | A | 197 | 25.334 | 8.000 | −11.342 | 1.00 | 73.56 | C |
| ATOM | 3036 | C | GLY | A | 197 | 24.830 | 6.601 | −11.007 | 1.00 | 72.09 | C |
| ATOM | 3037 | O | GLY | A | 197 | 23.864 | 6.111 | −11.623 | 1.00 | 75.94 | O |
| ATOM | 3038 | N | ARG | A | 198 | 25.480 | 5.936 | −10.068 | 1.00 | 67.34 | N |
| ATOM | 3040 | CA | ARG | A | 198 | 25.152 | 4.530 | −9.712 | 1.00 | 65.41 | C |
| ATOM | 3042 | CB | ARG | A | 198 | 24.991 | 4.402 | −8.192 | 1.00 | 63.68 | C |
| ATOM | 3045 | CG | ARG | A | 198 | 26.212 | 4.737 | −7.348 | 1.00 | 57.45 | C |
| ATOM | 3048 | CD | ARG | A | 198 | 27.428 | 4.201 | −7.931 | 1.00 | 54.35 | C |
| ATOM | 3051 | NE | ARG | A | 198 | 28.605 | 4.632 | −7.227 | 1.00 | 49.75 | N |
| ATOM | 3053 | CZ | ARG | A | 198 | 29.670 | 3.892 | −7.060 | 1.00 | 48.00 | C |
| ATOM | 3054 | NH1 | ARG | A | 198 | 29.696 | 2.647 | −7.498 | 1.00 | 51.64 | N |
| ATOM | 3057 | NH2 | ARG | A | 198 | 30.717 | 4.384 | −6.450 | 1.00 | 45.20 | N |
| ATOM | 3060 | C | ARG | A | 198 | 23.916 | 3.831 | −10.309 | 1.00 | 67.96 | C |
| ATOM | 3061 | O | ARG | A | 198 | 22.865 | 4.429 | −10.482 | 1.00 | 69.74 | O |
| ATOM | 3062 | N | CYS | A | 199 | 24.066 | 2.517 | −10.533 | 1.00 | 68.17 | N |
| ATOM | 3064 | CA | CYS | A | 199 | 22.980 | 1.584 | −11.035 | 1.00 | 70.76 | C |
| ATOM | 3066 | CB | CYS | A | 199 | 23.028 | 1.453 | −12.552 | 1.00 | 73.17 | C |
| ATOM | 3069 | SG | CYS | A | 199 | 24.620 | 0.814 | −13.123 | 1.00 | 70.86 | S |
| ATOM | 3070 | C | CYS | A | 199 | 23.083 | 0.184 | −10.385 | 1.00 | 68.65 | C |
| ATOM | 3071 | O | CYS | A | 199 | 23.915 | −0.044 | −9.450 | 1.00 | 65.70 | O |
| ATOM | 3072 | N | ARG | A | 200 | 22.214 | −0.730 | −10.837 | 1.00 | 70.83 | N |
| ATOM | 3074 | CA | ARG | A | 200 | 22.179 | −2.148 | −10.359 | 1.00 | 69.39 | C |
| ATOM | 3076 | CB | ARG | A | 200 | 21.098 | −2.232 | −9.299 | 1.00 | 70.45 | C |
| ATOM | 3079 | CG | ARG | A | 200 | 19.798 | −1.633 | −9.740 | 1.00 | 73.74 | C |
| ATOM | 3082 | CD | ARG | A | 200 | 18.746 | −1.838 | −8.754 | 1.00 | 76.36 | C |
| ATOM | 3085 | NE | ARG | A | 200 | 17.429 | −1.744 | −9.348 | 1.00 | 82.84 | N |
| ATOM | 3087 | CZ | ARG | A | 200 | 16.411 | −2.522 | −9.015 | 1.00 | 86.81 | C |
| ATOM | 3088 | NH1 | ARG | A | 200 | 16.540 | −3.467 | −8.083 | 1.00 | 85.55 | N |
| ATOM | 3091 | NH2 | ARG | A | 200 | 15.245 | −2.346 | −9.616 | 1.00 | 92.94 | N |
| ATOM | 3094 | C | ARG | A | 200 | 21.932 | −3.214 | −11.467 | 1.00 | 71.45 | C |
| ATOM | 3095 | O | ARG | A | 200 | 21.777 | −4.416 | −11.236 | 1.00 | 71.97 | O |
| ATOM | 3096 | N | GLY | A | 201 | 21.869 | −2.714 | −12.673 | 1.00 | 73.31 | N |
| ATOM | 3098 | CA | GLY | A | 201 | 21.586 | −3.476 | −13.847 | 1.00 | 77.37 | C |
| ATOM | 3101 | C | GLY | A | 201 | 22.227 | −2.705 | −14.954 | 1.00 | 78.01 | C |
| ATOM | 3102 | O | GLY | A | 201 | 22.616 | −1.565 | −14.732 | 1.00 | 76.98 | O |
| ATOM | 3103 | N | LYS | A | 202 | 22.386 | −3.322 | −16.117 | 1.00 | 80.54 | N |
| ATOM | 3105 | CA | LYS | A | 202 | 23.135 | −2.699 | −17.185 | 1.00 | 81.72 | C |
| ATOM | 3107 | CB | LYS | A | 202 | 23.959 | −3.720 | −18.012 | 1.00 | 81.81 | C |
| ATOM | 3114 | C | LYS | A | 202 | 22.129 | −1.884 | −17.999 | 1.00 | 86.87 | C |
| ATOM | 3115 | O | LYS | A | 202 | 22.526 | −1.153 | −18.906 | 1.00 | 88.82 | O |
| ATOM | 3116 | N | SER | A | 203 | 20.842 | −1.953 | −17.613 | 1.00 | 89.89 | N |
| ATOM | 3118 | CA | SER | A | 203 | 19.704 | −1.280 | −18.316 | 1.00 | 94.97 | C |
| ATOM | 3120 | CB | SER | A | 203 | 18.414 | −2.065 | −18.031 | 1.00 | 98.06 | C |
| ATOM | 3123 | OG | SER | A | 203 | 18.209 | −2.178 | −16.619 | 1.00 | 96.46 | O |
| ATOM | 3125 | C | SER | A | 203 | 19.442 | 0.215 | −17.938 | 1.00 | 94.60 | C |
| ATOM | 3126 | O | SER | A | 203 | 19.990 | 0.716 | −16.921 | 1.00 | 89.96 | O |
| ATOM | 3127 | N | PRO | A | 204 | 18.628 | 0.908 | −18.767 | 1.00 | 99.08 | N |
| ATOM | 3128 | CA | PRO | A | 204 | 18.061 | 2.235 | −18.420 | 1.00 | 99.90 | C |
| ATOM | 3130 | CB | PRO | A | 204 | 17.375 | 2.695 | −19.710 | 1.00 | 105.95 | C |
| ATOM | 3133 | CG | PRO | A | 204 | 17.293 | 1.497 | −20.624 | 1.00 | 108.00 | C |
| ATOM | 3136 | CD | PRO | A | 204 | 18.277 | 0.493 | −20.142 | 1.00 | 103.68 | C |
| ATOM | 3139 | C | PRO | A | 204 | 17.048 | 2.246 | −17.273 | 1.00 | 100.16 | C |
| ATOM | 3140 | O | PRO | A | 204 | 17.060 | 3.133 | −16.434 | 1.00 | 98.31 | O |
| ATOM | 3141 | N | SER | A | 205 | 16.173 | 1.267 | −17.206 | 1.00 | 102.97 | N |
| ATOM | 3143 | CA | SER | A | 205 | 15.376 | 1.118 | −15.992 | 1.00 | 103.18 | C |
| ATOM | 3145 | CB | SER | A | 205 | 14.395 | −0.004 | −16.148 | 1.00 | 107.04 | C |
| ATOM | 3148 | OG | SER | A | 205 | 15.123 | −1.122 | −16.565 | 1.00 | 106.57 | O |
| ATOM | 3150 | C | SER | A | 205 | 16.237 | 0.823 | −14.770 | 1.00 | 97.64 | C |
| ATOM | 3151 | O | SER | A | 205 | 15.875 | 1.164 | −13.651 | 1.00 | 97.84 | O |
| ATOM | 3152 | N | ASP | A | 206 | 17.401 | 0.227 | −14.948 | 1.00 | 94.14 | N |
| ATOM | 3154 | CA | ASP | A | 206 | 18.244 | −0.075 | −13.781 | 1.00 | 88.73 | C |
| ATOM | 3156 | CB | ASP | A | 206 | 19.266 | −1.183 | −14.155 | 1.00 | 86.17 | C |
| ATOM | 3162 | C | ASP | A | 206 | 18.926 | 1.144 | −13.099 | 1.00 | 84.68 | C |
| ATOM | 3163 | O | ASP | A | 206 | 19.810 | 0.969 | −12.300 | 1.00 | 80.94 | O |
| ATOM | 3164 | N | CYS | A | 207 | 18.545 | 2.373 | −13.378 | 1.00 | 86.84 | N |
| ATOM | 3166 | CA | CYS | A | 207 | 19.279 | 3.512 | −12.786 | 1.00 | 84.84 | C |
| ATOM | 3168 | CB | CYS | A | 207 | 19.006 | 4.782 | −13.567 | 1.00 | 88.15 | C |
| ATOM | 3171 | SG | CYS | A | 207 | 19.830 | 4.949 | −15.177 | 1.00 | 93.16 | S |
| ATOM | 3172 | C | CYS | A | 207 | 18.945 | 3.809 | −11.311 | 1.00 | 82.80 | C |
| ATOM | 3173 | O | CYS | A | 207 | 17.821 | 3.524 | −10.885 | 1.00 | 85.48 | O |
| ATOM | 3174 | N | CYS | A | 208 | 19.915 | 4.385 | −10.561 | 1.00 | 78.90 | N |
| ATOM | 3176 | CA | CYS | A | 208 | 19.749 | 4.786 | −9.117 | 1.00 | 76.99 | C |
| ATOM | 3178 | CB | CYS | A | 208 | 20.959 | 4.473 | −8.200 | 1.00 | 71.82 | C |
| ATOM | 3181 | SG | CYS | A | 208 | 21.335 | 2.712 | −7.852 | 1.00 | 71.46 | S |
| ATOM | 3182 | C | CYS | A | 208 | 19.496 | 6.275 | −9.006 | 1.00 | 77.75 | C |
| ATOM | 3183 | O | CYS | A | 208 | 20.107 | 7.074 | −9.710 | 1.00 | 76.69 | O |
| ATOM | 3184 | N | HIS | A | 209 | 18.607 | 6.637 | −8.092 | 1.00 | 79.28 | N |
| ATOM | 3186 | CA | HIS | A | 209 | 18.280 | 8.040 | −7.879 | 1.00 | 81.28 | C |
| ATOM | 3188 | CB | HIS | A | 209 | 17.372 | 8.223 | −6.680 | 1.00 | 82.57 | C |
| ATOM | 3191 | CG | HIS | A | 209 | 16.907 | 9.628 | −6.497 | 1.00 | 86.38 | C |
| ATOM | 3192 | ND1 | HIS | A | 209 | 15.853 | 10.170 | −7.198 | 1.00 | 94.24 | N |
| ATOM | 3194 | CE1 | HIS | A | 209 | 15.655 | 11.421 | −6.809 | 1.00 | 96.56 | C |
| ATOM | 3196 | NE2 | HIS | A | 209 | 16.568 | 11.718 | −5.902 | 1.00 | 91.93 | N |
| ATOM | 3198 | CD2 | HIS | A | 209 | 17.357 | 10.610 | −5.691 | 1.00 | 86.90 | C |
| ATOM | 3200 | C | HIS | A | 209 | 19.551 | 8.845 | −7.694 | 1.00 | 78.10 | C |
| ATOM | 3201 | O | HIS | A | 209 | 20.510 | 8.343 | −7.144 | 1.00 | 73.89 | O |
| ATOM | 3202 | N | ASN | A | 210 | 19.543 | 10.089 | −8.168 | 1.00 | 80.56 | N |
| ATOM | 3204 | CA | ASN | A | 210 | 20.717 | 10.936 | −8.164 | 1.00 | 78.95 | C |
| ATOM | 3206 | CB | ASN | A | 210 | 20.251 | 12.335 | −8.576 | 1.00 | 83.73 | C |
| ATOM | 3209 | CG | ASN | A | 210 | 21.408 | 13.355 | −8.850 | 1.00 | 85.95 | C |
| ATOM | 3210 | OD1 | ASN | A | 210 | 22.542 | 13.249 | −8.323 | 1.00 | 81.95 | O |
| ATOM | 3211 | ND2 | ASN | A | 210 | 21.065 | 14.416 | −9.643 | 1.00 | 92.19 | N |
| ATOM | 3214 | C | ASN | A | 210 | 21.360 | 10.886 | −6.758 | 1.00 | 75.18 | C |
| ATOM | 3215 | O | ASN | A | 210 | 22.583 | 10.777 | −6.639 | 1.00 | 72.10 | O |
| ATOM | 3216 | N | GLN | A | 211 | 20.516 | 10.873 | −5.716 | 1.00 | 75.07 | N |
| ATOM | 3218 | CA | GLN | A | 211 | 20.930 | 10.869 | −4.335 | 1.00 | 71.75 | C |
| ATOM | 3220 | CB | GLN | A | 211 | 19.728 | 11.029 | −3.403 | 1.00 | 73.99 | C |
| ATOM | 3223 | CG | GLN | A | 211 | 18.918 | 12.325 | −3.575 | 1.00 | 77.34 | C |
| ATOM | 3226 | CD | GLN | A | 211 | 19.620 | 13.572 | −3.094 | 1.00 | 73.73 | C |
| ATOM | 3227 | OE1 | GLN | A | 211 | 20.318 | 13.552 | −2.113 | 1.00 | 70.44 | O |
| ATOM | 3228 | NE2 | GLN | A | 211 | 19.431 | 14.657 | −3.802 | 1.00 | 77.44 | N |
| ATOM | 3231 | C | GLN | A | 211 | 21.690 | 9.671 | −3.809 | 1.00 | 68.89 | C |
| ATOM | 3232 | O | GLN | A | 211 | 22.103 | 9.730 | −2.654 | 1.00 | 68.37 | O |
| ATOM | 3233 | N | CYS | A | 212 | 21.895 | 8.585 | −4.561 | 1.00 | 68.32 | N |
| ATOM | 3235 | CA | CYS | A | 212 | 22.445 | 7.341 | −3.952 | 1.00 | 65.45 | C |
| ATOM | 3237 | CB | CYS | A | 212 | 21.877 | 6.071 | −4.588 | 1.00 | 66.69 | C |
| ATOM | 3240 | SG | CYS | A | 212 | 20.095 | 5.933 | −4.285 | 1.00 | 74.19 | S |
| ATOM | 3241 | C | CYS | A | 212 | 23.942 | 7.262 | −3.984 | 1.00 | 61.86 | C |
| ATOM | 3242 | O | CYS | A | 212 | 24.613 | 7.907 | −4.833 | 1.00 | 61.22 | O |
| ATOM | 3243 | N | ALA | A | 213 | 24.468 | 6.458 | −3.056 | 1.00 | 59.19 | N |
| ATOM | 3245 | CA | ALA | A | 213 | 25.877 | 6.519 | −2.765 | 1.00 | 56.34 | C |
| ATOM | 3247 | CB | ALA | A | 213 | 26.105 | 6.720 | −1.392 | 1.00 | 55.01 | C |
| ATOM | 3251 | C | ALA | A | 213 | 26.611 | 5.295 | −3.245 | 1.00 | 54.74 | C |
| ATOM | 3252 | O | ALA | A | 213 | 27.718 | 5.456 | −3.821 | 1.00 | 54.91 | O |
| ATOM | 3253 | N | ALA | A | 214 | 26.052 | 4.111 | −3.043 | 1.00 | 54.09 | N |
| ATOM | 3255 | CA | ALA | A | 214 | 26.786 | 2.893 | −3.436 | 1.00 | 52.51 | C |
| ATOM | 3257 | CB | ALA | A | 214 | 26.982 | 2.055 | −2.300 | 1.00 | 50.79 | C |
| ATOM | 3261 | C | ALA | A | 214 | 26.086 | 2.088 | −4.486 | 1.00 | 55.33 | C |
| ATOM | 3262 | O | ALA | A | 214 | 26.726 | 1.278 | −5.148 | 1.00 | 55.72 | O |
| ATOM | 3263 | N | GLY | A | 215 | 24.783 | 2.329 | −4.653 | 1.00 | 58.27 | N |
| ATOM | 3265 | CA | GLY | A | 215 | 23.914 | 1.484 | −5.445 | 1.00 | 60.72 | C |
| ATOM | 3268 | C | GLY | A | 215 | 22.512 | 1.447 | −4.858 | 1.00 | 63.18 | C |
| ATOM | 3269 | O | GLY | A | 215 | 22.195 | 2.220 | −3.951 | 1.00 | 62.63 | O |
| ATOM | 3270 | N | CYS | A | 216 | 21.657 | 0.574 | −5.394 | 1.00 | 65.69 | N |
| ATOM | 3272 | CA | CYS | A | 216 | 20.258 | 0.586 | −4.964 | 1.00 | 69.24 | C |
| ATOM | 3274 | CB | CYS | A | 216 | 19.535 | 1.716 | −5.650 | 1.00 | 72.29 | C |
| ATOM | 3277 | SG | CYS | A | 216 | 19.657 | 1.623 | −7.430 | 1.00 | 74.13 | S |
| ATOM | 3278 | C | CYS | A | 216 | 19.447 | −0.665 | −5.227 | 1.00 | 71.77 | C |
| ATOM | 3279 | O | CYS | A | 216 | 19.886 | −1.583 | −5.916 | 1.00 | 71.93 | O |
| ATOM | 3280 | N | THR | A | 217 | 18.233 | −0.626 | −4.696 | 1.00 | 73.98 | N |
| ATOM | 3282 | CA | THR | A | 217 | 17.304 | −1.724 | −4.665 | 1.00 | 76.58 | C |
| ATOM | 3284 | CB | THR | A | 217 | 16.933 | −1.861 | −3.231 | 1.00 | 76.60 | C |
| ATOM | 3286 | OG1 | THR | A | 217 | 18.073 | −2.376 | −2.558 | 1.00 | 71.61 | O |
| ATOM | 3288 | CG2 | THR | A | 217 | 15.797 | −2.863 | −2.987 | 1.00 | 81.96 | C |
| ATOM | 3292 | C | THR | A | 217 | 16.089 | −1.360 | −5.465 | 1.00 | 81.10 | C |
| ATOM | 3293 | O | THR | A | 217 | 15.039 | −1.958 | −5.310 | 1.00 | 84.07 | O |
| ATOM | 3294 | N | GLY | A | 218 | 16.232 | −0.341 | −6.305 | 1.00 | 81.30 | N |
| ATOM | 3296 | CA | GLY | A | 218 | 15.083 | 0.262 | −6.937 | 1.00 | 86.06 | C |
| ATOM | 3299 | C | GLY | A | 218 | 15.298 | 1.697 | −7.356 | 1.00 | 85.69 | C |
| ATOM | 3300 | O | GLY | A | 218 | 16.323 | 2.320 | −7.062 | 1.00 | 81.26 | O |
| ATOM | 3301 | N | PRO | A | 219 | 14.270 | 2.229 | −8.003 | 1.00 | 90.50 | N |
| ATOM | 3302 | CA | PRO | A | 219 | 14.353 | 3.496 | −8.749 | 1.00 | 91.95 | C |
| ATOM | 3304 | CB | PRO | A | 219 | 12.956 | 3.600 | −9.384 | 1.00 | 98.27 | C |
| ATOM | 3307 | CG | PRO | A | 219 | 12.042 | 2.762 | −8.489 | 1.00 | 99.76 | C |
| ATOM | 3310 | CD | PRO | A | 219 | 12.909 | 1.656 | −8.018 | 1.00 | 95.47 | C |
| ATOM | 3313 | C | PRO | A | 219 | 14.624 | 4.769 | −7.946 | 1.00 | 90.33 | C |
| ATOM | 3314 | O | PRO | A | 219 | 15.405 | 5.591 | −8.349 | 1.00 | 88.23 | O |
| ATOM | 3315 | N | ARG | A | 220 | 13.981 | 4.904 | −6.810 | 1.00 | 91.91 | N |
| ATOM | 3317 | CA | ARG | A | 220 | 13.738 | 6.217 | −6.233 | 1.00 | 93.99 | C |
| ATOM | 3319 | CB | ARG | A | 220 | 12.218 | 6.360 | −5.904 | 1.00 | 100.22 | C |
| ATOM | 3322 | CG | ARG | A | 220 | 11.634 | 5.167 | −5.100 | 1.00 | 102.19 | C |
| ATOM | 3325 | CD | ARG | A | 220 | 10.103 | 5.144 | −4.836 | 1.00 | 109.43 | C |
| ATOM | 3328 | NE | ARG | A | 220 | 9.740 | 3.785 | −4.386 | 1.00 | 111.00 | N |
| ATOM | 3330 | CZ | ARG | A | 220 | 8.505 | 3.358 | −4.091 | 1.00 | 115.28 | C |
| ATOM | 3331 | NH1 | ARG | A | 220 | 7.446 | 4.179 | −4.177 | 1.00 | 118.66 | N |
| ATOM | 3334 | NH2 | ARG | A | 220 | 8.347 | 2.087 | −3.696 | 1.00 | 114.53 | N |
| ATOM | 3337 | C | ARG | A | 220 | 14.631 | 6.612 | −5.014 | 1.00 | 89.71 | C |
| ATOM | 3338 | O | ARG | A | 220 | 15.450 | 5.829 | −4.543 | 1.00 | 86.23 | O |
| ATOM | 3339 | N | GLU | A | 221 | 14.468 | 7.873 | −4.587 | 1.00 | 90.79 | N |
| ATOM | 3341 | CA | GLU | A | 221 | 14.998 | 8.469 | −3.350 | 1.00 | 88.14 | C |
| ATOM | 3343 | CB | GLU | A | 221 | 14.054 | 9.612 | −2.885 | 1.00 | 92.21 | C |
| ATOM | 3346 | CG | GLU | A | 221 | 13.975 | 10.853 | −3.765 | 1.00 | 95.00 | C |
| ATOM | 3349 | CD | GLU | A | 221 | 12.697 | 10.962 | −4.627 | 1.00 | 103.69 | C |
| ATOM | 3350 | OE1 | GLU | A | 221 | 12.320 | 9.931 | −5.267 | 1.00 | 106.17 | O |
| ATOM | 3351 | OE2 | GLU | A | 221 | 12.071 | 12.079 | −4.702 | 1.00 | 106.53 | O |
| ATOM | 3352 | C | GLU | A | 221 | 15.116 | 7.478 | −2.181 | 1.00 | 86.16 | C |
| ATOM | 3353 | O | GLU | A | 221 | 16.103 | 7.484 | −1.457 | 1.00 | 82.16 | O |
| ATOM | 3354 | N | SER | A | 222 | 14.086 | 6.661 | −1.965 | 1.00 | 89.43 | N |
| ATOM | 3356 | CA | SER | A | 222 | 14.027 | 5.806 | −0.769 | 1.00 | 88.57 | C |
| ATOM | 3358 | CB | SER | A | 222 | 12.580 | 5.683 | −0.281 | 1.00 | 93.94 | C |
| ATOM | 3361 | OG | SER | A | 222 | 11.809 | 4.919 | −1.194 | 1.00 | 96.86 | O |
| ATOM | 3363 | C | SER | A | 222 | 14.640 | 4.395 | −0.949 | 1.00 | 85.65 | C |
| ATOM | 3364 | O | SER | A | 222 | 14.676 | 3.619 | 0.016 | 1.00 | 85.41 | O |
| ATOM | 3365 | N | ASP | A | 223 | 15.135 | 4.061 | −2.150 | 1.00 | 83.69 | N |
| ATOM | 3367 | CA | ASP | A | 223 | 15.657 | 2.711 | −2.417 | 1.00 | 80.96 | C |
| ATOM | 3369 | CB | ASP | A | 223 | 15.185 | 2.195 | −3.776 | 1.00 | 83.33 | C |
| ATOM | 3372 | CG | ASP | A | 223 | 13.703 | 2.299 | −3.944 | 1.00 | 89.14 | C |
| ATOM | 3373 | OD1 | ASP | A | 223 | 12.981 | 1.875 | −3.024 | 1.00 | 91.08 | O |
| ATOM | 3374 | OD2 | ASP | A | 223 | 13.165 | 2.800 | −4.951 | 1.00 | 92.92 | O |
| ATOM | 3375 | C | ASP | A | 223 | 17.171 | 2.672 | −2.346 | 1.00 | 75.27 | C |
| ATOM | 3376 | O | ASP | A | 223 | 17.825 | 1.737 | −2.855 | 1.00 | 73.21 | O |
| ATOM | 3377 | N | CYS | A | 224 | 17.746 | 3.656 | −1.683 | 1.00 | 72.71 | N |
| ATOM | 3379 | CA | CYS | A | 224 | 19.181 | 3.736 | −1.655 | 1.00 | 67.99 | C |
| ATOM | 3381 | CB | CYS | A | 224 | 19.598 | 5.080 | −1.135 | 1.00 | 67.67 | C |
| ATOM | 3384 | SG | CYS | A | 224 | 19.506 | 6.395 | −2.344 | 1.00 | 70.46 | S |
| ATOM | 3385 | C | CYS | A | 224 | 19.754 | 2.694 | −0.764 | 1.00 | 64.94 | C |
| ATOM | 3386 | O | CYS | A | 224 | 19.110 | 2.246 | 0.162 | 1.00 | 65.66 | O |
| ATOM | 3387 | N | LEU | A | 225 | 20.979 | 2.321 | −1.078 | 1.00 | 61.70 | N |
| ATOM | 3389 | CA | LEU | A | 225 | 21.782 | 1.447 | −0.245 | 1.00 | 59.61 | C |
| ATOM | 3391 | CB | LEU | A | 225 | 22.986 | 0.868 | −1.023 | 1.00 | 56.35 | C |
| ATOM | 3394 | CG | LEU | A | 225 | 22.685 | −0.402 | −1.891 | 1.00 | 56.31 | C |
| ATOM | 3396 | CD1 | LEU | A | 225 | 23.880 | −0.866 | −2.679 | 1.00 | 51.61 | C |
| ATOM | 3400 | CD2 | LEU | A | 225 | 22.126 | −1.570 | −1.021 | 1.00 | 56.37 | C |
| ATOM | 3404 | C | LEU | A | 225 | 22.260 | 2.296 | 0.878 | 1.00 | 59.46 | C |
| ATOM | 3405 | O | LEU | A | 225 | 22.097 | 1.964 | 2.032 | 1.00 | 60.50 | O |
| ATOM | 3406 | N | VAL | A | 226 | 22.876 | 3.410 | 0.501 | 1.00 | 59.46 | N |
| ATOM | 3408 | CA | VAL | A | 226 | 23.450 | 4.366 | 1.417 | 1.00 | 58.35 | C |
| ATOM | 3410 | CB | VAL | A | 226 | 25.010 | 4.197 | 1.616 | 1.00 | 54.93 | C |
| ATOM | 3412 | CG1 | VAL | A | 226 | 25.384 | 4.755 | 2.905 | 1.00 | 57.11 | C |
| ATOM | 3416 | CG2 | VAL | A | 226 | 25.449 | 2.804 | 1.665 | 1.00 | 54.18 | C |
| ATOM | 3420 | C | VAL | A | 226 | 23.165 | 5.729 | 0.779 | 1.00 | 59.60 | C |
| ATOM | 3421 | O | VAL | A | 226 | 22.958 | 5.810 | −0.407 | 1.00 | 60.42 | O |
| ATOM | 3422 | N | CYS | A | 227 | 23.138 | 6.793 | 1.574 | 1.00 | 60.27 | N |
| ATOM | 3424 | CA | CYS | A | 227 | 22.873 | 8.110 | 1.052 | 1.00 | 62.07 | C |
| ATOM | 3426 | CB | CYS | A | 227 | 22.091 | 8.926 | 2.031 | 1.00 | 64.54 | C |
| ATOM | 3429 | SG | CYS | A | 227 | 20.427 | 8.315 | 2.115 | 1.00 | 71.77 | S |
| ATOM | 3430 | C | CYS | A | 227 | 24.164 | 8.781 | 0.891 | 1.00 | 59.58 | C |
| ATOM | 3431 | O | CYS | A | 227 | 25.088 | 8.519 | 1.662 | 1.00 | 57.51 | O |
| ATOM | 3432 | N | ARG | A | 228 | 24.235 | 9.677 | −0.087 | 1.00 | 60.72 | N |
| ATOM | 3434 | CA | ARG | A | 228 | 25.465 | 10.401 | −0.289 | 1.00 | 58.88 | C |
| ATOM | 3436 | CB | ARG | A | 228 | 25.938 | 10.420 | −1.728 | 1.00 | 58.79 | C |
| ATOM | 3439 | CG | ARG | A | 228 | 25.066 | 10.857 | −2.770 | 1.00 | 60.60 | C |
| ATOM | 3442 | CD | ARG | A | 228 | 25.870 | 11.291 | −4.034 | 1.00 | 60.85 | C |
| ATOM | 3445 | NE | ARG | A | 228 | 25.464 | 12.688 | −4.273 | 1.00 | 66.58 | N |
| ATOM | 3447 | CZ | ARG | A | 228 | 25.007 | 13.147 | −5.391 | 1.00 | 68.99 | C |
| ATOM | 3448 | NH1 | ARG | A | 228 | 24.950 | 12.391 | −6.479 | 1.00 | 73.85 | N |
| ATOM | 3451 | NH2 | ARG | A | 228 | 24.612 | 14.369 | −5.438 | 1.00 | 73.63 | N |
| ATOM | 3454 | C | ARG | A | 228 | 25.487 | 11.753 | 0.315 | 1.00 | 60.04 | C |
| ATOM | 3455 | O | ARG | A | 228 | 26.542 | 12.340 | 0.407 | 1.00 | 59.64 | O |
| ATOM | 3456 | N | LYS | A | 229 | 24.352 | 12.222 | 0.791 | 1.00 | 62.48 | N |
| ATOM | 3458 | CA | LYS | A | 229 | 24.353 | 13.442 | 1.549 | 1.00 | 63.66 | C |
| ATOM | 3460 | CB | LYS | A | 229 | 23.648 | 14.573 | 0.792 | 1.00 | 67.05 | C |
| ATOM | 3463 | CG | LYS | A | 229 | 24.518 | 15.066 | −0.385 | 1.00 | 67.47 | C |
| ATOM | 3466 | CD | LYS | A | 229 | 23.831 | 16.061 | −1.381 | 1.00 | 73.70 | C |
| ATOM | 3469 | CE | LYS | A | 229 | 24.848 | 16.474 | −2.543 | 1.00 | 75.33 | C |
| ATOM | 3472 | NZ | LYS | A | 229 | 24.415 | 17.315 | −3.811 | 1.00 | 79.07 | N |
| ATOM | 3476 | C | LYS | A | 229 | 23.794 | 13.092 | 2.898 | 1.00 | 63.63 | C |
| ATOM | 3477 | O | LYS | A | 229 | 24.536 | 12.696 | 3.782 | 1.00 | 61.98 | O |
| ATOM | 3478 | N | PHE | A | 230 | 22.501 | 13.163 | 3.054 | 1.00 | 66.52 | N |
| ATOM | 3480 | CA | PHE | A | 230 | 21.906 | 12.856 | 4.332 | 1.00 | 68.08 | C |
| ATOM | 3482 | CB | PHE | A | 230 | 21.322 | 14.129 | 4.906 | 1.00 | 71.57 | C |
| ATOM | 3485 | CG | PHE | A | 230 | 22.344 | 15.070 | 5.540 | 1.00 | 72.46 | C |
| ATOM | 3486 | CD1 | PHE | A | 230 | 22.787 | 16.209 | 4.888 | 1.00 | 74.10 | C |
| ATOM | 3488 | CE1 | PHE | A | 230 | 23.692 | 17.117 | 5.502 | 1.00 | 74.49 | C |
| ATOM | 3490 | CZ | PHE | A | 230 | 24.146 | 16.887 | 6.773 | 1.00 | 74.16 | C |
| ATOM | 3492 | CE2 | PHE | A | 230 | 23.716 | 15.748 | 7.443 | 1.00 | 73.51 | C |
| ATOM | 3494 | CD2 | PHE | A | 230 | 22.800 | 14.859 | 6.831 | 1.00 | 74.05 | C |
| ATOM | 3496 | C | PHE | A | 230 | 20.816 | 11.770 | 4.136 | 1.00 | 69.44 | C |
| ATOM | 3497 | O | PHE | A | 230 | 20.160 | 11.708 | 3.069 | 1.00 | 71.17 | O |
| ATOM | 3498 | N | ARG | A | 231 | 20.676 | 10.891 | 5.130 | 1.00 | 68.61 | N |
| ATOM | 3500 | CA | ARG | A | 231 | 19.584 | 9.915 | 5.195 | 1.00 | 70.29 | C |
| ATOM | 3502 | CB | ARG | A | 231 | 20.074 | 8.526 | 5.708 | 1.00 | 67.41 | C |
| ATOM | 3511 | C | ARG | A | 231 | 18.552 | 10.519 | 6.134 | 1.00 | 74.06 | C |
| ATOM | 3512 | O | ARG | A | 231 | 18.922 | 11.069 | 7.149 | 1.00 | 73.47 | O |
| ATOM | 3513 | N | ASP | A | 232 | 17.274 | 10.431 | 5.798 | 1.00 | 78.37 | N |
| ATOM | 3515 | CA | ASP | A | 232 | 16.234 | 10.971 | 6.662 | 1.00 | 84.33 | C |
| ATOM | 3517 | CB | ASP | A | 232 | 15.144 | 11.728 | 5.891 | 1.00 | 88.64 | C |
| ATOM | 3520 | CG | ASP | A | 232 | 14.232 | 12.510 | 6.806 | 1.00 | 92.77 | C |
| ATOM | 3521 | OD1 | ASP | A | 232 | 14.285 | 12.270 | 8.018 | 1.00 | 92.46 | O |
| ATOM | 3522 | OD2 | ASP | A | 232 | 13.441 | 13.392 | 6.417 | 1.00 | 96.60 | O |
| ATOM | 3523 | C | ASP | A | 232 | 15.612 | 9.869 | 7.492 | 1.00 | 87.11 | C |
| ATOM | 3524 | O | ASP | A | 232 | 15.962 | 9.695 | 8.668 | 1.00 | 88.04 | O |
| ATOM | 3525 | N | GLU | A | 233 | 14.665 | 9.150 | 6.910 | 1.00 | 89.56 | N |
| ATOM | 3527 | CA | GLU | A | 233 | 13.976 | 8.117 | 7.627 | 1.00 | 91.75 | C |
| ATOM | 3529 | CB | GLU | A | 233 | 12.599 | 8.613 | 8.111 | 1.00 | 97.99 | C |
| ATOM | 3532 | CG | GLU | A | 233 | 12.519 | 8.786 | 9.622 | 1.00 | 100.66 | C |
| ATOM | 3535 | CD | GLU | A | 233 | 11.380 | 9.704 | 10.061 | 1.00 | 109.45 | C |
| ATOM | 3536 | OE1 | GLU | A | 233 | 10.502 | 9.995 | 9.208 | 1.00 | 113.90 | O |
| ATOM | 3537 | OE2 | GLU | A | 233 | 11.348 | 10.144 | 11.258 | 1.00 | 112.63 | O |
| ATOM | 3538 | C | GLU | A | 233 | 13.901 | 7.127 | 6.539 | 1.00 | 90.22 | C |
| ATOM | 3539 | O | GLU | A | 233 | 12.882 | 6.953 | 5.916 | 1.00 | 94.09 | O |
| ATOM | 3540 | N | ALA | A | 234 | 15.026 | 6.560 | 6.197 | 1.00 | 85.19 | N |
| ATOM | 3542 | CA | ALA | A | 234 | 15.025 | 5.743 | 4.982 | 1.00 | 84.82 | C |
| ATOM | 3544 | CB | ALA | A | 234 | 13.817 | 4.741 | 5.027 | 1.00 | 88.93 | C |
| ATOM | 3548 | C | ALA | A | 234 | 15.060 | 6.514 | 3.595 | 1.00 | 84.64 | C |
| ATOM | 3549 | O | ALA | A | 234 | 15.340 | 5.900 | 2.543 | 1.00 | 83.37 | O |
| ATOM | 3550 | N | THR | A | 235 | 14.790 | 7.827 | 3.601 | 1.00 | 85.96 | N |
| ATOM | 3552 | CA | THR | A | 235 | 14.869 | 8.675 | 2.402 | 1.00 | 85.56 | C |
| ATOM | 3554 | CB | THR | A | 235 | 13.751 | 9.674 | 2.489 | 1.00 | 90.41 | C |
| ATOM | 3556 | OG1 | THR | A | 235 | 12.551 | 8.977 | 2.820 | 1.00 | 94.00 | O |
| ATOM | 3558 | CG2 | THR | A | 235 | 13.455 | 10.298 | 1.155 | 1.00 | 91.65 | C |
| ATOM | 3562 | C | THR | A | 235 | 16.208 | 9.413 | 2.264 | 1.00 | 81.37 | C |
| ATOM | 3563 | O | THR | A | 235 | 16.840 | 9.680 | 3.237 | 1.00 | 80.40 | O |
| ATOM | 3564 | N | CYS | A | 236 | 16.631 | 9.764 | 1.059 | 1.00 | 80.26 | N |
| ATOM | 3566 | CA | CYS | A | 236 | 17.911 | 10.456 | 0.845 | 1.00 | 76.60 | C |
| ATOM | 3568 | CB | CYS | A | 236 | 18.705 | 9.695 | −0.191 | 1.00 | 73.69 | C |
| ATOM | 3571 | SG | CYS | A | 236 | 19.452 | 8.145 | 0.356 | 1.00 | 71.68 | S |
| ATOM | 3572 | C | CYS | A | 236 | 17.771 | 11.919 | 0.358 | 1.00 | 78.31 | C |
| ATOM | 3573 | O | CYS | A | 236 | 17.255 | 12.169 | −0.717 | 1.00 | 80.49 | O |
| ATOM | 3574 | N | LYS | A | 237 | 18.295 | 12.873 | 1.114 | 1.00 | 77.15 | N |
| ATOM | 3576 | CA | LYS | A | 237 | 18.017 | 14.294 | 0.879 | 1.00 | 79.48 | C |
| ATOM | 3578 | CB | LYS | A | 237 | 17.178 | 14.842 | 2.038 | 1.00 | 82.43 | C |
| ATOM | 3581 | CG | LYS | A | 237 | 16.038 | 13.951 | 2.407 | 1.00 | 84.83 | C |
| ATOM | 3584 | CD | LYS | A | 237 | 15.070 | 14.580 | 3.357 | 1.00 | 90.16 | C |
| ATOM | 3587 | CE | LYS | A | 237 | 13.746 | 13.819 | 3.358 | 1.00 | 94.88 | C |
| ATOM | 3590 | NZ | LYS | A | 237 | 12.689 | 14.539 | 4.103 | 1.00 | 102.61 | N |
| ATOM | 3594 | C | LYS | A | 237 | 19.292 | 15.106 | 0.759 | 1.00 | 76.46 | C |
| ATOM | 3595 | O | LYS | A | 237 | 20.312 | 14.741 | 1.276 | 1.00 | 72.36 | O |
| ATOM | 3596 | N | ASP | A | 238 | 19.216 | 16.234 | 0.079 | 1.00 | 78.98 | N |
| ATOM | 3598 | CA | ASP | A | 238 | 20.321 | 17.193 | 0.047 | 1.00 | 77.64 | C |
| ATOM | 3600 | CB | ASP | A | 238 | 19.975 | 18.348 | −0.895 | 1.00 | 81.85 | C |
| ATOM | 3603 | CG | ASP | A | 238 | 19.772 | 17.891 | −2.338 | 1.00 | 82.50 | C |
| ATOM | 3604 | OD1 | ASP | A | 238 | 20.406 | 16.888 | −2.690 | 1.00 | 80.05 | O |
| ATOM | 3605 | OD2 | ASP | A | 238 | 19.015 | 18.454 | −3.170 | 1.00 | 84.38 | O |
| ATOM | 3606 | C | ASP | A | 238 | 20.641 | 17.763 | 1.424 | 1.00 | 76.62 | C |
| ATOM | 3607 | O | ASP | A | 238 | 21.770 | 18.114 | 1.707 | 1.00 | 73.74 | O |
| ATOM | 3608 | N | THR | A | 239 | 19.645 | 17.833 | 2.290 | 1.00 | 79.33 | N |
| ATOM | 3610 | CA | THR | A | 239 | 19.820 | 18.465 | 3.586 | 1.00 | 80.08 | C |
| ATOM | 3612 | CB | THR | A | 239 | 19.799 | 20.008 | 3.363 | 1.00 | 84.18 | C |
| ATOM | 3614 | OG1 | THR | A | 239 | 20.373 | 20.685 | 4.484 | 1.00 | 83.41 | O |
| ATOM | 3616 | CG2 | THR | A | 239 | 18.371 | 20.544 | 3.222 | 1.00 | 88.96 | C |
| ATOM | 3620 | C | THR | A | 239 | 18.769 | 18.064 | 4.591 | 1.00 | 82.04 | C |
| ATOM | 3621 | O | THR | A | 239 | 17.690 | 17.627 | 4.250 | 1.00 | 84.18 | O |
| ATOM | 3622 | N | CYS | A | 240 | 19.076 | 18.261 | 5.847 | 1.00 | 82.76 | N |
| ATOM | 3624 | CA | CYS | A | 240 | 18.073 | 18.046 | 6.850 | 1.00 | 87.41 | C |
| ATOM | 3626 | CB | CYS | A | 240 | 18.686 | 17.977 | 8.227 | 1.00 | 85.89 | C |
| ATOM | 3629 | SG | CYS | A | 240 | 19.820 | 16.589 | 8.264 | 1.00 | 84.87 | S |
| ATOM | 3630 | C | CYS | A | 240 | 17.039 | 19.132 | 6.784 | 1.00 | 93.59 | C |
| ATOM | 3631 | O | CYS | A | 240 | 17.356 | 20.313 | 6.683 | 1.00 | 95.73 | O |
| ATOM | 3632 | N | PRO | A | 241 | 15.780 | 18.740 | 6.812 | 1.00 | 55.04 | N |
| ATOM | 3633 | CA | PRO | A | 241 | 14.682 | 19.707 | 6.985 | 1.00 | 56.88 | C |
| ATOM | 3635 | CB | PRO | A | 241 | 13.431 | 18.836 | 7.235 | 1.00 | 58.19 | C |
| ATOM | 3638 | CG | PRO | A | 241 | 13.919 | 17.393 | 7.321 | 1.00 | 57.06 | C |
| ATOM | 3641 | CD | PRO | A | 241 | 15.281 | 17.357 | 6.687 | 1.00 | 54.91 | C |
| ATOM | 3644 | C | PRO | A | 241 | 14.936 | 20.540 | 8.198 | 1.00 | 57.83 | C |
| ATOM | 3645 | O | PRO | A | 241 | 15.201 | 20.028 | 9.281 | 1.00 | 57.58 | O |
| ATOM | 3646 | N | PRO | A | 242 | 14.852 | 21.827 | 8.038 | 1.00 | 58.40 | N |
| ATOM | 3647 | CA | PRO | A | 242 | 15.392 | 22.690 | 9.071 | 1.00 | 59.18 | C |
| ATOM | 3649 | CB | PRO | A | 242 | 15.812 | 23.929 | 8.325 | 1.00 | 59.59 | C |
| ATOM | 3652 | CG | PRO | A | 242 | 15.087 | 23.771 | 6.931 | 1.00 | 60.42 | C |
| ATOM | 3655 | CD | PRO | A | 242 | 14.198 | 22.555 | 6.961 | 1.00 | 58.64 | C |
| ATOM | 3658 | C | PRO | A | 242 | 14.349 | 22.986 | 10.097 | 1.00 | 60.49 | C |
| ATOM | 3659 | O | PRO | A | 242 | 13.169 | 22.609 | 9.956 | 1.00 | 58.83 | O |
| ATOM | 3660 | N | LEU | A | 243 | 14.859 | 23.681 | 11.113 | 1.00 | 61.42 | N |
| ATOM | 3662 | CA | LEU | A | 243 | 14.198 | 23.925 | 12.387 | 1.00 | 63.21 | C |
| ATOM | 3664 | CB | LEU | A | 243 | 15.253 | 24.301 | 13.402 | 1.00 | 63.51 | C |
| ATOM | 3667 | CG | LEU | A | 243 | 16.030 | 23.016 | 13.747 | 1.00 | 64.54 | C |
| ATOM | 3669 | CD1 | LEU | A | 243 | 17.404 | 23.380 | 14.430 | 1.00 | 64.32 | C |
| ATOM | 3673 | CD2 | LEU | A | 243 | 15.119 | 21.989 | 14.577 | 1.00 | 64.65 | C |
| ATOM | 3677 | C | LEU | A | 243 | 13.212 | 25.037 | 12.331 | 1.00 | 64.28 | C |
| ATOM | 3678 | O | LEU | A | 243 | 12.271 | 25.089 | 13.119 | 1.00 | 65.42 | O |
| ATOM | 3679 | N | MET | A | 244 | 13.425 | 25.919 | 11.372 | 1.00 | 63.70 | N |
| ATOM | 3681 | CA | MET | A | 244 | 12.606 | 27.066 | 11.251 | 1.00 | 65.18 | C |
| ATOM | 3683 | CB | MET | A | 244 | 13.312 | 28.151 | 11.980 | 1.00 | 65.81 | C |
| ATOM | 3686 | CG | MET | A | 244 | 13.551 | 27.784 | 13.412 | 1.00 | 66.25 | C |
| ATOM | 3689 | SD | MET | A | 244 | 13.078 | 29.142 | 14.347 | 1.00 | 72.32 | S |
| ATOM | 3690 | CE | MET | A | 244 | 11.731 | 28.403 | 15.295 | 1.00 | 73.17 | C |
| ATOM | 3694 | C | MET | A | 244 | 12.399 | 27.474 | 9.833 | 1.00 | 65.08 | C |
| ATOM | 3695 | O | MET | A | 244 | 13.312 | 27.447 | 9.054 | 1.00 | 63.58 | O |
| ATOM | 3696 | N | LEU | A | 245 | 11.177 | 27.876 | 9.529 | 1.00 | 67.08 | N |
| ATOM | 3698 | CA | LEU | A | 245 | 10.803 | 28.355 | 8.241 | 1.00 | 67.76 | C |
| ATOM | 3700 | CB | LEU | A | 245 | 9.739 | 27.435 | 7.703 | 1.00 | 68.55 | C |
| ATOM | 3703 | CG | LEU | A | 245 | 10.253 | 26.021 | 7.432 | 1.00 | 67.01 | C |
| ATOM | 3705 | CD1 | LEU | A | 245 | 9.067 | 25.110 | 7.212 | 1.00 | 68.07 | C |
| ATOM | 3709 | CD2 | LEU | A | 245 | 11.186 | 25.963 | 6.238 | 1.00 | 65.23 | C |
| ATOM | 3713 | C | LEU | A | 245 | 10.185 | 29.719 | 8.344 | 1.00 | 70.42 | C |
| ATOM | 3714 | O | LEU | A | 245 | 9.435 | 29.957 | 9.281 | 1.00 | 71.97 | O |
| ATOM | 3715 | N | TYR | A | 246 | 10.457 | 30.590 | 7.355 | 1.00 | 70.54 | N |
| ATOM | 3717 | CA | TYR | A | 246 | 9.619 | 31.746 | 7.100 | 1.00 | 72.93 | C |
| ATOM | 3719 | CB | TYR | A | 246 | 10.028 | 32.498 | 5.833 | 1.00 | 72.99 | C |
| ATOM | 3722 | CG | TYR | A | 246 | 9.424 | 33.903 | 5.785 | 1.00 | 76.04 | C |
| ATOM | 3723 | CD1 | TYR | A | 246 | 10.123 | 34.980 | 6.284 | 1.00 | 76.12 | C |
| ATOM | 3725 | CE1 | TYR | A | 246 | 9.595 | 36.220 | 6.294 | 1.00 | 78.89 | C |
| ATOM | 3727 | CZ | TYR | A | 246 | 8.358 | 36.417 | 5.810 | 1.00 | 80.72 | C |
| ATOM | 3728 | OH | TYR | A | 246 | 7.871 | 37.715 | 5.849 | 1.00 | 84.16 | O |
| ATOM | 3730 | CE2 | TYR | A | 246 | 7.616 | 35.344 | 5.325 | 1.00 | 79.96 | C |
| ATOM | 3732 | CD2 | TYR | A | 246 | 8.134 | 34.126 | 5.310 | 1.00 | 76.25 | C |
| ATOM | 3734 | C | TYR | A | 246 | 8.167 | 31.318 | 6.950 | 1.00 | 75.65 | C |
| ATOM | 3735 | O | TYR | A | 246 | 7.842 | 30.513 | 6.113 | 1.00 | 75.18 | O |
| ATOM | 3736 | N | ASN | A | 247 | 7.289 | 31.893 | 7.749 | 1.00 | 79.06 | N |
| ATOM | 3738 | CA | ASN | A | 247 | 5.865 | 31.606 | 7.710 | 1.00 | 81.97 | C |
| ATOM | 3740 | CB | ASN | A | 247 | 5.369 | 31.485 | 9.144 | 1.00 | 83.33 | C |
| ATOM | 3743 | CG | ASN | A | 247 | 4.117 | 30.711 | 9.248 | 1.00 | 86.35 | C |
| ATOM | 3744 | OD1 | ASN | A | 247 | 3.048 | 31.184 | 8.805 | 1.00 | 90.91 | O |
| ATOM | 3745 | ND2 | ASN | A | 247 | 4.212 | 29.494 | 9.824 | 1.00 | 83.97 | N |
| ATOM | 3748 | C | ASN | A | 247 | 5.152 | 32.746 | 7.008 | 1.00 | 84.93 | C |
| ATOM | 3749 | O | ASN | A | 247 | 5.206 | 33.878 | 7.458 | 1.00 | 85.90 | O |
| ATOM | 3750 | N | PRO | A | 248 | 4.500 | 32.451 | 5.903 | 1.00 | 86.39 | N |
| ATOM | 3751 | CA | PRO | A | 248 | 3.782 | 33.478 | 5.096 | 1.00 | 90.09 | C |
| ATOM | 3753 | CB | PRO | A | 248 | 3.144 | 32.675 | 3.968 | 1.00 | 90.03 | C |
| ATOM | 3756 | CG | PRO | A | 248 | 3.918 | 31.316 | 3.957 | 1.00 | 86.36 | C |
| ATOM | 3759 | CD | PRO | A | 248 | 4.423 | 31.092 | 5.346 | 1.00 | 84.43 | C |
| ATOM | 3762 | C | PRO | A | 248 | 2.673 | 34.251 | 5.837 | 1.00 | 94.90 | C |
| ATOM | 3763 | O | PRO | A | 248 | 2.511 | 35.494 | 5.688 | 1.00 | 96.88 | O |
| ATOM | 3764 | N | THR | A | 249 | 1.905 | 33.491 | 6.619 | 1.00 | 96.37 | N |
| ATOM | 3766 | CA | THR | A | 249 | 0.842 | 34.054 | 7.446 | 1.00 | 101.00 | C |
| ATOM | 3768 | CB | THR | A | 249 | −0.184 | 32.930 | 7.919 | 1.00 | 102.23 | C |
| ATOM | 3770 | OG1 | THR | A | 249 | −1.321 | 32.895 | 7.044 | 1.00 | 104.68 | O |
| ATOM | 3772 | CG2 | THR | A | 249 | −0.848 | 33.250 | 9.282 | 1.00 | 105.46 | C |
| ATOM | 3776 | C | THR | A | 249 | 1.457 | 34.842 | 8.633 | 1.00 | 100.86 | C |
| ATOM | 3777 | O | THR | A | 249 | 1.021 | 35.972 | 8.921 | 1.00 | 104.58 | O |
| ATOM | 3778 | N | THR | A | 250 | 2.463 | 34.262 | 9.304 | 1.00 | 96.81 | N |
| ATOM | 3780 | CA | THR | A | 250 | 3.064 | 34.893 | 10.490 | 1.00 | 95.38 | C |
| ATOM | 3782 | CB | THR | A | 250 | 3.973 | 33.896 | 11.305 | 1.00 | 91.35 | C |
| ATOM | 3784 | OG1 | THR | A | 250 | 3.395 | 32.584 | 11.401 | 1.00 | 90.19 | O |
| ATOM | 3786 | CG2 | THR | A | 250 | 4.032 | 34.315 | 12.723 | 1.00 | 91.91 | C |
| ATOM | 3790 | C | THR | A | 250 | 3.893 | 36.124 | 10.108 | 1.00 | 94.90 | C |
| ATOM | 3791 | O | THR | A | 250 | 4.075 | 37.028 | 10.936 | 1.00 | 96.04 | O |
| ATOM | 3792 | N | TYR | A | 251 | 4.380 | 36.156 | 8.861 | 1.00 | 93.34 | N |
| ATOM | 3794 | CA | TYR | A | 251 | 5.414 | 37.103 | 8.412 | 1.00 | 92.01 | C |
| ATOM | 3796 | CB | TYR | A | 251 | 4.905 | 38.559 | 8.423 | 1.00 | 95.71 | C |
| ATOM | 3799 | CG | TYR | A | 251 | 3.736 | 38.867 | 7.485 | 1.00 | 100.54 | C |
| ATOM | 3800 | CD1 | TYR | A | 251 | 2.415 | 38.810 | 7.927 | 1.00 | 103.41 | C |
| ATOM | 3802 | CE1 | TYR | A | 251 | 1.387 | 39.090 | 7.079 | 1.00 | 106.88 | C |
| ATOM | 3804 | CZ | TYR | A | 251 | 1.655 | 39.462 | 5.781 | 1.00 | 106.57 | C |
| ATOM | 3805 | OH | TYR | A | 251 | 0.633 | 39.762 | 4.918 | 1.00 | 110.34 | O |
| ATOM | 3807 | CE2 | TYR | A | 251 | 2.937 | 39.552 | 5.333 | 1.00 | 102.86 | C |
| ATOM | 3809 | CD2 | TYR | A | 251 | 3.962 | 39.262 | 6.165 | 1.00 | 100.61 | C |
| ATOM | 3811 | C | TYR | A | 251 | 6.699 | 36.995 | 9.259 | 1.00 | 88.23 | C |
| ATOM | 3812 | O | TYR | A | 251 | 7.362 | 37.985 | 9.536 | 1.00 | 88.44 | O |
| ATOM | 3813 | N | GLN | A | 252 | 7.059 | 35.806 | 9.697 | 1.00 | 84.91 | N |
| ATOM | 3815 | CA | GLN | A | 252 | 8.325 | 35.701 | 10.397 | 1.00 | 81.92 | C |
| ATOM | 3817 | CB | GLN | A | 252 | 8.134 | 36.158 | 11.840 | 1.00 | 82.92 | C |
| ATOM | 3820 | CG | GLN | A | 252 | 7.289 | 35.204 | 12.679 | 1.00 | 84.45 | C |
| ATOM | 3823 | CD | GLN | A | 252 | 6.830 | 35.822 | 13.980 | 1.00 | 88.01 | C |
| ATOM | 3824 | OE1 | GLN | A | 252 | 7.570 | 36.606 | 14.587 | 1.00 | 89.53 | O |
| ATOM | 3825 | NE2 | GLN | A | 252 | 5.603 | 35.504 | 14.406 | 1.00 | 89.99 | N |
| ATOM | 3828 | C | GLN | A | 252 | 8.961 | 34.304 | 10.327 | 1.00 | 78.33 | C |
| ATOM | 3829 | O | GLN | A | 252 | 8.474 | 33.408 | 9.676 | 1.00 | 76.85 | O |
| ATOM | 3830 | N | MET | A | 253 | 10.095 | 34.153 | 10.987 | 1.00 | 76.53 | N |
| ATOM | 3832 | CA | MET | A | 253 | 10.666 | 32.843 | 11.258 | 1.00 | 74.03 | C |
| ATOM | 3834 | CB | MET | A | 253 | 12.137 | 32.999 | 11.601 | 1.00 | 71.87 | C |
| ATOM | 3837 | CG | MET | A | 253 | 12.931 | 33.489 | 10.435 | 1.00 | 71.14 | C |
| ATOM | 3840 | SD | MET | A | 253 | 12.878 | 32.239 | 9.059 | 1.00 | 75.45 | S |
| ATOM | 3841 | CE | MET | A | 253 | 12.407 | 33.204 | 7.938 | 1.00 | 75.10 | C |
| ATOM | 3845 | C | MET | A | 253 | 9.874 | 32.178 | 12.387 | 1.00 | 75.06 | C |
| ATOM | 3846 | O | MET | A | 253 | 9.697 | 32.729 | 13.464 | 1.00 | 76.02 | O |
| ATOM | 3847 | N | ASP | A | 254 | 9.369 | 30.994 | 12.098 | 1.00 | 75.32 | N |
| ATOM | 3849 | CA | ASP | A | 254 | 8.560 | 30.212 | 13.023 | 1.00 | 76.65 | C |
| ATOM | 3851 | CB | ASP | A | 254 | 7.086 | 30.304 | 12.587 | 1.00 | 79.52 | C |
| ATOM | 3854 | CG | ASP | A | 254 | 6.174 | 30.884 | 13.663 | 1.00 | 82.89 | C |
| ATOM | 3855 | OD1 | ASP | A | 254 | 6.563 | 30.843 | 14.866 | 1.00 | 82.32 | O |
| ATOM | 3856 | OD2 | ASP | A | 254 | 5.037 | 31.370 | 13.376 | 1.00 | 86.13 | O |
| ATOM | 3857 | C | ASP | A | 254 | 9.022 | 28.724 | 13.021 | 1.00 | 74.63 | C |
| ATOM | 3858 | O | ASP | A | 254 | 9.382 | 28.192 | 11.985 | 1.00 | 73.66 | O |
| ATOM | 3859 | N | VAL | A | 255 | 8.988 | 28.059 | 14.174 | 1.00 | 74.49 | N |
| ATOM | 3861 | CA | VAL | A | 255 | 9.301 | 26.612 | 14.277 | 1.00 | 72.81 | C |
| ATOM | 3863 | CB | VAL | A | 255 | 8.787 | 25.976 | 15.627 | 1.00 | 73.73 | C |
| ATOM | 3865 | CG1 | VAL | A | 255 | 9.722 | 24.890 | 16.070 | 1.00 | 72.16 | C |
| ATOM | 3869 | CG2 | VAL | A | 255 | 8.622 | 27.020 | 16.785 | 1.00 | 76.62 | C |
| ATOM | 3873 | C | VAL | A | 255 | 8.707 | 25.762 | 13.148 | 1.00 | 72.23 | C |
| ATOM | 3874 | O | VAL | A | 255 | 7.497 | 25.708 | 12.977 | 1.00 | 73.53 | O |
| ATOM | 3875 | N | ASN | A | 256 | 9.566 | 25.088 | 12.389 | 1.00 | 70.27 | N |
| ATOM | 3877 | CA | ASN | A | 256 | 9.101 | 24.122 | 11.380 | 1.00 | 70.31 | C |
| ATOM | 3879 | CB | ASN | A | 256 | 10.182 | 23.791 | 10.348 | 1.00 | 67.62 | C |
| ATOM | 3882 | CG | ASN | A | 256 | 9.858 | 22.537 | 9.502 | 1.00 | 65.90 | C |
| ATOM | 3883 | OD1 | ASN | A | 256 | 8.855 | 21.859 | 9.709 | 1.00 | 65.23 | O |
| ATOM | 3884 | ND2 | ASN | A | 256 | 10.740 | 22.225 | 8.558 | 1.00 | 63.09 | N |
| ATOM | 3887 | C | ASN | A | 256 | 8.719 | 22.844 | 12.077 | 1.00 | 71.03 | C |
| ATOM | 3888 | O | ASN | A | 256 | 9.570 | 22.241 | 12.710 | 1.00 | 68.99 | O |
| ATOM | 3889 | N | PRO | A | 257 | 7.457 | 22.424 | 11.954 | 1.00 | 74.05 | N |
| ATOM | 3890 | CA | PRO | A | 257 | 7.007 | 21.212 | 12.660 | 1.00 | 74.85 | C |
| ATOM | 3892 | CB | PRO | A | 257 | 5.467 | 21.181 | 12.420 | 1.00 | 77.52 | C |
| ATOM | 3895 | CG | PRO | A | 257 | 5.247 | 21.916 | 11.101 | 1.00 | 78.04 | C |
| ATOM | 3898 | CD | PRO | A | 257 | 6.380 | 23.014 | 11.114 | 1.00 | 76.18 | C |
| ATOM | 3901 | C | PRO | A | 257 | 7.711 | 19.971 | 12.080 | 1.00 | 72.67 | C |
| ATOM | 3902 | O | PRO | A | 257 | 8.226 | 19.202 | 12.894 | 1.00 | 73.12 | O |
| ATOM | 3903 | N | GLU | A | 258 | 7.762 | 19.793 | 10.752 | 1.00 | 71.09 | N |
| ATOM | 3905 | CA | GLU | A | 258 | 8.441 | 18.626 | 10.148 | 1.00 | 68.98 | C |
| ATOM | 3907 | CB | GLU | A | 258 | 8.100 | 18.532 | 8.631 | 1.00 | 68.15 | C |
| ATOM | 3914 | C | GLU | A | 258 | 9.997 | 18.534 | 10.399 | 1.00 | 66.74 | C |
| ATOM | 3915 | O | GLU | A | 258 | 10.638 | 17.563 | 9.979 | 1.00 | 65.05 | O |
| ATOM | 3916 | N | GLY | A | 259 | 10.584 | 19.518 | 11.105 | 1.00 | 66.61 | N |
| ATOM | 3918 | CA | GLY | A | 259 | 12.031 | 19.749 | 11.122 | 1.00 | 64.31 | C |
| ATOM | 3921 | C | GLY | A | 259 | 12.826 | 18.787 | 11.970 | 1.00 | 63.46 | C |
| ATOM | 3922 | O | GLY | A | 259 | 12.237 | 18.134 | 12.863 | 1.00 | 64.80 | O |
| ATOM | 3923 | N | LYS | A | 260 | 14.148 | 18.732 | 11.713 | 1.00 | 61.22 | N |
| ATOM | 3925 | CA | LYS | A | 260 | 15.061 | 17.684 | 12.239 | 1.00 | 59.87 | C |
| ATOM | 3927 | CB | LYS | A | 260 | 15.012 | 16.435 | 11.361 | 1.00 | 58.96 | C |
| ATOM | 3930 | CG | LYS | A | 260 | 13.635 | 15.850 | 11.292 | 1.00 | 61.80 | C |
| ATOM | 3933 | CD | LYS | A | 260 | 13.592 | 14.414 | 10.821 | 1.00 | 63.51 | C |
| ATOM | 3936 | CE | LYS | A | 260 | 12.464 | 14.188 | 9.801 | 1.00 | 64.20 | C |
| ATOM | 3939 | NZ | LYS | A | 260 | 11.982 | 12.820 | 9.961 | 1.00 | 68.50 | N |
| ATOM | 3943 | C | LYS | A | 260 | 16.528 | 18.103 | 12.418 | 1.00 | 57.61 | C |
| ATOM | 3944 | O | LYS | A | 260 | 17.017 | 18.961 | 11.792 | 1.00 | 57.42 | O |
| ATOM | 3945 | N | TYR | A | 261 | 17.211 | 17.436 | 13.305 | 1.00 | 56.94 | N |
| ATOM | 3947 | CA | TYR | A | 261 | 18.543 | 17.762 | 13.716 | 1.00 | 55.87 | C |
| ATOM | 3949 | CB | TYR | A | 261 | 18.663 | 17.429 | 15.213 | 1.00 | 56.86 | C |
| ATOM | 3952 | CG | TYR | A | 261 | 17.714 | 18.203 | 16.097 | 1.00 | 58.81 | C |
| ATOM | 3953 | CD1 | TYR | A | 261 | 16.747 | 17.586 | 16.878 | 1.00 | 62.63 | C |
| ATOM | 3955 | CE1 | TYR | A | 261 | 15.864 | 18.338 | 17.722 | 1.00 | 63.21 | C |
| ATOM | 3957 | CZ | TYR | A | 261 | 15.972 | 19.694 | 17.733 | 1.00 | 64.10 | C |
| ATOM | 3958 | OH | TYR | A | 261 | 15.188 | 20.526 | 18.498 | 1.00 | 67.69 | O |
| ATOM | 3960 | CE2 | TYR | A | 261 | 16.924 | 20.311 | 16.958 | 1.00 | 64.45 | C |
| ATOM | 3962 | CD2 | TYR | A | 261 | 17.785 | 19.570 | 16.154 | 1.00 | 61.93 | C |
| ATOM | 3964 | C | TYR | A | 261 | 19.496 | 16.898 | 12.904 | 1.00 | 54.12 | C |
| ATOM | 3965 | O | TYR | A | 261 | 19.153 | 15.858 | 12.422 | 1.00 | 53.80 | O |
| ATOM | 3966 | N | SER | A | 262 | 20.729 | 17.298 | 12.811 | 1.00 | 53.84 | N |
| ATOM | 3968 | CA | SER | A | 262 | 21.669 | 16.598 | 12.024 | 1.00 | 53.32 | C |
| ATOM | 3970 | CB | SER | A | 262 | 22.452 | 17.594 | 11.178 | 1.00 | 52.59 | C |
| ATOM | 3973 | OG | SER | A | 262 | 23.776 | 17.761 | 11.661 | 1.00 | 53.41 | O |
| ATOM | 3975 | C | SER | A | 262 | 22.566 | 15.727 | 12.939 | 1.00 | 54.56 | C |
| ATOM | 3976 | O | SER | A | 262 | 23.248 | 16.220 | 13.815 | 1.00 | 55.50 | O |
| ATOM | 3977 | N | PHE | A | 263 | 22.561 | 14.423 | 12.719 | 1.00 | 55.44 | N |
| ATOM | 3979 | CA | PHE | A | 263 | 23.483 | 13.544 | 13.407 | 1.00 | 58.17 | C |
| ATOM | 3981 | CB | PHE | A | 263 | 22.733 | 12.590 | 14.327 | 1.00 | 59.86 | C |
| ATOM | 3984 | CG | PHE | A | 263 | 23.613 | 11.673 | 15.142 | 1.00 | 61.87 | C |
| ATOM | 3985 | CD1 | PHE | A | 263 | 24.705 | 12.159 | 15.826 | 1.00 | 62.54 | C |
| ATOM | 3987 | CE1 | PHE | A | 263 | 25.441 | 11.352 | 16.570 | 1.00 | 64.60 | C |
| ATOM | 3989 | CZ | PHE | A | 263 | 25.119 | 10.015 | 16.657 | 1.00 | 67.48 | C |
| ATOM | 3991 | CE2 | PHE | A | 263 | 24.039 | 9.515 | 16.003 | 1.00 | 65.99 | C |
| ATOM | 3993 | CD2 | PHE | A | 263 | 23.287 | 10.334 | 15.264 | 1.00 | 63.61 | C |
| ATOM | 3995 | C | PHE | A | 263 | 24.214 | 12.764 | 12.367 | 1.00 | 58.42 | C |
| ATOM | 3996 | O | PHE | A | 263 | 23.644 | 11.903 | 11.736 | 1.00 | 57.38 | O |
| ATOM | 3997 | N | GLY | A | 264 | 25.513 | 13.051 | 12.319 | 1.00 | 60.11 | N |
| ATOM | 3999 | CA | GLY | A | 264 | 26.439 | 12.853 | 11.228 | 1.00 | 59.81 | C |
| ATOM | 4002 | C | GLY | A | 264 | 25.990 | 12.114 | 10.022 | 1.00 | 58.59 | C |
| ATOM | 4003 | O | GLY | A | 264 | 26.208 | 10.919 | 9.926 | 1.00 | 61.26 | O |
| ATOM | 4004 | N | ALA | A | 265 | 25.426 | 12.803 | 9.068 | 1.00 | 56.84 | N |
| ATOM | 4006 | CA | ALA | A | 265 | 24.939 | 12.139 | 7.818 | 1.00 | 55.65 | C |
| ATOM | 4008 | CB | ALA | A | 265 | 25.948 | 11.151 | 7.134 | 1.00 | 55.54 | C |
| ATOM | 4012 | C | ALA | A | 265 | 23.642 | 11.468 | 8.026 | 1.00 | 54.37 | C |
| ATOM | 4013 | O | ALA | A | 265 | 23.287 | 10.620 | 7.246 | 1.00 | 54.14 | O |
| ATOM | 4014 | N | THR | A | 266 | 22.924 | 11.936 | 9.029 | 1.00 | 54.84 | N |
| ATOM | 4016 | CA | THR | A | 266 | 21.622 | 11.451 | 9.350 | 1.00 | 54.78 | C |
| ATOM | 4018 | CB | THR | A | 266 | 21.814 | 10.371 | 10.300 | 1.00 | 56.35 | C |
| ATOM | 4020 | OG1 | THR | A | 266 | 21.628 | 9.203 | 9.556 | 1.00 | 55.31 | O |
| ATOM | 4022 | CG2 | THR | A | 266 | 20.782 | 10.293 | 11.424 | 1.00 | 58.51 | C |
| ATOM | 4026 | C | THR | A | 266 | 20.750 | 12.549 | 9.891 | 1.00 | 55.45 | C |
| ATOM | 4027 | O | THR | A | 266 | 21.247 | 13.566 | 10.359 | 1.00 | 55.61 | O |
| ATOM | 4028 | N | CYS | A | 267 | 19.445 | 12.342 | 9.755 | 1.00 | 55.64 | N |
| ATOM | 4030 | CA | CYS | A | 267 | 18.432 | 13.347 | 10.049 | 1.00 | 56.01 | C |
| ATOM | 4032 | CB | CYS | A | 267 | 17.598 | 13.692 | 8.831 | 1.00 | 54.41 | C |
| ATOM | 4035 | SG | CYS | A | 267 | 18.513 | 14.786 | 7.754 | 1.00 | 52.64 | S |
| ATOM | 4036 | C | CYS | A | 267 | 17.547 | 12.698 | 11.016 | 1.00 | 57.94 | C |
| ATOM | 4037 | O | CYS | A | 267 | 17.059 | 11.627 | 10.716 | 1.00 | 58.45 | O |
| ATOM | 4038 | N | VAL | A | 268 | 17.273 | 13.378 | 12.130 | 1.00 | 59.39 | N |
| ATOM | 4040 | CA | VAL | A | 268 | 16.860 | 12.719 | 13.344 | 1.00 | 61.02 | C |
| ATOM | 4042 | CB | VAL | A | 268 | 18.164 | 12.290 | 14.042 | 1.00 | 61.46 | C |
| ATOM | 4044 | CG1 | VAL | A | 268 | 18.230 | 12.613 | 15.536 | 1.00 | 61.61 | C |
| ATOM | 4048 | CG2 | VAL | A | 268 | 18.399 | 10.822 | 13.748 | 1.00 | 62.00 | C |
| ATOM | 4052 | C | VAL | A | 268 | 16.000 | 13.647 | 14.197 | 1.00 | 62.66 | C |
| ATOM | 4053 | O | VAL | A | 268 | 16.450 | 14.752 | 14.458 | 1.00 | 62.80 | O |
| ATOM | 4054 | N | LYS | A | 269 | 14.799 | 13.212 | 14.646 | 1.00 | 63.83 | N |
| ATOM | 4056 | CA | LYS | A | 269 | 13.909 | 14.122 | 15.410 | 1.00 | 65.02 | C |
| ATOM | 4058 | CB | LYS | A | 269 | 12.413 | 13.700 | 15.399 | 1.00 | 65.91 | C |
| ATOM | 4065 | C | LYS | A | 269 | 14.398 | 14.338 | 16.842 | 1.00 | 65.84 | C |
| ATOM | 4066 | O | LYS | A | 269 | 14.126 | 15.373 | 17.438 | 1.00 | 65.90 | O |
| ATOM | 4067 | N | LYS | A | 270 | 15.099 | 13.359 | 17.404 | 1.00 | 66.46 | N |
| ATOM | 4069 | CA | LYS | A | 270 | 15.659 | 13.544 | 18.749 | 1.00 | 67.95 | C |
| ATOM | 4071 | CB | LYS | A | 270 | 14.794 | 12.868 | 19.835 | 1.00 | 68.78 | C |
| ATOM | 4078 | C | LYS | A | 270 | 17.118 | 13.048 | 18.735 | 1.00 | 67.52 | C |
| ATOM | 4079 | O | LYS | A | 270 | 17.418 | 12.037 | 18.093 | 1.00 | 67.82 | O |
| ATOM | 4080 | N | CYS | A | 271 | 18.029 | 13.792 | 19.378 | 1.00 | 67.18 | N |
| ATOM | 4082 | CA | CYS | A | 271 | 19.404 | 13.329 | 19.533 | 1.00 | 67.14 | C |
| ATOM | 4084 | CB | CYS | A | 271 | 20.298 | 14.392 | 20.163 | 1.00 | 67.37 | C |
| ATOM | 4087 | SG | CYS | A | 271 | 20.613 | 15.867 | 19.148 | 1.00 | 64.80 | S |
| ATOM | 4088 | C | CYS | A | 271 | 19.474 | 12.059 | 20.385 | 1.00 | 69.08 | C |
| ATOM | 4089 | O | CYS | A | 271 | 18.591 | 11.760 | 21.157 | 1.00 | 69.41 | O |
| ATOM | 4090 | N | PRO | A | 272 | 20.500 | 11.265 | 20.163 | 1.00 | 70.38 | N |
| ATOM | 4091 | CA | PRO | A | 272 | 20.849 | 10.151 | 21.051 | 1.00 | 72.24 | C |
| ATOM | 4093 | CB | PRO | A | 272 | 22.117 | 9.577 | 20.427 | 1.00 | 72.83 | C |
| ATOM | 4096 | CG | PRO | A | 272 | 22.339 | 10.261 | 19.105 | 1.00 | 71.30 | C |
| ATOM | 4099 | CD | PRO | A | 272 | 21.334 | 11.334 | 18.950 | 1.00 | 69.52 | C |
| ATOM | 4102 | C | PRO | A | 272 | 21.148 | 10.500 | 22.518 | 1.00 | 74.35 | C |
| ATOM | 4103 | O | PRO | A | 272 | 21.335 | 11.680 | 22.899 | 1.00 | 72.50 | O |
| ATOM | 4104 | N | ALA | A | 273 | 21.207 | 9.460 | 23.360 | 1.00 | 76.92 | N |
| ATOM | 4106 | CA | ALA | A | 273 | 21.336 | 9.770 | 24.770 | 1.00 | 79.31 | C |
| ATOM | 4108 | CB | ALA | A | 273 | 21.267 | 8.471 | 25.704 | 1.00 | 81.70 | C |
| ATOM | 4112 | C | ALA | A | 273 | 22.660 | 10.549 | 24.920 | 1.00 | 80.08 | C |
| ATOM | 4113 | O | ALA | A | 273 | 23.657 | 10.214 | 24.269 | 1.00 | 81.25 | O |
| ATOM | 4114 | N | ALA | A | 274 | 22.643 | 11.606 | 25.731 | 1.00 | 80.37 | N |
| ATOM | 4116 | CA | ALA | A | 274 | 23.873 | 12.309 | 26.180 | 1.00 | 80.79 | C |
| ATOM | 4118 | CB | ALA | A | 274 | 24.953 | 11.284 | 26.709 | 1.00 | 82.92 | C |
| ATOM | 4122 | C | ALA | A | 274 | 24.467 | 13.290 | 25.124 | 1.00 | 79.12 | C |
| ATOM | 4123 | O | ALA | A | 274 | 25.525 | 13.931 | 25.359 | 1.00 | 80.24 | O |
| ATOM | 4124 | N | TYR | A | 275 | 23.770 | 13.426 | 23.984 | 1.00 | 76.37 | N |
| ATOM | 4126 | CA | TYR | A | 275 | 24.059 | 14.488 | 23.006 | 1.00 | 73.46 | C |
| ATOM | 4128 | CB | TYR | A | 275 | 23.851 | 13.945 | 21.589 | 1.00 | 72.66 | C |
| ATOM | 4131 | CG | TYR | A | 275 | 24.943 | 13.029 | 21.067 | 1.00 | 73.61 | C |
| ATOM | 4132 | CD1 | TYR | A | 275 | 24.814 | 11.666 | 21.160 | 1.00 | 75.81 | C |
| ATOM | 4134 | CE1 | TYR | A | 275 | 25.821 | 10.768 | 20.623 | 1.00 | 78.82 | C |
| ATOM | 4136 | CZ | TYR | A | 275 | 26.949 | 11.265 | 19.972 | 1.00 | 77.14 | C |
| ATOM | 4137 | OH | TYR | A | 275 | 27.922 | 10.378 | 19.496 | 1.00 | 74.15 | O |
| ATOM | 4139 | CE2 | TYR | A | 275 | 27.068 | 12.649 | 19.842 | 1.00 | 76.49 | C |
| ATOM | 4141 | CD2 | TYR | A | 275 | 26.066 | 13.529 | 20.396 | 1.00 | 74.32 | C |
| ATOM | 4143 | C | TYR | A | 275 | 23.253 | 15.819 | 23.224 | 1.00 | 70.60 | C |
| ATOM | 4144 | O | TYR | A | 275 | 22.179 | 15.869 | 23.817 | 1.00 | 69.08 | O |
| ATOM | 4145 | N | VAL | A | 276 | 23.819 | 16.887 | 22.710 | 1.00 | 68.49 | N |
| ATOM | 4147 | CA | VAL | A | 276 | 23.309 | 18.203 | 22.896 | 1.00 | 66.88 | C |
| ATOM | 4149 | CB | VAL | A | 276 | 24.361 | 19.009 | 23.644 | 1.00 | 67.40 | C |
| ATOM | 4151 | CG1 | VAL | A | 276 | 24.077 | 20.468 | 23.538 | 1.00 | 66.99 | C |
| ATOM | 4155 | CG2 | VAL | A | 276 | 24.424 | 18.581 | 25.105 | 1.00 | 69.12 | C |
| ATOM | 4159 | C | VAL | A | 276 | 23.119 | 18.799 | 21.516 | 1.00 | 65.33 | C |
| ATOM | 4160 | O | VAL | A | 276 | 23.945 | 18.554 | 20.648 | 1.00 | 64.97 | O |
| ATOM | 4161 | N | VAL | A | 277 | 22.077 | 19.608 | 21.316 | 1.00 | 64.18 | N |
| ATOM | 4163 | CA | VAL | A | 277 | 21.895 | 20.312 | 20.051 | 1.00 | 63.43 | C |
| ATOM | 4165 | CB | VAL | A | 277 | 20.423 | 20.537 | 19.793 | 1.00 | 63.64 | C |
| ATOM | 4167 | CG1 | VAL | A | 277 | 20.178 | 21.579 | 18.660 | 1.00 | 63.81 | C |
| ATOM | 4171 | CG2 | VAL | A | 277 | 19.719 | 19.232 | 19.503 | 1.00 | 63.12 | C |
| ATOM | 4175 | C | VAL | A | 277 | 22.591 | 21.672 | 20.042 | 1.00 | 63.46 | C |
| ATOM | 4176 | O | VAL | A | 277 | 22.630 | 22.344 | 20.998 | 1.00 | 64.27 | O |
| ATOM | 4177 | N | THR | A | 278 | 23.107 | 22.065 | 18.912 | 1.00 | 64.13 | N |
| ATOM | 4179 | CA | THR | A | 278 | 23.883 | 23.273 | 18.716 | 1.00 | 65.38 | C |
| ATOM | 4181 | CB | THR | A | 278 | 24.924 | 22.973 | 17.652 | 1.00 | 65.09 | C |
| ATOM | 4183 | OG1 | THR | A | 278 | 25.926 | 22.130 | 18.219 | 1.00 | 66.18 | O |
| ATOM | 4185 | CG2 | THR | A | 278 | 25.683 | 24.189 | 17.208 | 1.00 | 67.03 | C |
| ATOM | 4189 | C | THR | A | 278 | 22.963 | 24.272 | 18.124 | 1.00 | 66.33 | C |
| ATOM | 4190 | O | THR | A | 278 | 21.894 | 23.912 | 17.678 | 1.00 | 66.13 | O |
| ATOM | 4191 | N | ASP | A | 279 | 23.387 | 25.530 | 18.075 | 1.00 | 68.38 | N |
| ATOM | 4193 | CA | ASP | A | 279 | 22.622 | 26.565 | 17.371 | 1.00 | 69.35 | C |
| ATOM | 4195 | CB | ASP | A | 279 | 23.231 | 27.974 | 17.663 | 1.00 | 70.17 | C |
| ATOM | 4198 | CG | ASP | A | 279 | 22.622 | 28.619 | 18.927 | 1.00 | 74.12 | C |
| ATOM | 4199 | OD1 | ASP | A | 279 | 21.509 | 28.147 | 19.255 | 1.00 | 80.61 | O |
| ATOM | 4200 | OD2 | ASP | A | 279 | 23.129 | 29.541 | 19.668 | 1.00 | 75.22 | O |
| ATOM | 4201 | C | ASP | A | 279 | 22.387 | 26.333 | 15.837 | 1.00 | 68.57 | C |
| ATOM | 4202 | O | ASP | A | 279 | 21.493 | 26.966 | 15.271 | 1.00 | 69.77 | O |
| ATOM | 4203 | N | HIS | A | 280 | 23.158 | 25.484 | 15.155 | 1.00 | 67.36 | N |
| ATOM | 4205 | CA | HIS | A | 280 | 22.837 | 25.161 | 13.757 | 1.00 | 66.40 | C |
| ATOM | 4207 | CB | HIS | A | 280 | 24.111 | 24.930 | 12.934 | 1.00 | 67.43 | C |
| ATOM | 4210 | CG | HIS | A | 280 | 25.044 | 26.101 | 12.966 | 1.00 | 75.84 | C |
| ATOM | 4211 | ND1 | HIS | A | 280 | 24.803 | 27.271 | 12.246 | 1.00 | 83.72 | N |
| ATOM | 4213 | CE1 | HIS | A | 280 | 25.755 | 28.167 | 12.529 | 1.00 | 86.54 | C |
| ATOM | 4215 | NE2 | HIS | A | 280 | 26.613 | 27.618 | 13.393 | 1.00 | 86.77 | N |
| ATOM | 4217 | CD2 | HIS | A | 280 | 26.187 | 26.325 | 13.689 | 1.00 | 82.80 | C |
| ATOM | 4219 | C | HIS | A | 280 | 21.928 | 23.935 | 13.660 | 1.00 | 63.82 | C |
| ATOM | 4220 | O | HIS | A | 280 | 21.670 | 23.449 | 12.583 | 1.00 | 62.82 | O |
| ATOM | 4221 | N | GLY | A | 281 | 21.454 | 23.433 | 14.786 | 1.00 | 62.07 | N |
| ATOM | 4223 | CA | GLY | A | 281 | 20.711 | 22.192 | 14.803 | 1.00 | 60.37 | C |
| ATOM | 4226 | C | GLY | A | 281 | 21.450 | 20.874 | 14.599 | 1.00 | 58.17 | C |
| ATOM | 4227 | O | GLY | A | 281 | 20.872 | 19.957 | 14.053 | 1.00 | 54.70 | O |
| ATOM | 4228 | N | SER | A | 282 | 22.674 | 20.769 | 15.099 | 1.00 | 58.36 | N |
| ATOM | 4230 | CA | SER | A | 282 | 23.451 | 19.518 | 14.988 | 1.00 | 59.80 | C |
| ATOM | 4232 | CB | SER | A | 282 | 24.823 | 19.766 | 14.377 | 1.00 | 59.16 | C |
| ATOM | 4235 | OG | SER | A | 282 | 25.214 | 21.051 | 14.795 | 1.00 | 62.95 | O |
| ATOM | 4237 | C | SER | A | 282 | 23.673 | 18.870 | 16.322 | 1.00 | 60.81 | C |
| ATOM | 4238 | O | SER | A | 282 | 23.983 | 19.548 | 17.275 | 1.00 | 61.77 | O |
| ATOM | 4239 | N | CYS | A | 283 | 23.563 | 17.547 | 16.351 | 1.00 | 61.19 | N |
| ATOM | 4241 | CA | CYS | A | 283 | 23.886 | 16.751 | 17.517 | 1.00 | 62.86 | C |
| ATOM | 4243 | CB | CYS | A | 283 | 23.288 | 15.378 | 17.378 | 1.00 | 62.70 | C |
| ATOM | 4246 | SG | CYS | A | 283 | 21.524 | 15.593 | 17.275 | 1.00 | 63.42 | S |
| ATOM | 4247 | C | CYS | A | 283 | 25.378 | 16.643 | 17.738 | 1.00 | 64.27 | C |
| ATOM | 4248 | O | CYS | A | 283 | 26.086 | 15.979 | 17.042 | 1.00 | 64.51 | O |
| ATOM | 4249 | N | VAL | A | 284 | 25.827 | 17.322 | 18.756 | 1.00 | 65.87 | N |
| ATOM | 4251 | CA | VAL | A | 284 | 27.194 | 17.333 | 19.161 | 1.00 | 67.25 | C |
| ATOM | 4253 | CB | VAL | A | 284 | 27.645 | 18.783 | 19.075 | 1.00 | 66.98 | C |
| ATOM | 4255 | CG1 | VAL | A | 284 | 28.287 | 19.308 | 20.427 | 1.00 | 69.20 | C |
| ATOM | 4259 | CG2 | VAL | A | 284 | 28.515 | 18.989 | 17.847 | 1.00 | 65.40 | C |
| ATOM | 4263 | C | VAL | A | 284 | 27.288 | 16.742 | 20.591 | 1.00 | 69.52 | C |
| ATOM | 4264 | O | VAL | A | 284 | 26.303 | 16.509 | 21.280 | 1.00 | 68.60 | O |
| ATOM | 4265 | N | ARG | A | 285 | 28.494 | 16.492 | 21.043 | 1.00 | 72.61 | N |
| ATOM | 4267 | CA | ARG | A | 285 | 28.679 | 15.718 | 22.271 | 1.00 | 75.24 | C |
| ATOM | 4269 | CB | ARG | A | 285 | 30.043 | 15.046 | 22.222 | 1.00 | 77.38 | C |
| ATOM | 4272 | CG | ARG | A | 285 | 30.241 | 14.309 | 20.879 | 1.00 | 77.58 | C |
| ATOM | 4275 | CD | ARG | A | 285 | 31.506 | 13.520 | 20.741 | 1.00 | 79.64 | C |
| ATOM | 4278 | NE | ARG | A | 285 | 31.750 | 12.663 | 21.889 | 1.00 | 81.81 | N |
| ATOM | 4280 | CZ | ARG | A | 285 | 32.836 | 11.910 | 21.998 | 1.00 | 88.43 | C |
| ATOM | 4281 | NH1 | ARG | A | 285 | 33.742 | 11.916 | 21.013 | 1.00 | 89.46 | N |
| ATOM | 4284 | NH2 | ARG | A | 285 | 33.035 | 11.135 | 23.078 | 1.00 | 92.89 | N |
| ATOM | 4287 | C | ARG | A | 285 | 28.524 | 16.617 | 23.501 | 1.00 | 75.88 | C |
| ATOM | 4288 | O | ARG | A | 285 | 27.908 | 16.216 | 24.524 | 1.00 | 76.68 | O |
| ATOM | 4289 | N | ALA | A | 286 | 29.080 | 17.833 | 23.386 | 1.00 | 75.41 | N |
| ATOM | 4291 | CA | ALA | A | 286 | 29.095 | 18.805 | 24.477 | 1.00 | 74.95 | C |
| ATOM | 4293 | CB | ALA | A | 286 | 30.314 | 18.614 | 25.312 | 1.00 | 77.22 | C |
| ATOM | 4297 | C | ALA | A | 286 | 29.079 | 20.218 | 23.942 | 1.00 | 73.45 | C |
| ATOM | 4298 | O | ALA | A | 286 | 29.676 | 20.530 | 22.894 | 1.00 | 72.96 | O |
| ATOM | 4299 | N | CYS | A | 287 | 28.406 | 21.073 | 24.681 | 1.00 | 72.05 | N |
| ATOM | 4301 | CA | CYS | A | 287 | 28.399 | 22.468 | 24.351 | 1.00 | 71.75 | C |
| ATOM | 4303 | CB | CYS | A | 287 | 27.642 | 23.229 | 25.427 | 1.00 | 71.31 | C |
| ATOM | 4306 | SG | CYS | A | 287 | 25.875 | 22.940 | 25.188 | 1.00 | 73.43 | S |
| ATOM | 4307 | C | CYS | A | 287 | 29.768 | 23.066 | 24.284 | 1.00 | 72.75 | C |
| ATOM | 4308 | O | CYS | A | 287 | 30.617 | 22.701 | 25.063 | 1.00 | 73.85 | O |
| ATOM | 4309 | N | GLY | A | 288 | 29.938 | 24.051 | 23.398 | 1.00 | 72.69 | N |
| ATOM | 4311 | CA | GLY | A | 288 | 31.088 | 24.947 | 23.428 | 1.00 | 74.02 | C |
| ATOM | 4314 | C | GLY | A | 288 | 31.303 | 25.704 | 24.750 | 1.00 | 75.81 | C |
| ATOM | 4315 | O | GLY | A | 288 | 30.500 | 25.659 | 25.739 | 1.00 | 76.26 | O |
| ATOM | 4316 | N | ALA | A | 289 | 32.440 | 26.388 | 24.813 | 1.00 | 77.00 | N |
| ATOM | 4318 | CA | ALA | A | 289 | 32.570 | 27.523 | 25.728 | 1.00 | 76.07 | C |
| ATOM | 4320 | CB | ALA | A | 289 | 34.065 | 27.885 | 25.926 | 1.00 | 78.28 | C |
| ATOM | 4324 | C | ALA | A | 289 | 31.802 | 28.638 | 25.003 | 1.00 | 73.43 | C |
| ATOM | 4325 | O | ALA | A | 289 | 31.441 | 28.499 | 23.823 | 1.00 | 72.51 | O |
| ATOM | 4326 | N | GLU | A | 290 | 31.556 | 29.741 | 25.680 | 1.00 | 71.93 | N |
| ATOM | 4328 | CA | GLU | A | 290 | 30.674 | 30.784 | 25.138 | 1.00 | 69.93 | C |
| ATOM | 4330 | CB | GLU | A | 290 | 30.933 | 31.060 | 23.638 | 1.00 | 69.55 | C |
| ATOM | 4337 | C | GLU | A | 290 | 29.204 | 30.388 | 25.378 | 1.00 | 68.43 | C |
| ATOM | 4338 | O | GLU | A | 290 | 28.304 | 31.263 | 25.434 | 1.00 | 66.42 | O |
| ATOM | 4339 | N | SER | A | 291 | 28.965 | 29.076 | 25.564 | 1.00 | 68.05 | N |
| ATOM | 4341 | CA | SER | A | 291 | 27.586 | 28.561 | 25.670 | 1.00 | 66.93 | C |
| ATOM | 4343 | CB | SER | A | 291 | 27.133 | 27.982 | 24.310 | 1.00 | 66.63 | C |
| ATOM | 4346 | OG | SER | A | 291 | 27.904 | 26.832 | 23.974 | 1.00 | 70.01 | O |
| ATOM | 4348 | C | SER | A | 291 | 27.418 | 27.540 | 26.790 | 1.00 | 65.49 | C |
| ATOM | 4349 | O | SER | A | 291 | 28.386 | 27.171 | 27.433 | 1.00 | 65.82 | O |
| ATOM | 4350 | N | TYR | A | 292 | 26.187 | 27.099 | 27.003 | 1.00 | 63.29 | N |
| ATOM | 4352 | CA | TYR | A | 292 | 25.873 | 26.244 | 28.114 | 1.00 | 63.59 | C |
| ATOM | 4354 | CB | TYR | A | 292 | 25.605 | 27.105 | 29.361 | 1.00 | 63.27 | C |
| ATOM | 4357 | CG | TYR | A | 292 | 24.345 | 27.900 | 29.346 | 1.00 | 61.34 | C |
| ATOM | 4358 | CD1 | TYR | A | 292 | 23.232 | 27.426 | 29.953 | 1.00 | 61.28 | C |
| ATOM | 4360 | CE1 | TYR | A | 292 | 22.064 | 28.124 | 29.961 | 1.00 | 62.43 | C |
| ATOM | 4362 | CZ | TYR | A | 292 | 21.969 | 29.333 | 29.317 | 1.00 | 63.33 | C |
| ATOM | 4363 | OH | TYR | A | 292 | 20.753 | 30.010 | 29.369 | 1.00 | 61.75 | O |
| ATOM | 4365 | CE2 | TYR | A | 292 | 23.089 | 29.833 | 28.659 | 1.00 | 61.75 | C |
| ATOM | 4367 | CD2 | TYR | A | 292 | 24.269 | 29.109 | 28.695 | 1.00 | 61.15 | C |
| ATOM | 4369 | C | TYR | A | 292 | 24.680 | 25.364 | 27.824 | 1.00 | 63.55 | C |
| ATOM | 4370 | O | TYR | A | 292 | 23.902 | 25.665 | 26.944 | 1.00 | 61.46 | O |
| ATOM | 4371 | N | GLU | A | 293 | 24.514 | 24.283 | 28.594 | 1.00 | 65.22 | N |
| ATOM | 4373 | CA | GLU | A | 293 | 23.421 | 23.337 | 28.343 | 1.00 | 64.96 | C |
| ATOM | 4375 | CB | GLU | A | 293 | 23.822 | 21.946 | 28.802 | 1.00 | 66.42 | C |
| ATOM | 4378 | CG | GLU | A | 293 | 22.814 | 20.850 | 28.461 | 1.00 | 66.78 | C |
| ATOM | 4381 | CD | GLU | A | 293 | 23.357 | 19.453 | 28.686 | 1.00 | 68.32 | C |
| ATOM | 4382 | OE1 | GLU | A | 293 | 24.573 | 19.284 | 28.917 | 1.00 | 66.66 | O |
| ATOM | 4383 | OE2 | GLU | A | 293 | 22.541 | 18.514 | 28.585 | 1.00 | 72.49 | O |
| ATOM | 4384 | C | GLU | A | 293 | 22.180 | 23.740 | 29.062 | 1.00 | 64.18 | C |
| ATOM | 4385 | O | GLU | A | 293 | 22.258 | 24.258 | 30.131 | 1.00 | 64.53 | O |
| ATOM | 4386 | N | MET | A | 294 | 21.037 | 23.444 | 28.482 | 1.00 | 64.06 | N |
| ATOM | 4388 | CA | MET | A | 294 | 19.735 | 23.773 | 29.048 | 1.00 | 64.77 | C |
| ATOM | 4390 | CB | MET | A | 294 | 19.444 | 25.230 | 28.817 | 1.00 | 64.63 | C |
| ATOM | 4393 | CG | MET | A | 294 | 19.548 | 25.564 | 27.386 | 1.00 | 65.21 | C |
| ATOM | 4396 | SD | MET | A | 294 | 18.161 | 26.233 | 26.768 | 1.00 | 67.82 | S |
| ATOM | 4397 | CE | MET | A | 294 | 18.225 | 27.967 | 27.430 | 1.00 | 68.39 | C |
| ATOM | 4401 | C | MET | A | 294 | 18.693 | 22.959 | 28.335 | 1.00 | 65.12 | C |
| ATOM | 4402 | O | MET | A | 294 | 19.053 | 22.135 | 27.553 | 1.00 | 66.70 | O |
| ATOM | 4403 | N | GLU | A | 295 | 17.409 | 23.178 | 28.560 | 1.00 | 66.08 | N |
| ATOM | 4405 | CA | GLU | A | 295 | 16.403 | 22.270 | 28.025 | 1.00 | 66.85 | C |
| ATOM | 4407 | CB | GLU | A | 295 | 16.105 | 21.144 | 29.030 | 1.00 | 68.03 | C |
| ATOM | 4410 | CG | GLU | A | 295 | 15.320 | 19.987 | 28.420 | 1.00 | 70.00 | C |
| ATOM | 4413 | CD | GLU | A | 295 | 15.183 | 18.715 | 29.276 | 1.00 | 72.37 | C |
| ATOM | 4414 | OE1 | GLU | A | 295 | 15.186 | 18.767 | 30.490 | 1.00 | 69.76 | O |
| ATOM | 4415 | OE2 | GLU | A | 295 | 15.049 | 17.605 | 28.700 | 1.00 | 77.58 | O |
| ATOM | 4416 | C | GLU | A | 295 | 15.120 | 22.957 | 27.586 | 1.00 | 67.10 | C |
| ATOM | 4417 | O | GLU | A | 295 | 14.683 | 23.903 | 28.155 | 1.00 | 66.08 | O |
| ATOM | 4418 | N | GLU | A | 296 | 14.521 | 22.402 | 26.557 | 1.00 | 68.39 | N |
| ATOM | 4420 | CA | GLU | A | 296 | 13.398 | 22.997 | 25.876 | 1.00 | 69.98 | C |
| ATOM | 4422 | CB | GLU | A | 296 | 13.906 | 24.045 | 24.880 | 1.00 | 69.31 | C |
| ATOM | 4425 | CG | GLU | A | 296 | 12.939 | 25.175 | 24.543 | 1.00 | 71.32 | C |
| ATOM | 4428 | CD | GLU | A | 296 | 13.505 | 26.317 | 23.613 | 1.00 | 72.51 | C |
| ATOM | 4429 | OE1 | GLU | A | 296 | 14.592 | 26.270 | 22.931 | 1.00 | 67.50 | O |
| ATOM | 4430 | OE2 | GLU | A | 296 | 12.790 | 27.333 | 23.556 | 1.00 | 76.03 | O |
| ATOM | 4431 | C | GLU | A | 296 | 12.649 | 21.830 | 25.194 | 1.00 | 71.02 | C |
| ATOM | 4432 | O | GLU | A | 296 | 13.163 | 21.119 | 24.332 | 1.00 | 69.99 | O |
| ATOM | 4433 | N | ASP | A | 297 | 11.435 | 21.621 | 25.654 | 1.00 | 72.87 | N |
| ATOM | 4435 | CA | ASP | A | 297 | 10.564 | 20.526 | 25.225 | 1.00 | 74.19 | C |
| ATOM | 4437 | CB | ASP | A | 297 | 9.775 | 20.856 | 23.925 | 1.00 | 74.54 | C |
| ATOM | 4440 | CG | ASP | A | 297 | 8.253 | 20.479 | 24.078 | 1.00 | 78.91 | C |
| ATOM | 4441 | OD1 | ASP | A | 297 | 7.709 | 19.556 | 23.383 | 1.00 | 80.68 | O |
| ATOM | 4442 | OD2 | ASP | A | 297 | 7.528 | 21.033 | 24.964 | 1.00 | 83.36 | O |
| ATOM | 4443 | C | ASP | A | 297 | 11.184 | 19.131 | 25.212 | 1.00 | 73.07 | C |
| ATOM | 4444 | O | ASP | A | 297 | 10.949 | 18.325 | 24.330 | 1.00 | 72.94 | O |
| ATOM | 4445 | N | GLY | A | 298 | 11.922 | 18.833 | 26.251 | 1.00 | 72.78 | N |
| ATOM | 4447 | CA | GLY | A | 298 | 12.585 | 17.537 | 26.330 | 1.00 | 72.97 | C |
| ATOM | 4450 | C | GLY | A | 298 | 13.754 | 17.408 | 25.377 | 1.00 | 71.57 | C |
| ATOM | 4451 | O | GLY | A | 298 | 14.020 | 16.324 | 24.868 | 1.00 | 72.37 | O |
| ATOM | 4452 | N | VAL | A | 299 | 14.432 | 18.524 | 25.111 | 1.00 | 70.39 | N |
| ATOM | 4454 | CA | VAL | A | 299 | 15.601 | 18.557 | 24.223 | 1.00 | 68.75 | C |
| ATOM | 4456 | CB | VAL | A | 299 | 15.264 | 19.209 | 22.862 | 1.00 | 68.23 | C |
| ATOM | 4458 | CG1 | VAL | A | 299 | 16.357 | 18.967 | 21.894 | 1.00 | 67.60 | C |
| ATOM | 4462 | CG2 | VAL | A | 299 | 13.917 | 18.716 | 22.298 | 1.00 | 68.94 | C |
| ATOM | 4466 | C | VAL | A | 299 | 16.718 | 19.382 | 24.871 | 1.00 | 67.74 | C |
| ATOM | 4467 | O | VAL | A | 299 | 16.573 | 20.574 | 25.068 | 1.00 | 66.87 | O |
| ATOM | 4468 | N | ARG | A | 300 | 17.821 | 18.729 | 25.220 | 1.00 | 67.57 | N |
| ATOM | 4470 | CA | ARG | A | 300 | 19.013 | 19.425 | 25.667 | 1.00 | 66.47 | C |
| ATOM | 4472 | CB | ARG | A | 300 | 19.957 | 18.390 | 26.324 | 1.00 | 67.22 | C |
| ATOM | 4481 | C | ARG | A | 300 | 19.626 | 20.231 | 24.437 | 1.00 | 64.86 | C |
| ATOM | 4482 | O | ARG | A | 300 | 19.911 | 19.629 | 23.366 | 1.00 | 63.66 | O |
| ATOM | 4483 | N | LYS | A | 301 | 19.757 | 21.576 | 24.594 | 1.00 | 63.56 | N |
| ATOM | 4485 | CA | LYS | A | 301 | 20.400 | 22.506 | 23.623 | 1.00 | 62.09 | C |
| ATOM | 4487 | CB | LYS | A | 301 | 19.365 | 23.446 | 23.014 | 1.00 | 61.81 | C |
| ATOM | 4490 | CG | LYS | A | 301 | 18.004 | 22.874 | 22.945 | 1.00 | 62.27 | C |
| ATOM | 4493 | CD | LYS | A | 301 | 17.127 | 23.599 | 21.987 | 1.00 | 62.22 | C |
| ATOM | 4496 | CE | LYS | A | 301 | 15.767 | 22.883 | 21.889 | 1.00 | 64.18 | C |
| ATOM | 4499 | NZ | LYS | A | 301 | 14.711 | 23.900 | 21.583 | 1.00 | 68.36 | N |
| ATOM | 4503 | C | LYS | A | 301 | 21.474 | 23.415 | 24.208 | 1.00 | 61.40 | C |
| ATOM | 4504 | O | LYS | A | 301 | 21.486 | 23.655 | 25.364 | 1.00 | 61.73 | O |
| ATOM | 4505 | N | CYS | A | 302 | 22.354 | 23.943 | 23.380 | 1.00 | 60.84 | N |
| ATOM | 4507 | CA | CYS | A | 302 | 23.324 | 24.956 | 23.768 | 1.00 | 61.35 | C |
| ATOM | 4509 | CB | CYS | A | 302 | 24.550 | 24.881 | 22.905 | 1.00 | 60.99 | C |
| ATOM | 4512 | SG | CYS | A | 302 | 25.368 | 23.314 | 23.146 | 1.00 | 67.99 | S |
| ATOM | 4513 | C | CYS | A | 302 | 22.794 | 26.275 | 23.482 | 1.00 | 60.63 | C |
| ATOM | 4514 | O | CYS | A | 302 | 22.523 | 26.536 | 22.363 | 1.00 | 62.04 | O |
| ATOM | 4515 | N | ALA | A | 303 | 22.657 | 27.119 | 24.472 | 1.00 | 60.94 | N |
| ATOM | 4517 | CA | ALA | A | 303 | 22.390 | 28.522 | 24.292 | 1.00 | 60.90 | C |
| ATOM | 4519 | CB | ALA | A | 303 | 21.236 | 28.950 | 25.251 | 1.00 | 60.91 | C |
| ATOM | 4523 | C | ALA | A | 303 | 23.723 | 29.290 | 24.565 | 1.00 | 61.01 | C |
| ATOM | 4524 | O | ALA | A | 303 | 24.608 | 28.755 | 25.162 | 1.00 | 61.07 | O |
| ATOM | 4525 | N | LYS | A | 304 | 23.867 | 30.532 | 24.114 | 1.00 | 61.74 | N |
| ATOM | 4527 | CA | LYS | A | 304 | 25.101 | 31.306 | 24.314 | 1.00 | 62.76 | C |
| ATOM | 4529 | CB | LYS | A | 304 | 25.383 | 32.328 | 23.177 | 1.00 | 62.14 | C |
| ATOM | 4536 | C | LYS | A | 304 | 24.981 | 32.007 | 25.672 | 1.00 | 63.58 | C |
| ATOM | 4537 | O | LYS | A | 304 | 23.927 | 32.573 | 26.016 | 1.00 | 62.97 | O |
| ATOM | 4538 | N | CYS | A | 305 | 26.067 | 31.947 | 26.441 | 1.00 | 64.37 | N |
| ATOM | 4540 | CA | CYS | A | 305 | 26.054 | 32.434 | 27.809 | 1.00 | 64.66 | C |
| ATOM | 4542 | CB | CYS | A | 305 | 27.392 | 32.217 | 28.470 | 1.00 | 65.21 | C |
| ATOM | 4545 | SG | CYS | A | 305 | 27.739 | 30.498 | 28.766 | 1.00 | 63.94 | S |
| ATOM | 4546 | C | CYS | A | 305 | 25.781 | 33.878 | 27.864 | 1.00 | 64.83 | C |
| ATOM | 4547 | O | CYS | A | 305 | 26.343 | 34.630 | 27.095 | 1.00 | 64.68 | O |
| ATOM | 4548 | N | GLU | A | 306 | 24.936 | 34.253 | 28.814 | 1.00 | 66.05 | N |
| ATOM | 4550 | CA | GLU | A | 306 | 24.524 | 35.644 | 29.001 | 1.00 | 66.80 | C |
| ATOM | 4552 | CB | GLU | A | 306 | 23.563 | 35.751 | 30.202 | 1.00 | 66.67 | C |
| ATOM | 4559 | C | GLU | A | 306 | 25.812 | 36.489 | 29.193 | 1.00 | 67.43 | C |
| ATOM | 4560 | O | GLU | A | 306 | 26.353 | 37.052 | 28.177 | 1.00 | 68.24 | O |
| ATOM | 4561 | N | GLY | A | 307 | 26.321 | 36.534 | 30.441 | 1.00 | 65.77 | N |
| ATOM | 4563 | CA | GLY | A | 307 | 27.647 | 37.068 | 30.683 | 1.00 | 65.67 | C |
| ATOM | 4566 | C | GLY | A | 307 | 28.789 | 36.058 | 30.514 | 1.00 | 65.77 | C |
| ATOM | 4567 | O | GLY | A | 307 | 28.749 | 35.218 | 29.655 | 1.00 | 66.29 | O |
| ATOM | 4568 | N | PRO | A | 308 | 29.826 | 36.129 | 31.345 | 1.00 | 65.73 | N |
| ATOM | 4569 | CA | PRO | A | 308 | 30.736 | 34.998 | 31.452 | 1.00 | 65.66 | C |
| ATOM | 4571 | CB | PRO | A | 308 | 31.671 | 35.399 | 32.609 | 1.00 | 66.34 | C |
| ATOM | 4574 | CG | PRO | A | 308 | 31.364 | 36.818 | 32.999 | 1.00 | 65.33 | C |
| ATOM | 4577 | CD | PRO | A | 308 | 30.197 | 37.256 | 32.246 | 1.00 | 65.52 | C |
| ATOM | 4580 | C | PRO | A | 308 | 29.978 | 33.708 | 31.807 | 1.00 | 64.49 | C |
| ATOM | 4581 | O | PRO | A | 308 | 29.013 | 33.700 | 32.535 | 1.00 | 63.44 | O |
| ATOM | 4582 | N | CYS | A | 309 | 30.438 | 32.594 | 31.299 | 1.00 | 64.85 | N |
| ATOM | 4584 | CA | CYS | A | 309 | 29.805 | 31.354 | 31.627 | 1.00 | 64.54 | C |
| ATOM | 4586 | CB | CYS | A | 309 | 30.386 | 30.212 | 30.830 | 1.00 | 65.21 | C |
| ATOM | 4589 | SG | CYS | A | 309 | 29.788 | 30.212 | 29.155 | 1.00 | 66.57 | S |
| ATOM | 4590 | C | CYS | A | 309 | 29.997 | 31.025 | 33.032 | 1.00 | 64.83 | C |
| ATOM | 4591 | O | CYS | A | 309 | 30.976 | 31.344 | 33.634 | 1.00 | 65.61 | O |
| ATOM | 4592 | N | ARG | A | 310 | 29.058 | 30.285 | 33.547 | 1.00 | 60.31 | N |
| ATOM | 4594 | CA | ARG | A | 310 | 29.113 | 29.882 | 34.923 | 1.00 | 57.41 | C |
| ATOM | 4596 | CB | ARG | A | 310 | 27.799 | 29.192 | 35.275 | 1.00 | 58.70 | C |
| ATOM | 4599 | CG | ARG | A | 310 | 27.864 | 28.223 | 36.449 | 1.00 | 59.00 | C |
| ATOM | 4602 | CD | ARG | A | 310 | 26.495 | 27.931 | 37.089 | 1.00 | 61.96 | C |
| ATOM | 4605 | NE | ARG | A | 310 | 26.526 | 28.291 | 38.498 | 1.00 | 59.13 | N |
| ATOM | 4607 | CZ | ARG | A | 310 | 26.658 | 27.437 | 39.485 | 1.00 | 58.19 | C |
| ATOM | 4608 | NH1 | ARG | A | 310 | 26.733 | 26.108 | 39.288 | 1.00 | 62.56 | N |
| ATOM | 4611 | NH2 | ARG | A | 310 | 26.687 | 27.939 | 40.688 | 1.00 | 54.22 | N |
| ATOM | 4614 | C | ARG | A | 310 | 30.266 | 28.956 | 35.092 | 1.00 | 56.38 | C |
| ATOM | 4615 | O | ARG | A | 310 | 30.384 | 27.970 | 34.367 | 1.00 | 60.06 | O |
| ATOM | 4616 | N | LYS | A | 311 | 31.077 | 29.253 | 36.067 | 1.00 | 52.95 | N |
| ATOM | 4618 | CA | LYS | A | 311 | 32.237 | 28.466 | 36.405 | 1.00 | 53.16 | C |
| ATOM | 4620 | CB | LYS | A | 311 | 33.489 | 29.100 | 35.809 | 1.00 | 53.22 | C |
| ATOM | 4623 | CG | LYS | A | 311 | 34.691 | 28.185 | 35.636 | 1.00 | 54.72 | C |
| ATOM | 4626 | CD | LYS | A | 311 | 35.994 | 28.877 | 36.020 | 1.00 | 54.84 | C |
| ATOM | 4629 | CE | LYS | A | 311 | 37.190 | 28.137 | 35.562 | 1.00 | 55.94 | C |
| ATOM | 4632 | NZ | LYS | A | 311 | 37.492 | 28.559 | 34.153 | 1.00 | 61.52 | N |
| ATOM | 4636 | C | LYS | A | 311 | 32.370 | 28.399 | 37.919 | 1.00 | 51.04 | C |
| ATOM | 4637 | O | LYS | A | 311 | 32.461 | 29.412 | 38.626 | 1.00 | 48.96 | O |
| ATOM | 4638 | N | VAL | A | 312 | 32.389 | 27.190 | 38.441 | 1.00 | 52.10 | N |
| ATOM | 4640 | CA | VAL | A | 312 | 32.387 | 27.022 | 39.879 | 1.00 | 49.43 | C |
| ATOM | 4642 | CB | VAL | A | 312 | 31.419 | 25.856 | 40.214 | 1.00 | 51.43 | C |
| ATOM | 4644 | CG1 | VAL | A | 312 | 31.644 | 25.299 | 41.629 | 1.00 | 51.31 | C |
| ATOM | 4648 | CG2 | VAL | A | 312 | 29.941 | 26.303 | 40.019 | 1.00 | 51.87 | C |
| ATOM | 4652 | C | VAL | A | 312 | 33.810 | 26.792 | 40.321 | 1.00 | 47.46 | C |
| ATOM | 4653 | O | VAL | A | 312 | 34.534 | 26.134 | 39.633 | 1.00 | 48.81 | O |
| ATOM | 4654 | N | CYS | A | 313 | 34.224 | 27.342 | 41.452 | 1.00 | 45.64 | N |
| ATOM | 4656 | CA | CYS | A | 313 | 35.603 | 27.139 | 41.985 | 1.00 | 46.21 | C |
| ATOM | 4658 | CB | CYS | A | 313 | 36.533 | 28.305 | 41.639 | 1.00 | 45.10 | C |
| ATOM | 4661 | SG | CYS | A | 313 | 36.511 | 28.703 | 39.843 | 1.00 | 55.46 | S |
| ATOM | 4662 | C | CYS | A | 313 | 35.674 | 26.972 | 43.482 | 1.00 | 44.15 | C |
| ATOM | 4663 | O | CYS | A | 313 | 35.043 | 27.684 | 44.222 | 1.00 | 44.97 | O |
| ATOM | 4664 | N | ASN | A | 314 | 36.502 | 26.078 | 43.951 | 1.00 | 44.46 | N |
| ATOM | 4666 | CA | ASN | A | 314 | 36.817 | 25.995 | 45.346 | 1.00 | 42.36 | C |
| ATOM | 4668 | CB | ASN | A | 314 | 37.915 | 24.968 | 45.553 | 1.00 | 43.90 | C |
| ATOM | 4671 | CG | ASN | A | 314 | 37.515 | 23.563 | 45.052 | 1.00 | 46.81 | C |
| ATOM | 4672 | OD1 | ASN | A | 314 | 36.465 | 23.016 | 45.453 | 1.00 | 50.58 | O |
| ATOM | 4673 | ND2 | ASN | A | 314 | 38.309 | 23.026 | 44.099 | 1.00 | 49.45 | N |
| ATOM | 4676 | C | ASN | A | 314 | 37.278 | 27.306 | 45.972 | 1.00 | 40.41 | C |
| ATOM | 4677 | O | ASN | A | 314 | 37.974 | 28.140 | 45.385 | 1.00 | 40.47 | O |
| ATOM | 4678 | N | GLY | A | 315 | 36.860 | 27.494 | 47.209 | 1.00 | 39.42 | N |
| ATOM | 4680 | CA | GLY | A | 315 | 37.329 | 28.597 | 47.998 | 1.00 | 36.84 | C |
| ATOM | 4683 | C | GLY | A | 315 | 38.633 | 28.342 | 48.697 | 1.00 | 36.39 | C |
| ATOM | 4684 | O | GLY | A | 315 | 39.321 | 27.340 | 48.580 | 1.00 | 36.34 | O |
| ATOM | 4685 | N | ILE | A | 316 | 38.975 | 29.319 | 49.483 | 1.00 | 35.62 | N |
| ATOM | 4687 | CA | ILE | A | 316 | 40.197 | 29.200 | 50.208 | 1.00 | 36.69 | C |
| ATOM | 4689 | CB | ILE | A | 316 | 40.615 | 30.542 | 50.613 | 1.00 | 34.84 | C |
| ATOM | 4691 | CG1 | ILE | A | 316 | 41.327 | 31.048 | 49.383 | 1.00 | 37.92 | C |
| ATOM | 4694 | CD1 | ILE | A | 316 | 41.400 | 32.434 | 49.319 | 1.00 | 40.69 | C |
| ATOM | 4698 | CG2 | ILE | A | 316 | 41.647 | 30.485 | 51.734 | 1.00 | 34.92 | C |
| ATOM | 4702 | C | ILE | A | 316 | 40.093 | 28.246 | 51.344 | 1.00 | 37.13 | C |
| ATOM | 4703 | O | ILE | A | 316 | 39.246 | 28.398 | 52.197 | 1.00 | 36.17 | O |
| ATOM | 4704 | N | GLY | A | 317 | 40.969 | 27.271 | 51.351 | 1.00 | 38.50 | N |
| ATOM | 4706 | CA | GLY | A | 317 | 40.937 | 26.289 | 52.410 | 1.00 | 40.78 | C |
| ATOM | 4709 | C | GLY | A | 317 | 40.864 | 24.924 | 51.786 | 1.00 | 43.80 | C |
| ATOM | 4710 | O | GLY | A | 317 | 41.628 | 23.995 | 52.149 | 1.00 | 46.54 | O |
| ATOM | 4711 | N | ILE | A | 318 | 40.026 | 24.860 | 50.760 | 1.00 | 43.85 | N |
| ATOM | 4713 | CA | ILE | A | 318 | 39.762 | 23.645 | 49.983 | 1.00 | 46.17 | C |
| ATOM | 4715 | CB | ILE | A | 318 | 38.287 | 23.658 | 49.611 | 1.00 | 45.60 | C |
| ATOM | 4717 | CG1 | ILE | A | 318 | 37.441 | 23.639 | 50.888 | 1.00 | 43.98 | C |
| ATOM | 4720 | CD1 | ILE | A | 318 | 36.125 | 24.137 | 50.662 | 1.00 | 43.89 | C |
| ATOM | 4724 | CG2 | ILE | A | 318 | 38.020 | 22.487 | 48.661 | 1.00 | 47.89 | C |
| ATOM | 4728 | C | ILE | A | 318 | 40.520 | 23.509 | 48.639 | 1.00 | 47.63 | C |
| ATOM | 4729 | O | ILE | A | 318 | 40.833 | 24.543 | 48.012 | 1.00 | 46.75 | O |
| ATOM | 4730 | N | GLY | A | 319 | 40.735 | 22.254 | 48.176 | 1.00 | 49.13 | N |
| ATOM | 4732 | CA | GLY | A | 319 | 41.097 | 21.976 | 46.806 | 1.00 | 49.93 | C |
| ATOM | 4735 | C | GLY | A | 319 | 42.480 | 22.498 | 46.473 | 1.00 | 50.82 | C |
| ATOM | 4736 | O | GLY | A | 319 | 43.460 | 22.255 | 47.179 | 1.00 | 50.60 | O |
| ATOM | 4737 | N | GLU | A | 320 | 42.574 | 23.195 | 45.349 | 1.00 | 51.94 | N |
| ATOM | 4739 | CA | GLU | A | 320 | 43.811 | 23.859 | 44.916 | 1.00 | 52.89 | C |
| ATOM | 4741 | CB | GLU | A | 320 | 43.504 | 24.771 | 43.735 | 1.00 | 53.88 | C |
| ATOM | 4744 | CG | GLU | A | 320 | 42.802 | 24.123 | 42.555 | 1.00 | 59.54 | C |
| ATOM | 4747 | CD | GLU | A | 320 | 41.252 | 24.262 | 42.562 | 1.00 | 63.07 | C |
| ATOM | 4748 | OE1 | GLU | A | 320 | 40.603 | 24.715 | 43.485 | 1.00 | 57.30 | O |
| ATOM | 4749 | OE2 | GLU | A | 320 | 40.630 | 23.836 | 41.573 | 1.00 | 75.97 | O |
| ATOM | 4750 | C | GLU | A | 320 | 44.342 | 24.781 | 46.010 | 1.00 | 50.87 | C |
| ATOM | 4751 | O | GLU | A | 320 | 45.521 | 25.097 | 46.022 | 1.00 | 52.50 | O |
| ATOM | 4752 | N | TYR | A | 321 | 43.445 | 25.243 | 46.893 | 1.00 | 47.74 | N |
| ATOM | 4754 | CA | TYR | A | 321 | 43.796 | 25.996 | 48.048 | 1.00 | 45.39 | C |
| ATOM | 4756 | CB | TYR | A | 321 | 42.903 | 27.163 | 48.113 | 1.00 | 43.88 | C |
| ATOM | 4759 | CG | TYR | A | 321 | 42.549 | 27.735 | 46.790 | 1.00 | 47.78 | C |
| ATOM | 4760 | CD1 | TYR | A | 321 | 41.299 | 27.545 | 46.250 | 1.00 | 51.91 | C |
| ATOM | 4762 | CE1 | TYR | A | 321 | 40.963 | 28.042 | 45.044 | 1.00 | 54.97 | C |
| ATOM | 4764 | CZ | TYR | A | 321 | 41.861 | 28.800 | 44.366 | 1.00 | 58.00 | C |
| ATOM | 4765 | OH | TYR | A | 321 | 41.452 | 29.315 | 43.136 | 1.00 | 61.65 | O |
| ATOM | 4767 | CE2 | TYR | A | 321 | 43.129 | 29.019 | 44.881 | 1.00 | 55.75 | C |
| ATOM | 4769 | CD2 | TYR | A | 321 | 43.461 | 28.453 | 46.077 | 1.00 | 54.38 | C |
| ATOM | 4771 | C | TYR | A | 321 | 43.715 | 25.308 | 49.398 | 1.00 | 45.37 | C |
| ATOM | 4772 | O | TYR | A | 321 | 43.438 | 25.950 | 50.371 | 1.00 | 42.50 | O |
| ATOM | 4773 | N | LYS | A | 322 | 44.043 | 24.018 | 49.489 | 1.00 | 49.57 | N |
| ATOM | 4775 | CA | LYS | A | 322 | 44.267 | 23.354 | 50.814 | 1.00 | 50.70 | C |
| ATOM | 4777 | CB | LYS | A | 322 | 44.619 | 21.795 | 50.737 | 1.00 | 52.77 | C |
| ATOM | 4784 | C | LYS | A | 322 | 45.384 | 24.199 | 51.513 | 1.00 | 49.99 | C |
| ATOM | 4785 | O | LYS | A | 322 | 46.427 | 24.525 | 50.921 | 1.00 | 51.02 | O |
| ATOM | 4786 | N | ASP | A | 323 | 45.144 | 24.600 | 52.746 | 1.00 | 48.30 | N |
| ATOM | 4788 | CA | ASP | A | 323 | 46.253 | 25.036 | 53.583 | 1.00 | 47.80 | C |
| ATOM | 4790 | CB | ASP | A | 323 | 47.442 | 24.075 | 53.481 | 1.00 | 51.14 | C |
| ATOM | 4793 | CG | ASP | A | 323 | 47.180 | 22.729 | 54.109 | 1.00 | 53.64 | C |
| ATOM | 4794 | OD1 | ASP | A | 323 | 46.376 | 22.621 | 55.075 | 1.00 | 52.75 | O |
| ATOM | 4795 | OD2 | ASP | A | 323 | 47.796 | 21.713 | 53.701 | 1.00 | 58.75 | O |
| ATOM | 4796 | C | ASP | A | 323 | 46.770 | 26.367 | 53.240 | 1.00 | 45.65 | C |
| ATOM | 4797 | O | ASP | A | 323 | 47.821 | 26.714 | 53.723 | 1.00 | 47.47 | O |
| ATOM | 4798 | N | SER | A | 324 | 46.044 | 27.125 | 52.438 | 1.00 | 43.32 | N |
| ATOM | 4800 | CA | SER | A | 324 | 46.390 | 28.510 | 52.161 | 1.00 | 41.79 | C |
| ATOM | 4802 | CB | SER | A | 324 | 46.201 | 28.796 | 50.677 | 1.00 | 42.47 | C |
| ATOM | 4805 | OG | SER | A | 324 | 45.674 | 27.663 | 50.062 | 1.00 | 45.77 | O |
| ATOM | 4807 | C | SER | A | 324 | 45.537 | 29.476 | 52.905 | 1.00 | 37.75 | C |
| ATOM | 4808 | O | SER | A | 324 | 44.411 | 29.412 | 52.773 | 1.00 | 37.31 | O |
| ATOM | 4809 | N | LEU | A | 325 | 46.096 | 30.432 | 53.610 | 1.00 | 37.13 | N |
| ATOM | 4811 | CA | LEU | A | 325 | 45.334 | 31.298 | 54.493 | 1.00 | 35.04 | C |
| ATOM | 4813 | CB | LEU | A | 325 | 46.238 | 32.059 | 55.405 | 1.00 | 35.20 | C |
| ATOM | 4816 | CG | LEU | A | 325 | 47.124 | 31.230 | 56.304 | 1.00 | 38.21 | C |
| ATOM | 4818 | CD1 | LEU | A | 325 | 48.006 | 32.088 | 57.155 | 1.00 | 38.14 | C |
| ATOM | 4822 | CD2 | LEU | A | 325 | 46.216 | 30.335 | 57.130 | 1.00 | 38.92 | C |
| ATOM | 4826 | C | LEU | A | 325 | 44.533 | 32.325 | 53.776 | 1.00 | 33.99 | C |
| ATOM | 4827 | O | LEU | A | 325 | 43.445 | 32.619 | 54.178 | 1.00 | 32.49 | O |
| ATOM | 4828 | N | SER | A | 326 | 45.048 | 32.884 | 52.693 | 1.00 | 35.06 | N |
| ATOM | 4830 | CA | SER | A | 326 | 44.248 | 33.883 | 51.961 | 1.00 | 32.80 | C |
| ATOM | 4832 | CB | SER | A | 326 | 44.583 | 35.203 | 52.587 | 1.00 | 31.79 | C |
| ATOM | 4835 | OG | SER | A | 326 | 44.971 | 36.156 | 51.666 | 1.00 | 32.01 | O |
| ATOM | 4837 | C | SER | A | 326 | 44.404 | 33.929 | 50.432 | 1.00 | 33.06 | C |
| ATOM | 4838 | O | SER | A | 326 | 45.192 | 33.247 | 49.857 | 1.00 | 33.84 | O |
| ATOM | 4839 | N | ILE | A | 327 | 43.595 | 34.725 | 49.773 | 1.00 | 32.82 | N |
| ATOM | 4841 | CA | ILE | A | 327 | 43.767 | 34.943 | 48.360 | 1.00 | 34.47 | C |
| ATOM | 4843 | CB | ILE | A | 327 | 42.689 | 35.862 | 47.848 | 1.00 | 34.82 | C |
| ATOM | 4845 | CG1 | ILE | A | 327 | 41.498 | 35.102 | 47.344 | 1.00 | 36.75 | C |
| ATOM | 4848 | CD1 | ILE | A | 327 | 40.210 | 35.936 | 47.450 | 1.00 | 39.37 | C |
| ATOM | 4852 | CG2 | ILE | A | 327 | 43.072 | 36.537 | 46.591 | 1.00 | 38.67 | C |
| ATOM | 4856 | C | ILE | A | 327 | 45.050 | 35.640 | 48.320 | 1.00 | 34.57 | C |
| ATOM | 4857 | O | ILE | A | 327 | 45.288 | 36.446 | 49.182 | 1.00 | 34.42 | O |
| ATOM | 4858 | N | ASN | A | 328 | 45.886 | 35.347 | 47.332 | 1.00 | 36.41 | N |
| ATOM | 4860 | CA | ASN | A | 328 | 47.298 | 35.754 | 47.372 | 1.00 | 37.37 | C |
| ATOM | 4862 | CB | ASN | A | 328 | 48.002 | 35.038 | 48.522 | 1.00 | 37.48 | C |
| ATOM | 4865 | CG | ASN | A | 328 | 48.542 | 33.692 | 48.173 | 1.00 | 39.49 | C |
| ATOM | 4866 | OD1 | ASN | A | 328 | 49.033 | 33.448 | 47.064 | 1.00 | 46.88 | O |
| ATOM | 4867 | ND2 | ASN | A | 328 | 48.542 | 32.803 | 49.165 | 1.00 | 39.97 | N |
| ATOM | 4870 | C | ASN | A | 328 | 48.051 | 35.653 | 46.050 | 1.00 | 39.52 | C |
| ATOM | 4871 | O | ASN | A | 328 | 47.569 | 35.036 | 45.117 | 1.00 | 40.76 | O |
| ATOM | 4872 | N | ALA | A | 329 | 49.204 | 36.297 | 45.922 | 1.00 | 41.21 | N |
| ATOM | 4874 | CA | ALA | A | 329 | 49.894 | 36.340 | 44.585 | 1.00 | 44.38 | C |
| ATOM | 4876 | CB | ALA | A | 329 | 51.307 | 36.867 | 44.702 | 1.00 | 46.80 | C |
| ATOM | 4880 | C | ALA | A | 329 | 49.981 | 35.028 | 43.836 | 1.00 | 45.40 | C |
| ATOM | 4881 | O | ALA | A | 329 | 49.799 | 34.986 | 42.642 | 1.00 | 45.54 | O |
| ATOM | 4882 | N | THR | A | 330 | 50.308 | 33.986 | 44.577 | 1.00 | 45.70 | N |
| ATOM | 4884 | CA | THR | A | 330 | 50.416 | 32.677 | 44.025 | 1.00 | 48.70 | C |
| ATOM | 4886 | CB | THR | A | 330 | 50.997 | 31.669 | 45.089 | 1.00 | 49.87 | C |
| ATOM | 4888 | OG1 | THR | A | 330 | 52.331 | 32.048 | 45.543 | 1.00 | 51.51 | O |
| ATOM | 4890 | CG2 | THR | A | 330 | 51.204 | 30.298 | 44.458 | 1.00 | 50.43 | C |
| ATOM | 4894 | C | THR | A | 330 | 49.057 | 32.212 | 43.479 | 1.00 | 47.89 | C |
| ATOM | 4895 | O | THR | A | 330 | 48.914 | 31.950 | 42.295 | 1.00 | 51.27 | O |
| ATOM | 4896 | N | ASN | A | 331 | 48.053 | 32.168 | 44.321 | 1.00 | 44.82 | N |
| ATOM | 4898 | CA | ASN | A | 331 | 46.806 | 31.518 | 43.978 | 1.00 | 43.96 | C |
| ATOM | 4900 | CB | ASN | A | 331 | 46.163 | 30.942 | 45.241 | 1.00 | 41.18 | C |
| ATOM | 4903 | CG | ASN | A | 331 | 45.825 | 32.001 | 46.294 | 1.00 | 38.02 | C |
| ATOM | 4904 | OD1 | ASN | A | 331 | 45.247 | 33.040 | 46.021 | 1.00 | 35.91 | O |
| ATOM | 4905 | ND2 | ASN | A | 331 | 46.133 | 31.684 | 47.528 | 1.00 | 38.74 | N |
| ATOM | 4908 | C | ASN | A | 331 | 45.750 | 32.358 | 43.246 | 1.00 | 44.04 | C |
| ATOM | 4909 | O | ASN | A | 331 | 44.746 | 31.828 | 42.838 | 1.00 | 43.86 | O |
| ATOM | 4910 | N | ILE | A | 332 | 45.930 | 33.662 | 43.099 | 1.00 | 43.98 | N |
| ATOM | 4912 | CA | ILE | A | 332 | 44.823 | 34.466 | 42.632 | 1.00 | 42.84 | C |
| ATOM | 4914 | CB | ILE | A | 332 | 45.126 | 35.929 | 42.836 | 1.00 | 42.58 | C |
| ATOM | 4916 | CG1 | ILE | A | 332 | 44.037 | 36.797 | 42.283 | 1.00 | 44.23 | C |
| ATOM | 4919 | CD1 | ILE | A | 332 | 43.052 | 37.052 | 43.195 | 1.00 | 43.72 | C |
| ATOM | 4923 | CG2 | ILE | A | 332 | 46.249 | 36.384 | 42.012 | 1.00 | 47.11 | C |
| ATOM | 4927 | C | ILE | A | 332 | 44.578 | 34.128 | 41.178 | 1.00 | 45.50 | C |
| ATOM | 4928 | O | ILE | A | 332 | 43.489 | 34.240 | 40.691 | 1.00 | 45.59 | O |
| ATOM | 4929 | N | LYS | A | 333 | 45.597 | 33.700 | 40.442 | 1.00 | 49.20 | N |
| ATOM | 4931 | CA | LYS | A | 333 | 45.375 | 33.357 | 39.021 | 1.00 | 51.11 | C |
| ATOM | 4933 | CB | LYS | A | 333 | 46.690 | 33.013 | 38.339 | 1.00 | 54.36 | C |
| ATOM | 4936 | CG | LYS | A | 333 | 47.406 | 31.694 | 38.725 | 1.00 | 57.01 | C |
| ATOM | 4939 | CD | LYS | A | 333 | 48.821 | 31.590 | 37.959 | 1.00 | 62.54 | C |
| ATOM | 4942 | CE | LYS | A | 333 | 49.910 | 30.629 | 38.656 | 1.00 | 65.67 | C |
| ATOM | 4945 | NZ | LYS | A | 333 | 50.727 | 29.668 | 37.638 | 1.00 | 68.40 | N |
| ATOM | 4949 | C | LYS | A | 333 | 44.323 | 32.280 | 38.822 | 1.00 | 50.34 | C |
| ATOM | 4950 | O | LYS | A | 333 | 43.582 | 32.338 | 37.868 | 1.00 | 51.26 | O |
| ATOM | 4951 | N | HIS | A | 334 | 44.258 | 31.317 | 39.738 | 1.00 | 49.08 | N |
| ATOM | 4953 | CA | HIS | A | 334 | 43.312 | 30.206 | 39.685 | 1.00 | 49.27 | C |
| ATOM | 4955 | CB | HIS | A | 334 | 43.659 | 29.165 | 40.744 | 1.00 | 48.44 | C |
| ATOM | 4958 | CG | HIS | A | 334 | 44.980 | 28.524 | 40.539 | 1.00 | 53.18 | C |
| ATOM | 4959 | ND1 | HIS | A | 334 | 45.158 | 27.442 | 39.698 | 1.00 | 59.88 | N |
| ATOM | 4961 | CE1 | HIS | A | 334 | 46.441 | 27.119 | 39.674 | 1.00 | 62.27 | C |
| ATOM | 4963 | NE2 | HIS | A | 334 | 47.095 | 27.941 | 40.481 | 1.00 | 59.52 | N |
| ATOM | 4965 | CD2 | HIS | A | 334 | 46.199 | 28.819 | 41.045 | 1.00 | 54.75 | C |
| ATOM | 4967 | C | HIS | A | 334 | 41.853 | 30.601 | 39.909 | 1.00 | 47.75 | C |
| ATOM | 4968 | O | HIS | A | 334 | 40.986 | 29.750 | 39.847 | 1.00 | 48.67 | O |
| ATOM | 4969 | N | PHE | A | 335 | 41.579 | 31.874 | 40.217 | 1.00 | 46.14 | N |
| ATOM | 4971 | CA | PHE | A | 335 | 40.196 | 32.413 | 40.254 | 1.00 | 44.55 | C |
| ATOM | 4973 | CB | PHE | A | 335 | 40.053 | 33.427 | 41.378 | 1.00 | 41.24 | C |
| ATOM | 4976 | CG | PHE | A | 335 | 40.055 | 32.827 | 42.681 | 1.00 | 39.32 | C |
| ATOM | 4977 | CD1 | PHE | A | 335 | 41.223 | 32.551 | 43.307 | 1.00 | 39.57 | C |
| ATOM | 4979 | CE1 | PHE | A | 335 | 41.222 | 31.936 | 44.525 | 1.00 | 40.84 | C |
| ATOM | 4981 | CZ | PHE | A | 335 | 40.043 | 31.617 | 45.104 | 1.00 | 41.14 | C |
| ATOM | 4983 | CE2 | PHE | A | 335 | 38.863 | 31.842 | 44.455 | 1.00 | 40.09 | C |
| ATOM | 4985 | CD2 | PHE | A | 335 | 38.876 | 32.428 | 43.255 | 1.00 | 40.14 | C |
| ATOM | 4987 | C | PHE | A | 335 | 39.687 | 33.101 | 38.960 | 1.00 | 46.58 | C |
| ATOM | 4988 | O | PHE | A | 335 | 38.620 | 33.706 | 39.041 | 1.00 | 45.62 | O |
| ATOM | 4989 | N | LYS | A | 336 | 40.399 | 32.984 | 37.819 | 1.00 | 48.98 | N |
| ATOM | 4991 | CA | LYS | A | 336 | 40.046 | 33.678 | 36.590 | 1.00 | 51.49 | C |
| ATOM | 4993 | CB | LYS | A | 336 | 41.121 | 33.576 | 35.481 | 1.00 | 53.28 | C |
| ATOM | 5000 | C | LYS | A | 336 | 38.721 | 33.111 | 36.086 | 1.00 | 53.24 | C |
| ATOM | 5001 | O | LYS | A | 336 | 38.578 | 31.885 | 35.942 | 1.00 | 55.91 | O |
| ATOM | 5002 | N | ASN | A | 337 | 37.749 | 33.990 | 35.837 | 1.00 | 52.95 | N |
| ATOM | 5004 | CA | ASN | A | 337 | 36.461 | 33.566 | 35.321 | 1.00 | 54.28 | C |
| ATOM | 5006 | CB | ASN | A | 337 | 36.652 | 32.725 | 34.105 | 1.00 | 57.90 | C |
| ATOM | 5009 | CG | ASN | A | 337 | 37.513 | 33.373 | 33.127 | 1.00 | 61.13 | C |
| ATOM | 5010 | OD1 | ASN | A | 337 | 37.278 | 34.525 | 32.791 | 1.00 | 60.64 | O |
| ATOM | 5011 | ND2 | ASN | A | 337 | 38.553 | 32.662 | 32.664 | 1.00 | 67.20 | N |
| ATOM | 5014 | C | ASN | A | 337 | 35.581 | 32.785 | 36.259 | 1.00 | 52.45 | C |
| ATOM | 5015 | O | ASN | A | 337 | 34.540 | 32.290 | 35.832 | 1.00 | 52.89 | O |
| ATOM | 5016 | N | CYS | A | 338 | 35.970 | 32.691 | 37.527 | 1.00 | 49.80 | N |
| ATOM | 5018 | CA | CYS | A | 338 | 35.126 | 32.034 | 38.473 | 1.00 | 49.56 | C |
| ATOM | 5020 | CB | CYS | A | 338 | 35.803 | 31.803 | 39.799 | 1.00 | 47.52 | C |
| ATOM | 5023 | SG | CYS | A | 338 | 37.211 | 30.611 | 39.508 | 1.00 | 59.20 | S |
| ATOM | 5024 | C | CYS | A | 338 | 33.979 | 32.932 | 38.628 | 1.00 | 47.35 | C |
| ATOM | 5025 | O | CYS | A | 338 | 34.130 | 34.149 | 38.726 | 1.00 | 46.23 | O |
| ATOM | 5026 | N | THR | A | 339 | 32.834 | 32.301 | 38.627 | 1.00 | 46.93 | N |
| ATOM | 5028 | CA | THR | A | 339 | 31.599 | 32.968 | 38.809 | 1.00 | 47.23 | C |
| ATOM | 5030 | CB | THR | A | 339 | 30.770 | 32.593 | 37.622 | 1.00 | 50.51 | C |
| ATOM | 5032 | OG1 | THR | A | 339 | 30.642 | 33.782 | 36.857 | 1.00 | 53.34 | O |
| ATOM | 5034 | CG2 | THR | A | 339 | 29.337 | 32.064 | 37.962 | 1.00 | 50.61 | C |
| ATOM | 5038 | C | THR | A | 339 | 30.930 | 32.648 | 40.135 | 1.00 | 45.18 | C |
| ATOM | 5039 | O | THR | A | 339 | 30.247 | 33.470 | 40.689 | 1.00 | 44.47 | O |
| ATOM | 5040 | N | SER | A | 340 | 31.129 | 31.439 | 40.632 | 1.00 | 45.15 | N |
| ATOM | 5042 | CA | SER | A | 340 | 30.623 | 31.025 | 41.933 | 1.00 | 43.31 | C |
| ATOM | 5044 | CB | SER | A | 340 | 29.379 | 30.132 | 41.733 | 1.00 | 45.83 | C |
| ATOM | 5047 | OG | SER | A | 340 | 29.249 | 29.081 | 42.698 | 1.00 | 44.04 | O |
| ATOM | 5049 | C | SER | A | 340 | 31.702 | 30.248 | 42.678 | 1.00 | 41.72 | C |
| ATOM | 5050 | O | SER | A | 340 | 32.352 | 29.358 | 42.130 | 1.00 | 43.56 | O |
| ATOM | 5051 | N | ILE | A | 341 | 31.840 | 30.554 | 43.938 | 1.00 | 38.97 | N |
| ATOM | 5053 | CA | ILE | A | 341 | 32.891 | 30.013 | 44.722 | 1.00 | 38.11 | C |
| ATOM | 5055 | CB | ILE | A | 341 | 33.577 | 31.124 | 45.410 | 1.00 | 36.26 | C |
| ATOM | 5057 | CG1 | ILE | A | 341 | 34.447 | 31.785 | 44.365 | 1.00 | 37.48 | C |
| ATOM | 5060 | CD1 | ILE | A | 341 | 34.986 | 32.997 | 44.783 | 1.00 | 39.65 | C |
| ATOM | 5064 | CG2 | ILE | A | 341 | 34.370 | 30.578 | 46.573 | 1.00 | 36.38 | C |
| ATOM | 5068 | C | ILE | A | 341 | 32.279 | 29.147 | 45.704 | 1.00 | 38.22 | C |
| ATOM | 5069 | O | ILE | A | 341 | 31.424 | 29.588 | 46.497 | 1.00 | 36.50 | O |
| ATOM | 5070 | N | SER | A | 342 | 32.702 | 27.897 | 45.679 | 1.00 | 40.02 | N |
| ATOM | 5072 | CA | SER | A | 342 | 32.138 | 26.946 | 46.612 | 1.00 | 41.56 | C |
| ATOM | 5074 | CB | SER | A | 342 | 31.753 | 25.639 | 45.909 | 1.00 | 44.24 | C |
| ATOM | 5077 | OG | SER | A | 342 | 32.513 | 24.550 | 46.357 | 1.00 | 49.61 | O |
| ATOM | 5079 | C | SER | A | 342 | 33.114 | 26.871 | 47.782 | 1.00 | 39.51 | C |
| ATOM | 5080 | O | SER | A | 342 | 34.143 | 26.269 | 47.719 | 1.00 | 40.40 | O |
| ATOM | 5081 | N | GLY | A | 343 | 32.762 | 27.578 | 48.840 | 1.00 | 38.27 | N |
| ATOM | 5083 | CA | GLY | A | 343 | 33.645 | 27.834 | 49.938 | 1.00 | 37.00 | C |
| ATOM | 5086 | C | GLY | A | 343 | 33.681 | 29.322 | 50.263 | 1.00 | 35.53 | C |
| ATOM | 5087 | O | GLY | A | 343 | 32.754 | 30.074 | 49.877 | 1.00 | 34.59 | O |
| ATOM | 5088 | N | ASP | A | 344 | 34.775 | 29.727 | 50.970 | 1.00 | 34.54 | N |
| ATOM | 5090 | CA | ASP | A | 344 | 34.920 | 31.070 | 51.544 | 1.00 | 32.00 | C |
| ATOM | 5092 | CB | ASP | A | 344 | 35.234 | 30.977 | 53.032 | 1.00 | 31.72 | C |
| ATOM | 5095 | CG | ASP | A | 344 | 34.574 | 29.830 | 53.703 | 1.00 | 34.32 | C |
| ATOM | 5096 | OD1 | ASP | A | 344 | 33.317 | 29.772 | 53.622 | 1.00 | 41.66 | O |
| ATOM | 5097 | OD2 | ASP | A | 344 | 35.178 | 28.928 | 54.346 | 1.00 | 35.55 | O |
| ATOM | 5098 | C | ASP | A | 344 | 36.025 | 31.826 | 50.797 | 1.00 | 30.48 | C |
| ATOM | 5099 | O | ASP | A | 344 | 36.853 | 31.209 | 50.204 | 1.00 | 31.17 | O |
| ATOM | 5100 | N | LEU | A | 345 | 35.995 | 33.146 | 50.824 | 1.00 | 28.65 | N |
| ATOM | 5102 | CA | LEU | A | 345 | 37.056 | 33.969 | 50.344 | 1.00 | 29.65 | C |
| ATOM | 5104 | CB | LEU | A | 345 | 36.479 | 34.951 | 49.381 | 1.00 | 31.28 | C |
| ATOM | 5107 | CG | LEU | A | 345 | 36.877 | 35.111 | 47.916 | 1.00 | 34.49 | C |
| ATOM | 5109 | CD1 | LEU | A | 345 | 37.260 | 33.825 | 47.393 | 1.00 | 39.14 | C |
| ATOM | 5113 | CD2 | LEU | A | 345 | 35.690 | 35.571 | 47.155 | 1.00 | 33.77 | C |
| ATOM | 5117 | C | LEU | A | 345 | 37.580 | 34.737 | 51.548 | 1.00 | 29.18 | C |
| ATOM | 5118 | O | LEU | A | 345 | 36.784 | 35.296 | 52.295 | 1.00 | 29.37 | O |
| ATOM | 5119 | N | HIS | A | 346 | 38.882 | 34.655 | 51.810 | 1.00 | 29.00 | N |
| ATOM | 5121 | CA | HIS | A | 346 | 39.552 | 35.409 | 52.838 | 1.00 | 28.13 | C |
| ATOM | 5123 | CB | HIS | A | 346 | 40.486 | 34.542 | 53.655 | 1.00 | 29.47 | C |
| ATOM | 5126 | CG | HIS | A | 346 | 39.875 | 33.338 | 54.278 | 1.00 | 30.10 | C |
| ATOM | 5127 | ND1 | HIS | A | 346 | 38.833 | 33.396 | 55.118 | 1.00 | 35.14 | N |
| ATOM | 5129 | CE1 | HIS | A | 346 | 38.538 | 32.196 | 55.572 | 1.00 | 35.47 | C |
| ATOM | 5131 | NE2 | HIS | A | 346 | 39.391 | 31.361 | 55.089 | 1.00 | 32.97 | N |
| ATOM | 5133 | CD2 | HIS | A | 346 | 40.268 | 32.060 | 54.311 | 1.00 | 36.36 | C |
| ATOM | 5135 | C | HIS | A | 346 | 40.552 | 36.345 | 52.173 | 1.00 | 29.15 | C |
| ATOM | 5136 | O | HIS | A | 346 | 41.448 | 35.896 | 51.405 | 1.00 | 29.88 | O |
| ATOM | 5137 | N | ILE | A | 347 | 40.509 | 37.629 | 52.497 | 1.00 | 28.56 | N |
| ATOM | 5139 | CA | ILE | A | 347 | 41.537 | 38.505 | 52.005 | 1.00 | 28.80 | C |
| ATOM | 5141 | CB | ILE | A | 347 | 40.914 | 39.548 | 51.175 | 1.00 | 28.20 | C |
| ATOM | 5143 | CG1 | ILE | A | 347 | 40.355 | 38.876 | 49.939 | 1.00 | 28.97 | C |
| ATOM | 5146 | CD1 | ILE | A | 347 | 39.286 | 39.729 | 49.225 | 1.00 | 31.16 | C |
| ATOM | 5150 | CG2 | ILE | A | 347 | 41.951 | 40.589 | 50.781 | 1.00 | 29.70 | C |
| ATOM | 5154 | C | ILE | A | 347 | 42.248 | 39.072 | 53.183 | 1.00 | 28.89 | C |
| ATOM | 5155 | O | ILE | A | 347 | 41.697 | 39.924 | 53.810 | 1.00 | 29.99 | O |
| ATOM | 5156 | N | LEU | A | 348 | 43.443 | 38.605 | 53.524 | 1.00 | 30.00 | N |
| ATOM | 5158 | CA | LEU | A | 348 | 44.077 | 39.025 | 54.787 | 1.00 | 30.57 | C |
| ATOM | 5160 | CB | LEU | A | 348 | 44.555 | 37.886 | 55.605 | 1.00 | 30.53 | C |
| ATOM | 5163 | CG | LEU | A | 348 | 43.596 | 36.715 | 55.576 | 1.00 | 31.50 | C |
| ATOM | 5165 | CD1 | LEU | A | 348 | 44.246 | 35.482 | 56.211 | 1.00 | 32.92 | C |
| ATOM | 5169 | CD2 | LEU | A | 348 | 42.330 | 37.083 | 56.215 | 1.00 | 31.99 | C |
| ATOM | 5173 | C | LEU | A | 348 | 45.246 | 39.881 | 54.475 | 1.00 | 32.43 | C |
| ATOM | 5174 | O | LEU | A | 348 | 45.607 | 39.970 | 53.337 | 1.00 | 34.49 | O |
| ATOM | 5175 | N | PRO | A | 349 | 45.778 | 40.592 | 55.455 | 1.00 | 32.87 | N |
| ATOM | 5176 | CA | PRO | A | 349 | 46.973 | 41.381 | 55.264 | 1.00 | 33.99 | C |
| ATOM | 5178 | CB | PRO | A | 349 | 47.271 | 41.886 | 56.659 | 1.00 | 34.22 | C |
| ATOM | 5181 | CG | PRO | A | 349 | 45.966 | 42.137 | 57.153 | 1.00 | 34.18 | C |
| ATOM | 5184 | CD | PRO | A | 349 | 45.163 | 40.881 | 56.755 | 1.00 | 32.82 | C |
| ATOM | 5187 | C | PRO | A | 349 | 48.124 | 40.614 | 54.726 | 1.00 | 35.54 | C |
| ATOM | 5188 | O | PRO | A | 349 | 48.893 | 41.228 | 53.933 | 1.00 | 37.23 | O |
| ATOM | 5189 | N | VAL | A | 350 | 48.295 | 39.356 | 55.124 | 1.00 | 35.44 | N |
| ATOM | 5191 | CA | VAL | A | 350 | 49.450 | 38.608 | 54.559 | 1.00 | 37.52 | C |
| ATOM | 5193 | CB | VAL | A | 350 | 49.454 | 37.117 | 54.856 | 1.00 | 37.63 | C |
| ATOM | 5195 | CG1 | VAL | A | 350 | 50.325 | 36.871 | 55.983 | 1.00 | 39.53 | C |
| ATOM | 5199 | CG2 | VAL | A | 350 | 48.030 | 36.560 | 55.008 | 1.00 | 35.14 | C |
| ATOM | 5203 | C | VAL | A | 350 | 49.551 | 38.672 | 53.016 | 1.00 | 37.28 | C |
| ATOM | 5204 | O | VAL | A | 350 | 50.644 | 38.668 | 52.452 | 1.00 | 37.00 | O |
| ATOM | 5205 | N | ALA | A | 351 | 48.386 | 38.692 | 52.387 | 1.00 | 34.61 | N |
| ATOM | 5207 | CA | ALA | A | 351 | 48.304 | 38.858 | 50.982 | 1.00 | 35.88 | C |
| ATOM | 5209 | CB | ALA | A | 351 | 46.880 | 39.055 | 50.584 | 1.00 | 34.44 | C |
| ATOM | 5213 | C | ALA | A | 351 | 49.136 | 40.024 | 50.514 | 1.00 | 37.47 | C |
| ATOM | 5214 | O | ALA | A | 351 | 50.006 | 39.864 | 49.710 | 1.00 | 38.97 | O |
| ATOM | 5215 | N | PHE | A | 352 | 48.843 | 41.210 | 51.010 | 1.00 | 37.79 | N |
| ATOM | 5217 | CA | PHE | A | 352 | 49.495 | 42.407 | 50.491 | 1.00 | 39.28 | C |
| ATOM | 5219 | CB | PHE | A | 352 | 48.674 | 43.607 | 50.839 | 1.00 | 37.35 | C |
| ATOM | 5222 | CG | PHE | A | 352 | 47.296 | 43.459 | 50.390 | 1.00 | 35.68 | C |
| ATOM | 5223 | CD1 | PHE | A | 352 | 46.310 | 43.146 | 51.249 | 1.00 | 35.62 | C |
| ATOM | 5225 | CE1 | PHE | A | 352 | 45.040 | 42.971 | 50.821 | 1.00 | 35.56 | C |
| ATOM | 5227 | CZ | PHE | A | 352 | 44.760 | 43.037 | 49.504 | 1.00 | 37.20 | C |
| ATOM | 5229 | CE2 | PHE | A | 352 | 45.774 | 43.277 | 48.616 | 1.00 | 38.11 | C |
| ATOM | 5231 | CD2 | PHE | A | 352 | 47.016 | 43.468 | 49.070 | 1.00 | 38.39 | C |
| ATOM | 5233 | C | PHE | A | 352 | 50.912 | 42.564 | 50.942 | 1.00 | 42.10 | C |
| ATOM | 5234 | O | PHE | A | 352 | 51.661 | 43.264 | 50.308 | 1.00 | 45.13 | O |
| ATOM | 5235 | N | ARG | A | 353 | 51.318 | 41.916 | 52.018 | 1.00 | 42.10 | N |
| ATOM | 5237 | CA | ARG | A | 353 | 52.706 | 42.048 | 52.408 | 1.00 | 44.35 | C |
| ATOM | 5239 | CB | ARG | A | 353 | 52.810 | 42.476 | 53.863 | 1.00 | 44.18 | C |
| ATOM | 5242 | CG | ARG | A | 353 | 52.838 | 41.475 | 54.889 | 1.00 | 45.56 | C |
| ATOM | 5245 | CD | ARG | A | 353 | 52.340 | 41.993 | 56.328 | 1.00 | 46.05 | C |
| ATOM | 5248 | NE | ARG | A | 353 | 51.908 | 40.792 | 57.076 | 1.00 | 48.23 | N |
| ATOM | 5250 | CZ | ARG | A | 353 | 50.904 | 40.702 | 57.958 | 1.00 | 49.69 | C |
| ATOM | 5251 | NH1 | ARG | A | 353 | 50.159 | 41.766 | 58.367 | 1.00 | 48.00 | N |
| ATOM | 5254 | NH2 | ARG | A | 353 | 50.644 | 39.490 | 58.462 | 1.00 | 51.85 | N |
| ATOM | 5257 | C | ARG | A | 353 | 53.606 | 40.883 | 52.034 | 1.00 | 45.87 | C |
| ATOM | 5258 | O | ARG | A | 353 | 54.793 | 40.995 | 52.113 | 1.00 | 49.79 | O |
| ATOM | 5259 | N | GLY | A | 354 | 53.077 | 39.819 | 51.471 | 1.00 | 44.58 | N |
| ATOM | 5261 | CA | GLY | A | 354 | 53.844 | 38.608 | 51.326 | 1.00 | 45.57 | C |
| ATOM | 5264 | C | GLY | A | 354 | 54.077 | 37.881 | 52.646 | 1.00 | 45.59 | C |
| ATOM | 5265 | O | GLY | A | 354 | 53.795 | 38.404 | 53.757 | 1.00 | 44.40 | O |
| ATOM | 5266 | N | ASP | A | 355 | 54.560 | 36.657 | 52.489 | 1.00 | 47.02 | N |
| ATOM | 5268 | CA | ASP | A | 355 | 54.875 | 35.747 | 53.559 | 1.00 | 49.26 | C |
| ATOM | 5270 | CB | ASP | A | 355 | 53.675 | 34.810 | 53.761 | 1.00 | 47.45 | C |
| ATOM | 5273 | CG | ASP | A | 355 | 53.819 | 33.857 | 54.999 | 1.00 | 52.55 | C |
| ATOM | 5274 | OD1 | ASP | A | 355 | 54.748 | 33.966 | 55.843 | 1.00 | 52.58 | O |
| ATOM | 5275 | OD2 | ASP | A | 355 | 52.964 | 32.945 | 55.218 | 1.00 | 57.12 | O |
| ATOM | 5276 | C | ASP | A | 355 | 56.184 | 34.963 | 53.184 | 1.00 | 52.90 | C |
| ATOM | 5277 | O | ASP | A | 355 | 56.230 | 34.283 | 52.193 | 1.00 | 53.38 | O |
| ATOM | 5278 | N | SER | A | 356 | 57.257 | 35.097 | 53.951 | 1.00 | 55.22 | N |
| ATOM | 5280 | CA | SER | A | 356 | 58.449 | 34.379 | 53.620 | 1.00 | 59.39 | C |
| ATOM | 5282 | CB | SER | A | 356 | 59.693 | 35.013 | 54.276 | 1.00 | 62.91 | C |
| ATOM | 5285 | OG | SER | A | 356 | 59.657 | 34.911 | 55.688 | 1.00 | 63.67 | O |
| ATOM | 5287 | C | SER | A | 356 | 58.280 | 32.920 | 54.042 | 1.00 | 59.92 | C |
| ATOM | 5288 | O | SER | A | 356 | 58.935 | 31.998 | 53.497 | 1.00 | 62.62 | O |
| ATOM | 5289 | N | PHE | A | 357 | 57.411 | 32.695 | 55.015 | 1.00 | 57.30 | N |
| ATOM | 5291 | CA | PHE | A | 357 | 57.303 | 31.355 | 55.591 | 1.00 | 58.59 | C |
| ATOM | 5293 | CB | PHE | A | 357 | 56.605 | 31.427 | 56.964 | 1.00 | 57.36 | C |
| ATOM | 5296 | CG | PHE | A | 357 | 57.294 | 32.421 | 57.919 | 1.00 | 60.90 | C |
| ATOM | 5297 | CD1 | PHE | A | 357 | 56.748 | 33.697 | 58.164 | 1.00 | 59.90 | C |
| ATOM | 5299 | CE1 | PHE | A | 357 | 57.381 | 34.597 | 59.001 | 1.00 | 62.61 | C |
| ATOM | 5301 | CZ | PHE | A | 357 | 58.620 | 34.277 | 59.612 | 1.00 | 66.42 | C |
| ATOM | 5303 | CE2 | PHE | A | 357 | 59.197 | 33.034 | 59.358 | 1.00 | 70.78 | C |
| ATOM | 5305 | CD2 | PHE | A | 357 | 58.530 | 32.102 | 58.516 | 1.00 | 68.20 | C |
| ATOM | 5307 | C | PHE | A | 357 | 56.638 | 30.400 | 54.612 | 1.00 | 57.26 | C |
| ATOM | 5308 | O | PHE | A | 357 | 56.959 | 29.213 | 54.566 | 1.00 | 59.38 | O |
| ATOM | 5309 | N | THR | A | 358 | 55.781 | 30.990 | 53.792 | 1.00 | 53.74 | N |
| ATOM | 5311 | CA | THR | A | 358 | 54.993 | 30.317 | 52.788 | 1.00 | 52.07 | C |
| ATOM | 5313 | CB | THR | A | 358 | 53.638 | 30.968 | 52.871 | 1.00 | 48.69 | C |
| ATOM | 5315 | OG1 | THR | A | 358 | 52.917 | 30.251 | 53.847 | 1.00 | 49.41 | O |
| ATOM | 5317 | CG2 | THR | A | 358 | 52.782 | 30.701 | 51.766 | 1.00 | 49.09 | C |
| ATOM | 5321 | C | THR | A | 358 | 55.512 | 30.469 | 51.384 | 1.00 | 52.42 | C |
| ATOM | 5322 | O | THR | A | 358 | 54.936 | 29.873 | 50.469 | 1.00 | 50.78 | O |
| ATOM | 5323 | N | HIS | A | 359 | 56.595 | 31.258 | 51.252 | 1.00 | 53.66 | N |
| ATOM | 5325 | CA | HIS | A | 359 | 57.166 | 31.739 | 49.989 | 1.00 | 54.99 | C |
| ATOM | 5327 | CB | HIS | A | 359 | 57.756 | 30.604 | 49.243 | 1.00 | 57.50 | C |
| ATOM | 5330 | CG | HIS | A | 359 | 58.370 | 29.588 | 50.107 | 1.00 | 58.68 | C |
| ATOM | 5331 | ND1 | HIS | A | 359 | 59.709 | 29.584 | 50.363 | 1.00 | 63.58 | N |
| ATOM | 5333 | CE1 | HIS | A | 359 | 60.000 | 28.539 | 51.119 | 1.00 | 64.70 | C |
| ATOM | 5335 | NE2 | HIS | A | 359 | 58.883 | 27.896 | 51.392 | 1.00 | 61.34 | N |
| ATOM | 5337 | CD2 | HIS | A | 359 | 57.855 | 28.510 | 50.738 | 1.00 | 58.13 | C |
| ATOM | 5339 | C | HIS | A | 359 | 56.194 | 32.451 | 49.042 | 1.00 | 52.38 | C |
| ATOM | 5340 | O | HIS | A | 359 | 56.192 | 32.210 | 47.892 | 1.00 | 53.48 | O |
| ATOM | 5341 | N | THR | A | 360 | 55.402 | 33.359 | 49.534 | 1.00 | 50.00 | N |
| ATOM | 5343 | CA | THR | A | 360 | 54.434 | 34.040 | 48.722 | 1.00 | 48.70 | C |
| ATOM | 5345 | CB | THR | A | 360 | 53.195 | 33.835 | 49.430 | 1.00 | 45.16 | C |
| ATOM | 5347 | OG1 | THR | A | 360 | 52.938 | 32.444 | 49.359 | 1.00 | 46.70 | O |
| ATOM | 5349 | CG2 | THR | A | 360 | 52.036 | 34.453 | 48.731 | 1.00 | 44.91 | C |
| ATOM | 5353 | C | THR | A | 360 | 54.786 | 35.512 | 48.561 | 1.00 | 48.92 | C |
| ATOM | 5354 | O | THR | A | 360 | 54.978 | 36.188 | 49.533 | 1.00 | 48.33 | O |
| ATOM | 5355 | N | PRO | A | 361 | 55.010 | 35.986 | 47.351 | 1.00 | 50.79 | N |
| ATOM | 5356 | CA | PRO | A | 361 | 55.426 | 37.375 | 47.190 | 1.00 | 51.83 | C |
| ATOM | 5358 | CB | PRO | A | 361 | 55.759 | 37.484 | 45.706 | 1.00 | 54.65 | C |
| ATOM | 5361 | CG | PRO | A | 361 | 55.333 | 36.260 | 45.089 | 1.00 | 54.61 | C |
| ATOM | 5364 | CD | PRO | A | 361 | 55.017 | 35.248 | 46.088 | 1.00 | 52.35 | C |
| ATOM | 5367 | C | PRO | A | 361 | 54.285 | 38.263 | 47.500 | 1.00 | 48.71 | C |
| ATOM | 5368 | O | PRO | A | 361 | 53.182 | 37.783 | 47.427 | 1.00 | 47.40 | O |
| ATOM | 5369 | N | PRO | A | 362 | 54.507 | 39.526 | 47.794 | 1.00 | 48.27 | N |
| ATOM | 5370 | CA | PRO | A | 362 | 53.421 | 40.468 | 47.950 | 1.00 | 46.05 | C |
| ATOM | 5372 | CB | PRO | A | 362 | 54.162 | 41.791 | 48.029 | 1.00 | 47.43 | C |
| ATOM | 5375 | CG | PRO | A | 362 | 55.417 | 41.523 | 47.789 | 1.00 | 49.64 | C |
| ATOM | 5378 | CD | PRO | A | 362 | 55.780 | 40.167 | 48.028 | 1.00 | 50.35 | C |
| ATOM | 5381 | C | PRO | A | 362 | 52.473 | 40.437 | 46.765 | 1.00 | 45.74 | C |
| ATOM | 5382 | O | PRO | A | 362 | 52.943 | 40.284 | 45.696 | 1.00 | 49.40 | O |
| ATOM | 5383 | N | LEU | A | 363 | 51.189 | 40.595 | 46.966 | 1.00 | 44.33 | N |
| ATOM | 5385 | CA | LEU | A | 363 | 50.174 | 40.533 | 45.920 | 1.00 | 44.66 | C |
| ATOM | 5387 | CB | LEU | A | 363 | 48.831 | 40.112 | 46.510 | 1.00 | 41.08 | C |
| ATOM | 5390 | CG | LEU | A | 363 | 47.517 | 40.078 | 45.722 | 1.00 | 40.32 | C |
| ATOM | 5392 | CD1 | LEU | A | 363 | 47.505 | 39.026 | 44.654 | 1.00 | 41.76 | C |
| ATOM | 5396 | CD2 | LEU | A | 363 | 46.293 | 39.759 | 46.643 | 1.00 | 37.90 | C |
| ATOM | 5400 | C | LEU | A | 363 | 49.957 | 41.904 | 45.321 | 1.00 | 46.91 | C |
| ATOM | 5401 | O | LEU | A | 363 | 49.602 | 42.858 | 46.011 | 1.00 | 46.28 | O |
| ATOM | 5402 | N | ASP | A | 364 | 50.142 | 41.990 | 44.006 | 1.00 | 50.07 | N |
| ATOM | 5404 | CA | ASP | A | 364 | 49.742 | 43.191 | 43.278 | 1.00 | 50.91 | C |
| ATOM | 5406 | CB | ASP | A | 364 | 50.197 | 43.079 | 41.819 | 1.00 | 54.23 | C |
| ATOM | 5409 | CG | ASP | A | 364 | 49.881 | 44.322 | 40.987 | 1.00 | 56.02 | C |
| ATOM | 5410 | OD1 | ASP | A | 364 | 49.213 | 45.282 | 41.502 | 1.00 | 50.42 | O |
| ATOM | 5411 | OD2 | ASP | A | 364 | 50.278 | 44.378 | 39.776 | 1.00 | 60.00 | O |
| ATOM | 5412 | C | ASP | A | 364 | 48.221 | 43.399 | 43.404 | 1.00 | 47.27 | C |
| ATOM | 5413 | O | ASP | A | 364 | 47.433 | 42.672 | 42.883 | 1.00 | 45.88 | O |
| ATOM | 5414 | N | PRO | A | 365 | 47.821 | 44.436 | 44.058 | 1.00 | 46.25 | N |
| ATOM | 5415 | CA | PRO | A | 365 | 46.404 | 44.751 | 44.166 | 1.00 | 45.25 | C |
| ATOM | 5417 | CB | PRO | A | 365 | 46.408 | 46.030 | 44.972 | 1.00 | 45.77 | C |
| ATOM | 5420 | CG | PRO | A | 365 | 47.706 | 45.971 | 45.683 | 1.00 | 46.71 | C |
| ATOM | 5423 | CD | PRO | A | 365 | 48.660 | 45.428 | 44.709 | 1.00 | 47.17 | C |
| ATOM | 5426 | C | PRO | A | 365 | 45.610 | 45.003 | 42.889 | 1.00 | 47.18 | C |
| ATOM | 5427 | O | PRO | A | 365 | 44.431 | 44.806 | 43.003 | 1.00 | 46.77 | O |
| ATOM | 5428 | N | GLN | A | 366 | 46.155 | 45.502 | 41.778 | 1.00 | 50.90 | N |
| ATOM | 5430 | CA | GLN | A | 366 | 45.415 | 45.498 | 40.507 | 1.00 | 52.74 | C |
| ATOM | 5432 | CB | GLN | A | 366 | 46.289 | 45.759 | 39.264 | 1.00 | 56.32 | C |
| ATOM | 5439 | C | GLN | A | 366 | 44.779 | 44.131 | 40.329 | 1.00 | 51.62 | C |
| ATOM | 5440 | O | GLN | A | 366 | 43.737 | 44.006 | 39.686 | 1.00 | 53.12 | O |
| ATOM | 5441 | N | GLU | A | 367 | 45.392 | 43.090 | 40.873 | 1.00 | 49.93 | N |
| ATOM | 5443 | CA | GLU | A | 367 | 44.957 | 41.743 | 40.530 | 1.00 | 49.07 | C |
| ATOM | 5445 | CB | GLU | A | 367 | 46.016 | 40.748 | 40.955 | 1.00 | 49.47 | C |
| ATOM | 5448 | CG | GLU | A | 367 | 47.254 | 40.827 | 40.170 | 1.00 | 55.32 | C |
| ATOM | 5451 | CD | GLU | A | 367 | 47.521 | 39.537 | 39.415 | 1.00 | 65.30 | C |
| ATOM | 5452 | OE1 | GLU | A | 367 | 47.045 | 39.472 | 38.236 | 1.00 | 71.91 | O |
| ATOM | 5453 | OE2 | GLU | A | 367 | 48.219 | 38.601 | 39.984 | 1.00 | 69.48 | O |
| ATOM | 5454 | C | GLU | A | 367 | 43.609 | 41.341 | 41.121 | 1.00 | 44.37 | C |
| ATOM | 5455 | O | GLU | A | 367 | 43.026 | 40.320 | 40.783 | 1.00 | 43.44 | O |
| ATOM | 5456 | N | LEU | A | 368 | 43.146 | 42.122 | 42.045 | 1.00 | 41.59 | N |
| ATOM | 5458 | CA | LEU | A | 368 | 41.862 | 41.872 | 42.622 | 1.00 | 39.34 | C |
| ATOM | 5460 | CB | LEU | A | 368 | 41.649 | 42.755 | 43.821 | 1.00 | 37.40 | C |
| ATOM | 5463 | CG | LEU | A | 368 | 42.571 | 42.306 | 44.922 | 1.00 | 37.05 | C |
| ATOM | 5465 | CD1 | LEU | A | 368 | 42.591 | 43.311 | 46.006 | 1.00 | 37.85 | C |
| ATOM | 5469 | CD2 | LEU | A | 368 | 42.136 | 40.982 | 45.456 | 1.00 | 37.21 | C |
| ATOM | 5473 | C | LEU | A | 368 | 40.775 | 42.088 | 41.612 | 1.00 | 40.58 | C |
| ATOM | 5474 | O | LEU | A | 368 | 39.749 | 41.513 | 41.759 | 1.00 | 39.88 | O |
| ATOM | 5475 | N | ASP | A | 369 | 41.013 | 42.861 | 40.558 | 1.00 | 44.30 | N |
| ATOM | 5477 | CA | ASP | A | 369 | 40.101 | 42.958 | 39.417 | 1.00 | 46.16 | C |
| ATOM | 5479 | CB | ASP | A | 369 | 40.752 | 43.678 | 38.251 | 1.00 | 50.23 | C |
| ATOM | 5482 | CG | ASP | A | 369 | 41.010 | 45.227 | 38.506 | 1.00 | 55.84 | C |
| ATOM | 5483 | OD1 | ASP | A | 369 | 40.507 | 45.849 | 39.505 | 1.00 | 57.91 | O |
| ATOM | 5484 | OD2 | ASP | A | 369 | 41.782 | 45.893 | 37.753 | 1.00 | 60.79 | O |
| ATOM | 5485 | C | ASP | A | 369 | 39.621 | 41.612 | 38.939 | 1.00 | 45.71 | C |
| ATOM | 5486 | O | ASP | A | 369 | 38.531 | 41.537 | 38.475 | 1.00 | 46.84 | O |
| ATOM | 5487 | N | ILE | A | 370 | 40.412 | 40.553 | 39.067 | 1.00 | 45.58 | N |
| ATOM | 5489 | CA | ILE | A | 370 | 40.011 | 39.148 | 38.769 | 1.00 | 46.13 | C |
| ATOM | 5491 | CB | ILE | A | 370 | 41.135 | 38.210 | 39.219 | 1.00 | 46.46 | C |
| ATOM | 5493 | CG1 | ILE | A | 370 | 42.075 | 37.803 | 38.072 | 1.00 | 50.66 | C |
| ATOM | 5496 | CD1 | ILE | A | 370 | 43.582 | 37.835 | 38.532 | 1.00 | 51.51 | C |
| ATOM | 5500 | CG2 | ILE | A | 370 | 40.622 | 36.949 | 39.735 | 1.00 | 47.70 | C |
| ATOM | 5504 | C | ILE | A | 370 | 38.727 | 38.626 | 39.419 | 1.00 | 43.97 | C |
| ATOM | 5505 | O | ILE | A | 370 | 37.935 | 37.986 | 38.824 | 1.00 | 45.31 | O |
| ATOM | 5506 | N | LEU | A | 371 | 38.532 | 38.885 | 40.675 | 1.00 | 41.68 | N |
| ATOM | 5508 | CA | LEU | A | 371 | 37.290 | 38.530 | 41.354 | 1.00 | 39.69 | C |
| ATOM | 5510 | CB | LEU | A | 371 | 37.476 | 38.789 | 42.863 | 1.00 | 37.02 | C |
| ATOM | 5513 | CG | LEU | A | 371 | 38.785 | 38.136 | 43.352 | 1.00 | 36.34 | C |
| ATOM | 5515 | CD1 | LEU | A | 371 | 39.215 | 38.574 | 44.651 | 1.00 | 34.34 | C |
| ATOM | 5519 | CD2 | LEU | A | 371 | 38.586 | 36.658 | 43.371 | 1.00 | 38.01 | C |
| ATOM | 5523 | C | LEU | A | 371 | 36.031 | 39.257 | 40.875 | 1.00 | 40.31 | C |
| ATOM | 5524 | O | LEU | A | 371 | 34.962 | 38.940 | 41.363 | 1.00 | 38.85 | O |
| ATOM | 5525 | N | LYS | A | 372 | 36.131 | 40.222 | 39.959 | 1.00 | 42.21 | N |
| ATOM | 5527 | CA | LYS | A | 372 | 34.932 | 40.898 | 39.422 | 1.00 | 43.84 | C |
| ATOM | 5529 | CB | LYS | A | 372 | 35.285 | 41.942 | 38.377 | 1.00 | 46.36 | C |
| ATOM | 5532 | CG | LYS | A | 372 | 35.513 | 43.305 | 38.954 | 1.00 | 47.88 | C |
| ATOM | 5535 | CD | LYS | A | 372 | 36.436 | 44.155 | 38.058 | 1.00 | 51.39 | C |
| ATOM | 5538 | CE | LYS | A | 372 | 36.918 | 45.449 | 38.779 | 1.00 | 53.07 | C |
| ATOM | 5541 | NZ | LYS | A | 372 | 38.090 | 46.150 | 38.045 | 1.00 | 59.80 | N |
| ATOM | 5545 | C | LYS | A | 372 | 33.950 | 39.980 | 38.766 | 1.00 | 44.83 | C |
| ATOM | 5546 | O | LYS | A | 372 | 32.860 | 40.345 | 38.495 | 1.00 | 45.62 | O |
| ATOM | 5547 | N | THR | A | 373 | 34.355 | 38.775 | 38.458 | 1.00 | 46.35 | N |
| ATOM | 5549 | CA | THR | A | 373 | 33.487 | 37.815 | 37.799 | 1.00 | 47.94 | C |
| ATOM | 5551 | CB | THR | A | 373 | 34.382 | 36.802 | 37.192 | 1.00 | 48.66 | C |
| ATOM | 5553 | OG1 | THR | A | 373 | 35.052 | 37.419 | 36.130 | 1.00 | 51.06 | O |
| ATOM | 5555 | CG2 | THR | A | 373 | 33.564 | 35.865 | 36.391 | 1.00 | 55.55 | C |
| ATOM | 5559 | C | THR | A | 373 | 32.508 | 37.084 | 38.700 | 1.00 | 45.96 | C |
| ATOM | 5560 | O | THR | A | 373 | 31.617 | 36.372 | 38.216 | 1.00 | 48.41 | O |
| ATOM | 5561 | N | VAL | A | 374 | 32.706 | 37.253 | 39.998 | 1.00 | 42.63 | N |
| ATOM | 5563 | CA | VAL | A | 374 | 32.140 | 36.424 | 41.020 | 1.00 | 40.86 | C |
| ATOM | 5565 | CB | VAL | A | 374 | 33.088 | 36.339 | 42.283 | 1.00 | 38.33 | C |
| ATOM | 5567 | CG1 | VAL | A | 374 | 32.534 | 35.381 | 43.293 | 1.00 | 37.92 | C |
| ATOM | 5571 | CG2 | VAL | A | 374 | 34.458 | 35.804 | 41.921 | 1.00 | 38.98 | C |
| ATOM | 5575 | C | VAL | A | 374 | 30.751 | 36.893 | 41.416 | 1.00 | 40.06 | C |
| ATOM | 5576 | O | VAL | A | 374 | 30.539 | 38.005 | 41.826 | 1.00 | 39.99 | O |
| ATOM | 5577 | N | LYS | A | 375 | 29.823 | 35.983 | 41.353 | 1.00 | 40.81 | N |
| ATOM | 5579 | CA | LYS | A | 375 | 28.452 | 36.273 | 41.649 | 1.00 | 42.35 | C |
| ATOM | 5581 | CB | LYS | A | 375 | 27.541 | 35.795 | 40.477 | 1.00 | 46.59 | C |
| ATOM | 5584 | CG | LYS | A | 375 | 27.777 | 36.549 | 39.090 | 1.00 | 50.24 | C |
| ATOM | 5587 | CD | LYS | A | 375 | 26.669 | 36.227 | 38.059 | 1.00 | 57.80 | C |
| ATOM | 5590 | CE | LYS | A | 375 | 27.128 | 36.178 | 36.545 | 1.00 | 62.46 | C |
| ATOM | 5593 | NZ | LYS | A | 375 | 26.016 | 36.062 | 35.526 | 1.00 | 61.68 | N |
| ATOM | 5597 | C | LYS | A | 375 | 28.008 | 35.654 | 42.952 | 1.00 | 40.06 | C |
| ATOM | 5598 | O | LYS | A | 375 | 27.173 | 36.205 | 43.624 | 1.00 | 39.08 | O |
| ATOM | 5599 | N | GLU | A | 376 | 28.528 | 34.503 | 43.309 | 1.00 | 39.23 | N |
| ATOM | 5601 | CA | GLU | A | 376 | 28.081 | 33.914 | 44.552 | 1.00 | 39.53 | C |
| ATOM | 5603 | CB | GLU | A | 376 | 26.947 | 32.867 | 44.334 | 1.00 | 41.78 | C |
| ATOM | 5606 | CG | GLU | A | 376 | 27.373 | 31.519 | 43.819 | 1.00 | 44.47 | C |
| ATOM | 5609 | CD | GLU | A | 376 | 26.253 | 30.494 | 43.764 | 1.00 | 47.77 | C |
| ATOM | 5610 | OE1 | GLU | A | 376 | 25.436 | 30.495 | 44.646 | 1.00 | 51.29 | O |
| ATOM | 5611 | OE2 | GLU | A | 376 | 26.180 | 29.681 | 42.851 | 1.00 | 49.03 | O |
| ATOM | 5612 | C | GLU | A | 376 | 29.214 | 33.326 | 45.341 | 1.00 | 37.53 | C |
| ATOM | 5613 | O | GLU | A | 376 | 30.135 | 32.819 | 44.752 | 1.00 | 38.28 | O |
| ATOM | 5614 | N | LEU | A | 377 | 29.159 | 33.465 | 46.662 | 1.00 | 35.71 | N |
| ATOM | 5616 | CA | LEU | A | 377 | 29.980 | 32.695 | 47.555 | 1.00 | 35.03 | C |
| ATOM | 5618 | CB | LEU | A | 377 | 30.888 | 33.582 | 48.445 | 1.00 | 32.95 | C |
| ATOM | 5621 | CG | LEU | A | 377 | 31.383 | 34.875 | 47.722 | 1.00 | 32.32 | C |
| ATOM | 5623 | CD1 | LEU | A | 377 | 32.373 | 35.705 | 48.488 | 1.00 | 30.13 | C |
| ATOM | 5627 | CD2 | LEU | A | 377 | 32.034 | 34.537 | 46.491 | 1.00 | 36.27 | C |
| ATOM | 5631 | C | LEU | A | 377 | 29.001 | 31.854 | 48.361 | 1.00 | 36.88 | C |
| ATOM | 5632 | O | LEU | A | 377 | 28.064 | 32.351 | 48.983 | 1.00 | 37.14 | O |
| ATOM | 5633 | N | THR | A | 378 | 29.199 | 30.548 | 48.278 | 1.00 | 38.82 | N |
| ATOM | 5635 | CA | THR | A | 378 | 28.558 | 29.513 | 49.108 | 1.00 | 39.57 | C |
| ATOM | 5637 | CB | THR | A | 378 | 29.221 | 28.290 | 48.593 | 1.00 | 41.13 | C |
| ATOM | 5639 | OG1 | THR | A | 378 | 28.353 | 27.866 | 47.593 | 1.00 | 41.94 | O |
| ATOM | 5641 | CG2 | THR | A | 378 | 29.395 | 27.056 | 49.550 | 1.00 | 44.08 | C |
| ATOM | 5645 | C | THR | A | 378 | 28.753 | 29.721 | 50.604 | 1.00 | 37.78 | C |
| ATOM | 5646 | O | THR | A | 378 | 27.843 | 29.535 | 51.415 | 1.00 | 36.72 | O |
| ATOM | 5647 | N | GLY | A | 379 | 29.962 | 30.195 | 50.908 | 1.00 | 36.56 | N |
| ATOM | 5649 | CA | GLY | A | 379 | 30.487 | 30.357 | 52.264 | 1.00 | 35.72 | C |
| ATOM | 5652 | C | GLY | A | 379 | 30.468 | 31.774 | 52.761 | 1.00 | 33.26 | C |
| ATOM | 5653 | O | GLY | A | 379 | 29.453 | 32.421 | 52.744 | 1.00 | 34.48 | O |
| ATOM | 5654 | N | PHE | A | 380 | 31.604 | 32.269 | 53.181 | 1.00 | 31.43 | N |
| ATOM | 5656 | CA | PHE | A | 380 | 31.641 | 33.606 | 53.748 | 1.00 | 30.14 | C |
| ATOM | 5658 | CB | PHE | A | 380 | 31.949 | 33.563 | 55.237 | 1.00 | 29.78 | C |
| ATOM | 5661 | CG | PHE | A | 380 | 33.326 | 33.040 | 55.606 | 1.00 | 28.19 | C |
| ATOM | 5662 | CD1 | PHE | A | 380 | 34.468 | 33.856 | 55.512 | 1.00 | 26.22 | C |
| ATOM | 5664 | CE1 | PHE | A | 380 | 35.680 | 33.405 | 55.974 | 1.00 | 25.15 | C |
| ATOM | 5666 | CZ | PHE | A | 380 | 35.755 | 32.140 | 56.510 | 1.00 | 26.34 | C |
| ATOM | 5668 | CE2 | PHE | A | 380 | 34.631 | 31.325 | 56.573 | 1.00 | 27.52 | C |
| ATOM | 5670 | CD2 | PHE | A | 380 | 33.459 | 31.771 | 56.148 | 1.00 | 27.63 | C |
| ATOM | 5672 | C | PHE | A | 380 | 32.649 | 34.455 | 53.086 | 1.00 | 28.80 | C |
| ATOM | 5673 | O | PHE | A | 380 | 33.535 | 33.928 | 52.414 | 1.00 | 30.25 | O |
| ATOM | 5674 | N | LEU | A | 381 | 32.580 | 35.737 | 53.345 | 1.00 | 26.76 | N |
| ATOM | 5676 | CA | LEU | A | 381 | 33.462 | 36.629 | 52.687 | 1.00 | 27.56 | C |
| ATOM | 5678 | CB | LEU | A | 381 | 32.671 | 37.545 | 51.773 | 1.00 | 27.59 | C |
| ATOM | 5681 | CG | LEU | A | 381 | 33.418 | 38.759 | 51.282 | 1.00 | 28.54 | C |
| ATOM | 5683 | CD1 | LEU | A | 381 | 34.748 | 38.298 | 50.721 | 1.00 | 30.46 | C |
| ATOM | 5687 | CD2 | LEU | A | 381 | 32.616 | 39.439 | 50.209 | 1.00 | 32.21 | C |
| ATOM | 5691 | C | LEU | A | 381 | 34.189 | 37.411 | 53.743 | 1.00 | 27.21 | C |
| ATOM | 5692 | O | LEU | A | 381 | 33.594 | 38.246 | 54.431 | 1.00 | 28.39 | O |
| ATOM | 5693 | N | LEU | A | 382 | 35.471 | 37.202 | 53.868 | 1.00 | 26.93 | N |
| ATOM | 5695 | CA | LEU | A | 382 | 36.188 | 37.838 | 54.953 | 1.00 | 27.83 | C |
| ATOM | 5697 | CB | LEU | A | 382 | 36.849 | 36.750 | 55.764 | 1.00 | 28.01 | C |
| ATOM | 5700 | CG | LEU | A | 382 | 37.909 | 37.206 | 56.753 | 1.00 | 29.87 | C |
| ATOM | 5702 | CD1 | LEU | A | 382 | 37.249 | 38.057 | 57.829 | 1.00 | 31.23 | C |
| ATOM | 5706 | CD2 | LEU | A | 382 | 38.571 | 36.020 | 57.423 | 1.00 | 29.60 | C |
| ATOM | 5710 | C | LEU | A | 382 | 37.239 | 38.826 | 54.424 | 1.00 | 27.94 | C |
| ATOM | 5711 | O | LEU | A | 382 | 38.189 | 38.425 | 53.770 | 1.00 | 28.55 | O |
| ATOM | 5712 | N | ILE | A | 383 | 37.106 | 40.104 | 54.738 | 1.00 | 28.44 | N |
| ATOM | 5714 | CA | ILE | A | 383 | 37.992 | 41.136 | 54.168 | 1.00 | 29.01 | C |
| ATOM | 5716 | CB | ILE | A | 383 | 37.182 | 42.076 | 53.298 | 1.00 | 30.11 | C |
| ATOM | 5718 | CG1 | ILE | A | 383 | 36.362 | 41.266 | 52.311 | 1.00 | 31.00 | C |
| ATOM | 5721 | CD1 | ILE | A | 383 | 35.938 | 42.136 | 51.127 | 1.00 | 32.70 | C |
| ATOM | 5725 | CG2 | ILE | A | 383 | 38.119 | 43.072 | 52.508 | 1.00 | 29.84 | C |
| ATOM | 5729 | C | ILE | A | 383 | 38.695 | 41.952 | 55.210 | 1.00 | 28.25 | C |
| ATOM | 5730 | O | ILE | A | 383 | 38.130 | 42.865 | 55.743 | 1.00 | 27.90 | O |
| ATOM | 5731 | N | GLN | A | 384 | 39.944 | 41.635 | 55.486 | 1.00 | 28.72 | N |
| ATOM | 5733 | CA | GLN | A | 384 | 40.640 | 42.232 | 56.611 | 1.00 | 28.78 | C |
| ATOM | 5735 | CB | GLN | A | 384 | 41.213 | 41.148 | 57.429 | 1.00 | 28.34 | C |
| ATOM | 5738 | CG | GLN | A | 384 | 40.192 | 40.473 | 58.315 | 1.00 | 27.69 | C |
| ATOM | 5741 | CD | GLN | A | 384 | 40.787 | 39.489 | 59.310 | 1.00 | 25.79 | C |
| ATOM | 5742 | OE1 | GLN | A | 384 | 41.947 | 39.167 | 59.277 | 1.00 | 29.42 | O |
| ATOM | 5743 | NE2 | GLN | A | 384 | 39.997 | 39.070 | 60.220 | 1.00 | 29.85 | N |
| ATOM | 5746 | C | GLN | A | 384 | 41.735 | 43.122 | 56.073 | 1.00 | 30.69 | C |
| ATOM | 5747 | O | GLN | A | 384 | 42.402 | 43.817 | 56.826 | 1.00 | 33.98 | O |
| ATOM | 5748 | N | ALA | A | 385 | 41.892 | 43.164 | 54.756 | 1.00 | 30.13 | N |
| ATOM | 5750 | CA | ALA | A | 385 | 42.856 | 44.036 | 54.126 | 1.00 | 30.49 | C |
| ATOM | 5752 | CB | ALA | A | 385 | 44.242 | 43.332 | 54.044 | 1.00 | 30.62 | C |
| ATOM | 5756 | C | ALA | A | 385 | 42.348 | 44.372 | 52.728 | 1.00 | 31.09 | C |
| ATOM | 5757 | O | ALA | A | 385 | 41.586 | 43.598 | 52.083 | 1.00 | 30.22 | O |
| ATOM | 5758 | N | TRP | A | 386 | 42.808 | 45.504 | 52.232 | 1.00 | 32.17 | N |
| ATOM | 5760 | CA | TRP | A | 386 | 42.343 | 45.986 | 50.991 | 1.00 | 33.08 | C |
| ATOM | 5762 | CB | TRP | A | 386 | 40.903 | 46.412 | 51.231 | 1.00 | 32.74 | C |
| ATOM | 5765 | CG | TRP | A | 386 | 40.173 | 46.579 | 50.012 | 1.00 | 36.40 | C |
| ATOM | 5766 | CD1 | TRP | A | 386 | 39.885 | 47.756 | 49.354 | 1.00 | 40.08 | C |
| ATOM | 5768 | NE1 | TRP | A | 386 | 39.158 | 47.478 | 48.227 | 1.00 | 40.21 | N |
| ATOM | 5770 | CE2 | TRP | A | 386 | 39.049 | 46.114 | 48.114 | 1.00 | 40.12 | C |
| ATOM | 5771 | CD2 | TRP | A | 386 | 39.676 | 45.539 | 49.213 | 1.00 | 35.62 | C |
| ATOM | 5772 | CE3 | TRP | A | 386 | 39.657 | 44.174 | 49.339 | 1.00 | 34.88 | C |
| ATOM | 5774 | CZ3 | TRP | A | 386 | 39.049 | 43.446 | 48.400 | 1.00 | 37.08 | C |
| ATOM | 5776 | CH2 | TRP | A | 386 | 38.429 | 44.025 | 47.329 | 1.00 | 39.08 | C |
| ATOM | 5778 | CZ2 | TRP | A | 386 | 38.425 | 45.359 | 47.159 | 1.00 | 42.51 | C |
| ATOM | 5780 | C | TRP | A | 386 | 43.127 | 47.177 | 50.563 | 1.00 | 34.61 | C |
| ATOM | 5781 | O | TRP | A | 386 | 43.390 | 48.039 | 51.341 | 1.00 | 37.82 | O |
| ATOM | 5782 | N | PRO | A | 387 | 43.434 | 47.332 | 49.320 | 1.00 | 36.03 | N |
| ATOM | 5783 | CA | PRO | A | 387 | 44.281 | 48.435 | 48.949 | 1.00 | 38.07 | C |
| ATOM | 5785 | CB | PRO | A | 387 | 44.323 | 48.312 | 47.443 | 1.00 | 39.84 | C |
| ATOM | 5788 | CG | PRO | A | 387 | 43.252 | 47.516 | 47.081 | 1.00 | 38.50 | C |
| ATOM | 5791 | CD | PRO | A | 387 | 42.989 | 46.569 | 48.156 | 1.00 | 36.66 | C |
| ATOM | 5794 | C | PRO | A | 387 | 43.736 | 49.784 | 49.409 | 1.00 | 39.23 | C |
| ATOM | 5795 | O | PRO | A | 387 | 42.603 | 50.184 | 49.169 | 1.00 | 39.83 | O |
| ATOM | 5796 | N | GLU | A | 388 | 44.573 | 50.483 | 50.137 | 1.00 | 41.44 | N |
| ATOM | 5798 | CA | GLU | A | 388 | 44.233 | 51.765 | 50.763 | 1.00 | 43.29 | C |
| ATOM | 5800 | CB | GLU | A | 388 | 45.383 | 52.255 | 51.622 | 1.00 | 45.21 | C |
| ATOM | 5803 | CG | GLU | A | 388 | 45.567 | 51.443 | 52.897 | 1.00 | 45.28 | C |
| ATOM | 5806 | CD | GLU | A | 388 | 46.796 | 51.891 | 53.654 | 1.00 | 52.65 | C |
| ATOM | 5807 | OE1 | GLU | A | 388 | 47.893 | 51.297 | 53.459 | 1.00 | 53.52 | O |
| ATOM | 5808 | OE2 | GLU | A | 388 | 46.653 | 52.891 | 54.420 | 1.00 | 57.96 | O |
| ATOM | 5809 | C | GLU | A | 388 | 43.796 | 52.887 | 49.862 | 1.00 | 45.35 | C |
| ATOM | 5810 | O | GLU | A | 388 | 43.063 | 53.764 | 50.284 | 1.00 | 45.89 | O |
| ATOM | 5811 | N | ASN | A | 389 | 44.170 | 52.836 | 48.609 | 1.00 | 47.38 | N |
| ATOM | 5813 | CA | ASN | A | 389 | 43.678 | 53.842 | 47.663 | 1.00 | 50.51 | C |
| ATOM | 5815 | CB | ASN | A | 389 | 44.805 | 54.240 | 46.668 | 1.00 | 54.16 | C |
| ATOM | 5818 | CG | ASN | A | 389 | 45.254 | 53.045 | 45.796 | 1.00 | 56.14 | C |
| ATOM | 5819 | OD1 | ASN | A | 389 | 45.805 | 52.052 | 46.328 | 1.00 | 62.07 | O |
| ATOM | 5820 | ND2 | ASN | A | 389 | 44.886 | 53.055 | 44.521 | 1.00 | 57.83 | N |
| ATOM | 5823 | C | ASN | A | 389 | 42.455 | 53.391 | 46.860 | 1.00 | 49.44 | C |
| ATOM | 5824 | O | ASN | A | 389 | 42.303 | 53.859 | 45.756 | 1.00 | 52.95 | O |
| ATOM | 5825 | N | ARG | A | 390 | 41.611 | 52.494 | 47.344 | 1.00 | 46.09 | N |
| ATOM | 5827 | CA | ARG | A | 390 | 40.371 | 52.217 | 46.649 | 1.00 | 46.76 | C |
| ATOM | 5829 | CB | ARG | A | 390 | 40.234 | 50.742 | 46.244 | 1.00 | 45.10 | C |
| ATOM | 5832 | CG | ARG | A | 390 | 41.217 | 50.172 | 45.285 | 1.00 | 47.34 | C |
| ATOM | 5835 | CD | ARG | A | 390 | 41.438 | 50.956 | 43.977 | 1.00 | 53.54 | C |
| ATOM | 5838 | NE | ARG | A | 390 | 40.695 | 50.412 | 42.824 | 1.00 | 53.39 | N |
| ATOM | 5840 | CZ | ARG | A | 390 | 39.518 | 50.880 | 42.393 | 1.00 | 54.47 | C |
| ATOM | 5841 | NH1 | ARG | A | 390 | 38.862 | 51.878 | 43.014 | 1.00 | 59.31 | N |
| ATOM | 5844 | NH2 | ARG | A | 390 | 38.951 | 50.325 | 41.362 | 1.00 | 52.63 | N |
| ATOM | 5847 | C | ARG | A | 390 | 39.172 | 52.536 | 47.545 | 1.00 | 46.14 | C |
| ATOM | 5848 | O | ARG | A | 390 | 38.999 | 51.940 | 48.589 | 1.00 | 44.17 | O |
| ATOM | 5849 | N | THR | A | 391 | 38.283 | 53.411 | 47.114 | 1.00 | 48.34 | N |
| ATOM | 5851 | CA | THR | A | 391 | 37.246 | 53.899 | 48.039 | 1.00 | 47.56 | C |
| ATOM | 5853 | CB | THR | A | 391 | 36.645 | 55.201 | 47.543 | 1.00 | 50.22 | C |
| ATOM | 5855 | OG1 | THR | A | 391 | 35.870 | 54.878 | 46.403 | 1.00 | 51.02 | O |
| ATOM | 5857 | CG2 | THR | A | 391 | 37.689 | 56.203 | 47.035 | 1.00 | 51.77 | C |
| ATOM | 5861 | C | THR | A | 391 | 36.102 | 52.893 | 48.239 | 1.00 | 45.74 | C |
| ATOM | 5862 | O | THR | A | 391 | 35.269 | 53.087 | 49.121 | 1.00 | 45.03 | O |
| ATOM | 5863 | N | ASP | A | 392 | 36.052 | 51.812 | 47.467 | 1.00 | 44.71 | N |
| ATOM | 5865 | CA | ASP | A | 392 | 35.129 | 50.743 | 47.833 | 1.00 | 42.13 | C |
| ATOM | 5867 | CB | ASP | A | 392 | 33.840 | 50.977 | 47.144 | 1.00 | 43.91 | C |
| ATOM | 5870 | CG | ASP | A | 392 | 33.994 | 51.000 | 45.722 | 1.00 | 45.23 | C |
| ATOM | 5871 | OD1 | ASP | A | 392 | 35.000 | 50.496 | 45.229 | 1.00 | 45.27 | O |
| ATOM | 5872 | OD2 | ASP | A | 392 | 33.161 | 51.519 | 45.017 | 1.00 | 46.76 | O |
| ATOM | 5873 | C | ASP | A | 392 | 35.663 | 49.385 | 47.506 | 1.00 | 40.51 | C |
| ATOM | 5874 | O | ASP | A | 392 | 36.837 | 49.268 | 47.180 | 1.00 | 42.21 | O |
| ATOM | 5875 | N | LEU | A | 393 | 34.839 | 48.351 | 47.691 | 1.00 | 39.13 | N |
| ATOM | 5877 | CA | LEU | A | 393 | 35.208 | 46.964 | 47.389 | 1.00 | 37.38 | C |
| ATOM | 5879 | CB | LEU | A | 393 | 34.402 | 45.992 | 48.238 | 1.00 | 35.46 | C |
| ATOM | 5882 | CG | LEU | A | 393 | 34.425 | 46.226 | 49.733 | 1.00 | 34.54 | C |
| ATOM | 5884 | CD1 | LEU | A | 393 | 33.565 | 45.205 | 50.454 | 1.00 | 34.20 | C |
| ATOM | 5888 | CD2 | LEU | A | 393 | 35.810 | 46.075 | 50.173 | 1.00 | 34.63 | C |
| ATOM | 5892 | C | LEU | A | 393 | 34.967 | 46.666 | 45.901 | 1.00 | 38.22 | C |
| ATOM | 5893 | O | LEU | A | 393 | 34.103 | 45.865 | 45.572 | 1.00 | 38.13 | O |
| ATOM | 5894 | N | HIS | A | 394 | 35.752 | 47.300 | 45.025 | 1.00 | 38.65 | N |
| ATOM | 5896 | CA | HIS | A | 394 | 35.460 | 47.287 | 43.607 | 1.00 | 40.24 | C |
| ATOM | 5898 | CB | HIS | A | 394 | 36.506 | 48.073 | 42.878 | 1.00 | 41.20 | C |
| ATOM | 5901 | CG | HIS | A | 394 | 37.873 | 47.539 | 43.061 | 1.00 | 43.28 | C |
| ATOM | 5902 | ND1 | HIS | A | 394 | 38.577 | 47.684 | 44.242 | 1.00 | 44.16 | N |
| ATOM | 5904 | CE1 | HIS | A | 394 | 39.754 | 47.091 | 44.108 | 1.00 | 44.04 | C |
| ATOM | 5906 | NE2 | HIS | A | 394 | 39.820 | 46.535 | 42.906 | 1.00 | 44.28 | N |
| ATOM | 5908 | CD2 | HIS | A | 394 | 38.649 | 46.787 | 42.240 | 1.00 | 44.56 | C |
| ATOM | 5910 | C | HIS | A | 394 | 35.397 | 45.864 | 43.053 | 1.00 | 39.79 | C |
| ATOM | 5911 | O | HIS | A | 394 | 34.658 | 45.565 | 42.118 | 1.00 | 39.77 | O |
| ATOM | 5912 | N | ALA | A | 395 | 36.186 | 44.971 | 43.639 | 1.00 | 38.62 | N |
| ATOM | 5914 | CA | ALA | A | 395 | 36.342 | 43.675 | 43.038 | 1.00 | 38.76 | C |
| ATOM | 5916 | CB | ALA | A | 395 | 37.493 | 43.036 | 43.581 | 1.00 | 37.86 | C |
| ATOM | 5920 | C | ALA | A | 395 | 35.102 | 42.811 | 43.228 | 1.00 | 38.35 | C |
| ATOM | 5921 | O | ALA | A | 395 | 34.964 | 41.819 | 42.513 | 1.00 | 39.40 | O |
| ATOM | 5922 | N | PHE | A | 396 | 34.194 | 43.239 | 44.111 | 1.00 | 36.67 | N |
| ATOM | 5924 | CA | PHE | A | 396 | 32.986 | 42.520 | 44.416 | 1.00 | 36.80 | C |
| ATOM | 5926 | CB | PHE | A | 396 | 32.934 | 42.244 | 45.922 | 1.00 | 35.11 | C |
| ATOM | 5929 | CG | PHE | A | 396 | 34.060 | 41.284 | 46.399 | 1.00 | 34.66 | C |
| ATOM | 5930 | CD1 | PHE | A | 396 | 35.141 | 41.734 | 47.116 | 1.00 | 31.86 | C |
| ATOM | 5932 | CE1 | PHE | A | 396 | 36.149 | 40.871 | 47.508 | 1.00 | 30.93 | C |
| ATOM | 5934 | CZ | PHE | A | 396 | 36.112 | 39.574 | 47.201 | 1.00 | 31.65 | C |
| ATOM | 5936 | CE2 | PHE | A | 396 | 35.056 | 39.084 | 46.528 | 1.00 | 33.87 | C |
| ATOM | 5938 | CD2 | PHE | A | 396 | 34.020 | 39.942 | 46.097 | 1.00 | 34.52 | C |
| ATOM | 5940 | C | PHE | A | 396 | 31.709 | 43.215 | 43.939 | 1.00 | 38.98 | C |
| ATOM | 5941 | O | PHE | A | 396 | 30.598 | 42.884 | 44.347 | 1.00 | 40.05 | O |
| ATOM | 5942 | N | GLU | A | 397 | 31.828 | 44.157 | 43.030 | 1.00 | 41.06 | N |
| ATOM | 5944 | CA | GLU | A | 397 | 30.664 | 44.849 | 42.495 | 1.00 | 43.09 | C |
| ATOM | 5946 | CB | GLU | A | 397 | 31.192 | 45.871 | 41.507 | 1.00 | 45.95 | C |
| ATOM | 5949 | CG | GLU | A | 397 | 31.738 | 45.211 | 40.255 | 1.00 | 49.91 | C |
| ATOM | 5952 | CD | GLU | A | 397 | 32.326 | 46.179 | 39.236 | 1.00 | 58.00 | C |
| ATOM | 5953 | OE1 | GLU | A | 397 | 31.536 | 46.971 | 38.642 | 1.00 | 63.84 | O |
| ATOM | 5954 | OE2 | GLU | A | 397 | 33.574 | 46.098 | 38.994 | 1.00 | 59.87 | O |
| ATOM | 5955 | C | GLU | A | 397 | 29.588 | 43.961 | 41.771 | 1.00 | 44.36 | C |
| ATOM | 5956 | O | GLU | A | 397 | 28.513 | 44.449 | 41.344 | 1.00 | 45.29 | O |
| ATOM | 5957 | N | ASN | A | 398 | 29.882 | 42.681 | 41.554 | 1.00 | 43.83 | N |
| ATOM | 5959 | CA | ASN | A | 398 | 28.902 | 41.797 | 40.905 | 1.00 | 44.75 | C |
| ATOM | 5961 | CB | ASN | A | 398 | 29.486 | 41.235 | 39.638 | 1.00 | 46.12 | C |
| ATOM | 5964 | CG | ASN | A | 398 | 29.691 | 42.290 | 38.613 | 1.00 | 46.75 | C |
| ATOM | 5965 | OD1 | ASN | A | 398 | 28.812 | 43.035 | 38.309 | 1.00 | 50.16 | O |
| ATOM | 5966 | ND2 | ASN | A | 398 | 30.866 | 42.371 | 38.097 | 1.00 | 47.71 | N |
| ATOM | 5969 | C | ASN | A | 398 | 28.408 | 40.673 | 41.794 | 1.00 | 43.09 | C |
| ATOM | 5970 | O | ASN | A | 398 | 27.632 | 39.847 | 41.387 | 1.00 | 44.84 | O |
| ATOM | 5971 | N | LEU | A | 399 | 28.830 | 40.707 | 43.033 | 1.00 | 40.07 | N |
| ATOM | 5973 | CA | LEU | A | 399 | 28.488 | 39.731 | 43.975 | 1.00 | 38.64 | C |
| ATOM | 5975 | CB | LEU | A | 399 | 29.387 | 39.940 | 45.179 | 1.00 | 35.59 | C |
| ATOM | 5978 | CG | LEU | A | 399 | 29.067 | 39.022 | 46.299 | 1.00 | 34.56 | C |
| ATOM | 5980 | CD1 | LEU | A | 399 | 29.351 | 37.598 | 45.883 | 1.00 | 36.89 | C |
| ATOM | 5984 | CD2 | LEU | A | 399 | 29.858 | 39.414 | 47.436 | 1.00 | 34.33 | C |
| ATOM | 5988 | C | LEU | A | 399 | 27.014 | 39.935 | 44.321 | 1.00 | 41.17 | C |
| ATOM | 5989 | O | LEU | A | 399 | 26.567 | 40.998 | 44.836 | 1.00 | 42.73 | O |
| ATOM | 5990 | N | GLU | A | 400 | 26.261 | 38.884 | 44.057 | 1.00 | 42.86 | N |
| ATOM | 5992 | CA | GLU | A | 400 | 24.817 | 38.875 | 44.151 | 1.00 | 44.43 | C |
| ATOM | 5994 | CB | GLU | A | 400 | 24.277 | 38.174 | 42.923 | 1.00 | 46.71 | C |
| ATOM | 5997 | CG | GLU | A | 400 | 24.484 | 39.044 | 41.719 | 1.00 | 50.19 | C |
| ATOM | 6000 | CD | GLU | A | 400 | 24.161 | 38.401 | 40.366 | 1.00 | 56.20 | C |
| ATOM | 6001 | OE1 | GLU | A | 400 | 23.095 | 37.786 | 40.242 | 1.00 | 56.23 | O |
| ATOM | 6002 | OE2 | GLU | A | 400 | 24.980 | 38.579 | 39.408 | 1.00 | 58.34 | O |
| ATOM | 6003 | C | GLU | A | 400 | 24.316 | 38.192 | 45.421 | 1.00 | 43.67 | C |
| ATOM | 6004 | O | GLU | A | 400 | 23.233 | 38.518 | 45.885 | 1.00 | 44.99 | O |
| ATOM | 6005 | N | ILE | A | 401 | 25.130 | 37.306 | 46.011 | 1.00 | 41.83 | N |
| ATOM | 6007 | CA | ILE | A | 401 | 24.737 | 36.490 | 47.162 | 1.00 | 40.84 | C |
| ATOM | 6009 | CB | ILE | A | 401 | 23.734 | 35.422 | 46.710 | 1.00 | 42.90 | C |
| ATOM | 6011 | CG1 | ILE | A | 401 | 23.449 | 34.429 | 47.814 | 1.00 | 43.25 | C |
| ATOM | 6014 | CD1 | ILE | A | 401 | 22.256 | 33.586 | 47.448 | 1.00 | 45.27 | C |
| ATOM | 6018 | CG2 | ILE | A | 401 | 24.253 | 34.643 | 45.619 | 1.00 | 44.73 | C |
| ATOM | 6022 | C | ILE | A | 401 | 25.897 | 35.831 | 47.895 | 1.00 | 38.33 | C |
| ATOM | 6023 | O | ILE | A | 401 | 26.794 | 35.247 | 47.303 | 1.00 | 37.78 | O |
| ATOM | 6024 | N | ILE | A | 402 | 25.857 | 35.927 | 49.206 | 1.00 | 37.64 | N |
| ATOM | 6026 | CA | ILE | A | 402 | 26.758 | 35.219 | 50.099 | 1.00 | 35.89 | C |
| ATOM | 6028 | CB | ILE | A | 402 | 27.313 | 36.190 | 51.117 | 1.00 | 33.76 | C |
| ATOM | 6030 | CG1 | ILE | A | 402 | 28.327 | 37.122 | 50.428 | 1.00 | 33.45 | C |
| ATOM | 6033 | CD1 | ILE | A | 402 | 28.729 | 38.356 | 51.238 | 1.00 | 30.73 | C |
| ATOM | 6037 | CG2 | ILE | A | 402 | 27.959 | 35.445 | 52.248 | 1.00 | 33.22 | C |
| ATOM | 6041 | C | ILE | A | 402 | 25.872 | 34.221 | 50.789 | 1.00 | 38.27 | C |
| ATOM | 6042 | O | ILE | A | 402 | 25.042 | 34.590 | 51.611 | 1.00 | 40.21 | O |
| ATOM | 6043 | N | ARG | A | 403 | 25.961 | 32.954 | 50.440 | 1.00 | 39.72 | N |
| ATOM | 6045 | CA | ARG | A | 403 | 25.029 | 31.971 | 51.036 | 1.00 | 41.41 | C |
| ATOM | 6047 | CB | ARG | A | 403 | 25.094 | 30.665 | 50.280 | 1.00 | 42.06 | C |
| ATOM | 6050 | CG | ARG | A | 403 | 24.566 | 30.759 | 48.913 | 1.00 | 42.50 | C |
| ATOM | 6053 | CD | ARG | A | 403 | 24.271 | 29.361 | 48.306 | 1.00 | 45.57 | C |
| ATOM | 6056 | NE | ARG | A | 403 | 23.861 | 29.447 | 46.910 | 1.00 | 46.93 | N |
| ATOM | 6058 | CZ | ARG | A | 403 | 22.741 | 30.008 | 46.512 | 1.00 | 49.93 | C |
| ATOM | 6059 | NH1 | ARG | A | 403 | 21.876 | 30.503 | 47.387 | 1.00 | 52.29 | N |
| ATOM | 6062 | NH2 | ARG | A | 403 | 22.461 | 30.068 | 45.235 | 1.00 | 52.68 | N |
| ATOM | 6065 | C | ARG | A | 403 | 25.272 | 31.712 | 52.541 | 1.00 | 40.44 | C |
| ATOM | 6066 | O | ARG | A | 403 | 24.332 | 31.432 | 53.262 | 1.00 | 41.87 | O |
| ATOM | 6067 | N | GLY | A | 404 | 26.520 | 31.772 | 52.979 | 1.00 | 38.47 | N |
| ATOM | 6069 | CA | GLY | A | 404 | 26.841 | 31.694 | 54.394 | 1.00 | 38.72 | C |
| ATOM | 6072 | C | GLY | A | 404 | 26.779 | 30.296 | 55.021 | 1.00 | 40.86 | C |
| ATOM | 6073 | O | GLY | A | 404 | 26.528 | 30.097 | 56.219 | 1.00 | 42.25 | O |
| ATOM | 6074 | N | ARG | A | 405 | 26.982 | 29.284 | 54.204 | 1.00 | 41.64 | N |
| ATOM | 6076 | CA | ARG | A | 405 | 26.955 | 27.903 | 54.687 | 1.00 | 43.01 | C |
| ATOM | 6078 | CB | ARG | A | 405 | 27.068 | 27.061 | 53.455 | 1.00 | 44.80 | C |
| ATOM | 6081 | CG | ARG | A | 405 | 25.790 | 26.999 | 52.727 | 1.00 | 46.83 | C |
| ATOM | 6084 | CD | ARG | A | 405 | 25.923 | 26.159 | 51.460 | 1.00 | 49.83 | C |
| ATOM | 6087 | NE | ARG | A | 405 | 24.656 | 26.202 | 50.690 | 1.00 | 54.46 | N |
| ATOM | 6089 | CZ | ARG | A | 405 | 24.534 | 25.597 | 49.539 | 1.00 | 55.52 | C |
| ATOM | 6090 | NH1 | ARG | A | 405 | 25.589 | 24.930 | 49.073 | 1.00 | 55.71 | N |
| ATOM | 6093 | NH2 | ARG | A | 405 | 23.379 | 25.608 | 48.890 | 1.00 | 59.28 | N |
| ATOM | 6096 | C | ARG | A | 405 | 28.043 | 27.497 | 55.659 | 1.00 | 40.66 | C |
| ATOM | 6097 | O | ARG | A | 405 | 27.881 | 26.744 | 56.531 | 1.00 | 41.11 | O |
| ATOM | 6098 | N | THR | A | 406 | 29.194 | 27.962 | 55.351 | 1.00 | 40.05 | N |
| ATOM | 6100 | CA | THR | A | 406 | 30.381 | 28.035 | 56.186 | 1.00 | 40.02 | C |
| ATOM | 6102 | CB | THR | A | 406 | 31.557 | 27.731 | 55.244 | 1.00 | 39.43 | C |
| ATOM | 6104 | OG1 | THR | A | 406 | 31.310 | 26.463 | 54.715 | 1.00 | 43.84 | O |
| ATOM | 6106 | CG2 | THR | A | 406 | 32.910 | 27.424 | 55.938 | 1.00 | 43.39 | C |
| ATOM | 6110 | C | THR | A | 406 | 30.393 | 29.475 | 56.679 | 1.00 | 37.35 | C |
| ATOM | 6111 | O | THR | A | 406 | 29.970 | 30.388 | 55.965 | 1.00 | 36.01 | O |
| ATOM | 6112 | N | LYS | A | 407 | 30.744 | 29.669 | 57.936 | 1.00 | 38.03 | N |
| ATOM | 6114 | CA | LYS | A | 407 | 30.913 | 31.032 | 58.480 | 1.00 | 37.24 | C |
| ATOM | 6116 | CB | LYS | A | 407 | 29.808 | 31.396 | 59.427 | 1.00 | 38.61 | C |
| ATOM | 6119 | CG | LYS | A | 407 | 28.408 | 31.305 | 58.843 | 1.00 | 39.27 | C |
| ATOM | 6122 | CD | LYS | A | 407 | 27.467 | 30.706 | 59.864 | 1.00 | 41.05 | C |
| ATOM | 6125 | CE | LYS | A | 407 | 26.150 | 30.400 | 59.316 | 1.00 | 44.32 | C |
| ATOM | 6128 | NZ | LYS | A | 407 | 25.229 | 31.288 | 60.019 | 1.00 | 52.24 | N |
| ATOM | 6132 | C | LYS | A | 407 | 32.218 | 31.114 | 59.237 | 1.00 | 37.24 | C |
| ATOM | 6133 | O | LYS | A | 407 | 32.849 | 30.135 | 59.489 | 1.00 | 40.60 | O |
| ATOM | 6134 | N | GLN | A | 408 | 32.674 | 32.299 | 59.552 | 1.00 | 36.38 | N |
| ATOM | 6136 | CA | GLN | A | 408 | 33.979 | 32.465 | 60.158 | 1.00 | 34.55 | C |
| ATOM | 6138 | CB | GLN | A | 408 | 34.581 | 33.801 | 59.825 | 1.00 | 32.09 | C |
| ATOM | 6141 | CG | GLN | A | 408 | 35.948 | 33.899 | 60.498 | 1.00 | 31.64 | C |
| ATOM | 6144 | CD | GLN | A | 408 | 36.473 | 35.248 | 60.620 | 1.00 | 30.23 | C |
| ATOM | 6145 | OE1 | GLN | A | 408 | 35.772 | 36.221 | 60.467 | 1.00 | 39.71 | O |
| ATOM | 6146 | NE2 | GLN | A | 408 | 37.680 | 35.336 | 60.852 | 1.00 | 26.16 | N |
| ATOM | 6149 | C | GLN | A | 408 | 33.734 | 32.402 | 61.666 | 1.00 | 35.59 | C |
| ATOM | 6150 | O | GLN | A | 408 | 32.869 | 33.106 | 62.167 | 1.00 | 34.95 | O |
| ATOM | 6151 | N | HIS | A | 409 | 34.501 | 31.551 | 62.338 | 1.00 | 35.68 | N |
| ATOM | 6153 | CA | HIS | A | 409 | 34.305 | 31.239 | 63.709 | 1.00 | 36.96 | C |
| ATOM | 6155 | CB | HIS | A | 409 | 34.594 | 32.495 | 64.525 | 1.00 | 36.30 | C |
| ATOM | 6158 | CG | HIS | A | 409 | 35.974 | 32.999 | 64.388 | 1.00 | 32.03 | C |
| ATOM | 6159 | ND1 | HIS | A | 409 | 37.046 | 32.335 | 64.885 | 1.00 | 36.03 | N |
| ATOM | 6161 | CE1 | HIS | A | 409 | 38.149 | 32.995 | 64.602 | 1.00 | 32.96 | C |
| ATOM | 6163 | NE2 | HIS | A | 409 | 37.823 | 34.099 | 63.993 | 1.00 | 29.44 | N |
| ATOM | 6165 | CD2 | HIS | A | 409 | 36.464 | 34.108 | 63.819 | 1.00 | 33.90 | C |
| ATOM | 6167 | C | HIS | A | 409 | 32.898 | 30.688 | 63.922 | 1.00 | 38.45 | C |
| ATOM | 6168 | O | HIS | A | 409 | 32.282 | 30.785 | 64.967 | 1.00 | 39.82 | O |
| ATOM | 6169 | N | GLY | A | 410 | 32.341 | 30.135 | 62.879 | 1.00 | 39.18 | N |
| ATOM | 6171 | CA | GLY | A | 410 | 30.948 | 29.727 | 62.974 | 1.00 | 40.64 | C |
| ATOM | 6174 | C | GLY | A | 410 | 30.003 | 30.872 | 63.123 | 1.00 | 39.89 | C |
| ATOM | 6175 | O | GLY | A | 410 | 28.908 | 30.596 | 63.288 | 1.00 | 40.91 | O |
| ATOM | 6176 | N | ASN | A | 411 | 30.443 | 32.129 | 63.096 | 1.00 | 39.05 | N |
| ATOM | 6178 | CA | ASN | A | 411 | 29.582 | 33.303 | 63.276 | 1.00 | 39.30 | C |
| ATOM | 6180 | CB | ASN | A | 411 | 30.176 | 34.429 | 64.213 | 1.00 | 39.22 | C |
| ATOM | 6183 | CG | ASN | A | 411 | 30.202 | 34.049 | 65.693 | 1.00 | 44.51 | C |
| ATOM | 6184 | OD1 | ASN | A | 411 | 29.929 | 32.909 | 65.994 | 1.00 | 52.63 | O |
| ATOM | 6185 | ND2 | ASN | A | 411 | 30.548 | 34.986 | 66.614 | 1.00 | 41.99 | N |
| ATOM | 6188 | C | ASN | A | 411 | 29.312 | 33.940 | 61.907 | 1.00 | 37.22 | C |
| ATOM | 6189 | O | ASN | A | 411 | 28.194 | 33.948 | 61.435 | 1.00 | 37.23 | O |
| ATOM | 6190 | N | PHE | A | 412 | 30.342 | 34.474 | 61.278 | 1.00 | 34.68 | N |
| ATOM | 6192 | CA | PHE | A | 412 | 30.135 | 35.478 | 60.287 | 1.00 | 33.77 | C |
| ATOM | 6194 | CB | PHE | A | 412 | 31.294 | 36.438 | 60.393 | 1.00 | 32.92 | C |
| ATOM | 6197 | CG | PHE | A | 412 | 31.440 | 37.028 | 61.750 | 1.00 | 33.90 | C |
| ATOM | 6198 | CD1 | PHE | A | 412 | 32.607 | 36.831 | 62.507 | 1.00 | 34.35 | C |
| ATOM | 6200 | CE1 | PHE | A | 412 | 32.708 | 37.353 | 63.793 | 1.00 | 31.85 | C |
| ATOM | 6202 | CZ | PHE | A | 412 | 31.688 | 38.084 | 64.275 | 1.00 | 33.72 | C |
| ATOM | 6204 | CE2 | PHE | A | 412 | 30.575 | 38.290 | 63.552 | 1.00 | 33.28 | C |
| ATOM | 6206 | CD2 | PHE | A | 412 | 30.419 | 37.748 | 62.306 | 1.00 | 32.58 | C |
| ATOM | 6208 | C | PHE | A | 412 | 30.053 | 34.983 | 58.856 | 1.00 | 33.95 | C |
| ATOM | 6209 | O | PHE | A | 412 | 30.865 | 34.164 | 58.410 | 1.00 | 34.69 | O |
| ATOM | 6210 | N | SER | A | 413 | 29.101 | 35.512 | 58.088 | 1.00 | 34.35 | N |
| ATOM | 6212 | CA | SER | A | 413 | 29.154 | 35.319 | 56.617 | 1.00 | 33.33 | C |
| ATOM | 6214 | CB | SER | A | 413 | 27.803 | 34.919 | 56.082 | 1.00 | 34.29 | C |
| ATOM | 6217 | OG | SER | A | 413 | 26.927 | 35.986 | 56.195 | 1.00 | 34.47 | O |
| ATOM | 6219 | C | SER | A | 413 | 29.711 | 36.474 | 55.800 | 1.00 | 31.62 | C |
| ATOM | 6220 | O | SER | A | 413 | 29.980 | 36.268 | 54.679 | 1.00 | 30.19 | O |
| ATOM | 6221 | N | LEU | A | 414 | 29.810 | 37.698 | 56.361 | 1.00 | 32.41 | N |
| ATOM | 6223 | CA | LEU | A | 414 | 30.474 | 38.882 | 55.716 | 1.00 | 31.11 | C |
| ATOM | 6225 | CB | LEU | A | 414 | 29.458 | 39.830 | 55.119 | 1.00 | 32.13 | C |
| ATOM | 6228 | CG | LEU | A | 414 | 29.895 | 41.226 | 54.590 | 1.00 | 33.57 | C |
| ATOM | 6230 | CD1 | LEU | A | 414 | 31.044 | 41.176 | 53.529 | 1.00 | 33.54 | C |
| ATOM | 6234 | CD2 | LEU | A | 414 | 28.737 | 41.998 | 53.991 | 1.00 | 34.32 | C |
| ATOM | 6238 | C | LEU | A | 414 | 31.241 | 39.660 | 56.755 | 1.00 | 30.16 | C |
| ATOM | 6239 | O | LEU | A | 414 | 30.616 | 40.240 | 57.603 | 1.00 | 31.21 | O |
| ATOM | 6240 | N | ALA | A | 415 | 32.565 | 39.668 | 56.721 | 1.00 | 28.59 | N |
| ATOM | 6242 | CA | ALA | A | 415 | 33.347 | 40.421 | 57.709 | 1.00 | 28.16 | C |
| ATOM | 6244 | CB | ALA | A | 415 | 34.171 | 39.540 | 58.506 | 1.00 | 27.51 | C |
| ATOM | 6248 | C | ALA | A | 415 | 34.233 | 41.399 | 56.975 | 1.00 | 28.12 | C |
| ATOM | 6249 | O | ALA | A | 415 | 34.998 | 41.005 | 56.099 | 1.00 | 28.60 | O |
| ATOM | 6250 | N | VAL | A | 416 | 34.077 | 42.689 | 57.294 | 1.00 | 28.48 | N |
| ATOM | 6252 | CA | VAL | A | 416 | 34.865 | 43.768 | 56.701 | 1.00 | 27.99 | C |
| ATOM | 6254 | CB | VAL | A | 416 | 33.940 | 44.600 | 55.867 | 1.00 | 28.89 | C |
| ATOM | 6256 | CG1 | VAL | A | 416 | 34.667 | 45.745 | 55.232 | 1.00 | 29.37 | C |
| ATOM | 6260 | CG2 | VAL | A | 416 | 33.294 | 43.709 | 54.828 | 1.00 | 29.55 | C |
| ATOM | 6264 | C | VAL | A | 416 | 35.492 | 44.588 | 57.822 | 1.00 | 27.40 | C |
| ATOM | 6265 | O | VAL | A | 416 | 34.788 | 45.252 | 58.514 | 1.00 | 28.63 | O |
| ATOM | 6266 | N | VAL | A | 417 | 36.805 | 44.579 | 57.982 | 1.00 | 26.45 | N |
| ATOM | 6268 | CA | VAL | A | 417 | 37.390 | 45.086 | 59.181 | 1.00 | 27.04 | C |
| ATOM | 6270 | CB | VAL | A | 417 | 37.707 | 43.940 | 60.175 | 1.00 | 27.03 | C |
| ATOM | 6272 | CG1 | VAL | A | 417 | 38.177 | 44.508 | 61.435 | 1.00 | 31.02 | C |
| ATOM | 6276 | CG2 | VAL | A | 417 | 36.616 | 43.085 | 60.494 | 1.00 | 25.94 | C |
| ATOM | 6280 | C | VAL | A | 417 | 38.700 | 45.849 | 58.959 | 1.00 | 27.79 | C |
| ATOM | 6281 | O | VAL | A | 417 | 39.729 | 45.304 | 58.592 | 1.00 | 28.44 | O |
| ATOM | 6282 | N | SER | A | 418 | 38.659 | 47.118 | 59.304 | 1.00 | 29.69 | N |
| ATOM | 6284 | CA | SER | A | 418 | 39.783 | 48.037 | 59.273 | 1.00 | 29.96 | C |
| ATOM | 6286 | CB | SER | A | 418 | 40.834 | 47.559 | 60.216 | 1.00 | 30.43 | C |
| ATOM | 6289 | OG | SER | A | 418 | 41.770 | 48.555 | 60.517 | 1.00 | 34.34 | O |
| ATOM | 6291 | C | SER | A | 418 | 40.320 | 48.213 | 57.889 | 1.00 | 30.06 | C |
| ATOM | 6292 | O | SER | A | 418 | 41.441 | 48.007 | 57.668 | 1.00 | 30.27 | O |
| ATOM | 6293 | N | LEU | A | 419 | 39.473 | 48.581 | 56.934 | 1.00 | 30.95 | N |
| ATOM | 6295 | CA | LEU | A | 419 | 39.881 | 49.016 | 55.578 | 1.00 | 30.62 | C |
| ATOM | 6297 | CB | LEU | A | 419 | 38.974 | 48.380 | 54.560 | 1.00 | 29.76 | C |
| ATOM | 6300 | CG | LEU | A | 419 | 38.564 | 46.915 | 54.692 | 1.00 | 27.89 | C |
| ATOM | 6302 | CD1 | LEU | A | 419 | 37.844 | 46.349 | 53.455 | 1.00 | 26.37 | C |
| ATOM | 6306 | CD2 | LEU | A | 419 | 39.796 | 46.136 | 54.942 | 1.00 | 30.27 | C |
| ATOM | 6310 | C | LEU | A | 419 | 39.787 | 50.520 | 55.513 | 1.00 | 31.84 | C |
| ATOM | 6311 | O | LEU | A | 419 | 39.248 | 51.129 | 56.415 | 1.00 | 34.04 | O |
| ATOM | 6312 | N | ASN | A | 420 | 40.347 | 51.141 | 54.505 | 1.00 | 32.95 | N |
| ATOM | 6314 | CA | ASN | A | 420 | 40.409 | 52.602 | 54.421 | 1.00 | 35.62 | C |
| ATOM | 6316 | CB | ASN | A | 420 | 41.682 | 53.008 | 53.739 | 1.00 | 37.74 | C |
| ATOM | 6319 | CG | ASN | A | 420 | 41.961 | 54.452 | 53.815 | 1.00 | 40.33 | C |
| ATOM | 6320 | OD1 | ASN | A | 420 | 41.713 | 55.072 | 54.796 | 1.00 | 41.29 | O |
| ATOM | 6321 | ND2 | ASN | A | 420 | 42.543 | 54.991 | 52.774 | 1.00 | 42.87 | N |
| ATOM | 6324 | C | ASN | A | 420 | 39.322 | 53.016 | 53.519 | 1.00 | 38.01 | C |
| ATOM | 6325 | O | ASN | A | 420 | 39.319 | 54.112 | 53.010 | 1.00 | 41.16 | O |
| ATOM | 6326 | N | ILE | A | 421 | 38.383 | 52.103 | 53.301 | 1.00 | 37.63 | N |
| ATOM | 6328 | CA | ILE | A | 421 | 37.280 | 52.252 | 52.393 | 1.00 | 38.22 | C |
| ATOM | 6330 | CB | ILE | A | 421 | 36.767 | 50.873 | 52.179 | 1.00 | 36.26 | C |
| ATOM | 6332 | CG1 | ILE | A | 421 | 36.784 | 50.662 | 50.724 | 1.00 | 40.93 | C |
| ATOM | 6335 | CD1 | ILE | A | 421 | 36.644 | 49.237 | 50.423 | 1.00 | 45.47 | C |
| ATOM | 6339 | CG2 | ILE | A | 421 | 35.404 | 50.467 | 52.732 | 1.00 | 34.04 | C |
| ATOM | 6343 | C | ILE | A | 421 | 36.263 | 53.290 | 52.797 | 1.00 | 40.58 | C |
| ATOM | 6344 | O | ILE | A | 421 | 36.203 | 53.706 | 53.954 | 1.00 | 42.88 | O |
| ATOM | 6345 | N | THR | A | 422 | 35.511 | 53.755 | 51.814 | 1.00 | 42.92 | N |
| ATOM | 6347 | CA | THR | A | 422 | 34.541 | 54.868 | 51.949 | 1.00 | 44.98 | C |
| ATOM | 6349 | CB | THR | A | 422 | 34.870 | 55.912 | 50.914 | 1.00 | 47.73 | C |
| ATOM | 6351 | OG1 | THR | A | 422 | 36.200 | 56.430 | 51.181 | 1.00 | 49.14 | O |
| ATOM | 6353 | CG2 | THR | A | 422 | 33.950 | 57.054 | 51.018 | 1.00 | 49.95 | C |
| ATOM | 6357 | C | THR | A | 422 | 33.106 | 54.418 | 51.763 | 1.00 | 44.41 | C |
| ATOM | 6358 | O | THR | A | 422 | 32.230 | 54.898 | 52.433 | 1.00 | 46.45 | O |
| ATOM | 6359 | N | SER | A | 423 | 32.878 | 53.462 | 50.889 | 1.00 | 42.64 | N |
| ATOM | 6361 | CA | SER | A | 423 | 31.626 | 52.762 | 50.831 | 1.00 | 41.79 | C |
| ATOM | 6363 | CB | SER | A | 423 | 30.874 | 53.300 | 49.666 | 1.00 | 44.68 | C |
| ATOM | 6366 | OG | SER | A | 423 | 31.655 | 53.146 | 48.490 | 1.00 | 46.25 | O |
| ATOM | 6368 | C | SER | A | 423 | 31.838 | 51.294 | 50.560 | 1.00 | 38.94 | C |
| ATOM | 6369 | O | SER | A | 423 | 32.862 | 50.907 | 50.044 | 1.00 | 37.59 | O |
| ATOM | 6370 | N | LEU | A | 424 | 30.846 | 50.468 | 50.858 | 1.00 | 38.17 | N |
| ATOM | 6372 | CA | LEU | A | 424 | 31.038 | 49.034 | 50.632 | 1.00 | 36.68 | C |
| ATOM | 6374 | CB | LEU | A | 424 | 29.949 | 48.233 | 51.394 | 1.00 | 35.68 | C |
| ATOM | 6377 | CG | LEU | A | 424 | 30.177 | 48.127 | 52.916 | 1.00 | 36.14 | C |
| ATOM | 6379 | CD1 | LEU | A | 424 | 28.913 | 47.658 | 53.776 | 1.00 | 34.01 | C |
| ATOM | 6383 | CD2 | LEU | A | 424 | 31.441 | 47.302 | 53.250 | 1.00 | 33.86 | C |
| ATOM | 6387 | C | LEU | A | 424 | 31.090 | 48.724 | 49.078 | 1.00 | 36.95 | C |
| ATOM | 6388 | O | LEU | A | 424 | 31.909 | 48.010 | 48.555 | 1.00 | 33.92 | O |
| ATOM | 6389 | N | GLY | A | 425 | 30.182 | 49.314 | 48.364 | 1.00 | 38.75 | N |
| ATOM | 6391 | CA | GLY | A | 425 | 30.155 | 49.084 | 46.969 | 1.00 | 40.99 | C |
| ATOM | 6394 | C | GLY | A | 425 | 29.443 | 47.814 | 46.622 | 1.00 | 41.20 | C |
| ATOM | 6395 | O | GLY | A | 425 | 29.472 | 47.443 | 45.460 | 1.00 | 44.17 | O |
| ATOM | 6396 | N | LEU | A | 426 | 28.805 | 47.141 | 47.566 | 1.00 | 40.36 | N |
| ATOM | 6398 | CA | LEU | A | 426 | 28.240 | 45.842 | 47.258 | 1.00 | 39.92 | C |
| ATOM | 6400 | CB | LEU | A | 426 | 28.082 | 45.026 | 48.527 | 1.00 | 37.76 | C |
| ATOM | 6403 | CG | LEU | A | 426 | 29.426 | 44.744 | 49.170 | 1.00 | 36.86 | C |
| ATOM | 6405 | CD1 | LEU | A | 426 | 29.176 | 43.892 | 50.403 | 1.00 | 38.68 | C |
| ATOM | 6409 | CD2 | LEU | A | 426 | 30.381 | 44.023 | 48.252 | 1.00 | 35.23 | C |
| ATOM | 6413 | C | LEU | A | 426 | 26.932 | 46.001 | 46.557 | 1.00 | 42.18 | C |
| ATOM | 6414 | O | LEU | A | 426 | 25.894 | 45.526 | 47.046 | 1.00 | 44.29 | O |
| ATOM | 6415 | N | ARG | A | 427 | 26.948 | 46.648 | 45.405 | 1.00 | 43.67 | N |
| ATOM | 6417 | CA | ARG | A | 427 | 25.710 | 47.033 | 44.753 | 1.00 | 46.27 | C |
| ATOM | 6419 | CB | ARG | A | 427 | 25.952 | 48.098 | 43.700 | 1.00 | 48.95 | C |
| ATOM | 6422 | CG | ARG | A | 427 | 26.781 | 47.687 | 42.500 | 1.00 | 51.08 | C |
| ATOM | 6425 | CD | ARG | A | 427 | 26.942 | 48.830 | 41.462 | 1.00 | 53.68 | C |
| ATOM | 6428 | NE | ARG | A | 427 | 27.600 | 49.930 | 42.126 | 1.00 | 52.90 | N |
| ATOM | 6430 | CZ | ARG | A | 427 | 28.907 | 49.974 | 42.333 | 1.00 | 54.16 | C |
| ATOM | 6431 | NH1 | ARG | A | 427 | 29.687 | 49.026 | 41.855 | 1.00 | 56.67 | N |
| ATOM | 6434 | NH2 | ARG | A | 427 | 29.469 | 50.978 | 42.973 | 1.00 | 53.42 | N |
| ATOM | 6437 | C | ARG | A | 427 | 24.921 | 45.923 | 44.137 | 1.00 | 46.88 | C |
| ATOM | 6438 | O | ARG | A | 427 | 23.864 | 46.167 | 43.683 | 1.00 | 49.33 | O |
| ATOM | 6439 | N | SER | A | 428 | 25.423 | 44.709 | 44.079 | 1.00 | 45.62 | N |
| ATOM | 6441 | CA | SER | A | 428 | 24.656 | 43.644 | 43.465 | 1.00 | 46.68 | C |
| ATOM | 6443 | CB | SER | A | 428 | 25.514 | 42.826 | 42.506 | 1.00 | 45.99 | C |
| ATOM | 6446 | OG | SER | A | 428 | 25.834 | 43.593 | 41.362 | 1.00 | 47.96 | O |
| ATOM | 6448 | C | SER | A | 428 | 24.148 | 42.720 | 44.486 | 1.00 | 45.95 | C |
| ATOM | 6449 | O | SER | A | 428 | 23.366 | 41.806 | 44.178 | 1.00 | 48.53 | O |
| ATOM | 6450 | N | LEU | A | 429 | 24.620 | 42.903 | 45.703 | 1.00 | 44.50 | N |
| ATOM | 6452 | CA | LEU | A | 429 | 24.329 | 41.963 | 46.753 | 1.00 | 43.25 | C |
| ATOM | 6454 | CB | LEU | A | 429 | 25.197 | 42.215 | 47.923 | 1.00 | 40.85 | C |
| ATOM | 6457 | CG | LEU | A | 429 | 25.039 | 41.129 | 48.966 | 1.00 | 40.60 | C |
| ATOM | 6459 | CD1 | LEU | A | 429 | 25.400 | 39.734 | 48.410 | 1.00 | 40.92 | C |
| ATOM | 6463 | CD2 | LEU | A | 429 | 25.895 | 41.522 | 50.121 | 1.00 | 38.94 | C |
| ATOM | 6467 | C | LEU | A | 429 | 22.889 | 42.116 | 47.185 | 1.00 | 45.57 | C |
| ATOM | 6468 | O | LEU | A | 429 | 22.516 | 43.074 | 47.852 | 1.00 | 46.09 | O |
| ATOM | 6469 | N | LYS | A | 430 | 22.079 | 41.148 | 46.801 | 1.00 | 46.89 | N |
| ATOM | 6471 | CA | LYS | A | 430 | 20.729 | 41.149 | 47.203 | 1.00 | 49.09 | C |
| ATOM | 6473 | CB | LYS | A | 430 | 19.848 | 40.867 | 46.020 | 1.00 | 52.17 | C |
| ATOM | 6476 | CG | LYS | A | 430 | 20.075 | 41.767 | 44.775 | 1.00 | 56.42 | C |
| ATOM | 6479 | CD | LYS | A | 430 | 20.040 | 43.323 | 45.059 | 1.00 | 58.97 | C |
| ATOM | 6482 | CE | LYS | A | 430 | 19.311 | 44.138 | 43.898 | 1.00 | 64.18 | C |
| ATOM | 6485 | NZ | LYS | A | 430 | 18.517 | 45.353 | 44.422 | 1.00 | 67.30 | N |
| ATOM | 6489 | C | LYS | A | 430 | 20.482 | 40.120 | 48.287 | 1.00 | 48.70 | C |
| ATOM | 6490 | O | LYS | A | 430 | 19.396 | 40.095 | 48.817 | 1.00 | 52.84 | O |
| ATOM | 6491 | N | GLU | A | 431 | 21.402 | 39.237 | 48.636 | 1.00 | 45.66 | N |
| ATOM | 6493 | CA | GLU | A | 431 | 20.996 | 38.242 | 49.591 | 1.00 | 44.87 | C |
| ATOM | 6495 | CB | GLU | A | 431 | 20.319 | 37.086 | 48.889 | 1.00 | 46.51 | C |
| ATOM | 6498 | CG | GLU | A | 431 | 20.122 | 35.923 | 49.821 | 1.00 | 47.21 | C |
| ATOM | 6501 | CD | GLU | A | 431 | 19.206 | 34.813 | 49.314 | 1.00 | 54.73 | C |
| ATOM | 6502 | OE1 | GLU | A | 431 | 18.587 | 34.845 | 48.198 | 1.00 | 57.66 | O |
| ATOM | 6503 | OE2 | GLU | A | 431 | 19.061 | 33.865 | 50.108 | 1.00 | 58.94 | O |
| ATOM | 6504 | C | GLU | A | 431 | 22.172 | 37.745 | 50.312 | 1.00 | 42.74 | C |
| ATOM | 6505 | O | GLU | A | 431 | 23.201 | 37.454 | 49.714 | 1.00 | 40.52 | O |
| ATOM | 6506 | N | ILE | A | 432 | 22.020 | 37.676 | 51.635 | 1.00 | 43.15 | N |
| ATOM | 6508 | CA | ILE | A | 432 | 22.953 | 36.946 | 52.541 | 1.00 | 40.67 | C |
| ATOM | 6510 | CB | ILE | A | 432 | 23.656 | 37.901 | 53.551 | 1.00 | 38.41 | C |
| ATOM | 6512 | CG1 | ILE | A | 432 | 24.459 | 38.988 | 52.813 | 1.00 | 36.41 | C |
| ATOM | 6515 | CD1 | ILE | A | 432 | 24.835 | 40.134 | 53.667 | 1.00 | 34.20 | C |
| ATOM | 6519 | CG2 | ILE | A | 432 | 24.583 | 37.096 | 54.405 | 1.00 | 37.20 | C |
| ATOM | 6523 | C | ILE | A | 432 | 22.159 | 35.884 | 53.298 | 1.00 | 42.12 | C |
| ATOM | 6524 | O | ILE | A | 432 | 21.589 | 36.182 | 54.330 | 1.00 | 43.01 | O |
| ATOM | 6525 | N | SER | A | 433 | 22.116 | 34.665 | 52.767 | 1.00 | 43.12 | N |
| ATOM | 6527 | CA | SER | A | 433 | 21.267 | 33.580 | 53.285 | 1.00 | 45.85 | C |
| ATOM | 6529 | CB | SER | A | 433 | 21.474 | 32.275 | 52.493 | 1.00 | 46.97 | C |
| ATOM | 6532 | OG | SER | A | 433 | 21.513 | 32.412 | 51.072 | 1.00 | 47.67 | O |
| ATOM | 6534 | C | SER | A | 433 | 21.414 | 33.251 | 54.795 | 1.00 | 45.97 | C |
| ATOM | 6535 | O | SER | A | 433 | 20.426 | 33.093 | 55.543 | 1.00 | 49.16 | O |
| ATOM | 6536 | N | ASP | A | 434 | 22.636 | 33.142 | 55.246 | 1.00 | 43.68 | N |
| ATOM | 6538 | CA | ASP | A | 434 | 22.897 | 32.829 | 56.616 | 1.00 | 44.35 | C |
| ATOM | 6540 | CB | ASP | A | 434 | 23.092 | 31.311 | 56.710 | 1.00 | 46.18 | C |
| ATOM | 6543 | CG | ASP | A | 434 | 22.775 | 30.720 | 58.098 | 1.00 | 49.52 | C |
| ATOM | 6544 | OD1 | ASP | A | 434 | 22.139 | 31.350 | 58.999 | 1.00 | 50.92 | O |
| ATOM | 6545 | OD2 | ASP | A | 434 | 23.167 | 29.561 | 58.374 | 1.00 | 53.17 | O |
| ATOM | 6546 | C | ASP | A | 434 | 24.121 | 33.598 | 57.103 | 1.00 | 41.73 | C |
| ATOM | 6547 | O | ASP | A | 434 | 24.857 | 34.175 | 56.338 | 1.00 | 39.17 | O |
| ATOM | 6548 | N | GLY | A | 435 | 24.301 | 33.626 | 58.413 | 1.00 | 43.10 | N |
| ATOM | 6550 | CA | GLY | A | 435 | 25.468 | 34.230 | 59.044 | 1.00 | 41.52 | C |
| ATOM | 6553 | C | GLY | A | 435 | 25.300 | 35.685 | 59.493 | 1.00 | 40.59 | C |
| ATOM | 6554 | O | GLY | A | 435 | 24.326 | 36.283 | 59.093 | 1.00 | 42.87 | O |
| ATOM | 6555 | N | ASP | A | 436 | 26.244 | 36.248 | 60.256 | 1.00 | 38.15 | N |
| ATOM | 6557 | CA | ASP | A | 436 | 26.162 | 37.608 | 60.633 | 1.00 | 37.69 | C |
| ATOM | 6559 | CB | ASP | A | 436 | 26.451 | 37.759 | 62.063 | 1.00 | 38.84 | C |
| ATOM | 6562 | CG | ASP | A | 436 | 25.542 | 36.897 | 62.949 | 1.00 | 45.93 | C |
| ATOM | 6563 | OD1 | ASP | A | 436 | 24.475 | 36.346 | 62.465 | 1.00 | 50.44 | O |
| ATOM | 6564 | OD2 | ASP | A | 436 | 25.849 | 36.744 | 64.188 | 1.00 | 48.71 | O |
| ATOM | 6565 | C | ASP | A | 436 | 27.101 | 38.463 | 59.884 | 1.00 | 35.36 | C |
| ATOM | 6566 | O | ASP | A | 436 | 28.171 | 38.045 | 59.505 | 1.00 | 34.46 | O |
| ATOM | 6567 | N | VAL | A | 437 | 26.675 | 39.699 | 59.666 | 1.00 | 34.91 | N |
| ATOM | 6569 | CA | VAL | A | 437 | 27.539 | 40.698 | 59.138 | 1.00 | 32.89 | C |
| ATOM | 6571 | CB | VAL | A | 437 | 26.807 | 41.670 | 58.331 | 1.00 | 33.15 | C |
| ATOM | 6573 | CG1 | VAL | A | 437 | 27.752 | 42.703 | 57.809 | 1.00 | 33.08 | C |
| ATOM | 6577 | CG2 | VAL | A | 437 | 26.255 | 40.973 | 57.202 | 1.00 | 34.45 | C |
| ATOM | 6581 | C | VAL | A | 437 | 28.177 | 41.428 | 60.248 | 1.00 | 32.40 | C |
| ATOM | 6582 | O | VAL | A | 437 | 27.550 | 41.789 | 61.196 | 1.00 | 33.85 | O |
| ATOM | 6583 | N | ILE | A | 438 | 29.465 | 41.596 | 60.128 | 1.00 | 31.56 | N |
| ATOM | 6585 | CA | ILE | A | 438 | 30.196 | 42.496 | 60.980 | 1.00 | 32.05 | C |
| ATOM | 6587 | CB | ILE | A | 438 | 31.067 | 41.725 | 62.017 | 1.00 | 31.78 | C |
| ATOM | 6589 | CG1 | ILE | A | 438 | 31.917 | 42.722 | 62.782 | 1.00 | 33.81 | C |
| ATOM | 6592 | CD1 | ILE | A | 438 | 31.980 | 42.501 | 64.214 | 1.00 | 34.56 | C |
| ATOM | 6596 | CG2 | ILE | A | 438 | 31.869 | 40.711 | 61.404 | 1.00 | 28.81 | C |
| ATOM | 6600 | C | ILE | A | 438 | 31.049 | 43.403 | 60.138 | 1.00 | 30.73 | C |
| ATOM | 6601 | O | ILE | A | 438 | 31.771 | 42.980 | 59.286 | 1.00 | 30.46 | O |
| ATOM | 6602 | N | ILE | A | 439 | 30.969 | 44.668 | 60.434 | 1.00 | 31.67 | N |
| ATOM | 6604 | CA | ILE | A | 439 | 31.661 | 45.697 | 59.693 | 1.00 | 31.64 | C |
| ATOM | 6606 | CB | ILE | A | 439 | 30.627 | 46.403 | 58.860 | 1.00 | 33.32 | C |
| ATOM | 6608 | CG1 | ILE | A | 439 | 30.097 | 45.368 | 57.903 | 1.00 | 33.68 | C |
| ATOM | 6611 | CD1 | ILE | A | 439 | 29.445 | 45.900 | 56.843 | 1.00 | 37.03 | C |
| ATOM | 6615 | CG2 | ILE | A | 439 | 31.173 | 47.672 | 58.251 | 1.00 | 32.18 | C |
| ATOM | 6619 | C | ILE | A | 439 | 32.188 | 46.642 | 60.672 | 1.00 | 32.06 | C |
| ATOM | 6620 | O | ILE | A | 439 | 31.404 | 47.452 | 61.174 | 1.00 | 33.52 | O |
| ATOM | 6621 | N | SER | A | 440 | 33.472 | 46.512 | 60.990 | 1.00 | 31.48 | N |
| ATOM | 6623 | CA | SER | A | 440 | 34.064 | 47.352 | 61.993 | 1.00 | 32.75 | C |
| ATOM | 6625 | CB | SER | A | 440 | 34.033 | 46.654 | 63.346 | 1.00 | 34.08 | C |
| ATOM | 6628 | OG | SER | A | 440 | 35.219 | 46.099 | 63.813 | 1.00 | 30.62 | O |
| ATOM | 6630 | C | SER | A | 440 | 35.423 | 47.965 | 61.710 | 1.00 | 33.20 | C |
| ATOM | 6631 | O | SER | A | 440 | 36.301 | 47.431 | 61.050 | 1.00 | 31.65 | O |
| ATOM | 6632 | N | GLY | A | 441 | 35.564 | 49.154 | 62.243 | 1.00 | 34.51 | N |
| ATOM | 6634 | CA | GLY | A | 441 | 36.833 | 49.803 | 62.243 | 1.00 | 35.00 | C |
| ATOM | 6637 | C | GLY | A | 441 | 37.257 | 50.356 | 60.936 | 1.00 | 34.41 | C |
| ATOM | 6638 | O | GLY | A | 441 | 38.416 | 50.475 | 60.699 | 1.00 | 36.85 | O |
| ATOM | 6639 | N | ASN | A | 442 | 36.327 | 50.764 | 60.121 | 1.00 | 34.47 | N |
| ATOM | 6641 | CA | ASN | A | 442 | 36.633 | 51.433 | 58.902 | 1.00 | 34.18 | C |
| ATOM | 6643 | CB | ASN | A | 442 | 35.722 | 50.813 | 57.889 | 1.00 | 32.80 | C |
| ATOM | 6646 | CG | ASN | A | 442 | 35.795 | 49.277 | 57.918 | 1.00 | 31.45 | C |
| ATOM | 6647 | OD1 | ASN | A | 442 | 36.767 | 48.706 | 57.513 | 1.00 | 31.51 | O |
| ATOM | 6648 | ND2 | ASN | A | 442 | 34.759 | 48.620 | 58.385 | 1.00 | 33.37 | N |
| ATOM | 6651 | C | ASN | A | 442 | 36.483 | 52.975 | 59.097 | 1.00 | 36.49 | C |
| ATOM | 6652 | O | ASN | A | 442 | 35.399 | 53.472 | 58.871 | 1.00 | 37.28 | O |
| ATOM | 6653 | N | LYS | A | 443 | 37.537 | 53.694 | 59.570 | 1.00 | 37.27 | N |
| ATOM | 6655 | CA | LYS | A | 443 | 37.443 | 55.111 | 59.920 | 1.00 | 40.56 | C |
| ATOM | 6657 | CB | LYS | A | 443 | 38.759 | 55.895 | 59.850 | 1.00 | 42.83 | C |
| ATOM | 6660 | CG | LYS | A | 443 | 39.988 | 55.442 | 60.495 | 1.00 | 45.94 | C |
| ATOM | 6663 | CD | LYS | A | 443 | 41.237 | 56.329 | 60.166 | 1.00 | 47.16 | C |
| ATOM | 6666 | CE | LYS | A | 443 | 42.546 | 55.495 | 60.405 | 1.00 | 49.15 | C |
| ATOM | 6669 | NZ | LYS | A | 443 | 43.707 | 55.709 | 59.389 | 1.00 | 50.56 | N |
| ATOM | 6673 | C | LYS | A | 443 | 36.582 | 55.947 | 58.945 | 1.00 | 42.61 | C |
| ATOM | 6674 | O | LYS | A | 443 | 35.994 | 56.946 | 59.344 | 1.00 | 44.63 | O |
| ATOM | 6675 | N | ASN | A | 444 | 36.652 | 55.608 | 57.658 | 1.00 | 42.59 | N |
| ATOM | 6677 | CA | ASN | A | 444 | 36.171 | 56.418 | 56.545 | 1.00 | 44.01 | C |
| ATOM | 6679 | CB | ASN | A | 444 | 37.294 | 56.464 | 55.497 | 1.00 | 44.13 | C |
| ATOM | 6682 | CG | ASN | A | 444 | 38.405 | 57.361 | 55.910 | 1.00 | 46.10 | C |
| ATOM | 6683 | OD1 | ASN | A | 444 | 38.183 | 58.301 | 56.639 | 1.00 | 50.68 | O |
| ATOM | 6684 | ND2 | ASN | A | 444 | 39.594 | 57.100 | 55.461 | 1.00 | 47.38 | N |
| ATOM | 6687 | C | ASN | A | 444 | 34.921 | 55.847 | 55.902 | 1.00 | 43.68 | C |
| ATOM | 6688 | O | ASN | A | 444 | 34.542 | 56.270 | 54.825 | 1.00 | 45.84 | O |
| ATOM | 6689 | N | LEU | A | 445 | 34.283 | 54.866 | 56.539 | 1.00 | 42.24 | N |
| ATOM | 6691 | CA | LEU | A | 445 | 33.301 | 54.082 | 55.849 | 1.00 | 41.55 | C |
| ATOM | 6693 | CB | LEU | A | 445 | 33.208 | 52.701 | 56.401 | 1.00 | 38.97 | C |
| ATOM | 6696 | CG | LEU | A | 445 | 32.117 | 51.924 | 55.680 | 1.00 | 39.43 | C |
| ATOM | 6698 | CD1 | LEU | A | 445 | 32.454 | 51.904 | 54.221 | 1.00 | 41.08 | C |
| ATOM | 6702 | CD2 | LEU | A | 445 | 31.954 | 50.485 | 56.169 | 1.00 | 37.35 | C |
| ATOM | 6706 | C | LEU | A | 445 | 31.995 | 54.746 | 56.056 | 1.00 | 44.65 | C |
| ATOM | 6707 | O | LEU | A | 445 | 31.635 | 55.160 | 57.193 | 1.00 | 46.34 | O |
| ATOM | 6708 | N | CYS | A | 446 | 31.267 | 54.879 | 54.966 | 1.00 | 45.65 | N |
| ATOM | 6710 | CA | CYS | A | 446 | 29.956 | 55.494 | 55.049 | 1.00 | 47.96 | C |
| ATOM | 6712 | CB | CYS | A | 446 | 29.980 | 56.842 | 54.346 | 1.00 | 51.65 | C |
| ATOM | 6715 | SG | CYS | A | 446 | 30.690 | 58.223 | 55.323 | 1.00 | 56.94 | S |
| ATOM | 6716 | C | CYS | A | 446 | 28.930 | 54.580 | 54.454 | 1.00 | 45.78 | C |
| ATOM | 6717 | O | CYS | A | 446 | 29.247 | 53.516 | 53.948 | 1.00 | 45.34 | O |
| ATOM | 6718 | N | TYR | A | 447 | 27.698 | 54.972 | 54.622 | 1.00 | 46.60 | N |
| ATOM | 6720 | CA | TYR | A | 447 | 26.536 | 54.438 | 53.926 | 1.00 | 46.93 | C |
| ATOM | 6722 | CB | TYR | A | 447 | 26.727 | 54.312 | 52.366 | 1.00 | 46.78 | C |
| ATOM | 6725 | CG | TYR | A | 447 | 27.370 | 55.536 | 51.702 | 1.00 | 48.48 | C |
| ATOM | 6726 | CD1 | TYR | A | 447 | 28.763 | 55.661 | 51.604 | 1.00 | 48.29 | C |
| ATOM | 6728 | CE1 | TYR | A | 447 | 29.359 | 56.796 | 51.020 | 1.00 | 49.65 | C |
| ATOM | 6730 | CZ | TYR | A | 447 | 28.561 | 57.823 | 50.584 | 1.00 | 53.33 | C |
| ATOM | 6731 | OH | TYR | A | 447 | 29.156 | 58.934 | 50.022 | 1.00 | 56.47 | O |
| ATOM | 6733 | CE2 | TYR | A | 447 | 27.188 | 57.718 | 50.684 | 1.00 | 52.55 | C |
| ATOM | 6735 | CD2 | TYR | A | 447 | 26.603 | 56.594 | 51.237 | 1.00 | 50.12 | C |
| ATOM | 6737 | C | TYR | A | 447 | 26.006 | 53.182 | 54.579 | 1.00 | 45.12 | C |
| ATOM | 6738 | O | TYR | A | 447 | 24.828 | 52.906 | 54.450 | 1.00 | 46.05 | O |
| ATOM | 6739 | N | ALA | A | 448 | 26.825 | 52.441 | 55.307 | 1.00 | 42.46 | N |
| ATOM | 6741 | CA | ALA | A | 448 | 26.412 | 51.097 | 55.672 | 1.00 | 42.12 | C |
| ATOM | 6743 | CB | ALA | A | 448 | 27.486 | 50.466 | 56.386 | 1.00 | 40.08 | C |
| ATOM | 6747 | C | ALA | A | 448 | 25.164 | 51.065 | 56.550 | 1.00 | 45.51 | C |
| ATOM | 6748 | O | ALA | A | 448 | 24.377 | 50.118 | 56.546 | 1.00 | 46.84 | O |
| ATOM | 6749 | N | ASN | A | 449 | 25.042 | 52.116 | 57.349 | 1.00 | 48.30 | N |
| ATOM | 6751 | CA | ASN | A | 449 | 24.032 | 52.298 | 58.385 | 1.00 | 50.40 | C |
| ATOM | 6753 | CB | ASN | A | 449 | 24.550 | 53.507 | 59.267 | 1.00 | 51.87 | C |
| ATOM | 6756 | CG | ASN | A | 449 | 24.734 | 54.895 | 58.395 | 1.00 | 57.64 | C |
| ATOM | 6757 | OD1 | ASN | A | 449 | 25.688 | 55.006 | 57.531 | 1.00 | 53.24 | O |
| ATOM | 6758 | ND2 | ASN | A | 449 | 23.747 | 55.920 | 58.585 | 1.00 | 59.18 | N |
| ATOM | 6761 | C | ASN | A | 449 | 22.672 | 52.509 | 57.647 | 1.00 | 52.26 | C |
| ATOM | 6762 | O | ASN | A | 449 | 21.637 | 52.128 | 58.077 | 1.00 | 53.29 | O |
| ATOM | 6763 | N | THR | A | 450 | 22.728 | 53.091 | 56.469 | 1.00 | 53.13 | N |
| ATOM | 6765 | CA | THR | A | 450 | 21.597 | 53.268 | 55.534 | 1.00 | 54.99 | C |
| ATOM | 6767 | CB | THR | A | 450 | 22.266 | 53.742 | 54.252 | 1.00 | 54.62 | C |
| ATOM | 6769 | OG1 | THR | A | 450 | 22.367 | 55.152 | 54.295 | 1.00 | 55.61 | O |
| ATOM | 6771 | CG2 | THR | A | 450 | 21.443 | 53.564 | 53.124 | 1.00 | 57.50 | C |
| ATOM | 6775 | C | THR | A | 450 | 20.713 | 52.104 | 55.065 | 1.00 | 55.13 | C |
| ATOM | 6776 | O | THR | A | 450 | 19.534 | 52.252 | 54.725 | 1.00 | 57.64 | O |
| ATOM | 6777 | N | ILE | A | 451 | 21.340 | 50.971 | 54.847 | 1.00 | 53.09 | N |
| ATOM | 6779 | CA | ILE | A | 451 | 20.639 | 49.838 | 54.231 | 1.00 | 52.81 | C |
| ATOM | 6781 | CB | ILE | A | 451 | 21.672 | 48.959 | 53.650 | 1.00 | 48.99 | C |
| ATOM | 6783 | CG1 | ILE | A | 451 | 21.964 | 49.458 | 52.272 | 1.00 | 48.98 | C |
| ATOM | 6786 | CD1 | ILE | A | 451 | 23.287 | 48.919 | 51.907 | 1.00 | 51.48 | C |
| ATOM | 6790 | CG2 | ILE | A | 451 | 21.238 | 47.512 | 53.660 | 1.00 | 48.80 | C |
| ATOM | 6794 | C | ILE | A | 451 | 19.855 | 49.041 | 55.251 | 1.00 | 53.28 | C |
| ATOM | 6795 | O | ILE | A | 451 | 20.291 | 48.882 | 56.403 | 1.00 | 53.75 | O |
| ATOM | 6796 | N | ASN | A | 452 | 18.729 | 48.501 | 54.859 | 1.00 | 54.61 | N |
| ATOM | 6798 | CA | ASN | A | 452 | 18.075 | 47.564 | 55.753 | 1.00 | 54.85 | C |
| ATOM | 6800 | CB | ASN | A | 452 | 16.582 | 47.583 | 55.595 | 1.00 | 58.78 | C |
| ATOM | 6803 | CG | ASN | A | 452 | 15.898 | 46.688 | 56.573 | 1.00 | 60.06 | C |
| ATOM | 6804 | OD1 | ASN | A | 452 | 16.477 | 46.247 | 57.535 | 1.00 | 60.16 | O |
| ATOM | 6805 | ND2 | ASN | A | 452 | 14.630 | 46.447 | 56.348 | 1.00 | 66.13 | N |
| ATOM | 6808 | C | ASN | A | 452 | 18.584 | 46.161 | 55.514 | 1.00 | 51.88 | C |
| ATOM | 6809 | O | ASN | A | 452 | 18.079 | 45.429 | 54.688 | 1.00 | 51.93 | O |
| ATOM | 6810 | N | TRP | A | 453 | 19.574 | 45.788 | 56.289 | 1.00 | 49.02 | N |
| ATOM | 6812 | CA | TRP | A | 453 | 20.200 | 44.509 | 56.111 | 1.00 | 47.00 | C |
| ATOM | 6814 | CB | TRP | A | 453 | 21.380 | 44.370 | 57.080 | 1.00 | 43.94 | C |
| ATOM | 6817 | CG | TRP | A | 453 | 22.510 | 45.291 | 56.788 | 1.00 | 42.79 | C |
| ATOM | 6818 | CD1 | TRP | A | 453 | 22.646 | 46.594 | 57.203 | 1.00 | 43.19 | C |
| ATOM | 6820 | NE1 | TRP | A | 453 | 23.832 | 47.126 | 56.749 | 1.00 | 42.40 | N |
| ATOM | 6822 | CE2 | TRP | A | 453 | 24.515 | 46.164 | 56.055 | 1.00 | 41.54 | C |
| ATOM | 6823 | CD2 | TRP | A | 453 | 23.706 | 44.986 | 56.055 | 1.00 | 40.84 | C |
| ATOM | 6824 | CE3 | TRP | A | 453 | 24.181 | 43.846 | 55.397 | 1.00 | 37.49 | C |
| ATOM | 6826 | CZ3 | TRP | A | 453 | 25.422 | 43.910 | 54.728 | 1.00 | 36.87 | C |
| ATOM | 6828 | CH2 | TRP | A | 453 | 26.216 | 45.085 | 54.767 | 1.00 | 37.26 | C |
| ATOM | 6830 | CZ2 | TRP | A | 453 | 25.771 | 46.226 | 55.419 | 1.00 | 40.29 | C |
| ATOM | 6832 | C | TRP | A | 453 | 19.210 | 43.347 | 56.304 | 1.00 | 48.34 | C |
| ATOM | 6833 | O | TRP | A | 453 | 19.523 | 42.225 | 55.960 | 1.00 | 48.61 | O |
| ATOM | 6834 | N | ALA | A | 454 | 18.049 | 43.569 | 56.872 | 1.00 | 50.12 | N |
| ATOM | 6836 | CA | ALA | A | 454 | 17.163 | 42.440 | 57.046 | 1.00 | 52.09 | C |
| ATOM | 6838 | CB | ALA | A | 454 | 16.100 | 42.742 | 58.175 | 1.00 | 55.94 | C |
| ATOM | 6842 | C | ALA | A | 454 | 16.497 | 41.955 | 55.731 | 1.00 | 52.76 | C |
| ATOM | 6843 | O | ALA | A | 454 | 16.033 | 40.830 | 55.669 | 1.00 | 53.28 | O |
| ATOM | 6844 | N | ALA | A | 455 | 16.438 | 42.796 | 54.699 | 1.00 | 53.04 | N |
| ATOM | 6846 | CA | ALA | A | 455 | 15.958 | 42.371 | 53.388 | 1.00 | 54.61 | C |
| ATOM | 6848 | CB | ALA | A | 455 | 15.611 | 43.600 | 52.415 | 1.00 | 55.56 | C |
| ATOM | 6852 | C | ALA | A | 455 | 17.019 | 41.447 | 52.780 | 1.00 | 52.15 | C |
| ATOM | 6853 | O | ALA | A | 455 | 16.713 | 40.564 | 51.986 | 1.00 | 53.84 | O |
| ATOM | 6854 | N | LEU | A | 456 | 18.278 | 41.584 | 53.144 | 1.00 | 48.88 | N |
| ATOM | 6856 | CA | LEU | A | 456 | 19.207 | 40.624 | 52.575 | 1.00 | 47.66 | C |
| ATOM | 6858 | CB | LEU | A | 456 | 20.629 | 41.111 | 52.554 | 1.00 | 44.76 | C |
| ATOM | 6861 | CG | LEU | A | 456 | 20.822 | 42.604 | 52.299 | 1.00 | 47.13 | C |
| ATOM | 6863 | CD1 | LEU | A | 456 | 22.273 | 42.896 | 52.676 | 1.00 | 47.80 | C |
| ATOM | 6867 | CD2 | LEU | A | 456 | 20.538 | 43.052 | 50.860 | 1.00 | 46.95 | C |
| ATOM | 6871 | C | LEU | A | 456 | 19.131 | 39.306 | 53.285 | 1.00 | 47.43 | C |
| ATOM | 6872 | O | LEU | A | 456 | 19.433 | 38.295 | 52.722 | 1.00 | 48.81 | O |
| ATOM | 6873 | N | PHE | A | 457 | 18.713 | 39.299 | 54.520 | 1.00 | 48.14 | N |
| ATOM | 6875 | CA | PHE | A | 457 | 18.752 | 38.096 | 55.293 | 1.00 | 48.22 | C |
| ATOM | 6877 | CB | PHE | A | 457 | 18.711 | 38.449 | 56.766 | 1.00 | 48.21 | C |
| ATOM | 6880 | CG | PHE | A | 457 | 19.963 | 39.062 | 57.277 | 1.00 | 44.36 | C |
| ATOM | 6881 | CD1 | PHE | A | 457 | 19.934 | 39.820 | 58.425 | 1.00 | 44.86 | C |
| ATOM | 6883 | CE1 | PHE | A | 457 | 21.026 | 40.346 | 58.984 | 1.00 | 42.67 | C |
| ATOM | 6885 | CZ | PHE | A | 457 | 22.226 | 40.151 | 58.400 | 1.00 | 43.38 | C |
| ATOM | 6887 | CE2 | PHE | A | 457 | 22.294 | 39.405 | 57.247 | 1.00 | 43.86 | C |
| ATOM | 6889 | CD2 | PHE | A | 457 | 21.148 | 38.843 | 56.690 | 1.00 | 42.41 | C |
| ATOM | 6891 | C | PHE | A | 457 | 17.647 | 37.119 | 54.932 | 1.00 | 51.40 | C |
| ATOM | 6892 | O | PHE | A | 457 | 16.662 | 37.500 | 54.357 | 1.00 | 52.54 | O |
| ATOM | 6893 | N | GLY | A | 458 | 17.898 | 35.854 | 55.274 | 1.00 | 52.35 | N |
| ATOM | 6895 | CA | GLY | A | 458 | 17.023 | 34.744 | 55.030 | 1.00 | 56.05 | C |
| ATOM | 6898 | C | GLY | A | 458 | 16.661 | 33.924 | 56.238 | 1.00 | 58.52 | C |
| ATOM | 6899 | O | GLY | A | 458 | 15.591 | 33.315 | 56.320 | 1.00 | 63.06 | O |
| ATOM | 6900 | N | THR | A | 459 | 17.597 | 33.823 | 57.145 | 1.00 | 57.90 | N |
| ATOM | 6902 | CA | THR | A | 459 | 17.361 | 33.198 | 58.447 | 1.00 | 60.11 | C |
| ATOM | 6904 | CB | THR | A | 459 | 18.698 | 32.524 | 59.028 | 1.00 | 58.33 | C |
| ATOM | 6906 | OG1 | THR | A | 459 | 19.443 | 31.758 | 58.027 | 1.00 | 56.59 | O |
| ATOM | 6908 | CG2 | THR | A | 459 | 18.397 | 31.515 | 60.156 | 1.00 | 61.86 | C |
| ATOM | 6912 | C | THR | A | 459 | 16.973 | 34.339 | 59.362 | 1.00 | 60.43 | C |
| ATOM | 6913 | O | THR | A | 459 | 17.504 | 35.455 | 59.293 | 1.00 | 58.76 | O |
| ATOM | 6914 | N | SER | A | 460 | 16.055 | 34.063 | 60.248 | 1.00 | 63.77 | N |
| ATOM | 6916 | CA | SER | A | 460 | 15.832 | 34.971 | 61.348 | 1.00 | 64.47 | C |
| ATOM | 6918 | CB | SER | A | 460 | 14.427 | 34.808 | 61.931 | 1.00 | 69.15 | C |
| ATOM | 6921 | OG | SER | A | 460 | 14.550 | 34.108 | 63.143 | 1.00 | 71.63 | O |
| ATOM | 6923 | C | SER | A | 460 | 16.934 | 34.647 | 62.384 | 1.00 | 62.22 | C |
| ATOM | 6924 | O | SER | A | 460 | 17.590 | 33.555 | 62.356 | 1.00 | 61.49 | O |
| ATOM | 6925 | N | GLY | A | 461 | 17.164 | 35.602 | 63.277 | 1.00 | 60.59 | N |
| ATOM | 6927 | CA | GLY | A | 461 | 18.316 | 35.494 | 64.123 | 1.00 | 57.63 | C |
| ATOM | 6930 | C | GLY | A | 461 | 19.585 | 35.989 | 63.511 | 1.00 | 52.41 | C |
| ATOM | 6931 | O | GLY | A | 461 | 20.446 | 36.242 | 64.286 | 1.00 | 52.50 | O |
| ATOM | 6932 | N | GLN | A | 462 | 19.741 | 36.137 | 62.195 | 1.00 | 49.52 | N |
| ATOM | 6934 | CA | GLN | A | 462 | 20.925 | 36.822 | 61.668 | 1.00 | 45.64 | C |
| ATOM | 6936 | CB | GLN | A | 462 | 20.952 | 36.897 | 60.163 | 1.00 | 43.36 | C |
| ATOM | 6939 | CG | GLN | A | 462 | 21.317 | 35.605 | 59.465 | 1.00 | 43.36 | C |
| ATOM | 6942 | CD | GLN | A | 462 | 21.144 | 35.662 | 57.939 | 1.00 | 42.21 | C |
| ATOM | 6943 | OE1 | GLN | A | 462 | 20.022 | 35.477 | 57.420 | 1.00 | 43.94 | O |
| ATOM | 6944 | NE2 | GLN | A | 462 | 22.221 | 35.915 | 57.237 | 1.00 | 38.31 | N |
| ATOM | 6947 | C | GLN | A | 462 | 21.059 | 38.245 | 62.227 | 1.00 | 46.14 | C |
| ATOM | 6948 | O | GLN | A | 462 | 20.063 | 38.974 | 62.410 | 1.00 | 47.92 | O |
| ATOM | 6949 | N | LYS | A | 463 | 22.288 | 38.625 | 62.523 | 1.00 | 44.23 | N |
| ATOM | 6951 | CA | LYS | A | 463 | 22.533 | 39.895 | 63.147 | 1.00 | 45.46 | C |
| ATOM | 6953 | CB | LYS | A | 463 | 23.017 | 39.668 | 64.596 | 1.00 | 46.79 | C |
| ATOM | 6956 | CG | LYS | A | 463 | 21.984 | 38.954 | 65.430 | 1.00 | 55.05 | C |
| ATOM | 6959 | CD | LYS | A | 463 | 22.139 | 39.020 | 67.026 | 1.00 | 59.62 | C |
| ATOM | 6962 | CE | LYS | A | 463 | 20.716 | 38.916 | 67.717 | 1.00 | 64.36 | C |
| ATOM | 6965 | NZ | LYS | A | 463 | 20.821 | 38.672 | 69.237 | 1.00 | 69.48 | N |
| ATOM | 6969 | C | LYS | A | 463 | 23.603 | 40.691 | 62.389 | 1.00 | 42.96 | C |
| ATOM | 6970 | O | LYS | A | 463 | 24.382 | 40.166 | 61.581 | 1.00 | 39.97 | O |
| ATOM | 6971 | N | THR | A | 464 | 23.730 | 41.932 | 62.818 | 1.00 | 43.35 | N |
| ATOM | 6973 | CA | THR | A | 464 | 24.580 | 42.878 | 62.188 | 1.00 | 42.23 | C |
| ATOM | 6975 | CB | THR | A | 464 | 23.583 | 43.748 | 61.509 | 1.00 | 44.72 | C |
| ATOM | 6977 | OG1 | THR | A | 464 | 23.198 | 43.095 | 60.276 | 1.00 | 46.76 | O |
| ATOM | 6979 | CG2 | THR | A | 464 | 24.182 | 45.089 | 61.116 | 1.00 | 46.18 | C |
| ATOM | 6983 | C | THR | A | 464 | 25.331 | 43.693 | 63.198 | 1.00 | 41.03 | C |
| ATOM | 6984 | O | THR | A | 464 | 24.720 | 44.166 | 64.099 | 1.00 | 43.87 | O |
| ATOM | 6985 | N | LYS | A | 465 | 26.616 | 43.918 | 63.032 | 1.00 | 38.29 | N |
| ATOM | 6987 | CA | LYS | A | 465 | 27.350 | 44.883 | 63.862 | 1.00 | 37.96 | C |
| ATOM | 6989 | CB | LYS | A | 465 | 28.365 | 44.234 | 64.770 | 1.00 | 38.00 | C |
| ATOM | 6992 | CG | LYS | A | 465 | 27.843 | 43.601 | 66.020 | 1.00 | 39.95 | C |
| ATOM | 6995 | CD | LYS | A | 465 | 29.041 | 42.928 | 66.733 | 1.00 | 39.30 | C |
| ATOM | 6998 | CE | LYS | A | 465 | 28.550 | 41.917 | 67.693 | 1.00 | 43.12 | C |
| ATOM | 7001 | NZ | LYS | A | 465 | 29.446 | 41.823 | 68.859 | 1.00 | 47.34 | N |
| ATOM | 7005 | C | LYS | A | 465 | 28.107 | 45.855 | 62.997 | 1.00 | 35.71 | C |
| ATOM | 7006 | O | LYS | A | 465 | 29.066 | 45.520 | 62.377 | 1.00 | 34.33 | O |
| ATOM | 7007 | N | ILE | A | 466 | 27.686 | 47.083 | 63.039 | 1.00 | 36.59 | N |
| ATOM | 7009 | CA | ILE | A | 466 | 28.230 | 48.113 | 62.287 | 1.00 | 36.36 | C |
| ATOM | 7011 | CB | ILE | A | 466 | 27.149 | 48.658 | 61.458 | 1.00 | 37.62 | C |
| ATOM | 7013 | CG1 | ILE | A | 466 | 26.638 | 47.521 | 60.575 | 1.00 | 39.37 | C |
| ATOM | 7016 | CD1 | ILE | A | 466 | 25.425 | 47.855 | 59.830 | 1.00 | 43.42 | C |
| ATOM | 7020 | CG2 | ILE | A | 466 | 27.645 | 49.806 | 60.602 | 1.00 | 36.38 | C |
| ATOM | 7024 | C | ILE | A | 466 | 28.660 | 49.060 | 63.328 | 1.00 | 39.00 | C |
| ATOM | 7025 | O | ILE | A | 466 | 27.884 | 49.879 | 63.803 | 1.00 | 42.79 | O |
| ATOM | 7026 | N | ILE | A | 467 | 29.957 | 49.024 | 63.565 | 1.00 | 39.11 | N |
| ATOM | 7028 | CA | ILE | A | 467 | 30.596 | 49.432 | 64.779 | 1.00 | 40.37 | C |
| ATOM | 7030 | CB | ILE | A | 467 | 31.219 | 48.212 | 65.331 | 1.00 | 40.16 | C |
| ATOM | 7032 | CG1 | ILE | A | 467 | 30.370 | 47.332 | 66.163 | 1.00 | 42.73 | C |
| ATOM | 7035 | CD1 | ILE | A | 467 | 31.377 | 45.982 | 66.241 | 1.00 | 40.48 | C |
| ATOM | 7039 | CG2 | ILE | A | 467 | 32.262 | 48.549 | 66.279 | 1.00 | 45.19 | C |
| ATOM | 7043 | C | ILE | A | 467 | 31.840 | 50.203 | 64.366 | 1.00 | 39.14 | C |
| ATOM | 7044 | O | ILE | A | 467 | 32.618 | 49.668 | 63.547 | 1.00 | 36.89 | O |
| ATOM | 7045 | N | SER | A | 468 | 32.116 | 51.332 | 65.040 | 1.00 | 40.22 | N |
| ATOM | 7047 | CA | SER | A | 468 | 33.370 | 52.094 | 64.911 | 1.00 | 39.83 | C |
| ATOM | 7049 | CB | SER | A | 468 | 34.462 | 51.450 | 65.689 | 1.00 | 40.10 | C |
| ATOM | 7052 | OG | SER | A | 468 | 35.683 | 52.125 | 65.536 | 1.00 | 44.57 | O |
| ATOM | 7054 | C | SER | A | 468 | 33.775 | 52.258 | 63.472 | 1.00 | 38.05 | C |
| ATOM | 7055 | O | SER | A | 468 | 34.825 | 51.854 | 63.028 | 1.00 | 35.32 | O |
| ATOM | 7056 | N | ASN | A | 469 | 32.855 | 52.838 | 62.716 | 1.00 | 39.19 | N |
| ATOM | 7058 | CA | ASN | A | 469 | 33.116 | 53.297 | 61.394 | 1.00 | 38.06 | C |
| ATOM | 7060 | CB | ASN | A | 469 | 32.120 | 52.660 | 60.473 | 1.00 | 37.00 | C |
| ATOM | 7063 | CG | ASN | A | 469 | 32.229 | 51.158 | 60.463 | 1.00 | 32.67 | C |
| ATOM | 7064 | OD1 | ASN | A | 469 | 33.227 | 50.572 | 60.068 | 1.00 | 31.21 | O |
| ATOM | 7065 | ND2 | ASN | A | 469 | 31.183 | 50.536 | 60.930 | 1.00 | 33.32 | N |
| ATOM | 7068 | C | ASN | A | 469 | 33.072 | 54.801 | 61.447 | 1.00 | 40.14 | C |
| ATOM | 7069 | O | ASN | A | 469 | 33.215 | 55.332 | 62.487 | 1.00 | 41.27 | O |
| ATOM | 7070 | N | ARG | A | 470 | 32.989 | 55.479 | 60.332 | 1.00 | 42.08 | N |
| ATOM | 7072 | CA | ARG | A | 470 | 32.665 | 56.891 | 60.330 | 1.00 | 46.67 | C |
| ATOM | 7074 | CB | ARG | A | 470 | 32.810 | 57.409 | 58.920 | 1.00 | 47.34 | C |
| ATOM | 7077 | CG | ARG | A | 470 | 32.742 | 58.880 | 58.850 | 1.00 | 52.09 | C |
| ATOM | 7080 | CD | ARG | A | 470 | 34.050 | 59.464 | 58.571 | 1.00 | 55.79 | C |
| ATOM | 7083 | NE | ARG | A | 470 | 33.944 | 60.651 | 57.785 | 1.00 | 61.59 | N |
| ATOM | 7085 | CZ | ARG | A | 470 | 34.946 | 61.179 | 57.104 | 1.00 | 64.65 | C |
| ATOM | 7086 | NH1 | ARG | A | 470 | 36.169 | 60.602 | 57.129 | 1.00 | 61.27 | N |
| ATOM | 7089 | NH2 | ARG | A | 470 | 34.710 | 62.300 | 56.374 | 1.00 | 67.54 | N |
| ATOM | 7092 | C | ARG | A | 470 | 31.205 | 57.060 | 60.710 | 1.00 | 49.51 | C |
| ATOM | 7093 | O | ARG | A | 470 | 30.334 | 56.385 | 60.116 | 1.00 | 51.46 | O |
| ATOM | 7094 | N | GLY | A | 471 | 30.864 | 57.940 | 61.623 | 1.00 | 52.32 | N |
| ATOM | 7096 | CA | GLY | A | 471 | 29.444 | 58.051 | 61.999 | 1.00 | 54.41 | C |
| ATOM | 7099 | C | GLY | A | 471 | 28.409 | 58.690 | 61.044 | 1.00 | 56.89 | C |
| ATOM | 7100 | O | GLY | A | 471 | 28.771 | 59.570 | 60.204 | 1.00 | 58.19 | O |
| ATOM | 7101 | N | ALA | A | 472 | 27.117 | 58.276 | 61.201 | 1.00 | 57.50 | N |
| ATOM | 7103 | CA | ALA | A | 472 | 25.991 | 58.836 | 60.402 | 1.00 | 59.48 | C |
| ATOM | 7105 | CB | ALA | A | 472 | 24.529 | 58.422 | 60.926 | 1.00 | 59.82 | C |
| ATOM | 7109 | C | ALA | A | 472 | 26.180 | 60.340 | 60.361 | 1.00 | 62.54 | C |
| ATOM | 7110 | O | ALA | A | 472 | 25.935 | 60.936 | 59.337 | 1.00 | 64.71 | O |
| ATOM | 7111 | N | ASN | A | 473 | 26.708 | 60.910 | 61.448 | 1.00 | 63.09 | N |
| ATOM | 7113 | CA | ASN | A | 473 | 26.888 | 62.359 | 61.610 | 1.00 | 66.39 | C |
| ATOM | 7115 | CB | ASN | A | 473 | 27.229 | 62.615 | 63.117 | 1.00 | 67.84 | C |
| ATOM | 7118 | CG | ASN | A | 473 | 27.465 | 64.101 | 63.481 | 1.00 | 71.47 | C |
| ATOM | 7119 | OD1 | ASN | A | 473 | 28.533 | 64.653 | 63.201 | 1.00 | 72.56 | O |
| ATOM | 7120 | ND2 | ASN | A | 473 | 26.517 | 64.702 | 64.181 | 1.00 | 70.68 | N |
| ATOM | 7123 | C | ASN | A | 473 | 27.912 | 62.992 | 60.665 | 1.00 | 66.37 | C |
| ATOM | 7124 | O | ASN | A | 473 | 27.616 | 63.960 | 60.011 | 1.00 | 68.02 | O |
| ATOM | 7125 | N | ALA | A | 474 | 29.133 | 62.452 | 60.621 | 1.00 | 65.12 | N |
| ATOM | 7127 | CA | ALA | A | 474 | 30.220 | 63.060 | 59.830 | 1.00 | 65.88 | C |
| ATOM | 7129 | CB | ALA | A | 474 | 31.554 | 62.342 | 60.019 | 1.00 | 62.40 | C |
| ATOM | 7133 | C | ALA | A | 474 | 29.778 | 62.956 | 58.411 | 1.00 | 66.42 | C |
| ATOM | 7134 | O | ALA | A | 474 | 29.829 | 63.943 | 57.670 | 1.00 | 69.09 | O |
| ATOM | 7135 | N | CYS | A | 475 | 29.294 | 61.756 | 58.068 | 1.00 | 64.28 | N |
| ATOM | 7137 | CA | CYS | A | 475 | 28.846 | 61.446 | 56.715 | 1.00 | 64.60 | C |
| ATOM | 7139 | CB | CYS | A | 475 | 28.276 | 60.046 | 56.655 | 1.00 | 61.39 | C |
| ATOM | 7142 | SG | CYS | A | 475 | 29.609 | 58.840 | 57.005 | 1.00 | 61.67 | S |
| ATOM | 7143 | C | CYS | A | 475 | 27.823 | 62.441 | 56.247 | 1.00 | 68.38 | C |
| ATOM | 7144 | O | CYS | A | 475 | 27.945 | 62.918 | 55.133 | 1.00 | 70.86 | O |
| ATOM | 7145 | N | ALA | A | 476 | 26.839 | 62.773 | 57.096 | 1.00 | 69.69 | N |
| ATOM | 7147 | CA | ALA | A | 476 | 25.739 | 63.659 | 56.708 | 1.00 | 73.44 | C |
| ATOM | 7149 | CB | ALA | A | 476 | 24.512 | 63.632 | 57.733 | 1.00 | 74.87 | C |
| ATOM | 7153 | C | ALA | A | 476 | 26.315 | 65.042 | 56.588 | 1.00 | 76.96 | C |
| ATOM | 7154 | O | ALA | A | 476 | 25.922 | 65.815 | 55.746 | 1.00 | 80.28 | O |
| ATOM | 7155 | N | ALA | A | 477 | 27.284 | 65.326 | 57.441 | 1.00 | 76.83 | N |
| ATOM | 7157 | CA | ALA | A | 477 | 27.946 | 66.610 | 57.480 | 1.00 | 79.93 | C |
| ATOM | 7159 | CB | ALA | A | 477 | 28.692 | 66.746 | 58.841 | 1.00 | 79.17 | C |
| ATOM | 7163 | C | ALA | A | 477 | 28.883 | 66.854 | 56.259 | 1.00 | 80.17 | C |
| ATOM | 7164 | O | ALA | A | 477 | 29.420 | 67.924 | 56.119 | 1.00 | 83.71 | O |
| ATOM | 7165 | N | THR | A | 478 | 29.061 | 65.887 | 55.367 | 1.00 | 77.44 | N |
| ATOM | 7167 | CA | THR | A | 478 | 29.857 | 66.103 | 54.148 | 1.00 | 78.09 | C |
| ATOM | 7169 | CB | THR | A | 478 | 31.207 | 65.328 | 54.184 | 1.00 | 74.61 | C |
| ATOM | 7171 | OG1 | THR | A | 478 | 31.657 | 65.181 | 55.522 | 1.00 | 72.75 | O |
| ATOM | 7173 | CG2 | THR | A | 478 | 32.284 | 66.181 | 53.526 | 1.00 | 77.14 | C |
| ATOM | 7177 | C | THR | A | 478 | 29.159 | 65.664 | 52.870 | 1.00 | 77.93 | C |
| ATOM | 7178 | O | THR | A | 478 | 29.821 | 65.477 | 51.834 | 1.00 | 78.45 | O |
| ATOM | 7179 | N | GLY | A | 479 | 27.857 | 65.436 | 52.920 | 1.00 | 77.46 | N |
| ATOM | 7181 | CA | GLY | A | 479 | 27.162 | 64.913 | 51.748 | 1.00 | 77.03 | C |
| ATOM | 7184 | C | GLY | A | 479 | 27.510 | 63.493 | 51.309 | 1.00 | 71.99 | C |
| ATOM | 7185 | O | GLY | A | 479 | 27.172 | 63.096 | 50.232 | 1.00 | 71.57 | O |
| ATOM | 7186 | N | GLN | A | 480 | 28.197 | 62.715 | 52.127 | 1.00 | 68.57 | N |
| ATOM | 7188 | CA | GLN | A | 480 | 28.453 | 61.317 | 51.786 | 1.00 | 64.64 | C |
| ATOM | 7190 | CB | GLN | A | 480 | 29.784 | 60.854 | 52.334 | 1.00 | 61.62 | C |
| ATOM | 7193 | CG | GLN | A | 480 | 30.980 | 61.814 | 52.059 | 1.00 | 64.83 | C |
| ATOM | 7196 | CD | GLN | A | 480 | 32.316 | 61.198 | 52.592 | 1.00 | 64.82 | C |
| ATOM | 7197 | OE1 | GLN | A | 480 | 32.487 | 61.054 | 53.819 | 1.00 | 64.21 | O |
| ATOM | 7198 | NE2 | GLN | A | 480 | 33.224 | 60.787 | 51.671 | 1.00 | 62.23 | N |
| ATOM | 7201 | C | GLN | A | 480 | 27.227 | 60.525 | 52.300 | 1.00 | 63.37 | C |
| ATOM | 7202 | O | GLN | A | 480 | 27.260 | 59.788 | 53.314 | 1.00 | 61.12 | O |
| ATOM | 7203 | N | VAL | A | 481 | 26.162 | 60.692 | 51.514 | 1.00 | 64.68 | N |
| ATOM | 7205 | CA | VAL | A | 481 | 24.809 | 60.368 | 51.872 | 1.00 | 65.06 | C |
| ATOM | 7207 | CB | VAL | A | 481 | 24.167 | 61.693 | 52.392 | 1.00 | 69.11 | C |
| ATOM | 7209 | CG1 | VAL | A | 481 | 22.737 | 61.873 | 51.999 | 1.00 | 71.00 | C |
| ATOM | 7213 | CG2 | VAL | A | 481 | 24.349 | 61.727 | 53.885 | 1.00 | 68.55 | C |
| ATOM | 7217 | C | VAL | A | 481 | 24.142 | 59.795 | 50.629 | 1.00 | 65.55 | C |
| ATOM | 7218 | O | VAL | A | 481 | 24.589 | 60.047 | 49.535 | 1.00 | 66.48 | O |
| ATOM | 7219 | N | CYS | A | 482 | 23.053 | 59.055 | 50.768 | 1.00 | 69.26 | N |
| ATOM | 7221 | CA | CYS | A | 482 | 22.381 | 58.508 | 49.580 | 1.00 | 68.72 | C |
| ATOM | 7223 | CB | CYS | A | 482 | 21.207 | 57.639 | 49.988 | 1.00 | 67.54 | C |
| ATOM | 7226 | SG | CYS | A | 482 | 21.874 | 56.056 | 50.630 | 1.00 | 70.71 | S |
| ATOM | 7227 | C | CYS | A | 482 | 21.898 | 59.552 | 48.623 | 1.00 | 69.13 | C |
| ATOM | 7228 | O | CYS | A | 482 | 21.270 | 60.487 | 49.030 | 1.00 | 70.33 | O |
| ATOM | 7229 | N | HIS | A | 483 | 22.230 | 59.344 | 47.354 | 1.00 | 68.81 | N |
| ATOM | 7231 | CA | HIS | A | 483 | 21.865 | 60.191 | 46.211 | 1.00 | 69.88 | C |
| ATOM | 7233 | CB | HIS | A | 483 | 22.206 | 59.481 | 44.903 | 1.00 | 68.48 | C |
| ATOM | 7236 | CG | HIS | A | 483 | 22.273 | 60.355 | 43.700 | 1.00 | 69.95 | C |
| ATOM | 7237 | ND1 | HIS | A | 483 | 23.344 | 60.319 | 42.843 | 1.00 | 71.52 | N |
| ATOM | 7239 | CE1 | HIS | A | 483 | 23.138 | 61.154 | 41.840 | 1.00 | 72.59 | C |
| ATOM | 7241 | NE2 | HIS | A | 483 | 21.952 | 61.697 | 41.998 | 1.00 | 71.71 | N |
| ATOM | 7243 | CD2 | HIS | A | 483 | 21.383 | 61.207 | 43.144 | 1.00 | 70.25 | C |
| ATOM | 7245 | C | HIS | A | 483 | 20.394 | 60.442 | 46.219 | 1.00 | 70.55 | C |
| ATOM | 7246 | O | HIS | A | 483 | 19.633 | 59.603 | 46.639 | 1.00 | 69.08 | O |
| ATOM | 7247 | N | ALA | A | 484 | 20.033 | 61.644 | 45.777 | 1.00 | 73.54 | N |
| ATOM | 7249 | CA | ALA | A | 484 | 18.667 | 62.058 | 45.466 | 1.00 | 74.84 | C |
| ATOM | 7251 | CB | ALA | A | 484 | 18.715 | 63.282 | 44.604 | 1.00 | 77.22 | C |
| ATOM | 7255 | C | ALA | A | 484 | 17.804 | 60.997 | 44.766 | 1.00 | 73.78 | C |
| ATOM | 7256 | O | ALA | A | 484 | 16.634 | 60.816 | 45.150 | 1.00 | 74.54 | O |
| ATOM | 7257 | N | LEU | A | 485 | 18.360 | 60.294 | 43.758 | 1.00 | 72.45 | N |
| ATOM | 7259 | CA | LEU | A | 485 | 17.549 | 59.450 | 42.876 | 1.00 | 70.78 | C |
| ATOM | 7261 | CB | LEU | A | 485 | 18.233 | 59.189 | 41.526 | 1.00 | 69.85 | C |
| ATOM | 7264 | CG | LEU | A | 485 | 18.417 | 60.352 | 40.554 | 1.00 | 71.18 | C |
| ATOM | 7266 | CD1 | LEU | A | 485 | 19.226 | 59.942 | 39.360 | 1.00 | 68.77 | C |
| ATOM | 7270 | CD2 | LEU | A | 485 | 17.087 | 60.885 | 40.150 | 1.00 | 71.99 | C |
| ATOM | 7274 | C | LEU | A | 485 | 17.292 | 58.146 | 43.572 | 1.00 | 68.77 | C |
| ATOM | 7275 | O | LEU | A | 485 | 16.758 | 57.226 | 42.968 | 1.00 | 67.85 | O |
| ATOM | 7276 | N | CYS | A | 486 | 17.658 | 58.059 | 44.846 | 1.00 | 68.16 | N |
| ATOM | 7278 | CA | CYS | A | 486 | 17.418 | 56.844 | 45.588 | 1.00 | 66.85 | C |
| ATOM | 7280 | CB | CYS | A | 486 | 18.531 | 56.547 | 46.571 | 1.00 | 66.88 | C |
| ATOM | 7283 | SG | CYS | A | 486 | 20.068 | 56.462 | 45.781 | 1.00 | 68.62 | S |
| ATOM | 7284 | C | CYS | A | 486 | 16.216 | 56.889 | 46.426 | 1.00 | 66.34 | C |
| ATOM | 7285 | O | CYS | A | 486 | 15.944 | 57.829 | 47.074 | 1.00 | 66.83 | O |
| ATOM | 7286 | N | SER | A | 487 | 15.563 | 55.767 | 46.427 | 1.00 | 65.87 | N |
| ATOM | 7288 | CA | SER | A | 487 | 14.601 | 55.373 | 47.382 | 1.00 | 67.61 | C |
| ATOM | 7290 | CB | SER | A | 487 | 14.455 | 53.894 | 47.096 | 1.00 | 66.72 | C |
| ATOM | 7293 | OG | SER | A | 487 | 13.753 | 53.245 | 48.093 | 1.00 | 71.71 | O |
| ATOM | 7295 | C | SER | A | 487 | 15.053 | 55.580 | 48.835 | 1.00 | 68.27 | C |
| ATOM | 7296 | O | SER | A | 487 | 16.270 | 55.700 | 49.159 | 1.00 | 68.64 | O |
| ATOM | 7297 | N | PRO | A | 488 | 14.094 | 55.577 | 49.752 | 1.00 | 69.67 | N |
| ATOM | 7298 | CA | PRO | A | 488 | 14.443 | 55.564 | 51.181 | 1.00 | 69.32 | C |
| ATOM | 7300 | CB | PRO | A | 488 | 13.149 | 56.052 | 51.860 | 1.00 | 71.17 | C |
| ATOM | 7303 | CG | PRO | A | 488 | 12.071 | 55.622 | 50.953 | 1.00 | 72.01 | C |
| ATOM | 7306 | CD | PRO | A | 488 | 12.636 | 55.635 | 49.538 | 1.00 | 71.29 | C |
| ATOM | 7309 | C | PRO | A | 488 | 14.869 | 54.179 | 51.626 | 1.00 | 67.39 | C |
| ATOM | 7310 | O | PRO | A | 488 | 14.883 | 53.915 | 52.804 | 1.00 | 67.71 | O |
| ATOM | 7311 | N | GLU | A | 489 | 15.262 | 53.315 | 50.688 | 1.00 | 66.05 | N |
| ATOM | 7313 | CA | GLU | A | 489 | 15.650 | 51.931 | 50.990 | 1.00 | 64.34 | C |
| ATOM | 7315 | CB | GLU | A | 489 | 14.944 | 51.012 | 49.979 | 1.00 | 64.65 | C |
| ATOM | 7318 | CG | GLU | A | 489 | 13.950 | 49.987 | 50.564 | 1.00 | 66.51 | C |
| ATOM | 7321 | CD | GLU | A | 489 | 13.583 | 48.907 | 49.525 | 1.00 | 69.58 | C |
| ATOM | 7322 | OE1 | GLU | A | 489 | 13.820 | 47.707 | 49.835 | 1.00 | 69.93 | O |
| ATOM | 7323 | OE2 | GLU | A | 489 | 13.131 | 49.249 | 48.381 | 1.00 | 70.48 | O |
| ATOM | 7324 | C | GLU | A | 489 | 17.197 | 51.761 | 50.970 | 1.00 | 62.02 | C |
| ATOM | 7325 | O | GLU | A | 489 | 17.725 | 50.641 | 50.992 | 1.00 | 60.23 | O |
| ATOM | 7326 | N | GLY | A | 490 | 17.902 | 52.885 | 50.883 | 1.00 | 61.19 | N |
| ATOM | 7328 | CA | GLY | A | 490 | 19.311 | 52.942 | 51.176 | 1.00 | 60.24 | C |
| ATOM | 7331 | C | GLY | A | 490 | 20.262 | 52.839 | 50.001 | 1.00 | 59.52 | C |
| ATOM | 7332 | O | GLY | A | 490 | 19.800 | 52.615 | 48.891 | 1.00 | 59.65 | O |
| ATOM | 7333 | N | CYS | A | 491 | 21.577 | 52.973 | 50.241 | 1.00 | 58.69 | N |
| ATOM | 7335 | CA | CYS | A | 491 | 22.579 | 52.834 | 49.190 | 1.00 | 58.30 | C |
| ATOM | 7337 | CB | CYS | A | 491 | 23.088 | 54.159 | 48.688 | 1.00 | 59.18 | C |
| ATOM | 7340 | SG | CYS | A | 491 | 23.678 | 55.247 | 49.964 | 1.00 | 59.51 | S |
| ATOM | 7341 | C | CYS | A | 491 | 23.768 | 52.133 | 49.698 | 1.00 | 58.17 | C |
| ATOM | 7342 | O | CYS | A | 491 | 23.906 | 52.025 | 50.870 | 1.00 | 60.09 | O |
| ATOM | 7343 | N | TRP | A | 492 | 24.644 | 51.722 | 48.785 | 1.00 | 57.47 | N |
| ATOM | 7345 | CA | TRP | A | 492 | 25.960 | 51.167 | 49.054 | 1.00 | 57.58 | C |
| ATOM | 7347 | CB | TRP | A | 492 | 26.198 | 49.914 | 48.208 | 1.00 | 57.23 | C |
| ATOM | 7350 | CG | TRP | A | 492 | 25.251 | 48.846 | 48.427 | 1.00 | 56.66 | C |
| ATOM | 7351 | CD1 | TRP | A | 492 | 24.125 | 48.616 | 47.769 | 1.00 | 56.12 | C |
| ATOM | 7353 | NE1 | TRP | A | 492 | 23.504 | 47.505 | 48.257 | 1.00 | 56.13 | N |
| ATOM | 7355 | CE2 | TRP | A | 492 | 24.257 | 47.030 | 49.275 | 1.00 | 56.67 | C |
| ATOM | 7356 | CD2 | TRP | A | 492 | 25.367 | 47.843 | 49.384 | 1.00 | 56.95 | C |
| ATOM | 7357 | CE3 | TRP | A | 492 | 26.323 | 47.539 | 50.332 | 1.00 | 57.68 | C |
| ATOM | 7359 | CZ3 | TRP | A | 492 | 26.133 | 46.504 | 51.114 | 1.00 | 58.09 | C |
| ATOM | 7361 | CH2 | TRP | A | 492 | 25.012 | 45.719 | 50.987 | 1.00 | 57.91 | C |
| ATOM | 7363 | CZ2 | TRP | A | 492 | 24.068 | 45.969 | 50.056 | 1.00 | 57.20 | C |
| ATOM | 7365 | C | TRP | A | 492 | 26.997 | 52.155 | 48.596 | 1.00 | 58.58 | C |
| ATOM | 7366 | O | TRP | A | 492 | 28.024 | 51.764 | 48.120 | 1.00 | 59.09 | O |
| ATOM | 7367 | N | GLY | A | 493 | 26.736 | 53.443 | 48.717 | 1.00 | 59.23 | N |
| ATOM | 7369 | CA | GLY | A | 493 | 27.584 | 54.415 | 48.045 | 1.00 | 60.57 | C |
| ATOM | 7372 | C | GLY | A | 493 | 26.905 | 55.667 | 47.512 | 1.00 | 61.34 | C |
| ATOM | 7373 | O | GLY | A | 493 | 25.700 | 55.817 | 47.542 | 1.00 | 60.80 | O |
| ATOM | 7374 | N | PRO | A | 494 | 27.715 | 56.602 | 47.067 | 1.00 | 63.04 | N |
| ATOM | 7375 | CA | PRO | A | 494 | 27.223 | 57.926 | 46.798 | 1.00 | 65.24 | C |
| ATOM | 7377 | CB | PRO | A | 494 | 28.514 | 58.706 | 46.562 | 1.00 | 67.15 | C |
| ATOM | 7380 | CG | PRO | A | 494 | 29.467 | 57.738 | 46.091 | 1.00 | 66.26 | C |
| ATOM | 7383 | CD | PRO | A | 494 | 29.148 | 56.516 | 46.802 | 1.00 | 64.20 | C |
| ATOM | 7386 | C | PRO | A | 494 | 26.328 | 57.929 | 45.573 | 1.00 | 66.33 | C |
| ATOM | 7387 | O | PRO | A | 494 | 25.317 | 58.597 | 45.559 | 1.00 | 68.00 | O |
| ATOM | 7388 | N | GLU | A | 495 | 26.684 | 57.109 | 44.592 | 1.00 | 66.87 | N |
| ATOM | 7390 | CA | GLU | A | 495 | 26.191 | 57.194 | 43.228 | 1.00 | 67.29 | C |
| ATOM | 7392 | CB | GLU | A | 495 | 27.262 | 56.599 | 42.343 | 1.00 | 67.64 | C |
| ATOM | 7395 | CG | GLU | A | 495 | 28.401 | 57.573 | 42.148 | 1.00 | 71.68 | C |
| ATOM | 7398 | CD | GLU | A | 495 | 29.577 | 56.927 | 41.440 | 1.00 | 76.82 | C |
| ATOM | 7399 | OE1 | GLU | A | 495 | 29.470 | 55.740 | 40.951 | 1.00 | 75.08 | O |
| ATOM | 7400 | OE2 | GLU | A | 495 | 30.632 | 57.625 | 41.377 | 1.00 | 82.21 | O |
| ATOM | 7401 | C | GLU | A | 495 | 24.837 | 56.544 | 42.919 | 1.00 | 65.80 | C |
| ATOM | 7402 | O | GLU | A | 495 | 24.367 | 55.692 | 43.676 | 1.00 | 63.46 | O |
| ATOM | 7403 | N | PRO | A | 496 | 24.192 | 56.963 | 41.810 | 1.00 | 67.18 | N |
| ATOM | 7404 | CA | PRO | A | 496 | 22.872 | 56.413 | 41.435 | 1.00 | 65.83 | C |
| ATOM | 7406 | CB | PRO | A | 496 | 22.428 | 57.256 | 40.256 | 1.00 | 66.55 | C |
| ATOM | 7409 | CG | PRO | A | 496 | 23.607 | 57.978 | 39.815 | 1.00 | 69.03 | C |
| ATOM | 7412 | CD | PRO | A | 496 | 24.636 | 58.013 | 40.872 | 1.00 | 69.18 | C |
| ATOM | 7415 | C | PRO | A | 496 | 22.869 | 54.918 | 41.127 | 1.00 | 64.10 | C |
| ATOM | 7416 | O | PRO | A | 496 | 21.872 | 54.322 | 41.478 | 1.00 | 64.50 | O |
| ATOM | 7417 | N | ARG | A | 497 | 23.927 | 54.318 | 40.585 | 1.00 | 63.43 | N |
| ATOM | 7419 | CA | ARG | A | 497 | 23.998 | 52.856 | 40.554 | 1.00 | 61.81 | C |
| ATOM | 7421 | CB | ARG | A | 497 | 25.102 | 52.371 | 39.600 | 1.00 | 62.79 | C |
| ATOM | 7424 | CG | ARG | A | 497 | 26.384 | 53.144 | 39.609 | 1.00 | 64.56 | C |
| ATOM | 7427 | CD | ARG | A | 497 | 27.644 | 52.336 | 39.155 | 1.00 | 65.35 | C |
| ATOM | 7430 | NE | ARG | A | 497 | 28.741 | 52.771 | 40.030 | 1.00 | 68.46 | N |
| ATOM | 7432 | CZ | ARG | A | 497 | 30.025 | 52.441 | 39.960 | 1.00 | 68.79 | C |
| ATOM | 7433 | NH1 | ARG | A | 497 | 30.472 | 51.636 | 39.000 | 1.00 | 68.43 | N |
| ATOM | 7436 | NH2 | ARG | A | 497 | 30.864 | 52.943 | 40.888 | 1.00 | 69.25 | N |
| ATOM | 7439 | C | ARG | A | 497 | 24.213 | 52.199 | 41.913 | 1.00 | 60.94 | C |
| ATOM | 7440 | O | ARG | A | 497 | 24.213 | 50.997 | 42.032 | 1.00 | 60.39 | O |
| ATOM | 7441 | N | ASP | A | 498 | 24.436 | 52.990 | 42.939 | 1.00 | 61.84 | N |
| ATOM | 7443 | CA | ASP | A | 498 | 24.624 | 52.484 | 44.285 | 1.00 | 60.98 | C |
| ATOM | 7445 | CB | ASP | A | 498 | 25.579 | 53.410 | 45.067 | 1.00 | 62.53 | C |
| ATOM | 7448 | CG | ASP | A | 498 | 27.019 | 53.424 | 44.503 | 1.00 | 62.37 | C |
| ATOM | 7449 | OD1 | ASP | A | 498 | 27.333 | 52.507 | 43.754 | 1.00 | 63.21 | O |
| ATOM | 7450 | OD2 | ASP | A | 498 | 27.902 | 54.266 | 44.765 | 1.00 | 61.45 | O |
| ATOM | 7451 | C | ASP | A | 498 | 23.314 | 52.291 | 45.059 | 1.00 | 60.30 | C |
| ATOM | 7452 | O | ASP | A | 498 | 23.324 | 51.685 | 46.104 | 1.00 | 58.31 | O |
| ATOM | 7453 | N | CYS | A | 499 | 22.172 | 52.740 | 44.543 | 1.00 | 61.23 | N |
| ATOM | 7455 | CA | CYS | A | 499 | 20.943 | 52.486 | 45.291 | 1.00 | 61.84 | C |
| ATOM | 7457 | CB | CYS | A | 499 | 19.812 | 53.357 | 44.872 | 1.00 | 62.74 | C |
| ATOM | 7460 | SG | CYS | A | 499 | 20.331 | 54.978 | 44.411 | 1.00 | 66.84 | S |
| ATOM | 7461 | C | CYS | A | 499 | 20.433 | 51.076 | 45.244 | 1.00 | 61.36 | C |
| ATOM | 7462 | O | CYS | A | 499 | 20.878 | 50.243 | 44.460 | 1.00 | 61.72 | O |
| ATOM | 7463 | N | VAL | A | 500 | 19.488 | 50.844 | 46.136 | 1.00 | 61.48 | N |
| ATOM | 7465 | CA | VAL | A | 500 | 18.869 | 49.574 | 46.344 | 1.00 | 61.04 | C |
| ATOM | 7467 | CB | VAL | A | 500 | 18.561 | 49.388 | 47.868 | 1.00 | 61.98 | C |
| ATOM | 7469 | CG1 | VAL | A | 500 | 17.793 | 48.079 | 48.131 | 1.00 | 63.20 | C |
| ATOM | 7473 | CG2 | VAL | A | 500 | 19.840 | 49.433 | 48.728 | 1.00 | 60.20 | C |
| ATOM | 7477 | C | VAL | A | 500 | 17.588 | 49.573 | 45.554 | 1.00 | 61.08 | C |
| ATOM | 7478 | O | VAL | A | 500 | 17.157 | 48.532 | 45.141 | 1.00 | 61.21 | O |
| ATOM | 7479 | N | SER | A | 501 | 16.978 | 50.746 | 45.391 | 1.00 | 61.78 | N |
| ATOM | 7481 | CA | SER | A | 501 | 15.715 | 50.963 | 44.651 | 1.00 | 62.67 | C |
| ATOM | 7483 | CB | SER | A | 501 | 14.496 | 50.409 | 45.457 | 1.00 | 63.41 | C |
| ATOM | 7486 | OG | SER | A | 501 | 14.295 | 50.992 | 46.745 | 1.00 | 62.89 | O |
| ATOM | 7488 | C | SER | A | 501 | 15.580 | 52.495 | 44.276 | 1.00 | 64.49 | C |
| ATOM | 7489 | O | SER | A | 501 | 16.205 | 53.346 | 44.915 | 1.00 | 65.53 | O |
| ATOM | 7490 | N | CYS | A | 502 | 14.796 | 52.870 | 43.271 | 1.00 | 65.42 | N |
| ATOM | 7492 | CA | CYS | A | 502 | 14.828 | 54.267 | 42.789 | 1.00 | 67.75 | C |
| ATOM | 7494 | CB | CYS | A | 502 | 14.768 | 54.264 | 41.279 | 1.00 | 67.77 | C |
| ATOM | 7497 | SG | CYS | A | 502 | 16.221 | 53.363 | 40.726 | 1.00 | 74.29 | S |
| ATOM | 7498 | C | CYS | A | 502 | 13.720 | 55.123 | 43.312 | 1.00 | 68.44 | C |
| ATOM | 7499 | O | CYS | A | 502 | 12.717 | 54.556 | 43.656 | 1.00 | 69.58 | O |
| ATOM | 7500 | N | ALA | A | 503 | 13.838 | 56.455 | 43.368 | 1.00 | 68.92 | N |
| ATOM | 7502 | CA | ALA | A | 503 | 12.653 | 57.227 | 43.798 | 1.00 | 71.83 | C |
| ATOM | 7504 | CB | ALA | A | 503 | 12.941 | 58.787 | 44.045 | 1.00 | 73.74 | C |
| ATOM | 7508 | C | ALA | A | 503 | 11.611 | 57.004 | 42.683 | 1.00 | 72.91 | C |
| ATOM | 7509 | O | ALA | A | 503 | 10.457 | 56.658 | 42.934 | 1.00 | 72.96 | O |
| ATOM | 7510 | N | ASN | A | 504 | 12.089 | 57.121 | 41.445 | 1.00 | 72.99 | N |
| ATOM | 7512 | CA | ASN | A | 504 | 11.256 | 57.123 | 40.243 | 1.00 | 74.36 | C |
| ATOM | 7514 | CB | ASN | A | 504 | 11.575 | 58.361 | 39.406 | 1.00 | 75.32 | C |
| ATOM | 7517 | CG | ASN | A | 504 | 11.494 | 59.602 | 40.235 | 1.00 | 78.00 | C |
| ATOM | 7518 | OD1 | ASN | A | 504 | 10.412 | 60.228 | 40.359 | 1.00 | 82.73 | O |
| ATOM | 7519 | ND2 | ASN | A | 504 | 12.596 | 59.937 | 40.899 | 1.00 | 75.00 | N |
| ATOM | 7522 | C | ASN | A | 504 | 11.434 | 55.846 | 39.457 | 1.00 | 73.02 | C |
| ATOM | 7523 | O | ASN | A | 504 | 10.662 | 54.873 | 39.645 | 1.00 | 72.93 | O |
| ATOM | 7524 | N | VAL | A | 505 | 12.501 | 55.828 | 38.644 | 1.00 | 72.34 | N |
| ATOM | 7526 | CA | VAL | A | 505 | 12.729 | 54.790 | 37.645 | 1.00 | 70.30 | C |
| ATOM | 7528 | CB | VAL | A | 505 | 12.389 | 55.347 | 36.278 | 1.00 | 71.79 | C |
| ATOM | 7530 | CG1 | VAL | A | 505 | 12.524 | 54.251 | 35.263 | 1.00 | 71.94 | C |
| ATOM | 7534 | CG2 | VAL | A | 505 | 11.011 | 55.887 | 36.245 | 1.00 | 72.79 | C |
| ATOM | 7538 | C | VAL | A | 505 | 14.158 | 54.374 | 37.502 | 1.00 | 67.81 | C |
| ATOM | 7539 | O | VAL | A | 505 | 15.046 | 55.143 | 37.612 | 1.00 | 68.17 | O |
| ATOM | 7540 | N | SER | A | 506 | 14.376 | 53.149 | 37.128 | 1.00 | 66.94 | N |
| ATOM | 7542 | CA | SER | A | 506 | 15.736 | 52.664 | 36.881 | 1.00 | 65.51 | C |
| ATOM | 7544 | CB | SER | A | 506 | 15.987 | 51.363 | 37.643 | 1.00 | 63.70 | C |
| ATOM | 7547 | OG | SER | A | 506 | 14.880 | 50.489 | 37.521 | 1.00 | 63.05 | O |
| ATOM | 7549 | C | SER | A | 506 | 15.894 | 52.410 | 35.406 | 1.00 | 65.47 | C |
| ATOM | 7550 | O | SER | A | 506 | 14.922 | 52.084 | 34.730 | 1.00 | 65.31 | O |
| ATOM | 7551 | N | ARG | A | 507 | 17.104 | 52.648 | 34.920 | 1.00 | 66.02 | N |
| ATOM | 7553 | CA | ARG | A | 507 | 17.563 | 52.198 | 33.606 | 1.00 | 66.52 | C |
| ATOM | 7555 | CB | ARG | A | 507 | 18.321 | 53.306 | 32.922 | 1.00 | 67.29 | C |
| ATOM | 7558 | CG | ARG | A | 507 | 18.520 | 53.085 | 31.458 | 1.00 | 67.04 | C |
| ATOM | 7561 | CD | ARG | A | 507 | 19.211 | 54.254 | 30.845 | 1.00 | 69.49 | C |
| ATOM | 7564 | NE | ARG | A | 507 | 18.350 | 55.355 | 30.381 | 1.00 | 72.58 | N |
| ATOM | 7566 | CZ | ARG | A | 507 | 18.876 | 56.525 | 29.999 | 1.00 | 77.80 | C |
| ATOM | 7567 | NH1 | ARG | A | 507 | 20.241 | 56.684 | 30.043 | 1.00 | 76.65 | N |
| ATOM | 7570 | NH2 | ARG | A | 507 | 18.076 | 57.514 | 29.521 | 1.00 | 79.53 | N |
| ATOM | 7573 | C | ARG | A | 507 | 18.500 | 51.002 | 33.751 | 1.00 | 66.50 | C |
| ATOM | 7574 | O | ARG | A | 507 | 19.747 | 51.161 | 33.656 | 1.00 | 66.13 | O |
| ATOM | 7575 | N | GLY | A | 508 | 17.900 | 49.833 | 34.047 | 1.00 | 66.65 | N |
| ATOM | 7577 | CA | GLY | A | 508 | 18.644 | 48.603 | 34.086 | 1.00 | 65.74 | C |
| ATOM | 7580 | C | GLY | A | 508 | 19.525 | 48.460 | 35.303 | 1.00 | 65.94 | C |
| ATOM | 7581 | O | GLY | A | 508 | 19.625 | 47.337 | 35.840 | 1.00 | 66.96 | O |
| ATOM | 7582 | N | ARG | A | 509 | 20.223 | 49.516 | 35.730 | 1.00 | 66.56 | N |
| ATOM | 7584 | CA | ARG | A | 509 | 21.083 | 49.445 | 36.964 | 1.00 | 66.64 | C |
| ATOM | 7586 | CB | ARG | A | 509 | 22.489 | 48.881 | 36.701 | 1.00 | 66.71 | C |
| ATOM | 7589 | CG | ARG | A | 509 | 23.298 | 49.663 | 35.556 | 1.00 | 69.61 | C |
| ATOM | 7592 | CD | ARG | A | 509 | 23.416 | 48.867 | 34.185 | 1.00 | 70.79 | C |
| ATOM | 7595 | NE | ARG | A | 509 | 24.374 | 49.468 | 33.244 | 1.00 | 73.28 | N |
| ATOM | 7597 | CZ | ARG | A | 509 | 25.628 | 48.985 | 32.971 | 1.00 | 75.15 | C |
| ATOM | 7598 | NH1 | ARG | A | 509 | 26.131 | 47.881 | 33.569 | 1.00 | 71.78 | N |
| ATOM | 7601 | NH2 | ARG | A | 509 | 26.402 | 49.636 | 32.074 | 1.00 | 77.07 | N |
| ATOM | 7604 | C | ARG | A | 509 | 21.264 | 50.782 | 37.627 | 1.00 | 66.73 | C |
| ATOM | 7605 | O | ARG | A | 509 | 21.410 | 50.847 | 38.811 | 1.00 | 67.34 | O |
| ATOM | 7606 | N | GLU | A | 510 | 21.297 | 51.853 | 36.858 | 1.00 | 67.08 | N |
| ATOM | 7608 | CA | GLU | A | 510 | 21.459 | 53.183 | 37.434 | 1.00 | 67.32 | C |
| ATOM | 7610 | CB | GLU | A | 510 | 22.246 | 54.101 | 36.478 | 1.00 | 68.74 | C |
| ATOM | 7613 | CG | GLU | A | 510 | 21.936 | 55.585 | 36.586 | 1.00 | 70.35 | C |
| ATOM | 7616 | CD | GLU | A | 510 | 22.933 | 56.425 | 35.809 | 1.00 | 74.79 | C |
| ATOM | 7617 | OE1 | GLU | A | 510 | 23.986 | 55.840 | 35.426 | 1.00 | 74.61 | O |
| ATOM | 7618 | OE2 | GLU | A | 510 | 22.658 | 57.652 | 35.572 | 1.00 | 77.88 | O |
| ATOM | 7619 | C | GLU | A | 510 | 20.078 | 53.729 | 37.693 | 1.00 | 66.81 | C |
| ATOM | 7620 | O | GLU | A | 510 | 19.137 | 53.401 | 36.977 | 1.00 | 66.94 | O |
| ATOM | 7621 | N | CYS | A | 511 | 19.959 | 54.554 | 38.719 | 1.00 | 66.39 | N |
| ATOM | 7623 | CA | CYS | A | 511 | 18.693 | 55.135 | 39.066 | 1.00 | 66.14 | C |
| ATOM | 7625 | CB | CYS | A | 511 | 18.610 | 55.343 | 40.548 | 1.00 | 65.95 | C |
| ATOM | 7628 | SG | CYS | A | 511 | 18.160 | 53.799 | 41.363 | 1.00 | 62.99 | S |
| ATOM | 7629 | C | CYS | A | 511 | 18.661 | 56.414 | 38.318 | 1.00 | 68.45 | C |
| ATOM | 7630 | O | CYS | A | 511 | 19.671 | 57.145 | 38.275 | 1.00 | 69.88 | O |
| ATOM | 7631 | N | VAL | A | 512 | 17.519 | 56.652 | 37.670 | 1.00 | 69.26 | N |
| ATOM | 7633 | CA | VAL | A | 512 | 17.284 | 57.840 | 36.860 | 1.00 | 71.01 | C |
| ATOM | 7635 | CB | VAL | A | 512 | 17.364 | 57.499 | 35.357 | 1.00 | 70.31 | C |
| ATOM | 7637 | CG1 | VAL | A | 512 | 17.763 | 58.724 | 34.563 | 1.00 | 74.73 | C |
| ATOM | 7641 | CG2 | VAL | A | 512 | 18.413 | 56.495 | 35.122 | 1.00 | 69.40 | C |
| ATOM | 7645 | C | VAL | A | 512 | 15.961 | 58.568 | 37.218 | 1.00 | 72.75 | C |
| ATOM | 7646 | O | VAL | A | 512 | 14.991 | 57.984 | 37.710 | 1.00 | 71.70 | O |
| ATOM | 7647 | N | ASP | A | 513 | 15.953 | 59.871 | 36.952 | 1.00 | 75.55 | N |
| ATOM | 7649 | CA | ASP | A | 513 | 14.763 | 60.722 | 37.132 | 1.00 | 78.34 | C |
| ATOM | 7651 | CB | ASP | A | 513 | 15.195 | 62.210 | 37.064 | 1.00 | 82.23 | C |
| ATOM | 7654 | CG | ASP | A | 513 | 15.778 | 62.604 | 35.666 | 1.00 | 86.47 | C |
| ATOM | 7655 | OD1 | ASP | A | 513 | 16.485 | 61.724 | 35.028 | 1.00 | 85.95 | O |
| ATOM | 7656 | OD2 | ASP | A | 513 | 15.527 | 63.750 | 35.143 | 1.00 | 89.04 | O |
| ATOM | 7657 | C | ASP | A | 513 | 13.600 | 60.486 | 36.136 | 1.00 | 78.00 | C |
| ATOM | 7658 | O | ASP | A | 513 | 12.407 | 60.618 | 36.490 | 1.00 | 78.71 | O |
| ATOM | 7659 | N | LYS | A | 514 | 13.936 | 60.184 | 34.885 | 1.00 | 76.63 | N |
| ATOM | 7661 | CA | LYS | A | 514 | 12.907 | 60.080 | 33.832 | 1.00 | 77.37 | C |
| ATOM | 7663 | CB | LYS | A | 514 | 12.661 | 61.438 | 33.090 | 1.00 | 79.82 | C |
| ATOM | 7670 | C | LYS | A | 514 | 13.389 | 59.087 | 32.841 | 1.00 | 74.72 | C |
| ATOM | 7671 | O | LYS | A | 514 | 14.568 | 58.916 | 32.682 | 1.00 | 71.86 | O |
| ATOM | 7672 | N | CYS | A | 515 | 12.473 | 58.478 | 32.118 | 1.00 | 75.49 | N |
| ATOM | 7674 | CA | CYS | A | 515 | 12.882 | 57.736 | 30.931 | 1.00 | 75.27 | C |
| ATOM | 7676 | CB | CYS | A | 515 | 11.953 | 56.576 | 30.660 | 1.00 | 74.23 | C |
| ATOM | 7679 | SG | CYS | A | 515 | 12.029 | 55.503 | 32.049 | 1.00 | 75.96 | S |
| ATOM | 7680 | C | CYS | A | 515 | 12.878 | 58.608 | 29.723 | 1.00 | 77.45 | C |
| ATOM | 7681 | O | CYS | A | 515 | 12.396 | 59.736 | 29.762 | 1.00 | 80.72 | O |
| ATOM | 7682 | N | ASN | A | 516 | 13.375 | 58.047 | 28.632 | 1.00 | 76.28 | N |
| ATOM | 7684 | CA | ASN | A | 516 | 13.450 | 58.745 | 27.390 | 1.00 | 78.27 | C |
| ATOM | 7686 | CB | ASN | A | 516 | 14.682 | 58.263 | 26.628 | 1.00 | 76.82 | C |
| ATOM | 7689 | CG | ASN | A | 516 | 15.962 | 58.751 | 27.257 | 1.00 | 76.59 | C |
| ATOM | 7690 | OD1 | ASN | A | 516 | 15.913 | 59.562 | 28.158 | 1.00 | 76.10 | O |
| ATOM | 7691 | ND2 | ASN | A | 516 | 17.118 | 58.255 | 26.792 | 1.00 | 78.58 | N |
| ATOM | 7694 | C | ASN | A | 516 | 12.162 | 58.593 | 26.589 | 1.00 | 79.48 | C |
| ATOM | 7695 | O | ASN | A | 516 | 12.214 | 58.369 | 25.392 | 1.00 | 80.32 | O |
| ATOM | 7696 | N | LEU | A | 517 | 11.005 | 58.722 | 27.247 | 1.00 | 80.26 | N |
| ATOM | 7698 | CA | LEU | A | 517 | 9.729 | 58.866 | 26.540 | 1.00 | 81.89 | C |
| ATOM | 7700 | CB | LEU | A | 517 | 8.536 | 58.802 | 27.508 | 1.00 | 82.29 | C |
| ATOM | 7703 | CG | LEU | A | 517 | 8.472 | 57.763 | 28.612 | 1.00 | 78.11 | C |
| ATOM | 7705 | CD1 | LEU | A | 517 | 7.256 | 57.874 | 29.493 | 1.00 | 78.46 | C |
| ATOM | 7709 | CD2 | LEU | A | 517 | 8.494 | 56.410 | 27.960 | 1.00 | 77.57 | C |
| ATOM | 7713 | C | LEU | A | 517 | 9.787 | 60.258 | 25.953 | 1.00 | 85.89 | C |
| ATOM | 7714 | O | LEU | A | 517 | 9.879 | 61.252 | 26.730 | 1.00 | 89.33 | O |
| ATOM | 7715 | N | LEU | A | 518 | 9.773 | 60.389 | 24.633 | 1.00 | 86.14 | N |
| ATOM | 7717 | CA | LEU | A | 518 | 9.569 | 61.731 | 24.037 | 1.00 | 89.84 | C |
| ATOM | 7719 | CB | LEU | A | 518 | 8.620 | 62.609 | 24.906 | 1.00 | 92.23 | C |
| ATOM | 7722 | CG | LEU | A | 518 | 7.226 | 62.059 | 25.263 | 1.00 | 91.47 | C |
| ATOM | 7724 | CD1 | LEU | A | 518 | 6.556 | 62.839 | 26.352 | 1.00 | 93.70 | C |
| ATOM | 7728 | CD2 | LEU | A | 518 | 6.382 | 62.115 | 24.048 | 1.00 | 93.66 | C |
| ATOM | 7732 | C | LEU | A | 518 | 10.827 | 62.542 | 23.724 | 1.00 | 90.91 | C |
| ATOM | 7733 | O | LEU | A | 518 | 10.733 | 63.588 | 23.093 | 1.00 | 94.73 | O |
| ATOM | 7734 | N | GLU | A | 519 | 11.990 | 62.050 | 24.106 | 1.00 | 88.21 | N |
| ATOM | 7736 | CA | GLU | A | 519 | 13.211 | 62.857 | 24.134 | 1.00 | 90.11 | C |
| ATOM | 7738 | CB | GLU | A | 519 | 13.100 | 64.031 | 25.150 | 1.00 | 92.09 | C |
| ATOM | 7745 | C | GLU | A | 519 | 14.289 | 61.868 | 24.579 | 1.00 | 87.46 | C |
| ATOM | 7746 | O | GLU | A | 519 | 14.162 | 61.310 | 25.671 | 1.00 | 86.35 | O |
| ATOM | 7747 | N | GYL | A | 520 | 15.305 | 61.576 | 23.753 | 1.00 | 87.39 | N |
| ATOM | 7749 | CA | GYL | A | 520 | 16.367 | 60.664 | 24.168 | 1.00 | 84.28 | C |
| ATOM | 7752 | C | GYL | A | 520 | 17.184 | 59.916 | 23.123 | 1.00 | 83.49 | C |
| ATOM | 7753 | O | GYL | A | 520 | 16.762 | 59.776 | 21.962 | 1.00 | 83.56 | O |
| ATOM | 7754 | N | GLU | A | 521 | 18.343 | 59.402 | 23.598 | 1.00 | 82.41 | N |
| ATOM | 7756 | CA | GLU | A | 521 | 19.346 | 58.645 | 22.811 | 1.00 | 81.13 | C |
| ATOM | 7758 | CB | GLU | A | 521 | 20.537 | 58.131 | 23.652 | 1.00 | 79.67 | C |
| ATOM | 7765 | C | GLU | A | 521 | 18.600 | 57.538 | 22.157 | 1.00 | 78.75 | C |
| ATOM | 7766 | O | GLU | A | 521 | 18.095 | 57.788 | 21.071 | 1.00 | 81.59 | O |
| ATOM | 7767 | N | PRO | A | 522 | 18.450 | 56.371 | 22.786 | 1.00 | 75.18 | N |
| ATOM | 7768 | CA | PRO | A | 522 | 17.393 | 55.446 | 22.365 | 1.00 | 72.91 | C |
| ATOM | 7770 | CB | PRO | A | 522 | 17.911 | 54.074 | 22.846 | 1.00 | 70.53 | C |
| ATOM | 7773 | CG | PRO | A | 522 | 18.755 | 54.376 | 24.063 | 1.00 | 70.29 | C |
| ATOM | 7776 | CD | PRO | A | 522 | 19.206 | 55.822 | 23.940 | 1.00 | 73.90 | C |
| ATOM | 7779 | C | PRO | A | 522 | 16.098 | 55.838 | 23.085 | 1.00 | 72.50 | C |
| ATOM | 7780 | O | PRO | A | 522 | 16.099 | 56.091 | 24.277 | 1.00 | 71.74 | O |
| ATOM | 7781 | N | ARG | A | 523 | 15.000 | 55.914 | 22.354 | 1.00 | 73.17 | N |
| ATOM | 7783 | CA | ARG | A | 523 | 13.700 | 56.209 | 22.950 | 1.00 | 73.74 | C |
| ATOM | 7785 | CB | ARG | A | 523 | 12.642 | 56.629 | 21.883 | 1.00 | 75.26 | C |
| ATOM | 7794 | C | ARG | A | 523 | 13.348 | 54.933 | 23.704 | 1.00 | 70.93 | C |
| ATOM | 7795 | O | ARG | A | 523 | 13.763 | 53.837 | 23.330 | 1.00 | 68.98 | O |
| ATOM | 7796 | N | GLU | A | 524 | 12.655 | 55.074 | 24.817 | 1.00 | 71.17 | N |
| ATOM | 7798 | CA | GLU | A | 524 | 12.390 | 53.915 | 25.690 | 1.00 | 69.50 | C |
| ATOM | 7800 | CB | GLU | A | 524 | 13.381 | 53.846 | 26.934 | 1.00 | 67.31 | C |
| ATOM | 7807 | C | GLU | A | 524 | 10.900 | 53.981 | 26.076 | 1.00 | 70.75 | C |
| ATOM | 7808 | O | GLU | A | 524 | 10.278 | 55.027 | 25.933 | 1.00 | 72.03 | O |
| ATOM | 7809 | N | PHE | A | 525 | 10.339 | 52.834 | 26.487 | 1.00 | 70.41 | N |
| ATOM | 7811 | CA | PHE | A | 525 | 9.073 | 52.756 | 27.238 | 1.00 | 71.56 | C |
| ATOM | 7813 | CB | PHE | A | 525 | 8.072 | 51.852 | 26.513 | 1.00 | 71.53 | C |
| ATOM | 7816 | CG | PHE | A | 525 | 8.376 | 50.386 | 26.625 | 1.00 | 68.87 | C |
| ATOM | 7817 | CD1 | PHE | A | 525 | 7.672 | 49.582 | 27.527 | 1.00 | 66.88 | C |
| ATOM | 7819 | CE1 | PHE | A | 525 | 7.933 | 48.262 | 27.624 | 1.00 | 65.64 | C |
| ATOM | 7821 | CZ | PHE | A | 525 | 8.923 | 47.692 | 26.821 | 1.00 | 65.09 | C |
| ATOM | 7823 | CE2 | PHE | A | 525 | 9.625 | 48.470 | 25.920 | 1.00 | 64.47 | C |
| ATOM | 7825 | CD2 | PHE | A | 525 | 9.348 | 49.810 | 25.824 | 1.00 | 66.17 | C |
| ATOM | 7827 | C | PHE | A | 525 | 9.370 | 52.267 | 28.663 | 1.00 | 70.74 | C |
| ATOM | 7828 | O | PHE | A | 525 | 10.511 | 52.060 | 29.014 | 1.00 | 69.95 | O |
| ATOM | 7829 | N | VAL | A | 526 | 8.350 | 52.071 | 29.478 | 1.00 | 72.42 | N |
| ATOM | 7831 | CA | VAL | A | 526 | 8.512 | 51.804 | 30.902 | 1.00 | 71.94 | C |
| ATOM | 7833 | CB | VAL | A | 526 | 8.002 | 52.989 | 31.696 | 1.00 | 73.98 | C |
| ATOM | 7835 | CG1 | VAL | A | 526 | 7.837 | 52.617 | 33.185 | 1.00 | 75.09 | C |
| ATOM | 7839 | CG2 | VAL | A | 526 | 8.908 | 54.239 | 31.481 | 1.00 | 74.24 | C |
| ATOM | 7843 | C | VAL | A | 526 | 7.648 | 50.632 | 31.327 | 1.00 | 73.13 | C |
| ATOM | 7844 | O | VAL | A | 526 | 6.495 | 50.506 | 30.870 | 1.00 | 75.47 | O |
| ATOM | 7845 | N | GLU | A | 527 | 8.176 | 49.785 | 32.218 | 1.00 | 72.37 | N |
| ATOM | 7847 | CA | GLU | A | 527 | 7.356 | 48.786 | 32.900 | 1.00 | 73.28 | C |
| ATOM | 7849 | CB | GLU | A | 527 | 7.025 | 47.568 | 32.001 | 1.00 | 73.10 | C |
| ATOM | 7852 | CG | GLU | A | 527 | 8.218 | 46.727 | 31.521 | 1.00 | 71.94 | C |
| ATOM | 7855 | CD | GLU | A | 527 | 7.836 | 45.371 | 30.851 | 1.00 | 72.04 | C |
| ATOM | 7856 | OE1 | GLU | A | 527 | 6.743 | 45.307 | 30.202 | 1.00 | 69.38 | O |
| ATOM | 7857 | OE2 | GLU | A | 527 | 8.648 | 44.377 | 30.965 | 1.00 | 68.38 | O |
| ATOM | 7858 | C | GLU | A | 527 | 8.000 | 48.365 | 34.226 | 1.00 | 72.76 | C |
| ATOM | 7859 | O | GLU | A | 527 | 9.115 | 47.824 | 34.258 | 1.00 | 71.27 | O |
| ATOM | 7860 | N | ASN | A | 528 | 7.244 | 48.589 | 35.308 | 1.00 | 74.36 | N |
| ATOM | 7862 | CA | ASN | A | 528 | 7.661 | 48.313 | 36.678 | 1.00 | 73.60 | C |
| ATOM | 7864 | CB | ASN | A | 528 | 7.986 | 46.830 | 36.923 | 1.00 | 72.97 | C |
| ATOM | 7867 | CG | ASN | A | 528 | 7.226 | 45.878 | 35.978 | 1.00 | 75.77 | C |
| ATOM | 7868 | OD1 | ASN | A | 528 | 5.990 | 45.973 | 35.798 | 1.00 | 77.45 | O |
| ATOM | 7869 | ND2 | ASN | A | 528 | 7.978 | 44.952 | 35.353 | 1.00 | 77.81 | N |
| ATOM | 7872 | C | ASN | A | 528 | 8.833 | 49.221 | 36.924 | 1.00 | 71.99 | C |
| ATOM | 7873 | O | ASN | A | 528 | 9.871 | 48.815 | 37.396 | 1.00 | 71.09 | O |
| ATOM | 7874 | N | SER | A | 529 | 8.652 | 50.478 | 36.543 | 1.00 | 72.85 | N |
| ATOM | 7876 | CA | SER | A | 529 | 9.608 | 51.521 | 36.862 | 1.00 | 71.65 | C |
| ATOM | 7878 | CB | SER | A | 529 | 9.699 | 51.713 | 38.387 | 1.00 | 72.07 | C |
| ATOM | 7881 | OG | SER | A | 529 | 8.447 | 52.198 | 38.850 | 1.00 | 75.50 | O |
| ATOM | 7883 | C | SER | A | 529 | 10.965 | 51.223 | 36.289 | 1.00 | 68.41 | C |
| ATOM | 7884 | O | SER | A | 529 | 11.971 | 51.654 | 36.838 | 1.00 | 67.15 | O |
| ATOM | 7885 | N | GLU | A | 530 | 11.000 | 50.523 | 35.172 | 1.00 | 66.50 | N |
| ATOM | 7887 | CA | GLU | A | 530 | 12.270 | 50.325 | 34.533 | 1.00 | 65.30 | C |
| ATOM | 7889 | CB | GLU | A | 530 | 12.844 | 48.908 | 34.841 | 1.00 | 64.54 | C |
| ATOM | 7892 | CG | GLU | A | 530 | 12.086 | 47.695 | 34.312 | 1.00 | 67.37 | C |
| ATOM | 7895 | CD | GLU | A | 530 | 12.640 | 46.360 | 34.858 | 1.00 | 71.17 | C |
| ATOM | 7896 | OE1 | GLU | A | 530 | 13.473 | 46.411 | 35.793 | 1.00 | 73.46 | O |
| ATOM | 7897 | OE2 | GLU | A | 530 | 12.263 | 45.242 | 34.358 | 1.00 | 73.29 | O |
| ATOM | 7898 | C | GLU | A | 530 | 12.191 | 50.692 | 33.060 | 1.00 | 64.85 | C |
| ATOM | 7899 | O | GLU | A | 530 | 11.152 | 50.638 | 32.500 | 1.00 | 65.79 | O |
| ATOM | 7900 | N | CYS | A | 531 | 13.302 | 51.128 | 32.477 | 1.00 | 64.45 | N |
| ATOM | 7902 | CA | CYS | A | 531 | 13.370 | 51.702 | 31.133 | 1.00 | 65.38 | C |
| ATOM | 7904 | CB | CYS | A | 531 | 14.157 | 52.986 | 31.109 | 1.00 | 66.14 | C |
| ATOM | 7907 | SG | CYS | A | 531 | 13.296 | 53.919 | 32.284 | 1.00 | 71.12 | S |
| ATOM | 7908 | C | CYS | A | 531 | 14.000 | 50.813 | 30.142 | 1.00 | 64.46 | C |
| ATOM | 7909 | O | CYS | A | 531 | 15.195 | 50.531 | 30.187 | 1.00 | 63.28 | O |
| ATOM | 7910 | N | ILE | A | 532 | 13.178 | 50.491 | 29.167 | 1.00 | 65.37 | N |
| ATOM | 7912 | CA | ILE | A | 532 | 13.468 | 49.509 | 28.187 | 1.00 | 65.17 | C |
| ATOM | 7914 | CB | ILE | A | 532 | 12.381 | 48.517 | 28.302 | 1.00 | 65.55 | C |
| ATOM | 7916 | CG1 | ILE | A | 532 | 12.447 | 47.966 | 29.713 | 1.00 | 66.66 | C |
| ATOM | 7919 | CD1 | ILE | A | 532 | 11.359 | 47.069 | 29.980 | 1.00 | 70.19 | C |
| ATOM | 7923 | CG2 | ILE | A | 532 | 12.541 | 47.427 | 27.274 | 1.00 | 65.55 | C |
| ATOM | 7927 | C | ILE | A | 532 | 13.464 | 50.131 | 26.825 | 1.00 | 65.90 | C |
| ATOM | 7928 | O | ILE | A | 532 | 12.544 | 50.839 | 26.477 | 1.00 | 66.89 | O |
| ATOM | 7929 | N | GLN | A | 533 | 14.500 | 49.835 | 26.049 | 1.00 | 66.08 | N |
| ATOM | 7931 | CA | GLN | A | 533 | 14.651 | 50.416 | 24.711 | 1.00 | 67.62 | C |
| ATOM | 7933 | CB | GLN | A | 533 | 15.969 | 49.974 | 24.060 | 1.00 | 67.56 | C |
| ATOM | 7936 | CG | GLN | A | 533 | 17.233 | 50.681 | 24.519 | 1.00 | 69.80 | C |
| ATOM | 7939 | CD | GLN | A | 533 | 18.451 | 49.923 | 24.040 | 1.00 | 70.83 | C |
| ATOM | 7940 | OE1 | GLN | A | 533 | 18.620 | 49.748 | 22.845 | 1.00 | 72.01 | O |
| ATOM | 7941 | NE2 | GLN | A | 533 | 19.251 | 49.412 | 24.962 | 1.00 | 71.14 | N |
| ATOM | 7944 | C | GLN | A | 533 | 13.558 | 49.968 | 23.770 | 1.00 | 67.44 | C |
| ATOM | 7945 | O | GLN | A | 533 | 13.113 | 48.818 | 23.791 | 1.00 | 65.57 | O |
| ATOM | 7946 | N | CYS | A | 534 | 13.183 | 50.881 | 22.895 | 1.00 | 68.65 | N |
| ATOM | 7948 | CA | CYS | A | 534 | 12.323 | 50.512 | 21.786 | 1.00 | 69.43 | C |
| ATOM | 7950 | CB | CYS | A | 534 | 11.753 | 51.740 | 21.154 | 1.00 | 71.08 | C |
| ATOM | 7953 | SG | CYS | A | 534 | 10.611 | 52.557 | 22.300 | 1.00 | 73.86 | S |
| ATOM | 7954 | C | CYS | A | 534 | 13.126 | 49.751 | 20.777 | 1.00 | 68.25 | C |
| ATOM | 7955 | O | CYS | A | 534 | 14.262 | 50.108 | 20.508 | 1.00 | 68.52 | O |
| ATOM | 7956 | N | HIS | A | 535 | 12.543 | 48.707 | 20.222 | 1.00 | 66.84 | N |
| ATOM | 7958 | CA | HIS | A | 535 | 13.212 | 48.012 | 19.163 | 1.00 | 65.78 | C |
| ATOM | 7960 | CB | HIS | A | 535 | 12.216 | 47.085 | 18.497 | 1.00 | 66.02 | C |
| ATOM | 7963 | CG | HIS | A | 535 | 12.824 | 46.218 | 17.462 | 1.00 | 64.32 | C |
| ATOM | 7964 | ND1 | HIS | A | 535 | 13.031 | 46.638 | 16.175 | 1.00 | 61.92 | N |
| ATOM | 7966 | CE1 | HIS | A | 535 | 13.599 | 45.677 | 15.489 | 1.00 | 64.12 | C |
| ATOM | 7968 | NE2 | HIS | A | 535 | 13.800 | 44.658 | 16.299 | 1.00 | 65.31 | N |
| ATOM | 7970 | CD2 | HIS | A | 535 | 13.337 | 44.980 | 17.543 | 1.00 | 64.19 | C |
| ATOM | 7972 | C | HIS | A | 535 | 13.822 | 49.045 | 18.165 | 1.00 | 66.70 | C |
| ATOM | 7973 | O | HIS | A | 535 | 13.217 | 50.069 | 17.907 | 1.00 | 68.03 | O |
| ATOM | 7974 | N | PRO | A | 536 | 15.028 | 48.800 | 17.637 | 1.00 | 66.72 | N |
| ATOM | 7975 | CA | PRO | A | 536 | 15.804 | 49.881 | 16.971 | 1.00 | 67.56 | C |
| ATOM | 7977 | CB | PRO | A | 536 | 17.231 | 49.296 | 16.821 | 1.00 | 67.39 | C |
| ATOM | 7980 | CG | PRO | A | 536 | 17.085 | 47.783 | 16.953 | 1.00 | 66.07 | C |
| ATOM | 7983 | CD | PRO | A | 536 | 15.762 | 47.502 | 17.657 | 1.00 | 65.17 | C |
| ATOM | 7986 | C | PRO | A | 536 | 15.311 | 50.179 | 15.631 | 1.00 | 68.62 | C |
| ATOM | 7987 | O | PRO | A | 536 | 15.855 | 51.051 | 14.996 | 1.00 | 70.04 | O |
| ATOM | 7988 | N | GLU | A | 537 | 14.367 | 49.374 | 15.170 | 1.00 | 68.61 | N |
| ATOM | 7990 | CA | GLU | A | 537 | 13.650 | 49.638 | 13.937 | 1.00 | 69.99 | C |
| ATOM | 7992 | CB | GLU | A | 537 | 13.211 | 48.324 | 13.277 | 1.00 | 68.51 | C |
| ATOM | 7995 | CG | GLU | A | 537 | 14.325 | 47.611 | 12.610 | 1.00 | 68.09 | C |
| ATOM | 7998 | CD | GLU | A | 537 | 15.045 | 48.492 | 11.626 | 1.00 | 70.66 | C |
| ATOM | 7999 | OE1 | GLU | A | 537 | 14.402 | 49.367 | 11.003 | 1.00 | 74.79 | O |
| ATOM | 8000 | OE2 | GLU | A | 537 | 16.267 | 48.319 | 11.485 | 1.00 | 72.71 | O |
| ATOM | 8001 | C | GLU | A | 537 | 12.418 | 50.521 | 14.139 | 1.00 | 71.72 | C |
| ATOM | 8002 | O | GLU | A | 537 | 11.672 | 50.711 | 13.201 | 1.00 | 74.11 | O |
| ATOM | 8003 | N | CYS | A | 538 | 12.169 | 51.060 | 15.325 | 1.00 | 71.85 | N |
| ATOM | 8005 | CA | CYS | A | 538 | 11.143 | 52.088 | 15.421 | 1.00 | 73.89 | C |
| ATOM | 8007 | CB | CYS | A | 538 | 10.500 | 52.152 | 16.780 | 1.00 | 74.04 | C |
| ATOM | 8010 | SG | CYS | A | 538 | 9.888 | 50.553 | 17.320 | 1.00 | 72.72 | S |
| ATOM | 8011 | C | CYS | A | 538 | 11.770 | 53.404 | 15.135 | 1.00 | 75.65 | C |
| ATOM | 8012 | O | CYS | A | 538 | 12.918 | 53.645 | 15.498 | 1.00 | 74.35 | O |
| ATOM | 8013 | N | LEU | A | 539 | 10.989 | 54.235 | 14.445 | 1.00 | 78.81 | N |
| ATOM | 8015 | CA | LEU | A | 539 | 11.368 | 55.584 | 14.097 | 1.00 | 81.09 | C |
| ATOM | 8017 | CB | LEU | A | 539 | 10.672 | 55.993 | 12.829 | 1.00 | 83.53 | C |
| ATOM | 8020 | CG | LEU | A | 539 | 11.126 | 57.328 | 12.277 | 1.00 | 86.35 | C |
| ATOM | 8022 | CD1 | LEU | A | 539 | 12.585 | 57.287 | 11.828 | 1.00 | 85.10 | C |
| ATOM | 8026 | CD2 | LEU | A | 539 | 10.163 | 57.659 | 11.175 | 1.00 | 88.79 | C |
| ATOM | 8030 | C | LEU | A | 539 | 10.960 | 56.483 | 15.249 | 1.00 | 82.49 | C |
| ATOM | 8031 | O | LEU | A | 539 | 9.773 | 56.549 | 15.611 | 1.00 | 82.07 | O |
| ATOM | 8032 | N | PRO | A | 540 | 11.968 | 57.076 | 15.893 | 1.00 | 83.31 | N |
| ATOM | 8033 | CA | PRO | A | 540 | 11.736 | 58.147 | 16.878 | 1.00 | 84.93 | C |
| ATOM | 8035 | CB | PRO | A | 540 | 13.129 | 58.776 | 17.104 | 1.00 | 85.34 | C |
| ATOM | 8038 | CG | PRO | A | 540 | 14.139 | 57.881 | 16.384 | 1.00 | 84.21 | C |
| ATOM | 8041 | CD | PRO | A | 540 | 13.398 | 56.694 | 15.818 | 1.00 | 81.81 | C |
| ATOM | 8044 | C | PRO | A | 540 | 10.784 | 59.185 | 16.374 | 1.00 | 88.12 | C |
| ATOM | 8045 | O | PRO | A | 540 | 10.908 | 59.595 | 15.238 | 1.00 | 89.43 | O |
| ATOM | 8046 | N | GLN | A | 541 | 9.886 | 59.604 | 17.258 | 1.00 | 89.34 | N |
| ATOM | 8048 | CA | GLN | A | 541 | 8.794 | 60.495 | 16.962 | 1.00 | 92.72 | C |
| ATOM | 8050 | CB | GLN | A | 541 | 7.463 | 59.830 | 17.324 | 1.00 | 92.16 | C |
| ATOM | 8053 | CG | GLN | A | 541 | 7.171 | 58.527 | 16.577 | 1.00 | 89.77 | C |
| ATOM | 8056 | CD | GLN | A | 541 | 6.354 | 58.684 | 15.285 | 1.00 | 92.88 | C |
| ATOM | 8057 | OE1 | GLN | A | 541 | 5.697 | 59.736 | 15.027 | 1.00 | 96.32 | O |
| ATOM | 8058 | NE2 | GLN | A | 541 | 6.385 | 57.629 | 14.462 | 1.00 | 88.98 | N |
| ATOM | 8061 | C | GLN | A | 541 | 8.961 | 61.755 | 17.791 | 1.00 | 95.58 | C |
| ATOM | 8062 | O | GLN | A | 541 | 8.962 | 61.700 | 19.017 | 1.00 | 93.76 | O |
| ATOM | 8063 | N | ALA | A | 542 | 9.043 | 62.898 | 17.103 | 1.00 | 100.12 | N |
| ATOM | 8065 | CA | ALA | A | 542 | 9.171 | 64.199 | 17.758 | 1.00 | 103.21 | C |
| ATOM | 8067 | CB | ALA | A | 542 | 9.082 | 65.306 | 16.720 | 1.00 | 107.95 | C |
| ATOM | 8071 | C | ALA | A | 542 | 8.092 | 64.366 | 18.819 | 1.00 | 104.03 | C |
| ATOM | 8072 | O | ALA | A | 542 | 8.396 | 64.498 | 19.999 | 1.00 | 103.11 | O |
| ATOM | 8073 | N | MET | A | 543 | 6.834 | 64.281 | 18.400 | 1.00 | 105.83 | N |
| ATOM | 8075 | CA | MET | A | 543 | 5.718 | 64.631 | 19.277 | 1.00 | 108.35 | C |
| ATOM | 8077 | CB | MET | A | 543 | 4.430 | 64.701 | 18.477 | 1.00 | 111.35 | C |
| ATOM | 8080 | CG | MET | A | 543 | 4.523 | 65.603 | 17.241 | 1.00 | 116.25 | C |
| ATOM | 8083 | SD | MET | A | 543 | 4.822 | 67.395 | 17.470 | 1.00 | 123.15 | S |
| ATOM | 8084 | CE | MET | A | 543 | 6.486 | 67.662 | 18.473 | 1.00 | 119.09 | C |
| ATOM | 8088 | C | MET | A | 543 | 5.518 | 63.708 | 20.482 | 1.00 | 105.56 | C |
| ATOM | 8089 | O | MET | A | 543 | 5.645 | 64.161 | 21.642 | 1.00 | 105.64 | O |
| ATOM | 8090 | N | ASN | A | 544 | 5.215 | 62.425 | 20.192 | 1.00 | 102.85 | N |
| ATOM | 8092 | CA | ASN | A | 544 | 4.756 | 61.436 | 21.189 | 1.00 | 99.69 | C |
| ATOM | 8094 | CB | ASN | A | 544 | 3.440 | 60.756 | 20.758 | 1.00 | 100.63 | C |
| ATOM | 8097 | CG | ASN | A | 544 | 2.311 | 61.737 | 20.593 | 1.00 | 106.66 | C |
| ATOM | 8098 | OD1 | ASN | A | 544 | 2.108 | 62.599 | 21.452 | 1.00 | 113.63 | O |
| ATOM | 8099 | ND2 | ASN | A | 544 | 1.587 | 61.647 | 19.474 | 1.00 | 107.70 | N |
| ATOM | 8102 | C | ASN | A | 544 | 5.730 | 60.329 | 21.403 | 1.00 | 94.33 | C |
| ATOM | 8103 | O | ASN | A | 544 | 6.730 | 60.230 | 20.713 | 1.00 | 93.08 | O |
| ATOM | 8104 | N | ILE | A | 545 | 5.400 | 59.553 | 22.433 | 1.00 | 91.45 | N |
| ATOM | 8106 | CA | ILE | A | 545 | 5.741 | 58.160 | 22.629 | 1.00 | 86.98 | C |
| ATOM | 8108 | CB | ILE | A | 545 | 4.474 | 57.465 | 23.309 | 1.00 | 86.92 | C |
| ATOM | 8110 | CG1 | ILE | A | 545 | 4.672 | 57.482 | 24.841 | 1.00 | 89.02 | C |
| ATOM | 8113 | CD1 | ILE | A | 545 | 3.522 | 56.919 | 25.781 | 1.00 | 91.04 | C |
| ATOM | 8117 | CG2 | ILE | A | 545 | 4.136 | 56.089 | 22.768 | 1.00 | 82.26 | C |
| ATOM | 8121 | C | ILE | A | 545 | 6.144 | 57.489 | 21.337 | 1.00 | 86.03 | C |
| ATOM | 8122 | O | ILE | A | 545 | 5.452 | 57.604 | 20.316 | 1.00 | 88.94 | O |
| ATOM | 8123 | N | THR | A | 546 | 7.267 | 56.788 | 21.380 | 1.00 | 82.71 | N |
| ATOM | 8125 | CA | THR | A | 546 | 7.828 | 56.125 | 20.215 | 1.00 | 80.76 | C |
| ATOM | 8127 | CB | THR | A | 546 | 9.312 | 56.458 | 20.162 | 1.00 | 79.71 | C |
| ATOM | 8129 | OG1 | THR | A | 546 | 9.431 | 57.779 | 19.651 | 1.00 | 81.40 | O |
| ATOM | 8131 | CG2 | THR | A | 546 | 10.108 | 55.583 | 19.160 | 1.00 | 77.72 | C |
| ATOM | 8135 | C | THR | A | 546 | 7.623 | 54.612 | 20.203 | 1.00 | 78.40 | C |
| ATOM | 8136 | O | THR | A | 546 | 8.110 | 53.971 | 19.274 | 1.00 | 76.95 | O |
| ATOM | 8137 | N | CYS | A | 547 | 6.944 | 54.045 | 21.218 | 1.00 | 77.55 | N |
| ATOM | 8139 | CA | CYS | A | 547 | 6.542 | 52.622 | 21.189 | 1.00 | 76.32 | C |
| ATOM | 8141 | CB | CYS | A | 547 | 7.766 | 51.712 | 21.067 | 1.00 | 73.37 | C |
| ATOM | 8144 | SG | CYS | A | 547 | 8.789 | 51.650 | 22.513 | 1.00 | 73.09 | S |
| ATOM | 8145 | C | CYS | A | 547 | 5.688 | 52.116 | 22.344 | 1.00 | 75.94 | C |
| ATOM | 8146 | O | CYS | A | 547 | 5.642 | 52.743 | 23.343 | 1.00 | 76.38 | O |
| ATOM | 8147 | N | THR | A | 548 | 5.047 | 50.955 | 22.188 | 1.00 | 76.01 | N |
| ATOM | 8149 | CA | THR | A | 548 | 4.280 | 50.292 | 23.254 | 1.00 | 76.89 | C |
| ATOM | 8151 | CB | THR | A | 548 | 3.066 | 49.538 | 22.668 | 1.00 | 78.86 | C |
| ATOM | 8153 | OG1 | THR | A | 548 | 2.220 | 50.450 | 21.967 | 1.00 | 82.65 | O |
| ATOM | 8155 | CG2 | THR | A | 548 | 2.142 | 48.955 | 23.780 | 1.00 | 80.14 | C |
| ATOM | 8159 | C | THR | A | 548 | 5.071 | 49.259 | 24.089 | 1.00 | 74.79 | C |
| ATOM | 8160 | O | THR | A | 548 | 4.572 | 48.822 | 25.099 | 1.00 | 75.56 | O |
| ATOM | 8161 | N | GYL | A | 549 | 6.289 | 48.875 | 23.701 | 1.00 | 72.66 | N |
| ATOM | 8163 | CA | GYL | A | 549 | 6.888 | 47.648 | 24.191 | 1.00 | 70.37 | C |
| ATOM | 8166 | C | GYL | A | 549 | 7.998 | 47.174 | 23.273 | 1.00 | 69.28 | C |
| ATOM | 8167 | O | GYL | A | 549 | 8.481 | 47.923 | 22.394 | 1.00 | 69.27 | O |
| ATOM | 8168 | N | ARG | A | 550 | 8.370 | 45.904 | 23.434 | 1.00 | 68.41 | N |
| ATOM | 8170 | CA | ARG | A | 550 | 9.617 | 45.393 | 22.863 | 1.00 | 66.94 | C |
| ATOM | 8172 | CB | ARG | A | 550 | 10.066 | 44.051 | 23.463 | 1.00 | 66.83 | C |
| ATOM | 8175 | CG | ARG | A | 550 | 9.594 | 43.646 | 24.804 | 1.00 | 67.69 | C |
| ATOM | 8178 | CD | ARG | A | 550 | 10.696 | 43.065 | 25.661 | 1.00 | 67.99 | C |
| ATOM | 8181 | NE | ARG | A | 550 | 10.467 | 43.536 | 27.012 | 1.00 | 68.71 | N |
| ATOM | 8183 | CZ | ARG | A | 550 | 11.350 | 43.538 | 27.946 | 1.00 | 68.02 | C |
| ATOM | 8184 | NH1 | ARG | A | 550 | 12.548 | 43.056 | 27.689 | 1.00 | 71.35 | N |
| ATOM | 8187 | NH2 | ARG | A | 550 | 11.035 | 43.998 | 29.154 | 1.00 | 67.97 | N |
| ATOM | 8190 | C | ARG | A | 550 | 9.593 | 45.083 | 21.401 | 1.00 | 67.18 | C |
| ATOM | 8191 | O | ARG | A | 550 | 10.654 | 45.058 | 20.759 | 1.00 | 65.99 | O |
| ATOM | 8192 | N | GYL | A | 551 | 8.434 | 44.712 | 20.882 | 1.00 | 68.91 | N |
| ATOM | 8194 | CA | GYL | A | 551 | 8.428 | 44.078 | 19.566 | 1.00 | 69.72 | C |
| ATOM | 8197 | C | GYL | A | 551 | 8.984 | 44.980 | 18.462 | 1.00 | 69.41 | C |
| ATOM | 8198 | O | GYL | A | 551 | 9.184 | 46.175 | 18.685 | 1.00 | 69.05 | O |
| ATOM | 8199 | N | PRO | A | 552 | 9.316 | 44.417 | 17.307 | 1.00 | 69.02 | N |
| ATOM | 8200 | CA | PRO | A | 552 | 9.076 | 45.130 | 16.050 | 1.00 | 70.16 | C |
| ATOM | 8202 | CB | PRO | A | 552 | 9.704 | 44.221 | 14.988 | 1.00 | 69.01 | C |
| ATOM | 8205 | CG | PRO | A | 552 | 9.805 | 42.977 | 15.585 | 1.00 | 68.43 | C |
| ATOM | 8208 | CD | PRO | A | 552 | 10.065 | 43.179 | 17.085 | 1.00 | 67.57 | C |
| ATOM | 8211 | C | PRO | A | 552 | 7.584 | 45.371 | 15.794 | 1.00 | 72.55 | C |
| ATOM | 8212 | O | PRO | A | 552 | 7.216 | 46.194 | 14.960 | 1.00 | 74.90 | O |
| ATOM | 8213 | N | ASP | A | 553 | 6.708 | 44.702 | 16.518 | 1.00 | 73.65 | N |
| ATOM | 8215 | CA | ASP | A | 553 | 5.288 | 44.960 | 16.308 | 1.00 | 76.41 | C |
| ATOM | 8217 | CB | ASP | A | 553 | 4.482 | 43.789 | 16.845 | 1.00 | 77.64 | C |
| ATOM | 8220 | CG | ASP | A | 553 | 4.690 | 42.533 | 16.029 | 1.00 | 80.59 | C |
| ATOM | 8221 | OD1 | ASP | A | 553 | 5.877 | 42.294 | 15.647 | 1.00 | 83.78 | O |
| ATOM | 8222 | OD2 | ASP | A | 553 | 3.750 | 41.737 | 15.698 | 1.00 | 84.59 | O |
| ATOM | 8223 | C | ASP | A | 553 | 4.864 | 46.275 | 16.990 | 1.00 | 77.13 | C |
| ATOM | 8224 | O | ASP | A | 553 | 4.011 | 47.012 | 16.498 | 1.00 | 79.38 | O |
| ATOM | 8225 | N | ASN | A | 554 | 5.493 | 46.576 | 18.117 | 1.00 | 75.22 | N |
| ATOM | 8227 | CA | ASN | A | 554 | 5.081 | 47.687 | 18.953 | 1.00 | 75.56 | C |
| ATOM | 8229 | CB | ASN | A | 554 | 5.296 | 47.312 | 20.423 | 1.00 | 75.01 | C |
| ATOM | 8232 | CG | ASN | A | 554 | 4.457 | 46.113 | 20.854 | 1.00 | 76.66 | C |
| ATOM | 8233 | OD1 | ASN | A | 554 | 3.321 | 45.920 | 20.378 | 1.00 | 79.39 | O |
| ATOM | 8234 | ND2 | ASN | A | 554 | 5.013 | 45.299 | 21.759 | 1.00 | 76.10 | N |
| ATOM | 8237 | C | ASN | A | 554 | 5.763 | 49.021 | 18.653 | 1.00 | 74.89 | C |
| ATOM | 8238 | O | ASN | A | 554 | 5.884 | 49.859 | 19.531 | 1.00 | 73.74 | O |
| ATOM | 8239 | N | CYS | A | 555 | 6.191 | 49.225 | 17.414 | 1.00 | 74.76 | N |
| ATOM | 8241 | CA | CYS | A | 555 | 6.585 | 50.553 | 16.971 | 1.00 | 76.15 | C |
| ATOM | 8243 | CB | CYS | A | 555 | 7.378 | 50.459 | 15.691 | 1.00 | 75.87 | C |
| ATOM | 8246 | SG | CYS | A | 555 | 8.888 | 49.525 | 15.840 | 1.00 | 74.07 | S |
| ATOM | 8247 | C | CYS | A | 555 | 5.378 | 51.399 | 16.664 | 1.00 | 79.28 | C |
| ATOM | 8248 | O | CYS | A | 555 | 4.303 | 50.908 | 16.314 | 1.00 | 80.23 | O |
| ATOM | 8249 | N | ILE | A | 556 | 5.549 | 52.690 | 16.757 | 1.00 | 81.45 | N |
| ATOM | 8251 | CA | ILE | A | 556 | 4.588 | 53.558 | 16.076 | 1.00 | 86.21 | C |
| ATOM | 8253 | CB | ILE | A | 556 | 4.217 | 54.775 | 16.994 | 1.00 | 88.59 | C |
| ATOM | 8255 | CG1 | ILE | A | 556 | 3.285 | 54.332 | 18.145 | 1.00 | 88.42 | C |
| ATOM | 8258 | CD1 | ILE | A | 556 | 2.945 | 55.465 | 19.180 | 1.00 | 90.10 | C |
| ATOM | 8262 | CG2 | ILE | A | 556 | 3.656 | 55.923 | 16.146 | 1.00 | 91.71 | C |
| ATOM | 8266 | C | ILE | A | 556 | 5.349 | 54.028 | 14.841 | 1.00 | 86.58 | C |
| ATOM | 8267 | O | ILE | A | 556 | 6.353 | 54.729 | 14.990 | 1.00 | 89.16 | O |
| ATOM | 8268 | N | GLN | A | 557 | 4.994 | 53.618 | 13.653 | 1.00 | 85.39 | N |
| ATOM | 8270 | CA | GLN | A | 557 | 5.882 | 53.937 | 12.530 | 1.00 | 85.32 | C |
| ATOM | 8272 | CB | GLN | A | 557 | 6.184 | 55.456 | 12.453 | 1.00 | 87.88 | C |
| ATOM | 8275 | CG | GLN | A | 557 | 6.910 | 55.832 | 11.136 | 1.00 | 89.41 | C |
| ATOM | 8278 | CD | GLN | A | 557 | 6.557 | 57.164 | 10.557 | 1.00 | 91.93 | C |
| ATOM | 8279 | OE1 | GLN | A | 557 | 6.459 | 58.148 | 11.270 | 1.00 | 95.30 | O |
| ATOM | 8280 | NE2 | GLN | A | 557 | 6.423 | 57.209 | 9.247 | 1.00 | 92.15 | N |
| ATOM | 8283 | C | GLN | A | 557 | 7.229 | 53.165 | 12.420 | 1.00 | 81.67 | C |
| ATOM | 8284 | O | GLN | A | 557 | 8.085 | 53.251 | 13.261 | 1.00 | 80.12 | O |
| ATOM | 8285 | N | CYS | A | 558 | 7.428 | 52.507 | 11.295 | 1.00 | 81.23 | N |
| ATOM | 8287 | CA | CYS | A | 558 | 8.641 | 51.768 | 11.012 | 1.00 | 79.48 | C |
| ATOM | 8289 | CB | CYS | A | 558 | 8.296 | 50.683 | 10.007 | 1.00 | 79.37 | C |
| ATOM | 8292 | SG | CYS | A | 558 | 7.573 | 49.169 | 10.702 | 1.00 | 81.19 | S |
| ATOM | 8293 | C | CYS | A | 558 | 9.759 | 52.580 | 10.388 | 1.00 | 79.80 | C |
| ATOM | 8294 | O | CYS | A | 558 | 9.589 | 53.173 | 9.377 | 1.00 | 81.16 | O |
| ATOM | 8295 | N | ALA | A | 559 | 10.950 | 52.512 | 10.935 | 1.00 | 78.94 | N |
| ATOM | 8297 | CA | ALA | A | 559 | 12.079 | 53.313 | 10.421 | 1.00 | 80.38 | C |
| ATOM | 8299 | CB | ALA | A | 559 | 13.371 | 53.087 | 11.285 | 1.00 | 78.77 | C |
| ATOM | 8303 | C | ALA | A | 559 | 12.432 | 53.046 | 8.989 | 1.00 | 80.70 | C |
| ATOM | 8304 | O | ALA | A | 559 | 12.772 | 53.965 | 8.290 | 1.00 | 83.71 | O |
| ATOM | 8305 | N | HIS | A | 560 | 12.415 | 51.785 | 8.580 | 1.00 | 78.83 | N |
| ATOM | 8307 | CA | HIS | A | 560 | 13.025 | 51.377 | 7.327 | 1.00 | 78.93 | C |
| ATOM | 8309 | CB | HIS | A | 560 | 14.232 | 50.473 | 7.616 | 1.00 | 77.41 | C |
| ATOM | 8312 | CG | HIS | A | 560 | 15.521 | 51.202 | 7.931 | 1.00 | 79.72 | C |
| ATOM | 8313 | ND1 | HIS | A | 560 | 16.068 | 52.180 | 7.112 | 1.00 | 82.70 | N |
| ATOM | 8315 | CE1 | HIS | A | 560 | 17.212 | 52.607 | 7.631 | 1.00 | 84.13 | C |
| ATOM | 8317 | NE2 | HIS | A | 560 | 17.429 | 51.939 | 8.758 | 1.00 | 83.15 | N |
| ATOM | 8319 | CD2 | HIS | A | 560 | 16.395 | 51.050 | 8.962 | 1.00 | 79.11 | C |
| ATOM | 8321 | C | HIS | A | 560 | 11.932 | 50.680 | 6.503 | 1.00 | 78.66 | C |
| ATOM | 8322 | O | HIS | A | 560 | 11.189 | 51.365 | 5.825 | 1.00 | 81.48 | O |
| ATOM | 8323 | N | TYR | A | 561 | 11.771 | 49.354 | 6.575 | 1.00 | 76.45 | N |
| ATOM | 8325 | CA | TYR | A | 561 | 10.666 | 48.684 | 5.844 | 1.00 | 76.16 | C |
| ATOM | 8327 | CB | TYR | A | 561 | 11.162 | 47.651 | 4.814 | 1.00 | 75.26 | C |
| ATOM | 8330 | CG | TYR | A | 561 | 12.188 | 48.263 | 3.937 | 1.00 | 76.43 | C |
| ATOM | 8331 | CD1 | TYR | A | 561 | 13.544 | 48.158 | 4.251 | 1.00 | 76.49 | C |
| ATOM | 8333 | CE1 | TYR | A | 561 | 14.514 | 48.770 | 3.507 | 1.00 | 77.50 | C |
| ATOM | 8335 | CZ | TYR | A | 561 | 14.133 | 49.520 | 2.438 | 1.00 | 81.83 | C |
| ATOM | 8336 | OH | TYR | A | 561 | 15.079 | 50.155 | 1.675 | 1.00 | 86.33 | O |
| ATOM | 8338 | CE2 | TYR | A | 561 | 12.789 | 49.645 | 2.097 | 1.00 | 82.34 | C |
| ATOM | 8340 | CD2 | TYR | A | 561 | 11.824 | 49.028 | 2.861 | 1.00 | 79.04 | C |
| ATOM | 8342 | C | TYR | A | 561 | 9.733 | 48.040 | 6.820 | 1.00 | 74.68 | C |
| ATOM | 8343 | O | TYR | A | 561 | 10.052 | 47.969 | 8.013 | 1.00 | 72.79 | O |
| ATOM | 8344 | N | ILE | A | 562 | 8.576 | 47.624 | 6.285 | 1.00 | 75.37 | N |
| ATOM | 8346 | CA | ILE | A | 562 | 7.533 | 46.899 | 7.014 | 1.00 | 75.42 | C |
| ATOM | 8348 | CB | ILE | A | 562 | 6.207 | 47.710 | 7.246 | 1.00 | 77.81 | C |
| ATOM | 8350 | CG1 | ILE | A | 562 | 6.473 | 49.235 | 7.482 | 1.00 | 81.60 | C |
| ATOM | 8353 | CD1 | ILE | A | 562 | 5.649 | 50.324 | 6.529 | 1.00 | 83.69 | C |
| ATOM | 8357 | CG2 | ILE | A | 562 | 5.419 | 47.117 | 8.432 | 1.00 | 75.53 | C |
| ATOM | 8361 | C | ILE | A | 562 | 7.189 | 45.624 | 6.263 | 1.00 | 75.08 | C |
| ATOM | 8362 | O | ILE | A | 562 | 6.904 | 45.638 | 5.077 | 1.00 | 75.61 | O |
| ATOM | 8363 | N | ASP | A | 563 | 7.225 | 44.529 | 7.012 | 1.00 | 73.98 | N |
| ATOM | 8365 | CA | ASP | A | 563 | 6.927 | 43.193 | 6.553 | 1.00 | 73.94 | C |
| ATOM | 8367 | CB | ASP | A | 563 | 8.170 | 42.341 | 6.827 | 1.00 | 72.30 | C |
| ATOM | 8370 | CG | ASP | A | 563 | 8.107 | 40.945 | 6.226 | 1.00 | 72.50 | C |
| ATOM | 8371 | OD1 | ASP | A | 563 | 7.372 | 40.727 | 5.229 | 1.00 | 70.35 | O |
| ATOM | 8372 | OD2 | ASP | A | 563 | 8.822 | 40.012 | 6.712 | 1.00 | 72.64 | O |
| ATOM | 8373 | C | ASP | A | 563 | 5.760 | 42.742 | 7.401 | 1.00 | 74.39 | C |
| ATOM | 8374 | O | ASP | A | 563 | 5.902 | 42.627 | 8.612 | 1.00 | 73.65 | O |
| ATOM | 8375 | N | GLY | A | 564 | 4.600 | 42.522 | 6.796 | 1.00 | 75.74 | N |
| ATOM | 8377 | CA | GLY | A | 564 | 3.414 | 42.251 | 7.590 | 1.00 | 77.02 | C |
| ATOM | 8380 | C | GLY | A | 564 | 3.269 | 43.192 | 8.781 | 1.00 | 76.38 | C |
| ATOM | 8381 | O | GLY | A | 564 | 3.406 | 44.373 | 8.626 | 1.00 | 75.98 | O |
| ATOM | 8382 | N | PRO | A | 565 | 2.992 | 42.679 | 9.966 | 1.00 | 76.69 | N |
| ATOM | 8383 | CA | PRO | A | 565 | 2.937 | 43.529 | 11.162 | 1.00 | 77.09 | C |
| ATOM | 8385 | CB | PRO | A | 565 | 2.388 | 42.599 | 12.264 | 1.00 | 77.30 | C |
| ATOM | 8388 | CG | PRO | A | 565 | 2.710 | 41.225 | 11.789 | 1.00 | 77.15 | C |
| ATOM | 8391 | CD | PRO | A | 565 | 2.673 | 41.277 | 10.276 | 1.00 | 77.40 | C |
| ATOM | 8394 | C | PRO | A | 565 | 4.264 | 44.083 | 11.612 | 1.00 | 75.13 | C |
| ATOM | 8395 | O | PRO | A | 565 | 4.210 | 45.061 | 12.315 | 1.00 | 76.23 | O |
| ATOM | 8396 | N | HIS | A | 566 | 5.389 | 43.477 | 11.245 | 1.00 | 73.76 | N |
| ATOM | 8398 | CA | HIS | A | 566 | 6.707 | 43.775 | 11.819 | 1.00 | 71.93 | C |
| ATOM | 8400 | CB | HIS | A | 566 | 7.618 | 42.556 | 11.660 | 1.00 | 70.47 | C |
| ATOM | 8403 | CG | HIS | A | 566 | 7.029 | 41.249 | 12.095 | 1.00 | 70.87 | C |
| ATOM | 8404 | ND1 | HIS | A | 566 | 7.119 | 40.789 | 13.387 | 1.00 | 71.81 | N |
| ATOM | 8406 | CE1 | HIS | A | 566 | 6.566 | 39.590 | 13.461 | 1.00 | 74.18 | C |
| ATOM | 8408 | NE2 | HIS | A | 566 | 6.133 | 39.251 | 12.260 | 1.00 | 73.76 | N |
| ATOM | 8410 | CD2 | HIS | A | 566 | 6.430 | 40.264 | 11.386 | 1.00 | 72.64 | C |
| ATOM | 8412 | C | HIS | A | 566 | 7.442 | 44.879 | 11.078 | 1.00 | 72.22 | C |
| ATOM | 8413 | O | HIS | A | 566 | 7.337 | 44.943 | 9.859 | 1.00 | 74.16 | O |
| ATOM | 8414 | N | CYS | A | 567 | 8.246 | 45.687 | 11.769 | 1.00 | 71.03 | N |
| ATOM | 8416 | CA | CYS | A | 567 | 9.234 | 46.549 | 11.105 | 1.00 | 70.86 | C |
| ATOM | 8418 | CB | CYS | A | 567 | 9.604 | 47.726 | 11.972 | 1.00 | 71.52 | C |
| ATOM | 8421 | SG | CYS | A | 567 | 8.241 | 48.666 | 12.577 | 1.00 | 73.87 | S |
| ATOM | 8422 | C | CYS | A | 567 | 10.530 | 45.832 | 10.962 | 1.00 | 69.04 | C |
| ATOM | 8423 | O | CYS | A | 567 | 11.035 | 45.332 | 11.937 | 1.00 | 68.08 | O |
| ATOM | 8424 | N | VAL | A | 568 | 11.128 | 45.862 | 9.788 | 1.00 | 69.34 | N |
| ATOM | 8426 | CA | VAL | A | 568 | 12.431 | 45.284 | 9.614 | 1.00 | 68.53 | C |
| ATOM | 8428 | CB | VAL | A | 568 | 12.355 | 44.035 | 8.788 | 1.00 | 69.32 | C |
| ATOM | 8430 | CG1 | VAL | A | 568 | 13.574 | 42.972 | 9.226 | 1.00 | 69.18 | C |
| ATOM | 8434 | CG2 | VAL | A | 568 | 10.953 | 43.396 | 8.907 | 1.00 | 70.17 | C |
| ATOM | 8438 | C | VAL | A | 568 | 13.381 | 46.205 | 8.951 | 1.00 | 68.70 | C |
| ATOM | 8439 | O | VAL | A | 568 | 12.953 | 47.118 | 8.287 | 1.00 | 71.27 | O |
| ATOM | 8440 | N | LYS | A | 569 | 14.673 | 45.970 | 9.158 | 1.00 | 67.78 | N |
| ATOM | 8442 | CA | LYS | A | 569 | 15.725 | 46.687 | 8.455 | 1.00 | 69.12 | C |
| ATOM | 8444 | CB | LYS | A | 569 | 17.100 | 46.328 | 9.026 | 1.00 | 68.55 | C |
| ATOM | 8447 | CG | LYS | A | 569 | 18.246 | 47.213 | 8.440 | 1.00 | 72.13 | C |
| ATOM | 8450 | CD | LYS | A | 569 | 19.587 | 47.155 | 9.229 | 1.00 | 73.34 | C |
| ATOM | 8453 | CE | LYS | A | 569 | 20.258 | 48.558 | 9.307 | 1.00 | 75.82 | C |
| ATOM | 8456 | NZ | LYS | A | 569 | 21.618 | 48.573 | 9.964 | 1.00 | 77.31 | N |
| ATOM | 8460 | C | LYS | A | 569 | 15.732 | 46.425 | 6.945 | 1.00 | 70.04 | C |
| ATOM | 8461 | O | LYS | A | 569 | 16.006 | 47.328 | 6.144 | 1.00 | 71.03 | O |
| ATOM | 8462 | N | THR | A | 570 | 15.498 | 45.170 | 6.573 | 1.00 | 69.61 | N |
| ATOM | 8464 | CA | THR | A | 570 | 15.401 | 44.774 | 5.179 | 1.00 | 70.80 | C |
| ATOM | 8466 | CB | THR | A | 570 | 16.551 | 43.874 | 4.758 | 1.00 | 70.64 | C |
| ATOM | 8468 | OG1 | THR | A | 570 | 17.678 | 44.132 | 5.574 | 1.00 | 69.69 | O |
| ATOM | 8470 | CG2 | THR | A | 570 | 17.028 | 44.225 | 3.290 | 1.00 | 73.62 | C |
| ATOM | 8474 | C | THR | A | 570 | 14.147 | 43.992 | 4.941 | 1.00 | 71.10 | C |
| ATOM | 8475 | O | THR | A | 570 | 13.566 | 43.417 | 5.860 | 1.00 | 70.34 | O |
| ATOM | 8476 | N | CYS | A | 571 | 13.727 | 43.956 | 3.684 | 1.00 | 72.17 | N |
| ATOM | 8478 | CA | CYS | A | 571 | 12.740 | 42.992 | 3.308 | 1.00 | 72.52 | C |
| ATOM | 8480 | CB | CYS | A | 571 | 12.089 | 43.404 | 2.008 | 1.00 | 74.33 | C |
| ATOM | 8483 | SG | CYS | A | 571 | 10.724 | 44.604 | 2.195 | 1.00 | 78.47 | S |
| ATOM | 8484 | C | CYS | A | 571 | 13.468 | 41.631 | 3.216 | 1.00 | 71.56 | C |
| ATOM | 8485 | O | CYS | A | 571 | 14.624 | 41.563 | 2.831 | 1.00 | 71.33 | O |
| ATOM | 8486 | N | PRO | A | 572 | 12.807 | 40.570 | 3.634 | 1.00 | 71.21 | N |
| ATOM | 8487 | CA | PRO | A | 572 | 13.365 | 39.208 | 3.601 | 1.00 | 70.85 | C |
| ATOM | 8489 | CB | PRO | A | 572 | 12.172 | 38.339 | 3.923 | 1.00 | 71.07 | C |
| ATOM | 8492 | CG | PRO | A | 572 | 11.307 | 39.225 | 4.741 | 1.00 | 72.16 | C |
| ATOM | 8495 | CD | PRO | A | 572 | 11.470 | 40.617 | 4.231 | 1.00 | 71.58 | C |
| ATOM | 8498 | C | PRO | A | 572 | 13.861 | 38.806 | 2.253 | 1.00 | 71.37 | C |
| ATOM | 8499 | O | PRO | A | 572 | 13.118 | 38.873 | 1.309 | 1.00 | 71.71 | O |
| ATOM | 8500 | N | ALA | A | 573 | 15.091 | 38.332 | 2.192 | 1.00 | 71.59 | N |
| ATOM | 8502 | CA | ALA | A | 573 | 15.743 | 37.986 | 0.933 | 1.00 | 72.60 | C |
| ATOM | 8504 | CB | ALA | A | 573 | 16.655 | 39.103 | 0.532 | 1.00 | 72.49 | C |
| ATOM | 8508 | C | ALA | A | 573 | 16.537 | 36.667 | 1.074 | 1.00 | 72.93 | C |
| ATOM | 8509 | O | ALA | A | 573 | 17.586 | 36.608 | 1.717 | 1.00 | 73.24 | O |
| ATOM | 8510 | N | GLY | A | 574 | 16.044 | 35.603 | 0.472 | 1.00 | 73.55 | N |
| ATOM | 8512 | CA | GLY | A | 574 | 16.781 | 34.355 | 0.481 | 1.00 | 75.15 | C |
| ATOM | 8515 | C | GLY | A | 574 | 16.810 | 33.712 | 1.853 | 1.00 | 74.56 | C |
| ATOM | 8516 | O | GLY | A | 574 | 17.826 | 33.607 | 2.501 | 1.00 | 74.55 | O |
| ATOM | 8517 | N | VAL | A | 575 | 15.638 | 33.301 | 2.271 | 1.00 | 74.53 | N |
| ATOM | 8519 | CA | VAL | A | 575 | 15.376 | 32.861 | 3.612 | 1.00 | 74.16 | C |
| ATOM | 8521 | CB | VAL | A | 575 | 14.468 | 33.930 | 4.276 | 1.00 | 72.51 | C |
| ATOM | 8523 | CG1 | VAL | A | 575 | 13.634 | 33.376 | 5.319 | 1.00 | 73.47 | C |
| ATOM | 8527 | CG2 | VAL | A | 575 | 15.322 | 34.978 | 4.860 | 1.00 | 70.07 | C |
| ATOM | 8531 | C | VAL | A | 575 | 14.734 | 31.494 | 3.412 | 1.00 | 75.88 | C |
| ATOM | 8532 | O | VAL | A | 575 | 14.015 | 31.284 | 2.455 | 1.00 | 76.15 | O |
| ATOM | 8533 | N | MET | A | 576 | 15.001 | 30.517 | 4.243 | 1.00 | 77.05 | N |
| ATOM | 8535 | CA | MET | A | 576 | 14.451 | 29.252 | 3.827 | 1.00 | 79.91 | C |
| ATOM | 8537 | CB | MET | A | 576 | 15.111 | 28.104 | 4.533 | 1.00 | 81.42 | C |
| ATOM | 8540 | CG | MET | A | 576 | 14.645 | 26.801 | 3.936 | 1.00 | 85.73 | C |
| ATOM | 8543 | SD | MET | A | 576 | 14.960 | 26.651 | 2.151 | 1.00 | 89.21 | S |
| ATOM | 8544 | CE | MET | A | 576 | 16.879 | 26.184 | 2.371 | 1.00 | 90.57 | C |
| ATOM | 8548 | C | MET | A | 576 | 12.921 | 29.212 | 3.989 | 1.00 | 80.55 | C |
| ATOM | 8549 | O | MET | A | 576 | 12.415 | 29.721 | 4.978 | 1.00 | 79.42 | O |
| ATOM | 8550 | N | GLY | A | 577 | 12.218 | 28.602 | 3.012 | 1.00 | 82.68 | N |
| ATOM | 8552 | CA | GLY | A | 577 | 10.765 | 28.366 | 3.050 | 1.00 | 84.07 | C |
| ATOM | 8555 | C | GLY | A | 577 | 10.277 | 26.939 | 2.725 | 1.00 | 87.30 | C |
| ATOM | 8556 | O | GLY | A | 577 | 11.075 | 25.988 | 2.639 | 1.00 | 88.11 | O |
| ATOM | 8557 | N | GLU | A | 578 | 8.948 | 26.807 | 2.560 | 1.00 | 89.19 | N |
| ATOM | 8559 | CA | GLU | A | 578 | 8.275 | 25.574 | 2.078 | 1.00 | 92.83 | C |
| ATOM | 8561 | CB | GLU | A | 578 | 6.821 | 25.929 | 1.572 | 1.00 | 93.63 | C |
| ATOM | 8564 | CG | GLU | A | 578 | 5.920 | 24.741 | 1.086 | 1.00 | 98.88 | C |
| ATOM | 8567 | CD | GLU | A | 578 | 5.383 | 24.801 | −0.403 | 1.00 | 100.15 | C |
| ATOM | 8568 | OE1 | GLU | A | 578 | 5.169 | 25.906 | −0.965 | 1.00 | 100.10 | O |
| ATOM | 8569 | OE2 | GLU | A | 578 | 5.120 | 23.730 | −1.024 | 1.00 | 100.27 | O |
| ATOM | 8570 | C | GLU | A | 578 | 9.100 | 24.860 | 0.942 | 1.00 | 94.09 | C |
| ATOM | 8571 | O | GLU | A | 578 | 9.703 | 25.548 | 0.100 | 1.00 | 92.98 | O |
| ATOM | 8572 | N | ASN | A | 579 | 9.092 | 23.509 | 0.905 | 1.00 | 96.70 | N |
| ATOM | 8574 | CA | ASN | A | 579 | 9.654 | 22.694 | −0.210 | 1.00 | 97.87 | C |
| ATOM | 8576 | CB | ASN | A | 579 | 8.673 | 22.664 | −1.431 | 1.00 | 98.97 | C |
| ATOM | 8579 | CG | ASN | A | 579 | 8.787 | 21.320 | −2.343 | 1.00 | 103.58 | C |
| ATOM | 8580 | OD1 | ASN | A | 579 | 9.375 | 20.292 | −1.934 | 1.00 | 107.20 | O |
| ATOM | 8581 | ND2 | ASN | A | 579 | 8.176 | 21.367 | −3.567 | 1.00 | 102.91 | N |
| ATOM | 8584 | C | ASN | A | 579 | 11.088 | 23.139 | −0.599 | 1.00 | 95.88 | C |
| ATOM | 8585 | O | ASN | A | 579 | 11.436 | 23.228 | −1.761 | 1.00 | 95.66 | O |
| ATOM | 8586 | N | ASN | A | 580 | 11.921 | 23.436 | 0.392 | 1.00 | 94.91 | N |
| ATOM | 8588 | CA | ASN | A | 580 | 13.261 | 23.999 | 0.150 | 1.00 | 93.61 | C |
| ATOM | 8590 | CB | ASN | A | 580 | 14.272 | 22.905 | −0.271 | 1.00 | 96.16 | C |
| ATOM | 8593 | CG | ASN | A | 580 | 15.723 | 23.230 | 0.188 | 1.00 | 96.15 | C |
| ATOM | 8594 | OD1 | ASN | A | 580 | 16.025 | 24.371 | 0.471 | 1.00 | 97.19 | O |
| ATOM | 8595 | ND2 | ASN | A | 580 | 16.599 | 22.237 | 0.248 | 1.00 | 96.25 | N |
| ATOM | 8598 | C | ASN | A | 580 | 13.325 | 25.157 | −0.873 | 1.00 | 91.65 | C |
| ATOM | 8599 | O | ASN | A | 580 | 14.368 | 25.364 | −1.497 | 1.00 | 91.22 | O |
| ATOM | 8600 | N | THR | A | 581 | 12.220 | 25.893 | −1.049 | 1.00 | 90.43 | N |
| ATOM | 8602 | CA | THR | A | 581 | 12.204 | 27.090 | −1.901 | 1.00 | 88.39 | C |
| ATOM | 8604 | CB | THR | A | 581 | 10.888 | 27.209 | −2.798 | 1.00 | 89.27 | C |
| ATOM | 8606 | OG1 | THR | A | 581 | 9.764 | 27.589 | −1.994 | 1.00 | 89.04 | O |
| ATOM | 8608 | CG2 | THR | A | 581 | 10.466 | 25.857 | −3.437 | 1.00 | 92.43 | C |
| ATOM | 8612 | C | THR | A | 581 | 12.406 | 28.367 | −1.034 | 1.00 | 84.96 | C |
| ATOM | 8613 | O | THR | A | 581 | 11.544 | 28.728 | −0.221 | 1.00 | 83.83 | O |
| ATOM | 8614 | N | LEU | A | 582 | 13.563 | 29.011 | −1.223 | 1.00 | 82.72 | N |
| ATOM | 8616 | CA | LEU | A | 582 | 13.796 | 30.357 | −0.742 | 1.00 | 79.94 | C |
| ATOM | 8618 | CB | LEU | A | 582 | 15.097 | 30.931 | −1.288 | 1.00 | 79.24 | C |
| ATOM | 8621 | CG | LEU | A | 582 | 16.381 | 30.246 | −0.893 | 1.00 | 80.18 | C |
| ATOM | 8623 | CD1 | LEU | A | 582 | 17.569 | 31.154 | −1.231 | 1.00 | 79.77 | C |
| ATOM | 8627 | CD2 | LEU | A | 582 | 16.366 | 29.908 | 0.573 | 1.00 | 80.16 | C |
| ATOM | 8631 | C | LEU | A | 582 | 12.669 | 31.327 | −1.083 | 1.00 | 78.75 | C |
| ATOM | 8632 | O | LEU | A | 582 | 11.920 | 31.175 | −2.062 | 1.00 | 78.56 | O |
| ATOM | 8633 | N | VAL | A | 583 | 12.627 | 32.351 | −0.238 | 1.00 | 77.04 | N |
| ATOM | 8635 | CA | VAL | A | 583 | 11.565 | 33.320 | −0.172 | 1.00 | 76.73 | C |
| ATOM | 8637 | CB | VAL | A | 583 | 10.838 | 33.269 | 1.201 | 1.00 | 76.17 | C |
| ATOM | 8639 | CG1 | VAL | A | 583 | 9.880 | 34.449 | 1.387 | 1.00 | 75.25 | C |
| ATOM | 8643 | CG2 | VAL | A | 583 | 10.076 | 31.987 | 1.302 | 1.00 | 78.05 | C |
| ATOM | 8647 | C | VAL | A | 583 | 12.202 | 34.664 | −0.367 | 1.00 | 75.52 | C |
| ATOM | 8648 | O | VAL | A | 583 | 13.122 | 35.011 | 0.368 | 1.00 | 74.49 | O |
| ATOM | 8649 | N | TRP | A | 584 | 11.705 | 35.404 | −1.363 | 1.00 | 75.92 | N |
| ATOM | 8651 | CA | TRP | A | 584 | 12.152 | 36.769 | −1.631 | 1.00 | 74.96 | C |
| ATOM | 8653 | CB | TRP | A | 584 | 12.784 | 36.822 | −3.004 | 1.00 | 75.84 | C |
| ATOM | 8656 | CG | TRP | A | 584 | 13.929 | 35.915 | −3.112 | 1.00 | 75.22 | C |
| ATOM | 8657 | CD1 | TRP | A | 584 | 13.892 | 34.622 | −3.468 | 1.00 | 73.95 | C |
| ATOM | 8659 | NE1 | TRP | A | 584 | 15.156 | 34.097 | −3.430 | 1.00 | 72.84 | N |
| ATOM | 8661 | CE2 | TRP | A | 584 | 16.035 | 35.062 | −3.034 | 1.00 | 72.12 | C |
| ATOM | 8662 | CD2 | TRP | A | 584 | 15.298 | 36.220 | −2.810 | 1.00 | 73.54 | C |
| ATOM | 8663 | CE3 | TRP | A | 584 | 15.972 | 37.367 | −2.379 | 1.00 | 73.41 | C |
| ATOM | 8665 | CZ3 | TRP | A | 584 | 17.340 | 37.308 | −2.209 | 1.00 | 74.46 | C |
| ATOM | 8667 | CH2 | TRP | A | 584 | 18.035 | 36.127 | −2.434 | 1.00 | 75.07 | C |
| ATOM | 8669 | CZ2 | TRP | A | 584 | 17.397 | 35.002 | −2.850 | 1.00 | 73.68 | C |
| ATOM | 8671 | C | TRP | A | 584 | 11.015 | 37.767 | −1.589 | 1.00 | 74.89 | C |
| ATOM | 8672 | O | TRP | A | 584 | 9.889 | 37.450 | −1.907 | 1.00 | 75.76 | O |
| ATOM | 8673 | N | LYS | A | 585 | 11.332 | 38.992 | −1.213 | 1.00 | 74.03 | N |
| ATOM | 8675 | CA | LYS | A | 585 | 10.341 | 40.037 | −1.097 | 1.00 | 73.90 | C |
| ATOM | 8677 | CB | LYS | A | 585 | 9.857 | 40.189 | 0.369 | 1.00 | 72.96 | C |
| ATOM | 8680 | CG | LYS | A | 585 | 8.762 | 39.160 | 0.785 | 1.00 | 72.93 | C |
| ATOM | 8683 | CD | LYS | A | 585 | 7.912 | 39.538 | 2.029 | 1.00 | 71.79 | C |
| ATOM | 8686 | CE | LYS | A | 585 | 6.856 | 38.458 | 2.403 | 1.00 | 72.28 | C |
| ATOM | 8689 | NZ | LYS | A | 585 | 6.373 | 38.409 | 3.829 | 1.00 | 71.42 | N |
| ATOM | 8693 | C | LYS | A | 585 | 10.947 | 41.305 | −1.619 | 1.00 | 73.86 | C |
| ATOM | 8694 | O | LYS | A | 585 | 12.140 | 41.474 | −1.545 | 1.00 | 71.84 | O |
| ATOM | 8695 | N | TYR | A | 586 | 10.104 | 42.174 | −2.184 | 1.00 | 75.89 | N |
| ATOM | 8697 | CA | TYR | A | 586 | 10.490 | 43.531 | −2.525 | 1.00 | 77.16 | C |
| ATOM | 8699 | CB | TYR | A | 586 | 10.230 | 43.846 | −3.986 | 1.00 | 79.32 | C |
| ATOM | 8702 | CG | TYR | A | 586 | 8.763 | 43.925 | −4.384 | 1.00 | 81.59 | C |
| ATOM | 8703 | CD1 | TYR | A | 586 | 8.213 | 45.101 | −4.860 | 1.00 | 82.49 | C |
| ATOM | 8705 | CE1 | TYR | A | 586 | 6.859 | 45.174 | −5.239 | 1.00 | 86.87 | C |
| ATOM | 8707 | CZ | TYR | A | 586 | 6.024 | 44.052 | −5.144 | 1.00 | 87.16 | C |
| ATOM | 8708 | OH | TYR | A | 586 | 4.674 | 44.110 | −5.515 | 1.00 | 87.71 | O |
| ATOM | 8710 | CE2 | TYR | A | 586 | 6.560 | 42.871 | −4.672 | 1.00 | 85.36 | C |
| ATOM | 8712 | CD2 | TYR | A | 586 | 7.928 | 42.812 | −4.287 | 1.00 | 82.83 | C |
| ATOM | 8714 | C | TYR | A | 586 | 9.748 | 44.512 | −1.639 | 1.00 | 77.86 | C |
| ATOM | 8715 | O | TYR | A | 586 | 8.764 | 44.169 | −0.970 | 1.00 | 76.82 | O |
| ATOM | 8716 | N | ALA | A | 587 | 10.264 | 45.735 | −1.642 | 1.00 | 79.52 | N |
| ATOM | 8718 | CA | ALA | A | 587 | 9.624 | 46.881 | −1.005 | 1.00 | 81.22 | C |
| ATOM | 8720 | CB | ALA | A | 587 | 10.664 | 47.718 | −0.259 | 1.00 | 81.30 | C |
| ATOM | 8724 | C | ALA | A | 587 | 8.942 | 47.760 | −2.040 | 1.00 | 83.87 | C |
| ATOM | 8725 | O | ALA | A | 587 | 9.587 | 48.225 | −2.966 | 1.00 | 85.06 | O |
| ATOM | 8726 | N | ASP | A | 588 | 7.646 | 48.003 | −1.873 | 1.00 | 85.42 | N |
| ATOM | 8728 | CA | ASP | A | 588 | 6.956 | 49.034 | −2.638 | 1.00 | 87.97 | C |
| ATOM | 8730 | CB | ASP | A | 588 | 5.453 | 48.660 | −2.823 | 1.00 | 89.10 | C |
| ATOM | 8733 | CG | ASP | A | 588 | 4.586 | 48.846 | −1.562 | 1.00 | 88.49 | C |
| ATOM | 8734 | OD1 | ASP | A | 588 | 4.972 | 49.551 | −0.614 | 1.00 | 88.44 | O |
| ATOM | 8735 | OD2 | ASP | A | 588 | 3.453 | 48.328 | −1.455 | 1.00 | 88.07 | O |
| ATOM | 8736 | C | ASP | A | 588 | 7.209 | 50.472 | −2.088 | 1.00 | 89.91 | C |
| ATOM | 8737 | O | ASP | A | 588 | 7.973 | 50.687 | −1.122 | 1.00 | 88.35 | O |
| ATOM | 8738 | N | ALA | A | 589 | 6.571 | 51.439 | −2.751 | 1.00 | 93.88 | N |
| ATOM | 8740 | CA | ALA | A | 589 | 6.641 | 52.881 | −2.458 | 1.00 | 96.73 | C |
| ATOM | 8742 | CB | ALA | A | 589 | 5.728 | 53.626 | −3.421 | 1.00 | 100.11 | C |
| ATOM | 8746 | C | ALA | A | 589 | 6.276 | 53.241 | −1.005 | 1.00 | 96.86 | C |
| ATOM | 8747 | O | ALA | A | 589 | 6.805 | 54.223 | −0.437 | 1.00 | 98.22 | O |
| ATOM | 8748 | N | GLY | A | 590 | 5.363 | 52.441 | −0.433 | 1.00 | 95.88 | N |
| ATOM | 8750 | CA | GLY | A | 590 | 4.993 | 52.493 | 0.975 | 1.00 | 94.90 | C |
| ATOM | 8753 | C | GLY | A | 590 | 6.013 | 51.865 | 1.906 | 1.00 | 91.58 | C |
| ATOM | 8754 | O | GLY | A | 590 | 5.808 | 51.912 | 3.111 | 1.00 | 90.28 | O |
| ATOM | 8755 | N | HIS | A | 591 | 7.114 | 51.337 | 1.348 | 1.00 | 90.22 | N |
| ATOM | 8757 | CA | HIS | A | 591 | 8.120 | 50.545 | 2.075 | 1.00 | 87.77 | C |
| ATOM | 8759 | CB | HIS | A | 591 | 8.959 | 51.439 | 3.071 | 1.00 | 88.45 | C |
| ATOM | 8762 | CG | HIS | A | 591 | 9.793 | 52.510 | 2.391 | 1.00 | 95.05 | C |
| ATOM | 8763 | ND1 | HIS | A | 591 | 9.493 | 53.860 | 2.461 | 1.00 | 103.48 | N |
| ATOM | 8765 | CE1 | HIS | A | 591 | 10.363 | 54.550 | 1.732 | 1.00 | 106.24 | C |
| ATOM | 8767 | NE2 | HIS | A | 591 | 11.225 | 53.702 | 1.194 | 1.00 | 104.71 | N |
| ATOM | 8769 | CD2 | HIS | A | 591 | 10.895 | 52.423 | 1.597 | 1.00 | 98.92 | C |
| ATOM | 8771 | C | HIS | A | 591 | 7.502 | 49.249 | 2.733 | 1.00 | 84.53 | C |
| ATOM | 8772 | O | HIS | A | 591 | 7.962 | 48.785 | 3.794 | 1.00 | 82.29 | O |
| ATOM | 8773 | N | VAL | A | 592 | 6.486 | 48.675 | 2.075 | 1.00 | 83.76 | N |
| ATOM | 8775 | CA | VAL | A | 592 | 5.796 | 47.466 | 2.544 | 1.00 | 81.76 | C |
| ATOM | 8777 | CB | VAL | A | 592 | 4.215 | 47.621 | 2.498 | 1.00 | 83.71 | C |
| ATOM | 8779 | CG1 | VAL | A | 592 | 3.457 | 46.343 | 2.905 | 1.00 | 81.70 | C |
| ATOM | 8783 | CG2 | VAL | A | 592 | 3.769 | 48.747 | 3.396 | 1.00 | 85.59 | C |
| ATOM | 8787 | C | VAL | A | 592 | 6.300 | 46.291 | 1.683 | 1.00 | 80.26 | C |
| ATOM | 8788 | O | VAL | A | 592 | 6.623 | 46.478 | 0.512 | 1.00 | 80.92 | O |
| ATOM | 8789 | N | CYS | A | 593 | 6.362 | 45.087 | 2.259 | 1.00 | 77.78 | N |
| ATOM | 8791 | CA | CYS | A | 593 | 7.075 | 43.998 | 1.639 | 1.00 | 75.78 | C |
| ATOM | 8793 | CB | CYS | A | 593 | 7.847 | 43.219 | 2.689 | 1.00 | 74.24 | C |
| ATOM | 8796 | SG | CYS | A | 593 | 9.221 | 44.051 | 3.477 | 1.00 | 73.14 | S |
| ATOM | 8797 | C | CYS | A | 593 | 6.122 | 43.052 | 1.000 | 1.00 | 76.03 | C |
| ATOM | 8798 | O | CYS | A | 593 | 5.141 | 42.647 | 1.620 | 1.00 | 75.39 | O |
| ATOM | 8799 | N | HIS | A | 594 | 6.431 | 42.645 | −0.221 | 1.00 | 76.34 | N |
| ATOM | 8801 | CA | HIS | A | 594 | 5.581 | 41.688 | −0.899 | 1.00 | 78.01 | C |
| ATOM | 8803 | CB | HIS | A | 594 | 4.684 | 42.369 | −1.960 | 1.00 | 80.52 | C |
| ATOM | 8806 | CG | HIS | A | 594 | 3.723 | 43.390 | −1.407 | 1.00 | 81.95 | C |
| ATOM | 8807 | ND1 | HIS | A | 594 | 2.483 | 43.052 | −0.907 | 1.00 | 83.85 | N |
| ATOM | 8809 | CE1 | HIS | A | 594 | 1.864 | 44.144 | −0.490 | 1.00 | 84.93 | C |
| ATOM | 8811 | NE2 | HIS | A | 594 | 2.657 | 45.179 | −0.700 | 1.00 | 84.02 | N |
| ATOM | 8813 | CD2 | HIS | A | 594 | 3.827 | 44.736 | −1.271 | 1.00 | 81.36 | C |
| ATOM | 8815 | C | HIS | A | 594 | 6.429 | 40.639 | −1.555 | 1.00 | 77.43 | C |
| ATOM | 8816 | O | HIS | A | 594 | 7.563 | 40.878 | −1.901 | 1.00 | 76.36 | O |
| ATOM | 8817 | N | LEU | A | 595 | 5.821 | 39.480 | −1.748 | 1.00 | 78.64 | N |
| ATOM | 8819 | CA | LEU | A | 595 | 6.443 | 38.324 | −2.362 | 1.00 | 78.53 | C |
| ATOM | 8821 | CB | LEU | A | 595 | 5.448 | 37.149 | −2.299 | 1.00 | 79.33 | C |
| ATOM | 8824 | CG | LEU | A | 595 | 5.801 | 35.797 | −1.684 | 1.00 | 78.71 | C |
| ATOM | 8826 | CD1 | LEU | A | 595 | 6.898 | 35.909 | −0.658 | 1.00 | 76.77 | C |
| ATOM | 8830 | CD2 | LEU | A | 595 | 4.553 | 35.115 | −1.075 | 1.00 | 80.22 | C |
| ATOM | 8834 | C | LEU | A | 595 | 6.829 | 38.606 | −3.822 | 1.00 | 80.06 | C |
| ATOM | 8835 | O | LEU | A | 595 | 6.092 | 39.243 | −4.551 | 1.00 | 81.01 | O |
| ATOM | 8836 | N | CYS | A | 596 | 7.989 | 38.116 | −4.238 | 1.00 | 80.39 | N |
| ATOM | 8838 | CA | CYS | A | 596 | 8.402 | 38.165 | −5.626 | 1.00 | 82.14 | C |
| ATOM | 8840 | CB | CYS | A | 596 | 9.907 | 37.993 | −5.745 | 1.00 | 81.09 | C |
| ATOM | 8843 | SG | CYS | A | 596 | 10.896 | 39.338 | −5.042 | 1.00 | 82.20 | S |
| ATOM | 8844 | C | CYS | A | 596 | 7.750 | 37.016 | −6.381 | 1.00 | 84.23 | C |
| ATOM | 8845 | O | CYS | A | 596 | 7.182 | 36.099 | −5.758 | 1.00 | 84.81 | O |
| ATOM | 8846 | N | HIS | A | 597 | 7.839 | 37.077 | −7.726 | 1.00 | 85.98 | N |
| ATOM | 8848 | CA | HIS | A | 597 | 7.387 | 35.991 | −8.602 | 1.00 | 86.82 | C |
| ATOM | 8850 | CB | HIS | A | 597 | 7.815 | 36.182 | −10.084 | 1.00 | 88.03 | C |
| ATOM | 8853 | CG | HIS | A | 597 | 6.992 | 35.395 | −11.096 | 1.00 | 91.55 | C |
| ATOM | 8854 | ND1 | HIS | A | 597 | 5.695 | 34.962 | −10.863 | 1.00 | 94.55 | N |
| ATOM | 8856 | CE1 | HIS | A | 597 | 5.231 | 34.335 | −11.930 | 1.00 | 94.09 | C |
| ATOM | 8858 | NE2 | HIS | A | 597 | 6.176 | 34.338 | −12.851 | 1.00 | 94.23 | N |
| ATOM | 8860 | CD2 | HIS | A | 597 | 7.278 | 35.008 | −12.366 | 1.00 | 92.74 | C |
| ATOM | 8862 | C | HIS | A | 597 | 8.102 | 34.818 | −8.053 | 1.00 | 85.24 | C |
| ATOM | 8863 | O | HIS | A | 597 | 9.227 | 34.973 | −7.601 | 1.00 | 82.66 | O |
| ATOM | 8864 | N | PRO | A | 598 | 7.470 | 33.649 | −8.079 | 1.00 | 86.55 | N |
| ATOM | 8865 | CA | PRO | A | 598 | 8.206 | 32.453 | −7.664 | 1.00 | 86.49 | C |
| ATOM | 8867 | CB | PRO | A | 598 | 7.109 | 31.362 | −7.590 | 1.00 | 88.23 | C |
| ATOM | 8870 | CG | PRO | A | 598 | 5.750 | 32.113 | −7.680 | 1.00 | 88.45 | C |
| ATOM | 8873 | CD | PRO | A | 598 | 6.076 | 33.330 | −8.490 | 1.00 | 87.86 | C |
| ATOM | 8876 | C | PRO | A | 598 | 9.361 | 32.186 | −8.678 | 1.00 | 86.52 | C |
| ATOM | 8877 | O | PRO | A | 598 | 10.423 | 31.664 | −8.294 | 1.00 | 85.89 | O |
| ATOM | 8878 | N | ASN | A | 599 | 9.205 | 32.641 | −9.925 | 1.00 | 87.20 | N |
| ATOM | 8880 | CA | ASN | A | 599 | 10.270 | 32.471 | −10.943 | 1.00 | 87.60 | C |
| ATOM | 8882 | CB | ASN | A | 599 | 9.669 | 32.588 | −12.365 | 1.00 | 88.64 | C |
| ATOM | 8885 | CG | ASN | A | 599 | 9.382 | 31.218 | −13.006 | 1.00 | 90.07 | C |
| ATOM | 8886 | OD1 | ASN | A | 599 | 9.676 | 31.000 | −14.178 | 1.00 | 92.33 | O |
| ATOM | 8887 | ND2 | ASN | A | 599 | 8.799 | 30.307 | −12.241 | 1.00 | 88.59 | N |
| ATOM | 8890 | C | ASN | A | 599 | 11.524 | 33.388 | −10.788 | 1.00 | 86.52 | C |
| ATOM | 8891 | O | ASN | A | 599 | 12.405 | 33.364 | −11.639 | 1.00 | 87.32 | O |
| ATOM | 8892 | N | CYS | A | 600 | 11.599 | 34.182 | −9.712 | 1.00 | 84.92 | N |
| ATOM | 8894 | CA | CYS | A | 600 | 12.739 | 35.071 | −9.439 | 1.00 | 83.58 | C |
| ATOM | 8896 | CB | CYS | A | 600 | 12.275 | 36.331 | −8.714 | 1.00 | 83.07 | C |
| ATOM | 8899 | SG | CYS | A | 600 | 11.574 | 37.524 | −9.848 | 1.00 | 86.34 | S |
| ATOM | 8900 | C | CYS | A | 600 | 13.757 | 34.368 | −8.590 | 1.00 | 82.06 | C |
| ATOM | 8901 | O | CYS | A | 600 | 13.838 | 34.583 | −7.390 | 1.00 | 80.61 | O |
| ATOM | 8902 | N | THR | A | 601 | 14.543 | 33.532 | −9.240 | 1.00 | 82.20 | N |
| ATOM | 8904 | CA | THR | A | 601 | 15.540 | 32.732 | −8.580 | 1.00 | 81.19 | C |
| ATOM | 8906 | CB | THR | A | 601 | 16.394 | 31.985 | −9.643 | 1.00 | 82.55 | C |
| ATOM | 8908 | OG1 | THR | A | 601 | 15.581 | 31.098 | −10.403 | 1.00 | 81.59 | O |
| ATOM | 8910 | CG2 | THR | A | 601 | 17.362 | 31.047 | −8.992 | 1.00 | 82.82 | C |
| ATOM | 8914 | C | THR | A | 601 | 16.462 | 33.557 | −7.683 | 1.00 | 80.12 | C |
| ATOM | 8915 | O | THR | A | 601 | 17.064 | 32.984 | −6.775 | 1.00 | 80.18 | O |
| ATOM | 8916 | N | TYR | A | 602 | 16.626 | 34.861 | −7.927 | 1.00 | 79.32 | N |
| ATOM | 8918 | CA | TYR | A | 602 | 17.704 | 35.596 | −7.243 | 1.00 | 78.82 | C |
| ATOM | 8920 | CB | TYR | A | 602 | 18.764 | 35.995 | −8.249 | 1.00 | 80.35 | C |
| ATOM | 8923 | CG | TYR | A | 602 | 19.511 | 34.824 | −8.832 | 1.00 | 81.46 | C |
| ATOM | 8924 | CD1 | TYR | A | 602 | 19.213 | 34.362 | −10.117 | 1.00 | 80.69 | C |
| ATOM | 8926 | CE1 | TYR | A | 602 | 19.887 | 33.321 | −10.666 | 1.00 | 81.66 | C |
| ATOM | 8928 | CZ | TYR | A | 602 | 20.870 | 32.693 | −9.952 | 1.00 | 82.09 | C |
| ATOM | 8929 | OH | TYR | A | 602 | 21.510 | 31.618 | −10.515 | 1.00 | 83.96 | O |
| ATOM | 8931 | CE2 | TYR | A | 602 | 21.197 | 33.123 | −8.653 | 1.00 | 82.70 | C |
| ATOM | 8933 | CD2 | TYR | A | 602 | 20.518 | 34.187 | −8.110 | 1.00 | 80.31 | C |
| ATOM | 8935 | C | TYR | A | 602 | 17.314 | 36.830 | −6.455 | 1.00 | 77.63 | C |
| ATOM | 8936 | O | TYR | A | 602 | 18.181 | 37.605 | −6.070 | 1.00 | 76.60 | O |
| ATOM | 8937 | N | GLY | A | 603 | 16.021 | 36.994 | −6.190 | 1.00 | 77.48 | N |
| ATOM | 8939 | CA | GLY | A | 603 | 15.508 | 38.166 | −5.496 | 1.00 | 76.91 | C |
| ATOM | 8942 | C | GLY | A | 603 | 15.009 | 39.185 | −6.473 | 1.00 | 77.91 | C |
| ATOM | 8943 | O | GLY | A | 603 | 15.253 | 39.045 | −7.663 | 1.00 | 78.71 | O |
| ATOM | 8944 | N | CYS | A | 604 | 14.312 | 40.206 | −5.982 | 1.00 | 78.21 | N |
| ATOM | 8946 | CA | CYS | A | 604 | 13.738 | 41.210 | −6.882 | 1.00 | 79.72 | C |
| ATOM | 8948 | CB | CYS | A | 604 | 12.414 | 40.701 | −7.464 | 1.00 | 80.28 | C |
| ATOM | 8951 | SG | CYS | A | 604 | 10.973 | 40.882 | −6.410 | 1.00 | 81.56 | S |
| ATOM | 8952 | C | CYS | A | 604 | 13.558 | 42.674 | −6.440 | 1.00 | 80.19 | C |
| ATOM | 8953 | O | CYS | A | 604 | 13.418 | 43.047 | −5.273 | 1.00 | 77.79 | O |
| ATOM | 8954 | N | THR | A | 605 | 13.512 | 43.461 | −7.504 | 1.00 | 83.00 | N |
| ATOM | 8956 | CA | THR | A | 605 | 13.431 | 44.905 | −7.548 | 1.00 | 84.81 | C |
| ATOM | 8958 | CB | THR | A | 605 | 14.006 | 45.292 | −8.941 | 1.00 | 87.10 | C |
| ATOM | 8960 | OG1 | THR | A | 605 | 15.372 | 45.651 | −8.743 | 1.00 | 89.61 | O |
| ATOM | 8962 | CG2 | THR | A | 605 | 13.337 | 46.510 | −9.660 | 1.00 | 90.11 | C |
| ATOM | 8966 | C | THR | A | 605 | 11.986 | 45.326 | −7.333 | 1.00 | 85.27 | C |
| ATOM | 8967 | O | THR | A | 605 | 11.718 | 46.227 | −6.541 | 1.00 | 85.22 | O |
| ATOM | 8968 | N | GLY | A | 606 | 11.069 | 44.625 | −7.999 | 1.00 | 85.73 | N |
| ATOM | 8970 | CA | GLY | A | 606 | 9.649 | 44.898 | −7.928 | 1.00 | 86.94 | C |
| ATOM | 8973 | C | GLY | A | 606 | 8.783 | 43.657 | −8.124 | 1.00 | 86.94 | C |
| ATOM | 8974 | O | GLY | A | 606 | 9.223 | 42.537 | −7.925 | 1.00 | 85.56 | O |
| ATOM | 8975 | N | PRO | A | 607 | 7.534 | 43.854 | −8.515 | 1.00 | 89.16 | N |
| ATOM | 8976 | CA | PRO | A | 607 | 6.552 | 42.772 | −8.584 | 1.00 | 89.64 | C |
| ATOM | 8978 | CB | PRO | A | 607 | 5.207 | 43.521 | −8.508 | 1.00 | 91.72 | C |
| ATOM | 8981 | CG | PRO | A | 607 | 5.467 | 44.862 | −9.127 | 1.00 | 93.44 | C |
| ATOM | 8984 | CD | PRO | A | 607 | 6.939 | 45.149 | −8.880 | 1.00 | 92.13 | C |
| ATOM | 8987 | C | PRO | A | 607 | 6.562 | 41.858 | −9.835 | 1.00 | 90.94 | C |
| ATOM | 8988 | O | PRO | A | 607 | 6.927 | 42.251 | −10.964 | 1.00 | 91.02 | O |
| ATOM | 8989 | N | GLY | A | 608 | 6.123 | 40.616 | −9.558 | 1.00 | 91.39 | N |
| ATOM | 8991 | CA | GLY | A | 608 | 5.924 | 39.533 | −10.509 | 1.00 | 92.22 | C |
| ATOM | 8994 | C | GLY | A | 608 | 6.989 | 39.311 | −11.589 | 1.00 | 92.72 | C |
| ATOM | 8995 | O | GLY | A | 608 | 8.175 | 39.081 | −11.362 | 1.00 | 91.98 | O |
| ATOM | 8996 | N | LEU | A | 609 | 6.506 | 39.353 | −12.807 | 1.00 | 94.71 | N |
| ATOM | 8998 | CA | LEU | A | 609 | 7.270 | 39.067 | −14.030 | 1.00 | 95.05 | C |
| ATOM | 9000 | CB | LEU | A | 609 | 6.311 | 39.435 | −15.240 | 1.00 | 97.40 | C |
| ATOM | 9003 | CG | LEU | A | 609 | 4.763 | 39.599 | −14.971 | 1.00 | 97.53 | C |
| ATOM | 9005 | CD1 | LEU | A | 609 | 4.034 | 40.291 | −16.098 | 1.00 | 99.87 | C |
| ATOM | 9009 | CD2 | LEU | A | 609 | 4.038 | 38.239 | −14.601 | 1.00 | 96.75 | C |
| ATOM | 9013 | C | LEU | A | 609 | 8.667 | 39.823 | −14.097 | 1.00 | 94.70 | C |
| ATOM | 9014 | O | LEU | A | 609 | 9.722 | 39.184 | −14.227 | 1.00 | 92.65 | O |
| ATOM | 9015 | N | ALA | A | 610 | 8.610 | 41.172 | −13.954 | 1.00 | 96.08 | N |
| ATOM | 9017 | CA | ALA | A | 610 | 9.684 | 42.152 | −14.257 | 1.00 | 96.49 | C |
| ATOM | 9019 | CB | ALA | A | 610 | 9.147 | 43.190 | −15.238 | 1.00 | 98.58 | C |
| ATOM | 9023 | C | ALA | A | 610 | 10.302 | 42.887 | −13.030 | 1.00 | 95.94 | C |
| ATOM | 9024 | O | ALA | A | 610 | 10.840 | 43.987 | −13.157 | 1.00 | 97.11 | O |
| ATOM | 9025 | N | GLY | A | 611 | 10.220 | 42.304 | −11.844 | 1.00 | 94.30 | N |
| ATOM | 9027 | CA | GLY | A | 611 | 11.240 | 42.583 | −10.848 | 1.00 | 93.78 | C |
| ATOM | 9030 | C | GLY | A | 611 | 12.516 | 41.826 | −11.256 | 1.00 | 94.52 | C |
| ATOM | 9031 | O | GLY | A | 611 | 13.637 | 42.114 | −10.819 | 1.00 | 93.76 | O |
| ATOM | 9032 | N | CYS | A | 612 | 12.314 | 40.804 | −12.087 | 1.00 | 96.55 | N |
| ATOM | 9034 | CA | CYS | A | 612 | 13.377 | 40.054 | −12.743 | 1.00 | 97.90 | C |
| ATOM | 9036 | CB | CYS | A | 612 | 12.937 | 38.613 | −12.854 | 1.00 | 96.30 | C |
| ATOM | 9039 | SG | CYS | A | 612 | 13.031 | 37.903 | −11.240 | 1.00 | 90.82 | S |
| ATOM | 9040 | C | CYS | A | 612 | 13.582 | 40.653 | −14.140 | 1.00 | 103.59 | C |
| ATOM | 9041 | O | CYS | A | 612 | 12.645 | 40.648 | −14.971 | 1.00 | 103.99 | O |
| ATOM | 9042 | N | PRO | A | 613 | 14.763 | 41.243 | −14.370 | 1.00 | 108.80 | N |
| ATOM | 9043 | CA | PRO | A | 613 | 15.182 | 41.615 | −15.726 | 1.00 | 114.87 | C |
| ATOM | 9045 | CB | PRO | A | 613 | 15.737 | 43.030 | −15.521 | 1.00 | 114.53 | C |
| ATOM | 9048 | CG | PRO | A | 613 | 16.295 | 42.984 | −14.088 | 1.00 | 110.83 | C |
| ATOM | 9051 | CD | PRO | A | 613 | 15.772 | 41.685 | −13.378 | 1.00 | 107.45 | C |
| ATOM | 9054 | C | PRO | A | 613 | 16.262 | 40.618 | −16.273 | 1.00 | 122.10 | C |
| ATOM | 9055 | O | PRO | A | 613 | 16.937 | 39.895 | −15.470 | 1.00 | 120.24 | O |
| ATOM | 9056 | N | THR | A | 614 | 16.391 | 40.617 | −17.621 | 1.00 | 47.89 | N |
| ATOM | 9058 | CA | THR | A | 614 | 17.198 | 39.690 | −18.467 | 1.00 | 53.92 | C |
| ATOM | 9060 | CB | THR | A | 614 | 18.247 | 38.823 | −17.659 | 1.00 | 55.51 | C |
| ATOM | 9062 | OG1 | THR | A | 614 | 19.340 | 39.668 | −17.217 | 1.00 | 58.77 | O |
| ATOM | 9064 | CG2 | THR | A | 614 | 18.907 | 37.587 | −18.539 | 1.00 | 57.00 | C |
| ATOM | 9068 | C | THR | A | 614 | 16.172 | 38.798 | −19.222 | 1.00 | 56.93 | C |
| ATOM | 9069 | O | THR | A | 614 | 15.663 | 39.166 | −20.337 | 1.00 | 59.17 | O |
| ATOM | 9070 | OXT | THR | A | 614 | 15.847 | 37.720 | −18.669 | 1.00 | 59.96 | O |
| ATOM | 9071 | N | ASP | C | 1 | 33.389 | 65.342 | 66.616 | 1.00 | 67.77 | N |
| ATOM | 9073 | CA | ASP | C | 1 | 33.591 | 64.060 | 67.391 | 1.00 | 63.04 | C |
| ATOM | 9075 | CB | ASP | C | 1 | 34.960 | 64.034 | 68.124 | 1.00 | 62.38 | C |
| ATOM | 9078 | CG | ASP | C | 1 | 36.160 | 63.729 | 67.215 | 1.00 | 63.30 | C |
| ATOM | 9079 | OD1 | ASP | C | 1 | 36.329 | 62.559 | 66.749 | 1.00 | 62.63 | O |
| ATOM | 9080 | OD2 | ASP | C | 1 | 37.047 | 64.606 | 67.012 | 1.00 | 67.29 | O |
| ATOM | 9081 | C | ASP | C | 1 | 32.479 | 63.876 | 68.450 | 1.00 | 60.60 | C |
| ATOM | 9082 | O | ASP | C | 1 | 31.963 | 64.812 | 68.966 | 1.00 | 63.19 | O |
| ATOM | 9085 | N | ILE | C | 2 | 32.190 | 62.647 | 68.811 | 1.00 | 56.23 | N |
| ATOM | 9087 | CA | ILE | C | 2 | 31.119 | 62.338 | 69.720 | 1.00 | 54.21 | C |
| ATOM | 9089 | CB | ILE | C | 2 | 30.653 | 60.829 | 69.431 | 1.00 | 51.58 | C |
| ATOM | 9091 | CG1 | ILE | C | 2 | 29.523 | 60.345 | 70.377 | 1.00 | 50.81 | C |
| ATOM | 9094 | CD1 | ILE | C | 2 | 28.466 | 59.526 | 69.644 | 1.00 | 49.41 | C |
| ATOM | 9098 | CG2 | ILE | C | 2 | 31.758 | 59.838 | 69.497 | 1.00 | 46.09 | C |
| ATOM | 9102 | C | ILE | C | 2 | 31.538 | 62.546 | 71.167 | 1.00 | 52.71 | C |
| ATOM | 9103 | O | ILE | C | 2 | 32.470 | 61.928 | 71.639 | 1.00 | 50.84 | O |
| ATOM | 9104 | N | LEU | C | 3 | 30.821 | 63.383 | 71.886 | 1.00 | 54.32 | N |
| ATOM | 9106 | CA | LEU | C | 3 | 31.006 | 63.509 | 73.329 | 1.00 | 53.38 | C |
| ATOM | 9108 | CB | LEU | C | 3 | 30.547 | 64.886 | 73.785 | 1.00 | 57.50 | C |
| ATOM | 9111 | CG | LEU | C | 3 | 31.316 | 66.005 | 73.123 | 1.00 | 61.79 | C |
| ATOM | 9113 | CD1 | LEU | C | 3 | 31.128 | 67.318 | 73.861 | 1.00 | 66.05 | C |
| ATOM | 9117 | CD2 | LEU | C | 3 | 32.806 | 65.586 | 73.107 | 1.00 | 61.67 | C |
| ATOM | 9121 | C | LEU | C | 3 | 30.206 | 62.515 | 74.091 | 1.00 | 50.57 | C |
| ATOM | 9122 | O | LEU | C | 3 | 29.205 | 62.077 | 73.632 | 1.00 | 50.19 | O |
| ATOM | 9123 | N | LEU | C | 4 | 30.655 | 62.200 | 75.294 | 1.00 | 49.48 | N |
| ATOM | 9125 | CA | LEU | C | 4 | 29.977 | 61.272 | 76.210 | 1.00 | 47.62 | C |
| ATOM | 9127 | CB | LEU | C | 4 | 30.661 | 59.899 | 76.270 | 1.00 | 44.26 | C |
| ATOM | 9130 | CG | LEU | C | 4 | 30.556 | 58.903 | 75.095 | 1.00 | 42.54 | C |
| ATOM | 9132 | CD1 | LEU | C | 4 | 31.192 | 57.536 | 75.461 | 1.00 | 42.27 | C |
| ATOM | 9136 | CD2 | LEU | C | 4 | 29.161 | 58.642 | 74.668 | 1.00 | 41.09 | C |
| ATOM | 9140 | C | LEU | C | 4 | 30.102 | 61.939 | 77.564 | 1.00 | 49.23 | C |
| ATOM | 9141 | O | LEU | C | 4 | 31.203 | 62.184 | 78.046 | 1.00 | 50.89 | O |
| ATOM | 9142 | N | THR | C | 5 | 28.982 | 62.206 | 78.193 | 1.00 | 50.08 | N |
| ATOM | 9144 | CA | THR | C | 5 | 28.925 | 63.052 | 79.340 | 1.00 | 51.09 | C |
| ATOM | 9146 | CB | THR | C | 5 | 28.023 | 64.197 | 78.914 | 1.00 | 54.47 | C |
| ATOM | 9148 | OG1 | THR | C | 5 | 28.669 | 64.889 | 77.847 | 1.00 | 54.99 | O |
| ATOM | 9150 | CG2 | THR | C | 5 | 27.841 | 65.257 | 80.010 | 1.00 | 58.10 | C |
| ATOM | 9154 | C | THR | C | 5 | 28.318 | 62.222 | 80.455 | 1.00 | 50.26 | C |
| ATOM | 9155 | O | THR | C | 5 | 27.173 | 61.791 | 80.329 | 1.00 | 52.74 | O |
| ATOM | 9156 | N | GLN | C | 6 | 29.054 | 61.916 | 81.509 | 1.00 | 48.92 | N |
| ATOM | 9158 | CA | GLN | C | 6 | 28.477 | 61.131 | 82.612 | 1.00 | 48.41 | C |
| ATOM | 9160 | CB | GLN | C | 6 | 29.420 | 60.029 | 83.106 | 1.00 | 46.13 | C |
| ATOM | 9163 | CG | GLN | C | 6 | 29.841 | 58.951 | 82.032 | 1.00 | 46.01 | C |
| ATOM | 9166 | CD | GLN | C | 6 | 30.748 | 57.811 | 82.605 | 1.00 | 42.83 | C |
| ATOM | 9167 | OE1 | GLN | C | 6 | 30.341 | 57.108 | 83.506 | 1.00 | 43.11 | O |
| ATOM | 9168 | NE2 | GLN | C | 6 | 31.931 | 57.649 | 82.063 | 1.00 | 39.15 | N |
| ATOM | 9171 | C | GLN | C | 6 | 28.086 | 62.057 | 83.776 | 1.00 | 51.15 | C |
| ATOM | 9172 | O | GLN | C | 6 | 28.575 | 63.185 | 83.915 | 1.00 | 51.19 | O |
| ATOM | 9173 | N | SER | C | 7 | 27.195 | 61.530 | 84.618 | 1.00 | 52.05 | N |
| ATOM | 9175 | CA | SER | C | 7 | 26.399 | 62.344 | 85.524 | 1.00 | 55.11 | C |
| ATOM | 9177 | CB | SER | C | 7 | 25.240 | 62.929 | 84.689 | 1.00 | 57.59 | C |
| ATOM | 9180 | OG | SER | C | 7 | 25.571 | 64.267 | 84.306 | 1.00 | 63.19 | O |
| ATOM | 9182 | C | SER | C | 7 | 25.843 | 61.474 | 86.652 | 1.00 | 54.25 | C |
| ATOM | 9183 | O | SER | C | 7 | 25.344 | 60.419 | 86.357 | 1.00 | 53.75 | O |
| ATOM | 9184 | N | PRO | C | 8 | 25.896 | 61.871 | 87.925 | 1.00 | 55.03 | N |
| ATOM | 9185 | CA | PRO | C | 8 | 26.657 | 63.025 | 88.411 | 1.00 | 56.11 | C |
| ATOM | 9187 | CB | PRO | C | 8 | 26.204 | 63.135 | 89.865 | 1.00 | 57.98 | C |
| ATOM | 9190 | CG | PRO | C | 8 | 25.667 | 61.770 | 90.233 | 1.00 | 56.40 | C |
| ATOM | 9193 | CD | PRO | C | 8 | 25.101 | 61.220 | 88.994 | 1.00 | 55.40 | C |
| ATOM | 9196 | C | PRO | C | 8 | 28.134 | 62.756 | 88.412 | 1.00 | 54.47 | C |
| ATOM | 9197 | O | PRO | C | 8 | 28.565 | 61.631 | 88.075 | 1.00 | 52.22 | O |
| ATOM | 9198 | N | VAL | C | 9 | 28.908 | 63.726 | 88.859 | 1.00 | 55.53 | N |
| ATOM | 9200 | CA | VAL | C | 9 | 30.311 | 63.456 | 89.017 | 1.00 | 55.24 | C |
| ATOM | 9202 | CB | VAL | C | 9 | 31.120 | 64.738 | 89.253 | 1.00 | 58.63 | C |
| ATOM | 9204 | CG1 | VAL | C | 9 | 32.588 | 64.416 | 89.781 | 1.00 | 58.03 | C |
| ATOM | 9208 | CG2 | VAL | C | 9 | 31.166 | 65.580 | 87.925 | 1.00 | 60.37 | C |
| ATOM | 9212 | C | VAL | C | 9 | 30.574 | 62.469 | 90.150 | 1.00 | 54.36 | C |
| ATOM | 9213 | O | VAL | C | 9 | 31.400 | 61.558 | 89.968 | 1.00 | 53.35 | O |
| ATOM | 9214 | N | ILE | C | 10 | 29.927 | 62.686 | 91.302 | 1.00 | 55.71 | N |
| ATOM | 9216 | CA | ILE | C | 10 | 29.879 | 61.722 | 92.418 | 1.00 | 55.79 | C |
| ATOM | 9218 | CB | ILE | C | 10 | 30.485 | 62.359 | 93.690 | 1.00 | 57.92 | C |
| ATOM | 9220 | CG1 | ILE | C | 10 | 32.000 | 62.430 | 93.602 | 1.00 | 59.27 | C |
| ATOM | 9223 | CD1 | ILE | C | 10 | 32.638 | 63.164 | 94.791 | 1.00 | 62.22 | C |
| ATOM | 9227 | CG2 | ILE | C | 10 | 30.200 | 61.541 | 94.922 | 1.00 | 58.67 | C |
| ATOM | 9231 | C | ILE | C | 10 | 28.393 | 61.258 | 92.677 | 1.00 | 57.07 | C |
| ATOM | 9232 | O | ILE | C | 10 | 27.464 | 62.062 | 92.664 | 1.00 | 60.01 | O |
| ATOM | 9233 | N | LEU | C | 11 | 28.137 | 59.983 | 92.929 | 1.00 | 55.55 | N |
| ATOM | 9235 | CA | LEU | C | 11 | 26.769 | 59.588 | 93.190 | 1.00 | 56.88 | C |
| ATOM | 9237 | CB | LEU | C | 11 | 26.419 | 58.508 | 92.202 | 1.00 | 54.80 | C |
| ATOM | 9240 | CG | LEU | C | 11 | 25.025 | 57.907 | 92.195 | 1.00 | 55.67 | C |
| ATOM | 9242 | CD1 | LEU | C | 11 | 24.004 | 58.957 | 92.115 | 1.00 | 58.93 | C |
| ATOM | 9246 | CD2 | LEU | C | 11 | 24.914 | 56.991 | 90.986 | 1.00 | 54.73 | C |
| ATOM | 9250 | C | LEU | C | 11 | 26.688 | 59.068 | 94.620 | 1.00 | 59.06 | C |
| ATOM | 9251 | O | LEU | C | 11 | 27.287 | 58.041 | 94.901 | 1.00 | 59.11 | O |
| ATOM | 9252 | N | SER | C | 12 | 26.023 | 59.782 | 95.533 | 1.00 | 61.71 | N |
| ATOM | 9254 | CA | SER | C | 12 | 26.005 | 59.382 | 96.956 | 1.00 | 63.27 | C |
| ATOM | 9256 | CB | SER | C | 12 | 26.214 | 60.556 | 97.941 | 1.00 | 65.33 | C |
| ATOM | 9259 | OG | SER | C | 12 | 27.431 | 61.153 | 97.699 | 1.00 | 63.03 | O |
| ATOM | 9261 | C | SER | C | 12 | 24.681 | 58.744 | 97.285 | 1.00 | 64.64 | C |
| ATOM | 9262 | O | SER | C | 12 | 23.656 | 59.375 | 97.191 | 1.00 | 66.78 | O |
| ATOM | 9263 | N | VAL | C | 13 | 24.750 | 57.540 | 97.814 | 1.00 | 64.40 | N |
| ATOM | 9265 | CA | VAL | C | 13 | 23.657 | 56.618 | 97.810 | 1.00 | 65.81 | C |
| ATOM | 9267 | CB | VAL | C | 13 | 23.891 | 55.757 | 96.586 | 1.00 | 63.63 | C |
| ATOM | 9269 | CG1 | VAL | C | 13 | 23.223 | 54.454 | 96.715 | 1.00 | 66.53 | C |
| ATOM | 9273 | CG2 | VAL | C | 13 | 23.399 | 56.484 | 95.355 | 1.00 | 64.60 | C |
| ATOM | 9277 | C | VAL | C | 13 | 23.712 | 55.741 | 99.037 | 1.00 | 66.60 | C |
| ATOM | 9278 | O | VAL | C | 13 | 24.785 | 55.268 | 99.380 | 1.00 | 64.51 | O |
| ATOM | 9279 | N | SER | C | 14 | 22.579 | 55.475 | 99.680 | 1.00 | 69.64 | N |
| ATOM | 9281 | CA | SER | C | 14 | 22.601 | 54.560 | 100.836 | 1.00 | 72.09 | C |
| ATOM | 9283 | CB | SER | C | 14 | 21.478 | 54.804 | 101.839 | 1.00 | 76.76 | C |
| ATOM | 9286 | OG | SER | C | 14 | 20.810 | 56.030 | 101.609 | 1.00 | 79.16 | O |
| ATOM | 9288 | C | SER | C | 14 | 22.599 | 53.119 | 100.356 | 1.00 | 70.71 | C |
| ATOM | 9289 | O | SER | C | 14 | 22.149 | 52.842 | 99.275 | 1.00 | 68.37 | O |
| ATOM | 9290 | N | PRO | C | 15 | 23.152 | 52.211 | 101.149 | 1.00 | 72.08 | N |
| ATOM | 9291 | CA | PRO | C | 15 | 23.353 | 50.835 | 100.735 | 1.00 | 71.76 | C |
| ATOM | 9293 | CB | PRO | C | 15 | 24.297 | 50.294 | 101.808 | 1.00 | 73.08 | C |
| ATOM | 9296 | CG | PRO | C | 15 | 23.978 | 51.010 | 102.986 | 1.00 | 76.10 | C |
| ATOM | 9299 | CD | PRO | C | 15 | 23.611 | 52.399 | 102.530 | 1.00 | 74.92 | C |
| ATOM | 9302 | C | PRO | C | 15 | 22.056 | 50.082 | 100.765 | 1.00 | 75.00 | C |
| ATOM | 9303 | O | PRO | C | 15 | 21.340 | 50.172 | 101.721 | 1.00 | 79.23 | O |
| ATOM | 9304 | N | GLY | C | 16 | 21.755 | 49.355 | 99.712 | 1.00 | 74.43 | N |
| ATOM | 9306 | CA | GLY | C | 16 | 20.459 | 48.743 | 99.583 | 1.00 | 77.95 | C |
| ATOM | 9309 | C | GLY | C | 16 | 19.586 | 49.408 | 98.540 | 1.00 | 77.45 | C |
| ATOM | 9310 | O | GLY | C | 16 | 18.688 | 48.741 | 98.034 | 1.00 | 80.57 | O |
| ATOM | 9311 | N | GLU | C | 17 | 19.809 | 50.673 | 98.194 | 1.00 | 75.10 | N |
| ATOM | 9313 | CA | GLU | C | 17 | 18.992 | 51.329 | 97.158 | 1.00 | 74.75 | C |
| ATOM | 9315 | CB | GLU | C | 17 | 19.245 | 52.794 | 97.143 | 1.00 | 73.81 | C |
| ATOM | 9318 | CG | GLU | C | 17 | 18.823 | 53.497 | 98.389 | 1.00 | 79.22 | C |
| ATOM | 9321 | CD | GLU | C | 17 | 18.808 | 54.995 | 98.145 | 1.00 | 79.79 | C |
| ATOM | 9322 | OE1 | GLU | C | 17 | 19.851 | 55.616 | 97.710 | 1.00 | 69.50 | O |
| ATOM | 9323 | OE2 | GLU | C | 17 | 17.681 | 55.505 | 98.369 | 1.00 | 88.18 | O |
| ATOM | 9324 | C | GLU | C | 17 | 19.231 | 50.930 | 95.713 | 1.00 | 71.55 | C |
| ATOM | 9325 | O | GLU | C | 17 | 20.338 | 50.462 | 95.351 | 1.00 | 66.99 | O |
| ATOM | 9326 | N | ALA | C | 18 | 18.177 | 51.167 | 94.895 | 1.00 | 72.93 | N |
| ATOM | 9328 | CA | ALA | C | 18 | 18.342 | 51.297 | 93.452 | 1.00 | 69.72 | C |
| ATOM | 9330 | CB | ALA | C | 18 | 17.040 | 51.182 | 92.640 | 1.00 | 70.80 | C |
| ATOM | 9334 | C | ALA | C | 18 | 19.087 | 52.642 | 93.212 | 1.00 | 67.33 | C |
| ATOM | 9335 | O | ALA | C | 18 | 18.731 | 53.667 | 93.755 | 1.00 | 69.28 | O |
| ATOM | 9336 | N | VAL | C | 19 | 20.151 | 52.542 | 92.424 | 1.00 | 63.49 | N |
| ATOM | 9338 | CA | VAL | C | 19 | 21.005 | 53.594 | 91.948 | 1.00 | 61.76 | C |
| ATOM | 9340 | CB | VAL | C | 19 | 22.441 | 53.202 | 92.367 | 1.00 | 59.76 | C |
| ATOM | 9342 | CG1 | VAL | C | 19 | 23.474 | 54.131 | 91.797 | 1.00 | 57.51 | C |
| ATOM | 9346 | CG2 | VAL | C | 19 | 22.521 | 53.203 | 93.815 | 1.00 | 62.73 | C |
| ATOM | 9350 | C | VAL | C | 19 | 21.059 | 53.596 | 90.416 | 1.00 | 59.34 | C |
| ATOM | 9351 | O | VAL | C | 19 | 21.188 | 52.538 | 89.832 | 1.00 | 59.40 | O |
| ATOM | 9352 | N | SER | C | 20 | 21.053 | 54.764 | 89.776 | 1.00 | 57.96 | N |
| ATOM | 9354 | CA | SER | C | 20 | 21.205 | 54.860 | 88.333 | 1.00 | 55.03 | C |
| ATOM | 9356 | CB | SER | C | 20 | 20.027 | 55.545 | 87.683 | 1.00 | 57.01 | C |
| ATOM | 9359 | OG | SER | C | 20 | 18.851 | 54.897 | 88.037 | 1.00 | 61.60 | O |
| ATOM | 9361 | C | SER | C | 20 | 22.298 | 55.767 | 88.035 | 1.00 | 53.24 | C |
| ATOM | 9362 | O | SER | C | 20 | 22.254 | 56.879 | 88.497 | 1.00 | 54.65 | O |
| ATOM | 9363 | N | PHE | C | 21 | 23.257 | 55.326 | 87.212 | 1.00 | 50.84 | N |
| ATOM | 9365 | CA | PHE | C | 21 | 24.247 | 56.198 | 86.595 | 1.00 | 48.38 | C |
| ATOM | 9367 | CB | PHE | C | 21 | 25.592 | 55.519 | 86.453 | 1.00 | 46.09 | C |
| ATOM | 9370 | CG | PHE | C | 21 | 26.047 | 54.812 | 87.647 | 1.00 | 47.07 | C |
| ATOM | 9371 | CD1 | PHE | C | 21 | 25.631 | 53.517 | 87.880 | 1.00 | 50.09 | C |
| ATOM | 9373 | CE1 | PHE | C | 21 | 26.041 | 52.843 | 88.956 | 1.00 | 50.03 | C |
| ATOM | 9375 | CZ | PHE | C | 21 | 26.878 | 53.444 | 89.804 | 1.00 | 52.96 | C |
| ATOM | 9377 | CE2 | PHE | C | 21 | 27.326 | 54.751 | 89.576 | 1.00 | 52.36 | C |
| ATOM | 9379 | CD2 | PHE | C | 21 | 26.921 | 55.399 | 88.494 | 1.00 | 49.70 | C |
| ATOM | 9381 | C | PHE | C | 21 | 23.797 | 56.492 | 85.203 | 1.00 | 47.89 | C |
| ATOM | 9382 | O | PHE | C | 21 | 23.140 | 55.681 | 84.629 | 1.00 | 47.93 | O |
| ATOM | 9383 | N | SER | C | 22 | 24.198 | 57.626 | 84.646 | 1.00 | 48.25 | N |
| ATOM | 9385 | CA | SER | C | 22 | 23.802 | 58.025 | 83.317 | 1.00 | 49.61 | C |
| ATOM | 9387 | CB | SER | C | 22 | 22.694 | 59.023 | 83.416 | 1.00 | 53.37 | C |
| ATOM | 9390 | OG | SER | C | 22 | 23.123 | 59.992 | 84.294 | 1.00 | 55.34 | O |
| ATOM | 9392 | C | SER | C | 22 | 24.848 | 58.684 | 82.473 | 1.00 | 49.38 | C |
| ATOM | 9393 | O | SER | C | 22 | 25.692 | 59.451 | 82.980 | 1.00 | 51.10 | O |
| ATOM | 9394 | N | CYS | C | 23 | 24.693 | 58.465 | 81.165 | 1.00 | 49.64 | N |
| ATOM | 9396 | CA | CYS | C | 23 | 25.639 | 58.792 | 80.105 | 1.00 | 49.37 | C |
| ATOM | 9398 | CB | CYS | C | 23 | 26.354 | 57.470 | 79.749 | 1.00 | 47.09 | C |
| ATOM | 9401 | SG | CYS | C | 23 | 27.565 | 57.489 | 78.438 | 1.00 | 48.27 | S |
| ATOM | 9402 | C | CYS | C | 23 | 24.845 | 59.312 | 78.891 | 1.00 | 51.54 | C |
| ATOM | 9403 | O | CYS | C | 23 | 23.923 | 58.637 | 78.433 | 1.00 | 51.51 | O |
| ATOM | 9404 | N | ARG | C | 24 | 25.162 | 60.511 | 78.383 | 1.00 | 53.04 | N |
| ATOM | 9406 | CA | ARG | C | 24 | 24.403 | 61.116 | 77.240 | 1.00 | 54.66 | C |
| ATOM | 9408 | CB | ARG | C | 24 | 23.681 | 62.434 | 77.585 | 1.00 | 56.60 | C |
| ATOM | 9417 | C | ARG | C | 24 | 25.410 | 61.393 | 76.163 | 1.00 | 53.77 | C |
| ATOM | 9418 | O | ARG | C | 24 | 26.331 | 62.193 | 76.410 | 1.00 | 55.68 | O |
| ATOM | 9419 | N | ALA | C | 25 | 25.276 | 60.747 | 75.000 | 1.00 | 51.76 | N |
| ATOM | 9421 | CA | ALA | C | 25 | 26.118 | 61.042 | 73.835 | 1.00 | 50.58 | C |
| ATOM | 9423 | CB | ALA | C | 25 | 26.241 | 59.816 | 72.982 | 1.00 | 48.78 | C |
| ATOM | 9427 | C | ALA | C | 25 | 25.591 | 62.193 | 73.012 | 1.00 | 53.60 | C |
| ATOM | 9428 | O | ALA | C | 25 | 24.412 | 62.317 | 72.863 | 1.00 | 56.32 | O |
| ATOM | 9429 | N | SER | C | 26 | 26.483 | 63.018 | 72.497 | 1.00 | 54.27 | N |
| ATOM | 9431 | CA | SER | C | 26 | 26.146 | 64.197 | 71.725 | 1.00 | 59.13 | C |
| ATOM | 9433 | CB | SER | C | 26 | 27.425 | 64.981 | 71.463 | 1.00 | 59.86 | C |
| ATOM | 9436 | OG | SER | C | 26 | 28.278 | 64.302 | 70.552 | 1.00 | 59.91 | O |
| ATOM | 9438 | C | SER | C | 26 | 25.446 | 63.922 | 70.377 | 1.00 | 61.50 | C |
| ATOM | 9439 | O | SER | C | 26 | 24.883 | 64.864 | 69.768 | 1.00 | 66.20 | O |
| ATOM | 9440 | N | GLN | C | 27 | 25.529 | 62.670 | 69.901 | 1.00 | 58.24 | N |
| ATOM | 9442 | CA | GLN | C | 27 | 24.827 | 62.225 | 68.706 | 1.00 | 60.10 | C |
| ATOM | 9444 | CB | GLN | C | 27 | 25.688 | 62.296 | 67.470 | 1.00 | 59.83 | C |
| ATOM | 9447 | CG | GLN | C | 27 | 27.081 | 62.008 | 67.716 | 1.00 | 58.23 | C |
| ATOM | 9450 | CD | GLN | C | 27 | 27.918 | 61.992 | 66.456 | 1.00 | 60.02 | C |
| ATOM | 9451 | OE1 | GLN | C | 27 | 28.552 | 62.988 | 66.171 | 1.00 | 62.47 | O |
| ATOM | 9452 | NE2 | GLN | C | 27 | 27.924 | 60.869 | 65.701 | 1.00 | 58.88 | N |
| ATOM | 9455 | C | GLN | C | 27 | 24.312 | 60.827 | 68.888 | 1.00 | 57.93 | C |
| ATOM | 9456 | O | GLN | C | 27 | 24.902 | 60.058 | 69.598 | 1.00 | 53.57 | O |
| ATOM | 9457 | N | SER | C | 28 | 23.165 | 60.527 | 68.271 | 1.00 | 61.55 | N |
| ATOM | 9459 | CA | SER | C | 28 | 22.408 | 59.316 | 68.582 | 1.00 | 60.90 | C |
| ATOM | 9461 | CB | SER | C | 28 | 20.945 | 59.423 | 68.203 | 1.00 | 65.18 | C |
| ATOM | 9464 | OG | SER | C | 28 | 20.591 | 58.143 | 67.697 | 1.00 | 66.79 | O |
| ATOM | 9466 | C | SER | C | 28 | 23.004 | 58.084 | 67.889 | 1.00 | 58.36 | C |
| ATOM | 9467 | O | SER | C | 28 | 23.256 | 58.074 | 66.663 | 1.00 | 60.09 | O |
| ATOM | 9468 | N | ILE | C | 29 | 23.131 | 57.029 | 68.691 | 1.00 | 54.91 | N |
| ATOM | 9470 | CA | ILE | C | 29 | 24.081 | 55.941 | 68.499 | 1.00 | 50.73 | C |
| ATOM | 9472 | CB | ILE | C | 29 | 24.939 | 56.025 | 69.690 | 1.00 | 48.76 | C |
| ATOM | 9474 | CG1 | ILE | C | 29 | 26.204 | 56.799 | 69.506 | 1.00 | 47.90 | C |
| ATOM | 9477 | CD1 | ILE | C | 29 | 26.781 | 56.887 | 71.020 | 1.00 | 46.20 | C |
| ATOM | 9481 | CG2 | ILE | C | 29 | 25.406 | 54.716 | 70.140 | 1.00 | 50.42 | C |
| ATOM | 9485 | C | ILE | C | 29 | 23.419 | 54.538 | 68.588 | 1.00 | 48.53 | C |
| ATOM | 9486 | O | ILE | C | 29 | 24.082 | 53.548 | 68.607 | 1.00 | 45.78 | O |
| ATOM | 9487 | N | GLY | C | 30 | 22.128 | 54.442 | 68.772 | 1.00 | 50.20 | N |
| ATOM | 9489 | CA | GLY | C | 30 | 21.526 | 53.158 | 68.932 | 1.00 | 49.68 | C |
| ATOM | 9492 | C | GLY | C | 30 | 21.768 | 52.584 | 70.304 | 1.00 | 48.73 | C |
| ATOM | 9493 | O | GLY | C | 30 | 21.368 | 53.115 | 71.357 | 1.00 | 48.59 | O |
| ATOM | 9494 | N | THR | C | 31 | 22.462 | 51.467 | 70.275 | 1.00 | 47.96 | N |
| ATOM | 9496 | CA | THR | C | 31 | 22.650 | 50.599 | 71.412 | 1.00 | 47.77 | C |
| ATOM | 9498 | CB | THR | C | 31 | 22.033 | 49.252 | 71.008 | 1.00 | 50.14 | C |
| ATOM | 9500 | OG1 | THR | C | 31 | 20.656 | 49.459 | 70.720 | 1.00 | 56.14 | O |
| ATOM | 9502 | CG2 | THR | C | 31 | 21.894 | 48.337 | 72.170 | 1.00 | 52.95 | C |
| ATOM | 9506 | C | THR | C | 31 | 24.137 | 50.455 | 71.595 | 1.00 | 44.48 | C |
| ATOM | 9507 | O | THR | C | 31 | 24.637 | 49.717 | 72.445 | 1.00 | 42.22 | O |
| ATOM | 9508 | N | ASN | C | 32 | 24.885 | 51.149 | 70.758 | 1.00 | 43.47 | N |
| ATOM | 9510 | CA | ASN | C | 32 | 26.288 | 50.870 | 70.721 | 1.00 | 41.11 | C |
| ATOM | 9512 | CB | ASN | C | 32 | 26.825 | 51.306 | 69.381 | 1.00 | 41.94 | C |
| ATOM | 9515 | CG | ASN | C | 32 | 26.386 | 50.392 | 68.282 | 1.00 | 43.35 | C |
| ATOM | 9516 | OD1 | ASN | C | 32 | 26.176 | 49.129 | 68.474 | 1.00 | 38.32 | O |
| ATOM | 9517 | ND2 | ASN | C | 32 | 26.245 | 51.004 | 67.090 | 1.00 | 43.19 | N |
| ATOM | 9520 | C | ASN | C | 32 | 27.059 | 51.545 | 71.863 | 1.00 | 39.52 | C |
| ATOM | 9521 | O | ASN | C | 32 | 27.910 | 52.433 | 71.610 | 1.00 | 37.18 | O |
| ATOM | 9522 | N | ILE | C | 33 | 26.740 | 51.112 | 73.093 | 1.00 | 39.04 | N |
| ATOM | 9524 | CA | ILE | C | 33 | 27.444 | 51.546 | 74.289 | 1.00 | 38.42 | C |
| ATOM | 9526 | CB | ILE | C | 33 | 26.556 | 52.547 | 75.003 | 1.00 | 40.72 | C |
| ATOM | 9528 | CG1 | ILE | C | 33 | 27.221 | 53.922 | 74.939 | 1.00 | 43.37 | C |
| ATOM | 9531 | CD1 | ILE | C | 33 | 26.172 | 54.854 | 74.384 | 1.00 | 49.43 | C |
| ATOM | 9535 | CG2 | ILE | C | 33 | 26.295 | 52.247 | 76.459 | 1.00 | 41.18 | C |
| ATOM | 9539 | C | ILE | C | 33 | 27.769 | 50.425 | 75.211 | 1.00 | 36.61 | C |
| ATOM | 9540 | O | ILE | C | 33 | 26.935 | 49.529 | 75.421 | 1.00 | 38.59 | O |
| ATOM | 9541 | N | HIS | C | 34 | 28.925 | 50.499 | 75.846 | 1.00 | 34.61 | N |
| ATOM | 9543 | CA | HIS | C | 34 | 29.162 | 49.613 | 76.966 | 1.00 | 34.71 | C |
| ATOM | 9545 | CB | HIS | C | 34 | 30.200 | 48.643 | 76.574 | 1.00 | 33.97 | C |
| ATOM | 9548 | CG | HIS | C | 34 | 30.194 | 48.311 | 75.141 | 1.00 | 34.26 | C |
| ATOM | 9549 | ND1 | HIS | C | 34 | 29.367 | 47.348 | 74.630 | 1.00 | 38.92 | N |
| ATOM | 9551 | CE1 | HIS | C | 34 | 29.607 | 47.205 | 73.339 | 1.00 | 38.14 | C |
| ATOM | 9553 | NE2 | HIS | C | 34 | 30.540 | 48.071 | 72.995 | 1.00 | 37.85 | N |
| ATOM | 9555 | CD2 | HIS | C | 34 | 30.930 | 48.773 | 74.108 | 1.00 | 34.95 | C |
| ATOM | 9557 | C | HIS | C | 34 | 29.522 | 50.260 | 78.342 | 1.00 | 34.32 | C |
| ATOM | 9558 | O | HIS | C | 34 | 29.807 | 51.432 | 78.392 | 1.00 | 33.64 | O |
| ATOM | 9559 | N | TRP | C | 35 | 29.445 | 49.465 | 79.426 | 1.00 | 34.27 | N |
| ATOM | 9561 | CA | TRP | C | 35 | 29.783 | 49.896 | 80.759 | 1.00 | 34.35 | C |
| ATOM | 9563 | CB | TRP | C | 35 | 28.560 | 49.879 | 81.571 | 1.00 | 35.41 | C |
| ATOM | 9566 | CG | TRP | C | 35 | 27.514 | 50.870 | 81.176 | 1.00 | 38.77 | C |
| ATOM | 9567 | CD1 | TRP | C | 35 | 26.443 | 50.635 | 80.362 | 1.00 | 38.77 | C |
| ATOM | 9569 | NE1 | TRP | C | 35 | 25.680 | 51.764 | 80.262 | 1.00 | 39.07 | N |
| ATOM | 9571 | CE2 | TRP | C | 35 | 26.201 | 52.740 | 81.058 | 1.00 | 39.78 | C |
| ATOM | 9572 | CD2 | TRP | C | 35 | 27.353 | 52.211 | 81.662 | 1.00 | 37.48 | C |
| ATOM | 9573 | CE3 | TRP | C | 35 | 28.075 | 53.018 | 82.501 | 1.00 | 34.96 | C |
| ATOM | 9575 | CZ3 | TRP | C | 35 | 27.628 | 54.293 | 82.738 | 1.00 | 38.38 | C |
| ATOM | 9577 | CH2 | TRP | C | 35 | 26.484 | 54.789 | 82.138 | 1.00 | 41.25 | C |
| ATOM | 9579 | CZ2 | TRP | C | 35 | 25.744 | 54.022 | 81.306 | 1.00 | 42.46 | C |
| ATOM | 9581 | C | TRP | C | 35 | 30.803 | 49.082 | 81.568 | 1.00 | 34.40 | C |
| ATOM | 9582 | O | TRP | C | 35 | 30.787 | 47.871 | 81.643 | 1.00 | 34.68 | O |
| ATOM | 9583 | N | TYR | C | 36 | 31.619 | 49.809 | 82.295 | 1.00 | 34.73 | N |
| ATOM | 9585 | CA | TYR | C | 36 | 32.679 | 49.273 | 83.080 | 1.00 | 34.84 | C |
| ATOM | 9587 | CB | TYR | C | 36 | 33.979 | 49.790 | 82.457 | 1.00 | 33.85 | C |
| ATOM | 9590 | CG | TYR | C | 36 | 34.192 | 49.428 | 81.000 | 1.00 | 32.89 | C |
| ATOM | 9591 | CD1 | TYR | C | 36 | 33.730 | 50.226 | 79.935 | 1.00 | 34.08 | C |
| ATOM | 9593 | CE1 | TYR | C | 36 | 33.954 | 49.861 | 78.569 | 1.00 | 30.47 | C |
| ATOM | 9595 | CZ | TYR | C | 36 | 34.628 | 48.702 | 78.318 | 1.00 | 33.75 | C |
| ATOM | 9596 | OH | TYR | C | 36 | 34.972 | 48.211 | 77.099 | 1.00 | 32.78 | O |
| ATOM | 9598 | CE2 | TYR | C | 36 | 35.081 | 47.932 | 79.350 | 1.00 | 36.07 | C |
| ATOM | 9600 | CD2 | TYR | C | 36 | 34.855 | 48.298 | 80.683 | 1.00 | 34.48 | C |
| ATOM | 9602 | C | TYR | C | 36 | 32.638 | 49.720 | 84.560 | 1.00 | 36.53 | C |
| ATOM | 9603 | O | TYR | C | 36 | 32.184 | 50.821 | 84.911 | 1.00 | 37.45 | O |
| ATOM | 9604 | N | GLN | C | 37 | 33.181 | 48.875 | 85.422 | 1.00 | 37.01 | N |
| ATOM | 9606 | CA | GLN | C | 37 | 33.485 | 49.253 | 86.776 | 1.00 | 37.99 | C |
| ATOM | 9608 | CB | GLN | C | 37 | 32.866 | 48.245 | 87.662 | 1.00 | 40.20 | C |
| ATOM | 9611 | CG | GLN | C | 37 | 33.024 | 48.454 | 89.097 | 1.00 | 43.44 | C |
| ATOM | 9614 | CD | GLN | C | 37 | 32.484 | 47.260 | 89.850 | 1.00 | 44.50 | C |
| ATOM | 9615 | OE1 | GLN | C | 37 | 33.095 | 46.188 | 89.830 | 1.00 | 41.97 | O |
| ATOM | 9616 | NE2 | GLN | C | 37 | 31.341 | 47.450 | 90.519 | 1.00 | 45.99 | N |
| ATOM | 9619 | C | GLN | C | 37 | 34.971 | 49.202 | 86.950 | 1.00 | 38.04 | C |
| ATOM | 9620 | O | GLN | C | 37 | 35.576 | 48.347 | 86.420 | 1.00 | 37.13 | O |
| ATOM | 9621 | N | GLN | C | 38 | 35.532 | 50.155 | 87.691 | 1.00 | 39.43 | N |
| ATOM | 9623 | CA | GLN | C | 38 | 36.916 | 50.233 | 88.006 | 1.00 | 40.11 | C |
| ATOM | 9625 | CB | GLN | C | 38 | 37.572 | 51.356 | 87.212 | 1.00 | 40.00 | C |
| ATOM | 9628 | CG | GLN | C | 38 | 39.138 | 51.282 | 87.328 | 1.00 | 41.59 | C |
| ATOM | 9631 | CD | GLN | C | 38 | 39.903 | 52.189 | 86.386 | 1.00 | 39.71 | C |
| ATOM | 9632 | OE1 | GLN | C | 38 | 40.911 | 51.749 | 85.818 | 1.00 | 39.37 | O |
| ATOM | 9633 | NE2 | GLN | C | 38 | 39.488 | 53.456 | 86.280 | 1.00 | 36.17 | N |
| ATOM | 9636 | C | GLN | C | 38 | 37.025 | 50.536 | 89.490 | 1.00 | 42.44 | C |
| ATOM | 9637 | O | GLN | C | 38 | 36.535 | 51.554 | 89.948 | 1.00 | 43.21 | O |
| ATOM | 9638 | N | ARG | C | 39 | 37.659 | 49.658 | 90.248 | 1.00 | 43.81 | N |
| ATOM | 9640 | CA | ARG | C | 39 | 37.854 | 49.916 | 91.649 | 1.00 | 46.09 | C |
| ATOM | 9642 | CB | ARG | C | 39 | 37.775 | 48.654 | 92.479 | 1.00 | 47.38 | C |
| ATOM | 9645 | CG | ARG | C | 39 | 36.378 | 48.003 | 92.388 | 1.00 | 47.92 | C |
| ATOM | 9648 | CD | ARG | C | 39 | 36.268 | 46.636 | 93.005 | 1.00 | 49.95 | C |
| ATOM | 9651 | NE | ARG | C | 39 | 34.888 | 46.233 | 93.235 | 1.00 | 51.18 | N |
| ATOM | 9653 | CZ | ARG | C | 39 | 34.457 | 44.976 | 93.111 | 1.00 | 53.33 | C |
| ATOM | 9654 | NH1 | ARG | C | 39 | 35.280 | 44.025 | 92.746 | 1.00 | 55.36 | N |
| ATOM | 9657 | NH2 | ARG | C | 39 | 33.198 | 44.656 | 93.305 | 1.00 | 54.57 | N |
| ATOM | 9660 | C | ARG | C | 39 | 39.227 | 50.474 | 91.698 | 1.00 | 48.48 | C |
| ATOM | 9661 | O | ARG | C | 39 | 40.076 | 50.144 | 90.848 | 1.00 | 48.36 | O |
| ATOM | 9662 | N | THR | C | 40 | 39.446 | 51.325 | 92.696 | 1.00 | 50.49 | N |
| ATOM | 9664 | CA | THR | C | 40 | 40.767 | 51.785 | 93.054 | 1.00 | 52.37 | C |
| ATOM | 9666 | CB | THR | C | 40 | 40.743 | 52.104 | 94.505 | 1.00 | 54.92 | C |
| ATOM | 9668 | OG1 | THR | C | 40 | 39.665 | 53.023 | 94.716 | 1.00 | 55.56 | O |
| ATOM | 9670 | CG2 | THR | C | 40 | 41.930 | 52.878 | 94.904 | 1.00 | 57.62 | C |
| ATOM | 9674 | C | THR | C | 40 | 41.826 | 50.749 | 92.731 | 1.00 | 53.36 | C |
| ATOM | 9675 | O | THR | C | 40 | 41.665 | 49.544 | 93.025 | 1.00 | 54.89 | O |
| ATOM | 9676 | N | ASN | C | 41 | 42.870 | 51.206 | 92.053 | 1.00 | 52.76 | N |
| ATOM | 9678 | CA | ASN | C | 41 | 44.015 | 50.359 | 91.762 | 1.00 | 54.22 | C |
| ATOM | 9680 | CB | ASN | C | 41 | 44.688 | 49.980 | 93.075 | 1.00 | 58.44 | C |
| ATOM | 9683 | CG | ASN | C | 41 | 45.154 | 51.230 | 93.865 | 1.00 | 63.41 | C |
| ATOM | 9684 | OD1 | ASN | C | 41 | 45.183 | 52.360 | 93.337 | 1.00 | 62.65 | O |
| ATOM | 9685 | ND2 | ASN | C | 41 | 45.482 | 51.031 | 95.136 | 1.00 | 66.15 | N |
| ATOM | 9688 | C | ASN | C | 41 | 43.781 | 49.126 | 90.892 | 1.00 | 51.30 | C |
| ATOM | 9689 | O | ASN | C | 41 | 44.628 | 48.262 | 90.811 | 1.00 | 52.22 | O |
| ATOM | 9690 | N | GLY | C | 42 | 42.667 | 49.105 | 90.189 | 1.00 | 47.41 | N |
| ATOM | 9692 | CA | GLY | C | 42 | 42.301 | 47.978 | 89.359 | 1.00 | 46.02 | C |
| ATOM | 9695 | C | GLY | C | 42 | 42.014 | 48.314 | 87.911 | 1.00 | 43.20 | C |
| ATOM | 9696 | O | GLY | C | 42 | 41.863 | 49.478 | 87.503 | 1.00 | 41.61 | O |
| ATOM | 9697 | N | SER | C | 43 | 41.944 | 47.296 | 87.084 | 1.00 | 42.43 | N |
| ATOM | 9699 | CA | SER | C | 43 | 41.710 | 47.632 | 85.715 | 1.00 | 41.19 | C |
| ATOM | 9701 | CB | SER | C | 43 | 42.475 | 46.773 | 84.770 | 1.00 | 41.96 | C |
| ATOM | 9704 | OG | SER | C | 43 | 42.809 | 45.525 | 85.299 | 1.00 | 46.75 | O |
| ATOM | 9706 | C | SER | C | 43 | 40.215 | 47.594 | 85.505 | 1.00 | 39.79 | C |
| ATOM | 9707 | O | SER | C | 43 | 39.487 | 47.122 | 86.353 | 1.00 | 41.05 | O |
| ATOM | 9708 | N | PRO | C | 44 | 39.700 | 48.223 | 84.476 | 1.00 | 38.34 | N |
| ATOM | 9709 | CA | PRO | C | 44 | 38.252 | 48.202 | 84.306 | 1.00 | 37.29 | C |
| ATOM | 9711 | CB | PRO | C | 44 | 37.996 | 49.170 | 83.120 | 1.00 | 36.21 | C |
| ATOM | 9714 | CG | PRO | C | 44 | 39.228 | 49.834 | 82.904 | 1.00 | 37.50 | C |
| ATOM | 9717 | CD | PRO | C | 44 | 40.363 | 49.045 | 83.467 | 1.00 | 38.27 | C |
| ATOM | 9720 | C | PRO | C | 44 | 37.665 | 46.810 | 84.093 | 1.00 | 35.59 | C |
| ATOM | 9721 | O | PRO | C | 44 | 38.311 | 45.834 | 83.755 | 1.00 | 36.62 | O |
| ATOM | 9722 | N | ARG | C | 45 | 36.390 | 46.753 | 84.321 | 1.00 | 34.62 | N |
| ATOM | 9724 | CA | ARG | C | 45 | 35.656 | 45.514 | 84.232 | 1.00 | 35.68 | C |
| ATOM | 9726 | CB | ARG | C | 45 | 35.337 | 45.068 | 85.609 | 1.00 | 37.79 | C |
| ATOM | 9729 | CG | ARG | C | 45 | 34.555 | 43.888 | 85.738 | 1.00 | 38.23 | C |
| ATOM | 9732 | CD | ARG | C | 45 | 34.072 | 43.791 | 87.116 | 1.00 | 42.83 | C |
| ATOM | 9735 | NE | ARG | C | 45 | 33.417 | 42.512 | 87.398 | 1.00 | 49.92 | N |
| ATOM | 9737 | CZ | ARG | C | 45 | 32.649 | 42.276 | 88.464 | 1.00 | 48.72 | C |
| ATOM | 9738 | NH1 | ARG | C | 45 | 32.470 | 43.244 | 89.332 | 1.00 | 42.80 | N |
| ATOM | 9741 | NH2 | ARG | C | 45 | 32.061 | 41.070 | 88.622 | 1.00 | 53.29 | N |
| ATOM | 9744 | C | ARG | C | 45 | 34.371 | 45.749 | 83.473 | 1.00 | 34.81 | C |
| ATOM | 9745 | O | ARG | C | 45 | 33.579 | 46.617 | 83.770 | 1.00 | 34.72 | O |
| ATOM | 9746 | N | LEU | C | 46 | 34.174 | 44.950 | 82.464 | 1.00 | 35.29 | N |
| ATOM | 9748 | CA | LEU | C | 46 | 33.028 | 45.109 | 81.603 | 1.00 | 34.83 | C |
| ATOM | 9750 | CB | LEU | C | 46 | 33.294 | 44.349 | 80.299 | 1.00 | 34.21 | C |
| ATOM | 9753 | CG | LEU | C | 46 | 32.122 | 44.073 | 79.436 | 1.00 | 33.10 | C |
| ATOM | 9755 | CD1 | LEU | C | 46 | 31.697 | 45.418 | 78.882 | 1.00 | 34.82 | C |
| ATOM | 9759 | CD2 | LEU | C | 46 | 32.515 | 43.060 | 78.411 | 1.00 | 33.01 | C |
| ATOM | 9763 | C | LEU | C | 46 | 31.824 | 44.529 | 82.356 | 1.00 | 36.54 | C |
| ATOM | 9764 | O | LEU | C | 46 | 31.892 | 43.392 | 82.806 | 1.00 | 38.07 | O |
| ATOM | 9765 | N | LEU | C | 47 | 30.782 | 45.344 | 82.478 | 1.00 | 36.50 | N |
| ATOM | 9767 | CA | LEU | C | 47 | 29.501 | 45.034 | 83.164 | 1.00 | 39.21 | C |
| ATOM | 9769 | CB | LEU | C | 47 | 29.061 | 46.227 | 83.993 | 1.00 | 39.56 | C |
| ATOM | 9772 | CG | LEU | C | 47 | 30.000 | 46.623 | 85.085 | 1.00 | 39.66 | C |
| ATOM | 9774 | CD1 | LEU | C | 47 | 29.636 | 47.995 | 85.413 | 1.00 | 40.73 | C |
| ATOM | 9778 | CD2 | LEU | C | 47 | 29.808 | 45.699 | 86.273 | 1.00 | 43.21 | C |
| ATOM | 9782 | C | LEU | C | 47 | 28.293 | 44.773 | 82.237 | 1.00 | 39.13 | C |
| ATOM | 9783 | O | LEU | C | 47 | 27.510 | 43.838 | 82.443 | 1.00 | 41.74 | O |
| ATOM | 9784 | N | ILE | C | 48 | 28.172 | 45.648 | 81.243 | 1.00 | 36.93 | N |
| ATOM | 9786 | CA | ILE | C | 48 | 27.142 | 45.636 | 80.277 | 1.00 | 37.02 | C |
| ATOM | 9788 | CB | ILE | C | 48 | 26.072 | 46.575 | 80.758 | 1.00 | 38.96 | C |
| ATOM | 9790 | CG1 | ILE | C | 48 | 25.469 | 45.944 | 82.022 | 1.00 | 42.23 | C |
| ATOM | 9793 | CD1 | ILE | C | 48 | 24.088 | 45.977 | 82.163 | 1.00 | 43.44 | C |
| ATOM | 9797 | CG2 | ILE | C | 48 | 25.048 | 46.823 | 79.629 | 1.00 | 41.51 | C |
| ATOM | 9801 | C | ILE | C | 48 | 27.663 | 46.061 | 78.952 | 1.00 | 34.72 | C |
| ATOM | 9802 | O | ILE | C | 48 | 28.368 | 47.060 | 78.847 | 1.00 | 33.27 | O |
| ATOM | 9803 | N | LYS | C | 49 | 27.288 | 45.296 | 77.927 | 1.00 | 35.49 | N |
| ATOM | 9805 | CA | LYS | C | 49 | 27.566 | 45.597 | 76.514 | 1.00 | 33.86 | C |
| ATOM | 9807 | CB | LYS | C | 49 | 28.333 | 44.450 | 75.916 | 1.00 | 33.07 | C |
| ATOM | 9810 | CG | LYS | C | 49 | 27.661 | 43.106 | 76.022 | 1.00 | 38.02 | C |
| ATOM | 9813 | CD | LYS | C | 49 | 28.443 | 42.066 | 75.280 | 1.00 | 39.19 | C |
| ATOM | 9816 | CE | LYS | C | 49 | 27.679 | 41.455 | 74.125 | 1.00 | 40.58 | C |
| ATOM | 9819 | NZ | LYS | C | 49 | 28.697 | 40.846 | 73.223 | 1.00 | 41.24 | N |
| ATOM | 9823 | C | LYS | C | 49 | 26.249 | 45.889 | 75.720 | 1.00 | 34.77 | C |
| ATOM | 9824 | O | LYS | C | 49 | 25.183 | 45.350 | 75.981 | 1.00 | 35.85 | O |
| ATOM | 9825 | N | TYR | C | 50 | 26.335 | 46.780 | 74.771 | 1.00 | 33.83 | N |
| ATOM | 9827 | CA | TYR | C | 50 | 25.191 | 47.100 | 73.987 | 1.00 | 37.51 | C |
| ATOM | 9829 | CB | TYR | C | 50 | 24.878 | 46.016 | 72.959 | 1.00 | 38.19 | C |
| ATOM | 9832 | CG | TYR | C | 50 | 26.067 | 45.851 | 72.028 | 1.00 | 38.64 | C |
| ATOM | 9833 | CD1 | TYR | C | 50 | 26.984 | 44.826 | 72.216 | 1.00 | 37.12 | C |
| ATOM | 9835 | CE1 | TYR | C | 50 | 28.076 | 44.706 | 71.409 | 1.00 | 33.93 | C |
| ATOM | 9837 | CZ | TYR | C | 50 | 28.306 | 45.624 | 70.421 | 1.00 | 32.98 | C |
| ATOM | 9838 | OH | TYR | C | 50 | 29.410 | 45.521 | 69.616 | 1.00 | 34.60 | O |
| ATOM | 9840 | CE2 | TYR | C | 50 | 27.482 | 46.665 | 70.239 | 1.00 | 34.65 | C |
| ATOM | 9842 | CD2 | TYR | C | 50 | 26.363 | 46.797 | 71.037 | 1.00 | 38.98 | C |
| ATOM | 9844 | C | TYR | C | 50 | 24.014 | 47.451 | 74.872 | 1.00 | 40.75 | C |
| ATOM | 9845 | O | TYR | C | 50 | 22.900 | 46.896 | 74.766 | 1.00 | 42.82 | O |
| ATOM | 9846 | N | ALA | C | 51 | 24.282 | 48.432 | 75.747 | 1.00 | 40.74 | N |
| ATOM | 9848 | CA | ALA | C | 51 | 23.233 | 49.188 | 76.446 | 1.00 | 41.98 | C |
| ATOM | 9850 | CB | ALA | C | 51 | 22.286 | 49.720 | 75.487 | 1.00 | 42.55 | C |
| ATOM | 9854 | C | ALA | C | 51 | 22.556 | 48.363 | 77.536 | 1.00 | 43.53 | C |
| ATOM | 9855 | O | ALA | C | 51 | 22.440 | 48.796 | 78.673 | 1.00 | 43.80 | O |
| ATOM | 9856 | N | SER | C | 52 | 22.203 | 47.139 | 77.210 | 1.00 | 44.89 | N |
| ATOM | 9858 | CA | SER | C | 52 | 21.403 | 46.352 | 78.108 | 1.00 | 48.67 | C |
| ATOM | 9860 | CB | SER | C | 52 | 19.959 | 46.450 | 77.643 | 1.00 | 52.04 | C |
| ATOM | 9863 | OG | SER | C | 52 | 19.845 | 45.977 | 76.294 | 1.00 | 53.55 | O |
| ATOM | 9865 | C | SER | C | 52 | 21.783 | 44.880 | 78.181 | 1.00 | 48.92 | C |
| ATOM | 9866 | O | SER | C | 52 | 21.086 | 44.131 | 78.910 | 1.00 | 50.69 | O |
| ATOM | 9867 | N | GLU | C | 53 | 22.858 | 44.477 | 77.491 | 1.00 | 46.03 | N |
| ATOM | 9869 | CA | GLU | C | 53 | 23.166 | 43.060 | 77.423 | 1.00 | 47.67 | C |
| ATOM | 9871 | CB | GLU | C | 53 | 23.704 | 42.543 | 76.064 | 1.00 | 46.94 | C |
| ATOM | 9874 | CG | GLU | C | 53 | 22.938 | 42.960 | 74.829 | 1.00 | 49.55 | C |
| ATOM | 9877 | CD | GLU | C | 53 | 23.501 | 42.450 | 73.448 | 1.00 | 51.85 | C |
| ATOM | 9878 | OE1 | GLU | C | 53 | 24.687 | 41.939 | 73.270 | 1.00 | 43.43 | O |
| ATOM | 9879 | OE2 | GLU | C | 53 | 22.655 | 42.572 | 72.483 | 1.00 | 57.14 | O |
| ATOM | 9880 | C | GLU | C | 53 | 24.150 | 42.708 | 78.518 | 1.00 | 46.50 | C |
| ATOM | 9881 | O | GLU | C | 53 | 25.307 | 43.167 | 78.557 | 1.00 | 42.83 | O |
| ATOM | 9882 | N | SER | C | 54 | 23.689 | 41.775 | 79.342 | 1.00 | 49.02 | N |
| ATOM | 9884 | CA | SER | C | 54 | 24.458 | 41.304 | 80.479 | 1.00 | 49.21 | C |
| ATOM | 9886 | CB | SER | C | 54 | 23.589 | 40.467 | 81.346 | 1.00 | 52.35 | C |
| ATOM | 9889 | OG | SER | C | 54 | 23.295 | 39.351 | 80.585 | 1.00 | 56.61 | O |
| ATOM | 9891 | C | SER | C | 54 | 25.636 | 40.452 | 80.034 | 1.00 | 46.98 | C |
| ATOM | 9892 | O | SER | C | 54 | 25.625 | 39.895 | 78.944 | 1.00 | 45.39 | O |
| ATOM | 9893 | N | ILE | C | 55 | 26.611 | 40.392 | 80.941 | 1.00 | 45.65 | N |
| ATOM | 9895 | CA | ILE | C | 55 | 27.878 | 39.764 | 80.739 | 1.00 | 44.10 | C |
| ATOM | 9897 | CB | ILE | C | 55 | 28.910 | 40.806 | 80.912 | 1.00 | 40.90 | C |
| ATOM | 9899 | CG1 | ILE | C | 55 | 29.119 | 41.482 | 79.584 | 1.00 | 40.37 | C |
| ATOM | 9902 | CD1 | ILE | C | 55 | 29.086 | 42.819 | 79.727 | 1.00 | 42.31 | C |
| ATOM | 9906 | CG2 | ILE | C | 55 | 30.195 | 40.224 | 81.246 | 1.00 | 42.13 | C |
| ATOM | 9910 | C | ILE | C | 55 | 28.092 | 38.693 | 81.748 | 1.00 | 47.09 | C |
| ATOM | 9911 | O | ILE | C | 55 | 27.684 | 38.816 | 82.892 | 1.00 | 49.47 | O |
| ATOM | 9912 | N | SER | C | 56 | 28.746 | 37.611 | 81.384 | 1.00 | 48.71 | N |
| ATOM | 9914 | CA | SER | C | 56 | 28.702 | 36.538 | 82.343 | 1.00 | 52.52 | C |
| ATOM | 9916 | CB | SER | C | 56 | 28.994 | 35.174 | 81.716 | 1.00 | 54.74 | C |
| ATOM | 9919 | OG | SER | C | 56 | 30.317 | 34.847 | 82.071 | 1.00 | 57.29 | O |
| ATOM | 9921 | C | SER | C | 56 | 29.657 | 36.960 | 83.473 | 1.00 | 51.73 | C |
| ATOM | 9922 | O | SER | C | 56 | 30.536 | 37.820 | 83.277 | 1.00 | 48.86 | O |
| ATOM | 9923 | N | GLY | C | 57 | 29.442 | 36.398 | 84.659 | 1.00 | 54.10 | N |
| ATOM | 9925 | CA | GLY | C | 57 | 30.238 | 36.705 | 85.830 | 1.00 | 53.98 | C |
| ATOM | 9928 | C | GLY | C | 57 | 29.779 | 37.965 | 86.529 | 1.00 | 52.14 | C |
| ATOM | 9929 | O | GLY | C | 57 | 30.328 | 38.382 | 87.538 | 1.00 | 52.51 | O |
| ATOM | 9930 | N | ILE | C | 58 | 28.756 | 38.598 | 86.015 | 1.00 | 50.75 | N |
| ATOM | 9932 | CA | ILE | C | 58 | 28.314 | 39.794 | 86.658 | 1.00 | 49.91 | C |
| ATOM | 9934 | CB | ILE | C | 58 | 28.314 | 40.938 | 85.671 | 1.00 | 46.82 | C |
| ATOM | 9936 | CG1 | ILE | C | 58 | 29.765 | 41.294 | 85.421 | 1.00 | 45.06 | C |
| ATOM | 9939 | CD1 | ILE | C | 58 | 29.908 | 41.654 | 84.172 | 1.00 | 45.24 | C |
| ATOM | 9943 | CG2 | ILE | C | 58 | 27.517 | 42.240 | 86.215 | 1.00 | 45.62 | C |
| ATOM | 9947 | C | ILE | C | 58 | 26.990 | 39.664 | 87.360 | 1.00 | 53.05 | C |
| ATOM | 9948 | O | ILE | C | 58 | 25.977 | 39.199 | 86.799 | 1.00 | 53.28 | O |
| ATOM | 9949 | N | PRO | C | 59 | 27.025 | 40.124 | 88.607 | 1.00 | 54.73 | N |
| ATOM | 9950 | CA | PRO | C | 59 | 25.850 | 40.053 | 89.461 | 1.00 | 58.09 | C |
| ATOM | 9952 | CB | PRO | C | 59 | 26.262 | 40.797 | 90.748 | 1.00 | 58.25 | C |
| ATOM | 9955 | CG | PRO | C | 59 | 27.651 | 41.330 | 90.560 | 1.00 | 55.53 | C |
| ATOM | 9958 | CD | PRO | C | 59 | 28.183 | 40.749 | 89.287 | 1.00 | 53.29 | C |
| ATOM | 9961 | C | PRO | C | 59 | 24.629 | 40.652 | 88.749 | 1.00 | 57.82 | C |
| ATOM | 9962 | O | PRO | C | 59 | 24.644 | 41.654 | 88.035 | 1.00 | 53.30 | O |
| ATOM | 9963 | N | SER | C | 60 | 23.562 | 39.887 | 88.909 | 1.00 | 63.33 | N |
| ATOM | 9965 | CA | SER | C | 60 | 22.219 | 40.188 | 88.390 | 1.00 | 65.73 | C |
| ATOM | 9967 | CB | SER | C | 60 | 21.267 | 39.142 | 88.972 | 1.00 | 71.17 | C |
| ATOM | 9970 | OG | SER | C | 60 | 21.740 | 38.742 | 90.265 | 1.00 | 74.60 | O |
| ATOM | 9972 | C | SER | C | 60 | 21.779 | 41.655 | 88.744 | 1.00 | 64.64 | C |
| ATOM | 9973 | O | SER | C | 60 | 21.114 | 42.343 | 87.979 | 1.00 | 64.25 | O |
| ATOM | 9974 | N | ARG | C | 61 | 22.185 | 42.151 | 89.894 | 1.00 | 64.61 | N |
| ATOM | 9976 | CA | ARG | C | 61 | 21.829 | 43.513 | 90.261 | 1.00 | 63.48 | C |
| ATOM | 9978 | CB | ARG | C | 61 | 22.297 | 43.845 | 91.663 | 1.00 | 64.21 | C |
| ATOM | 9981 | CG | ARG | C | 61 | 23.414 | 43.056 | 92.181 | 1.00 | 63.18 | C |
| ATOM | 9984 | CD | ARG | C | 61 | 24.104 | 43.802 | 93.236 | 1.00 | 63.84 | C |
| ATOM | 9987 | NE | ARG | C | 61 | 25.537 | 43.714 | 93.099 | 1.00 | 61.19 | N |
| ATOM | 9989 | CZ | ARG | C | 61 | 26.362 | 43.963 | 94.043 | 1.00 | 58.51 | C |
| ATOM | 9990 | NH1 | ARG | C | 61 | 25.979 | 44.372 | 95.210 | 1.00 | 68.13 | N |
| ATOM | 9993 | NH2 | ARG | C | 61 | 27.590 | 43.835 | 93.832 | 1.00 | 58.31 | N |
| ATOM | 9996 | C | ARG | C | 61 | 22.316 | 44.585 | 89.309 | 1.00 | 59.40 | C |
| ATOM | 9997 | O | ARG | C | 61 | 21.757 | 45.689 | 89.288 | 1.00 | 60.37 | O |
| ATOM | 9998 | N | PHE | C | 62 | 23.370 | 44.295 | 88.562 | 1.00 | 55.68 | N |
| ATOM | 10000 | CA | PHE | C | 62 | 23.773 | 45.184 | 87.492 | 1.00 | 51.73 | C |
| ATOM | 10002 | CB | PHE | C | 62 | 25.226 | 44.908 | 87.114 | 1.00 | 49.74 | C |
| ATOM | 10005 | CG | PHE | C | 62 | 26.190 | 45.398 | 88.121 | 1.00 | 48.14 | C |
| ATOM | 10006 | CD1 | PHE | C | 62 | 26.773 | 44.542 | 89.009 | 1.00 | 49.83 | C |
| ATOM | 10008 | CE1 | PHE | C | 62 | 27.607 | 44.999 | 89.940 | 1.00 | 50.62 | C |
| ATOM | 10010 | CZ | PHE | C | 62 | 27.859 | 46.353 | 90.035 | 1.00 | 49.05 | C |
| ATOM | 10012 | CE2 | PHE | C | 62 | 27.252 | 47.208 | 89.173 | 1.00 | 46.83 | C |
| ATOM | 10014 | CD2 | PHE | C | 62 | 26.440 | 46.724 | 88.219 | 1.00 | 46.75 | C |
| ATOM | 10016 | C | PHE | C | 62 | 22.859 | 45.064 | 86.262 | 1.00 | 51.66 | C |
| ATOM | 10017 | O | PHE | C | 62 | 22.550 | 43.980 | 85.814 | 1.00 | 52.39 | O |
| ATOM | 10018 | N | SER | C | 63 | 22.447 | 46.200 | 85.726 | 1.00 | 50.26 | N |
| ATOM | 10020 | CA | SER | C | 63 | 21.681 | 46.249 | 84.502 | 1.00 | 51.03 | C |
| ATOM | 10022 | CB | SER | C | 63 | 20.242 | 45.917 | 84.784 | 1.00 | 55.13 | C |
| ATOM | 10025 | OG | SER | C | 63 | 19.490 | 47.066 | 85.148 | 1.00 | 57.69 | O |
| ATOM | 10027 | C | SER | C | 63 | 21.835 | 47.599 | 83.758 | 1.00 | 49.12 | C |
| ATOM | 10028 | O | SER | C | 63 | 22.366 | 48.536 | 84.265 | 1.00 | 47.72 | O |
| ATOM | 10029 | N | GLY | C | 64 | 21.405 | 47.648 | 82.513 | 1.00 | 49.99 | N |
| ATOM | 10031 | CA | GLY | C | 64 | 21.517 | 48.834 | 81.694 | 1.00 | 48.78 | C |
| ATOM | 10034 | C | GLY | C | 64 | 20.311 | 48.920 | 80.758 | 1.00 | 52.32 | C |
| ATOM | 10035 | O | GLY | C | 64 | 19.752 | 47.899 | 80.330 | 1.00 | 54.84 | O |
| ATOM | 10036 | N | SER | C | 65 | 19.880 | 50.152 | 80.489 | 1.00 | 53.63 | N |
| ATOM | 10038 | CA | SER | C | 65 | 18.816 | 50.477 | 79.546 | 1.00 | 56.59 | C |
| ATOM | 10040 | CB | SER | C | 65 | 17.528 | 50.804 | 80.288 | 1.00 | 61.01 | C |
| ATOM | 10043 | OG | SER | C | 65 | 17.844 | 51.826 | 81.211 | 1.00 | 63.48 | O |
| ATOM | 10045 | C | SER | C | 65 | 19.200 | 51.719 | 78.732 | 1.00 | 55.34 | C |
| ATOM | 10046 | O | SER | C | 65 | 20.157 | 52.439 | 79.066 | 1.00 | 53.24 | O |
| ATOM | 10047 | N | GLY | C | 66 | 18.429 | 51.969 | 77.660 | 1.00 | 57.45 | N |
| ATOM | 10049 | CA | GLY | C | 66 | 18.670 | 53.097 | 76.740 | 1.00 | 56.09 | C |
| ATOM | 10052 | C | GLY | C | 66 | 18.946 | 52.775 | 75.281 | 1.00 | 53.82 | C |
| ATOM | 10053 | O | GLY | C | 66 | 19.495 | 51.716 | 74.976 | 1.00 | 50.48 | O |
| ATOM | 10054 | N | SER | C | 67 | 18.490 | 53.659 | 74.404 | 1.00 | 55.01 | N |
| ATOM | 10056 | CA | SER | C | 67 | 18.997 | 53.730 | 73.072 | 1.00 | 55.14 | C |
| ATOM | 10058 | CB | SER | C | 67 | 18.520 | 52.591 | 72.105 | 1.00 | 56.95 | C |
| ATOM | 10061 | OG | SER | C | 67 | 17.150 | 52.540 | 71.891 | 1.00 | 61.53 | O |
| ATOM | 10063 | C | SER | C | 67 | 18.766 | 55.098 | 72.509 | 1.00 | 57.64 | C |
| ATOM | 10064 | O | SER | C | 67 | 17.849 | 55.760 | 72.887 | 1.00 | 61.04 | O |
| ATOM | 10065 | N | GLY | C | 68 | 19.654 | 55.501 | 71.597 | 1.00 | 57.28 | N |
| ATOM | 10067 | CA | GLY | C | 68 | 19.701 | 56.829 | 71.017 | 1.00 | 59.22 | C |
| ATOM | 10070 | C | GLY | C | 68 | 20.913 | 57.529 | 71.590 | 1.00 | 56.57 | C |
| ATOM | 10071 | O | GLY | C | 68 | 22.008 | 57.423 | 71.141 | 1.00 | 53.93 | O |
| ATOM | 10072 | N | THR | C | 69 | 20.681 | 58.136 | 72.712 | 1.00 | 58.48 | N |
| ATOM | 10074 | CA | THR | C | 69 | 21.398 | 59.306 | 73.137 | 1.00 | 59.19 | C |
| ATOM | 10076 | CB | THR | C | 69 | 20.645 | 60.517 | 72.548 | 1.00 | 64.50 | C |
| ATOM | 10078 | OG1 | THR | C | 69 | 21.537 | 61.132 | 71.613 | 1.00 | 67.14 | O |
| ATOM | 10080 | CG2 | THR | C | 69 | 20.230 | 61.612 | 73.564 | 1.00 | 66.00 | C |
| ATOM | 10084 | C | THR | C | 69 | 21.448 | 59.347 | 74.621 | 1.00 | 57.98 | C |
| ATOM | 10085 | O | THR | C | 69 | 22.437 | 59.751 | 75.159 | 1.00 | 55.59 | O |
| ATOM | 10086 | N | ASP | C | 70 | 20.375 | 58.909 | 75.272 | 1.00 | 60.07 | N |
| ATOM | 10088 | CA | ASP | C | 70 | 20.375 | 58.727 | 76.714 | 1.00 | 60.09 | C |
| ATOM | 10090 | CB | ASP | C | 70 | 19.133 | 59.425 | 77.247 | 1.00 | 64.32 | C |
| ATOM | 10093 | CG | ASP | C | 70 | 19.158 | 60.961 | 76.992 | 1.00 | 70.77 | C |
| ATOM | 10094 | OD1 | ASP | C | 70 | 20.277 | 61.595 | 77.042 | 1.00 | 67.90 | O |
| ATOM | 10095 | OD2 | ASP | C | 70 | 18.069 | 61.615 | 76.776 | 1.00 | 79.56 | O |
| ATOM | 10096 | C | ASP | C | 70 | 20.433 | 57.271 | 77.244 | 1.00 | 57.20 | C |
| ATOM | 10097 | O | ASP | C | 70 | 19.434 | 56.565 | 77.157 | 1.00 | 61.34 | O |
| ATOM | 10098 | N | PHE | C | 71 | 21.564 | 56.871 | 77.814 | 1.00 | 52.43 | N |
| ATOM | 10100 | CA | PHE | C | 71 | 21.775 | 55.575 | 78.451 | 1.00 | 51.08 | C |
| ATOM | 10102 | CB | PHE | C | 71 | 23.020 | 54.922 | 77.855 | 1.00 | 48.63 | C |
| ATOM | 10105 | CG | PHE | C | 71 | 23.070 | 55.028 | 76.388 | 1.00 | 47.77 | C |
| ATOM | 10106 | CD1 | PHE | C | 71 | 23.558 | 56.186 | 75.786 | 1.00 | 46.39 | C |
| ATOM | 10108 | CE1 | PHE | C | 71 | 23.531 | 56.351 | 74.450 | 1.00 | 47.37 | C |
| ATOM | 10110 | CZ | PHE | C | 71 | 23.001 | 55.340 | 73.654 | 1.00 | 50.56 | C |
| ATOM | 10112 | CE2 | PHE | C | 71 | 22.502 | 54.172 | 74.245 | 1.00 | 49.84 | C |
| ATOM | 10114 | CD2 | PHE | C | 71 | 22.531 | 54.039 | 75.620 | 1.00 | 48.19 | C |
| ATOM | 10116 | C | PHE | C | 71 | 21.950 | 55.645 | 79.988 | 1.00 | 50.97 | C |
| ATOM | 10117 | O | PHE | C | 71 | 22.106 | 56.674 | 80.535 | 1.00 | 52.40 | O |
| ATOM | 10118 | N | THR | C | 72 | 21.907 | 54.515 | 80.672 | 1.00 | 50.66 | N |
| ATOM | 10120 | CA | THR | C | 72 | 21.602 | 54.430 | 82.111 | 1.00 | 51.41 | C |
| ATOM | 10122 | CB | THR | C | 72 | 20.094 | 54.686 | 82.394 | 1.00 | 55.42 | C |
| ATOM | 10124 | OG1 | THR | C | 72 | 19.880 | 56.096 | 82.612 | 1.00 | 58.52 | O |
| ATOM | 10126 | CG2 | THR | C | 72 | 19.663 | 54.044 | 83.737 | 1.00 | 55.85 | C |
| ATOM | 10130 | C | THR | C | 72 | 22.009 | 53.029 | 82.632 | 1.00 | 49.89 | C |
| ATOM | 10131 | O | THR | C | 72 | 21.515 | 51.980 | 82.190 | 1.00 | 50.33 | O |
| ATOM | 10132 | N | LEU | C | 73 | 22.945 | 53.034 | 83.554 | 1.00 | 48.22 | N |
| ATOM | 10134 | CA | LEU | C | 73 | 23.410 | 51.850 | 84.229 | 1.00 | 47.33 | C |
| ATOM | 10136 | CB | LEU | C | 73 | 24.898 | 52.013 | 84.360 | 1.00 | 43.98 | C |
| ATOM | 10139 | CG | LEU | C | 73 | 25.579 | 50.891 | 85.084 | 1.00 | 43.79 | C |
| ATOM | 10141 | CD1 | LEU | C | 73 | 25.447 | 49.588 | 84.285 | 1.00 | 43.27 | C |
| ATOM | 10145 | CD2 | LEU | C | 73 | 27.020 | 51.257 | 85.309 | 1.00 | 39.29 | C |
| ATOM | 10149 | C | LEU | C | 73 | 22.760 | 51.821 | 85.616 | 1.00 | 50.10 | C |
| ATOM | 10150 | O | LEU | C | 73 | 22.792 | 52.823 | 86.325 | 1.00 | 51.66 | O |
| ATOM | 10151 | N | SER | C | 74 | 22.167 | 50.743 | 86.071 | 1.00 | 51.27 | N |
| ATOM | 10153 | CA | SER | C | 74 | 21.730 | 50.879 | 87.435 | 1.00 | 54.82 | C |
| ATOM | 10155 | CB | SER | C | 74 | 20.291 | 51.311 | 87.436 | 1.00 | 57.75 | C |
| ATOM | 10158 | OG | SER | C | 74 | 19.645 | 50.362 | 86.713 | 1.00 | 60.90 | O |
| ATOM | 10160 | C | SER | C | 74 | 21.976 | 49.663 | 88.313 | 1.00 | 56.29 | C |
| ATOM | 10161 | O | SER | C | 74 | 22.286 | 48.605 | 87.809 | 1.00 | 56.37 | O |
| ATOM | 10162 | N | ILE | C | 75 | 21.872 | 49.823 | 89.634 | 1.00 | 58.41 | N |
| ATOM | 10164 | CA | ILE | C | 75 | 22.106 | 48.720 | 90.561 | 1.00 | 59.78 | C |
| ATOM | 10166 | CB | ILE | C | 75 | 23.356 | 49.038 | 91.371 | 1.00 | 57.98 | C |
| ATOM | 10168 | CG1 | ILE | C | 75 | 24.460 | 49.483 | 90.438 | 1.00 | 54.60 | C |
| ATOM | 10171 | CD1 | ILE | C | 75 | 25.843 | 49.606 | 91.120 | 1.00 | 55.60 | C |
| ATOM | 10175 | CG2 | ILE | C | 75 | 23.802 | 47.821 | 92.132 | 1.00 | 59.77 | C |
| ATOM | 10179 | C | ILE | C | 75 | 20.937 | 48.621 | 91.491 | 1.00 | 64.00 | C |
| ATOM | 10180 | O | ILE | C | 75 | 20.583 | 49.616 | 92.011 | 1.00 | 65.00 | O |
| ATOM | 10181 | N | ASN | C | 76 | 20.325 | 47.458 | 91.705 | 1.00 | 67.66 | N |
| ATOM | 10183 | CA | ASN | C | 76 | 19.433 | 47.273 | 92.874 | 1.00 | 72.86 | C |
| ATOM | 10185 | CB | ASN | C | 76 | 18.573 | 46.066 | 92.666 | 1.00 | 77.07 | C |
| ATOM | 10188 | CG | ASN | C | 76 | 17.770 | 46.202 | 91.447 | 1.00 | 80.49 | C |
| ATOM | 10189 | OD1 | ASN | C | 76 | 17.965 | 47.202 | 90.704 | 1.00 | 80.41 | O |
| ATOM | 10190 | ND2 | ASN | C | 76 | 16.849 | 45.249 | 91.198 | 1.00 | 83.51 | N |
| ATOM | 10193 | C | ASN | C | 76 | 20.234 | 47.037 | 94.121 | 1.00 | 73.25 | C |
| ATOM | 10194 | O | ASN | C | 76 | 21.376 | 46.588 | 94.067 | 1.00 | 71.87 | O |
| ATOM | 10195 | N | SER | C | 77 | 19.686 | 47.302 | 95.282 | 1.00 | 75.89 | N |
| ATOM | 10197 | CA | SER | C | 77 | 20.510 | 47.059 | 96.480 | 1.00 | 76.19 | C |
| ATOM | 10199 | CB | SER | C | 77 | 20.179 | 45.692 | 97.115 | 1.00 | 80.08 | C |
| ATOM | 10202 | OG | SER | C | 77 | 21.236 | 44.803 | 96.770 | 1.00 | 81.82 | O |
| ATOM | 10204 | C | SER | C | 77 | 22.056 | 47.202 | 96.231 | 1.00 | 70.21 | C |
| ATOM | 10205 | O | SER | C | 77 | 22.780 | 46.223 | 95.964 | 1.00 | 66.16 | O |
| ATOM | 10206 | N | VAL | C | 78 | 22.515 | 48.446 | 96.376 | 1.00 | 68.55 | N |
| ATOM | 10208 | CA | VAL | C | 78 | 23.958 | 48.795 | 96.322 | 1.00 | 65.96 | C |
| ATOM | 10210 | CB | VAL | C | 78 | 24.263 | 50.293 | 96.464 | 1.00 | 64.03 | C |
| ATOM | 10212 | CG1 | VAL | C | 78 | 25.675 | 50.540 | 96.047 | 1.00 | 59.70 | C |
| ATOM | 10216 | CG2 | VAL | C | 78 | 23.367 | 51.101 | 95.666 | 1.00 | 63.10 | C |
| ATOM | 10220 | C | VAL | C | 78 | 24.733 | 48.220 | 97.475 | 1.00 | 68.31 | C |
| ATOM | 10221 | O | VAL | C | 78 | 24.269 | 48.259 | 98.604 | 1.00 | 70.79 | O |
| ATOM | 10222 | N | GLU | C | 79 | 25.939 | 47.756 | 97.184 | 1.00 | 67.29 | N |
| ATOM | 10224 | CA | GLU | C | 79 | 26.747 | 47.109 | 98.182 | 1.00 | 70.49 | C |
| ATOM | 10226 | CB | GLU | C | 79 | 27.015 | 45.646 | 97.772 | 1.00 | 71.59 | C |
| ATOM | 10229 | CG | GLU | C | 79 | 27.567 | 44.817 | 98.955 | 1.00 | 78.33 | C |
| ATOM | 10232 | CD | GLU | C | 79 | 27.217 | 43.318 | 98.941 | 1.00 | 81.18 | C |
| ATOM | 10233 | OE1 | GLU | C | 79 | 27.474 | 42.734 | 97.843 | 1.00 | 73.90 | O |
| ATOM | 10234 | OE2 | GLU | C | 79 | 26.760 | 42.776 | 100.055 | 1.00 | 81.77 | O |
| ATOM | 10235 | C | GLU | C | 79 | 28.063 | 47.841 | 98.381 | 1.00 | 69.03 | C |
| ATOM | 10236 | O | GLU | C | 79 | 28.525 | 48.598 | 97.500 | 1.00 | 66.31 | O |
| ATOM | 10237 | N | SER | C | 80 | 28.689 | 47.602 | 99.539 | 1.00 | 71.62 | N |
| ATOM | 10239 | CA | SER | C | 80 | 30.037 | 48.142 | 99.822 | 1.00 | 70.15 | C |
| ATOM | 10241 | CB | SER | C | 80 | 30.613 | 47.569 | 101.134 | 1.00 | 74.08 | C |
| ATOM | 10244 | OG | SER | C | 80 | 31.587 | 46.535 | 100.904 | 1.00 | 74.59 | O |
| ATOM | 10246 | C | SER | C | 80 | 30.987 | 47.854 | 98.682 | 1.00 | 66.56 | C |
| ATOM | 10247 | O | SER | C | 80 | 31.837 | 48.668 | 98.330 | 1.00 | 65.25 | O |
| ATOM | 10248 | N | GLU | C | 81 | 30.846 | 46.681 | 98.119 | 1.00 | 65.84 | N |
| ATOM | 10250 | CA | GLU | C | 81 | 31.744 | 46.272 | 97.084 | 1.00 | 64.00 | C |
| ATOM | 10252 | CB | GLU | C | 81 | 31.555 | 44.781 | 96.945 | 1.00 | 67.28 | C |
| ATOM | 10255 | CG | GLU | C | 81 | 32.772 | 43.977 | 96.520 | 1.00 | 70.30 | C |
| ATOM | 10258 | CD | GLU | C | 81 | 32.322 | 42.601 | 95.984 | 1.00 | 76.21 | C |
| ATOM | 10259 | OE1 | GLU | C | 81 | 31.393 | 41.987 | 96.673 | 1.00 | 77.86 | O |
| ATOM | 10260 | OE2 | GLU | C | 81 | 32.876 | 42.177 | 94.881 | 1.00 | 75.85 | O |
| ATOM | 10261 | C | GLU | C | 81 | 31.451 | 47.010 | 95.746 | 1.00 | 59.35 | C |
| ATOM | 10262 | O | GLU | C | 81 | 32.154 | 46.833 | 94.764 | 1.00 | 57.13 | O |
| ATOM | 10263 | N | ASP | C | 82 | 30.422 | 47.851 | 95.702 | 1.00 | 57.91 | N |
| ATOM | 10265 | CA | ASP | C | 82 | 30.121 | 48.600 | 94.488 | 1.00 | 54.01 | C |
| ATOM | 10267 | CB | ASP | C | 82 | 28.618 | 48.743 | 94.296 | 1.00 | 54.53 | C |
| ATOM | 10270 | CG | ASP | C | 82 | 27.901 | 47.367 | 94.085 | 1.00 | 57.28 | C |
| ATOM | 10271 | OD1 | ASP | C | 82 | 28.609 | 46.391 | 93.687 | 1.00 | 53.65 | O |
| ATOM | 10272 | OD2 | ASP | C | 82 | 26.642 | 47.201 | 94.289 | 1.00 | 58.60 | O |
| ATOM | 10273 | C | ASP | C | 82 | 30.766 | 49.950 | 94.511 | 1.00 | 52.01 | C |
| ATOM | 10274 | O | ASP | C | 82 | 30.626 | 50.689 | 93.606 | 1.00 | 49.44 | O |
| ATOM | 10275 | N | ILE | C | 83 | 31.480 | 50.285 | 95.562 | 1.00 | 53.49 | N |
| ATOM | 10277 | CA | ILE | C | 83 | 32.314 | 51.486 | 95.551 | 1.00 | 52.54 | C |
| ATOM | 10279 | CB | ILE | C | 83 | 32.877 | 51.678 | 96.961 | 1.00 | 54.86 | C |
| ATOM | 10281 | CG1 | ILE | C | 83 | 31.810 | 52.202 | 97.892 | 1.00 | 56.48 | C |
| ATOM | 10284 | CD1 | ILE | C | 83 | 32.220 | 52.011 | 99.307 | 1.00 | 59.82 | C |
| ATOM | 10288 | CG2 | ILE | C | 83 | 34.042 | 52.604 | 96.978 | 1.00 | 55.41 | C |
| ATOM | 10292 | C | ILE | C | 83 | 33.434 | 51.354 | 94.470 | 1.00 | 50.29 | C |
| ATOM | 10293 | O | ILE | C | 83 | 34.193 | 50.354 | 94.456 | 1.00 | 49.66 | O |
| ATOM | 10294 | N | ALA | C | 84 | 33.528 | 52.382 | 93.610 | 1.00 | 48.84 | N |
| ATOM | 10296 | CA | ALA | C | 84 | 34.175 | 52.304 | 92.263 | 1.00 | 46.44 | C |
| ATOM | 10298 | CB | ALA | C | 84 | 33.829 | 50.998 | 91.537 | 1.00 | 44.27 | C |
| ATOM | 10302 | C | ALA | C | 84 | 33.761 | 53.452 | 91.360 | 1.00 | 45.36 | C |
| ATOM | 10303 | O | ALA | C | 84 | 32.673 | 53.978 | 91.469 | 1.00 | 46.26 | O |
| ATOM | 10304 | N | ASP | C | 85 | 34.621 | 53.824 | 90.437 | 1.00 | 44.56 | N |
| ATOM | 10306 | CA | ASP | C | 85 | 34.175 | 54.588 | 89.275 | 1.00 | 43.73 | C |
| ATOM | 10308 | CB | ASP | C | 85 | 35.354 | 55.295 | 88.690 | 1.00 | 43.97 | C |
| ATOM | 10311 | CG | ASP | C | 85 | 35.925 | 56.290 | 89.604 | 1.00 | 47.86 | C |
| ATOM | 10312 | OD1 | ASP | C | 85 | 35.213 | 57.307 | 89.833 | 1.00 | 51.36 | O |
| ATOM | 10313 | OD2 | ASP | C | 85 | 37.066 | 56.130 | 90.098 | 1.00 | 46.79 | O |
| ATOM | 10314 | C | ASP | C | 85 | 33.502 | 53.735 | 88.135 | 1.00 | 41.60 | C |
| ATOM | 10315 | O | ASP | C | 85 | 33.984 | 52.694 | 87.751 | 1.00 | 40.63 | O |
| ATOM | 10316 | N | TYR | C | 86 | 32.394 | 54.211 | 87.600 | 1.00 | 40.84 | N |
| ATOM | 10318 | CA | TYR | C | 86 | 31.741 | 53.554 | 86.508 | 1.00 | 39.46 | C |
| ATOM | 10320 | CB | TYR | C | 86 | 30.278 | 53.370 | 86.862 | 1.00 | 40.82 | C |
| ATOM | 10323 | CG | TYR | C | 86 | 30.128 | 52.481 | 88.089 | 1.00 | 41.54 | C |
| ATOM | 10324 | CD1 | TYR | C | 86 | 30.275 | 52.972 | 89.390 | 1.00 | 40.87 | C |
| ATOM | 10326 | CE1 | TYR | C | 86 | 30.238 | 52.105 | 90.477 | 1.00 | 42.12 | C |
| ATOM | 10328 | CZ | TYR | C | 86 | 30.045 | 50.721 | 90.274 | 1.00 | 43.49 | C |
| ATOM | 10329 | OH | TYR | C | 86 | 29.967 | 49.762 | 91.277 | 1.00 | 44.20 | O |
| ATOM | 10331 | CE2 | TYR | C | 86 | 29.911 | 50.233 | 88.998 | 1.00 | 43.68 | C |
| ATOM | 10333 | CD2 | TYR | C | 86 | 29.947 | 51.111 | 87.922 | 1.00 | 43.03 | C |
| ATOM | 10335 | C | TYR | C | 86 | 31.981 | 54.442 | 85.282 | 1.00 | 39.22 | C |
| ATOM | 10336 | O | TYR | C | 86 | 31.996 | 55.699 | 85.428 | 1.00 | 39.82 | O |
| ATOM | 10337 | N | TYR | C | 87 | 32.248 | 53.781 | 84.121 | 1.00 | 37.23 | N |
| ATOM | 10339 | CA | TYR | C | 87 | 32.593 | 54.411 | 82.848 | 1.00 | 35.32 | C |
| ATOM | 10341 | CB | TYR | C | 87 | 34.068 | 54.178 | 82.513 | 1.00 | 34.25 | C |
| ATOM | 10344 | CG | TYR | C | 87 | 35.078 | 54.824 | 83.436 | 1.00 | 32.69 | C |
| ATOM | 10345 | CD1 | TYR | C | 87 | 35.673 | 54.085 | 84.435 | 1.00 | 34.43 | C |
| ATOM | 10347 | CE1 | TYR | C | 87 | 36.624 | 54.622 | 85.308 | 1.00 | 34.35 | C |
| ATOM | 10349 | CZ | TYR | C | 87 | 36.944 | 55.979 | 85.221 | 1.00 | 35.80 | C |
| ATOM | 10350 | OH | TYR | C | 87 | 37.860 | 56.518 | 86.145 | 1.00 | 36.98 | O |
| ATOM | 10352 | CE2 | TYR | C | 87 | 36.313 | 56.765 | 84.217 | 1.00 | 34.38 | C |
| ATOM | 10354 | CD2 | TYR | C | 87 | 35.427 | 56.154 | 83.317 | 1.00 | 32.88 | C |
| ATOM | 10356 | C | TYR | C | 87 | 31.659 | 53.868 | 81.712 | 1.00 | 35.54 | C |
| ATOM | 10357 | O | TYR | C | 87 | 31.079 | 52.769 | 81.798 | 1.00 | 33.51 | O |
| ATOM | 10358 | N | CYS | C | 88 | 31.451 | 54.688 | 80.674 | 1.00 | 36.35 | N |
| ATOM | 10360 | CA | CYS | C | 88 | 30.675 | 54.252 | 79.500 | 1.00 | 36.73 | C |
| ATOM | 10362 | CB | CYS | C | 88 | 29.397 | 55.021 | 79.326 | 1.00 | 38.36 | C |
| ATOM | 10365 | SG | CYS | C | 88 | 29.606 | 56.785 | 79.010 | 1.00 | 45.26 | S |
| ATOM | 10366 | C | CYS | C | 88 | 31.526 | 54.473 | 78.301 | 1.00 | 36.06 | C |
| ATOM | 10367 | O | CYS | C | 88 | 32.359 | 55.383 | 78.363 | 1.00 | 37.61 | O |
| ATOM | 10368 | N | GLN | C | 89 | 31.434 | 53.593 | 77.291 | 1.00 | 34.22 | N |
| ATOM | 10370 | CA | GLN | C | 89 | 32.123 | 53.844 | 76.047 | 1.00 | 35.12 | C |
| ATOM | 10372 | CB | GLN | C | 89 | 33.376 | 52.960 | 75.808 | 1.00 | 34.83 | C |
| ATOM | 10375 | CG | GLN | C | 89 | 33.011 | 51.589 | 75.387 | 1.00 | 35.80 | C |
| ATOM | 10378 | CD | GLN | C | 89 | 34.144 | 50.697 | 75.122 | 1.00 | 32.88 | C |
| ATOM | 10379 | OE1 | GLN | C | 89 | 35.021 | 50.995 | 74.338 | 1.00 | 28.59 | O |
| ATOM | 10380 | NE2 | GLN | C | 89 | 34.097 | 49.552 | 75.751 | 1.00 | 34.45 | N |
| ATOM | 10383 | C | GLN | C | 89 | 31.178 | 53.728 | 74.916 | 1.00 | 35.20 | C |
| ATOM | 10384 | O | GLN | C | 89 | 30.136 | 53.113 | 75.048 | 1.00 | 36.60 | O |
| ATOM | 10385 | N | GLN | C | 90 | 31.513 | 54.370 | 73.812 | 1.00 | 35.36 | N |
| ATOM | 10387 | CA | GLN | C | 90 | 30.724 | 54.222 | 72.614 | 1.00 | 35.88 | C |
| ATOM | 10389 | CB | GLN | C | 90 | 30.190 | 55.561 | 72.034 | 1.00 | 37.15 | C |
| ATOM | 10392 | CG | GLN | C | 90 | 31.160 | 56.518 | 71.509 | 1.00 | 38.90 | C |
| ATOM | 10395 | CD | GLN | C | 90 | 31.751 | 56.167 | 70.178 | 1.00 | 39.85 | C |
| ATOM | 10396 | OE1 | GLN | C | 90 | 31.045 | 55.686 | 69.291 | 1.00 | 39.79 | O |
| ATOM | 10397 | NE2 | GLN | C | 90 | 33.055 | 56.488 | 70.000 | 1.00 | 36.83 | N |
| ATOM | 10400 | C | GLN | C | 90 | 31.541 | 53.417 | 71.654 | 1.00 | 34.21 | C |
| ATOM | 10401 | O | GLN | C | 90 | 32.721 | 53.427 | 71.725 | 1.00 | 35.03 | O |
| ATOM | 10402 | N | ASN | C | 91 | 30.827 | 52.751 | 70.783 | 1.00 | 35.58 | N |
| ATOM | 10404 | CA | ASN | C | 91 | 31.209 | 51.680 | 69.873 | 1.00 | 35.22 | C |
| ATOM | 10406 | CB | ASN | C | 91 | 30.309 | 50.451 | 70.175 | 1.00 | 34.54 | C |
| ATOM | 10409 | CG | ASN | C | 91 | 31.052 | 49.282 | 69.987 | 1.00 | 34.93 | C |
| ATOM | 10410 | OD1 | ASN | C | 91 | 32.257 | 49.468 | 69.874 | 1.00 | 37.10 | O |
| ATOM | 10411 | ND2 | ASN | C | 91 | 30.452 | 48.094 | 69.806 | 1.00 | 34.50 | N |
| ATOM | 10414 | C | ASN | C | 91 | 30.932 | 52.048 | 68.387 | 1.00 | 36.89 | C |
| ATOM | 10415 | O | ASN | C | 91 | 31.327 | 51.330 | 67.449 | 1.00 | 35.22 | O |
| ATOM | 10416 | N | ASN | C | 92 | 30.203 | 53.164 | 68.223 | 1.00 | 37.82 | N |
| ATOM | 10418 | CA | ASN | C | 92 | 29.658 | 53.596 | 66.975 | 1.00 | 39.19 | C |
| ATOM | 10420 | CB | ASN | C | 92 | 28.456 | 54.437 | 67.328 | 1.00 | 42.61 | C |
| ATOM | 10423 | CG | ASN | C | 92 | 27.489 | 54.658 | 66.206 | 1.00 | 42.89 | C |
| ATOM | 10424 | OD1 | ASN | C | 92 | 27.520 | 54.082 | 65.292 | 1.00 | 44.19 | O |
| ATOM | 10425 | ND2 | ASN | C | 92 | 26.568 | 55.490 | 66.411 | 1.00 | 57.14 | N |
| ATOM | 10428 | C | ASN | C | 92 | 30.602 | 54.401 | 66.135 | 1.00 | 39.91 | C |
| ATOM | 10429 | O | ASN | C | 92 | 30.717 | 54.126 | 64.972 | 1.00 | 39.51 | O |
| ATOM | 10430 | N | ASN | C | 93 | 31.291 | 55.405 | 66.676 | 1.00 | 40.89 | N |
| ATOM | 10432 | CA | ASN | C | 93 | 32.127 | 56.280 | 65.792 | 1.00 | 43.39 | C |
| ATOM | 10434 | CB | ASN | C | 93 | 31.939 | 57.785 | 66.086 | 1.00 | 45.26 | C |
| ATOM | 10437 | CG | ASN | C | 93 | 30.611 | 58.254 | 65.777 | 1.00 | 45.20 | C |
| ATOM | 10438 | OD1 | ASN | C | 93 | 29.775 | 57.526 | 65.177 | 1.00 | 44.43 | O |
| ATOM | 10439 | ND2 | ASN | C | 93 | 30.318 | 59.439 | 66.265 | 1.00 | 49.86 | N |
| ATOM | 10442 | C | ASN | C | 93 | 33.573 | 55.924 | 65.885 | 1.00 | 42.51 | C |
| ATOM | 10443 | O | ASN | C | 93 | 33.867 | 55.305 | 66.862 | 1.00 | 43.58 | O |
| ATOM | 10444 | N | TRP | C | 94 | 34.457 | 56.343 | 64.958 | 1.00 | 43.96 | N |
| ATOM | 10446 | CA | TRP | C | 94 | 35.670 | 55.658 | 64.819 | 1.00 | 43.09 | C |
| ATOM | 10448 | CB | TRP | C | 94 | 36.318 | 55.596 | 63.431 | 1.00 | 45.10 | C |
| ATOM | 10451 | CG | TRP | C | 94 | 37.929 | 55.151 | 63.579 | 1.00 | 41.72 | C |
| ATOM | 10452 | CD1 | TRP | C | 94 | 38.440 | 53.861 | 63.794 | 1.00 | 37.65 | C |
| ATOM | 10454 | NE1 | TRP | C | 94 | 39.800 | 53.893 | 63.964 | 1.00 | 38.41 | N |
| ATOM | 10456 | CE2 | TRP | C | 94 | 40.213 | 55.197 | 63.962 | 1.00 | 41.29 | C |
| ATOM | 10457 | CD2 | TRP | C | 94 | 39.081 | 56.019 | 63.735 | 1.00 | 42.04 | C |
| ATOM | 10458 | CE3 | TRP | C | 94 | 39.264 | 57.397 | 63.732 | 1.00 | 44.37 | C |
| ATOM | 10460 | CZ3 | TRP | C | 94 | 40.504 | 57.884 | 63.875 | 1.00 | 46.24 | C |
| ATOM | 10462 | CH2 | TRP | C | 94 | 41.596 | 57.059 | 64.053 | 1.00 | 46.26 | C |
| ATOM | 10464 | CZ2 | TRP | C | 94 | 41.473 | 55.715 | 64.076 | 1.00 | 44.06 | C |
| ATOM | 10466 | C | TRP | C | 94 | 36.842 | 55.984 | 65.627 | 1.00 | 44.73 | C |
| ATOM | 10467 | O | TRP | C | 94 | 37.890 | 55.418 | 65.277 | 1.00 | 50.14 | O |
| ATOM | 10468 | N | PRO | C | 95 | 36.955 | 56.786 | 66.611 | 1.00 | 42.69 | N |
| ATOM | 10469 | CA | PRO | C | 95 | 37.708 | 56.193 | 67.739 | 1.00 | 39.74 | C |
| ATOM | 10471 | CB | PRO | C | 95 | 38.606 | 57.326 | 68.220 | 1.00 | 40.92 | C |
| ATOM | 10474 | CG | PRO | C | 95 | 38.406 | 58.340 | 67.337 | 1.00 | 43.30 | C |
| ATOM | 10477 | CD | PRO | C | 95 | 37.050 | 58.218 | 66.504 | 1.00 | 45.08 | C |
| ATOM | 10480 | C | PRO | C | 95 | 36.737 | 55.730 | 68.741 | 1.00 | 37.51 | C |
| ATOM | 10481 | O | PRO | C | 95 | 35.826 | 56.513 | 68.897 | 1.00 | 40.73 | O |
| ATOM | 10482 | N | THR | C | 96 | 36.836 | 54.564 | 69.340 | 1.00 | 35.57 | N |
| ATOM | 10484 | CA | THR | C | 96 | 36.133 | 54.302 | 70.597 | 1.00 | 35.67 | C |
| ATOM | 10486 | CB | THR | C | 96 | 36.506 | 52.995 | 71.300 | 1.00 | 34.47 | C |
| ATOM | 10488 | OG1 | THR | C | 96 | 36.874 | 52.031 | 70.376 | 1.00 | 40.36 | O |
| ATOM | 10490 | CG2 | THR | C | 96 | 35.298 | 52.345 | 71.897 | 1.00 | 34.45 | C |
| ATOM | 10494 | C | THR | C | 96 | 36.545 | 55.342 | 71.633 | 1.00 | 37.15 | C |
| ATOM | 10495 | O | THR | C | 96 | 37.697 | 55.771 | 71.699 | 1.00 | 38.19 | O |
| ATOM | 10496 | N | THR | C | 97 | 35.597 | 55.641 | 72.520 | 1.00 | 37.17 | N |
| ATOM | 10498 | CA | THR | C | 97 | 35.688 | 56.759 | 73.381 | 1.00 | 37.73 | C |
| ATOM | 10500 | CB | THR | C | 97 | 34.989 | 57.870 | 72.675 | 1.00 | 39.59 | C |
| ATOM | 10502 | OG1 | THR | C | 97 | 35.841 | 58.260 | 71.625 | 1.00 | 39.41 | O |
| ATOM | 10504 | CG2 | THR | C | 97 | 34.957 | 59.110 | 73.454 | 1.00 | 45.48 | C |
| ATOM | 10508 | C | THR | C | 97 | 35.031 | 56.368 | 74.658 | 1.00 | 36.93 | C |
| ATOM | 10509 | O | THR | C | 97 | 34.132 | 55.590 | 74.698 | 1.00 | 36.08 | O |
| ATOM | 10510 | N | PHE | C | 98 | 35.522 | 56.933 | 75.731 | 1.00 | 38.65 | N |
| ATOM | 10512 | CA | PHE | C | 98 | 34.986 | 56.695 | 77.041 | 1.00 | 37.72 | C |
| ATOM | 10514 | CB | PHE | C | 98 | 36.159 | 56.124 | 77.902 | 1.00 | 37.47 | C |
| ATOM | 10517 | CG | PHE | C | 98 | 36.606 | 54.763 | 77.485 | 1.00 | 34.26 | C |
| ATOM | 10518 | CD1 | PHE | C | 98 | 37.578 | 54.609 | 76.544 | 1.00 | 35.84 | C |
| ATOM | 10520 | CE1 | PHE | C | 98 | 38.002 | 53.331 | 76.187 | 1.00 | 34.75 | C |
| ATOM | 10522 | CZ | PHE | C | 98 | 37.421 | 52.229 | 76.743 | 1.00 | 29.78 | C |
| ATOM | 10524 | CE2 | PHE | C | 98 | 36.491 | 52.389 | 77.668 | 1.00 | 29.34 | C |
| ATOM | 10526 | CD2 | PHE | C | 98 | 36.090 | 53.645 | 78.051 | 1.00 | 31.59 | C |
| ATOM | 10528 | C | PHE | C | 98 | 34.422 | 58.060 | 77.591 | 1.00 | 39.16 | C |
| ATOM | 10529 | O | PHE | C | 98 | 34.737 | 59.159 | 77.062 | 1.00 | 40.05 | O |
| ATOM | 10530 | N | GLY | C | 99 | 33.598 | 57.951 | 78.645 | 1.00 | 38.61 | N |
| ATOM | 10532 | CA | GLY | C | 99 | 33.116 | 59.091 | 79.423 | 1.00 | 40.58 | C |
| ATOM | 10535 | C | GLY | C | 99 | 34.047 | 59.347 | 80.600 | 1.00 | 40.69 | C |
| ATOM | 10536 | O | GLY | C | 99 | 34.881 | 58.511 | 80.838 | 1.00 | 39.99 | O |
| ATOM | 10537 | N | ALA | C | 100 | 33.936 | 60.467 | 81.318 | 1.00 | 42.29 | N |
| ATOM | 10539 | CA | ALA | C | 100 | 34.929 | 60.800 | 82.331 | 1.00 | 43.08 | C |
| ATOM | 10541 | CB | ALA | C | 100 | 34.952 | 62.200 | 82.593 | 1.00 | 45.44 | C |
| ATOM | 10545 | C | ALA | C | 100 | 34.704 | 60.073 | 83.615 | 1.00 | 43.15 | C |
| ATOM | 10546 | O | ALA | C | 100 | 35.530 | 60.158 | 84.533 | 1.00 | 46.01 | O |
| ATOM | 10547 | N | GLY | C | 101 | 33.582 | 59.386 | 83.698 | 1.00 | 42.22 | N |
| ATOM | 10549 | CA | GLY | C | 101 | 33.303 | 58.487 | 84.799 | 1.00 | 41.44 | C |
| ATOM | 10552 | C | GLY | C | 101 | 32.429 | 59.141 | 85.833 | 1.00 | 42.90 | C |
| ATOM | 10553 | O | GLY | C | 101 | 32.196 | 60.338 | 85.710 | 1.00 | 43.82 | O |
| ATOM | 10554 | N | THR | C | 102 | 31.927 | 58.324 | 86.776 | 1.00 | 42.89 | N |
| ATOM | 10556 | CA | THR | C | 102 | 31.102 | 58.722 | 87.925 | 1.00 | 44.84 | C |
| ATOM | 10558 | CB | THR | C | 102 | 29.615 | 58.489 | 87.561 | 1.00 | 45.99 | C |
| ATOM | 10560 | OG1 | THR | C | 102 | 29.167 | 59.579 | 86.758 | 1.00 | 48.49 | O |
| ATOM | 10562 | CG2 | THR | C | 102 | 28.616 | 58.548 | 88.759 | 1.00 | 48.28 | C |
| ATOM | 10566 | C | THR | C | 102 | 31.537 | 57.838 | 89.083 | 1.00 | 44.23 | C |
| ATOM | 10567 | O | THR | C | 102 | 31.749 | 56.657 | 88.885 | 1.00 | 43.22 | O |
| ATOM | 10568 | N | LYS | C | 103 | 31.688 | 58.393 | 90.278 | 1.00 | 45.21 | N |
| ATOM | 10570 | CA | LYS | C | 103 | 32.237 | 57.643 | 91.412 | 1.00 | 45.48 | C |
| ATOM | 10572 | CB | LYS | C | 103 | 33.267 | 58.475 | 92.288 | 1.00 | 47.73 | C |
| ATOM | 10575 | CG | LYS | C | 103 | 34.082 | 57.608 | 93.362 | 1.00 | 48.87 | C |
| ATOM | 10578 | CD | LYS | C | 103 | 35.298 | 58.255 | 93.982 | 1.00 | 53.95 | C |
| ATOM | 10581 | CE | LYS | C | 103 | 35.376 | 58.007 | 95.634 | 1.00 | 64.04 | C |
| ATOM | 10584 | NZ | LYS | C | 103 | 34.907 | 59.016 | 96.915 | 1.00 | 61.58 | N |
| ATOM | 10588 | C | LYS | C | 103 | 31.069 | 57.287 | 92.237 | 1.00 | 46.12 | C |
| ATOM | 10589 | O | LYS | C | 103 | 30.196 | 58.112 | 92.468 | 1.00 | 48.00 | O |
| ATOM | 10590 | N | LEU | C | 104 | 31.042 | 56.098 | 92.776 | 1.00 | 46.17 | N |
| ATOM | 10592 | CA | LEU | C | 104 | 29.904 | 55.784 | 93.608 | 1.00 | 49.29 | C |
| ATOM | 10594 | CB | LEU | C | 104 | 29.523 | 54.342 | 93.380 | 1.00 | 49.15 | C |
| ATOM | 10597 | CG | LEU | C | 104 | 28.282 | 53.863 | 94.082 | 1.00 | 53.47 | C |
| ATOM | 10599 | CD1 | LEU | C | 104 | 27.166 | 54.601 | 93.392 | 1.00 | 56.73 | C |
| ATOM | 10603 | CD2 | LEU | C | 104 | 28.068 | 52.313 | 94.003 | 1.00 | 54.16 | C |
| ATOM | 10607 | C | LEU | C | 104 | 30.351 | 55.941 | 94.993 | 1.00 | 51.44 | C |
| ATOM | 10608 | O | LEU | C | 104 | 31.362 | 55.356 | 95.326 | 1.00 | 53.07 | O |
| ATOM | 10609 | N | GLU | C | 105 | 29.662 | 56.720 | 95.796 | 1.00 | 53.36 | N |
| ATOM | 10611 | CA | GLU | C | 105 | 29.914 | 56.710 | 97.228 | 1.00 | 56.53 | C |
| ATOM | 10613 | CB | GLU | C | 105 | 30.270 | 58.104 | 97.697 | 1.00 | 58.68 | C |
| ATOM | 10616 | CG | GLU | C | 105 | 30.158 | 58.333 | 99.212 | 1.00 | 63.77 | C |
| ATOM | 10619 | CD | GLU | C | 105 | 30.265 | 59.803 | 99.573 | 1.00 | 68.90 | C |
| ATOM | 10620 | OE1 | GLU | C | 105 | 29.358 | 60.563 | 99.176 | 1.00 | 72.30 | O |
| ATOM | 10621 | OE2 | GLU | C | 105 | 31.249 | 60.206 | 100.245 | 1.00 | 72.61 | O |
| ATOM | 10622 | C | GLU | C | 105 | 28.705 | 56.246 | 98.031 | 1.00 | 58.76 | C |
| ATOM | 10623 | O | GLU | C | 105 | 27.599 | 56.744 | 97.824 | 1.00 | 59.09 | O |
| ATOM | 10624 | N | LEU | C | 106 | 28.930 | 55.327 | 98.977 | 1.00 | 60.31 | N |
| ATOM | 10626 | CA | LEU | C | 106 | 27.904 | 54.929 | 99.963 | 1.00 | 62.77 | C |
| ATOM | 10628 | CB | LEU | C | 106 | 28.333 | 53.678 | 100.722 | 1.00 | 64.45 | C |
| ATOM | 10631 | CG | LEU | C | 106 | 28.259 | 52.375 | 99.937 | 1.00 | 64.30 | C |
| ATOM | 10633 | CD1 | LEU | C | 106 | 28.586 | 51.154 | 100.833 | 1.00 | 67.13 | C |
| ATOM | 10637 | CD2 | LEU | C | 106 | 26.847 | 52.259 | 99.368 | 1.00 | 66.90 | C |
| ATOM | 10641 | C | LEU | C | 106 | 27.715 | 55.987 | 100.994 | 1.00 | 64.27 | C |
| ATOM | 10642 | O | LEU | C | 106 | 28.692 | 56.430 | 101.558 | 1.00 | 63.86 | O |
| ATOM | 10643 | N | LYS | C | 107 | 26.474 | 56.364 | 101.274 | 1.00 | 65.47 | N |
| ATOM | 10645 | CA | LYS | C | 107 | 26.248 | 57.151 | 102.447 | 1.00 | 68.21 | C |
| ATOM | 10647 | CB | LYS | C | 107 | 25.055 | 58.121 | 102.387 | 1.00 | 70.02 | C |
| ATOM | 10650 | CG | LYS | C | 107 | 24.467 | 58.503 | 101.031 | 1.00 | 69.45 | C |
| ATOM | 10653 | CD | LYS | C | 107 | 23.340 | 59.617 | 101.174 | 1.00 | 75.00 | C |
| ATOM | 10656 | CE | LYS | C | 107 | 21.839 | 59.052 | 101.327 | 1.00 | 80.27 | C |
| ATOM | 10659 | NZ | LYS | C | 107 | 21.257 | 58.305 | 100.105 | 1.00 | 78.29 | N |
| ATOM | 10663 | C | LYS | C | 107 | 26.191 | 56.193 | 103.612 | 1.00 | 70.40 | C |
| ATOM | 10664 | O | LYS | C | 107 | 26.242 | 54.991 | 103.448 | 1.00 | 69.16 | O |
| ATOM | 10665 | N | ARG | C | 108 | 26.094 | 56.784 | 104.795 | 1.00 | 55.67 | N |
| ATOM | 10667 | CA | ARG | C | 108 | 26.552 | 56.234 | 106.070 | 1.00 | 55.57 | C |
| ATOM | 10669 | CB | ARG | C | 108 | 28.060 | 56.047 | 105.949 | 1.00 | 55.68 | C |
| ATOM | 10672 | CG | ARG | C | 108 | 28.725 | 55.336 | 107.046 | 1.00 | 56.83 | C |
| ATOM | 10675 | CD | ARG | C | 108 | 29.579 | 56.238 | 107.866 | 1.00 | 57.77 | C |
| ATOM | 10678 | NE | ARG | C | 108 | 29.552 | 55.864 | 109.288 | 1.00 | 57.84 | N |
| ATOM | 10680 | CZ | ARG | C | 108 | 30.316 | 54.919 | 109.761 | 1.00 | 60.59 | C |
| ATOM | 10681 | NH1 | ARG | C | 108 | 31.146 | 54.248 | 108.965 | 1.00 | 62.42 | N |
| ATOM | 10684 | NH2 | ARG | C | 108 | 30.258 | 54.623 | 111.020 | 1.00 | 64.86 | N |
| ATOM | 10687 | C | ARG | C | 108 | 26.229 | 57.307 | 107.130 | 1.00 | 54.10 | C |
| ATOM | 10688 | O | ARG | C | 108 | 26.110 | 58.500 | 106.783 | 1.00 | 52.40 | O |
| ATOM | 10689 | N | THR | C | 109 | 26.067 | 56.911 | 108.401 | 1.00 | 55.56 | N |
| ATOM | 10691 | CA | THR | C | 109 | 25.907 | 57.934 | 109.458 | 1.00 | 56.08 | C |
| ATOM | 10693 | CB | THR | C | 109 | 25.537 | 57.387 | 110.847 | 1.00 | 58.53 | C |
| ATOM | 10695 | OG1 | THR | C | 109 | 26.528 | 56.464 | 111.278 | 1.00 | 58.94 | O |
| ATOM | 10697 | CG2 | THR | C | 109 | 24.244 | 56.654 | 110.836 | 1.00 | 60.67 | C |
| ATOM | 10701 | C | THR | C | 109 | 27.167 | 58.773 | 109.655 | 1.00 | 54.85 | C |
| ATOM | 10702 | O | THR | C | 109 | 28.294 | 58.337 | 109.480 | 1.00 | 54.01 | O |
| ATOM | 10703 | N | VAL | C | 110 | 26.945 | 59.997 | 110.058 | 1.00 | 55.32 | N |
| ATOM | 10705 | CA | VAL | C | 110 | 28.021 | 60.900 | 110.341 | 1.00 | 55.05 | C |
| ATOM | 10707 | CB | VAL | C | 110 | 27.434 | 62.253 | 110.890 | 1.00 | 56.58 | C |
| ATOM | 10709 | CG1 | VAL | C | 110 | 28.528 | 63.180 | 111.294 | 1.00 | 57.91 | C |
| ATOM | 10713 | CG2 | VAL | C | 110 | 26.507 | 62.970 | 109.873 | 1.00 | 55.27 | C |
| ATOM | 10717 | C | VAL | C | 110 | 28.999 | 60.194 | 111.343 | 1.00 | 56.38 | C |
| ATOM | 10718 | O | VAL | C | 110 | 28.608 | 59.690 | 112.392 | 1.00 | 57.15 | O |
| ATOM | 10719 | N | ALA | C | 111 | 30.277 | 60.109 | 110.979 | 1.00 | 56.38 | N |
| ATOM | 10721 | CA | ALA | C | 111 | 31.305 | 59.519 | 111.847 | 1.00 | 57.80 | C |
| ATOM | 10723 | CB | ALA | C | 111 | 31.851 | 58.283 | 111.228 | 1.00 | 57.81 | C |
| ATOM | 10727 | C | ALA | C | 111 | 32.433 | 60.527 | 112.064 | 1.00 | 57.43 | C |
| ATOM | 10728 | O | ALA | C | 111 | 33.114 | 60.930 | 111.134 | 1.00 | 55.47 | O |
| ATOM | 10729 | N | ALA | C | 112 | 32.619 | 60.918 | 113.317 | 1.00 | 59.73 | N |
| ATOM | 10731 | CA | ALA | C | 112 | 33.580 | 61.947 | 113.670 | 1.00 | 59.71 | C |
| ATOM | 10733 | CB | ALA | C | 112 | 33.324 | 62.564 | 115.064 | 1.00 | 61.97 | C |
| ATOM | 10737 | C | ALA | C | 112 | 34.938 | 61.327 | 113.593 | 1.00 | 59.10 | C |
| ATOM | 10738 | O | ALA | C | 112 | 35.109 | 60.135 | 113.881 | 1.00 | 60.02 | O |
| ATOM | 10739 | N | PRO | C | 113 | 35.869 | 62.147 | 113.125 | 1.00 | 57.92 | N |
| ATOM | 10740 | CA | PRO | C | 113 | 37.241 | 61.752 | 112.972 | 1.00 | 57.27 | C |
| ATOM | 10742 | CB | PRO | C | 113 | 37.830 | 62.929 | 112.166 | 1.00 | 56.41 | C |
| ATOM | 10745 | CG | PRO | C | 113 | 37.022 | 64.097 | 112.509 | 1.00 | 56.68 | C |
| ATOM | 10748 | CD | PRO | C | 113 | 35.676 | 63.541 | 112.663 | 1.00 | 57.56 | C |
| ATOM | 10751 | C | PRO | C | 113 | 37.884 | 61.697 | 114.306 | 1.00 | 59.38 | C |
| ATOM | 10752 | O | PRO | C | 113 | 37.599 | 62.541 | 115.106 | 1.00 | 61.02 | O |
| ATOM | 10753 | N | SER | C | 114 | 38.761 | 60.740 | 114.498 | 1.00 | 59.86 | N |
| ATOM | 10755 | CA | SER | C | 114 | 39.630 | 60.699 | 115.606 | 1.00 | 62.67 | C |
| ATOM | 10757 | CB | SER | C | 114 | 39.923 | 59.236 | 115.876 | 1.00 | 64.64 | C |
| ATOM | 10760 | OG | SER | C | 114 | 38.718 | 58.528 | 116.164 | 1.00 | 66.40 | O |
| ATOM | 10762 | C | SER | C | 114 | 40.876 | 61.455 | 115.164 | 1.00 | 62.27 | C |
| ATOM | 10763 | O | SER | C | 114 | 41.536 | 61.049 | 114.215 | 1.00 | 61.52 | O |
| ATOM | 10764 | N | VAL | C | 115 | 41.212 | 62.536 | 115.849 | 1.00 | 64.21 | N |
| ATOM | 10766 | CA | VAL | C | 115 | 42.412 | 63.338 | 115.568 | 1.00 | 64.84 | C |
| ATOM | 10768 | CB | VAL | C | 115 | 42.112 | 64.867 | 115.740 | 1.00 | 66.16 | C |
| ATOM | 10770 | CG1 | VAL | C | 115 | 43.187 | 65.745 | 115.105 | 1.00 | 65.30 | C |
| ATOM | 10774 | CG2 | VAL | C | 115 | 40.751 | 65.225 | 115.112 | 1.00 | 65.65 | C |
| ATOM | 10778 | C | VAL | C | 115 | 43.656 | 62.979 | 116.413 | 1.00 | 67.80 | C |
| ATOM | 10779 | O | VAL | C | 115 | 43.555 | 62.609 | 117.547 | 1.00 | 71.74 | O |
| ATOM | 10780 | N | PHE | C | 116 | 44.834 | 63.156 | 115.822 | 1.00 | 67.72 | N |
| ATOM | 10782 | CA | PHE | C | 116 | 46.130 | 62.702 | 116.329 | 1.00 | 69.88 | C |
| ATOM | 10784 | CB | PHE | C | 116 | 46.381 | 61.247 | 115.944 | 1.00 | 69.43 | C |
| ATOM | 10787 | CG | PHE | C | 116 | 45.304 | 60.316 | 116.387 | 1.00 | 69.98 | C |
| ATOM | 10788 | CD1 | PHE | C | 116 | 44.217 | 60.065 | 115.575 | 1.00 | 66.04 | C |
| ATOM | 10790 | CE1 | PHE | C | 116 | 43.240 | 59.211 | 115.989 | 1.00 | 68.00 | C |
| ATOM | 10792 | CZ | PHE | C | 116 | 43.309 | 58.597 | 117.221 | 1.00 | 71.83 | C |
| ATOM | 10794 | CE2 | PHE | C | 116 | 44.367 | 58.840 | 118.040 | 1.00 | 75.85 | C |
| ATOM | 10796 | CD2 | PHE | C | 116 | 45.367 | 59.710 | 117.630 | 1.00 | 74.09 | C |
| ATOM | 10798 | C | PHE | C | 116 | 47.217 | 63.542 | 115.687 | 1.00 | 69.39 | C |
| ATOM | 10799 | O | PHE | C | 116 | 47.290 | 63.629 | 114.457 | 1.00 | 67.03 | O |
| ATOM | 10800 | N | ILE | C | 117 | 48.055 | 64.175 | 116.502 | 1.00 | 72.96 | N |
| ATOM | 10802 | CA | ILE | C | 117 | 49.120 | 65.045 | 115.980 | 1.00 | 72.96 | C |
| ATOM | 10804 | CB | ILE | C | 117 | 49.000 | 66.482 | 116.565 | 1.00 | 74.87 | C |
| ATOM | 10806 | CG1 | ILE | C | 117 | 50.181 | 67.350 | 116.111 | 1.00 | 76.75 | C |
| ATOM | 10809 | CD1 | ILE | C | 117 | 49.855 | 68.819 | 115.887 | 1.00 | 77.88 | C |
| ATOM | 10813 | CG2 | ILE | C | 117 | 48.837 | 66.451 | 118.062 | 1.00 | 79.54 | C |
| ATOM | 10817 | C | ILE | C | 117 | 50.458 | 64.376 | 116.247 | 1.00 | 75.27 | C |
| ATOM | 10818 | O | ILE | C | 117 | 50.557 | 63.617 | 117.210 | 1.00 | 79.55 | O |
| ATOM | 10819 | N | PHE | C | 118 | 51.457 | 64.596 | 115.387 | 1.00 | 74.30 | N |
| ATOM | 10821 | CA | PHE | C | 118 | 52.774 | 63.940 | 115.508 | 1.00 | 76.66 | C |
| ATOM | 10823 | CB | PHE | C | 118 | 52.929 | 62.877 | 114.426 | 1.00 | 74.55 | C |
| ATOM | 10826 | CG | PHE | C | 118 | 51.971 | 61.709 | 114.544 | 1.00 | 74.34 | C |
| ATOM | 10827 | CD1 | PHE | C | 118 | 50.831 | 61.640 | 113.735 | 1.00 | 73.64 | C |
| ATOM | 10829 | CE1 | PHE | C | 118 | 49.942 | 60.536 | 113.815 | 1.00 | 71.90 | C |
| ATOM | 10831 | CZ | PHE | C | 118 | 50.239 | 59.516 | 114.666 | 1.00 | 72.99 | C |
| ATOM | 10833 | CE2 | PHE | C | 118 | 51.383 | 59.572 | 115.442 | 1.00 | 74.93 | C |
| ATOM | 10835 | CD2 | PHE | C | 118 | 52.232 | 60.651 | 115.391 | 1.00 | 74.47 | C |
| ATOM | 10837 | C | PHE | C | 118 | 53.975 | 64.915 | 115.393 | 1.00 | 78.41 | C |
| ATOM | 10838 | O | PHE | C | 118 | 54.268 | 65.444 | 114.286 | 1.00 | 76.97 | O |
| ATOM | 10839 | N | PRO | C | 119 | 54.675 | 65.181 | 116.500 | 1.00 | 82.21 | N |
| ATOM | 10840 | CA | PRO | C | 119 | 55.920 | 65.957 | 116.393 | 1.00 | 84.25 | C |
| ATOM | 10842 | CB | PRO | C | 119 | 56.542 | 65.838 | 117.779 | 1.00 | 88.82 | C |
| ATOM | 10845 | CG | PRO | C | 119 | 55.382 | 65.517 | 118.696 | 1.00 | 89.91 | C |
| ATOM | 10848 | CD | PRO | C | 119 | 54.374 | 64.797 | 117.892 | 1.00 | 85.44 | C |
| ATOM | 10851 | C | PRO | C | 119 | 56.828 | 65.320 | 115.316 | 1.00 | 82.79 | C |
| ATOM | 10852 | O | PRO | C | 119 | 56.648 | 64.125 | 114.994 | 1.00 | 82.06 | O |
| ATOM | 10853 | N | PRO | C | 120 | 57.709 | 66.107 | 114.711 | 1.00 | 82.76 | N |
| ATOM | 10854 | CA | PRO | C | 120 | 58.752 | 65.563 | 113.838 | 1.00 | 82.28 | C |
| ATOM | 10856 | CB | PRO | C | 120 | 59.444 | 66.815 | 113.282 | 1.00 | 82.97 | C |
| ATOM | 10859 | CG | PRO | C | 120 | 59.057 | 67.942 | 114.183 | 1.00 | 85.25 | C |
| ATOM | 10862 | CD | PRO | C | 120 | 57.728 | 67.580 | 114.747 | 1.00 | 84.01 | C |
| ATOM | 10865 | C | PRO | C | 120 | 59.749 | 64.678 | 114.568 | 1.00 | 85.50 | C |
| ATOM | 10866 | O | PRO | C | 120 | 60.140 | 64.963 | 115.715 | 1.00 | 90.03 | O |
| ATOM | 10867 | N | SER | C | 121 | 60.184 | 63.637 | 113.886 | 1.00 | 84.53 | N |
| ATOM | 10869 | CA | SER | C | 121 | 61.025 | 62.639 | 114.484 | 1.00 | 88.74 | C |
| ATOM | 10871 | CB | SER | C | 121 | 60.927 | 61.392 | 113.675 | 1.00 | 87.48 | C |
| ATOM | 10874 | OG | SER | C | 121 | 61.718 | 61.641 | 112.529 | 1.00 | 88.53 | O |
| ATOM | 10876 | C | SER | C | 121 | 62.513 | 63.037 | 114.538 | 1.00 | 92.58 | C |
| ATOM | 10877 | O | SER | C | 121 | 62.974 | 63.943 | 113.830 | 1.00 | 92.02 | O |
| ATOM | 10878 | N | ASP | C | 122 | 63.254 | 62.310 | 115.374 | 1.00 | 96.87 | N |
| ATOM | 10880 | CA | ASP | C | 122 | 64.668 | 62.564 | 115.636 | 1.00 | 100.88 | C |
| ATOM | 10882 | CB | ASP | C | 122 | 65.212 | 61.403 | 116.516 | 1.00 | 106.35 | C |
| ATOM | 10885 | CG | ASP | C | 122 | 64.930 | 61.583 | 118.022 | 1.00 | 110.21 | C |
| ATOM | 10886 | OD1 | ASP | C | 122 | 63.991 | 62.303 | 118.397 | 1.00 | 109.83 | O |
| ATOM | 10887 | OD2 | ASP | C | 122 | 65.610 | 61.032 | 118.913 | 1.00 | 115.97 | O |
| ATOM | 10888 | C | ASP | C | 122 | 65.523 | 62.705 | 114.353 | 1.00 | 99.41 | C |
| ATOM | 10889 | O | ASP | C | 122 | 65.919 | 63.808 | 113.992 | 1.00 | 97.33 | O |
| ATOM | 10890 | N | GLU | C | 123 | 65.761 | 61.552 | 113.698 | 1.00 | 99.99 | N |
| ATOM | 10892 | CA | GLU | C | 123 | 66.475 | 61.402 | 112.414 | 1.00 | 99.67 | C |
| ATOM | 10894 | CB | GLU | C | 123 | 66.175 | 60.022 | 111.737 | 1.00 | 98.14 | C |
| ATOM | 10901 | C | GLU | C | 123 | 66.137 | 62.569 | 111.477 | 1.00 | 96.68 | C |
| ATOM | 10902 | O | GLU | C | 123 | 67.038 | 63.239 | 110.987 | 1.00 | 98.14 | O |
| ATOM | 10903 | N | GLN | C | 124 | 64.841 | 62.842 | 111.273 | 1.00 | 93.61 | N |
| ATOM | 10905 | CA | GLN | C | 124 | 64.398 | 63.880 | 110.305 | 1.00 | 90.64 | C |
| ATOM | 10907 | CB | GLN | C | 124 | 62.846 | 63.927 | 110.138 | 1.00 | 86.51 | C |
| ATOM | 10910 | CG | GLN | C | 124 | 62.362 | 64.800 | 108.958 | 1.00 | 82.22 | C |
| ATOM | 10913 | CD | GLN | C | 124 | 60.869 | 65.118 | 108.916 | 1.00 | 78.52 | C |
| ATOM | 10914 | OE1 | GLN | C | 124 | 60.108 | 64.980 | 109.888 | 1.00 | 75.18 | O |
| ATOM | 10915 | NE2 | GLN | C | 124 | 60.451 | 65.558 | 107.759 | 1.00 | 77.75 | N |
| ATOM | 10918 | C | GLN | C | 124 | 64.956 | 65.278 | 110.658 | 1.00 | 92.63 | C |
| ATOM | 10919 | O | GLN | C | 124 | 65.541 | 65.966 | 109.784 | 1.00 | 92.86 | O |
| ATOM | 10920 | N | LEU | C | 125 | 64.788 | 65.691 | 111.917 | 1.00 | 94.07 | N |
| ATOM | 10922 | CA | LEU | C | 125 | 65.326 | 66.981 | 112.331 | 1.00 | 96.57 | C |
| ATOM | 10924 | CB | LEU | C | 125 | 64.896 | 67.338 | 113.755 | 1.00 | 98.49 | C |
| ATOM | 10927 | CG | LEU | C | 125 | 63.933 | 68.526 | 113.913 | 1.00 | 97.09 | C |
| ATOM | 10929 | CD1 | LEU | C | 125 | 62.846 | 68.157 | 114.932 | 1.00 | 96.51 | C |
| ATOM | 10933 | CD2 | LEU | C | 125 | 64.671 | 69.836 | 114.312 | 1.00 | 99.62 | C |
| ATOM | 10937 | C | LEU | C | 125 | 66.857 | 66.986 | 112.192 | 1.00 | 100.24 | C |
| ATOM | 10938 | O | LEU | C | 125 | 67.463 | 68.054 | 112.104 | 1.00 | 102.53 | O |
| ATOM | 10939 | N | LYS | C | 126 | 67.467 | 65.794 | 112.178 | 1.00 | 101.49 | N |
| ATOM | 10941 | CA | LYS | C | 126 | 68.902 | 65.655 | 111.893 | 1.00 | 105.04 | C |
| ATOM | 10943 | CB | LYS | C | 126 | 69.455 | 64.271 | 112.318 | 1.00 | 107.49 | C |
| ATOM | 10950 | C | LYS | C | 126 | 69.171 | 65.943 | 110.397 | 1.00 | 103.41 | C |
| ATOM | 10951 | O | LYS | C | 126 | 70.085 | 66.726 | 110.076 | 1.00 | 106.04 | O |
| ATOM | 10952 | N | SER | C | 127 | 68.371 | 65.337 | 109.510 | 1.00 | 99.35 | N |
| ATOM | 10954 | CA | SER | C | 127 | 68.423 | 65.607 | 108.082 | 1.00 | 97.89 | C |
| ATOM | 10956 | CB | SER | C | 127 | 67.356 | 64.775 | 107.347 | 1.00 | 95.15 | C |
| ATOM | 10959 | OG | SER | C | 127 | 66.142 | 65.501 | 107.072 | 1.00 | 92.42 | O |
| ATOM | 10961 | C | SER | C | 127 | 68.227 | 67.087 | 107.758 | 1.00 | 97.29 | C |
| ATOM | 10962 | O | SER | C | 127 | 68.571 | 67.510 | 106.671 | 1.00 | 97.87 | O |
| ATOM | 10963 | N | GLY | C | 128 | 67.655 | 67.860 | 108.685 | 1.00 | 96.84 | N |
| ATOM | 10965 | CA | GLY | C | 128 | 67.451 | 69.292 | 108.505 | 1.00 | 97.37 | C |
| ATOM | 10968 | C | GLY | C | 128 | 66.058 | 69.738 | 108.039 | 1.00 | 94.13 | C |
| ATOM | 10969 | O | GLY | C | 128 | 65.832 | 70.917 | 107.753 | 1.00 | 94.42 | O |
| ATOM | 10970 | N | THR | C | 129 | 65.093 | 68.828 | 107.976 | 1.00 | 91.09 | N |
| ATOM | 10972 | CA | THR | C | 129 | 63.711 | 69.278 | 107.773 | 1.00 | 88.29 | C |
| ATOM | 10974 | CB | THR | C | 129 | 63.245 | 68.917 | 106.341 | 1.00 | 86.24 | C |
| ATOM | 10976 | OG1 | THR | C | 129 | 64.353 | 69.086 | 105.439 | 1.00 | 88.83 | O |
| ATOM | 10978 | CG2 | THR | C | 129 | 62.258 | 69.966 | 105.811 | 1.00 | 85.12 | C |
| ATOM | 10982 | C | THR | C | 129 | 62.732 | 68.840 | 108.882 | 1.00 | 86.18 | C |
| ATOM | 10983 | O | THR | C | 129 | 63.036 | 67.960 | 109.684 | 1.00 | 87.16 | O |
| ATOM | 10984 | N | ALA | C | 130 | 61.593 | 69.530 | 108.955 | 1.00 | 84.15 | N |
| ATOM | 10986 | CA | ALA | C | 130 | 60.525 | 69.238 | 109.908 | 1.00 | 81.98 | C |
| ATOM | 10988 | CB | ALA | C | 130 | 60.318 | 70.399 | 110.827 | 1.00 | 84.47 | C |
| ATOM | 10992 | C | ALA | C | 130 | 59.244 | 68.958 | 109.157 | 1.00 | 78.03 | C |
| ATOM | 10993 | O | ALA | C | 130 | 58.773 | 69.810 | 108.392 | 1.00 | 76.36 | O |
| ATOM | 10994 | N | SER | C | 131 | 58.708 | 67.749 | 109.369 | 1.00 | 76.12 | N |
| ATOM | 10996 | CA | SER | C | 131 | 57.410 | 67.320 | 108.816 | 1.00 | 72.65 | C |
| ATOM | 10998 | CB | SER | C | 131 | 57.553 | 66.111 | 107.877 | 1.00 | 70.72 | C |
| ATOM | 11001 | OG | SER | C | 131 | 57.712 | 66.485 | 106.501 | 1.00 | 70.02 | O |
| ATOM | 11003 | C | SER | C | 131 | 56.496 | 66.992 | 110.011 | 1.00 | 72.77 | C |
| ATOM | 11004 | O | SER | C | 131 | 56.814 | 66.125 | 110.855 | 1.00 | 74.28 | O |
| ATOM | 11005 | N | VAL | C | 132 | 55.378 | 67.710 | 110.105 | 1.00 | 71.88 | N |
| ATOM | 11007 | CA | VAL | C | 132 | 54.471 | 67.565 | 111.239 | 1.00 | 72.03 | C |
| ATOM | 11009 | CB | VAL | C | 132 | 54.174 | 68.881 | 111.954 | 1.00 | 73.84 | C |
| ATOM | 11011 | CG1 | VAL | C | 132 | 53.490 | 68.605 | 113.281 | 1.00 | 75.05 | C |
| ATOM | 11015 | CG2 | VAL | C | 132 | 55.443 | 69.693 | 112.128 | 1.00 | 77.41 | C |
| ATOM | 11019 | C | VAL | C | 132 | 53.191 | 67.089 | 110.658 | 1.00 | 69.08 | C |
| ATOM | 11020 | O | VAL | C | 132 | 52.743 | 67.657 | 109.685 | 1.00 | 67.17 | O |
| ATOM | 11021 | N | VAL | C | 133 | 52.591 | 66.083 | 111.287 | 1.00 | 68.84 | N |
| ATOM | 11023 | CA | VAL | C | 133 | 51.465 | 65.357 | 110.700 | 1.00 | 66.13 | C |
| ATOM | 11025 | CB | VAL | C | 133 | 51.861 | 63.890 | 110.444 | 1.00 | 66.16 | C |
| ATOM | 11027 | CG1 | VAL | C | 133 | 50.771 | 63.077 | 109.757 | 1.00 | 62.31 | C |
| ATOM | 11031 | CG2 | VAL | C | 133 | 53.162 | 63.852 | 109.672 | 1.00 | 68.50 | C |
| ATOM | 11035 | C | VAL | C | 133 | 50.262 | 65.317 | 111.596 | 1.00 | 66.14 | C |
| ATOM | 11036 | O | VAL | C | 133 | 50.346 | 65.030 | 112.772 | 1.00 | 68.15 | O |
| ATOM | 11037 | N | CYS | C | 134 | 49.125 | 65.563 | 110.990 | 1.00 | 64.89 | N |
| ATOM | 11039 | CA | CYS | C | 134 | 47.844 | 65.261 | 111.575 | 1.00 | 64.95 | C |
| ATOM | 11041 | CB | CYS | C | 134 | 46.938 | 66.457 | 111.408 | 1.00 | 65.26 | C |
| ATOM | 11044 | SG | CYS | C | 134 | 46.037 | 66.697 | 112.941 | 1.00 | 72.19 | S |
| ATOM | 11045 | C | CYS | C | 134 | 47.197 | 64.094 | 110.858 | 1.00 | 62.06 | C |
| ATOM | 11046 | O | CYS | C | 134 | 47.449 | 63.862 | 109.674 | 1.00 | 61.20 | O |
| ATOM | 11047 | N | LEU | C | 135 | 46.337 | 63.380 | 111.568 | 1.00 | 61.40 | N |
| ATOM | 11049 | CA | LEU | C | 135 | 45.617 | 62.235 | 111.027 | 1.00 | 58.71 | C |
| ATOM | 11051 | CB | LEU | C | 135 | 46.183 | 60.986 | 111.655 | 1.00 | 60.46 | C |
| ATOM | 11054 | CG | LEU | C | 135 | 45.430 | 59.670 | 111.520 | 1.00 | 61.31 | C |
| ATOM | 11056 | CD1 | LEU | C | 135 | 45.114 | 59.308 | 110.103 | 1.00 | 60.36 | C |
| ATOM | 11060 | CD2 | LEU | C | 135 | 46.276 | 58.559 | 112.078 | 1.00 | 63.76 | C |
| ATOM | 11064 | C | LEU | C | 135 | 44.221 | 62.435 | 111.468 | 1.00 | 57.46 | C |
| ATOM | 11065 | O | LEU | C | 135 | 43.984 | 62.674 | 112.610 | 1.00 | 58.68 | O |
| ATOM | 11066 | N | LEU | C | 136 | 43.282 | 62.449 | 110.566 | 1.00 | 56.07 | N |
| ATOM | 11068 | CA | LEU | C | 136 | 41.871 | 62.381 | 110.951 | 1.00 | 56.20 | C |
| ATOM | 11070 | CB | LEU | C | 136 | 41.019 | 63.385 | 110.192 | 1.00 | 54.74 | C |
| ATOM | 11073 | CG | LEU | C | 136 | 41.477 | 64.822 | 109.904 | 1.00 | 55.75 | C |
| ATOM | 11075 | CD1 | LEU | C | 136 | 40.252 | 65.754 | 109.846 | 1.00 | 56.08 | C |
| ATOM | 11079 | CD2 | LEU | C | 136 | 42.517 | 65.363 | 110.864 | 1.00 | 57.95 | C |
| ATOM | 11083 | C | LEU | C | 136 | 41.455 | 60.970 | 110.567 | 1.00 | 56.76 | C |
| ATOM | 11084 | O | LEU | C | 136 | 41.558 | 60.579 | 109.377 | 1.00 | 54.98 | O |
| ATOM | 11085 | N | ASN | C | 137 | 41.010 | 60.175 | 111.543 | 1.00 | 59.04 | N |
| ATOM | 11087 | CA | ASN | C | 137 | 40.813 | 58.746 | 111.242 | 1.00 | 60.23 | C |
| ATOM | 11089 | CB | ASN | C | 137 | 41.551 | 57.792 | 112.223 | 1.00 | 63.94 | C |
| ATOM | 11092 | CG | ASN | C | 137 | 41.899 | 56.418 | 111.560 | 1.00 | 66.89 | C |
| ATOM | 11093 | OD1 | ASN | C | 137 | 41.856 | 56.288 | 110.338 | 1.00 | 70.32 | O |
| ATOM | 11094 | ND2 | ASN | C | 137 | 42.199 | 55.407 | 112.361 | 1.00 | 71.93 | N |
| ATOM | 11097 | C | ASN | C | 137 | 39.365 | 58.402 | 111.194 | 1.00 | 58.92 | C |
| ATOM | 11098 | O | ASN | C | 137 | 38.607 | 58.929 | 111.965 | 1.00 | 59.90 | O |
| ATOM | 11099 | N | ASN | C | 138 | 39.015 | 57.519 | 110.275 | 1.00 | 58.00 | N |
| ATOM | 11101 | CA | ASN | C | 138 | 37.670 | 56.976 | 110.095 | 1.00 | 57.80 | C |
| ATOM | 11103 | CB | ASN | C | 138 | 37.502 | 55.768 | 110.969 | 1.00 | 60.82 | C |
| ATOM | 11106 | CG | ASN | C | 138 | 38.594 | 54.763 | 110.761 | 1.00 | 63.86 | C |
| ATOM | 11107 | OD1 | ASN | C | 138 | 39.038 | 54.501 | 109.625 | 1.00 | 64.97 | O |
| ATOM | 11108 | ND2 | ASN | C | 138 | 39.057 | 54.201 | 111.853 | 1.00 | 67.79 | N |
| ATOM | 11111 | C | ASN | C | 138 | 36.531 | 57.965 | 110.296 | 1.00 | 56.57 | C |
| ATOM | 11112 | O | ASN | C | 138 | 35.732 | 57.824 | 111.235 | 1.00 | 57.27 | O |
| ATOM | 11113 | N | PHE | C | 139 | 36.479 | 58.985 | 109.420 | 1.00 | 54.48 | N |
| ATOM | 11115 | CA | PHE | C | 139 | 35.331 | 59.891 | 109.404 | 1.00 | 53.81 | C |
| ATOM | 11117 | CB | PHE | C | 139 | 35.790 | 61.326 | 109.616 | 1.00 | 53.42 | C |
| ATOM | 11120 | CG | PHE | C | 139 | 36.608 | 61.856 | 108.508 | 1.00 | 51.95 | C |
| ATOM | 11121 | CD1 | PHE | C | 139 | 36.007 | 62.464 | 107.425 | 1.00 | 50.55 | C |
| ATOM | 11123 | CE1 | PHE | C | 139 | 36.768 | 62.941 | 106.395 | 1.00 | 50.36 | C |
| ATOM | 11125 | CZ | PHE | C | 139 | 38.144 | 62.850 | 106.440 | 1.00 | 48.23 | C |
| ATOM | 11127 | CE2 | PHE | C | 139 | 38.739 | 62.256 | 107.503 | 1.00 | 49.74 | C |
| ATOM | 11129 | CD2 | PHE | C | 139 | 37.976 | 61.749 | 108.536 | 1.00 | 50.89 | C |
| ATOM | 11131 | C | PHE | C | 139 | 34.416 | 59.794 | 108.162 | 1.00 | 52.65 | C |
| ATOM | 11132 | O | PHE | C | 139 | 34.751 | 59.186 | 107.164 | 1.00 | 50.71 | O |
| ATOM | 11133 | N | TYR | C | 140 | 33.241 | 60.391 | 108.281 | 1.00 | 52.90 | N |
| ATOM | 11135 | CA | TYR | C | 140 | 32.274 | 60.479 | 107.190 | 1.00 | 53.13 | C |
| ATOM | 11137 | CB | TYR | C | 140 | 31.467 | 59.199 | 107.034 | 1.00 | 54.47 | C |
| ATOM | 11140 | CG | TYR | C | 140 | 30.597 | 59.241 | 105.814 | 1.00 | 55.53 | C |
| ATOM | 11141 | CD1 | TYR | C | 140 | 31.066 | 58.769 | 104.599 | 1.00 | 56.84 | C |
| ATOM | 11143 | CE1 | TYR | C | 140 | 30.306 | 58.850 | 103.463 | 1.00 | 56.96 | C |
| ATOM | 11145 | CZ | TYR | C | 140 | 29.054 | 59.386 | 103.533 | 1.00 | 58.04 | C |
| ATOM | 11146 | OH | TYR | C | 140 | 28.331 | 59.472 | 102.372 | 1.00 | 60.87 | O |
| ATOM | 11148 | CE2 | TYR | C | 140 | 28.565 | 59.856 | 104.720 | 1.00 | 57.39 | C |
| ATOM | 11150 | CD2 | TYR | C | 140 | 29.344 | 59.792 | 105.851 | 1.00 | 56.47 | C |
| ATOM | 11152 | C | TYR | C | 140 | 31.327 | 61.564 | 107.646 | 1.00 | 53.37 | C |
| ATOM | 11153 | O | TYR | C | 140 | 30.973 | 61.600 | 108.799 | 1.00 | 54.87 | O |
| ATOM | 11154 | N | PRO | C | 141 | 30.932 | 62.477 | 106.799 | 1.00 | 52.72 | N |
| ATOM | 11155 | CA | PRO | C | 141 | 31.297 | 62.520 | 105.408 | 1.00 | 52.43 | C |
| ATOM | 11157 | CB | PRO | C | 141 | 30.176 | 63.388 | 104.835 | 1.00 | 53.29 | C |
| ATOM | 11160 | CG | PRO | C | 141 | 29.996 | 64.367 | 105.828 | 1.00 | 53.92 | C |
| ATOM | 11163 | CD | PRO | C | 141 | 30.119 | 63.638 | 107.168 | 1.00 | 53.63 | C |
| ATOM | 11166 | C | PRO | C | 141 | 32.700 | 63.141 | 105.142 | 1.00 | 51.46 | C |
| ATOM | 11167 | O | PRO | C | 141 | 33.371 | 63.582 | 106.018 | 1.00 | 50.16 | O |
| ATOM | 11168 | N | ARG | C | 142 | 33.106 | 63.171 | 103.880 | 1.00 | 52.37 | N |
| ATOM | 11170 | CA | ARG | C | 142 | 34.467 | 63.464 | 103.538 | 1.00 | 52.21 | C |
| ATOM | 11172 | CB | ARG | C | 142 | 34.734 | 63.188 | 102.077 | 1.00 | 53.40 | C |
| ATOM | 11175 | CG | ARG | C | 142 | 36.078 | 63.698 | 101.630 | 1.00 | 56.32 | C |
| ATOM | 11178 | CD | ARG | C | 142 | 36.554 | 63.071 | 100.360 | 1.00 | 60.42 | C |
| ATOM | 11181 | NE | ARG | C | 142 | 37.713 | 63.814 | 99.826 | 1.00 | 62.68 | N |
| ATOM | 11183 | CZ | ARG | C | 142 | 38.378 | 63.446 | 98.735 | 1.00 | 62.72 | C |
| ATOM | 11184 | NH1 | ARG | C | 142 | 37.965 | 62.338 | 98.098 | 1.00 | 67.02 | N |
| ATOM | 11187 | NH2 | ARG | C | 142 | 39.433 | 64.150 | 98.286 | 1.00 | 59.04 | N |
| ATOM | 11190 | C | ARG | C | 142 | 34.803 | 64.879 | 103.799 | 1.00 | 52.89 | C |
| ATOM | 11191 | O | ARG | C | 142 | 35.994 | 65.178 | 104.043 | 1.00 | 52.87 | O |
| ATOM | 11192 | N | GLU | C | 143 | 33.813 | 65.776 | 103.712 | 1.00 | 54.26 | N |
| ATOM | 11194 | CA | GLU | C | 143 | 34.158 | 67.175 | 103.987 | 1.00 | 56.18 | C |
| ATOM | 11196 | CB | GLU | C | 143 | 33.053 | 68.169 | 103.642 | 1.00 | 58.86 | C |
| ATOM | 11199 | CG | GLU | C | 143 | 33.596 | 69.598 | 103.729 | 1.00 | 63.26 | C |
| ATOM | 11202 | CD | GLU | C | 143 | 32.567 | 70.696 | 103.455 | 1.00 | 70.77 | C |
| ATOM | 11203 | OE1 | GLU | C | 143 | 31.399 | 70.575 | 103.939 | 1.00 | 72.09 | O |
| ATOM | 11204 | OE2 | GLU | C | 143 | 32.940 | 71.714 | 102.782 | 1.00 | 74.74 | O |
| ATOM | 11205 | C | GLU | C | 143 | 34.663 | 67.386 | 105.427 | 1.00 | 55.52 | C |
| ATOM | 11206 | O | GLU | C | 143 | 33.912 | 67.386 | 106.409 | 1.00 | 55.37 | O |
| ATOM | 11207 | N | ALA | C | 144 | 35.975 | 67.507 | 105.516 | 1.00 | 55.94 | N |
| ATOM | 11209 | CA | ALA | C | 144 | 36.665 | 67.970 | 106.712 | 1.00 | 57.00 | C |
| ATOM | 11211 | CB | ALA | C | 144 | 37.587 | 66.852 | 107.213 | 1.00 | 55.95 | C |
| ATOM | 11215 | C | ALA | C | 144 | 37.478 | 69.253 | 106.372 | 1.00 | 58.66 | C |
| ATOM | 11216 | O | ALA | C | 144 | 37.631 | 69.622 | 105.214 | 1.00 | 59.65 | O |
| ATOM | 11217 | N | LYS | C | 145 | 38.038 | 69.884 | 107.380 | 1.00 | 59.98 | N |
| ATOM | 11219 | CA | LYS | C | 145 | 38.880 | 71.055 | 107.194 | 1.00 | 62.18 | C |
| ATOM | 11221 | CB | LYS | C | 145 | 37.975 | 72.309 | 107.181 | 1.00 | 65.67 | C |
| ATOM | 11224 | CG | LYS | C | 145 | 38.627 | 73.759 | 107.072 | 1.00 | 70.00 | C |
| ATOM | 11227 | CD | LYS | C | 145 | 37.728 | 74.817 | 107.895 | 1.00 | 74.41 | C |
| ATOM | 11230 | CE | LYS | C | 145 | 37.394 | 76.202 | 107.210 | 1.00 | 78.41 | C |
| ATOM | 11233 | NZ | LYS | C | 145 | 37.443 | 76.330 | 105.690 | 1.00 | 76.16 | N |
| ATOM | 11237 | C | LYS | C | 145 | 39.948 | 71.077 | 108.306 | 1.00 | 62.49 | C |
| ATOM | 11238 | O | LYS | C | 145 | 39.714 | 70.784 | 109.493 | 1.00 | 62.13 | O |
| ATOM | 11239 | N | VAL | C | 146 | 41.152 | 71.415 | 107.909 | 1.00 | 63.00 | N |
| ATOM | 11241 | CA | VAL | C | 146 | 42.268 | 71.334 | 108.824 | 1.00 | 63.55 | C |
| ATOM | 11243 | CB | VAL | C | 146 | 43.183 | 70.193 | 108.431 | 1.00 | 61.33 | C |
| ATOM | 11245 | CG1 | VAL | C | 146 | 44.510 | 70.267 | 109.248 | 1.00 | 63.03 | C |
| ATOM | 11249 | CG2 | VAL | C | 146 | 42.446 | 68.892 | 108.609 | 1.00 | 58.25 | C |
| ATOM | 11253 | C | VAL | C | 146 | 43.060 | 72.595 | 108.730 | 1.00 | 66.29 | C |
| ATOM | 11254 | O | VAL | C | 146 | 43.628 | 72.875 | 107.681 | 1.00 | 66.84 | O |
| ATOM | 11255 | N | GLN | C | 147 | 43.095 | 73.350 | 109.817 | 1.00 | 68.87 | N |
| ATOM | 11257 | CA | GLN | C | 147 | 43.905 | 74.536 | 109.882 | 1.00 | 72.37 | C |
| ATOM | 11259 | CB | GLN | C | 147 | 43.099 | 75.734 | 110.396 | 1.00 | 76.83 | C |
| ATOM | 11262 | CG | GLN | C | 147 | 42.317 | 76.514 | 109.337 | 1.00 | 77.62 | C |
| ATOM | 11265 | CD | GLN | C | 147 | 41.165 | 77.347 | 109.930 | 1.00 | 80.53 | C |
| ATOM | 11266 | OE1 | GLN | C | 147 | 41.367 | 78.499 | 110.283 | 1.00 | 88.62 | O |
| ATOM | 11267 | NE2 | GLN | C | 147 | 39.980 | 76.775 | 110.015 | 1.00 | 74.96 | N |
| ATOM | 11270 | C | GLN | C | 147 | 45.058 | 74.207 | 110.804 | 1.00 | 72.82 | C |
| ATOM | 11271 | O | GLN | C | 147 | 44.920 | 73.510 | 111.778 | 1.00 | 71.65 | O |
| ATOM | 11272 | N | TRP | C | 148 | 46.224 | 74.684 | 110.443 | 1.00 | 74.99 | N |
| ATOM | 11274 | CA | TRP | C | 148 | 47.392 | 74.550 | 111.287 | 1.00 | 76.89 | C |
| ATOM | 11276 | CB | TRP | C | 148 | 48.588 | 74.068 | 110.465 | 1.00 | 74.83 | C |
| ATOM | 11279 | CG | TRP | C | 148 | 48.711 | 72.619 | 110.138 | 1.00 | 70.59 | C |
| ATOM | 11280 | CD1 | TRP | C | 148 | 48.353 | 72.042 | 108.978 | 1.00 | 67.59 | C |
| ATOM | 11282 | NE1 | TRP | C | 148 | 48.676 | 70.711 | 108.984 | 1.00 | 63.05 | N |
| ATOM | 11284 | CE2 | TRP | C | 148 | 49.305 | 70.418 | 110.155 | 1.00 | 65.79 | C |
| ATOM | 11285 | CD2 | TRP | C | 148 | 49.343 | 71.584 | 110.905 | 1.00 | 69.46 | C |
| ATOM | 11286 | CE3 | TRP | C | 148 | 49.938 | 71.526 | 112.141 | 1.00 | 72.82 | C |
| ATOM | 11288 | CZ3 | TRP | C | 148 | 50.457 | 70.314 | 112.584 | 1.00 | 71.92 | C |
| ATOM | 11290 | CH2 | TRP | C | 148 | 50.410 | 69.202 | 111.822 | 1.00 | 68.74 | C |
| ATOM | 11292 | CZ2 | TRP | C | 148 | 49.838 | 69.223 | 110.603 | 1.00 | 67.06 | C |
| ATOM | 11294 | C | TRP | C | 148 | 47.705 | 75.955 | 111.937 | 1.00 | 82.80 | C |
| ATOM | 11295 | O | TRP | C | 148 | 47.740 | 77.001 | 111.243 | 1.00 | 84.38 | O |
| ATOM | 11296 | N | LYS | C | 149 | 47.929 | 75.943 | 113.262 | 1.00 | 85.63 | N |
| ATOM | 11298 | CA | LYS | C | 149 | 48.203 | 77.147 | 114.050 | 1.00 | 91.54 | C |
| ATOM | 11300 | CB | LYS | C | 149 | 47.065 | 77.424 | 115.063 | 1.00 | 94.11 | C |
| ATOM | 11303 | CG | LYS | C | 149 | 45.657 | 77.643 | 114.457 | 1.00 | 92.57 | C |
| ATOM | 11306 | CD | LYS | C | 149 | 44.608 | 77.959 | 115.522 | 1.00 | 95.47 | C |
| ATOM | 11309 | CE | LYS | C | 149 | 43.170 | 77.612 | 115.088 | 1.00 | 93.07 | C |
| ATOM | 11312 | NZ | LYS | C | 149 | 42.369 | 76.679 | 116.029 | 1.00 | 90.06 | N |
| ATOM | 11316 | C | LYS | C | 149 | 49.548 | 76.978 | 114.773 | 1.00 | 93.58 | C |
| ATOM | 11317 | O | LYS | C | 149 | 49.876 | 75.896 | 115.279 | 1.00 | 91.51 | O |
| ATOM | 11318 | N | VAL | C | 150 | 50.315 | 78.062 | 114.807 | 1.00 | 97.73 | N |
| ATOM | 11320 | CA | VAL | C | 150 | 51.624 | 78.061 | 115.384 | 1.00 | 100.07 | C |
| ATOM | 11322 | CB | VAL | C | 150 | 52.631 | 78.262 | 114.275 | 1.00 | 99.22 | C |
| ATOM | 11324 | CG1 | VAL | C | 150 | 54.029 | 78.444 | 114.828 | 1.00 | 104.37 | C |
| ATOM | 11328 | CG2 | VAL | C | 150 | 52.601 | 77.096 | 113.333 | 1.00 | 93.65 | C |
| ATOM | 11332 | C | VAL | C | 150 | 51.629 | 79.233 | 116.329 | 1.00 | 107.01 | C |
| ATOM | 11333 | O | VAL | C | 150 | 51.874 | 80.369 | 115.916 | 1.00 | 111.28 | O |
| ATOM | 11334 | N | ASP | C | 151 | 51.334 | 78.985 | 117.600 | 1.00 | 109.29 | N |
| ATOM | 11336 | CA | ASP | C | 151 | 51.128 | 80.083 | 118.573 | 1.00 | 116.46 | C |
| ATOM | 11338 | CB | ASP | C | 151 | 52.361 | 81.004 | 118.708 | 1.00 | 121.70 | C |
| ATOM | 11341 | CG | ASP | C | 151 | 53.612 | 80.265 | 119.111 | 1.00 | 119.58 | C |
| ATOM | 11342 | OD1 | ASP | C | 151 | 53.619 | 79.651 | 120.194 | 1.00 | 118.15 | O |
| ATOM | 11343 | OD2 | ASP | C | 151 | 54.640 | 80.248 | 118.410 | 1.00 | 116.55 | O |
| ATOM | 11344 | C | ASP | C | 151 | 49.927 | 80.929 | 118.156 | 1.00 | 118.10 | C |
| ATOM | 11345 | O | ASP | C | 151 | 49.762 | 82.048 | 118.615 | 1.00 | 124.53 | O |
| ATOM | 11346 | N | ASN | C | 152 | 49.106 | 80.368 | 117.273 | 1.00 | 112.89 | N |
| ATOM | 11348 | CA | ASN | C | 152 | 47.884 | 80.991 | 116.737 | 1.00 | 113.81 | C |
| ATOM | 11350 | CB | ASN | C | 152 | 46.964 | 81.550 | 117.853 | 1.00 | 119.38 | C |
| ATOM | 11353 | CG | ASN | C | 152 | 46.467 | 80.472 | 118.794 | 1.00 | 117.08 | C |
| ATOM | 11354 | OD1 | ASN | C | 152 | 45.498 | 79.741 | 118.489 | 1.00 | 111.65 | O |
| ATOM | 11355 | ND2 | ASN | C | 152 | 47.133 | 80.356 | 119.948 | 1.00 | 119.53 | N |
| ATOM | 11358 | C | ASN | C | 152 | 48.039 | 82.015 | 115.598 | 1.00 | 115.75 | C |
| ATOM | 11359 | O | ASN | C | 152 | 47.197 | 82.882 | 115.467 | 1.00 | 119.93 | O |
| ATOM | 11360 | N | ALA | C | 153 | 49.086 | 81.916 | 114.773 | 1.00 | 113.90 | N |
| ATOM | 11362 | CA | ALA | C | 153 | 49.016 | 82.419 | 113.383 | 1.00 | 112.97 | C |
| ATOM | 11364 | CB | ALA | C | 153 | 50.362 | 83.049 | 112.914 | 1.00 | 115.78 | C |
| ATOM | 11368 | C | ALA | C | 153 | 48.582 | 81.215 | 112.489 | 1.00 | 105.40 | C |
| ATOM | 11369 | O | ALA | C | 153 | 49.190 | 80.118 | 112.536 | 1.00 | 100.44 | O |
| ATOM | 11370 | N | LEU | C | 154 | 47.490 | 81.404 | 111.738 | 1.00 | 104.22 | N |
| ATOM | 11372 | CA | LEU | C | 154 | 47.062 | 80.433 | 110.725 | 1.00 | 98.09 | C |
| ATOM | 11374 | CB | LEU | C | 154 | 45.803 | 80.913 | 109.980 | 1.00 | 99.06 | C |
| ATOM | 11377 | CG | LEU | C | 154 | 44.449 | 80.931 | 110.712 | 1.00 | 100.59 | C |
| ATOM | 11379 | CD1 | LEU | C | 154 | 44.035 | 82.369 | 111.263 | 1.00 | 107.76 | C |
| ATOM | 11383 | CD2 | LEU | C | 154 | 43.376 | 80.317 | 109.788 | 1.00 | 96.43 | C |
| ATOM | 11387 | C | LEU | C | 154 | 48.178 | 80.250 | 109.706 | 1.00 | 96.18 | C |
| ATOM | 11388 | O | LEU | C | 154 | 48.764 | 81.229 | 109.238 | 1.00 | 99.72 | O |
| ATOM | 11389 | N | GLN | C | 155 | 48.466 | 78.997 | 109.357 | 1.00 | 90.49 | N |
| ATOM | 11391 | CA | GLN | C | 155 | 49.477 | 78.703 | 108.346 | 1.00 | 88.65 | C |
| ATOM | 11393 | CB | GLN | C | 155 | 50.264 | 77.418 | 108.660 | 1.00 | 84.72 | C |
| ATOM | 11396 | CG | GLN | C | 155 | 51.045 | 77.407 | 109.970 | 1.00 | 85.90 | C |
| ATOM | 11399 | CD | GLN | C | 155 | 51.946 | 78.589 | 110.099 | 1.00 | 91.43 | C |
| ATOM | 11400 | OE1 | GLN | C | 155 | 52.738 | 78.847 | 109.204 | 1.00 | 93.14 | O |
| ATOM | 11401 | NE2 | GLN | C | 155 | 51.820 | 79.336 | 111.198 | 1.00 | 95.40 | N |
| ATOM | 11404 | C | GLN | C | 155 | 48.752 | 78.564 | 107.025 | 1.00 | 86.54 | C |
| ATOM | 11405 | O | GLN | C | 155 | 47.599 | 78.120 | 106.959 | 1.00 | 84.02 | O |
| ATOM | 11406 | N | SER | C | 156 | 49.404 | 78.988 | 105.966 | 1.00 | 87.95 | N |
| ATOM | 11408 | CA | SER | C | 156 | 48.872 | 78.684 | 104.665 | 1.00 | 86.39 | C |
| ATOM | 11410 | CB | SER | C | 156 | 47.818 | 79.707 | 104.245 | 1.00 | 89.74 | C |
| ATOM | 11413 | OG | SER | C | 156 | 48.427 | 80.797 | 103.581 | 1.00 | 95.70 | O |
| ATOM | 11415 | C | SER | C | 156 | 50.039 | 78.564 | 103.698 | 1.00 | 86.84 | C |
| ATOM | 11416 | O | SER | C | 156 | 51.075 | 79.207 | 103.873 | 1.00 | 88.72 | O |
| ATOM | 11417 | N | GLY | C | 157 | 49.867 | 77.656 | 102.735 | 1.00 | 84.26 | N |
| ATOM | 11419 | CA | GLY | C | 157 | 50.836 | 77.421 | 101.684 | 1.00 | 85.21 | C |
| ATOM | 11422 | C | GLY | C | 157 | 52.001 | 76.541 | 102.070 | 1.00 | 82.75 | C |
| ATOM | 11423 | O | GLY | C | 157 | 52.857 | 76.304 | 101.247 | 1.00 | 83.43 | O |
| ATOM | 11424 | N | ASN | C | 158 | 52.019 | 76.050 | 103.302 | 1.00 | 80.64 | N |
| ATOM | 11426 | CA | ASN | C | 158 | 53.087 | 75.180 | 103.820 | 1.00 | 79.04 | C |
| ATOM | 11428 | CB | ASN | C | 158 | 53.786 | 75.891 | 104.976 | 1.00 | 81.79 | C |
| ATOM | 11431 | CG | ASN | C | 158 | 52.797 | 76.357 | 106.077 | 1.00 | 84.07 | C |
| ATOM | 11432 | OD1 | ASN | C | 158 | 51.593 | 76.497 | 105.840 | 1.00 | 85.42 | O |
| ATOM | 11433 | ND2 | ASN | C | 158 | 53.315 | 76.578 | 107.292 | 1.00 | 87.62 | N |
| ATOM | 11436 | C | ASN | C | 158 | 52.554 | 73.770 | 104.259 | 1.00 | 74.13 | C |
| ATOM | 11437 | O | ASN | C | 158 | 53.185 | 73.076 | 105.074 | 1.00 | 72.30 | O |
| ATOM | 11438 | N | SER | C | 159 | 51.392 | 73.365 | 103.719 | 1.00 | 71.66 | N |
| ATOM | 11440 | CA | SER | C | 159 | 50.788 | 72.052 | 104.073 | 1.00 | 67.90 | C |
| ATOM | 11442 | CB | SER | C | 159 | 49.729 | 72.128 | 105.214 | 1.00 | 66.13 | C |
| ATOM | 11445 | OG | SER | C | 159 | 48.749 | 73.114 | 105.020 | 1.00 | 64.64 | O |
| ATOM | 11447 | C | SER | C | 159 | 50.183 | 71.335 | 102.905 | 1.00 | 66.31 | C |
| ATOM | 11448 | O | SER | C | 159 | 49.609 | 71.959 | 102.030 | 1.00 | 68.14 | O |
| ATOM | 11449 | N | GLN | C | 160 | 50.291 | 70.012 | 102.916 | 1.00 | 64.19 | N |
| ATOM | 11451 | CA | GLN | C | 160 | 49.644 | 69.174 | 101.896 | 1.00 | 62.64 | C |
| ATOM | 11453 | CB | GLN | C | 160 | 50.677 | 68.532 | 100.993 | 1.00 | 63.54 | C |
| ATOM | 11456 | CG | GLN | C | 160 | 51.374 | 69.456 | 100.096 | 1.00 | 65.64 | C |
| ATOM | 11459 | CD | GLN | C | 160 | 52.390 | 68.731 | 99.259 | 1.00 | 68.21 | C |
| ATOM | 11460 | OE1 | GLN | C | 160 | 52.193 | 68.544 | 98.030 | 1.00 | 70.43 | O |
| ATOM | 11461 | NE2 | GLN | C | 160 | 53.480 | 68.295 | 99.904 | 1.00 | 66.25 | N |
| ATOM | 11464 | C | GLN | C | 160 | 48.905 | 68.054 | 102.548 | 1.00 | 59.52 | C |
| ATOM | 11465 | O | GLN | C | 160 | 49.353 | 67.499 | 103.546 | 1.00 | 58.40 | O |
| ATOM | 11466 | N | GLU | C | 161 | 47.794 | 67.673 | 101.945 | 1.00 | 58.74 | N |
| ATOM | 11468 | CA | GLU | C | 161 | 47.020 | 66.559 | 102.487 | 1.00 | 56.88 | C |
| ATOM | 11470 | CB | GLU | C | 161 | 45.748 | 67.079 | 103.199 | 1.00 | 57.07 | C |
| ATOM | 11473 | CG | GLU | C | 161 | 44.936 | 68.196 | 102.543 | 1.00 | 59.02 | C |
| ATOM | 11476 | CD | GLU | C | 161 | 43.833 | 68.709 | 103.487 | 1.00 | 61.54 | C |
| ATOM | 11477 | OE1 | GLU | C | 161 | 44.130 | 69.229 | 104.606 | 1.00 | 63.55 | O |
| ATOM | 11478 | OE2 | GLU | C | 161 | 42.647 | 68.567 | 103.133 | 1.00 | 61.99 | O |
| ATOM | 11479 | C | GLU | C | 161 | 46.653 | 65.492 | 101.497 | 1.00 | 55.11 | C |
| ATOM | 11480 | O | GLU | C | 161 | 46.532 | 65.801 | 100.352 | 1.00 | 55.99 | O |
| ATOM | 11481 | N | SER | C | 162 | 46.556 | 64.230 | 101.937 | 1.00 | 53.99 | N |
| ATOM | 11483 | CA | SER | C | 162 | 45.689 | 63.253 | 101.252 | 1.00 | 54.45 | C |
| ATOM | 11485 | CB | SER | C | 162 | 46.338 | 61.980 | 100.687 | 1.00 | 54.88 | C |
| ATOM | 11488 | OG | SER | C | 162 | 47.640 | 61.877 | 100.980 | 1.00 | 54.80 | O |
| ATOM | 11490 | C | SER | C | 162 | 44.599 | 62.743 | 102.111 | 1.00 | 53.51 | C |
| ATOM | 11491 | O | SER | C | 162 | 44.721 | 62.768 | 103.346 | 1.00 | 53.28 | O |
| ATOM | 11492 | N | VAL | C | 163 | 43.602 | 62.185 | 101.394 | 1.00 | 53.67 | N |
| ATOM | 11494 | CA | VAL | C | 163 | 42.504 | 61.396 | 101.915 | 1.00 | 52.73 | C |
| ATOM | 11496 | CB | VAL | C | 163 | 41.162 | 61.948 | 101.478 | 1.00 | 52.14 | C |
| ATOM | 11498 | CG1 | VAL | C | 163 | 40.071 | 61.623 | 102.477 | 1.00 | 52.07 | C |
| ATOM | 11502 | CG2 | VAL | C | 163 | 41.242 | 63.407 | 101.328 | 1.00 | 53.45 | C |
| ATOM | 11506 | C | VAL | C | 163 | 42.564 | 60.049 | 101.289 | 1.00 | 54.59 | C |
| ATOM | 11507 | O | VAL | C | 163 | 43.085 | 59.875 | 100.212 | 1.00 | 56.88 | O |
| ATOM | 11508 | N | THR | C | 164 | 41.975 | 59.088 | 101.948 | 1.00 | 55.77 | N |
| ATOM | 11510 | CA | THR | C | 164 | 41.851 | 57.795 | 101.389 | 1.00 | 58.56 | C |
| ATOM | 11512 | CB | THR | C | 164 | 41.900 | 56.737 | 102.484 | 1.00 | 59.66 | C |
| ATOM | 11514 | OG1 | THR | C | 164 | 42.914 | 57.079 | 103.426 | 1.00 | 59.99 | O |
| ATOM | 11516 | CG2 | THR | C | 164 | 42.467 | 55.366 | 101.889 | 1.00 | 64.68 | C |
| ATOM | 11520 | C | THR | C | 164 | 40.566 | 57.687 | 100.624 | 1.00 | 59.35 | C |
| ATOM | 11521 | O | THR | C | 164 | 39.597 | 58.310 | 100.908 | 1.00 | 58.00 | O |
| ATOM | 11522 | N | GLU | C | 165 | 40.579 | 56.823 | 99.639 | 1.00 | 62.70 | N |
| ATOM | 11524 | CA | GLU | C | 165 | 39.368 | 56.246 | 99.108 | 1.00 | 63.98 | C |
| ATOM | 11526 | CB | GLU | C | 165 | 39.764 | 55.246 | 98.035 | 1.00 | 68.42 | C |
| ATOM | 11529 | CG | GLU | C | 165 | 40.394 | 55.913 | 96.779 | 1.00 | 74.03 | C |
| ATOM | 11532 | CD | GLU | C | 165 | 39.509 | 57.028 | 96.100 | 1.00 | 78.67 | C |
| ATOM | 11533 | OE1 | GLU | C | 165 | 38.238 | 56.972 | 96.180 | 1.00 | 81.40 | O |
| ATOM | 11534 | OE2 | GLU | C | 165 | 40.078 | 57.978 | 95.467 | 1.00 | 80.96 | O |
| ATOM | 11535 | C | GLU | C | 165 | 38.537 | 55.561 | 100.192 | 1.00 | 62.40 | C |
| ATOM | 11536 | O | GLU | C | 165 | 39.094 | 55.035 | 101.158 | 1.00 | 62.62 | O |
| ATOM | 11537 | N | GLN | C | 166 | 37.206 | 55.570 | 100.001 | 1.00 | 61.60 | N |
| ATOM | 11539 | CA | GLN | C | 166 | 36.190 | 55.023 | 100.958 | 1.00 | 60.28 | C |
| ATOM | 11541 | CB | GLN | C | 166 | 34.789 | 55.255 | 100.390 | 1.00 | 60.22 | C |
| ATOM | 11544 | CG | GLN | C | 166 | 33.671 | 54.930 | 101.352 | 1.00 | 60.01 | C |
| ATOM | 11547 | CD | GLN | C | 166 | 32.343 | 55.571 | 101.001 | 1.00 | 57.99 | C |
| ATOM | 11548 | OE1 | GLN | C | 166 | 31.947 | 55.629 | 99.846 | 1.00 | 60.56 | O |
| ATOM | 11549 | NE2 | GLN | C | 166 | 31.631 | 55.984 | 102.008 | 1.00 | 56.87 | N |
| ATOM | 11552 | C | GLN | C | 166 | 36.369 | 53.544 | 101.355 | 1.00 | 61.82 | C |
| ATOM | 11553 | O | GLN | C | 166 | 36.668 | 52.732 | 100.536 | 1.00 | 63.96 | O |
| ATOM | 11554 | N | ASP | C | 167 | 36.172 | 53.205 | 102.611 | 1.00 | 61.44 | N |
| ATOM | 11556 | CA | ASP | C | 167 | 36.546 | 51.900 | 103.107 | 1.00 | 65.73 | C |
| ATOM | 11558 | CB | ASP | C | 167 | 36.746 | 51.988 | 104.586 | 1.00 | 65.19 | C |
| ATOM | 11561 | CG | ASP | C | 167 | 37.345 | 50.724 | 105.181 | 1.00 | 72.04 | C |
| ATOM | 11562 | OD1 | ASP | C | 167 | 36.669 | 49.640 | 105.326 | 1.00 | 75.74 | O |
| ATOM | 11563 | OD2 | ASP | C | 167 | 38.529 | 50.768 | 105.587 | 1.00 | 76.75 | O |
| ATOM | 11564 | C | ASP | C | 167 | 35.428 | 50.923 | 102.904 | 1.00 | 69.41 | C |
| ATOM | 11565 | O | ASP | C | 167 | 34.325 | 51.175 | 103.348 | 1.00 | 68.59 | O |
| ATOM | 11566 | N | SER | C | 168 | 35.692 | 49.784 | 102.272 | 1.00 | 74.58 | N |
| ATOM | 11568 | CA | SER | C | 168 | 34.612 | 48.829 | 101.949 | 1.00 | 78.10 | C |
| ATOM | 11570 | CB | SER | C | 168 | 35.157 | 47.618 | 101.188 | 1.00 | 83.31 | C |
| ATOM | 11573 | OG | SER | C | 168 | 36.431 | 47.235 | 101.689 | 1.00 | 84.86 | O |
| ATOM | 11575 | C | SER | C | 168 | 33.831 | 48.435 | 103.227 | 1.00 | 78.77 | C |
| ATOM | 11576 | O | SER | C | 168 | 32.616 | 48.748 | 103.318 | 1.00 | 78.19 | O |
| ATOM | 11577 | N | LYS | C | 169 | 34.533 | 47.857 | 104.227 | 1.00 | 80.20 | N |
| ATOM | 11579 | CA | LYS | C | 169 | 33.923 | 47.466 | 105.543 | 1.00 | 81.36 | C |
| ATOM | 11581 | CB | LYS | C | 169 | 34.976 | 46.844 | 106.508 | 1.00 | 84.06 | C |
| ATOM | 11584 | CG | LYS | C | 169 | 35.667 | 45.526 | 106.043 | 1.00 | 90.82 | C |
| ATOM | 11587 | CD | LYS | C | 169 | 37.176 | 45.430 | 106.502 | 1.00 | 92.25 | C |
| ATOM | 11590 | CE | LYS | C | 169 | 37.919 | 44.219 | 105.925 | 1.00 | 98.14 | C |
| ATOM | 11593 | NZ | LYS | C | 169 | 37.925 | 43.042 | 106.880 | 1.00 | 104.47 | N |
| ATOM | 11597 | C | LYS | C | 169 | 33.080 | 48.578 | 106.300 | 1.00 | 76.62 | C |
| ATOM | 11598 | O | LYS | C | 169 | 31.880 | 48.420 | 106.429 | 1.00 | 77.24 | O |
| ATOM | 11599 | N | ASP | C | 170 | 33.703 | 49.652 | 106.806 | 1.00 | 72.36 | N |
| ATOM | 11601 | CA | ASP | C | 170 | 32.992 | 50.745 | 107.525 | 1.00 | 68.94 | C |
| ATOM | 11603 | CB | ASP | C | 170 | 33.753 | 51.190 | 108.779 | 1.00 | 68.28 | C |
| ATOM | 11606 | CG | ASP | C | 170 | 35.172 | 51.717 | 108.499 | 1.00 | 68.33 | C |
| ATOM | 11607 | OD1 | ASP | C | 170 | 35.669 | 51.859 | 107.357 | 1.00 | 68.00 | O |
| ATOM | 11608 | OD2 | ASP | C | 170 | 35.903 | 52.067 | 109.425 | 1.00 | 71.49 | O |
| ATOM | 11609 | C | ASP | C | 170 | 32.608 | 51.977 | 106.662 | 1.00 | 65.16 | C |
| ATOM | 11610 | O | ASP | C | 170 | 32.043 | 52.960 | 107.137 | 1.00 | 61.37 | O |
| ATOM | 11611 | N | SER | C | 171 | 32.914 | 51.881 | 105.374 | 1.00 | 65.83 | N |
| ATOM | 11613 | CA | SER | C | 171 | 32.547 | 52.884 | 104.369 | 1.00 | 63.62 | C |
| ATOM | 11615 | CB | SER | C | 171 | 31.104 | 52.585 | 103.921 | 1.00 | 64.50 | C |
| ATOM | 11618 | OG | SER | C | 171 | 30.278 | 53.545 | 104.481 | 1.00 | 62.30 | O |
| ATOM | 11620 | C | SER | C | 171 | 32.857 | 54.392 | 104.719 | 1.00 | 59.93 | C |
| ATOM | 11621 | O | SER | C | 171 | 32.140 | 55.332 | 104.368 | 1.00 | 58.66 | O |
| ATOM | 11622 | N | THR | C | 172 | 34.017 | 54.602 | 105.330 | 1.00 | 59.37 | N |
| ATOM | 11624 | CA | THR | C | 172 | 34.528 | 55.938 | 105.707 | 1.00 | 55.85 | C |
| ATOM | 11626 | CB | THR | C | 172 | 34.674 | 55.962 | 107.198 | 1.00 | 55.89 | C |
| ATOM | 11628 | OG1 | THR | C | 172 | 35.487 | 57.071 | 107.513 | 1.00 | 59.13 | O |
| ATOM | 11630 | CG2 | THR | C | 172 | 35.511 | 54.826 | 107.712 | 1.00 | 57.10 | C |
| ATOM | 11634 | C | THR | C | 172 | 35.897 | 56.403 | 105.084 | 1.00 | 53.92 | C |
| ATOM | 11635 | O | THR | C | 172 | 36.617 | 55.676 | 104.436 | 1.00 | 54.08 | O |
| ATOM | 11636 | N | TYR | C | 173 | 36.228 | 57.655 | 105.324 | 1.00 | 52.30 | N |
| ATOM | 11638 | CA | TYR | C | 173 | 37.479 | 58.291 | 104.859 | 1.00 | 51.46 | C |
| ATOM | 11640 | CB | TYR | C | 173 | 37.166 | 59.643 | 104.193 | 1.00 | 49.71 | C |
| ATOM | 11643 | CG | TYR | C | 173 | 36.218 | 59.481 | 103.061 | 1.00 | 49.79 | C |
| ATOM | 11644 | CD1 | TYR | C | 173 | 34.872 | 59.517 | 103.254 | 1.00 | 49.00 | C |
| ATOM | 11646 | CE1 | TYR | C | 173 | 34.033 | 59.333 | 102.222 | 1.00 | 52.19 | C |
| ATOM | 11648 | CZ | TYR | C | 173 | 34.545 | 59.096 | 100.980 | 1.00 | 53.46 | C |
| ATOM | 11649 | OH | TYR | C | 173 | 33.727 | 58.910 | 99.884 | 1.00 | 58.02 | O |
| ATOM | 11651 | CE2 | TYR | C | 173 | 35.868 | 59.059 | 100.794 | 1.00 | 52.66 | C |
| ATOM | 11653 | CD2 | TYR | C | 173 | 36.680 | 59.236 | 101.810 | 1.00 | 51.00 | C |
| ATOM | 11655 | C | TYR | C | 173 | 38.513 | 58.493 | 105.961 | 1.00 | 51.35 | C |
| ATOM | 11656 | O | TYR | C | 173 | 38.240 | 58.326 | 107.138 | 1.00 | 51.36 | O |
| ATOM | 11657 | N | SER | C | 174 | 39.701 | 58.907 | 105.547 | 1.00 | 51.83 | N |
| ATOM | 11659 | CA | SER | C | 174 | 40.799 | 59.144 | 106.484 | 1.00 | 52.31 | C |
| ATOM | 11661 | CB | SER | C | 174 | 41.538 | 57.809 | 106.747 | 1.00 | 54.03 | C |
| ATOM | 11664 | OG | SER | C | 174 | 41.293 | 57.287 | 108.023 | 1.00 | 52.58 | O |
| ATOM | 11666 | C | SER | C | 174 | 41.742 | 60.193 | 105.881 | 1.00 | 51.52 | C |
| ATOM | 11667 | O | SER | C | 174 | 42.203 | 60.034 | 104.801 | 1.00 | 52.07 | O |
| ATOM | 11668 | N | LEU | C | 175 | 42.048 | 61.249 | 106.587 | 1.00 | 51.86 | N |
| ATOM | 11670 | CA | LEU | C | 175 | 42.874 | 62.315 | 106.019 | 1.00 | 52.36 | C |
| ATOM | 11672 | CB | LEU | C | 175 | 42.244 | 63.649 | 106.309 | 1.00 | 51.94 | C |
| ATOM | 11675 | CG | LEU | C | 175 | 42.542 | 64.815 | 105.400 | 1.00 | 51.62 | C |
| ATOM | 11677 | CD1 | LEU | C | 175 | 41.344 | 65.221 | 104.572 | 1.00 | 50.59 | C |
| ATOM | 11681 | CD2 | LEU | C | 175 | 42.946 | 65.915 | 106.230 | 1.00 | 52.90 | C |
| ATOM | 11685 | C | LEU | C | 175 | 44.220 | 62.353 | 106.658 | 1.00 | 54.29 | C |
| ATOM | 11686 | O | LEU | C | 175 | 44.426 | 61.809 | 107.718 | 1.00 | 55.77 | O |
| ATOM | 11687 | N | SER | C | 176 | 45.160 | 63.032 | 106.029 | 1.00 | 55.63 | N |
| ATOM | 11689 | CA | SER | C | 176 | 46.416 | 63.293 | 106.700 | 1.00 | 56.66 | C |
| ATOM | 11691 | CB | SER | C | 176 | 47.413 | 62.207 | 106.400 | 1.00 | 57.54 | C |
| ATOM | 11694 | OG | SER | C | 176 | 48.503 | 62.771 | 105.728 | 1.00 | 61.11 | O |
| ATOM | 11696 | C | SER | C | 176 | 46.915 | 64.623 | 106.226 | 1.00 | 57.20 | C |
| ATOM | 11697 | O | SER | C | 176 | 46.943 | 64.902 | 105.017 | 1.00 | 56.36 | O |
| ATOM | 11698 | N | SER | C | 177 | 47.292 | 65.468 | 107.193 | 1.00 | 58.72 | N |
| ATOM | 11700 | CA | SER | C | 177 | 47.898 | 66.776 | 106.878 | 1.00 | 59.21 | C |
| ATOM | 11702 | CB | SER | C | 177 | 47.171 | 67.915 | 107.543 | 1.00 | 59.79 | C |
| ATOM | 11705 | OG | SER | C | 177 | 47.283 | 69.028 | 106.687 | 1.00 | 61.45 | O |
| ATOM | 11707 | C | SER | C | 177 | 49.295 | 66.763 | 107.325 | 1.00 | 59.52 | C |
| ATOM | 11708 | O | SER | C | 177 | 49.564 | 66.372 | 108.408 | 1.00 | 59.47 | O |
| ATOM | 11709 | N | THR | C | 178 | 50.183 | 67.147 | 106.438 | 1.00 | 60.76 | N |
| ATOM | 11711 | CA | THR | C | 178 | 51.600 | 67.208 | 106.743 | 1.00 | 62.63 | C |
| ATOM | 11713 | CB | THR | C | 178 | 52.400 | 66.234 | 105.865 | 1.00 | 62.33 | C |
| ATOM | 11715 | OG1 | THR | C | 178 | 52.028 | 64.884 | 106.194 | 1.00 | 62.25 | O |
| ATOM | 11717 | CG2 | THR | C | 178 | 53.836 | 66.288 | 106.225 | 1.00 | 63.69 | C |
| ATOM | 11721 | C | THR | C | 178 | 52.063 | 68.655 | 106.630 | 1.00 | 64.52 | C |
| ATOM | 11722 | O | THR | C | 178 | 52.212 | 69.231 | 105.532 | 1.00 | 65.27 | O |
| ATOM | 11723 | N | LEU | C | 179 | 52.186 | 69.261 | 107.800 | 1.00 | 66.36 | N |
| ATOM | 11725 | CA | LEU | C | 179 | 52.856 | 70.552 | 107.944 | 1.00 | 69.87 | C |
| ATOM | 11727 | CB | LEU | C | 179 | 52.540 | 71.183 | 109.294 | 1.00 | 71.47 | C |
| ATOM | 11730 | CG | LEU | C | 179 | 53.257 | 72.492 | 109.522 | 1.00 | 75.84 | C |
| ATOM | 11732 | CD1 | LEU | C | 179 | 52.876 | 73.551 | 108.468 | 1.00 | 78.63 | C |
| ATOM | 11736 | CD2 | LEU | C | 179 | 52.886 | 72.972 | 110.888 | 1.00 | 79.77 | C |
| ATOM | 11740 | C | LEU | C | 179 | 54.370 | 70.431 | 107.757 | 1.00 | 71.38 | C |
| ATOM | 11741 | O | LEU | C | 179 | 55.035 | 69.600 | 108.367 | 1.00 | 71.08 | O |
| ATOM | 11742 | N | THR | C | 180 | 54.892 | 71.257 | 106.871 | 1.00 | 73.42 | N |
| ATOM | 11744 | CA | THR | C | 180 | 56.279 | 71.148 | 106.515 | 1.00 | 75.49 | C |
| ATOM | 11746 | CB | THR | C | 180 | 56.400 | 70.535 | 105.098 | 1.00 | 74.91 | C |
| ATOM | 11748 | OG1 | THR | C | 180 | 55.394 | 69.499 | 104.878 | 1.00 | 70.42 | O |
| ATOM | 11750 | CG2 | THR | C | 180 | 57.757 | 69.818 | 104.953 | 1.00 | 76.14 | C |
| ATOM | 11754 | C | THR | C | 180 | 57.016 | 72.488 | 106.671 | 1.00 | 79.35 | C |
| ATOM | 11755 | O | THR | C | 180 | 56.499 | 73.558 | 106.316 | 1.00 | 80.35 | O |
| ATOM | 11756 | N | LEU | C | 181 | 58.218 | 72.376 | 107.238 | 1.00 | 81.53 | N |
| ATOM | 11758 | CA | LEU | C | 181 | 59.046 | 73.493 | 107.698 | 1.00 | 86.10 | C |
| ATOM | 11760 | CB | LEU | C | 181 | 58.626 | 73.845 | 109.107 | 1.00 | 87.52 | C |
| ATOM | 11763 | CG | LEU | C | 181 | 57.270 | 74.495 | 109.258 | 1.00 | 88.57 | C |
| ATOM | 11765 | CD1 | LEU | C | 181 | 57.123 | 74.780 | 110.767 | 1.00 | 91.29 | C |
| ATOM | 11769 | CD2 | LEU | C | 181 | 57.124 | 75.802 | 108.372 | 1.00 | 92.28 | C |
| ATOM | 11773 | C | LEU | C | 181 | 60.531 | 73.127 | 107.790 | 1.00 | 87.89 | C |
| ATOM | 11774 | O | LEU | C | 181 | 60.834 | 72.002 | 108.185 | 1.00 | 87.01 | O |
| ATOM | 11775 | N | SER | C | 182 | 61.451 | 74.058 | 107.498 | 1.00 | 90.99 | N |
| ATOM | 11777 | CA | SER | C | 182 | 62.872 | 73.750 | 107.637 | 1.00 | 92.84 | C |
| ATOM | 11779 | CB | SER | C | 182 | 63.746 | 74.837 | 107.014 | 1.00 | 96.46 | C |
| ATOM | 11782 | OG | SER | C | 182 | 64.103 | 75.790 | 107.977 | 1.00 | 99.90 | O |
| ATOM | 11784 | C | SER | C | 182 | 63.126 | 73.614 | 109.129 | 1.00 | 94.76 | C |
| ATOM | 11785 | O | SER | C | 182 | 62.526 | 74.368 | 109.888 | 1.00 | 96.46 | O |
| ATOM | 11786 | N | LYS | C | 183 | 63.973 | 72.665 | 109.562 | 1.00 | 95.19 | N |
| ATOM | 11788 | CA | LYS | C | 183 | 64.345 | 72.530 | 111.001 | 1.00 | 97.89 | C |
| ATOM | 11790 | CB | LYS | C | 183 | 65.603 | 71.666 | 111.218 | 1.00 | 99.64 | C |
| ATOM | 11797 | C | LYS | C | 183 | 64.651 | 73.848 | 111.670 | 1.00 | 102.52 | C |
| ATOM | 11798 | O | LYS | C | 183 | 64.684 | 73.922 | 112.904 | 1.00 | 105.22 | O |
| ATOM | 11799 | N | ALA | C | 184 | 64.965 | 74.851 | 110.850 | 1.00 | 104.32 | N |
| ATOM | 11801 | CA | ALA | C | 184 | 65.255 | 76.202 | 111.322 | 1.00 | 109.49 | C |
| ATOM | 11803 | CB | ALA | C | 184 | 65.910 | 76.997 | 110.192 | 1.00 | 111.80 | C |
| ATOM | 11807 | C | ALA | C | 184 | 64.015 | 76.946 | 111.857 | 1.00 | 109.86 | C |
| ATOM | 11808 | O | ALA | C | 184 | 64.043 | 77.545 | 112.937 | 1.00 | 113.60 | O |
| ATOM | 11809 | N | ASP | C | 185 | 62.940 | 76.921 | 111.075 | 1.00 | 106.51 | N |
| ATOM | 11811 | CA | ASP | C | 185 | 61.677 | 77.545 | 111.468 | 1.00 | 106.54 | C |
| ATOM | 11813 | CB | ASP | C | 185 | 60.662 | 77.485 | 110.312 | 1.00 | 103.12 | C |
| ATOM | 11816 | CG | ASP | C | 185 | 61.105 | 78.269 | 109.052 | 1.00 | 106.46 | C |
| ATOM | 11817 | OD1 | ASP | C | 185 | 61.944 | 79.204 | 109.095 | 1.00 | 110.53 | O |
| ATOM | 11818 | OD2 | ASP | C | 185 | 60.608 | 78.007 | 107.935 | 1.00 | 108.38 | O |
| ATOM | 11819 | C | ASP | C | 185 | 61.087 | 76.833 | 112.689 | 1.00 | 104.72 | C |
| ATOM | 11820 | O | ASP | C | 185 | 60.448 | 77.451 | 113.547 | 1.00 | 107.05 | O |
| ATOM | 11821 | N | TYR | C | 186 | 61.307 | 75.525 | 112.758 | 1.00 | 100.91 | N |
| ATOM | 11823 | CA | TYR | C | 186 | 60.650 | 74.704 | 113.761 | 1.00 | 99.44 | C |
| ATOM | 11825 | CB | TYR | C | 186 | 60.821 | 73.215 | 113.428 | 1.00 | 95.52 | C |
| ATOM | 11828 | CG | TYR | C | 186 | 60.182 | 72.269 | 114.423 | 1.00 | 94.50 | C |
| ATOM | 11829 | CD1 | TYR | C | 186 | 58.820 | 72.302 | 114.676 | 1.00 | 92.95 | C |
| ATOM | 11831 | CE1 | TYR | C | 186 | 58.228 | 71.416 | 115.611 | 1.00 | 92.00 | C |
| ATOM | 11833 | CZ | TYR | C | 186 | 59.018 | 70.511 | 116.287 | 1.00 | 92.21 | C |
| ATOM | 11834 | OH | TYR | C | 186 | 58.475 | 69.656 | 117.188 | 1.00 | 90.68 | O |
| ATOM | 11836 | CE2 | TYR | C | 186 | 60.364 | 70.460 | 116.052 | 1.00 | 95.55 | C |
| ATOM | 11838 | CD2 | TYR | C | 186 | 60.948 | 71.339 | 115.126 | 1.00 | 96.74 | C |
| ATOM | 11840 | C | TYR | C | 186 | 61.198 | 75.043 | 115.143 | 1.00 | 104.67 | C |
| ATOM | 11841 | O | TYR | C | 186 | 60.414 | 75.165 | 116.098 | 1.00 | 106.10 | O |
| ATOM | 11842 | N | GLU | C | 187 | 62.532 | 75.225 | 115.226 | 1.00 | 107.76 | N |
| ATOM | 11844 | CA | GLU | C | 187 | 63.227 | 75.577 | 116.466 | 1.00 | 112.95 | C |
| ATOM | 11846 | CB | GLU | C | 187 | 64.703 | 75.893 | 116.196 | 1.00 | 115.75 | C |
| ATOM | 11853 | C | GLU | C | 187 | 62.516 | 76.763 | 117.096 | 1.00 | 116.87 | C |
| ATOM | 11854 | O | GLU | C | 187 | 62.133 | 76.705 | 118.270 | 1.00 | 119.20 | O |
| ATOM | 11855 | N | LYS | C | 188 | 62.254 | 77.785 | 116.272 | 1.00 | 117.68 | N |
| ATOM | 11857 | CA | LYS | C | 188 | 61.806 | 79.119 | 116.728 | 1.00 | 123.34 | C |
| ATOM | 11859 | CB | LYS | C | 188 | 62.258 | 80.186 | 115.693 | 1.00 | 125.15 | C |
| ATOM | 11866 | C | LYS | C | 188 | 60.293 | 79.334 | 117.137 | 1.00 | 122.83 | C |
| ATOM | 11867 | O | LYS | C | 188 | 59.870 | 80.480 | 117.337 | 1.00 | 127.37 | O |
| ATOM | 11868 | N | HIS | C | 189 | 59.498 | 78.271 | 117.295 | 1.00 | 118.14 | N |
| ATOM | 11870 | CA | HIS | C | 189 | 58.118 | 78.401 | 117.838 | 1.00 | 118.23 | C |
| ATOM | 11872 | CB | HIS | C | 189 | 57.039 | 78.402 | 116.701 | 1.00 | 113.37 | C |
| ATOM | 11875 | CG | HIS | C | 189 | 57.197 | 79.528 | 115.718 | 1.00 | 115.83 | C |
| ATOM | 11876 | ND1 | HIS | C | 189 | 56.933 | 80.841 | 116.040 | 1.00 | 122.53 | N |
| ATOM | 11878 | CE1 | HIS | C | 189 | 57.218 | 81.617 | 115.011 | 1.00 | 123.27 | C |
| ATOM | 11880 | NE2 | HIS | C | 189 | 57.650 | 80.854 | 114.025 | 1.00 | 118.61 | N |
| ATOM | 11882 | CD2 | HIS | C | 189 | 57.647 | 79.542 | 114.440 | 1.00 | 114.31 | C |
| ATOM | 11884 | C | HIS | C | 189 | 57.835 | 77.295 | 118.865 | 1.00 | 116.94 | C |
| ATOM | 11885 | O | HIS | C | 189 | 58.396 | 76.204 | 118.757 | 1.00 | 114.30 | O |
| ATOM | 11886 | N | LYS | C | 190 | 56.965 | 77.557 | 119.843 | 1.00 | 119.39 | N |
| ATOM | 11888 | CA | LYS | C | 190 | 56.663 | 76.554 | 120.870 | 1.00 | 119.33 | C |
| ATOM | 11890 | CB | LYS | C | 190 | 56.311 | 77.194 | 122.220 | 1.00 | 126.70 | C |
| ATOM | 11897 | C | LYS | C | 190 | 55.537 | 75.659 | 120.451 | 1.00 | 113.38 | C |
| ATOM | 11898 | O | LYS | C | 190 | 55.777 | 74.497 | 120.119 | 1.00 | 109.64 | O |
| ATOM | 11899 | N | VAL | C | 191 | 54.318 | 76.222 | 120.457 | 1.00 | 113.48 | N |
| ATOM | 11901 | CA | VAL | C | 191 | 53.055 | 75.504 | 120.222 | 1.00 | 108.33 | C |
| ATOM | 11903 | CB | VAL | C | 191 | 51.789 | 76.364 | 120.601 | 1.00 | 110.82 | C |
| ATOM | 11905 | CG1 | VAL | C | 191 | 50.492 | 75.634 | 120.279 | 1.00 | 106.29 | C |
| ATOM | 11909 | CG2 | VAL | C | 191 | 51.761 | 76.701 | 122.048 | 1.00 | 117.81 | C |
| ATOM | 11913 | C | VAL | C | 191 | 52.932 | 75.172 | 118.762 | 1.00 | 102.06 | C |
| ATOM | 11914 | O | VAL | C | 191 | 53.386 | 75.958 | 117.927 | 1.00 | 101.58 | O |
| ATOM | 11915 | N | TYR | C | 192 | 52.284 | 74.024 | 118.493 | 1.00 | 97.43 | N |
| ATOM | 11917 | CA | TYR | C | 192 | 51.969 | 73.511 | 117.156 | 1.00 | 91.95 | C |
| ATOM | 11919 | CB | TYR | C | 192 | 53.046 | 72.561 | 116.731 | 1.00 | 89.41 | C |
| ATOM | 11922 | CG | TYR | C | 192 | 54.216 | 73.257 | 116.212 | 1.00 | 90.56 | C |
| ATOM | 11923 | CD1 | TYR | C | 192 | 55.338 | 73.417 | 116.990 | 1.00 | 97.01 | C |
| ATOM | 11925 | CE1 | TYR | C | 192 | 56.440 | 74.109 | 116.506 | 1.00 | 99.63 | C |
| ATOM | 11927 | CZ | TYR | C | 192 | 56.404 | 74.640 | 115.220 | 1.00 | 96.17 | C |
| ATOM | 11928 | OH | TYR | C | 192 | 57.487 | 75.340 | 114.715 | 1.00 | 99.78 | O |
| ATOM | 11930 | CE2 | TYR | C | 192 | 55.283 | 74.479 | 114.450 | 1.00 | 90.48 | C |
| ATOM | 11932 | CD2 | TYR | C | 192 | 54.194 | 73.817 | 114.961 | 1.00 | 87.27 | C |
| ATOM | 11934 | C | TYR | C | 192 | 50.653 | 72.713 | 117.135 | 1.00 | 89.66 | C |
| ATOM | 11935 | O | TYR | C | 192 | 50.545 | 71.644 | 117.772 | 1.00 | 89.20 | O |
| ATOM | 11936 | N | ALA | C | 193 | 49.684 | 73.192 | 116.352 | 1.00 | 88.28 | N |
| ATOM | 11938 | CA | ALA | C | 193 | 48.281 | 72.900 | 116.635 | 1.00 | 87.68 | C |
| ATOM | 11940 | CB | ALA | C | 193 | 47.569 | 74.139 | 117.180 | 1.00 | 91.96 | C |
| ATOM | 11944 | C | ALA | C | 193 | 47.588 | 72.431 | 115.403 | 1.00 | 83.46 | C |
| ATOM | 11945 | O | ALA | C | 193 | 47.920 | 72.887 | 114.312 | 1.00 | 83.57 | O |
| ATOM | 11946 | N | CYS | C | 194 | 46.602 | 71.551 | 115.588 | 1.00 | 81.07 | N |
| ATOM | 11948 | CA | CYS | C | 194 | 45.912 | 70.929 | 114.502 | 1.00 | 76.76 | C |
| ATOM | 11950 | CB | CYS | C | 194 | 46.244 | 69.455 | 114.509 | 1.00 | 74.32 | C |
| ATOM | 11953 | SG | CYS | C | 194 | 45.544 | 68.692 | 113.051 | 1.00 | 76.90 | S |
| ATOM | 11954 | C | CYS | C | 194 | 44.444 | 71.168 | 114.783 | 1.00 | 76.26 | C |
| ATOM | 11955 | O | CYS | C | 194 | 43.916 | 70.603 | 115.720 | 1.00 | 77.18 | O |
| ATOM | 11956 | N | GLU | C | 195 | 43.790 | 72.021 | 113.991 | 1.00 | 75.58 | N |
| ATOM | 11958 | CA | GLU | C | 195 | 42.376 | 72.394 | 114.203 | 1.00 | 75.90 | C |
| ATOM | 11960 | CB | GLU | C | 195 | 42.230 | 73.907 | 114.332 | 1.00 | 80.39 | C |
| ATOM | 11963 | CG | GLU | C | 195 | 40.824 | 74.381 | 114.736 | 1.00 | 83.63 | C |
| ATOM | 11966 | CD | GLU | C | 195 | 40.380 | 75.689 | 114.059 | 1.00 | 87.24 | C |
| ATOM | 11967 | OE1 | GLU | C | 195 | 41.166 | 76.308 | 113.301 | 1.00 | 87.78 | O |
| ATOM | 11968 | OE2 | GLU | C | 195 | 39.230 | 76.118 | 114.284 | 1.00 | 89.97 | O |
| ATOM | 11969 | C | GLU | C | 195 | 41.369 | 71.845 | 113.145 | 1.00 | 71.74 | C |
| ATOM | 11970 | O | GLU | C | 195 | 41.127 | 72.413 | 112.070 | 1.00 | 70.64 | O |
| ATOM | 11971 | N | VAL | C | 196 | 40.744 | 70.741 | 113.531 | 1.00 | 69.55 | N |
| ATOM | 11973 | CA | VAL | C | 196 | 39.805 | 70.023 | 112.708 | 1.00 | 65.72 | C |
| ATOM | 11975 | CB | VAL | C | 196 | 39.792 | 68.572 | 113.122 | 1.00 | 63.49 | C |
| ATOM | 11977 | CG1 | VAL | C | 196 | 38.864 | 67.812 | 112.277 | 1.00 | 60.95 | C |
| ATOM | 11981 | CG2 | VAL | C | 196 | 41.195 | 67.993 | 113.025 | 1.00 | 62.64 | C |
| ATOM | 11985 | C | VAL | C | 196 | 38.388 | 70.591 | 112.830 | 1.00 | 66.93 | C |
| ATOM | 11986 | O | VAL | C | 196 | 37.907 | 70.990 | 113.890 | 1.00 | 68.84 | O |
| ATOM | 11987 | N | THR | C | 197 | 37.727 | 70.587 | 111.693 | 1.00 | 65.22 | N |
| ATOM | 11989 | CA | THR | C | 197 | 36.375 | 71.068 | 111.555 | 1.00 | 66.67 | C |
| ATOM | 11991 | CB | THR | C | 197 | 36.395 | 72.420 | 110.849 | 1.00 | 68.91 | C |
| ATOM | 11993 | OG1 | THR | C | 197 | 37.268 | 73.302 | 111.567 | 1.00 | 73.48 | O |
| ATOM | 11995 | CG2 | THR | C | 197 | 35.026 | 73.088 | 110.862 | 1.00 | 70.65 | C |
| ATOM | 11999 | C | THR | C | 197 | 35.623 | 70.042 | 110.718 | 1.00 | 64.04 | C |
| ATOM | 12000 | O | THR | C | 197 | 36.033 | 69.666 | 109.600 | 1.00 | 62.98 | O |
| ATOM | 12001 | N | HIS | C | 198 | 34.511 | 69.574 | 111.243 | 1.00 | 64.16 | N |
| ATOM | 12003 | CA | HIS | C | 198 | 33.842 | 68.464 | 110.617 | 1.00 | 61.33 | C |
| ATOM | 12005 | CB | HIS | C | 198 | 34.562 | 67.151 | 110.973 | 1.00 | 59.74 | C |
| ATOM | 12008 | CG | HIS | C | 198 | 33.971 | 65.964 | 110.287 | 1.00 | 58.02 | C |
| ATOM | 12009 | ND1 | HIS | C | 198 | 34.128 | 65.750 | 108.936 | 1.00 | 57.39 | N |
| ATOM | 12011 | CE1 | HIS | C | 198 | 33.441 | 64.677 | 108.594 | 1.00 | 56.71 | C |
| ATOM | 12013 | NE2 | HIS | C | 198 | 32.826 | 64.208 | 109.663 | 1.00 | 54.74 | N |
| ATOM | 12015 | CD2 | HIS | C | 198 | 33.140 | 64.993 | 110.736 | 1.00 | 55.73 | C |
| ATOM | 12017 | C | HIS | C | 198 | 32.439 | 68.464 | 111.115 | 1.00 | 62.12 | C |
| ATOM | 12018 | O | HIS | C | 198 | 32.191 | 68.978 | 112.181 | 1.00 | 65.48 | O |
| ATOM | 12019 | N | GLN | C | 199 | 31.529 | 67.905 | 110.337 | 1.00 | 60.45 | N |
| ATOM | 12021 | CA | GLN | C | 199 | 30.113 | 67.879 | 110.673 | 1.00 | 61.89 | C |
| ATOM | 12023 | CB | GLN | C | 199 | 29.321 | 67.205 | 109.531 | 1.00 | 60.84 | C |
| ATOM | 12026 | CG | GLN | C | 199 | 27.791 | 67.469 | 109.465 | 1.00 | 62.62 | C |
| ATOM | 12029 | CD | GLN | C | 199 | 27.118 | 66.758 | 108.272 | 1.00 | 62.17 | C |
| ATOM | 12030 | OE1 | GLN | C | 199 | 27.603 | 66.780 | 107.132 | 1.00 | 62.44 | O |
| ATOM | 12031 | NE2 | GLN | C | 199 | 26.021 | 66.130 | 108.541 | 1.00 | 62.82 | N |
| ATOM | 12034 | C | GLN | C | 199 | 29.881 | 67.134 | 111.977 | 1.00 | 62.72 | C |
| ATOM | 12035 | O | GLN | C | 199 | 28.947 | 67.431 | 112.702 | 1.00 | 64.57 | O |
| ATOM | 12036 | N | GLY | C | 200 | 30.759 | 66.186 | 112.294 | 1.00 | 61.62 | N |
| ATOM | 12038 | CA | GLY | C | 200 | 30.553 | 65.315 | 113.435 | 1.00 | 62.79 | C |
| ATOM | 12041 | C | GLY | C | 200 | 31.172 | 65.785 | 114.730 | 1.00 | 65.81 | C |
| ATOM | 12042 | O | GLY | C | 200 | 31.463 | 64.929 | 115.553 | 1.00 | 68.31 | O |
| ATOM | 12043 | N | LEU | C | 201 | 31.378 | 67.103 | 114.910 | 1.00 | 67.08 | N |
| ATOM | 12045 | CA | LEU | C | 201 | 32.111 | 67.693 | 116.042 | 1.00 | 69.26 | C |
| ATOM | 12047 | CB | LEU | C | 201 | 33.565 | 67.903 | 115.686 | 1.00 | 67.39 | C |
| ATOM | 12050 | CG | LEU | C | 201 | 34.388 | 66.703 | 115.257 | 1.00 | 64.11 | C |
| ATOM | 12052 | CD1 | LEU | C | 201 | 35.552 | 67.128 | 114.421 | 1.00 | 62.77 | C |
| ATOM | 12056 | CD2 | LEU | C | 201 | 34.886 | 65.911 | 116.436 | 1.00 | 66.42 | C |
| ATOM | 12060 | C | LEU | C | 201 | 31.490 | 69.063 | 116.339 | 1.00 | 73.17 | C |
| ATOM | 12061 | O | LEU | C | 201 | 31.436 | 69.928 | 115.455 | 1.00 | 72.65 | O |
| ATOM | 12062 | N | SER | C | 202 | 31.017 | 69.270 | 117.568 | 1.00 | 77.26 | N |
| ATOM | 12064 | CA | SER | C | 202 | 30.015 | 70.328 | 117.787 | 1.00 | 81.12 | C |
| ATOM | 12066 | CB | SER | C | 202 | 29.362 | 70.258 | 119.187 | 1.00 | 85.90 | C |
| ATOM | 12069 | OG | SER | C | 202 | 29.672 | 69.042 | 119.838 | 1.00 | 85.41 | O |
| ATOM | 12071 | C | SER | C | 202 | 30.667 | 71.668 | 117.561 | 1.00 | 82.89 | C |
| ATOM | 12072 | O | SER | C | 202 | 30.102 | 72.570 | 116.969 | 1.00 | 83.10 | O |
| ATOM | 12073 | N | SER | C | 203 | 31.868 | 71.774 | 118.085 | 1.00 | 84.21 | N |
| ATOM | 12075 | CA | SER | C | 203 | 32.746 | 72.851 | 117.732 | 1.00 | 86.10 | C |
| ATOM | 12077 | CB | SER | C | 203 | 32.963 | 73.783 | 118.912 | 1.00 | 92.36 | C |
| ATOM | 12080 | OG | SER | C | 203 | 33.402 | 73.025 | 120.003 | 1.00 | 93.55 | O |
| ATOM | 12082 | C | SER | C | 203 | 34.044 | 72.186 | 117.323 | 1.00 | 83.05 | C |
| ATOM | 12083 | O | SER | C | 203 | 34.262 | 70.983 | 117.626 | 1.00 | 81.22 | O |
| ATOM | 12084 | N | PRO | C | 204 | 34.873 | 72.948 | 116.593 | 1.00 | 82.81 | N |
| ATOM | 12085 | CA | PRO | C | 204 | 36.239 | 72.533 | 116.233 | 1.00 | 79.98 | C |
| ATOM | 12087 | CB | PRO | C | 204 | 36.927 | 73.854 | 115.863 | 1.00 | 82.52 | C |
| ATOM | 12090 | CG | PRO | C | 204 | 35.815 | 74.803 | 115.476 | 1.00 | 85.07 | C |
| ATOM | 12093 | CD | PRO | C | 204 | 34.522 | 74.253 | 115.995 | 1.00 | 85.30 | C |
| ATOM | 12096 | C | PRO | C | 204 | 37.021 | 71.856 | 117.358 | 1.00 | 80.67 | C |
| ATOM | 12097 | O | PRO | C | 204 | 36.880 | 72.153 | 118.548 | 1.00 | 85.19 | O |
| ATOM | 12098 | N | VAL | C | 205 | 37.859 | 70.933 | 116.950 | 1.00 | 76.65 | N |
| ATOM | 12100 | CA | VAL | C | 205 | 38.675 | 70.224 | 117.872 | 1.00 | 77.69 | C |
| ATOM | 12102 | CB | VAL | C | 205 | 38.367 | 68.781 | 117.787 | 1.00 | 74.45 | C |
| ATOM | 12104 | CG1 | VAL | C | 205 | 39.541 | 67.929 | 118.243 | 1.00 | 74.59 | C |
| ATOM | 12108 | CG2 | VAL | C | 205 | 37.123 | 68.551 | 118.602 | 1.00 | 76.84 | C |
| ATOM | 12112 | C | VAL | C | 205 | 40.101 | 70.505 | 117.535 | 1.00 | 77.92 | C |
| ATOM | 12113 | O | VAL | C | 205 | 40.492 | 70.477 | 116.391 | 1.00 | 75.11 | O |
| ATOM | 12114 | N | THR | C | 206 | 40.873 | 70.789 | 118.566 | 1.00 | 82.28 | N |
| ATOM | 12116 | CA | THR | C | 206 | 42.253 | 71.122 | 118.415 | 1.00 | 82.99 | C |
| ATOM | 12118 | CB | THR | C | 206 | 42.454 | 72.498 | 118.958 | 1.00 | 88.12 | C |
| ATOM | 12120 | OG1 | THR | C | 206 | 41.528 | 73.357 | 118.281 | 1.00 | 89.49 | O |
| ATOM | 12122 | CG2 | THR | C | 206 | 43.880 | 73.065 | 118.602 | 1.00 | 88.94 | C |
| ATOM | 12126 | C | THR | C | 206 | 43.102 | 70.134 | 119.141 | 1.00 | 83.61 | C |
| ATOM | 12127 | O | THR | C | 206 | 42.741 | 69.648 | 120.193 | 1.00 | 86.65 | O |
| ATOM | 12128 | N | LYS | C | 207 | 44.257 | 69.863 | 118.563 | 1.00 | 82.04 | N |
| ATOM | 12130 | CA | LYS | C | 207 | 45.180 | 68.869 | 119.060 | 1.00 | 82.54 | C |
| ATOM | 12132 | CB | LYS | C | 207 | 45.015 | 67.583 | 118.263 | 1.00 | 78.30 | C |
| ATOM | 12135 | CG | LYS | C | 207 | 43.851 | 66.746 | 118.747 | 1.00 | 79.34 | C |
| ATOM | 12138 | CD | LYS | C | 207 | 44.253 | 65.776 | 119.859 | 1.00 | 83.48 | C |
| ATOM | 12141 | CE | LYS | C | 207 | 43.082 | 65.543 | 120.827 | 1.00 | 87.11 | C |
| ATOM | 12144 | NZ | LYS | C | 207 | 43.103 | 64.157 | 121.312 | 1.00 | 89.42 | N |
| ATOM | 12148 | C | LYS | C | 207 | 46.566 | 69.432 | 118.866 | 1.00 | 83.38 | C |
| ATOM | 12149 | O | LYS | C | 207 | 46.950 | 69.716 | 117.727 | 1.00 | 79.79 | O |
| ATOM | 12150 | N | SER | C | 208 | 47.307 | 69.585 | 119.974 | 1.00 | 88.00 | N |
| ATOM | 12152 | CA | SER | C | 208 | 48.616 | 70.245 | 119.940 | 1.00 | 89.86 | C |
| ATOM | 12154 | CB | SER | C | 208 | 48.480 | 71.696 | 120.405 | 1.00 | 94.01 | C |
| ATOM | 12157 | OG | SER | C | 208 | 48.739 | 71.839 | 121.786 | 1.00 | 101.12 | O |
| ATOM | 12159 | C | SER | C | 208 | 49.701 | 69.477 | 120.706 | 1.00 | 92.09 | C |
| ATOM | 12160 | O | SER | C | 208 | 49.485 | 68.338 | 121.104 | 1.00 | 91.27 | O |
| ATOM | 12161 | N | PHE | C | 209 | 50.881 | 70.092 | 120.841 | 1.00 | 94.82 | N |
| ATOM | 12163 | CA | PHE | C | 209 | 52.047 | 69.472 | 121.487 | 1.00 | 97.65 | C |
| ATOM | 12165 | CB | PHE | C | 209 | 52.583 | 68.375 | 120.587 | 1.00 | 93.33 | C |
| ATOM | 12168 | CG | PHE | C | 209 | 53.213 | 68.915 | 119.352 | 1.00 | 90.96 | C |
| ATOM | 12169 | CD1 | PHE | C | 209 | 54.542 | 69.293 | 119.354 | 1.00 | 92.49 | C |
| ATOM | 12171 | CE1 | PHE | C | 209 | 55.156 | 69.817 | 118.232 | 1.00 | 90.22 | C |
| ATOM | 12173 | CZ | PHE | C | 209 | 54.445 | 69.986 | 117.093 | 1.00 | 86.31 | C |
| ATOM | 12175 | CE2 | PHE | C | 209 | 53.081 | 69.625 | 117.063 | 1.00 | 85.26 | C |
| ATOM | 12177 | CD2 | PHE | C | 209 | 52.471 | 69.103 | 118.197 | 1.00 | 87.32 | C |
| ATOM | 12179 | C | PHE | C | 209 | 53.203 | 70.469 | 121.743 | 1.00 | 101.51 | C |
| ATOM | 12180 | O | PHE | C | 209 | 53.169 | 71.618 | 121.266 | 1.00 | 101.04 | O |
| ATOM | 12181 | N | ASN | C | 210 | 54.241 | 69.987 | 122.454 | 1.00 | 105.05 | N |
| ATOM | 12183 | CA | ASN | C | 210 | 55.489 | 70.740 | 122.698 | 1.00 | 109.04 | C |
| ATOM | 12185 | CB | ASN | C | 210 | 56.296 | 70.878 | 121.379 | 1.00 | 104.70 | C |
| ATOM | 12188 | CG | ASN | C | 210 | 57.652 | 71.550 | 121.525 | 1.00 | 107.23 | C |
| ATOM | 12189 | OD1 | ASN | C | 210 | 58.002 | 72.407 | 120.710 | 1.00 | 103.98 | O |
| ATOM | 12190 | ND2 | ASN | C | 210 | 58.447 | 71.118 | 122.493 | 1.00 | 110.21 | N |
| ATOM | 12193 | C | ASN | C | 210 | 55.024 | 72.070 | 123.298 | 1.00 | 113.97 | C |
| ATOM | 12194 | O | ASN | C | 210 | 55.282 | 73.132 | 122.742 | 1.00 | 115.08 | O |
| ATOM | 12195 | N | ARG | C | 211 | 54.328 | 71.954 | 124.444 | 1.00 | 118.00 | N |
| ATOM | 12197 | CA | ARG | C | 211 | 53.444 | 72.976 | 125.069 | 1.00 | 121.86 | C |
| ATOM | 12199 | CB | ARG | C | 211 | 54.110 | 74.377 | 125.154 | 1.00 | 126.69 | C |
| ATOM | 12208 | C | ARG | C | 211 | 52.005 | 73.001 | 124.427 | 1.00 | 117.02 | C |
| ATOM | 12209 | O | ARG | C | 211 | 51.034 | 73.584 | 124.925 | 1.00 | 119.42 | O |
| ATOM | 12210 | OXT | ARG | C | 211 | 51.618 | 72.421 | 123.385 | 1.00 | 109.85 | O |
| ATOM | 12211 | N | ALA | D | 1 | 41.982 | 30.461 | 82.277 | 1.00 | 57.00 | N |
| ATOM | 12213 | CA | ALA | D | 1 | 41.459 | 31.172 | 81.013 | 1.00 | 55.46 | C |
| ATOM | 12215 | CB | ALA | D | 1 | 39.904 | 31.400 | 81.090 | 1.00 | 56.48 | C |
| ATOM | 12219 | C | ALA | D | 1 | 42.126 | 32.481 | 80.527 | 1.00 | 50.69 | C |
| ATOM | 12220 | O | ALA | D | 1 | 43.285 | 32.669 | 80.747 | 1.00 | 52.53 | O |
| ATOM | 12223 | N | VAL | D | 2 | 41.421 | 33.378 | 79.851 | 1.00 | 47.38 | N |
| ATOM | 12225 | CA | VAL | D | 2 | 42.079 | 34.564 | 79.219 | 1.00 | 44.48 | C |
| ATOM | 12227 | CB | VAL | D | 2 | 41.096 | 35.532 | 78.483 | 1.00 | 41.76 | C |
| ATOM | 12229 | CG1 | VAL | D | 2 | 41.905 | 36.738 | 77.915 | 1.00 | 40.38 | C |
| ATOM | 12233 | CG2 | VAL | D | 2 | 40.388 | 34.848 | 77.355 | 1.00 | 39.69 | C |
| ATOM | 12237 | C | VAL | D | 2 | 42.842 | 35.498 | 80.122 | 1.00 | 44.26 | C |
| ATOM | 12238 | O | VAL | D | 2 | 42.298 | 36.058 | 81.022 | 1.00 | 44.63 | O |
| ATOM | 12239 | N | GLN | D | 3 | 44.102 | 35.758 | 79.839 | 1.00 | 45.24 | N |
| ATOM | 12241 | CA | GLN | D | 3 | 44.919 | 36.578 | 80.772 | 1.00 | 45.86 | C |
| ATOM | 12243 | CB | GLN | D | 3 | 45.720 | 35.704 | 81.745 | 1.00 | 48.64 | C |
| ATOM | 12246 | CG | GLN | D | 3 | 44.864 | 34.972 | 82.827 | 1.00 | 51.62 | C |
| ATOM | 12249 | CD | GLN | D | 3 | 45.710 | 34.463 | 84.014 | 1.00 | 59.14 | C |
| ATOM | 12250 | OE1 | GLN | D | 3 | 46.947 | 34.642 | 84.062 | 1.00 | 62.63 | O |
| ATOM | 12251 | NE2 | GLN | D | 3 | 45.053 | 33.840 | 84.973 | 1.00 | 62.87 | N |
| ATOM | 12254 | C | GLN | D | 3 | 45.815 | 37.442 | 79.938 | 1.00 | 43.72 | C |
| ATOM | 12255 | O | GLN | D | 3 | 46.250 | 36.962 | 78.925 | 1.00 | 44.39 | O |
| ATOM | 12256 | N | LEU | D | 4 | 46.002 | 38.713 | 80.310 | 1.00 | 42.40 | N |
| ATOM | 12258 | CA | LEU | D | 4 | 46.977 | 39.641 | 79.686 | 1.00 | 40.47 | C |
| ATOM | 12260 | CB | LEU | D | 4 | 46.256 | 40.846 | 79.083 | 1.00 | 37.49 | C |
| ATOM | 12263 | CG | LEU | D | 4 | 45.152 | 40.660 | 78.040 | 1.00 | 36.95 | C |
| ATOM | 12265 | CD1 | LEU | D | 4 | 45.432 | 41.577 | 76.858 | 1.00 | 36.62 | C |
| ATOM | 12269 | CD2 | LEU | D | 4 | 45.043 | 39.356 | 77.504 | 1.00 | 39.31 | C |
| ATOM | 12273 | C | LEU | D | 4 | 47.969 | 40.192 | 80.723 | 1.00 | 41.49 | C |
| ATOM | 12274 | O | LEU | D | 4 | 47.599 | 40.842 | 81.665 | 1.00 | 41.51 | O |
| ATOM | 12275 | N | LYS | D | 5 | 49.250 | 39.970 | 80.549 | 1.00 | 43.23 | N |
| ATOM | 12277 | CA | LYS | D | 5 | 50.217 | 40.446 | 81.543 | 1.00 | 45.14 | C |
| ATOM | 12279 | CB | LYS | D | 5 | 50.892 | 39.284 | 82.282 | 1.00 | 48.88 | C |
| ATOM | 12282 | CG | LYS | D | 5 | 49.903 | 38.536 | 83.266 | 1.00 | 54.47 | C |
| ATOM | 12285 | CD | LYS | D | 5 | 50.560 | 37.297 | 83.990 | 1.00 | 60.79 | C |
| ATOM | 12288 | CE | LYS | D | 5 | 49.635 | 36.013 | 83.904 | 1.00 | 63.31 | C |
| ATOM | 12291 | NZ | LYS | D | 5 | 50.408 | 34.721 | 84.358 | 1.00 | 69.55 | N |
| ATOM | 12295 | C | LYS | D | 5 | 51.231 | 41.305 | 80.900 | 1.00 | 43.29 | C |
| ATOM | 12296 | O | LYS | D | 5 | 51.789 | 40.931 | 79.897 | 1.00 | 43.73 | O |
| ATOM | 12297 | N | GLN | D | 6 | 51.474 | 42.461 | 81.495 | 1.00 | 42.41 | N |
| ATOM | 12299 | CA | GLN | D | 6 | 52.383 | 43.434 | 80.924 | 1.00 | 41.52 | C |
| ATOM | 12301 | CB | GLN | D | 6 | 51.796 | 44.835 | 81.003 | 1.00 | 40.15 | C |
| ATOM | 12304 | CG | GLN | D | 6 | 50.406 | 44.997 | 80.357 | 1.00 | 39.53 | C |
| ATOM | 12307 | CD | GLN | D | 6 | 49.771 | 46.341 | 80.616 | 1.00 | 38.13 | C |
| ATOM | 12308 | OE1 | GLN | D | 6 | 50.442 | 47.334 | 80.783 | 1.00 | 39.92 | O |
| ATOM | 12309 | NE2 | GLN | D | 6 | 48.468 | 46.370 | 80.597 | 1.00 | 40.10 | N |
| ATOM | 12312 | C | GLN | D | 6 | 53.668 | 43.410 | 81.676 | 1.00 | 43.59 | C |
| ATOM | 12313 | O | GLN | D | 6 | 53.675 | 43.182 | 82.887 | 1.00 | 43.49 | O |
| ATOM | 12314 | N | SER | D | 7 | 54.749 | 43.568 | 80.920 | 1.00 | 44.87 | N |
| ATOM | 12316 | CA | SER | D | 7 | 56.016 | 44.080 | 81.435 | 1.00 | 48.01 | C |
| ATOM | 12318 | CB | SER | D | 7 | 57.107 | 42.989 | 81.406 | 1.00 | 51.52 | C |
| ATOM | 12321 | OG | SER | D | 7 | 57.386 | 42.519 | 80.095 | 1.00 | 51.53 | O |
| ATOM | 12323 | C | SER | D | 7 | 56.521 | 45.303 | 80.675 | 1.00 | 47.70 | C |
| ATOM | 12324 | O | SER | D | 7 | 56.324 | 45.395 | 79.469 | 1.00 | 46.37 | O |
| ATOM | 12325 | N | GLY | D | 8 | 57.216 | 46.210 | 81.364 | 1.00 | 50.18 | N |
| ATOM | 12327 | CA | GLY | D | 8 | 57.911 | 47.315 | 80.713 | 1.00 | 50.86 | C |
| ATOM | 12330 | C | GLY | D | 8 | 58.393 | 48.422 | 81.619 | 1.00 | 52.58 | C |
| ATOM | 12331 | O | GLY | D | 8 | 57.780 | 48.763 | 82.605 | 1.00 | 51.07 | O |
| ATOM | 12332 | N | PRO | D | 9 | 59.516 | 49.021 | 81.266 | 1.00 | 56.53 | N |
| ATOM | 12333 | CA | PRO | D | 9 | 60.030 | 50.159 | 82.043 | 1.00 | 58.30 | C |
| ATOM | 12335 | CB | PRO | D | 9 | 61.288 | 50.586 | 81.284 | 1.00 | 61.09 | C |
| ATOM | 12338 | CG | PRO | D | 9 | 61.824 | 49.267 | 80.591 | 1.00 | 62.53 | C |
| ATOM | 12341 | CD | PRO | D | 9 | 60.435 | 48.657 | 80.162 | 1.00 | 58.96 | C |
| ATOM | 12344 | C | PRO | D | 9 | 59.005 | 51.228 | 82.035 | 1.00 | 56.45 | C |
| ATOM | 12345 | O | PRO | D | 9 | 58.635 | 51.619 | 80.919 | 1.00 | 55.51 | O |
| ATOM | 12346 | N | GLY | D | 10 | 58.485 | 51.541 | 83.237 | 1.00 | 56.24 | N |
| ATOM | 12348 | CA | GLY | D | 10 | 57.666 | 52.714 | 83.525 | 1.00 | 54.81 | C |
| ATOM | 12351 | C | GLY | D | 10 | 58.271 | 54.154 | 83.662 | 1.00 | 55.43 | C |
| ATOM | 12352 | O | GLY | D | 10 | 57.660 | 55.095 | 83.144 | 1.00 | 53.90 | O |
| ATOM | 12353 | N | LEU | D | 11 | 59.401 | 54.365 | 84.340 | 1.00 | 56.80 | N |
| ATOM | 12355 | CA | LEU | D | 11 | 60.044 | 55.699 | 84.289 | 1.00 | 57.96 | C |
| ATOM | 12357 | CB | LEU | D | 11 | 61.148 | 55.834 | 85.329 | 1.00 | 61.18 | C |
| ATOM | 12360 | CG | LEU | D | 11 | 61.792 | 57.218 | 85.406 | 1.00 | 62.40 | C |
| ATOM | 12362 | CD1 | LEU | D | 11 | 60.836 | 58.390 | 85.187 | 1.00 | 61.71 | C |
| ATOM | 12366 | CD2 | LEU | D | 11 | 62.429 | 57.397 | 86.726 | 1.00 | 63.46 | C |
| ATOM | 12370 | C | LEU | D | 11 | 60.627 | 55.983 | 82.904 | 1.00 | 57.05 | C |
| ATOM | 12371 | O | LEU | D | 11 | 61.347 | 55.208 | 82.383 | 1.00 | 58.70 | O |
| ATOM | 12372 | N | VAL | D | 12 | 60.292 | 57.085 | 82.291 | 1.00 | 55.69 | N |
| ATOM | 12374 | CA | VAL | D | 12 | 60.629 | 57.284 | 80.914 | 1.00 | 55.00 | C |
| ATOM | 12376 | CB | VAL | D | 12 | 59.409 | 57.062 | 80.020 | 1.00 | 52.03 | C |
| ATOM | 12378 | CG1 | VAL | D | 12 | 59.667 | 57.583 | 78.591 | 1.00 | 54.04 | C |
| ATOM | 12382 | CG2 | VAL | D | 12 | 59.123 | 55.658 | 79.921 | 1.00 | 50.83 | C |
| ATOM | 12386 | C | VAL | D | 12 | 61.019 | 58.701 | 80.732 | 1.00 | 56.49 | C |
| ATOM | 12387 | O | VAL | D | 12 | 60.264 | 59.589 | 81.020 | 1.00 | 55.45 | O |
| ATOM | 12388 | N | GLN | D | 13 | 62.182 | 58.916 | 80.163 | 1.00 | 59.59 | N |
| ATOM | 12390 | CA | GLN | D | 13 | 62.707 | 60.278 | 80.045 | 1.00 | 62.52 | C |
| ATOM | 12392 | CB | GLN | D | 13 | 64.212 | 60.305 | 79.899 | 1.00 | 65.71 | C |
| ATOM | 12395 | CG | GLN | D | 13 | 64.776 | 60.399 | 81.258 | 1.00 | 69.08 | C |
| ATOM | 12398 | CD | GLN | D | 13 | 66.219 | 60.422 | 81.271 | 1.00 | 77.09 | C |
| ATOM | 12399 | OE1 | GLN | D | 13 | 66.874 | 60.460 | 80.225 | 1.00 | 81.75 | O |
| ATOM | 12400 | NE2 | GLN | D | 13 | 66.770 | 60.414 | 82.464 | 1.00 | 82.68 | N |
| ATOM | 12403 | C | GLN | D | 13 | 62.064 | 60.980 | 78.931 | 1.00 | 61.31 | C |
| ATOM | 12404 | O | GLN | D | 13 | 61.775 | 60.374 | 77.918 | 1.00 | 60.31 | O |
| ATOM | 12405 | N | PRO | D | 14 | 61.769 | 62.245 | 79.137 | 1.00 | 62.39 | N |
| ATOM | 12406 | CA | PRO | D | 14 | 61.018 | 62.986 | 78.155 | 1.00 | 61.94 | C |
| ATOM | 12408 | CB | PRO | D | 14 | 60.741 | 64.296 | 78.849 | 1.00 | 63.94 | C |
| ATOM | 12411 | CG | PRO | D | 14 | 61.829 | 64.410 | 79.730 | 1.00 | 66.71 | C |
| ATOM | 12414 | CD | PRO | D | 14 | 62.070 | 63.082 | 80.291 | 1.00 | 64.87 | C |
| ATOM | 12417 | C | PRO | D | 14 | 61.835 | 63.107 | 76.888 | 1.00 | 63.56 | C |
| ATOM | 12418 | O | PRO | D | 14 | 63.036 | 63.175 | 76.841 | 1.00 | 63.90 | O |
| ATOM | 12419 | N | SER | D | 15 | 61.050 | 62.965 | 75.841 | 1.00 | 62.98 | N |
| ATOM | 12421 | CA | SER | D | 15 | 61.451 | 62.904 | 74.452 | 1.00 | 64.41 | C |
| ATOM | 12423 | CB | SER | D | 15 | 62.324 | 64.072 | 74.064 | 1.00 | 67.55 | C |
| ATOM | 12426 | OG | SER | D | 15 | 63.444 | 63.478 | 73.590 | 1.00 | 66.46 | O |
| ATOM | 12428 | C | SER | D | 15 | 62.057 | 61.533 | 74.072 | 1.00 | 64.44 | C |
| ATOM | 12429 | O | SER | D | 15 | 62.277 | 61.275 | 72.902 | 1.00 | 65.45 | O |
| ATOM | 12430 | N | GLN | D | 16 | 62.212 | 60.620 | 75.032 | 1.00 | 64.20 | N |
| ATOM | 12432 | CA | GLN | D | 16 | 62.561 | 59.213 | 74.717 | 1.00 | 63.39 | C |
| ATOM | 12434 | CB | GLN | D | 16 | 63.626 | 58.733 | 75.704 | 1.00 | 65.81 | C |
| ATOM | 12437 | CG | GLN | D | 16 | 64.952 | 58.300 | 75.040 | 1.00 | 71.75 | C |
| ATOM | 12440 | CD | GLN | D | 16 | 65.920 | 58.044 | 76.159 | 1.00 | 80.91 | C |
| ATOM | 12441 | OE1 | GLN | D | 16 | 65.494 | 58.154 | 77.339 | 1.00 | 85.18 | O |
| ATOM | 12442 | NE2 | GLN | D | 16 | 67.191 | 57.708 | 75.856 | 1.00 | 83.89 | N |
| ATOM | 12445 | C | GLN | D | 16 | 61.346 | 58.241 | 74.614 | 1.00 | 57.84 | C |
| ATOM | 12446 | O | GLN | D | 16 | 60.221 | 58.667 | 74.789 | 1.00 | 56.16 | O |
| ATOM | 12447 | N | SER | D | 17 | 61.557 | 56.958 | 74.323 | 1.00 | 56.20 | N |
| ATOM | 12449 | CA | SER | D | 17 | 60.435 | 56.035 | 74.025 | 1.00 | 53.11 | C |
| ATOM | 12451 | CB | SER | D | 17 | 60.826 | 55.185 | 72.839 | 1.00 | 53.56 | C |
| ATOM | 12454 | OG | SER | D | 17 | 61.347 | 53.954 | 73.336 | 1.00 | 55.78 | O |
| ATOM | 12456 | C | SER | D | 17 | 59.885 | 55.054 | 75.152 | 1.00 | 50.62 | C |
| ATOM | 12457 | O | SER | D | 17 | 60.571 | 54.565 | 75.997 | 1.00 | 53.47 | O |
| ATOM | 12458 | N | LEU | D | 18 | 58.624 | 54.752 | 75.101 | 1.00 | 46.73 | N |
| ATOM | 12460 | CA | LEU | D | 18 | 57.978 | 53.917 | 76.046 | 1.00 | 44.77 | C |
| ATOM | 12462 | CB | LEU | D | 18 | 56.567 | 54.511 | 76.298 | 1.00 | 42.30 | C |
| ATOM | 12465 | CG | LEU | D | 18 | 55.563 | 53.620 | 76.986 | 1.00 | 40.07 | C |
| ATOM | 12467 | CD1 | LEU | D | 18 | 56.090 | 53.066 | 78.283 | 1.00 | 41.41 | C |
| ATOM | 12471 | CD2 | LEU | D | 18 | 54.332 | 54.437 | 77.255 | 1.00 | 41.25 | C |
| ATOM | 12475 | C | LEU | D | 18 | 57.806 | 52.599 | 75.327 | 1.00 | 43.93 | C |
| ATOM | 12476 | O | LEU | D | 18 | 57.148 | 52.565 | 74.292 | 1.00 | 42.76 | O |
| ATOM | 12477 | N | SER | D | 19 | 58.323 | 51.506 | 75.879 | 1.00 | 44.90 | N |
| ATOM | 12479 | CA | SER | D | 19 | 58.085 | 50.145 | 75.341 | 1.00 | 43.56 | C |
| ATOM | 12481 | CB | SER | D | 19 | 59.401 | 49.524 | 74.816 | 1.00 | 46.48 | C |
| ATOM | 12484 | OG | SER | D | 19 | 59.933 | 50.140 | 73.634 | 1.00 | 46.71 | O |
| ATOM | 12486 | C | SER | D | 19 | 57.448 | 49.240 | 76.426 | 1.00 | 42.19 | C |
| ATOM | 12487 | O | SER | D | 19 | 57.952 | 49.152 | 77.562 | 1.00 | 44.56 | O |
| ATOM | 12488 | N | ILE | D | 20 | 56.343 | 48.596 | 76.071 | 1.00 | 39.20 | N |
| ATOM | 12490 | CA | ILE | D | 20 | 55.610 | 47.698 | 76.923 | 1.00 | 38.53 | C |
| ATOM | 12492 | CB | ILE | D | 20 | 54.240 | 48.329 | 77.293 | 1.00 | 37.18 | C |
| ATOM | 12494 | CG1 | ILE | D | 20 | 54.377 | 49.567 | 78.173 | 1.00 | 37.34 | C |
| ATOM | 12497 | CD1 | ILE | D | 20 | 53.211 | 50.449 | 78.017 | 1.00 | 39.17 | C |
| ATOM | 12501 | CG2 | ILE | D | 20 | 53.280 | 47.309 | 78.017 | 1.00 | 37.55 | C |
| ATOM | 12505 | C | ILE | D | 20 | 55.324 | 46.437 | 76.164 | 1.00 | 38.06 | C |
| ATOM | 12506 | O | ILE | D | 20 | 54.932 | 46.471 | 75.000 | 1.00 | 36.64 | O |
| ATOM | 12507 | N | THR | D | 21 | 55.441 | 45.303 | 76.835 | 1.00 | 40.37 | N |
| ATOM | 12509 | CA | THR | D | 21 | 55.088 | 44.001 | 76.249 | 1.00 | 41.04 | C |
| ATOM | 12511 | CB | THR | D | 21 | 56.261 | 43.068 | 76.450 | 1.00 | 43.74 | C |
| ATOM | 12513 | OG1 | THR | D | 21 | 57.415 | 43.589 | 75.748 | 1.00 | 46.48 | O |
| ATOM | 12515 | CG2 | THR | D | 21 | 55.958 | 41.774 | 75.812 | 1.00 | 45.01 | C |
| ATOM | 12519 | C | THR | D | 21 | 53.813 | 43.430 | 76.933 | 1.00 | 40.58 | C |
| ATOM | 12520 | O | THR | D | 21 | 53.626 | 43.567 | 78.162 | 1.00 | 41.40 | O |
| ATOM | 12521 | N | CYS | D | 22 | 52.939 | 42.816 | 76.145 | 1.00 | 39.61 | N |
| ATOM | 12523 | CA | CYS | D | 22 | 51.711 | 42.179 | 76.631 | 1.00 | 39.65 | C |
| ATOM | 12525 | CB | CYS | D | 22 | 50.514 | 42.703 | 75.891 | 1.00 | 36.18 | C |
| ATOM | 12528 | SG | CYS | D | 22 | 48.888 | 42.439 | 76.557 | 1.00 | 40.37 | S |
| ATOM | 12529 | C | CYS | D | 22 | 51.943 | 40.719 | 76.274 | 1.00 | 41.94 | C |
| ATOM | 12530 | O | CYS | D | 22 | 51.961 | 40.359 | 75.089 | 1.00 | 42.48 | O |
| ATOM | 12531 | N | THR | D | 23 | 52.199 | 39.891 | 77.285 | 1.00 | 43.68 | N |
| ATOM | 12533 | CA | THR | D | 23 | 52.093 | 38.468 | 77.096 | 1.00 | 45.36 | C |
| ATOM | 12535 | CB | THR | D | 23 | 53.107 | 37.752 | 77.925 | 1.00 | 48.80 | C |
| ATOM | 12537 | OG1 | THR | D | 23 | 54.348 | 38.483 | 77.823 | 1.00 | 53.25 | O |
| ATOM | 12539 | CG2 | THR | D | 23 | 53.391 | 36.405 | 77.328 | 1.00 | 48.29 | C |
| ATOM | 12543 | C | THR | D | 23 | 50.749 | 37.970 | 77.417 | 1.00 | 44.01 | C |
| ATOM | 12544 | O | THR | D | 23 | 50.309 | 38.115 | 78.469 | 1.00 | 45.58 | O |
| ATOM | 12545 | N | VAL | D | 24 | 50.189 | 37.240 | 76.505 | 1.00 | 44.18 | N |
| ATOM | 12547 | CA | VAL | D | 24 | 48.801 | 36.874 | 76.441 | 1.00 | 43.47 | C |
| ATOM | 12549 | CB | VAL | D | 24 | 48.356 | 37.223 | 74.983 | 1.00 | 41.96 | C |
| ATOM | 12551 | CG1 | VAL | D | 24 | 47.387 | 36.320 | 74.500 | 1.00 | 46.10 | C |
| ATOM | 12555 | CG2 | VAL | D | 24 | 47.785 | 38.612 | 74.857 | 1.00 | 38.96 | C |
| ATOM | 12559 | C | VAL | D | 24 | 48.716 | 35.342 | 76.633 | 1.00 | 45.93 | C |
| ATOM | 12560 | O | VAL | D | 24 | 49.555 | 34.625 | 76.140 | 1.00 | 47.90 | O |
| ATOM | 12561 | N | SER | D | 25 | 47.696 | 34.840 | 77.320 | 1.00 | 46.33 | N |
| ATOM | 12563 | CA | SER | D | 25 | 47.435 | 33.388 | 77.384 | 1.00 | 48.67 | C |
| ATOM | 12565 | CB | SER | D | 25 | 48.043 | 32.805 | 78.617 | 1.00 | 51.18 | C |
| ATOM | 12568 | OG | SER | D | 25 | 47.357 | 33.326 | 79.724 | 1.00 | 50.94 | O |
| ATOM | 12570 | C | SER | D | 25 | 45.932 | 33.060 | 77.394 | 1.00 | 47.97 | C |
| ATOM | 12571 | O | SER | D | 25 | 45.123 | 33.821 | 77.850 | 1.00 | 46.72 | O |
| ATOM | 12572 | N | GLY | D | 26 | 45.566 | 31.903 | 76.896 | 1.00 | 49.61 | N |
| ATOM | 12574 | CA | GLY | D | 26 | 44.195 | 31.497 | 76.922 | 1.00 | 49.63 | C |
| ATOM | 12577 | C | GLY | D | 26 | 43.485 | 31.917 | 75.682 | 1.00 | 47.39 | C |
| ATOM | 12578 | O | GLY | D | 26 | 42.289 | 31.683 | 75.579 | 1.00 | 48.65 | O |
| ATOM | 12579 | N | PHE | D | 27 | 44.175 | 32.578 | 74.766 | 1.00 | 44.85 | N |
| ATOM | 12581 | CA | PHE | D | 27 | 43.678 | 32.695 | 73.407 | 1.00 | 42.48 | C |
| ATOM | 12583 | CB | PHE | D | 27 | 42.750 | 33.823 | 73.310 | 1.00 | 38.91 | C |
| ATOM | 12586 | CG | PHE | D | 27 | 43.398 | 35.137 | 73.470 | 1.00 | 37.97 | C |
| ATOM | 12587 | CD1 | PHE | D | 27 | 43.723 | 35.946 | 72.327 | 1.00 | 36.52 | C |
| ATOM | 12589 | CE1 | PHE | D | 27 | 44.286 | 37.244 | 72.465 | 1.00 | 32.40 | C |
| ATOM | 12591 | CZ | PHE | D | 27 | 44.529 | 37.730 | 73.713 | 1.00 | 33.39 | C |
| ATOM | 12593 | CE2 | PHE | D | 27 | 44.200 | 36.928 | 74.876 | 1.00 | 36.37 | C |
| ATOM | 12595 | CD2 | PHE | D | 27 | 43.631 | 35.636 | 74.733 | 1.00 | 37.60 | C |
| ATOM | 12597 | C | PHE | D | 27 | 44.849 | 32.968 | 72.521 | 1.00 | 42.29 | C |
| ATOM | 12598 | O | PHE | D | 27 | 45.885 | 33.253 | 73.043 | 1.00 | 43.18 | O |
| ATOM | 12599 | N | SER | D | 28 | 44.701 | 32.894 | 71.199 | 1.00 | 41.48 | N |
| ATOM | 12601 | CA | SER | D | 28 | 45.787 | 33.229 | 70.284 | 1.00 | 41.06 | C |
| ATOM | 12603 | CB | SER | D | 28 | 46.052 | 32.076 | 69.303 | 1.00 | 43.62 | C |
| ATOM | 12606 | OG | SER | D | 28 | 45.379 | 32.165 | 68.103 | 1.00 | 42.29 | O |
| ATOM | 12608 | C | SER | D | 28 | 45.579 | 34.557 | 69.592 | 1.00 | 38.22 | C |
| ATOM | 12609 | O | SER | D | 28 | 44.467 | 34.940 | 69.333 | 1.00 | 36.54 | O |
| ATOM | 12610 | N | LEU | D | 29 | 46.675 | 35.275 | 69.369 | 1.00 | 38.34 | N |
| ATOM | 12612 | CA | LEU | D | 29 | 46.673 | 36.543 | 68.643 | 1.00 | 36.43 | C |
| ATOM | 12614 | CB | LEU | D | 29 | 48.007 | 37.269 | 68.769 | 1.00 | 35.93 | C |
| ATOM | 12617 | CG | LEU | D | 29 | 48.340 | 37.596 | 70.229 | 1.00 | 37.34 | C |
| ATOM | 12619 | CD1 | LEU | D | 29 | 49.853 | 37.698 | 70.436 | 1.00 | 42.57 | C |
| ATOM | 12623 | CD2 | LEU | D | 29 | 47.767 | 38.823 | 70.769 | 1.00 | 35.50 | C |
| ATOM | 12627 | C | LEU | D | 29 | 46.360 | 36.329 | 67.182 | 1.00 | 36.97 | C |
| ATOM | 12628 | O | LEU | D | 29 | 46.087 | 37.243 | 66.425 | 1.00 | 36.62 | O |
| ATOM | 12629 | N | THR | D | 30 | 46.351 | 35.089 | 66.796 | 1.00 | 39.55 | N |
| ATOM | 12631 | CA | THR | D | 30 | 45.918 | 34.679 | 65.473 | 1.00 | 39.97 | C |
| ATOM | 12633 | CB | THR | D | 30 | 46.294 | 33.210 | 65.329 | 1.00 | 43.13 | C |
| ATOM | 12635 | OG1 | THR | D | 30 | 47.406 | 33.151 | 64.447 | 1.00 | 45.16 | O |
| ATOM | 12637 | CG2 | THR | D | 30 | 45.192 | 32.267 | 64.762 | 1.00 | 43.76 | C |
| ATOM | 12641 | C | THR | D | 30 | 44.454 | 34.936 | 65.221 | 1.00 | 38.74 | C |
| ATOM | 12642 | O | THR | D | 30 | 44.112 | 35.136 | 64.063 | 1.00 | 39.61 | O |
| ATOM | 12643 | N | ASN | D | 31 | 43.611 | 34.983 | 66.267 | 1.00 | 37.45 | N |
| ATOM | 12645 | CA | ASN | D | 31 | 42.163 | 35.188 | 66.094 | 1.00 | 36.67 | C |
| ATOM | 12647 | CB | ASN | D | 31 | 41.384 | 34.034 | 66.706 | 1.00 | 38.21 | C |
| ATOM | 12650 | CG | ASN | D | 31 | 41.839 | 32.691 | 66.202 | 1.00 | 40.54 | C |
| ATOM | 12651 | OD1 | ASN | D | 31 | 42.034 | 32.490 | 64.993 | 1.00 | 41.36 | O |
| ATOM | 12652 | ND2 | ASN | D | 31 | 42.022 | 31.763 | 67.125 | 1.00 | 40.98 | N |
| ATOM | 12655 | C | ASN | D | 31 | 41.563 | 36.434 | 66.711 | 1.00 | 34.82 | C |
| ATOM | 12656 | O | ASN | D | 31 | 40.386 | 36.809 | 66.403 | 1.00 | 34.97 | O |
| ATOM | 12657 | N | TYR | D | 32 | 42.328 | 37.047 | 67.606 | 1.00 | 33.79 | N |
| ATOM | 12659 | CA | TYR | D | 32 | 41.897 | 38.238 | 68.386 | 1.00 | 31.32 | C |
| ATOM | 12661 | CB | TYR | D | 32 | 41.680 | 37.931 | 69.881 | 1.00 | 30.94 | C |
| ATOM | 12664 | CG | TYR | D | 32 | 40.436 | 37.197 | 70.158 | 1.00 | 31.75 | C |
| ATOM | 12665 | CD1 | TYR | D | 32 | 40.437 | 35.834 | 70.261 | 1.00 | 35.96 | C |
| ATOM | 12667 | CE1 | TYR | D | 32 | 39.263 | 35.109 | 70.544 | 1.00 | 37.48 | C |
| ATOM | 12669 | CZ | TYR | D | 32 | 38.076 | 35.762 | 70.634 | 1.00 | 38.25 | C |
| ATOM | 12670 | OH | TYR | D | 32 | 36.944 | 35.006 | 70.814 | 1.00 | 42.78 | O |
| ATOM | 12672 | CE2 | TYR | D | 32 | 38.032 | 37.152 | 70.457 | 1.00 | 36.56 | C |
| ATOM | 12674 | CD2 | TYR | D | 32 | 39.225 | 37.857 | 70.249 | 1.00 | 33.48 | C |
| ATOM | 12676 | C | TYR | D | 32 | 42.934 | 39.293 | 68.222 | 1.00 | 29.57 | C |
| ATOM | 12677 | O | TYR | D | 32 | 44.098 | 38.983 | 68.095 | 1.00 | 32.81 | O |
| ATOM | 12678 | N | GLY | D | 33 | 42.505 | 40.521 | 68.175 | 1.00 | 27.55 | N |
| ATOM | 12680 | CA | GLY | D | 33 | 43.339 | 41.698 | 68.179 | 1.00 | 27.54 | C |
| ATOM | 12683 | C | GLY | D | 33 | 43.622 | 42.083 | 69.643 | 1.00 | 28.10 | C |
| ATOM | 12684 | O | GLY | D | 33 | 42.841 | 41.699 | 70.478 | 1.00 | 28.48 | O |
| ATOM | 12685 | N | VAL | D | 34 | 44.747 | 42.753 | 69.946 | 1.00 | 28.42 | N |
| ATOM | 12687 | CA | VAL | D | 34 | 44.978 | 43.335 | 71.246 | 1.00 | 28.37 | C |
| ATOM | 12689 | CB | VAL | D | 34 | 46.329 | 42.870 | 71.774 | 1.00 | 29.29 | C |
| ATOM | 12691 | CG1 | VAL | D | 34 | 46.618 | 43.435 | 73.133 | 1.00 | 29.09 | C |
| ATOM | 12695 | CG2 | VAL | D | 34 | 46.332 | 41.434 | 71.903 | 1.00 | 31.00 | C |
| ATOM | 12699 | C | VAL | D | 34 | 44.958 | 44.866 | 71.101 | 1.00 | 28.37 | C |
| ATOM | 12700 | O | VAL | D | 34 | 45.624 | 45.432 | 70.231 | 1.00 | 28.84 | O |
| ATOM | 12701 | N | HIS | D | 35 | 44.195 | 45.528 | 71.940 | 1.00 | 28.35 | N |
| ATOM | 12703 | CA | HIS | D | 35 | 44.057 | 46.977 | 71.908 | 1.00 | 29.07 | C |
| ATOM | 12705 | CB | HIS | D | 35 | 42.582 | 47.405 | 72.135 | 1.00 | 28.80 | C |
| ATOM | 12708 | CG | HIS | D | 35 | 41.629 | 46.857 | 71.124 | 1.00 | 28.13 | C |
| ATOM | 12709 | ND1 | HIS | D | 35 | 41.245 | 47.557 | 70.018 | 1.00 | 28.65 | N |
| ATOM | 12711 | CE1 | HIS | D | 35 | 40.387 | 46.852 | 69.309 | 1.00 | 28.35 | C |
| ATOM | 12713 | NE2 | HIS | D | 35 | 40.220 | 45.689 | 69.902 | 1.00 | 29.20 | N |
| ATOM | 12715 | CD2 | HIS | D | 35 | 40.962 | 45.687 | 71.062 | 1.00 | 30.52 | C |
| ATOM | 12717 | C | HIS | D | 35 | 44.914 | 47.588 | 72.996 | 1.00 | 30.05 | C |
| ATOM | 12718 | O | HIS | D | 35 | 45.252 | 46.902 | 73.960 | 1.00 | 31.00 | O |
| ATOM | 12719 | N | TRP | D | 36 | 45.191 | 48.891 | 72.888 | 1.00 | 30.37 | N |
| ATOM | 12721 | CA | TRP | D | 36 | 45.968 | 49.637 | 73.896 | 1.00 | 30.27 | C |
| ATOM | 12723 | CB | TRP | D | 36 | 47.309 | 50.002 | 73.310 | 1.00 | 30.97 | C |
| ATOM | 12726 | CG | TRP | D | 36 | 48.150 | 48.741 | 73.152 | 1.00 | 33.79 | C |
| ATOM | 12727 | CD1 | TRP | D | 36 | 48.170 | 47.889 | 72.086 | 1.00 | 35.19 | C |
| ATOM | 12729 | NE1 | TRP | D | 36 | 49.026 | 46.835 | 72.340 | 1.00 | 35.82 | N |
| ATOM | 12731 | CE2 | TRP | D | 36 | 49.551 | 46.981 | 73.589 | 1.00 | 34.05 | C |
| ATOM | 12732 | CD2 | TRP | D | 36 | 49.040 | 48.169 | 74.124 | 1.00 | 33.66 | C |
| ATOM | 12733 | CE3 | TRP | D | 36 | 49.467 | 48.562 | 75.374 | 1.00 | 33.15 | C |
| ATOM | 12735 | CZ3 | TRP | D | 36 | 50.354 | 47.796 | 76.024 | 1.00 | 37.51 | C |
| ATOM | 12737 | CH2 | TRP | D | 36 | 50.881 | 46.636 | 75.451 | 1.00 | 38.07 | C |
| ATOM | 12739 | CZ2 | TRP | D | 36 | 50.470 | 46.216 | 74.235 | 1.00 | 37.73 | C |
| ATOM | 12741 | C | TRP | D | 36 | 45.246 | 50.892 | 74.309 | 1.00 | 30.62 | C |
| ATOM | 12742 | O | TRP | D | 36 | 44.780 | 51.687 | 73.485 | 1.00 | 31.69 | O |
| ATOM | 12743 | N | VAL | D | 37 | 45.089 | 51.077 | 75.591 | 1.00 | 30.38 | N |
| ATOM | 12745 | CA | VAL | D | 37 | 44.250 | 52.184 | 76.057 | 1.00 | 30.87 | C |
| ATOM | 12747 | CB | VAL | D | 37 | 42.965 | 51.664 | 76.601 | 1.00 | 30.04 | C |
| ATOM | 12749 | CG1 | VAL | D | 37 | 42.249 | 52.681 | 77.266 | 1.00 | 29.29 | C |
| ATOM | 12753 | CG2 | VAL | D | 37 | 42.196 | 51.029 | 75.509 | 1.00 | 29.91 | C |
| ATOM | 12757 | C | VAL | D | 37 | 45.001 | 52.685 | 77.207 | 1.00 | 32.03 | C |
| ATOM | 12758 | O | VAL | D | 37 | 45.581 | 51.865 | 77.898 | 1.00 | 33.02 | O |
| ATOM | 12759 | N | ARG | D | 38 | 44.997 | 53.987 | 77.438 | 1.00 | 32.94 | N |
| ATOM | 12761 | CA | ARG | D | 38 | 45.661 | 54.526 | 78.606 | 1.00 | 34.28 | C |
| ATOM | 12763 | CB | ARG | D | 38 | 46.875 | 55.319 | 78.163 | 1.00 | 35.74 | C |
| ATOM | 12766 | CG | ARG | D | 38 | 46.557 | 56.678 | 77.574 | 1.00 | 36.42 | C |
| ATOM | 12769 | CD | ARG | D | 38 | 47.776 | 57.442 | 77.251 | 1.00 | 36.01 | C |
| ATOM | 12772 | NE | ARG | D | 38 | 47.459 | 58.672 | 76.558 | 1.00 | 38.09 | N |
| ATOM | 12774 | CZ | ARG | D | 38 | 48.345 | 59.511 | 76.121 | 1.00 | 36.79 | C |
| ATOM | 12775 | NH1 | ARG | D | 38 | 49.623 | 59.276 | 76.303 | 1.00 | 39.89 | N |
| ATOM | 12778 | NH2 | ARG | D | 38 | 47.960 | 60.562 | 75.501 | 1.00 | 38.50 | N |
| ATOM | 12781 | C | ARG | D | 38 | 44.763 | 55.369 | 79.527 | 1.00 | 34.80 | C |
| ATOM | 12782 | O | ARG | D | 38 | 43.781 | 55.900 | 79.125 | 1.00 | 33.76 | O |
| ATOM | 12783 | N | GLN | D | 39 | 45.159 | 55.528 | 80.755 | 1.00 | 35.60 | N |
| ATOM | 12785 | CA | GLN | D | 39 | 44.335 | 56.277 | 81.633 | 1.00 | 38.73 | C |
| ATOM | 12787 | CB | GLN | D | 39 | 43.724 | 55.284 | 82.598 | 1.00 | 39.56 | C |
| ATOM | 12790 | CG | GLN | D | 39 | 42.743 | 55.887 | 83.573 | 1.00 | 41.47 | C |
| ATOM | 12793 | CD | GLN | D | 39 | 42.015 | 54.861 | 84.346 | 1.00 | 40.76 | C |
| ATOM | 12794 | OE1 | GLN | D | 39 | 42.588 | 53.843 | 84.731 | 1.00 | 40.61 | O |
| ATOM | 12795 | NE2 | GLN | D | 39 | 40.736 | 55.120 | 84.599 | 1.00 | 45.06 | N |
| ATOM | 12798 | C | GLN | D | 39 | 45.148 | 57.376 | 82.371 | 1.00 | 41.44 | C |
| ATOM | 12799 | O | GLN | D | 39 | 46.199 | 57.117 | 82.951 | 1.00 | 41.23 | O |
| ATOM | 12800 | N | SER | D | 40 | 44.701 | 58.612 | 82.294 | 1.00 | 43.95 | N |
| ATOM | 12802 | CA | SER | D | 40 | 45.297 | 59.665 | 83.117 | 1.00 | 48.33 | C |
| ATOM | 12804 | CB | SER | D | 40 | 45.996 | 60.801 | 82.338 | 1.00 | 49.57 | C |
| ATOM | 12807 | OG | SER | D | 40 | 45.266 | 61.127 | 81.159 | 1.00 | 48.49 | O |
| ATOM | 12809 | C | SER | D | 40 | 44.162 | 60.222 | 83.917 | 1.00 | 51.39 | C |
| ATOM | 12810 | O | SER | D | 40 | 43.005 | 60.374 | 83.420 | 1.00 | 53.29 | O |
| ATOM | 12811 | N | PRO | D | 41 | 44.470 | 60.547 | 85.153 | 1.00 | 53.68 | N |
| ATOM | 12812 | CA | PRO | D | 41 | 43.423 | 60.996 | 86.069 | 1.00 | 57.08 | C |
| ATOM | 12814 | CB | PRO | D | 41 | 44.192 | 61.252 | 87.364 | 1.00 | 58.57 | C |
| ATOM | 12817 | CG | PRO | D | 41 | 45.528 | 61.522 | 86.889 | 1.00 | 57.21 | C |
| ATOM | 12820 | CD | PRO | D | 41 | 45.811 | 60.620 | 85.736 | 1.00 | 53.85 | C |
| ATOM | 12823 | C | PRO | D | 41 | 42.753 | 62.260 | 85.443 | 1.00 | 59.94 | C |
| ATOM | 12824 | O | PRO | D | 41 | 41.491 | 62.335 | 85.416 | 1.00 | 60.69 | O |
| ATOM | 12825 | N | GLY | D | 42 | 43.616 | 63.113 | 84.835 | 1.00 | 61.45 | N |
| ATOM | 12827 | CA | GLY | D | 42 | 43.269 | 64.364 | 84.131 | 1.00 | 64.05 | C |
| ATOM | 12830 | C | GLY | D | 42 | 42.483 | 64.364 | 82.805 | 1.00 | 63.41 | C |
| ATOM | 12831 | O | GLY | D | 42 | 41.364 | 64.858 | 82.825 | 1.00 | 65.45 | O |
| ATOM | 12832 | N | LYS | D | 43 | 43.023 | 63.893 | 81.661 | 1.00 | 60.97 | N |
| ATOM | 12834 | CA | LYS | D | 43 | 42.118 | 63.505 | 80.546 | 1.00 | 59.80 | C |
| ATOM | 12836 | CB | LYS | D | 43 | 42.851 | 63.131 | 79.232 | 1.00 | 57.52 | C |
| ATOM | 12843 | C | LYS | D | 43 | 41.186 | 62.352 | 81.078 | 1.00 | 58.39 | C |
| ATOM | 12844 | O | LYS | D | 43 | 40.305 | 62.593 | 82.000 | 1.00 | 63.11 | O |
| ATOM | 12845 | N | GLY | D | 44 | 41.315 | 61.144 | 80.548 | 1.00 | 53.00 | N |
| ATOM | 12847 | CA | GLY | D | 44 | 40.557 | 60.047 | 81.120 | 1.00 | 51.14 | C |
| ATOM | 12850 | C | GLY | D | 44 | 41.027 | 58.728 | 80.530 | 1.00 | 47.33 | C |
| ATOM | 12851 | O | GLY | D | 44 | 42.277 | 58.557 | 80.366 | 1.00 | 46.03 | O |
| ATOM | 12852 | N | LEU | D | 45 | 40.088 | 57.808 | 80.217 | 1.00 | 44.05 | N |
| ATOM | 12854 | CA | LEU | D | 45 | 40.455 | 56.553 | 79.559 | 1.00 | 40.18 | C |
| ATOM | 12856 | CB | LEU | D | 45 | 39.386 | 55.575 | 79.824 | 1.00 | 39.67 | C |
| ATOM | 12859 | CG | LEU | D | 45 | 39.601 | 54.171 | 80.329 | 1.00 | 38.63 | C |
| ATOM | 12861 | CD1 | LEU | D | 45 | 40.898 | 53.853 | 80.863 | 1.00 | 37.61 | C |
| ATOM | 12865 | CD2 | LEU | D | 45 | 38.510 | 54.020 | 81.401 | 1.00 | 42.06 | C |
| ATOM | 12869 | C | LEU | D | 45 | 40.553 | 56.808 | 78.075 | 1.00 | 38.78 | C |
| ATOM | 12870 | O | LEU | D | 45 | 39.585 | 57.217 | 77.474 | 1.00 | 42.37 | O |
| ATOM | 12871 | N | GLU | D | 46 | 41.683 | 56.616 | 77.442 | 1.00 | 36.39 | N |
| ATOM | 12873 | CA | GLU | D | 46 | 41.756 | 56.917 | 76.023 | 1.00 | 36.08 | C |
| ATOM | 12875 | CB | GLU | D | 46 | 42.482 | 58.195 | 75.876 | 1.00 | 38.06 | C |
| ATOM | 12878 | CG | GLU | D | 46 | 42.640 | 58.660 | 74.466 | 1.00 | 43.17 | C |
| ATOM | 12881 | CD | GLU | D | 46 | 43.757 | 59.721 | 74.236 | 1.00 | 49.47 | C |
| ATOM | 12882 | OE1 | GLU | D | 46 | 44.654 | 59.993 | 75.192 | 1.00 | 44.91 | O |
| ATOM | 12883 | OE2 | GLU | D | 46 | 43.686 | 60.248 | 73.037 | 1.00 | 46.97 | O |
| ATOM | 12884 | C | GLU | D | 46 | 42.359 | 55.793 | 75.169 | 1.00 | 32.98 | C |
| ATOM | 12885 | O | GLU | D | 46 | 43.347 | 55.226 | 75.508 | 1.00 | 31.48 | O |
| ATOM | 12886 | N | TRP | D | 47 | 41.679 | 55.438 | 74.096 | 1.00 | 32.53 | N |
| ATOM | 12888 | CA | TRP | D | 47 | 42.154 | 54.391 | 73.191 | 1.00 | 32.16 | C |
| ATOM | 12890 | CB | TRP | D | 47 | 41.001 | 53.746 | 72.467 | 1.00 | 31.08 | C |
| ATOM | 12893 | CG | TRP | D | 47 | 41.254 | 52.697 | 71.439 | 1.00 | 30.69 | C |
| ATOM | 12894 | CD1 | TRP | D | 47 | 41.205 | 51.356 | 71.611 | 1.00 | 30.28 | C |
| ATOM | 12896 | NE1 | TRP | D | 47 | 41.413 | 50.701 | 70.416 | 1.00 | 29.31 | N |
| ATOM | 12898 | CE2 | TRP | D | 47 | 41.587 | 51.627 | 69.432 | 1.00 | 29.06 | C |
| ATOM | 12899 | CD2 | TRP | D | 47 | 41.481 | 52.897 | 70.034 | 1.00 | 33.41 | C |
| ATOM | 12900 | CE3 | TRP | D | 47 | 41.626 | 54.035 | 69.212 | 1.00 | 36.15 | C |
| ATOM | 12902 | CZ3 | TRP | D | 47 | 41.836 | 53.864 | 67.825 | 1.00 | 32.52 | C |
| ATOM | 12904 | CH2 | TRP | D | 47 | 41.916 | 52.607 | 67.285 | 1.00 | 30.16 | C |
| ATOM | 12906 | CZ2 | TRP | D | 47 | 41.785 | 51.466 | 68.079 | 1.00 | 29.39 | C |
| ATOM | 12908 | C | TRP | D | 47 | 43.269 | 54.825 | 72.197 | 1.00 | 32.70 | C |
| ATOM | 12909 | O | TRP | D | 47 | 43.190 | 55.885 | 71.523 | 1.00 | 32.87 | O |
| ATOM | 12910 | N | LEU | D | 48 | 44.322 | 53.995 | 72.178 | 1.00 | 31.73 | N |
| ATOM | 12912 | CA | LEU | D | 48 | 45.556 | 54.317 | 71.463 | 1.00 | 32.99 | C |
| ATOM | 12914 | CB | LEU | D | 48 | 46.776 | 53.933 | 72.280 | 1.00 | 32.64 | C |
| ATOM | 12917 | CG | LEU | D | 48 | 46.758 | 54.648 | 73.611 | 1.00 | 32.13 | C |
| ATOM | 12919 | CD1 | LEU | D | 48 | 47.957 | 54.186 | 74.392 | 1.00 | 32.17 | C |
| ATOM | 12923 | CD2 | LEU | D | 48 | 46.771 | 56.119 | 73.414 | 1.00 | 32.53 | C |
| ATOM | 12927 | C | LEU | D | 48 | 45.631 | 53.665 | 70.121 | 1.00 | 32.70 | C |
| ATOM | 12928 | O | LEU | D | 48 | 45.921 | 54.298 | 69.140 | 1.00 | 32.71 | O |
| ATOM | 12929 | N | GLY | D | 49 | 45.363 | 52.376 | 70.092 | 1.00 | 32.34 | N |
| ATOM | 12931 | CA | GLY | D | 49 | 45.225 | 51.704 | 68.809 | 1.00 | 32.63 | C |
| ATOM | 12934 | C | GLY | D | 49 | 45.081 | 50.210 | 68.962 | 1.00 | 31.13 | C |
| ATOM | 12935 | O | GLY | D | 49 | 44.752 | 49.728 | 70.032 | 1.00 | 30.55 | O |
| ATOM | 12936 | N | VAL | D | 50 | 45.357 | 49.499 | 67.880 | 1.00 | 30.69 | N |
| ATOM | 12938 | CA | VAL | D | 50 | 45.234 | 48.071 | 67.858 | 1.00 | 29.69 | C |
| ATOM | 12940 | CB | VAL | D | 50 | 43.921 | 47.569 | 67.214 | 1.00 | 30.33 | C |
| ATOM | 12942 | CG1 | VAL | D | 50 | 43.191 | 46.659 | 68.168 | 1.00 | 29.69 | C |
| ATOM | 12946 | CG2 | VAL | D | 50 | 43.087 | 48.640 | 66.687 | 1.00 | 30.44 | C |
| ATOM | 12950 | C | VAL | D | 50 | 46.227 | 47.452 | 66.954 | 1.00 | 29.44 | C |
| ATOM | 12951 | O | VAL | D | 50 | 46.492 | 47.957 | 65.895 | 1.00 | 28.43 | O |
| ATOM | 12952 | N | ILE | D | 51 | 46.704 | 46.296 | 67.352 | 1.00 | 29.83 | N |
| ATOM | 12954 | CA | ILE | D | 51 | 47.217 | 45.344 | 66.382 | 1.00 | 31.60 | C |
| ATOM | 12956 | CB | ILE | D | 51 | 48.621 | 44.854 | 66.705 | 1.00 | 32.32 | C |
| ATOM | 12958 | CG1 | ILE | D | 51 | 49.185 | 44.059 | 65.511 | 1.00 | 36.70 | C |
| ATOM | 12961 | CD1 | ILE | D | 51 | 50.707 | 43.620 | 65.590 | 1.00 | 32.82 | C |
| ATOM | 12965 | CG2 | ILE | D | 51 | 48.644 | 43.931 | 67.879 | 1.00 | 32.72 | C |
| ATOM | 12969 | C | ILE | D | 51 | 46.179 | 44.218 | 66.317 | 1.00 | 31.06 | C |
| ATOM | 12970 | O | ILE | D | 51 | 46.002 | 43.445 | 67.231 | 1.00 | 30.75 | O |
| ATOM | 12971 | N | TRP | D | 52 | 45.429 | 44.247 | 65.242 | 1.00 | 31.60 | N |
| ATOM | 12973 | CA | TRP | D | 52 | 44.467 | 43.230 | 64.869 | 1.00 | 31.36 | C |
| ATOM | 12975 | CB | TRP | D | 52 | 43.712 | 43.639 | 63.636 | 1.00 | 30.90 | C |
| ATOM | 12978 | CG | TRP | D | 52 | 42.953 | 44.914 | 63.709 | 1.00 | 29.08 | C |
| ATOM | 12979 | CD1 | TRP | D | 52 | 43.219 | 46.097 | 63.033 | 1.00 | 28.04 | C |
| ATOM | 12981 | NE1 | TRP | D | 52 | 42.243 | 47.006 | 63.289 | 1.00 | 26.54 | N |
| ATOM | 12983 | CE2 | TRP | D | 52 | 41.321 | 46.397 | 64.086 | 1.00 | 25.66 | C |
| ATOM | 12984 | CD2 | TRP | D | 52 | 41.738 | 45.097 | 64.333 | 1.00 | 26.15 | C |
| ATOM | 12985 | CE3 | TRP | D | 52 | 40.927 | 44.278 | 65.103 | 1.00 | 29.06 | C |
| ATOM | 12987 | CZ3 | TRP | D | 52 | 39.779 | 44.803 | 65.625 | 1.00 | 26.64 | C |
| ATOM | 12989 | CH2 | TRP | D | 52 | 39.410 | 46.068 | 65.353 | 1.00 | 27.20 | C |
| ATOM | 12991 | CZ2 | TRP | D | 52 | 40.166 | 46.880 | 64.583 | 1.00 | 28.98 | C |
| ATOM | 12993 | C | TRP | D | 52 | 45.108 | 41.895 | 64.531 | 1.00 | 32.27 | C |
| ATOM | 12994 | O | TRP | D | 52 | 46.307 | 41.779 | 64.320 | 1.00 | 32.72 | O |
| ATOM | 12995 | N | SER | D | 53 | 44.227 | 40.915 | 64.488 | 1.00 | 32.74 | N |
| ATOM | 12997 | CA | SER | D | 53 | 44.580 | 39.525 | 64.425 | 1.00 | 34.75 | C |
| ATOM | 12999 | CB | SER | D | 53 | 43.298 | 38.681 | 64.426 | 1.00 | 35.90 | C |
| ATOM | 13002 | OG | SER | D | 53 | 42.764 | 38.516 | 63.151 | 1.00 | 37.74 | O |
| ATOM | 13004 | C | SER | D | 53 | 45.454 | 39.204 | 63.274 | 1.00 | 35.30 | C |
| ATOM | 13005 | O | SER | D | 53 | 46.503 | 38.646 | 63.451 | 1.00 | 37.19 | O |
| ATOM | 13006 | N | GLY | D | 54 | 45.092 | 39.637 | 62.083 | 1.00 | 35.49 | N |
| ATOM | 13008 | CA | GLY | D | 54 | 45.992 | 39.425 | 60.943 | 1.00 | 35.48 | C |
| ATOM | 13011 | C | GLY | D | 54 | 47.220 | 40.303 | 60.835 | 1.00 | 34.39 | C |
| ATOM | 13012 | O | GLY | D | 54 | 47.807 | 40.343 | 59.768 | 1.00 | 36.25 | O |
| ATOM | 13013 | N | GLY | D | 55 | 47.614 | 40.991 | 61.899 | 1.00 | 32.23 | N |
| ATOM | 13015 | CA | GLY | D | 55 | 48.901 | 41.684 | 61.913 | 1.00 | 33.48 | C |
| ATOM | 13018 | C | GLY | D | 55 | 48.859 | 43.138 | 61.531 | 1.00 | 33.28 | C |
| ATOM | 13019 | O | GLY | D | 55 | 49.752 | 43.862 | 61.701 | 1.00 | 33.23 | O |
| ATOM | 13020 | N | ASN | D | 56 | 47.752 | 43.574 | 61.012 | 1.00 | 33.55 | N |
| ATOM | 13022 | CA | ASN | D | 56 | 47.664 | 44.924 | 60.574 | 1.00 | 35.11 | C |
| ATOM | 13024 | CB | ASN | D | 56 | 46.626 | 45.025 | 59.499 | 1.00 | 34.70 | C |
| ATOM | 13027 | CG | ASN | D | 56 | 45.305 | 44.633 | 59.959 | 1.00 | 35.37 | C |
| ATOM | 13028 | OD1 | ASN | D | 56 | 44.385 | 45.451 | 59.883 | 1.00 | 40.28 | O |
| ATOM | 13029 | ND2 | ASN | D | 56 | 45.122 | 43.357 | 60.343 | 1.00 | 36.56 | N |
| ATOM | 13032 | C | ASN | D | 56 | 47.347 | 45.795 | 61.765 | 1.00 | 34.36 | C |
| ATOM | 13033 | O | ASN | D | 56 | 46.820 | 45.268 | 62.750 | 1.00 | 33.78 | O |
| ATOM | 13034 | N | THR | D | 57 | 47.669 | 47.100 | 61.710 | 1.00 | 34.41 | N |
| ATOM | 13036 | CA | THR | D | 57 | 47.319 | 47.969 | 62.842 | 1.00 | 32.78 | C |
| ATOM | 13038 | CB | THR | D | 57 | 48.563 | 48.487 | 63.562 | 1.00 | 32.50 | C |
| ATOM | 13040 | OG1 | THR | D | 57 | 49.303 | 49.334 | 62.711 | 1.00 | 34.26 | O |
| ATOM | 13042 | CG2 | THR | D | 57 | 49.505 | 47.392 | 63.858 | 1.00 | 33.15 | C |
| ATOM | 13046 | C | THR | D | 57 | 46.377 | 49.127 | 62.532 | 1.00 | 32.96 | C |
| ATOM | 13047 | O | THR | D | 57 | 46.016 | 49.401 | 61.417 | 1.00 | 34.56 | O |
| ATOM | 13048 | N | ASP | D | 58 | 45.957 | 49.794 | 63.572 | 1.00 | 32.56 | N |
| ATOM | 13050 | CA | ASP | D | 58 | 45.262 | 51.043 | 63.418 | 1.00 | 33.53 | C |
| ATOM | 13052 | CB | ASP | D | 58 | 43.805 | 50.801 | 63.352 | 1.00 | 32.81 | C |
| ATOM | 13055 | CG | ASP | D | 58 | 43.086 | 51.930 | 62.805 | 1.00 | 35.44 | C |
| ATOM | 13056 | OD1 | ASP | D | 58 | 43.702 | 52.786 | 62.201 | 1.00 | 39.99 | O |
| ATOM | 13057 | OD2 | ASP | D | 58 | 41.850 | 52.052 | 62.880 | 1.00 | 44.18 | O |
| ATOM | 13058 | C | ASP | D | 58 | 45.555 | 51.858 | 64.639 | 1.00 | 34.20 | C |
| ATOM | 13059 | O | ASP | D | 58 | 45.601 | 51.348 | 65.789 | 1.00 | 33.15 | O |
| ATOM | 13060 | N | TYR | D | 59 | 45.757 | 53.139 | 64.413 | 1.00 | 36.15 | N |
| ATOM | 13062 | CA | TYR | D | 59 | 46.178 | 54.024 | 65.491 | 1.00 | 35.91 | C |
| ATOM | 13064 | CB | TYR | D | 59 | 47.573 | 54.426 | 65.247 | 1.00 | 36.57 | C |
| ATOM | 13067 | CG | TYR | D | 59 | 48.572 | 53.328 | 65.088 | 1.00 | 37.04 | C |
| ATOM | 13068 | CD1 | TYR | D | 59 | 49.590 | 53.425 | 64.103 | 1.00 | 40.41 | C |
| ATOM | 13070 | CE1 | TYR | D | 59 | 50.555 | 52.505 | 63.961 | 1.00 | 38.26 | C |
| ATOM | 13072 | CZ | TYR | D | 59 | 50.592 | 51.439 | 64.807 | 1.00 | 36.55 | C |
| ATOM | 13073 | OH | TYR | D | 59 | 51.546 | 50.524 | 64.623 | 1.00 | 38.64 | O |
| ATOM | 13075 | CE2 | TYR | D | 59 | 49.668 | 51.275 | 65.780 | 1.00 | 36.05 | C |
| ATOM | 13077 | CD2 | TYR | D | 59 | 48.620 | 52.260 | 65.935 | 1.00 | 36.55 | C |
| ATOM | 13079 | C | TYR | D | 59 | 45.335 | 55.284 | 65.529 | 1.00 | 36.81 | C |
| ATOM | 13080 | O | TYR | D | 59 | 45.092 | 55.899 | 64.524 | 1.00 | 38.49 | O |
| ATOM | 13081 | N | ASN | D | 60 | 44.888 | 55.660 | 66.710 | 1.00 | 36.57 | N |
| ATOM | 13083 | CA | ASN | D | 60 | 44.107 | 56.894 | 66.906 | 1.00 | 38.17 | C |
| ATOM | 13085 | CB | ASN | D | 60 | 43.885 | 57.030 | 68.383 | 1.00 | 37.59 | C |
| ATOM | 13088 | CG | ASN | D | 60 | 42.742 | 57.873 | 68.742 | 1.00 | 39.27 | C |
| ATOM | 13089 | OD1 | ASN | D | 60 | 42.025 | 58.492 | 67.928 | 1.00 | 42.52 | O |
| ATOM | 13090 | ND2 | ASN | D | 60 | 42.533 | 57.895 | 70.029 | 1.00 | 40.55 | N |
| ATOM | 13093 | C | ASN | D | 60 | 44.905 | 58.075 | 66.418 | 1.00 | 39.85 | C |
| ATOM | 13094 | O | ASN | D | 60 | 46.085 | 58.121 | 66.619 | 1.00 | 40.65 | O |
| ATOM | 13095 | N | THR | D | 61 | 44.255 | 59.004 | 65.783 | 1.00 | 41.38 | N |
| ATOM | 13097 | CA | THR | D | 61 | 44.885 | 60.081 | 65.089 | 1.00 | 45.06 | C |
| ATOM | 13099 | CB | THR | D | 61 | 43.838 | 61.144 | 64.787 | 1.00 | 48.42 | C |
| ATOM | 13101 | OG1 | THR | D | 61 | 42.731 | 60.516 | 64.121 | 1.00 | 47.87 | O |
| ATOM | 13103 | CG2 | THR | D | 61 | 44.366 | 62.233 | 63.789 | 1.00 | 52.40 | C |
| ATOM | 13107 | C | THR | D | 61 | 46.027 | 60.733 | 65.763 | 1.00 | 46.36 | C |
| ATOM | 13108 | O | THR | D | 61 | 47.011 | 60.937 | 65.152 | 1.00 | 48.83 | O |
| ATOM | 13109 | N | PRO | D | 62 | 45.936 | 61.148 | 66.995 | 1.00 | 47.67 | N |
| ATOM | 13110 | CA | PRO | D | 62 | 47.106 | 61.814 | 67.621 | 1.00 | 49.35 | C |
| ATOM | 13112 | CB | PRO | D | 62 | 46.538 | 62.388 | 68.924 | 1.00 | 49.55 | C |
| ATOM | 13115 | CG | PRO | D | 62 | 45.387 | 61.448 | 69.263 | 1.00 | 47.57 | C |
| ATOM | 13118 | CD | PRO | D | 62 | 44.769 | 61.089 | 67.900 | 1.00 | 47.33 | C |
| ATOM | 13121 | C | PRO | D | 62 | 48.301 | 60.936 | 67.935 | 1.00 | 47.56 | C |
| ATOM | 13122 | O | PRO | D | 62 | 49.297 | 61.412 | 68.435 | 1.00 | 49.85 | O |
| ATOM | 13123 | N | PHE | D | 63 | 48.225 | 59.660 | 67.661 | 1.00 | 45.89 | N |
| ATOM | 13125 | CA | PHE | D | 63 | 49.354 | 58.776 | 67.922 | 1.00 | 45.48 | C |
| ATOM | 13127 | CB | PHE | D | 63 | 48.991 | 57.694 | 69.022 | 1.00 | 42.86 | C |
| ATOM | 13130 | CG | PHE | D | 63 | 48.348 | 58.264 | 70.261 | 1.00 | 41.77 | C |
| ATOM | 13131 | CD1 | PHE | D | 63 | 46.985 | 58.211 | 70.441 | 1.00 | 37.08 | C |
| ATOM | 13133 | CE1 | PHE | D | 63 | 46.398 | 58.790 | 71.575 | 1.00 | 38.67 | C |
| ATOM | 13135 | CZ | PHE | D | 63 | 47.145 | 59.418 | 72.527 | 1.00 | 37.15 | C |
| ATOM | 13137 | CE2 | PHE | D | 63 | 48.487 | 59.515 | 72.346 | 1.00 | 41.21 | C |
| ATOM | 13139 | CD2 | PHE | D | 63 | 49.109 | 58.935 | 71.211 | 1.00 | 41.81 | C |
| ATOM | 13141 | C | PHE | D | 63 | 49.802 | 58.127 | 66.601 | 1.00 | 46.08 | C |
| ATOM | 13142 | O | PHE | D | 63 | 50.622 | 57.230 | 66.587 | 1.00 | 46.42 | O |
| ATOM | 13143 | N | THR | D | 64 | 49.300 | 58.568 | 65.480 | 1.00 | 47.62 | N |
| ATOM | 13145 | CA | THR | D | 64 | 49.712 | 57.961 | 64.240 | 1.00 | 49.13 | C |
| ATOM | 13147 | CB | THR | D | 64 | 48.972 | 58.633 | 63.160 | 1.00 | 51.36 | C |
| ATOM | 13149 | OG1 | THR | D | 64 | 47.638 | 58.085 | 63.156 | 1.00 | 50.94 | O |
| ATOM | 13151 | CG2 | THR | D | 64 | 49.591 | 58.313 | 61.782 | 1.00 | 54.91 | C |
| ATOM | 13155 | C | THR | D | 64 | 51.210 | 58.114 | 63.990 | 1.00 | 52.42 | C |
| ATOM | 13156 | O | THR | D | 64 | 51.907 | 57.193 | 63.532 | 1.00 | 53.42 | O |
| ATOM | 13157 | N | SER | D | 65 | 51.737 | 59.289 | 64.303 | 1.00 | 54.78 | N |
| ATOM | 13159 | CA | SER | D | 65 | 53.053 | 59.604 | 63.833 | 1.00 | 56.59 | C |
| ATOM | 13161 | CB | SER | D | 65 | 53.251 | 61.158 | 63.765 | 1.00 | 60.91 | C |
| ATOM | 13164 | OG | SER | D | 65 | 53.283 | 61.827 | 65.066 | 1.00 | 60.68 | O |
| ATOM | 13166 | C | SER | D | 65 | 54.083 | 58.943 | 64.716 | 1.00 | 54.95 | C |
| ATOM | 13167 | O | SER | D | 65 | 55.308 | 59.151 | 64.511 | 1.00 | 58.96 | O |
| ATOM | 13168 | N | ARG | D | 66 | 53.650 | 58.240 | 65.753 | 1.00 | 50.49 | N |
| ATOM | 13170 | CA | ARG | D | 66 | 54.617 | 57.976 | 66.852 | 1.00 | 50.01 | C |
| ATOM | 13172 | CB | ARG | D | 66 | 54.716 | 59.163 | 67.832 | 1.00 | 50.28 | C |
| ATOM | 13175 | CG | ARG | D | 66 | 53.473 | 59.338 | 68.681 | 1.00 | 49.71 | C |
| ATOM | 13178 | CD | ARG | D | 66 | 53.310 | 60.748 | 69.279 | 1.00 | 52.47 | C |
| ATOM | 13181 | NE | ARG | D | 66 | 53.856 | 60.823 | 70.640 | 1.00 | 51.78 | N |
| ATOM | 13183 | CZ | ARG | D | 66 | 53.150 | 60.998 | 71.750 | 1.00 | 50.52 | C |
| ATOM | 13184 | NH1 | ARG | D | 66 | 51.848 | 61.166 | 71.744 | 1.00 | 52.35 | N |
| ATOM | 13187 | NH2 | ARG | D | 66 | 53.760 | 61.064 | 72.902 | 1.00 | 52.82 | N |
| ATOM | 13190 | C | ARG | D | 66 | 54.356 | 56.697 | 67.597 | 1.00 | 46.69 | C |
| ATOM | 13191 | O | ARG | D | 66 | 55.145 | 56.247 | 68.434 | 1.00 | 46.16 | O |
| ATOM | 13192 | N | LEU | D | 67 | 53.265 | 56.067 | 67.250 | 1.00 | 44.55 | N |
| ATOM | 13194 | CA | LEU | D | 67 | 52.930 | 54.840 | 67.913 | 1.00 | 42.69 | C |
| ATOM | 13196 | CB | LEU | D | 67 | 51.444 | 54.918 | 68.365 | 1.00 | 40.49 | C |
| ATOM | 13199 | CG | LEU | D | 67 | 50.806 | 53.736 | 69.049 | 1.00 | 39.68 | C |
| ATOM | 13201 | CD1 | LEU | D | 67 | 51.766 | 53.114 | 70.010 | 1.00 | 41.91 | C |
| ATOM | 13205 | CD2 | LEU | D | 67 | 49.535 | 54.112 | 69.731 | 1.00 | 39.30 | C |
| ATOM | 13209 | C | LEU | D | 67 | 53.301 | 53.694 | 66.939 | 1.00 | 42.23 | C |
| ATOM | 13210 | O | LEU | D | 67 | 53.187 | 53.803 | 65.722 | 1.00 | 41.99 | O |
| ATOM | 13211 | N | SER | D | 68 | 53.814 | 52.615 | 67.484 | 1.00 | 41.73 | N |
| ATOM | 13213 | CA | SER | D | 68 | 53.956 | 51.426 | 66.679 | 1.00 | 42.24 | C |
| ATOM | 13215 | CB | SER | D | 68 | 55.369 | 51.322 | 66.097 | 1.00 | 44.65 | C |
| ATOM | 13218 | OG | SER | D | 68 | 56.007 | 50.216 | 66.719 | 1.00 | 44.64 | O |
| ATOM | 13220 | C | SER | D | 68 | 53.670 | 50.202 | 67.537 | 1.00 | 40.14 | C |
| ATOM | 13221 | O | SER | D | 68 | 54.406 | 49.959 | 68.458 | 1.00 | 41.75 | O |
| ATOM | 13222 | N | ILE | D | 69 | 52.646 | 49.430 | 67.192 | 1.00 | 38.16 | N |
| ATOM | 13224 | CA | ILE | D | 69 | 52.444 | 48.115 | 67.777 | 1.00 | 37.76 | C |
| ATOM | 13226 | CB | ILE | D | 69 | 51.042 | 47.962 | 68.094 | 1.00 | 35.22 | C |
| ATOM | 13228 | CG1 | ILE | D | 69 | 50.542 | 49.253 | 68.763 | 1.00 | 35.84 | C |
| ATOM | 13231 | CD1 | ILE | D | 69 | 49.059 | 49.374 | 69.024 | 1.00 | 34.14 | C |
| ATOM | 13235 | CG2 | ILE | D | 69 | 50.941 | 46.834 | 69.044 | 1.00 | 36.27 | C |
| ATOM | 13239 | C | ILE | D | 69 | 52.930 | 46.915 | 66.909 | 1.00 | 39.46 | C |
| ATOM | 13240 | O | ILE | D | 69 | 52.799 | 46.952 | 65.673 | 1.00 | 41.51 | O |
| ATOM | 13241 | N | ASN | D | 70 | 53.501 | 45.877 | 67.523 | 1.00 | 39.23 | N |
| ATOM | 13243 | CA | ASN | D | 70 | 53.977 | 44.715 | 66.762 | 1.00 | 41.63 | C |
| ATOM | 13245 | CB | ASN | D | 70 | 55.460 | 44.779 | 66.533 | 1.00 | 44.84 | C |
| ATOM | 13248 | CG | ASN | D | 70 | 55.800 | 45.718 | 65.434 | 1.00 | 49.17 | C |
| ATOM | 13249 | OD1 | ASN | D | 70 | 55.575 | 45.399 | 64.289 | 1.00 | 55.81 | O |
| ATOM | 13250 | ND2 | ASN | D | 70 | 56.256 | 46.927 | 65.763 | 1.00 | 52.56 | N |
| ATOM | 13253 | C | ASN | D | 70 | 53.631 | 43.453 | 67.469 | 1.00 | 40.86 | C |
| ATOM | 13254 | O | ASN | D | 70 | 53.133 | 43.517 | 68.548 | 1.00 | 40.73 | O |
| ATOM | 13255 | N | LYS | D | 71 | 53.824 | 42.299 | 66.872 | 1.00 | 41.57 | N |
| ATOM | 13257 | CA | LYS | D | 71 | 53.571 | 41.114 | 67.652 | 1.00 | 41.77 | C |
| ATOM | 13259 | CB | LYS | D | 71 | 52.089 | 40.784 | 67.647 | 1.00 | 39.31 | C |
| ATOM | 13262 | CG | LYS | D | 71 | 51.532 | 40.227 | 66.385 | 1.00 | 38.04 | C |
| ATOM | 13265 | CD | LYS | D | 71 | 49.962 | 40.233 | 66.402 | 1.00 | 37.00 | C |
| ATOM | 13268 | CE | LYS | D | 71 | 49.309 | 39.359 | 65.314 | 1.00 | 34.15 | C |
| ATOM | 13271 | NZ | LYS | D | 71 | 47.935 | 39.378 | 65.457 | 1.00 | 32.61 | N |
| ATOM | 13275 | C | LYS | D | 71 | 54.304 | 39.898 | 67.162 | 1.00 | 45.31 | C |
| ATOM | 13276 | O | LYS | D | 71 | 54.644 | 39.819 | 66.008 | 1.00 | 47.02 | O |
| ATOM | 13277 | N | ASP | D | 72 | 54.499 | 38.940 | 68.061 | 1.00 | 46.92 | N |
| ATOM | 13279 | CA | ASP | D | 72 | 54.911 | 37.579 | 67.731 | 1.00 | 49.86 | C |
| ATOM | 13281 | CB | ASP | D | 72 | 56.252 | 37.312 | 68.418 | 1.00 | 52.20 | C |
| ATOM | 13284 | CG | ASP | D | 72 | 56.857 | 36.047 | 67.966 | 1.00 | 58.26 | C |
| ATOM | 13285 | OD1 | ASP | D | 72 | 56.033 | 35.215 | 67.531 | 1.00 | 59.63 | O |
| ATOM | 13286 | OD2 | ASP | D | 72 | 58.108 | 35.774 | 67.965 | 1.00 | 67.49 | O |
| ATOM | 13287 | C | ASP | D | 72 | 53.816 | 36.496 | 68.102 | 1.00 | 48.55 | C |
| ATOM | 13288 | O | ASP | D | 72 | 53.706 | 36.125 | 69.270 | 1.00 | 48.20 | O |
| ATOM | 13289 | N | ASN | D | 73 | 53.015 | 36.002 | 67.142 | 1.00 | 47.65 | N |
| ATOM | 13291 | CA | ASN | D | 73 | 51.975 | 35.023 | 67.542 | 1.00 | 47.21 | C |
| ATOM | 13293 | CB | ASN | D | 73 | 50.978 | 34.570 | 66.476 | 1.00 | 45.48 | C |
| ATOM | 13296 | CG | ASN | D | 73 | 50.327 | 35.684 | 65.713 | 1.00 | 43.16 | C |
| ATOM | 13297 | OD1 | ASN | D | 73 | 49.630 | 36.539 | 66.293 | 1.00 | 47.44 | O |
| ATOM | 13298 | ND2 | ASN | D | 73 | 50.408 | 35.612 | 64.406 | 1.00 | 39.25 | N |
| ATOM | 13301 | C | ASN | D | 73 | 52.598 | 33.748 | 68.083 | 1.00 | 51.12 | C |
| ATOM | 13302 | O | ASN | D | 73 | 51.987 | 33.107 | 68.883 | 1.00 | 52.43 | O |
| ATOM | 13303 | N | SER | D | 74 | 53.773 | 33.318 | 67.643 | 1.00 | 54.87 | N |
| ATOM | 13305 | CA | SER | D | 74 | 54.397 | 32.122 | 68.317 | 1.00 | 58.74 | C |
| ATOM | 13307 | CB | SER | D | 74 | 55.773 | 31.817 | 67.787 | 1.00 | 62.13 | C |
| ATOM | 13310 | OG | SER | D | 74 | 55.629 | 31.667 | 66.404 | 1.00 | 65.94 | O |
| ATOM | 13312 | C | SER | D | 74 | 54.543 | 32.307 | 69.802 | 1.00 | 58.18 | C |
| ATOM | 13313 | O | SER | D | 74 | 54.129 | 31.462 | 70.531 | 1.00 | 60.18 | O |
| ATOM | 13314 | N | LYS | D | 75 | 55.131 | 33.421 | 70.238 | 1.00 | 56.43 | N |
| ATOM | 13316 | CA | LYS | D | 75 | 55.368 | 33.665 | 71.639 | 1.00 | 56.60 | C |
| ATOM | 13318 | CB | LYS | D | 75 | 56.566 | 34.600 | 71.806 | 1.00 | 57.02 | C |
| ATOM | 13321 | CG | LYS | D | 75 | 57.816 | 33.999 | 71.324 | 1.00 | 62.21 | C |
| ATOM | 13324 | CD | LYS | D | 75 | 58.969 | 34.990 | 71.079 | 1.00 | 65.08 | C |
| ATOM | 13327 | CE | LYS | D | 75 | 60.071 | 34.308 | 70.161 | 1.00 | 69.37 | C |
| ATOM | 13330 | NZ | LYS | D | 75 | 61.327 | 33.664 | 70.861 | 1.00 | 75.58 | N |
| ATOM | 13334 | C | LYS | D | 75 | 54.153 | 34.295 | 72.277 | 1.00 | 53.67 | C |
| ATOM | 13335 | O | LYS | D | 75 | 54.254 | 34.800 | 73.361 | 1.00 | 53.73 | O |
| ATOM | 13336 | N | SER | D | 76 | 52.997 | 34.249 | 71.621 | 1.00 | 52.05 | N |
| ATOM | 13338 | CA | SER | D | 76 | 51.836 | 35.122 | 71.941 | 1.00 | 48.62 | C |
| ATOM | 13340 | CB | SER | D | 76 | 50.739 | 34.328 | 72.636 | 1.00 | 48.74 | C |
| ATOM | 13343 | OG | SER | D | 76 | 51.376 | 33.677 | 73.649 | 1.00 | 52.34 | O |
| ATOM | 13345 | C | SER | D | 76 | 52.224 | 36.397 | 72.744 | 1.00 | 46.65 | C |
| ATOM | 13346 | O | SER | D | 76 | 51.862 | 36.571 | 73.915 | 1.00 | 45.12 | O |
| ATOM | 13347 | N | GLN | D | 77 | 52.938 | 37.285 | 72.056 | 1.00 | 45.21 | N |
| ATOM | 13349 | CA | GLN | D | 77 | 53.433 | 38.479 | 72.656 | 1.00 | 43.66 | C |
| ATOM | 13351 | CB | GLN | D | 77 | 54.970 | 38.339 | 72.814 | 1.00 | 46.88 | C |
| ATOM | 13354 | CG | GLN | D | 77 | 55.413 | 37.830 | 74.223 | 1.00 | 47.53 | C |
| ATOM | 13357 | CD | GLN | D | 77 | 56.927 | 37.700 | 74.397 | 1.00 | 49.35 | C |
| ATOM | 13358 | OE1 | GLN | D | 77 | 57.662 | 37.900 | 73.452 | 1.00 | 49.14 | O |
| ATOM | 13359 | NE2 | GLN | D | 77 | 57.377 | 37.335 | 75.607 | 1.00 | 49.80 | N |
| ATOM | 13362 | C | GLN | D | 77 | 53.046 | 39.686 | 71.801 | 1.00 | 40.90 | C |
| ATOM | 13363 | O | GLN | D | 77 | 53.118 | 39.637 | 70.599 | 1.00 | 39.78 | O |
| ATOM | 13364 | N | VAL | D | 78 | 52.621 | 40.767 | 72.441 | 1.00 | 39.44 | N |
| ATOM | 13366 | CA | VAL | D | 78 | 52.391 | 42.018 | 71.747 | 1.00 | 38.83 | C |
| ATOM | 13368 | CB | VAL | D | 78 | 51.002 | 42.480 | 71.899 | 1.00 | 36.73 | C |
| ATOM | 13370 | CG1 | VAL | D | 78 | 50.881 | 43.889 | 71.317 | 1.00 | 36.47 | C |
| ATOM | 13374 | CG2 | VAL | D | 78 | 50.060 | 41.533 | 71.152 | 1.00 | 36.98 | C |
| ATOM | 13378 | C | VAL | D | 78 | 53.269 | 43.132 | 72.259 | 1.00 | 39.68 | C |
| ATOM | 13379 | O | VAL | D | 78 | 53.417 | 43.313 | 73.430 | 1.00 | 41.11 | O |
| ATOM | 13380 | N | PHE | D | 79 | 53.852 | 43.887 | 71.350 | 1.00 | 41.21 | N |
| ATOM | 13382 | CA | PHE | D | 79 | 54.922 | 44.885 | 71.657 | 1.00 | 42.91 | C |
| ATOM | 13384 | CB | PHE | D | 79 | 56.198 | 44.576 | 70.873 | 1.00 | 44.00 | C |
| ATOM | 13387 | CG | PHE | D | 79 | 56.685 | 43.209 | 71.048 | 1.00 | 44.41 | C |
| ATOM | 13388 | CD1 | PHE | D | 79 | 56.809 | 42.361 | 69.986 | 1.00 | 44.33 | C |
| ATOM | 13390 | CE1 | PHE | D | 79 | 57.312 | 41.097 | 70.146 | 1.00 | 44.84 | C |
| ATOM | 13392 | CZ | PHE | D | 79 | 57.692 | 40.655 | 71.360 | 1.00 | 46.86 | C |
| ATOM | 13394 | CE2 | PHE | D | 79 | 57.625 | 41.481 | 72.428 | 1.00 | 47.48 | C |
| ATOM | 13396 | CD2 | PHE | D | 79 | 57.110 | 42.779 | 72.274 | 1.00 | 47.39 | C |
| ATOM | 13398 | C | PHE | D | 79 | 54.451 | 46.315 | 71.284 | 1.00 | 41.27 | C |
| ATOM | 13399 | O | PHE | D | 79 | 54.445 | 46.684 | 70.135 | 1.00 | 41.52 | O |
| ATOM | 13400 | N | PHE | D | 80 | 54.052 | 47.086 | 72.266 | 1.00 | 40.35 | N |
| ATOM | 13402 | CA | PHE | D | 80 | 53.562 | 48.422 | 72.037 | 1.00 | 39.60 | C |
| ATOM | 13404 | CB | PHE | D | 80 | 52.521 | 48.721 | 73.090 | 1.00 | 38.21 | C |
| ATOM | 13407 | CG | PHE | D | 80 | 52.216 | 50.165 | 73.261 | 1.00 | 37.90 | C |
| ATOM | 13408 | CD1 | PHE | D | 80 | 51.021 | 50.680 | 72.821 | 1.00 | 35.78 | C |
| ATOM | 13410 | CE1 | PHE | D | 80 | 50.725 | 51.977 | 73.032 | 1.00 | 34.24 | C |
| ATOM | 13412 | CZ | PHE | D | 80 | 51.553 | 52.761 | 73.706 | 1.00 | 33.92 | C |
| ATOM | 13414 | CE2 | PHE | D | 80 | 52.672 | 52.283 | 74.183 | 1.00 | 37.81 | C |
| ATOM | 13416 | CD2 | PHE | D | 80 | 53.045 | 50.982 | 73.964 | 1.00 | 37.72 | C |
| ATOM | 13418 | C | PHE | D | 80 | 54.725 | 49.303 | 72.235 | 1.00 | 41.55 | C |
| ATOM | 13419 | O | PHE | D | 80 | 55.430 | 49.157 | 73.215 | 1.00 | 42.98 | O |
| ATOM | 13420 | N | LYS | D | 81 | 54.956 | 50.208 | 71.309 | 1.00 | 42.00 | N |
| ATOM | 13422 | CA | LYS | D | 81 | 56.094 | 51.126 | 71.444 | 1.00 | 43.52 | C |
| ATOM | 13424 | CB | LYS | D | 81 | 57.243 | 50.693 | 70.573 | 1.00 | 45.25 | C |
| ATOM | 13427 | CG | LYS | D | 81 | 58.519 | 51.595 | 70.732 | 1.00 | 48.35 | C |
| ATOM | 13430 | CD | LYS | D | 81 | 59.776 | 51.198 | 69.903 | 1.00 | 49.70 | C |
| ATOM | 13433 | CE | LYS | D | 81 | 60.773 | 52.392 | 69.816 | 1.00 | 53.14 | C |
| ATOM | 13436 | NZ | LYS | D | 81 | 62.159 | 52.008 | 69.543 | 1.00 | 55.94 | N |
| ATOM | 13440 | C | LYS | D | 81 | 55.604 | 52.514 | 71.033 | 1.00 | 43.91 | C |
| ATOM | 13441 | O | LYS | D | 81 | 54.940 | 52.640 | 69.988 | 1.00 | 44.59 | O |
| ATOM | 13442 | N | MET | D | 82 | 55.837 | 53.535 | 71.861 | 1.00 | 44.38 | N |
| ATOM | 13444 | CA | MET | D | 82 | 55.448 | 54.904 | 71.496 | 1.00 | 45.09 | C |
| ATOM | 13446 | CB | MET | D | 82 | 54.343 | 55.408 | 72.393 | 1.00 | 44.28 | C |
| ATOM | 13449 | CG | MET | D | 82 | 54.024 | 56.895 | 72.173 | 1.00 | 45.85 | C |
| ATOM | 13452 | SD | MET | D | 82 | 52.329 | 57.187 | 72.824 | 1.00 | 47.44 | S |
| ATOM | 13453 | CE | MET | D | 82 | 52.586 | 57.665 | 74.489 | 1.00 | 46.84 | C |
| ATOM | 13457 | C | MET | D | 82 | 56.604 | 55.815 | 71.643 | 1.00 | 47.35 | C |
| ATOM | 13458 | O | MET | D | 82 | 57.299 | 55.728 | 72.615 | 1.00 | 47.79 | O |
| ATOM | 13459 | N | ASN | D | 83 | 56.792 | 56.728 | 70.704 | 1.00 | 49.56 | N |
| ATOM | 13461 | CA | ASN | D | 83 | 58.009 | 57.562 | 70.660 | 1.00 | 52.90 | C |
| ATOM | 13463 | CB | ASN | D | 83 | 58.447 | 57.777 | 69.202 | 1.00 | 55.93 | C |
| ATOM | 13466 | CG | ASN | D | 83 | 58.932 | 56.492 | 68.511 | 1.00 | 58.70 | C |
| ATOM | 13467 | OD1 | ASN | D | 83 | 59.525 | 55.600 | 69.150 | 1.00 | 60.92 | O |
| ATOM | 13468 | ND2 | ASN | D | 83 | 58.724 | 56.428 | 67.175 | 1.00 | 62.10 | N |
| ATOM | 13471 | C | ASN | D | 83 | 57.828 | 58.935 | 71.279 | 1.00 | 53.16 | C |
| ATOM | 13472 | O | ASN | D | 83 | 56.723 | 59.418 | 71.428 | 1.00 | 50.57 | O |
| ATOM | 13473 | N | SER | D | 84 | 58.938 | 59.578 | 71.592 | 1.00 | 56.13 | N |
| ATOM | 13475 | CA | SER | D | 84 | 58.927 | 60.981 | 72.007 | 1.00 | 58.85 | C |
| ATOM | 13477 | CB | SER | D | 84 | 58.743 | 61.821 | 70.818 | 1.00 | 60.49 | C |
| ATOM | 13480 | OG | SER | D | 84 | 59.650 | 61.296 | 69.902 | 1.00 | 66.30 | O |
| ATOM | 13482 | C | SER | D | 84 | 57.859 | 61.347 | 72.991 | 1.00 | 57.53 | C |
| ATOM | 13483 | O | SER | D | 84 | 56.903 | 62.028 | 72.657 | 1.00 | 57.77 | O |
| ATOM | 13484 | N | LEU | D | 85 | 58.044 | 60.895 | 74.219 | 1.00 | 56.64 | N |
| ATOM | 13486 | CA | LEU | D | 85 | 57.070 | 61.114 | 75.227 | 1.00 | 55.13 | C |
| ATOM | 13488 | CB | LEU | D | 85 | 57.161 | 60.009 | 76.274 | 1.00 | 53.82 | C |
| ATOM | 13491 | CG | LEU | D | 85 | 56.234 | 58.846 | 75.910 | 1.00 | 50.98 | C |
| ATOM | 13493 | CD1 | LEU | D | 85 | 56.953 | 57.991 | 74.900 | 1.00 | 52.48 | C |
| ATOM | 13497 | CD2 | LEU | D | 85 | 55.847 | 58.046 | 77.151 | 1.00 | 49.77 | C |
| ATOM | 13501 | C | LEU | D | 85 | 57.320 | 62.484 | 75.826 | 1.00 | 57.61 | C |
| ATOM | 13502 | O | LEU | D | 85 | 58.423 | 62.985 | 75.802 | 1.00 | 60.21 | O |
| ATOM | 13503 | N | GLN | D | 86 | 56.247 | 63.093 | 76.298 | 1.00 | 56.61 | N |
| ATOM | 13505 | CA | GLN | D | 86 | 56.287 | 64.342 | 77.012 | 1.00 | 58.88 | C |
| ATOM | 13507 | CB | GLN | D | 86 | 55.661 | 65.449 | 76.174 | 1.00 | 60.42 | C |
| ATOM | 13510 | CG | GLN | D | 86 | 56.455 | 65.786 | 74.922 | 1.00 | 62.18 | C |
| ATOM | 13513 | CD | GLN | D | 86 | 57.886 | 66.248 | 75.220 | 1.00 | 64.34 | C |
| ATOM | 13514 | OE1 | GLN | D | 86 | 58.158 | 66.968 | 76.214 | 1.00 | 62.43 | O |
| ATOM | 13515 | NE2 | GLN | D | 86 | 58.800 | 65.806 | 74.377 | 1.00 | 62.47 | N |
| ATOM | 13518 | C | GLN | D | 86 | 55.487 | 64.077 | 78.249 | 1.00 | 56.98 | C |
| ATOM | 13519 | O | GLN | D | 86 | 55.039 | 62.988 | 78.450 | 1.00 | 53.19 | O |
| ATOM | 13520 | N | SER | D | 87 | 55.323 | 65.069 | 79.089 | 1.00 | 59.59 | N |
| ATOM | 13522 | CA | SER | D | 87 | 54.745 | 64.812 | 80.381 | 1.00 | 59.57 | C |
| ATOM | 13524 | CB | SER | D | 87 | 55.036 | 65.975 | 81.289 | 1.00 | 63.01 | C |
| ATOM | 13527 | OG | SER | D | 87 | 54.389 | 67.041 | 80.689 | 1.00 | 67.52 | O |
| ATOM | 13529 | C | SER | D | 87 | 53.258 | 64.544 | 80.272 | 1.00 | 57.33 | C |
| ATOM | 13530 | O | SER | D | 87 | 52.767 | 63.721 | 81.021 | 1.00 | 55.78 | O |
| ATOM | 13531 | N | ASN | D | 88 | 52.563 | 65.209 | 79.329 | 1.00 | 58.66 | N |
| ATOM | 13533 | CA | ASN | D | 88 | 51.256 | 64.748 | 78.718 | 1.00 | 56.25 | C |
| ATOM | 13535 | CB | ASN | D | 88 | 51.180 | 65.170 | 77.247 | 1.00 | 57.45 | C |
| ATOM | 13538 | CG | ASN | D | 88 | 50.876 | 66.580 | 77.067 | 1.00 | 62.84 | C |
| ATOM | 13539 | OD1 | ASN | D | 88 | 50.883 | 67.306 | 78.041 | 1.00 | 74.09 | O |
| ATOM | 13540 | ND2 | ASN | D | 88 | 50.533 | 67.012 | 75.831 | 1.00 | 63.95 | N |
| ATOM | 13543 | C | ASN | D | 88 | 51.038 | 63.237 | 78.581 | 1.00 | 52.90 | C |
| ATOM | 13544 | O | ASN | D | 88 | 49.916 | 62.814 | 78.417 | 1.00 | 52.43 | O |
| ATOM | 13545 | N | ASP | D | 89 | 52.092 | 62.440 | 78.501 | 1.00 | 51.41 | N |
| ATOM | 13547 | CA | ASP | D | 89 | 51.949 | 61.018 | 78.388 | 1.00 | 49.06 | C |
| ATOM | 13549 | CB | ASP | D | 89 | 52.926 | 60.536 | 77.355 | 1.00 | 49.14 | C |
| ATOM | 13552 | CG | ASP | D | 89 | 52.744 | 61.234 | 76.046 | 1.00 | 51.56 | C |
| ATOM | 13553 | OD1 | ASP | D | 89 | 51.627 | 61.127 | 75.481 | 1.00 | 51.32 | O |
| ATOM | 13554 | OD2 | ASP | D | 89 | 53.661 | 61.899 | 75.486 | 1.00 | 54.26 | O |
| ATOM | 13555 | C | ASP | D | 89 | 52.113 | 60.193 | 79.688 | 1.00 | 49.01 | C |
| ATOM | 13556 | O | ASP | D | 89 | 52.119 | 58.945 | 79.650 | 1.00 | 47.85 | O |
| ATOM | 13557 | N | THR | D | 90 | 52.206 | 60.851 | 80.847 | 1.00 | 51.07 | N |
| ATOM | 13559 | CA | THR | D | 90 | 52.121 | 60.112 | 82.141 | 1.00 | 49.85 | C |
| ATOM | 13561 | CB | THR | D | 90 | 52.273 | 61.030 | 83.288 | 1.00 | 51.99 | C |
| ATOM | 13563 | OG1 | THR | D | 90 | 53.571 | 61.636 | 83.183 | 1.00 | 57.36 | O |
| ATOM | 13565 | CG2 | THR | D | 90 | 52.303 | 60.281 | 84.588 | 1.00 | 51.56 | C |
| ATOM | 13569 | C | THR | D | 90 | 50.773 | 59.577 | 82.214 | 1.00 | 47.11 | C |
| ATOM | 13570 | O | THR | D | 90 | 49.808 | 60.323 | 81.988 | 1.00 | 47.64 | O |
| ATOM | 13571 | N | ALA | D | 91 | 50.695 | 58.289 | 82.502 | 1.00 | 45.03 | N |
| ATOM | 13573 | CA | ALA | D | 91 | 49.405 | 57.551 | 82.488 | 1.00 | 42.38 | C |
| ATOM | 13575 | CB | ALA | D | 91 | 48.762 | 57.590 | 81.130 | 1.00 | 41.09 | C |
| ATOM | 13579 | C | ALA | D | 91 | 49.640 | 56.147 | 82.851 | 1.00 | 40.25 | C |
| ATOM | 13580 | O | ALA | D | 91 | 50.767 | 55.723 | 82.869 | 1.00 | 41.74 | O |
| ATOM | 13581 | N | ILE | D | 92 | 48.576 | 55.431 | 83.165 | 1.00 | 38.65 | N |
| ATOM | 13583 | CA | ILE | D | 92 | 48.616 | 53.982 | 83.189 | 1.00 | 36.52 | C |
| ATOM | 13585 | CB | ILE | D | 92 | 47.692 | 53.500 | 84.265 | 1.00 | 37.41 | C |
| ATOM | 13587 | CG1 | ILE | D | 92 | 48.318 | 53.873 | 85.587 | 1.00 | 39.88 | C |
| ATOM | 13590 | CD1 | ILE | D | 92 | 47.426 | 54.065 | 86.604 | 1.00 | 43.89 | C |
| ATOM | 13594 | CG2 | ILE | D | 92 | 47.526 | 52.047 | 84.213 | 1.00 | 36.13 | C |
| ATOM | 13598 | C | ILE | D | 92 | 48.310 | 53.461 | 81.803 | 1.00 | 33.73 | C |
| ATOM | 13599 | O | ILE | D | 92 | 47.290 | 53.775 | 81.214 | 1.00 | 32.00 | O |
| ATOM | 13600 | N | TYR | D | 93 | 49.248 | 52.737 | 81.244 | 1.00 | 33.28 | N |
| ATOM | 13602 | CA | TYR | D | 93 | 49.012 | 52.153 | 79.948 | 1.00 | 32.94 | C |
| ATOM | 13604 | CB | TYR | D | 93 | 50.261 | 52.260 | 79.101 | 1.00 | 32.51 | C |
| ATOM | 13607 | CG | TYR | D | 93 | 50.564 | 53.690 | 78.742 | 1.00 | 35.57 | C |
| ATOM | 13608 | CD1 | TYR | D | 93 | 50.996 | 54.606 | 79.683 | 1.00 | 38.55 | C |
| ATOM | 13610 | CE1 | TYR | D | 93 | 51.271 | 55.942 | 79.320 | 1.00 | 40.44 | C |
| ATOM | 13612 | CZ | TYR | D | 93 | 51.095 | 56.353 | 78.008 | 1.00 | 38.75 | C |
| ATOM | 13613 | OH | TYR | D | 93 | 51.301 | 57.632 | 77.620 | 1.00 | 36.83 | O |
| ATOM | 13615 | CE2 | TYR | D | 93 | 50.676 | 55.468 | 77.070 | 1.00 | 37.22 | C |
| ATOM | 13617 | CD2 | TYR | D | 93 | 50.386 | 54.151 | 77.450 | 1.00 | 37.91 | C |
| ATOM | 13619 | C | TYR | D | 93 | 48.527 | 50.698 | 80.099 | 1.00 | 32.47 | C |
| ATOM | 13620 | O | TYR | D | 93 | 49.158 | 49.906 | 80.771 | 1.00 | 33.93 | O |
| ATOM | 13621 | N | TYR | D | 94 | 47.412 | 50.374 | 79.461 | 1.00 | 31.81 | N |
| ATOM | 13623 | CA | TYR | D | 94 | 46.790 | 49.071 | 79.509 | 1.00 | 31.89 | C |
| ATOM | 13625 | CB | TYR | D | 94 | 45.316 | 49.180 | 79.927 | 1.00 | 31.43 | C |
| ATOM | 13628 | CG | TYR | D | 94 | 45.044 | 49.623 | 81.317 | 1.00 | 34.26 | C |
| ATOM | 13629 | CD1 | TYR | D | 94 | 44.722 | 50.933 | 81.611 | 1.00 | 34.32 | C |
| ATOM | 13631 | CE1 | TYR | D | 94 | 44.471 | 51.333 | 82.955 | 1.00 | 37.87 | C |
| ATOM | 13633 | CZ | TYR | D | 94 | 44.541 | 50.406 | 83.981 | 1.00 | 40.08 | C |
| ATOM | 13634 | OH | TYR | D | 94 | 44.302 | 50.719 | 85.322 | 1.00 | 44.06 | O |
| ATOM | 13636 | CE2 | TYR | D | 94 | 44.840 | 49.120 | 83.681 | 1.00 | 39.37 | C |
| ATOM | 13638 | CD2 | TYR | D | 94 | 45.065 | 48.720 | 82.362 | 1.00 | 37.60 | C |
| ATOM | 13640 | C | TYR | D | 94 | 46.766 | 48.458 | 78.114 | 1.00 | 31.19 | C |
| ATOM | 13641 | O | TYR | D | 94 | 46.595 | 49.162 | 77.134 | 1.00 | 28.84 | O |
| ATOM | 13642 | N | CYS | D | 95 | 46.803 | 47.114 | 78.094 | 1.00 | 32.31 | N |
| ATOM | 13644 | CA | CYS | D | 95 | 46.343 | 46.326 | 76.978 | 1.00 | 31.63 | C |
| ATOM | 13646 | CB | CYS | D | 95 | 47.407 | 45.367 | 76.555 | 1.00 | 32.86 | C |
| ATOM | 13649 | SG | CYS | D | 95 | 47.837 | 44.087 | 77.682 | 1.00 | 36.32 | S |
| ATOM | 13650 | C | CYS | D | 95 | 45.043 | 45.595 | 77.229 | 1.00 | 31.81 | C |
| ATOM | 13651 | O | CYS | D | 95 | 44.543 | 45.489 | 78.338 | 1.00 | 32.84 | O |
| ATOM | 13652 | N | ALA | D | 96 | 44.415 | 45.138 | 76.170 | 1.00 | 31.66 | N |
| ATOM | 13654 | CA | ALA | D | 96 | 43.065 | 44.613 | 76.333 | 1.00 | 31.98 | C |
| ATOM | 13656 | CB | ALA | D | 96 | 42.067 | 45.738 | 76.560 | 1.00 | 31.73 | C |
| ATOM | 13660 | C | ALA | D | 96 | 42.610 | 43.789 | 75.189 | 1.00 | 31.15 | C |
| ATOM | 13661 | O | ALA | D | 96 | 43.109 | 43.916 | 74.113 | 1.00 | 30.28 | O |
| ATOM | 13662 | N | ARG | D | 97 | 41.644 | 42.929 | 75.459 | 1.00 | 31.57 | N |
| ATOM | 13664 | CA | ARG | D | 97 | 41.056 | 42.132 | 74.424 | 1.00 | 31.04 | C |
| ATOM | 13666 | CB | ARG | D | 97 | 41.403 | 40.657 | 74.529 | 1.00 | 31.65 | C |
| ATOM | 13669 | CG | ARG | D | 97 | 41.364 | 39.961 | 73.252 | 1.00 | 30.26 | C |
| ATOM | 13672 | CD | ARG | D | 97 | 41.106 | 38.522 | 73.380 | 1.00 | 33.63 | C |
| ATOM | 13675 | NE | ARG | D | 97 | 39.673 | 38.270 | 73.544 | 1.00 | 35.37 | N |
| ATOM | 13677 | CZ | ARG | D | 97 | 39.119 | 37.052 | 73.723 | 1.00 | 37.21 | C |
| ATOM | 13678 | NH1 | ARG | D | 97 | 39.855 | 35.971 | 73.753 | 1.00 | 39.36 | N |
| ATOM | 13681 | NH2 | ARG | D | 97 | 37.797 | 36.905 | 73.786 | 1.00 | 38.08 | N |
| ATOM | 13684 | C | ARG | D | 97 | 39.632 | 42.302 | 74.578 | 1.00 | 30.96 | C |
| ATOM | 13685 | O | ARG | D | 97 | 39.130 | 42.243 | 75.686 | 1.00 | 31.79 | O |
| ATOM | 13686 | N | ALA | D | 98 | 38.981 | 42.482 | 73.441 | 1.00 | 30.39 | N |
| ATOM | 13688 | CA | ALA | D | 98 | 37.540 | 42.481 | 73.374 | 1.00 | 30.35 | C |
| ATOM | 13690 | CB | ALA | D | 98 | 37.141 | 42.731 | 71.958 | 1.00 | 30.65 | C |
| ATOM | 13694 | C | ALA | D | 98 | 36.991 | 41.140 | 73.769 | 1.00 | 31.90 | C |
| ATOM | 13695 | O | ALA | D | 98 | 37.675 | 40.141 | 73.670 | 1.00 | 31.68 | O |
| ATOM | 13696 | N | LEU | D | 99 | 35.719 | 41.124 | 74.134 | 1.00 | 33.49 | N |
| ATOM | 13698 | CA | LEU | D | 99 | 34.995 | 39.875 | 74.405 | 1.00 | 35.36 | C |
| ATOM | 13700 | CB | LEU | D | 99 | 33.719 | 40.195 | 75.166 | 1.00 | 36.53 | C |
| ATOM | 13703 | CG | LEU | D | 99 | 32.855 | 39.064 | 75.632 | 1.00 | 37.57 | C |
| ATOM | 13705 | CD1 | LEU | D | 99 | 33.465 | 38.470 | 76.786 | 1.00 | 39.95 | C |
| ATOM | 13709 | CD2 | LEU | D | 99 | 31.538 | 39.533 | 75.980 | 1.00 | 38.23 | C |
| ATOM | 13713 | C | LEU | D | 99 | 34.636 | 39.123 | 73.129 | 1.00 | 34.97 | C |
| ATOM | 13714 | O | LEU | D | 99 | 34.376 | 37.955 | 73.165 | 1.00 | 38.11 | O |
| ATOM | 13715 | N | THR | D | 100 | 34.604 | 39.801 | 72.007 | 1.00 | 33.14 | N |
| ATOM | 13717 | CA | THR | D | 100 | 34.004 | 39.285 | 70.772 | 1.00 | 32.28 | C |
| ATOM | 13719 | CB | THR | D | 100 | 32.617 | 39.874 | 70.631 | 1.00 | 33.08 | C |
| ATOM | 13721 | OG1 | THR | D | 100 | 31.693 | 39.072 | 71.380 | 1.00 | 32.58 | O |
| ATOM | 13723 | CG2 | THR | D | 100 | 32.098 | 39.948 | 69.118 | 1.00 | 37.21 | C |
| ATOM | 13727 | C | THR | D | 100 | 34.873 | 39.702 | 69.604 | 1.00 | 31.13 | C |
| ATOM | 13728 | O | THR | D | 100 | 35.471 | 40.785 | 69.620 | 1.00 | 30.37 | O |
| ATOM | 13729 | N | TYR | D | 101 | 34.993 | 38.833 | 68.611 | 1.00 | 31.37 | N |
| ATOM | 13731 | CA | TYR | D | 101 | 35.703 | 39.160 | 67.419 | 1.00 | 30.84 | C |
| ATOM | 13733 | CB | TYR | D | 101 | 35.437 | 38.137 | 66.314 | 1.00 | 32.08 | C |
| ATOM | 13736 | CG | TYR | D | 101 | 35.629 | 36.729 | 66.805 | 1.00 | 34.25 | C |
| ATOM | 13737 | CD1 | TYR | D | 101 | 34.583 | 35.941 | 67.097 | 1.00 | 38.88 | C |
| ATOM | 13739 | CE1 | TYR | D | 101 | 34.750 | 34.707 | 67.562 | 1.00 | 39.52 | C |
| ATOM | 13741 | CZ | TYR | D | 101 | 35.946 | 34.269 | 67.763 | 1.00 | 38.52 | C |
| ATOM | 13742 | OH | TYR | D | 101 | 36.095 | 33.043 | 68.238 | 1.00 | 44.46 | O |
| ATOM | 13744 | CE2 | TYR | D | 101 | 36.986 | 34.992 | 67.497 | 1.00 | 36.82 | C |
| ATOM | 13746 | CD2 | TYR | D | 101 | 36.835 | 36.231 | 67.048 | 1.00 | 35.53 | C |
| ATOM | 13748 | C | TYR | D | 101 | 35.277 | 40.529 | 66.932 | 1.00 | 30.86 | C |
| ATOM | 13749 | O | TYR | D | 101 | 34.084 | 40.893 | 66.955 | 1.00 | 33.11 | O |
| ATOM | 13750 | N | TYR | D | 102 | 36.280 | 41.279 | 66.517 | 1.00 | 29.81 | N |
| ATOM | 13752 | CA | TYR | D | 102 | 36.191 | 42.562 | 65.853 | 1.00 | 29.45 | C |
| ATOM | 13754 | CB | TYR | D | 102 | 35.565 | 42.386 | 64.481 | 1.00 | 30.72 | C |
| ATOM | 13757 | CG | TYR | D | 102 | 36.154 | 41.295 | 63.698 | 1.00 | 28.52 | C |
| ATOM | 13758 | CD1 | TYR | D | 102 | 35.328 | 40.351 | 63.085 | 1.00 | 32.83 | C |
| ATOM | 13760 | CE1 | TYR | D | 102 | 35.834 | 39.306 | 62.287 | 1.00 | 32.70 | C |
| ATOM | 13762 | CZ | TYR | D | 102 | 37.178 | 39.172 | 62.157 | 1.00 | 29.94 | C |
| ATOM | 13763 | OH | TYR | D | 102 | 37.581 | 38.164 | 61.424 | 1.00 | 30.00 | O |
| ATOM | 13765 | CE2 | TYR | D | 102 | 38.019 | 40.076 | 62.744 | 1.00 | 29.21 | C |
| ATOM | 13767 | CD2 | TYR | D | 102 | 37.461 | 41.196 | 63.511 | 1.00 | 26.98 | C |
| ATOM | 13769 | C | TYR | D | 102 | 35.564 | 43.702 | 66.606 | 1.00 | 29.34 | C |
| ATOM | 13770 | O | TYR | D | 102 | 35.211 | 44.753 | 65.993 | 1.00 | 31.06 | O |
| ATOM | 13771 | N | ASP | D | 103 | 35.453 | 43.560 | 67.911 | 1.00 | 29.51 | N |
| ATOM | 13773 | CA | ASP | D | 103 | 34.621 | 44.510 | 68.693 | 1.00 | 31.57 | C |
| ATOM | 13775 | CB | ASP | D | 103 | 33.523 | 43.743 | 69.392 | 1.00 | 32.31 | C |
| ATOM | 13778 | CG | ASP | D | 103 | 32.231 | 44.541 | 69.603 | 1.00 | 35.50 | C |
| ATOM | 13779 | OD1 | ASP | D | 103 | 32.229 | 45.811 | 69.699 | 1.00 | 37.86 | O |
| ATOM | 13780 | OD2 | ASP | D | 103 | 31.147 | 43.932 | 69.760 | 1.00 | 33.03 | O |
| ATOM | 13781 | C | ASP | D | 103 | 35.493 | 45.285 | 69.688 | 1.00 | 31.31 | C |
| ATOM | 13782 | O | ASP | D | 103 | 36.726 | 45.130 | 69.654 | 1.00 | 30.80 | O |
| ATOM | 13783 | N | TYR | D | 104 | 34.867 | 46.062 | 70.572 | 1.00 | 32.47 | N |
| ATOM | 13785 | CA | TYR | D | 104 | 35.564 | 47.042 | 71.419 | 1.00 | 33.02 | C |
| ATOM | 13787 | CB | TYR | D | 104 | 35.387 | 48.429 | 70.847 | 1.00 | 32.52 | C |
| ATOM | 13790 | CG | TYR | D | 104 | 35.855 | 48.529 | 69.452 | 1.00 | 32.49 | C |
| ATOM | 13791 | CD1 | TYR | D | 104 | 35.051 | 48.168 | 68.386 | 1.00 | 34.38 | C |
| ATOM | 13793 | CE1 | TYR | D | 104 | 35.511 | 48.235 | 67.060 | 1.00 | 36.47 | C |
| ATOM | 13795 | CZ | TYR | D | 104 | 36.784 | 48.661 | 66.851 | 1.00 | 40.26 | C |
| ATOM | 13796 | OH | TYR | D | 104 | 37.335 | 48.820 | 65.555 | 1.00 | 42.04 | O |
| ATOM | 13798 | CE2 | TYR | D | 104 | 37.581 | 48.984 | 67.959 | 1.00 | 36.79 | C |
| ATOM | 13800 | CD2 | TYR | D | 104 | 37.121 | 48.927 | 69.194 | 1.00 | 32.46 | C |
| ATOM | 13802 | C | TYR | D | 104 | 35.042 | 47.079 | 72.872 | 1.00 | 35.19 | C |
| ATOM | 13803 | O | TYR | D | 104 | 35.119 | 48.144 | 73.559 | 1.00 | 36.99 | O |
| ATOM | 13804 | N | GLU | D | 105 | 34.624 | 45.896 | 73.331 | 1.00 | 35.25 | N |
| ATOM | 13806 | CA | GLU | D | 105 | 33.892 | 45.637 | 74.609 | 1.00 | 37.05 | C |
| ATOM | 13808 | CB | GLU | D | 105 | 32.653 | 44.759 | 74.311 | 1.00 | 38.39 | C |
| ATOM | 13811 | CG | GLU | D | 105 | 33.162 | 43.726 | 73.210 | 1.00 | 39.20 | C |
| ATOM | 13814 | CD | GLU | D | 105 | 32.119 | 42.797 | 72.800 | 1.00 | 43.24 | C |
| ATOM | 13815 | OE1 | GLU | D | 105 | 30.967 | 43.102 | 73.166 | 1.00 | 50.60 | O |
| ATOM | 13816 | OE2 | GLU | D | 105 | 32.424 | 41.815 | 72.145 | 1.00 | 41.17 | O |
| ATOM | 13817 | C | GLU | D | 105 | 34.955 | 44.885 | 75.449 | 1.00 | 34.79 | C |
| ATOM | 13818 | O | GLU | D | 105 | 35.096 | 43.674 | 75.450 | 1.00 | 33.21 | O |
| ATOM | 13819 | N | PHE | D | 106 | 35.794 | 45.663 | 76.079 | 1.00 | 34.74 | N |
| ATOM | 13821 | CA | PHE | D | 106 | 36.971 | 45.106 | 76.719 | 1.00 | 33.56 | C |
| ATOM | 13823 | CB | PHE | D | 106 | 37.941 | 46.202 | 76.907 | 1.00 | 32.11 | C |
| ATOM | 13826 | CG | PHE | D | 106 | 38.218 | 46.917 | 75.658 | 1.00 | 32.00 | C |
| ATOM | 13827 | CD1 | PHE | D | 106 | 38.095 | 48.287 | 75.563 | 1.00 | 36.38 | C |
| ATOM | 13829 | CE1 | PHE | D | 106 | 38.440 | 48.935 | 74.393 | 1.00 | 34.66 | C |
| ATOM | 13831 | CZ | PHE | D | 106 | 38.862 | 48.191 | 73.304 | 1.00 | 30.31 | C |
| ATOM | 13833 | CE2 | PHE | D | 106 | 38.918 | 46.865 | 73.389 | 1.00 | 29.09 | C |
| ATOM | 13835 | CD2 | PHE | D | 106 | 38.605 | 46.234 | 74.565 | 1.00 | 30.59 | C |
| ATOM | 13837 | C | PHE | D | 106 | 36.657 | 44.375 | 78.016 | 1.00 | 34.43 | C |
| ATOM | 13838 | O | PHE | D | 106 | 36.441 | 44.995 | 79.034 | 1.00 | 36.37 | O |
| ATOM | 13839 | N | ALA | D | 107 | 36.649 | 43.050 | 77.934 | 1.00 | 33.57 | N |
| ATOM | 13841 | CA | ALA | D | 107 | 36.320 | 42.181 | 79.035 | 1.00 | 35.28 | C |
| ATOM | 13843 | CB | ALA | D | 107 | 35.614 | 40.888 | 78.539 | 1.00 | 35.86 | C |
| ATOM | 13847 | C | ALA | D | 107 | 37.554 | 41.780 | 79.759 | 1.00 | 35.97 | C |
| ATOM | 13848 | O | ALA | D | 107 | 37.409 | 41.351 | 80.903 | 1.00 | 38.66 | O |
| ATOM | 13849 | N | TYR | D | 108 | 38.716 | 41.898 | 79.101 | 1.00 | 33.76 | N |
| ATOM | 13851 | CA | TYR | D | 108 | 39.993 | 41.378 | 79.567 | 1.00 | 34.96 | C |
| ATOM | 13853 | CB | TYR | D | 108 | 40.460 | 40.163 | 78.709 | 1.00 | 34.47 | C |
| ATOM | 13856 | CG | TYR | D | 108 | 39.440 | 39.099 | 78.601 | 1.00 | 35.04 | C |
| ATOM | 13857 | CD1 | TYR | D | 108 | 38.726 | 38.850 | 77.407 | 1.00 | 35.31 | C |
| ATOM | 13859 | CE1 | TYR | D | 108 | 37.743 | 37.862 | 77.365 | 1.00 | 35.79 | C |
| ATOM | 13861 | CZ | TYR | D | 108 | 37.474 | 37.182 | 78.535 | 1.00 | 38.03 | C |
| ATOM | 13862 | OH | TYR | D | 108 | 36.533 | 36.206 | 78.654 | 1.00 | 44.30 | O |
| ATOM | 13864 | CE2 | TYR | D | 108 | 38.152 | 37.435 | 79.680 | 1.00 | 38.12 | C |
| ATOM | 13866 | CD2 | TYR | D | 108 | 39.139 | 38.355 | 79.697 | 1.00 | 37.41 | C |
| ATOM | 13868 | C | TYR | D | 108 | 41.038 | 42.466 | 79.377 | 1.00 | 34.37 | C |
| ATOM | 13869 | O | TYR | D | 108 | 41.156 | 42.969 | 78.276 | 1.00 | 33.58 | O |
| ATOM | 13870 | N | TRP | D | 109 | 41.817 | 42.778 | 80.431 | 1.00 | 35.51 | N |
| ATOM | 13872 | CA | TRP | D | 109 | 42.755 | 43.898 | 80.499 | 1.00 | 33.04 | C |
| ATOM | 13874 | CB | TRP | D | 109 | 42.188 | 45.019 | 81.373 | 1.00 | 33.12 | C |
| ATOM | 13877 | CG | TRP | D | 109 | 40.965 | 45.716 | 80.852 | 1.00 | 33.03 | C |
| ATOM | 13878 | CD1 | TRP | D | 109 | 39.735 | 45.182 | 80.686 | 1.00 | 35.04 | C |
| ATOM | 13880 | NE1 | TRP | D | 109 | 38.877 | 46.105 | 80.136 | 1.00 | 35.58 | N |
| ATOM | 13882 | CE2 | TRP | D | 109 | 39.559 | 47.266 | 79.932 | 1.00 | 33.94 | C |
| ATOM | 13883 | CD2 | TRP | D | 109 | 40.878 | 47.049 | 80.343 | 1.00 | 33.28 | C |
| ATOM | 13884 | CE3 | TRP | D | 109 | 41.789 | 48.075 | 80.166 | 1.00 | 33.80 | C |
| ATOM | 13886 | CZ3 | TRP | D | 109 | 41.349 | 49.277 | 79.611 | 1.00 | 33.42 | C |
| ATOM | 13888 | CH2 | TRP | D | 109 | 40.051 | 49.447 | 79.228 | 1.00 | 33.69 | C |
| ATOM | 13890 | CZ2 | TRP | D | 109 | 39.129 | 48.465 | 79.401 | 1.00 | 33.31 | C |
| ATOM | 13892 | C | TRP | D | 109 | 43.970 | 43.405 | 81.176 | 1.00 | 34.87 | C |
| ATOM | 13893 | O | TRP | D | 109 | 43.919 | 42.614 | 82.081 | 1.00 | 37.35 | O |
| ATOM | 13894 | N | GLY | D | 110 | 45.108 | 43.904 | 80.769 | 1.00 | 35.09 | N |
| ATOM | 13896 | CA | GLY | D | 110 | 46.304 | 43.773 | 81.543 | 1.00 | 35.48 | C |
| ATOM | 13899 | C | GLY | D | 110 | 46.167 | 44.509 | 82.858 | 1.00 | 36.71 | C |
| ATOM | 13900 | O | GLY | D | 110 | 45.170 | 45.206 | 83.165 | 1.00 | 35.65 | O |
| ATOM | 13901 | N | GLN | D | 111 | 47.194 | 44.292 | 83.674 | 1.00 | 38.41 | N |
| ATOM | 13903 | CA | GLN | D | 111 | 47.290 | 44.909 | 84.986 | 1.00 | 39.88 | C |
| ATOM | 13905 | CB | GLN | D | 111 | 48.241 | 44.104 | 85.886 | 1.00 | 42.53 | C |
| ATOM | 13908 | CG | GLN | D | 111 | 49.725 | 44.447 | 85.776 | 1.00 | 42.86 | C |
| ATOM | 13911 | CD | GLN | D | 111 | 50.463 | 43.616 | 84.777 | 1.00 | 41.89 | C |
| ATOM | 13912 | OE1 | GLN | D | 111 | 51.637 | 43.438 | 84.917 | 1.00 | 44.32 | O |
| ATOM | 13913 | NE2 | GLN | D | 111 | 49.790 | 43.179 | 83.713 | 1.00 | 41.71 | N |
| ATOM | 13916 | C | GLN | D | 111 | 47.694 | 46.381 | 84.902 | 1.00 | 38.77 | C |
| ATOM | 13917 | O | GLN | D | 111 | 47.649 | 47.095 | 85.877 | 1.00 | 40.58 | O |
| ATOM | 13918 | N | GLY | D | 112 | 48.078 | 46.845 | 83.738 | 1.00 | 36.79 | N |
| ATOM | 13920 | CA | GLY | D | 112 | 48.429 | 48.224 | 83.606 | 1.00 | 37.21 | C |
| ATOM | 13923 | C | GLY | D | 112 | 49.899 | 48.474 | 83.858 | 1.00 | 39.24 | C |
| ATOM | 13924 | O | GLY | D | 112 | 50.520 | 47.788 | 84.674 | 1.00 | 42.52 | O |
| ATOM | 13925 | N | THR | D | 113 | 50.463 | 49.436 | 83.139 | 1.00 | 38.68 | N |
| ATOM | 13927 | CA | THR | D | 113 | 51.785 | 49.879 | 83.417 | 1.00 | 41.03 | C |
| ATOM | 13929 | CB | THR | D | 113 | 52.612 | 49.719 | 82.180 | 1.00 | 40.97 | C |
| ATOM | 13931 | OG1 | THR | D | 113 | 52.785 | 48.325 | 81.891 | 1.00 | 40.85 | O |
| ATOM | 13933 | CG2 | THR | D | 113 | 54.036 | 50.254 | 82.416 | 1.00 | 42.53 | C |
| ATOM | 13937 | C | THR | D | 113 | 51.750 | 51.353 | 83.870 | 1.00 | 42.14 | C |
| ATOM | 13938 | O | THR | D | 113 | 51.250 | 52.198 | 83.134 | 1.00 | 40.31 | O |
| ATOM | 13939 | N | LEU | D | 114 | 52.270 | 51.637 | 85.076 | 1.00 | 44.50 | N |
| ATOM | 13941 | CA | LEU | D | 114 | 52.295 | 52.984 | 85.606 | 1.00 | 45.15 | C |
| ATOM | 13943 | CB | LEU | D | 114 | 52.560 | 52.927 | 87.087 | 1.00 | 47.24 | C |
| ATOM | 13946 | CG | LEU | D | 114 | 52.578 | 54.312 | 87.753 | 1.00 | 48.79 | C |
| ATOM | 13948 | CD1 | LEU | D | 114 | 51.232 | 54.921 | 87.733 | 1.00 | 49.87 | C |
| ATOM | 13952 | CD2 | LEU | D | 114 | 53.087 | 54.296 | 89.213 | 1.00 | 51.70 | C |
| ATOM | 13956 | C | LEU | D | 114 | 53.444 | 53.717 | 84.956 | 1.00 | 46.43 | C |
| ATOM | 13957 | O | LEU | D | 114 | 54.584 | 53.494 | 85.359 | 1.00 | 48.45 | O |
| ATOM | 13958 | N | VAL | D | 115 | 53.174 | 54.617 | 84.008 | 1.00 | 44.80 | N |
| ATOM | 13960 | CA | VAL | D | 115 | 54.248 | 55.285 | 83.274 | 1.00 | 45.32 | C |
| ATOM | 13962 | CB | VAL | D | 115 | 53.999 | 55.303 | 81.781 | 1.00 | 43.97 | C |
| ATOM | 13964 | CG1 | VAL | D | 115 | 55.038 | 56.092 | 81.076 | 1.00 | 45.37 | C |
| ATOM | 13968 | CG2 | VAL | D | 115 | 54.011 | 53.915 | 81.240 | 1.00 | 45.06 | C |
| ATOM | 13972 | C | VAL | D | 115 | 54.332 | 56.717 | 83.635 | 1.00 | 46.21 | C |
| ATOM | 13973 | O | VAL | D | 115 | 53.342 | 57.421 | 83.507 | 1.00 | 46.25 | O |
| ATOM | 13974 | N | THR | D | 116 | 55.549 | 57.149 | 83.931 | 1.00 | 47.39 | N |
| ATOM | 13976 | CA | THR | D | 116 | 55.876 | 58.442 | 84.504 | 1.00 | 49.19 | C |
| ATOM | 13978 | CB | THR | D | 116 | 56.559 | 58.139 | 85.826 | 1.00 | 51.28 | C |
| ATOM | 13980 | OG1 | THR | D | 116 | 55.851 | 57.040 | 86.446 | 1.00 | 49.66 | O |
| ATOM | 13982 | CG2 | THR | D | 116 | 56.561 | 59.345 | 86.814 | 1.00 | 52.90 | C |
| ATOM | 13986 | C | THR | D | 116 | 56.870 | 59.154 | 83.611 | 1.00 | 50.16 | C |
| ATOM | 13987 | O | THR | D | 116 | 57.880 | 58.600 | 83.330 | 1.00 | 50.46 | O |
| ATOM | 13988 | N | VAL | D | 117 | 56.626 | 60.382 | 83.209 | 1.00 | 50.68 | N |
| ATOM | 13990 | CA | VAL | D | 117 | 57.511 | 61.021 | 82.292 | 1.00 | 52.90 | C |
| ATOM | 13992 | CB | VAL | D | 117 | 56.786 | 61.424 | 81.067 | 1.00 | 52.02 | C |
| ATOM | 13994 | CG1 | VAL | D | 117 | 57.669 | 62.056 | 80.124 | 1.00 | 54.34 | C |
| ATOM | 13998 | CG2 | VAL | D | 117 | 56.235 | 60.219 | 80.389 | 1.00 | 51.45 | C |
| ATOM | 14002 | C | VAL | D | 117 | 58.169 | 62.215 | 82.941 | 1.00 | 57.15 | C |
| ATOM | 14003 | O | VAL | D | 117 | 57.575 | 63.264 | 83.139 | 1.00 | 59.15 | O |
| ATOM | 14004 | N | SER | D | 118 | 59.443 | 62.064 | 83.257 | 1.00 | 59.82 | N |
| ATOM | 14006 | CA | SER | D | 118 | 60.162 | 63.096 | 83.950 | 1.00 | 63.47 | C |
| ATOM | 14008 | CB | SER | D | 118 | 59.855 | 62.996 | 85.452 | 1.00 | 63.79 | C |
| ATOM | 14011 | OG | SER | D | 118 | 60.736 | 63.764 | 86.268 | 1.00 | 66.67 | O |
| ATOM | 14013 | C | SER | D | 118 | 61.614 | 62.834 | 83.640 | 1.00 | 66.29 | C |
| ATOM | 14014 | O | SER | D | 118 | 62.005 | 61.685 | 83.390 | 1.00 | 64.91 | O |
| ATOM | 14015 | N | ALA | D | 119 | 62.406 | 63.899 | 83.602 | 1.00 | 54.31 | N |
| ATOM | 14017 | CA | ALA | D | 119 | 63.822 | 63.741 | 83.347 | 1.00 | 55.75 | C |
| ATOM | 14019 | CB | ALA | D | 119 | 64.349 | 64.894 | 82.555 | 1.00 | 57.76 | C |
| ATOM | 14023 | C | ALA | D | 119 | 64.549 | 63.610 | 84.672 | 1.00 | 55.45 | C |
| ATOM | 14024 | O | ALA | D | 119 | 65.775 | 63.529 | 84.729 | 1.00 | 56.97 | O |
| ATOM | 14025 | N | ALA | D | 120 | 63.807 | 63.582 | 85.753 | 1.00 | 53.85 | N |
| ATOM | 14027 | CA | ALA | D | 120 | 64.453 | 63.464 | 87.038 | 1.00 | 53.86 | C |
| ATOM | 14029 | CB | ALA | D | 120 | 63.469 | 63.824 | 88.155 | 1.00 | 53.59 | C |
| ATOM | 14033 | C | ALA | D | 120 | 64.930 | 62.055 | 87.236 | 1.00 | 53.49 | C |
| ATOM | 14034 | O | ALA | D | 120 | 64.510 | 61.091 | 86.561 | 1.00 | 52.47 | O |
| ATOM | 14035 | N | SER | D | 121 | 65.770 | 61.966 | 88.241 | 1.00 | 54.49 | N |
| ATOM | 14037 | CA | SER | D | 121 | 66.445 | 60.753 | 88.607 | 1.00 | 55.63 | C |
| ATOM | 14039 | CB | SER | D | 121 | 67.839 | 61.096 | 89.074 | 1.00 | 58.28 | C |
| ATOM | 14042 | OG | SER | D | 121 | 67.715 | 61.672 | 90.340 | 1.00 | 57.66 | O |
| ATOM | 14044 | C | SER | D | 121 | 65.723 | 60.084 | 89.756 | 1.00 | 54.50 | C |
| ATOM | 14045 | O | SER | D | 121 | 65.234 | 60.737 | 90.667 | 1.00 | 54.17 | O |
| ATOM | 14046 | N | THR | D | 122 | 65.671 | 58.761 | 89.676 | 1.00 | 54.53 | N |
| ATOM | 14048 | CA | THR | D | 122 | 65.177 | 57.902 | 90.720 | 1.00 | 53.23 | C |
| ATOM | 14050 | CB | THR | D | 122 | 65.387 | 56.504 | 90.241 | 1.00 | 54.78 | C |
| ATOM | 14052 | OG1 | THR | D | 122 | 64.463 | 56.273 | 89.190 | 1.00 | 52.62 | O |
| ATOM | 14054 | CG2 | THR | D | 122 | 65.040 | 55.501 | 91.265 | 1.00 | 55.87 | C |
| ATOM | 14058 | C | THR | D | 122 | 65.900 | 58.103 | 92.036 | 1.00 | 54.14 | C |
| ATOM | 14059 | O | THR | D | 122 | 67.082 | 58.370 | 92.070 | 1.00 | 57.74 | O |
| ATOM | 14060 | N | LYS | D | 123 | 65.155 | 57.970 | 93.101 | 1.00 | 52.70 | N |
| ATOM | 14062 | CA | LYS | D | 123 | 65.617 | 58.079 | 94.434 | 1.00 | 54.77 | C |
| ATOM | 14064 | CB | LYS | D | 123 | 65.602 | 59.545 | 94.866 | 1.00 | 55.21 | C |
| ATOM | 14067 | CG | LYS | D | 123 | 65.630 | 59.764 | 96.421 | 1.00 | 58.84 | C |
| ATOM | 14070 | CD | LYS | D | 123 | 66.294 | 61.098 | 96.875 | 1.00 | 62.89 | C |
| ATOM | 14073 | CE | LYS | D | 123 | 66.371 | 61.121 | 98.457 | 1.00 | 68.82 | C |
| ATOM | 14076 | NZ | LYS | D | 123 | 67.102 | 62.300 | 99.097 | 1.00 | 74.24 | N |
| ATOM | 14080 | C | LYS | D | 123 | 64.761 | 57.241 | 95.373 | 1.00 | 54.21 | C |
| ATOM | 14081 | O | LYS | D | 123 | 63.536 | 57.235 | 95.342 | 1.00 | 52.84 | O |
| ATOM | 14082 | N | GLY | D | 124 | 65.417 | 56.518 | 96.234 | 1.00 | 57.38 | N |
| ATOM | 14084 | CA | GLY | D | 124 | 64.733 | 55.809 | 97.291 | 1.00 | 58.45 | C |
| ATOM | 14087 | C | GLY | D | 124 | 64.316 | 56.758 | 98.400 | 1.00 | 58.43 | C |
| ATOM | 14088 | O | GLY | D | 124 | 64.894 | 57.864 | 98.560 | 1.00 | 58.61 | O |
| ATOM | 14089 | N | PRO | D | 125 | 63.310 | 56.315 | 99.155 | 1.00 | 58.47 | N |
| ATOM | 14090 | CA | PRO | D | 125 | 62.713 | 57.103 | 100.230 | 1.00 | 58.38 | C |
| ATOM | 14092 | CB | PRO | D | 125 | 61.436 | 56.350 | 100.539 | 1.00 | 57.92 | C |
| ATOM | 14095 | CG | PRO | D | 125 | 61.782 | 54.878 | 100.208 | 1.00 | 59.26 | C |
| ATOM | 14098 | CD | PRO | D | 125 | 62.690 | 54.979 | 99.035 | 1.00 | 59.00 | C |
| ATOM | 14101 | C | PRO | D | 125 | 63.552 | 57.038 | 101.469 | 1.00 | 62.83 | C |
| ATOM | 14102 | O | PRO | D | 125 | 64.321 | 56.073 | 101.644 | 1.00 | 64.70 | O |
| ATOM | 14103 | N | SER | D | 126 | 63.389 | 58.069 | 102.297 | 1.00 | 63.66 | N |
| ATOM | 14105 | CA | SER | D | 126 | 63.821 | 58.051 | 103.670 | 1.00 | 68.27 | C |
| ATOM | 14107 | CB | SER | D | 126 | 64.670 | 59.277 | 104.016 | 1.00 | 71.00 | C |
| ATOM | 14110 | OG | SER | D | 126 | 63.979 | 60.445 | 103.680 | 1.00 | 70.40 | O |
| ATOM | 14112 | C | SER | D | 126 | 62.520 | 57.927 | 104.480 | 1.00 | 67.10 | C |
| ATOM | 14113 | O | SER | D | 126 | 61.438 | 58.220 | 103.960 | 1.00 | 63.05 | O |
| ATOM | 14114 | N | VAL | D | 127 | 62.644 | 57.394 | 105.709 | 1.00 | 70.81 | N |
| ATOM | 14116 | CA | VAL | D | 127 | 61.531 | 56.782 | 106.473 | 1.00 | 70.08 | C |
| ATOM | 14118 | CB | VAL | D | 127 | 61.577 | 55.231 | 106.423 | 1.00 | 71.07 | C |
| ATOM | 14120 | CG1 | VAL | D | 127 | 60.469 | 54.669 | 107.223 | 1.00 | 72.19 | C |
| ATOM | 14124 | CG2 | VAL | D | 127 | 61.482 | 54.719 | 105.003 | 1.00 | 66.98 | C |
| ATOM | 14128 | C | VAL | D | 127 | 61.614 | 57.164 | 107.935 | 1.00 | 73.31 | C |
| ATOM | 14129 | O | VAL | D | 127 | 62.406 | 56.660 | 108.598 | 1.00 | 76.86 | O |
| ATOM | 14130 | N | PHE | D | 128 | 60.795 | 58.080 | 108.402 | 1.00 | 72.76 | N |
| ATOM | 14132 | CA | PHE | D | 128 | 60.755 | 58.481 | 109.789 | 1.00 | 77.52 | C |
| ATOM | 14134 | CB | PHE | D | 128 | 60.660 | 60.012 | 109.858 | 1.00 | 77.44 | C |
| ATOM | 14137 | CG | PHE | D | 128 | 61.762 | 60.709 | 109.068 | 1.00 | 78.35 | C |
| ATOM | 14138 | CD1 | PHE | D | 128 | 61.473 | 61.588 | 108.023 | 1.00 | 74.99 | C |
| ATOM | 14140 | CE1 | PHE | D | 128 | 62.471 | 62.155 | 107.297 | 1.00 | 73.17 | C |
| ATOM | 14142 | CZ | PHE | D | 128 | 63.765 | 61.868 | 107.582 | 1.00 | 77.82 | C |
| ATOM | 14144 | CE2 | PHE | D | 128 | 64.074 | 61.016 | 108.582 | 1.00 | 80.68 | C |
| ATOM | 14146 | CD2 | PHE | D | 128 | 63.089 | 60.430 | 109.318 | 1.00 | 80.90 | C |
| ATOM | 14148 | C | PHE | D | 128 | 59.576 | 57.790 | 110.461 | 1.00 | 78.03 | C |
| ATOM | 14149 | O | PHE | D | 128 | 58.667 | 57.365 | 109.780 | 1.00 | 74.02 | O |
| ATOM | 14150 | N | PRO | D | 129 | 59.604 | 57.611 | 111.781 | 1.00 | 84.09 | N |
| ATOM | 14151 | CA | PRO | D | 129 | 58.457 | 57.042 | 112.494 | 1.00 | 85.22 | C |
| ATOM | 14153 | CB | PRO | D | 129 | 59.078 | 56.527 | 113.776 | 1.00 | 91.56 | C |
| ATOM | 14156 | CG | PRO | D | 129 | 60.132 | 57.546 | 114.071 | 1.00 | 94.86 | C |
| ATOM | 14159 | CD | PRO | D | 129 | 60.703 | 57.940 | 112.717 | 1.00 | 90.22 | C |
| ATOM | 14162 | C | PRO | D | 129 | 57.522 | 58.179 | 112.818 | 1.00 | 85.06 | C |
| ATOM | 14163 | O | PRO | D | 129 | 57.900 | 59.302 | 112.572 | 1.00 | 85.76 | O |
| ATOM | 14164 | N | LEU | D | 130 | 56.343 | 57.890 | 113.342 | 1.00 | 85.59 | N |
| ATOM | 14166 | CA | LEU | D | 130 | 55.394 | 58.881 | 113.824 | 1.00 | 85.82 | C |
| ATOM | 14168 | CB | LEU | D | 130 | 54.239 | 59.068 | 112.847 | 1.00 | 80.81 | C |
| ATOM | 14171 | CG | LEU | D | 130 | 54.384 | 59.615 | 111.434 | 1.00 | 76.34 | C |
| ATOM | 14173 | CD1 | LEU | D | 130 | 53.028 | 59.517 | 110.732 | 1.00 | 73.31 | C |
| ATOM | 14177 | CD2 | LEU | D | 130 | 54.855 | 61.068 | 111.414 | 1.00 | 77.79 | C |
| ATOM | 14181 | C | LEU | D | 130 | 54.860 | 58.224 | 115.070 | 1.00 | 90.47 | C |
| ATOM | 14182 | O | LEU | D | 130 | 53.815 | 57.574 | 115.037 | 1.00 | 89.75 | O |
| ATOM | 14183 | N | ALA | D | 131 | 55.601 | 58.344 | 116.162 | 1.00 | 96.32 | N |
| ATOM | 14185 | CA | ALA | D | 131 | 55.347 | 57.554 | 117.353 | 1.00 | 101.04 | C |
| ATOM | 14187 | CB | ALA | D | 131 | 56.545 | 57.608 | 118.237 | 1.00 | 107.25 | C |
| ATOM | 14191 | C | ALA | D | 131 | 54.118 | 58.036 | 118.109 | 1.00 | 102.96 | C |
| ATOM | 14192 | O | ALA | D | 131 | 53.813 | 59.224 | 118.068 | 1.00 | 101.97 | O |
| ATOM | 14193 | N | PRO | D | 132 | 53.447 | 57.125 | 118.830 | 1.00 | 106.22 | N |
| ATOM | 14194 | CA | PRO | D | 132 | 52.215 | 57.439 | 119.582 | 1.00 | 108.61 | C |
| ATOM | 14196 | CB | PRO | D | 132 | 51.637 | 56.027 | 119.870 | 1.00 | 109.88 | C |
| ATOM | 14199 | CG | PRO | D | 132 | 52.854 | 55.128 | 120.013 | 1.00 | 111.73 | C |
| ATOM | 14202 | CD | PRO | D | 132 | 53.831 | 55.703 | 119.007 | 1.00 | 108.34 | C |
| ATOM | 14205 | C | PRO | D | 132 | 52.484 | 58.180 | 120.916 | 1.00 | 115.60 | C |
| ATOM | 14206 | O | PRO | D | 132 | 51.947 | 57.773 | 121.972 | 1.00 | 120.74 | O |
| ATOM | 14207 | N | SER | D | 133 | 53.261 | 59.267 | 120.859 | 1.00 | 116.62 | N |
| ATOM | 14209 | CA | SER | D | 133 | 54.103 | 59.666 | 122.000 | 1.00 | 124.00 | C |
| ATOM | 14211 | CB | SER | D | 133 | 55.207 | 60.712 | 121.636 | 1.00 | 124.54 | C |
| ATOM | 14214 | OG | SER | D | 133 | 54.848 | 61.677 | 120.645 | 1.00 | 118.70 | O |
| ATOM | 14216 | C | SER | D | 133 | 53.234 | 60.103 | 123.172 | 1.00 | 129.46 | C |
| ATOM | 14217 | O | SER | D | 133 | 52.147 | 60.677 | 122.931 | 1.00 | 127.35 | O |
| ATOM | 14218 | N | SER | D | 134 | 53.726 | 59.793 | 124.396 | 1.00 | 136.79 | N |
| ATOM | 14220 | CA | SER | D | 134 | 52.982 | 59.796 | 125.685 | 1.00 | 143.02 | C |
| ATOM | 14222 | CB | SER | D | 134 | 53.599 | 60.774 | 126.704 | 1.00 | 150.95 | C |
| ATOM | 14225 | OG | SER | D | 134 | 53.463 | 62.111 | 126.259 | 1.00 | 149.72 | O |
| ATOM | 14227 | C | SER | D | 134 | 51.470 | 60.022 | 125.546 | 1.00 | 140.00 | C |
| ATOM | 14228 | O | SER | D | 134 | 50.863 | 60.833 | 126.267 | 1.00 | 144.16 | O |
| ATOM | 14229 | N | LYS | D | 135 | 50.881 | 59.254 | 124.626 | 1.00 | 133.42 | N |
| ATOM | 14231 | CA | LYS | D | 135 | 49.472 | 59.353 | 124.290 | 1.00 | 130.16 | C |
| ATOM | 14233 | CB | LYS | D | 135 | 49.180 | 58.611 | 122.963 | 1.00 | 122.30 | C |
| ATOM | 14240 | C | LYS | D | 135 | 48.725 | 58.746 | 125.472 | 1.00 | 135.96 | C |
| ATOM | 14241 | O | LYS | D | 135 | 48.452 | 57.551 | 125.475 | 1.00 | 135.99 | O |
| ATOM | 14242 | N | SER | D | 136 | 48.443 | 59.541 | 126.506 | 1.00 | 141.90 | N |
| ATOM | 14244 | CA | SER | D | 136 | 47.762 | 58.997 | 127.683 | 1.00 | 148.08 | C |
| ATOM | 14246 | CB | SER | D | 136 | 48.003 | 59.872 | 128.938 | 1.00 | 156.17 | C |
| ATOM | 14249 | OG | SER | D | 136 | 47.204 | 59.449 | 130.043 | 1.00 | 160.68 | O |
| ATOM | 14251 | C | SER | D | 136 | 46.252 | 58.781 | 127.358 | 1.00 | 145.76 | C |
| ATOM | 14252 | O | SER | D | 136 | 45.478 | 59.749 | 127.277 | 1.00 | 145.97 | O |
| ATOM | 14253 | N | THR | D | 137 | 45.887 | 57.508 | 127.105 | 1.00 | 143.97 | N |
| ATOM | 14255 | CA | THR | D | 137 | 44.495 | 57.012 | 126.990 | 1.00 | 142.86 | C |
| ATOM | 14257 | CB | THR | D | 137 | 43.968 | 56.999 | 125.484 | 1.00 | 134.89 | C |
| ATOM | 14259 | OG1 | THR | D | 137 | 44.846 | 57.734 | 124.605 | 1.00 | 129.83 | O |
| ATOM | 14261 | CG2 | THR | D | 137 | 42.567 | 57.671 | 125.358 | 1.00 | 134.26 | C |
| ATOM | 14265 | C | THR | D | 137 | 44.440 | 55.570 | 127.549 | 1.00 | 146.23 | C |
| ATOM | 14266 | O | THR | D | 137 | 44.505 | 54.608 | 126.769 | 1.00 | 142.87 | O |
| ATOM | 14267 | N | SER | D | 138 | 44.364 | 55.417 | 128.874 | 1.00 | 153.23 | N |
| ATOM | 14269 | CA | SER | D | 138 | 44.243 | 54.088 | 129.508 | 1.00 | 157.67 | C |
| ATOM | 14271 | CB | SER | D | 138 | 43.834 | 54.215 | 131.017 | 1.00 | 166.65 | C |
| ATOM | 14274 | OG | SER | D | 138 | 42.567 | 53.646 | 131.354 | 1.00 | 167.36 | O |
| ATOM | 14276 | C | SER | D | 138 | 43.278 | 53.162 | 128.739 | 1.00 | 154.58 | C |
| ATOM | 14277 | O | SER | D | 138 | 43.711 | 52.177 | 128.126 | 1.00 | 151.95 | O |
| ATOM | 14278 | N | GLY | D | 139 | 41.984 | 53.513 | 128.758 | 1.00 | 155.39 | N |
| ATOM | 14280 | CA | GLY | D | 139 | 40.916 | 52.737 | 128.132 | 1.00 | 153.62 | C |
| ATOM | 14283 | C | GLY | D | 139 | 40.310 | 53.374 | 126.872 | 1.00 | 147.16 | C |
| ATOM | 14284 | O | GLY | D | 139 | 39.078 | 53.540 | 126.851 | 1.00 | 148.77 | O |
| ATOM | 14285 | N | GLY | D | 140 | 41.159 | 53.757 | 125.887 | 1.00 | 140.58 | N |
| ATOM | 14287 | CA | GLY | D | 140 | 40.785 | 54.070 | 124.501 | 1.00 | 133.65 | C |
| ATOM | 14290 | C | GLY | D | 140 | 41.900 | 53.685 | 123.488 | 1.00 | 128.29 | C |
| ATOM | 14291 | O | GLY | D | 140 | 43.034 | 53.341 | 123.877 | 1.00 | 129.62 | O |
| ATOM | 14292 | N | THR | D | 141 | 41.622 | 53.770 | 122.181 | 1.00 | 122.35 | N |
| ATOM | 14294 | CA | THR | D | 141 | 42.543 | 53.206 | 121.174 | 1.00 | 117.27 | C |
| ATOM | 14296 | CB | THR | D | 141 | 41.749 | 52.518 | 120.026 | 1.00 | 114.37 | C |
| ATOM | 14298 | OG1 | THR | D | 141 | 41.283 | 51.237 | 120.468 | 1.00 | 118.95 | O |
| ATOM | 14300 | CG2 | THR | D | 141 | 42.640 | 52.107 | 118.885 | 1.00 | 110.43 | C |
| ATOM | 14304 | C | THR | D | 141 | 43.460 | 54.289 | 120.658 | 1.00 | 112.42 | C |
| ATOM | 14305 | O | THR | D | 141 | 43.007 | 55.385 | 120.437 | 1.00 | 110.84 | O |
| ATOM | 14306 | N | ALA | D | 142 | 44.744 | 53.980 | 120.495 | 1.00 | 110.72 | N |
| ATOM | 14308 | CA | ALA | D | 142 | 45.741 | 54.969 | 120.088 | 1.00 | 107.63 | C |
| ATOM | 14310 | CB | ALA | D | 142 | 46.854 | 55.047 | 121.088 | 1.00 | 111.82 | C |
| ATOM | 14314 | C | ALA | D | 142 | 46.308 | 54.630 | 118.726 | 1.00 | 102.27 | C |
| ATOM | 14315 | O | ALA | D | 142 | 46.122 | 53.503 | 118.238 | 1.00 | 101.65 | O |
| ATOM | 14316 | N | ALA | D | 143 | 47.020 | 55.610 | 118.149 | 1.00 | 98.80 | N |
| ATOM | 14318 | CA | ALA | D | 143 | 47.476 | 55.602 | 116.748 | 1.00 | 93.39 | C |
| ATOM | 14320 | CB | ALA | D | 143 | 46.600 | 56.585 | 115.878 | 1.00 | 89.96 | C |
| ATOM | 14324 | C | ALA | D | 143 | 48.970 | 55.943 | 116.551 | 1.00 | 92.18 | C |
| ATOM | 14325 | O | ALA | D | 143 | 49.568 | 56.769 | 117.278 | 1.00 | 93.80 | O |
| ATOM | 14326 | N | LEU | D | 144 | 49.547 | 55.316 | 115.529 | 1.00 | 88.87 | N |
| ATOM | 14328 | CA | LEU | D | 144 | 50.950 | 55.507 | 115.213 | 1.00 | 88.48 | C |
| ATOM | 14330 | CB | LEU | D | 144 | 51.793 | 54.604 | 116.101 | 1.00 | 93.10 | C |
| ATOM | 14333 | CG | LEU | D | 144 | 51.319 | 53.166 | 116.222 | 1.00 | 94.60 | C |
| ATOM | 14335 | CD1 | LEU | D | 144 | 51.776 | 52.328 | 115.026 | 1.00 | 91.71 | C |
| ATOM | 14339 | CD2 | LEU | D | 144 | 51.849 | 52.610 | 117.557 | 1.00 | 101.82 | C |
| ATOM | 14343 | C | LEU | D | 144 | 51.243 | 55.237 | 113.737 | 1.00 | 84.16 | C |
| ATOM | 14344 | O | LEU | D | 144 | 50.331 | 54.826 | 112.998 | 1.00 | 81.75 | O |
| ATOM | 14345 | N | GLY | D | 145 | 52.504 | 55.438 | 113.316 | 1.00 | 83.52 | N |
| ATOM | 14347 | CA | GLY | D | 145 | 52.856 | 55.270 | 111.913 | 1.00 | 79.91 | C |
| ATOM | 14350 | C | GLY | D | 145 | 54.282 | 55.538 | 111.367 | 1.00 | 79.57 | C |
| ATOM | 14351 | O | GLY | D | 145 | 55.304 | 55.437 | 112.066 | 1.00 | 83.32 | O |
| ATOM | 14352 | N | CYS | D | 146 | 54.312 | 55.893 | 110.075 | 1.00 | 75.22 | N |
| ATOM | 14354 | CA | CYS | D | 146 | 55.508 | 55.855 | 109.206 | 1.00 | 73.84 | C |
| ATOM | 14356 | CB | CYS | D | 146 | 55.664 | 54.503 | 108.451 | 1.00 | 73.43 | C |
| ATOM | 14359 | SG | CYS | D | 146 | 56.308 | 53.216 | 109.520 | 1.00 | 81.52 | S |
| ATOM | 14360 | C | CYS | D | 146 | 55.384 | 56.912 | 108.144 | 1.00 | 69.05 | C |
| ATOM | 14361 | O | CYS | D | 146 | 54.402 | 56.950 | 107.391 | 1.00 | 65.38 | O |
| ATOM | 14362 | N | LEU | D | 147 | 56.406 | 57.747 | 108.065 | 1.00 | 68.91 | N |
| ATOM | 14364 | CA | LEU | D | 147 | 56.432 | 58.804 | 107.109 | 1.00 | 65.13 | C |
| ATOM | 14366 | CB | LEU | D | 147 | 56.963 | 60.025 | 107.764 | 1.00 | 67.65 | C |
| ATOM | 14369 | CG | LEU | D | 147 | 57.069 | 61.227 | 106.873 | 1.00 | 67.06 | C |
| ATOM | 14371 | CD1 | LEU | D | 147 | 55.724 | 61.483 | 106.297 | 1.00 | 65.61 | C |
| ATOM | 14375 | CD2 | LEU | D | 147 | 57.559 | 62.440 | 107.688 | 1.00 | 71.20 | C |
| ATOM | 14379 | C | LEU | D | 147 | 57.370 | 58.331 | 106.083 | 1.00 | 63.16 | C |
| ATOM | 14380 | O | LEU | D | 147 | 58.552 | 58.304 | 106.328 | 1.00 | 64.33 | O |
| ATOM | 14381 | N | VAL | D | 148 | 56.830 | 57.899 | 104.943 | 1.00 | 60.56 | N |
| ATOM | 14383 | CA | VAL | D | 148 | 57.671 | 57.588 | 103.762 | 1.00 | 59.44 | C |
| ATOM | 14385 | CB | VAL | D | 148 | 57.170 | 56.371 | 103.022 | 1.00 | 57.55 | C |
| ATOM | 14387 | CG1 | VAL | D | 148 | 58.149 | 56.036 | 101.950 | 1.00 | 56.88 | C |
| ATOM | 14391 | CG2 | VAL | D | 148 | 57.071 | 55.207 | 103.985 | 1.00 | 60.14 | C |
| ATOM | 14395 | C | VAL | D | 148 | 57.880 | 58.789 | 102.783 | 1.00 | 57.27 | C |
| ATOM | 14396 | O | VAL | D | 148 | 56.988 | 59.143 | 102.012 | 1.00 | 56.19 | O |
| ATOM | 14397 | N | LYS | D | 149 | 59.063 | 59.372 | 102.818 | 1.00 | 57.66 | N |
| ATOM | 14399 | CA | LYS | D | 149 | 59.293 | 60.687 | 102.272 | 1.00 | 56.80 | C |
| ATOM | 14401 | CB | LYS | D | 149 | 59.604 | 61.579 | 103.479 | 1.00 | 60.19 | C |
| ATOM | 14404 | CG | LYS | D | 149 | 60.038 | 63.016 | 103.218 | 1.00 | 62.14 | C |
| ATOM | 14407 | CD | LYS | D | 149 | 59.519 | 63.986 | 104.319 | 1.00 | 64.95 | C |
| ATOM | 14410 | CE | LYS | D | 149 | 59.517 | 65.451 | 103.856 | 1.00 | 66.61 | C |
| ATOM | 14413 | NZ | LYS | D | 149 | 60.933 | 65.996 | 103.508 | 1.00 | 69.35 | N |
| ATOM | 14417 | C | LYS | D | 149 | 60.435 | 60.715 | 101.246 | 1.00 | 56.13 | C |
| ATOM | 14418 | O | LYS | D | 149 | 61.365 | 59.926 | 101.268 | 1.00 | 58.22 | O |
| ATOM | 14419 | N | ASP | D | 150 | 60.363 | 61.652 | 100.340 | 1.00 | 54.55 | N |
| ATOM | 14421 | CA | ASP | D | 150 | 61.444 | 61.929 | 99.384 | 1.00 | 54.34 | C |
| ATOM | 14423 | CB | ASP | D | 150 | 62.629 | 62.585 | 100.090 | 1.00 | 58.17 | C |
| ATOM | 14426 | CG | ASP | D | 150 | 62.264 | 63.912 | 100.695 | 1.00 | 60.09 | C |
| ATOM | 14427 | OD1 | ASP | D | 150 | 62.982 | 64.376 | 101.590 | 1.00 | 69.14 | O |
| ATOM | 14428 | OD2 | ASP | D | 150 | 61.267 | 64.570 | 100.383 | 1.00 | 58.91 | O |
| ATOM | 14429 | C | ASP | D | 150 | 61.896 | 60.772 | 98.527 | 1.00 | 52.77 | C |
| ATOM | 14430 | O | ASP | D | 150 | 63.026 | 60.348 | 98.599 | 1.00 | 54.66 | O |
| ATOM | 14431 | N | TYR | D | 151 | 60.981 | 60.295 | 97.682 | 1.00 | 50.75 | N |
| ATOM | 14433 | CA | TYR | D | 151 | 61.252 | 59.228 | 96.706 | 1.00 | 49.16 | C |
| ATOM | 14435 | CB | TYR | D | 151 | 60.723 | 57.880 | 97.179 | 1.00 | 49.80 | C |
| ATOM | 14438 | CG | TYR | D | 151 | 59.222 | 57.790 | 97.252 | 1.00 | 48.09 | C |
| ATOM | 14439 | CD1 | TYR | D | 151 | 58.483 | 57.376 | 96.154 | 1.00 | 46.12 | C |
| ATOM | 14441 | CE1 | TYR | D | 151 | 57.135 | 57.321 | 96.171 | 1.00 | 45.49 | C |
| ATOM | 14443 | CZ | TYR | D | 151 | 56.438 | 57.658 | 97.307 | 1.00 | 45.19 | C |
| ATOM | 14444 | OH | TYR | D | 151 | 55.092 | 57.521 | 97.328 | 1.00 | 43.36 | O |
| ATOM | 14446 | CE2 | TYR | D | 151 | 57.101 | 58.077 | 98.410 | 1.00 | 49.83 | C |
| ATOM | 14448 | CD2 | TYR | D | 151 | 58.545 | 58.130 | 98.391 | 1.00 | 49.84 | C |
| ATOM | 14450 | C | TYR | D | 151 | 60.657 | 59.567 | 95.382 | 1.00 | 46.65 | C |
| ATOM | 14451 | O | TYR | D | 151 | 59.792 | 60.426 | 95.271 | 1.00 | 44.77 | O |
| ATOM | 14452 | N | PHE | D | 152 | 61.155 | 58.867 | 94.379 | 1.00 | 46.98 | N |
| ATOM | 14454 | CA | PHE | D | 152 | 60.724 | 59.046 | 93.020 | 1.00 | 46.06 | C |
| ATOM | 14456 | CB | PHE | D | 152 | 61.221 | 60.378 | 92.532 | 1.00 | 46.70 | C |
| ATOM | 14459 | CG | PHE | D | 152 | 60.649 | 60.766 | 91.240 | 1.00 | 46.30 | C |
| ATOM | 14460 | CD1 | PHE | D | 152 | 61.258 | 60.423 | 90.090 | 1.00 | 47.89 | C |
| ATOM | 14462 | CE1 | PHE | D | 152 | 60.726 | 60.772 | 88.909 | 1.00 | 47.13 | C |
| ATOM | 14464 | CZ | PHE | D | 152 | 59.636 | 61.473 | 88.857 | 1.00 | 43.58 | C |
| ATOM | 14466 | CE2 | PHE | D | 152 | 59.024 | 61.811 | 89.990 | 1.00 | 45.67 | C |
| ATOM | 14468 | CD2 | PHE | D | 152 | 59.510 | 61.472 | 91.174 | 1.00 | 44.91 | C |
| ATOM | 14470 | C | PHE | D | 152 | 61.265 | 57.933 | 92.107 | 1.00 | 47.13 | C |
| ATOM | 14471 | O | PHE | D | 152 | 62.368 | 57.460 | 92.281 | 1.00 | 46.91 | O |
| ATOM | 14472 | N | PRO | D | 153 | 60.458 | 57.446 | 91.172 | 1.00 | 47.74 | N |
| ATOM | 14473 | CA | PRO | D | 153 | 59.064 | 57.821 | 91.009 | 1.00 | 46.84 | C |
| ATOM | 14475 | CB | PRO | D | 153 | 58.855 | 57.629 | 89.519 | 1.00 | 47.99 | C |
| ATOM | 14478 | CG | PRO | D | 153 | 59.782 | 56.422 | 89.160 | 1.00 | 49.21 | C |
| ATOM | 14481 | CD | PRO | D | 153 | 60.823 | 56.380 | 90.221 | 1.00 | 49.16 | C |
| ATOM | 14484 | C | PRO | D | 153 | 58.259 | 56.813 | 91.759 | 1.00 | 46.89 | C |
| ATOM | 14485 | O | PRO | D | 153 | 58.853 | 56.011 | 92.434 | 1.00 | 46.53 | O |
| ATOM | 14486 | N | GLU | D | 154 | 56.945 | 56.877 | 91.635 | 1.00 | 47.75 | N |
| ATOM | 14488 | CA | GLU | D | 154 | 56.045 | 55.904 | 92.262 | 1.00 | 49.53 | C |
| ATOM | 14490 | CB | GLU | D | 154 | 54.615 | 56.391 | 92.071 | 1.00 | 49.61 | C |
| ATOM | 14493 | CG | GLU | D | 154 | 54.229 | 57.558 | 92.949 | 1.00 | 48.55 | C |
| ATOM | 14496 | CD | GLU | D | 154 | 53.234 | 57.082 | 94.005 | 1.00 | 51.59 | C |
| ATOM | 14497 | OE1 | GLU | D | 154 | 53.672 | 56.795 | 95.160 | 1.00 | 50.24 | O |
| ATOM | 14498 | OE2 | GLU | D | 154 | 52.016 | 56.961 | 93.650 | 1.00 | 52.02 | O |
| ATOM | 14499 | C | GLU | D | 154 | 56.182 | 54.533 | 91.593 | 1.00 | 51.99 | C |
| ATOM | 14500 | O | GLU | D | 154 | 56.757 | 54.438 | 90.491 | 1.00 | 52.94 | O |
| ATOM | 14501 | N | PRO | D | 155 | 55.706 | 53.469 | 92.229 | 1.00 | 54.47 | N |
| ATOM | 14502 | CA | PRO | D | 155 | 55.067 | 53.470 | 93.560 | 1.00 | 55.09 | C |
| ATOM | 14504 | CB | PRO | D | 155 | 53.979 | 52.480 | 93.367 | 1.00 | 56.62 | C |
| ATOM | 14507 | CG | PRO | D | 155 | 54.662 | 51.449 | 92.460 | 1.00 | 58.94 | C |
| ATOM | 14510 | CD | PRO | D | 155 | 55.738 | 52.112 | 91.677 | 1.00 | 57.37 | C |
| ATOM | 14513 | C | PRO | D | 155 | 55.902 | 52.909 | 94.714 | 1.00 | 56.44 | C |
| ATOM | 14514 | O | PRO | D | 155 | 56.987 | 52.293 | 94.496 | 1.00 | 57.94 | O |
| ATOM | 14515 | N | VAL | D | 156 | 55.356 | 53.104 | 95.921 | 1.00 | 55.95 | N |
| ATOM | 14517 | CA | VAL | D | 156 | 55.900 | 52.510 | 97.110 | 1.00 | 58.09 | C |
| ATOM | 14519 | CB | VAL | D | 156 | 56.074 | 53.534 | 98.199 | 1.00 | 57.30 | C |
| ATOM | 14521 | CG1 | VAL | D | 156 | 56.640 | 52.887 | 99.512 | 1.00 | 60.92 | C |
| ATOM | 14525 | CG2 | VAL | D | 156 | 57.072 | 54.513 | 97.769 | 1.00 | 57.85 | C |
| ATOM | 14529 | C | VAL | D | 156 | 54.907 | 51.505 | 97.595 | 1.00 | 60.29 | C |
| ATOM | 14530 | O | VAL | D | 156 | 53.748 | 51.612 | 97.316 | 1.00 | 61.45 | O |
| ATOM | 14531 | N | THR | D | 157 | 55.373 | 50.518 | 98.313 | 1.00 | 62.28 | N |
| ATOM | 14533 | CA | THR | D | 157 | 54.531 | 49.596 | 98.993 | 1.00 | 64.84 | C |
| ATOM | 14535 | CB | THR | D | 157 | 55.070 | 48.221 | 98.666 | 1.00 | 68.70 | C |
| ATOM | 14537 | OG1 | THR | D | 157 | 54.767 | 47.907 | 97.300 | 1.00 | 68.61 | O |
| ATOM | 14539 | CG2 | THR | D | 157 | 54.377 | 47.166 | 99.467 | 1.00 | 74.43 | C |
| ATOM | 14543 | C | THR | D | 157 | 54.791 | 49.880 | 100.432 | 1.00 | 65.43 | C |
| ATOM | 14544 | O | THR | D | 157 | 55.929 | 50.121 | 100.772 | 1.00 | 67.18 | O |
| ATOM | 14545 | N | VAL | D | 158 | 53.792 | 49.826 | 101.297 | 1.00 | 66.13 | N |
| ATOM | 14547 | CA | VAL | D | 158 | 54.027 | 49.793 | 102.753 | 1.00 | 67.84 | C |
| ATOM | 14549 | CB | VAL | D | 158 | 53.496 | 51.066 | 103.435 | 1.00 | 65.69 | C |
| ATOM | 14551 | CG1 | VAL | D | 158 | 53.812 | 51.053 | 104.905 | 1.00 | 67.82 | C |
| ATOM | 14555 | CG2 | VAL | D | 158 | 54.053 | 52.331 | 102.774 | 1.00 | 61.61 | C |
| ATOM | 14559 | C | VAL | D | 158 | 53.277 | 48.599 | 103.351 | 1.00 | 72.19 | C |
| ATOM | 14560 | O | VAL | D | 158 | 52.314 | 48.156 | 102.776 | 1.00 | 73.95 | O |
| ATOM | 14561 | N | SER | D | 159 | 53.681 | 48.090 | 104.507 | 1.00 | 75.68 | N |
| ATOM | 14563 | CA | SER | D | 159 | 52.984 | 46.954 | 105.155 | 1.00 | 80.05 | C |
| ATOM | 14565 | CB | SER | D | 159 | 53.231 | 45.659 | 104.387 | 1.00 | 83.26 | C |
| ATOM | 14568 | OG | SER | D | 159 | 54.603 | 45.300 | 104.525 | 1.00 | 84.89 | O |
| ATOM | 14570 | C | SER | D | 159 | 53.544 | 46.773 | 106.546 | 1.00 | 83.51 | C |
| ATOM | 14571 | O | SER | D | 159 | 54.718 | 47.089 | 106.775 | 1.00 | 83.52 | O |
| ATOM | 14572 | N | TRP | D | 160 | 52.768 | 46.211 | 107.471 | 1.00 | 87.04 | N |
| ATOM | 14574 | CA | TRP | D | 160 | 53.284 | 46.101 | 108.846 | 1.00 | 90.70 | C |
| ATOM | 14576 | CB | TRP | D | 160 | 52.378 | 46.873 | 109.763 | 1.00 | 89.83 | C |
| ATOM | 14579 | CG | TRP | D | 160 | 52.246 | 48.303 | 109.409 | 1.00 | 84.69 | C |
| ATOM | 14580 | CD1 | TRP | D | 160 | 51.543 | 48.842 | 108.352 | 1.00 | 79.29 | C |
| ATOM | 14582 | NE1 | TRP | D | 160 | 51.623 | 50.213 | 108.391 | 1.00 | 73.78 | N |
| ATOM | 14584 | CE2 | TRP | D | 160 | 52.373 | 50.592 | 109.470 | 1.00 | 76.63 | C |
| ATOM | 14585 | CD2 | TRP | D | 160 | 52.788 | 49.408 | 110.131 | 1.00 | 83.36 | C |
| ATOM | 14586 | CE3 | TRP | D | 160 | 53.560 | 49.524 | 111.289 | 1.00 | 87.07 | C |
| ATOM | 14588 | CZ3 | TRP | D | 160 | 53.910 | 50.795 | 111.732 | 1.00 | 86.68 | C |
| ATOM | 14590 | CH2 | TRP | D | 160 | 53.494 | 51.947 | 111.041 | 1.00 | 80.65 | C |
| ATOM | 14592 | CZ2 | TRP | D | 160 | 52.723 | 51.864 | 109.916 | 1.00 | 75.76 | C |
| ATOM | 14594 | C | TRP | D | 160 | 53.529 | 44.669 | 109.400 | 1.00 | 97.32 | C |
| ATOM | 14595 | O | TRP | D | 160 | 52.681 | 43.805 | 109.261 | 1.00 | 100.42 | O |
| ATOM | 14596 | N | ASN | D | 161 | 54.708 | 44.447 | 110.002 | 1.00 | 100.50 | N |
| ATOM | 14598 | CA | ASN | D | 161 | 55.107 | 43.189 | 110.664 | 1.00 | 107.59 | C |
| ATOM | 14600 | CB | ASN | D | 161 | 54.168 | 42.853 | 111.837 | 1.00 | 111.38 | C |
| ATOM | 14603 | CG | ASN | D | 161 | 54.071 | 43.990 | 112.867 | 1.00 | 109.86 | C |
| ATOM | 14604 | OD1 | ASN | D | 161 | 53.451 | 43.837 | 113.914 | 1.00 | 113.63 | O |
| ATOM | 14605 | ND2 | ASN | D | 161 | 54.700 | 45.122 | 112.572 | 1.00 | 104.12 | N |
| ATOM | 14608 | C | ASN | D | 161 | 55.201 | 42.031 | 109.686 | 1.00 | 109.80 | C |
| ATOM | 14609 | O | ASN | D | 161 | 54.581 | 40.995 | 109.890 | 1.00 | 113.89 | O |
| ATOM | 14610 | N | SER | D | 162 | 56.001 | 42.232 | 108.633 | 1.00 | 107.26 | N |
| ATOM | 14612 | CA | SER | D | 162 | 55.962 | 41.418 | 107.389 | 1.00 | 108.05 | C |
| ATOM | 14614 | CB | SER | D | 162 | 56.905 | 40.169 | 107.464 | 1.00 | 115.28 | C |
| ATOM | 14617 | OG | SER | D | 162 | 56.533 | 39.196 | 108.446 | 1.00 | 122.27 | O |
| ATOM | 14619 | C | SER | D | 162 | 54.515 | 41.104 | 106.843 | 1.00 | 106.92 | C |
| ATOM | 14620 | O | SER | D | 162 | 54.284 | 40.170 | 106.071 | 1.00 | 109.12 | O |
| ATOM | 14621 | N | GLY | D | 163 | 53.558 | 41.941 | 107.206 | 1.00 | 103.43 | N |
| ATOM | 14623 | CA | GLY | D | 163 | 52.218 | 41.849 | 106.659 | 1.00 | 102.77 | C |
| ATOM | 14626 | C | GLY | D | 163 | 51.214 | 41.161 | 107.561 | 1.00 | 107.96 | C |
| ATOM | 14627 | O | GLY | D | 163 | 50.140 | 40.801 | 107.100 | 1.00 | 109.21 | O |
| ATOM | 14628 | N | ALA | D | 164 | 51.541 | 40.998 | 108.842 | 1.00 | 111.31 | N |
| ATOM | 14630 | CA | ALA | D | 164 | 50.641 | 40.355 | 109.775 | 1.00 | 116.24 | C |
| ATOM | 14632 | CB | ALA | D | 164 | 51.422 | 39.829 | 110.979 | 1.00 | 121.90 | C |
| ATOM | 14636 | C | ALA | D | 164 | 49.498 | 41.278 | 110.236 | 1.00 | 113.12 | C |
| ATOM | 14637 | O | ALA | D | 164 | 48.523 | 40.774 | 110.836 | 1.00 | 117.66 | O |
| ATOM | 14638 | N | LEU | D | 165 | 49.610 | 42.594 | 109.985 | 1.00 | 106.18 | N |
| ATOM | 14640 | CA | LEU | D | 165 | 48.572 | 43.596 | 110.366 | 1.00 | 103.35 | C |
| ATOM | 14642 | CB | LEU | D | 165 | 49.213 | 44.841 | 111.017 | 1.00 | 100.01 | C |
| ATOM | 14645 | CG | LEU | D | 165 | 49.884 | 44.675 | 112.393 | 1.00 | 104.07 | C |
| ATOM | 14647 | CD1 | LEU | D | 165 | 49.895 | 46.010 | 113.140 | 1.00 | 100.25 | C |
| ATOM | 14651 | CD2 | LEU | D | 165 | 49.215 | 43.516 | 113.304 | 1.00 | 113.44 | C |
| ATOM | 14655 | C | LEU | D | 165 | 47.666 | 44.060 | 109.206 | 1.00 | 99.38 | C |
| ATOM | 14656 | O | LEU | D | 165 | 48.126 | 44.281 | 108.107 | 1.00 | 95.88 | O |
| ATOM | 14657 | N | THR | D | 166 | 46.382 | 44.244 | 109.481 | 1.00 | 100.31 | N |
| ATOM | 14659 | CA | THR | D | 166 | 45.386 | 44.436 | 108.428 | 1.00 | 98.64 | C |
| ATOM | 14661 | CB | THR | D | 166 | 44.681 | 43.068 | 108.100 | 1.00 | 104.71 | C |
| ATOM | 14663 | OG1 | THR | D | 166 | 45.324 | 42.495 | 106.961 | 1.00 | 104.16 | O |
| ATOM | 14665 | CG2 | THR | D | 166 | 43.167 | 43.212 | 107.657 | 1.00 | 105.79 | C |
| ATOM | 14669 | C | THR | D | 166 | 44.388 | 45.506 | 108.810 | 1.00 | 96.21 | C |
| ATOM | 14670 | O | THR | D | 166 | 44.421 | 46.562 | 108.235 | 1.00 | 91.40 | O |
| ATOM | 14671 | N | SER | D | 167 | 43.492 | 45.242 | 109.755 | 1.00 | 100.00 | N |
| ATOM | 14673 | CA | SER | D | 167 | 42.552 | 46.277 | 110.121 | 1.00 | 98.66 | C |
| ATOM | 14675 | CB | SER | D | 167 | 41.333 | 45.736 | 110.879 | 1.00 | 103.89 | C |
| ATOM | 14678 | OG | SER | D | 167 | 41.735 | 45.120 | 112.053 | 1.00 | 106.75 | O |
| ATOM | 14680 | C | SER | D | 167 | 43.307 | 47.389 | 110.895 | 1.00 | 95.73 | C |
| ATOM | 14681 | O | SER | D | 167 | 44.117 | 47.131 | 111.821 | 1.00 | 96.71 | O |
| ATOM | 14682 | N | GLY | D | 168 | 43.027 | 48.623 | 110.464 | 1.00 | 91.77 | N |
| ATOM | 14684 | CA | GLY | D | 168 | 43.655 | 49.807 | 110.982 | 1.00 | 89.03 | C |
| ATOM | 14687 | C | GLY | D | 168 | 44.604 | 50.335 | 109.936 | 1.00 | 84.96 | C |
| ATOM | 14688 | O | GLY | D | 168 | 45.059 | 51.438 | 110.086 | 1.00 | 83.18 | O |
| ATOM | 14689 | N | VAL | D | 169 | 44.892 | 49.580 | 108.871 | 1.00 | 84.50 | N |
| ATOM | 14691 | CA | VAL | D | 169 | 45.986 | 49.932 | 107.935 | 1.00 | 80.36 | C |
| ATOM | 14693 | CB | VAL | D | 169 | 46.696 | 48.697 | 107.290 | 1.00 | 82.08 | C |
| ATOM | 14695 | CG1 | VAL | D | 169 | 47.829 | 49.109 | 106.355 | 1.00 | 76.99 | C |
| ATOM | 14699 | CG2 | VAL | D | 169 | 47.248 | 47.793 | 108.385 | 1.00 | 87.51 | C |
| ATOM | 14703 | C | VAL | D | 169 | 45.440 | 50.853 | 106.859 | 1.00 | 76.27 | C |
| ATOM | 14704 | O | VAL | D | 169 | 44.763 | 50.448 | 105.886 | 1.00 | 75.78 | O |
| ATOM | 14705 | N | HIS | D | 170 | 45.770 | 52.104 | 107.099 | 1.00 | 72.66 | N |
| ATOM | 14707 | CA | HIS | D | 170 | 45.447 | 53.183 | 106.249 | 1.00 | 69.38 | C |
| ATOM | 14709 | CB | HIS | D | 170 | 44.786 | 54.234 | 107.116 | 1.00 | 70.06 | C |
| ATOM | 14712 | CG | HIS | D | 170 | 43.316 | 54.039 | 107.318 | 1.00 | 71.85 | C |
| ATOM | 14713 | ND1 | HIS | D | 170 | 42.431 | 53.891 | 106.277 | 1.00 | 70.83 | N |
| ATOM | 14715 | CE1 | HIS | D | 170 | 41.200 | 53.816 | 106.759 | 1.00 | 75.91 | C |
| ATOM | 14717 | NE2 | HIS | D | 170 | 41.258 | 53.904 | 108.075 | 1.00 | 76.31 | N |
| ATOM | 14719 | CD2 | HIS | D | 170 | 42.571 | 54.040 | 108.449 | 1.00 | 74.79 | C |
| ATOM | 14721 | C | HIS | D | 170 | 46.730 | 53.740 | 105.599 | 1.00 | 65.87 | C |
| ATOM | 14722 | O | HIS | D | 170 | 47.590 | 54.326 | 106.240 | 1.00 | 64.33 | O |
| ATOM | 14723 | N | THR | D | 171 | 46.813 | 53.616 | 104.287 | 1.00 | 64.92 | N |
| ATOM | 14725 | CA | THR | D | 171 | 48.045 | 53.890 | 103.569 | 1.00 | 61.85 | C |
| ATOM | 14727 | CB | THR | D | 171 | 48.525 | 52.618 | 102.809 | 1.00 | 63.26 | C |
| ATOM | 14729 | OG1 | THR | D | 171 | 48.681 | 51.525 | 103.768 | 1.00 | 68.52 | O |
| ATOM | 14731 | CG2 | THR | D | 171 | 49.922 | 52.775 | 102.220 | 1.00 | 60.74 | C |
| ATOM | 14735 | C | THR | D | 171 | 48.066 | 55.083 | 102.701 | 1.00 | 57.66 | C |
| ATOM | 14736 | O | THR | D | 171 | 49.097 | 55.421 | 102.275 | 1.00 | 59.99 | O |
| ATOM | 14737 | N | PHE | D | 172 | 47.052 | 55.841 | 102.493 | 1.00 | 56.24 | N |
| ATOM | 14739 | CA | PHE | D | 172 | 47.309 | 57.278 | 102.015 | 1.00 | 53.44 | C |
| ATOM | 14741 | CB | PHE | D | 172 | 47.980 | 58.182 | 103.068 | 1.00 | 52.85 | C |
| ATOM | 14744 | CG | PHE | D | 172 | 47.131 | 58.461 | 104.272 | 1.00 | 56.06 | C |
| ATOM | 14745 | CD1 | PHE | D | 172 | 47.421 | 57.886 | 105.494 | 1.00 | 57.70 | C |
| ATOM | 14747 | CE1 | PHE | D | 172 | 46.598 | 58.125 | 106.593 | 1.00 | 61.30 | C |
| ATOM | 14749 | CZ | PHE | D | 172 | 45.466 | 58.949 | 106.488 | 1.00 | 61.98 | C |
| ATOM | 14751 | CE2 | PHE | D | 172 | 45.158 | 59.512 | 105.286 | 1.00 | 61.46 | C |
| ATOM | 14753 | CD2 | PHE | D | 172 | 46.002 | 59.285 | 104.175 | 1.00 | 58.64 | C |
| ATOM | 14755 | C | PHE | D | 172 | 48.040 | 57.484 | 100.684 | 1.00 | 50.18 | C |
| ATOM | 14756 | O | PHE | D | 172 | 49.188 | 57.152 | 100.499 | 1.00 | 48.95 | O |
| ATOM | 14757 | N | PRO | D | 173 | 47.323 | 57.960 | 99.703 | 1.00 | 51.03 | N |
| ATOM | 14758 | CA | PRO | D | 173 | 47.966 | 58.259 | 98.437 | 1.00 | 50.61 | C |
| ATOM | 14760 | CB | PRO | D | 173 | 46.828 | 58.841 | 97.589 | 1.00 | 51.50 | C |
| ATOM | 14763 | CG | PRO | D | 173 | 45.802 | 59.112 | 98.483 | 1.00 | 53.99 | C |
| ATOM | 14766 | CD | PRO | D | 173 | 45.876 | 58.146 | 99.622 | 1.00 | 52.80 | C |
| ATOM | 14769 | C | PRO | D | 173 | 49.105 | 59.258 | 98.568 | 1.00 | 49.69 | C |
| ATOM | 14770 | O | PRO | D | 173 | 49.007 | 60.262 | 99.263 | 1.00 | 50.24 | O |
| ATOM | 14771 | N | ALA | D | 174 | 50.192 | 58.982 | 97.865 | 1.00 | 49.12 | N |
| ATOM | 14773 | CA | ALA | D | 174 | 51.333 | 59.885 | 97.868 | 1.00 | 48.07 | C |
| ATOM | 14775 | CB | ALA | D | 174 | 52.374 | 59.344 | 96.969 | 1.00 | 47.30 | C |
| ATOM | 14779 | C | ALA | D | 174 | 50.988 | 61.267 | 97.411 | 1.00 | 48.33 | C |
| ATOM | 14780 | O | ALA | D | 174 | 49.996 | 61.501 | 96.760 | 1.00 | 48.62 | O |
| ATOM | 14781 | N | VAL | D | 175 | 51.863 | 62.181 | 97.732 | 1.00 | 48.93 | N |
| ATOM | 14783 | CA | VAL | D | 175 | 51.693 | 63.544 | 97.311 | 1.00 | 50.08 | C |
| ATOM | 14785 | CB | VAL | D | 175 | 51.278 | 64.396 | 98.464 | 1.00 | 52.47 | C |
| ATOM | 14787 | CG1 | VAL | D | 175 | 51.091 | 65.789 | 98.004 | 1.00 | 56.48 | C |
| ATOM | 14791 | CG2 | VAL | D | 175 | 49.992 | 63.925 | 99.000 | 1.00 | 53.12 | C |
| ATOM | 14795 | C | VAL | D | 175 | 53.009 | 64.056 | 96.790 | 1.00 | 49.12 | C |
| ATOM | 14796 | O | VAL | D | 175 | 54.031 | 63.730 | 97.308 | 1.00 | 48.44 | O |
| ATOM | 14797 | N | LEU | D | 176 | 52.965 | 64.862 | 95.747 | 1.00 | 49.70 | N |
| ATOM | 14799 | CA | LEU | D | 176 | 54.162 | 65.349 | 95.125 | 1.00 | 49.52 | C |
| ATOM | 14801 | CB | LEU | D | 176 | 53.854 | 65.602 | 93.685 | 1.00 | 50.20 | C |
| ATOM | 14804 | CG | LEU | D | 176 | 54.982 | 66.015 | 92.758 | 1.00 | 51.28 | C |
| ATOM | 14806 | CD1 | LEU | D | 176 | 56.160 | 65.079 | 92.906 | 1.00 | 51.18 | C |
| ATOM | 14810 | CD2 | LEU | D | 176 | 54.408 | 65.949 | 91.360 | 1.00 | 51.54 | C |
| ATOM | 14814 | C | LEU | D | 176 | 54.528 | 66.631 | 95.771 | 1.00 | 51.36 | C |
| ATOM | 14815 | O | LEU | D | 176 | 53.750 | 67.559 | 95.800 | 1.00 | 53.28 | O |
| ATOM | 14816 | N | GLN | D | 177 | 55.703 | 66.664 | 96.348 | 1.00 | 52.30 | N |
| ATOM | 14818 | CA | GLN | D | 177 | 56.173 | 67.852 | 97.072 | 1.00 | 55.83 | C |
| ATOM | 14820 | CB | GLN | D | 177 | 57.242 | 67.468 | 98.109 | 1.00 | 56.07 | C |
| ATOM | 14823 | CG | GLN | D | 177 | 56.885 | 66.367 | 99.070 | 1.00 | 53.77 | C |
| ATOM | 14826 | CD | GLN | D | 177 | 58.093 | 65.861 | 99.821 | 1.00 | 55.31 | C |
| ATOM | 14827 | OE1 | GLN | D | 177 | 59.134 | 66.373 | 99.640 | 1.00 | 67.15 | O |
| ATOM | 14828 | NE2 | GLN | D | 177 | 57.940 | 64.937 | 100.701 | 1.00 | 56.54 | N |
| ATOM | 14831 | C | GLN | D | 177 | 56.753 | 68.909 | 96.092 | 1.00 | 57.83 | C |
| ATOM | 14832 | O | GLN | D | 177 | 56.895 | 68.652 | 94.905 | 1.00 | 57.07 | O |
| ATOM | 14833 | N | SER | D | 178 | 57.105 | 70.086 | 96.594 | 1.00 | 61.86 | N |
| ATOM | 14835 | CA | SER | D | 178 | 57.698 | 71.128 | 95.747 | 1.00 | 64.24 | C |
| ATOM | 14837 | CB | SER | D | 178 | 57.821 | 72.425 | 96.515 | 1.00 | 68.85 | C |
| ATOM | 14840 | OG | SER | D | 178 | 56.592 | 73.127 | 96.433 | 1.00 | 70.78 | O |
| ATOM | 14842 | C | SER | D | 178 | 59.054 | 70.760 | 95.178 | 1.00 | 63.76 | C |
| ATOM | 14843 | O | SER | D | 178 | 59.474 | 71.301 | 94.193 | 1.00 | 64.54 | O |
| ATOM | 14844 | N | SER | D | 179 | 59.735 | 69.827 | 95.820 | 1.00 | 63.67 | N |
| ATOM | 14846 | CA | SER | D | 179 | 61.005 | 69.276 | 95.324 | 1.00 | 63.08 | C |
| ATOM | 14848 | CB | SER | D | 179 | 61.691 | 68.545 | 96.476 | 1.00 | 63.84 | C |
| ATOM | 14851 | OG | SER | D | 179 | 60.849 | 67.484 | 97.003 | 1.00 | 62.76 | O |
| ATOM | 14853 | C | SER | D | 179 | 60.897 | 68.260 | 94.149 | 1.00 | 60.11 | C |
| ATOM | 14854 | O | SER | D | 179 | 61.972 | 67.795 | 93.639 | 1.00 | 60.04 | O |
| ATOM | 14855 | N | GLY | D | 180 | 59.651 | 67.856 | 93.788 | 1.00 | 56.93 | N |
| ATOM | 14857 | CA | GLY | D | 180 | 59.442 | 66.838 | 92.771 | 1.00 | 53.61 | C |
| ATOM | 14860 | C | GLY | D | 180 | 59.604 | 65.423 | 93.306 | 1.00 | 51.38 | C |
| ATOM | 14861 | O | GLY | D | 180 | 59.592 | 64.412 | 92.557 | 1.00 | 49.29 | O |
| ATOM | 14862 | N | LEU | D | 181 | 59.728 | 65.348 | 94.623 | 1.00 | 51.34 | N |
| ATOM | 14864 | CA | LEU | D | 181 | 59.769 | 64.068 | 95.288 | 1.00 | 50.39 | C |
| ATOM | 14866 | CB | LEU | D | 181 | 60.814 | 64.149 | 96.347 | 1.00 | 52.46 | C |
| ATOM | 14869 | CG | LEU | D | 181 | 62.218 | 64.303 | 95.845 | 1.00 | 53.18 | C |
| ATOM | 14871 | CD1 | LEU | D | 181 | 63.179 | 64.532 | 97.017 | 1.00 | 53.74 | C |
| ATOM | 14875 | CD2 | LEU | D | 181 | 62.516 | 63.006 | 95.073 | 1.00 | 51.26 | C |
| ATOM | 14879 | C | LEU | D | 181 | 58.437 | 63.756 | 95.964 | 1.00 | 49.42 | C |
| ATOM | 14880 | O | LEU | D | 181 | 57.823 | 64.632 | 96.590 | 1.00 | 50.84 | O |
| ATOM | 14881 | N | TYR | D | 182 | 57.995 | 62.517 | 95.866 | 1.00 | 47.37 | N |
| ATOM | 14883 | CA | TYR | D | 182 | 56.805 | 62.099 | 96.625 | 1.00 | 47.21 | C |
| ATOM | 14885 | CB | TYR | D | 182 | 56.283 | 60.780 | 96.092 | 1.00 | 46.17 | C |
| ATOM | 14888 | CG | TYR | D | 182 | 55.767 | 60.930 | 94.708 | 1.00 | 45.87 | C |
| ATOM | 14889 | CD1 | TYR | D | 182 | 56.557 | 60.615 | 93.633 | 1.00 | 46.82 | C |
| ATOM | 14891 | CE1 | TYR | D | 182 | 56.098 | 60.809 | 92.348 | 1.00 | 48.35 | C |
| ATOM | 14893 | CZ | TYR | D | 182 | 54.813 | 61.268 | 92.138 | 1.00 | 47.09 | C |
| ATOM | 14894 | OH | TYR | D | 182 | 54.417 | 61.435 | 90.868 | 1.00 | 45.62 | O |
| ATOM | 14896 | CE2 | TYR | D | 182 | 54.012 | 61.607 | 93.185 | 1.00 | 45.99 | C |
| ATOM | 14898 | CD2 | TYR | D | 182 | 54.494 | 61.461 | 94.465 | 1.00 | 46.47 | C |
| ATOM | 14900 | C | TYR | D | 182 | 57.045 | 61.914 | 98.120 | 1.00 | 48.05 | C |
| ATOM | 14901 | O | TYR | D | 182 | 58.117 | 61.576 | 98.545 | 1.00 | 48.45 | O |
| ATOM | 14902 | N | SER | D | 183 | 56.034 | 62.171 | 98.915 | 1.00 | 48.87 | N |
| ATOM | 14904 | CA | SER | D | 183 | 55.925 | 61.467 | 100.149 | 1.00 | 51.41 | C |
| ATOM | 14906 | CB | SER | D | 183 | 56.474 | 62.252 | 101.324 | 1.00 | 53.74 | C |
| ATOM | 14909 | OG | SER | D | 183 | 55.445 | 62.952 | 101.902 | 1.00 | 57.05 | O |
| ATOM | 14911 | C | SER | D | 183 | 54.518 | 60.978 | 100.430 | 1.00 | 51.30 | C |
| ATOM | 14912 | O | SER | D | 183 | 53.558 | 61.281 | 99.726 | 1.00 | 50.46 | O |
| ATOM | 14913 | N | LEU | D | 184 | 54.452 | 60.236 | 101.521 | 1.00 | 53.32 | N |
| ATOM | 14915 | CA | LEU | D | 184 | 53.409 | 59.260 | 101.805 | 1.00 | 54.25 | C |
| ATOM | 14917 | CB | LEU | D | 184 | 53.948 | 57.905 | 101.387 | 1.00 | 54.63 | C |
| ATOM | 14920 | CG | LEU | D | 184 | 53.018 | 56.728 | 101.250 | 1.00 | 54.80 | C |
| ATOM | 14922 | CD1 | LEU | D | 184 | 52.219 | 56.950 | 100.076 | 1.00 | 53.73 | C |
| ATOM | 14926 | CD2 | LEU | D | 184 | 53.879 | 55.526 | 101.028 | 1.00 | 57.84 | C |
| ATOM | 14930 | C | LEU | D | 184 | 53.275 | 59.190 | 103.308 | 1.00 | 56.22 | C |
| ATOM | 14931 | O | LEU | D | 184 | 54.094 | 59.680 | 104.021 | 1.00 | 57.29 | O |
| ATOM | 14932 | N | SER | D | 185 | 52.272 | 58.516 | 103.800 | 1.00 | 57.35 | N |
| ATOM | 14934 | CA | SER | D | 185 | 52.379 | 58.007 | 105.174 | 1.00 | 59.70 | C |
| ATOM | 14936 | CB | SER | D | 185 | 52.049 | 59.067 | 106.224 | 1.00 | 61.87 | C |
| ATOM | 14939 | OG | SER | D | 185 | 50.921 | 59.813 | 105.836 | 1.00 | 63.92 | O |
| ATOM | 14941 | C | SER | D | 185 | 51.474 | 56.865 | 105.319 | 1.00 | 58.54 | C |
| ATOM | 14942 | O | SER | D | 185 | 50.405 | 56.906 | 104.803 | 1.00 | 55.56 | O |
| ATOM | 14943 | N | SER | D | 186 | 51.955 | 55.837 | 105.977 | 1.00 | 60.18 | N |
| ATOM | 14945 | CA | SER | D | 186 | 51.078 | 54.790 | 106.428 | 1.00 | 63.53 | C |
| ATOM | 14947 | CB | SER | D | 186 | 51.565 | 53.439 | 105.983 | 1.00 | 64.38 | C |
| ATOM | 14950 | OG | SER | D | 186 | 51.306 | 52.536 | 107.024 | 1.00 | 69.79 | O |
| ATOM | 14952 | C | SER | D | 186 | 50.824 | 54.828 | 107.946 | 1.00 | 66.75 | C |
| ATOM | 14953 | O | SER | D | 186 | 51.600 | 55.365 | 108.716 | 1.00 | 68.12 | O |
| ATOM | 14954 | N | VAL | D | 187 | 49.700 | 54.263 | 108.364 | 1.00 | 69.09 | N |
| ATOM | 14956 | CA | VAL | D | 187 | 49.165 | 54.560 | 109.697 | 1.00 | 72.02 | C |
| ATOM | 14958 | CB | VAL | D | 187 | 48.374 | 55.912 | 109.640 | 1.00 | 70.96 | C |
| ATOM | 14960 | CG1 | VAL | D | 187 | 47.059 | 55.856 | 110.271 | 1.00 | 72.97 | C |
| ATOM | 14964 | CG2 | VAL | D | 187 | 49.184 | 57.018 | 110.284 | 1.00 | 72.15 | C |
| ATOM | 14968 | C | VAL | D | 187 | 48.375 | 53.380 | 110.180 | 1.00 | 74.57 | C |
| ATOM | 14969 | O | VAL | D | 187 | 47.765 | 52.687 | 109.388 | 1.00 | 73.67 | O |
| ATOM | 14970 | N | VAL | D | 188 | 48.480 | 53.089 | 111.467 | 1.00 | 78.78 | N |
| ATOM | 14972 | CA | VAL | D | 188 | 47.669 | 52.026 | 112.060 | 1.00 | 83.05 | C |
| ATOM | 14974 | CB | VAL | D | 188 | 48.424 | 50.763 | 112.506 | 1.00 | 86.25 | C |
| ATOM | 14976 | CG1 | VAL | D | 188 | 47.620 | 49.558 | 112.107 | 1.00 | 88.84 | C |
| ATOM | 14980 | CG2 | VAL | D | 188 | 49.766 | 50.672 | 111.881 | 1.00 | 86.59 | C |
| ATOM | 14984 | C | VAL | D | 188 | 46.954 | 52.550 | 113.272 | 1.00 | 86.01 | C |
| ATOM | 14985 | O | VAL | D | 188 | 47.492 | 53.373 | 114.027 | 1.00 | 87.50 | O |
| ATOM | 14986 | N | THR | D | 189 | 45.717 | 52.090 | 113.409 | 1.00 | 87.42 | N |
| ATOM | 14988 | CA | THR | D | 189 | 45.015 | 52.183 | 114.633 | 1.00 | 90.97 | C |
| ATOM | 14990 | CB | THR | D | 189 | 43.536 | 52.320 | 114.347 | 1.00 | 91.22 | C |
| ATOM | 14992 | OG1 | THR | D | 189 | 43.307 | 53.207 | 113.261 | 1.00 | 85.40 | O |
| ATOM | 14994 | CG2 | THR | D | 189 | 42.842 | 52.982 | 115.480 | 1.00 | 95.29 | C |
| ATOM | 14998 | C | THR | D | 189 | 45.331 | 50.835 | 115.304 | 1.00 | 95.78 | C |
| ATOM | 14999 | O | THR | D | 189 | 45.260 | 49.755 | 114.672 | 1.00 | 96.14 | O |
| ATOM | 15000 | N | VAL | D | 190 | 45.746 | 50.884 | 116.561 | 1.00 | 99.65 | N |
| ATOM | 15002 | CA | VAL | D | 190 | 45.807 | 49.661 | 117.344 | 1.00 | 105.40 | C |
| ATOM | 15004 | CB | VAL | D | 190 | 47.212 | 48.949 | 117.308 | 1.00 | 106.50 | C |
| ATOM | 15006 | CG1 | VAL | D | 190 | 47.112 | 47.459 | 117.879 | 1.00 | 113.44 | C |
| ATOM | 15010 | CG2 | VAL | D | 190 | 47.766 | 48.880 | 115.927 | 1.00 | 101.20 | C |
| ATOM | 15014 | C | VAL | D | 190 | 45.413 | 49.955 | 118.791 | 1.00 | 110.32 | C |
| ATOM | 15015 | O | VAL | D | 190 | 45.707 | 51.074 | 119.275 | 1.00 | 109.76 | O |
| ATOM | 15016 | N | PRO | D | 191 | 44.744 | 48.985 | 119.460 | 1.00 | 114.97 | N |
| ATOM | 15017 | CA | PRO | D | 191 | 44.542 | 49.062 | 120.915 | 1.00 | 120.91 | C |
| ATOM | 15019 | CB | PRO | D | 191 | 44.043 | 47.643 | 121.300 | 1.00 | 125.96 | C |
| ATOM | 15022 | CG | PRO | D | 191 | 44.151 | 46.807 | 120.045 | 1.00 | 123.19 | C |
| ATOM | 15025 | CD | PRO | D | 191 | 44.102 | 47.772 | 118.900 | 1.00 | 115.97 | C |
| ATOM | 15028 | C | PRO | D | 191 | 45.853 | 49.394 | 121.632 | 1.00 | 122.98 | C |
| ATOM | 15029 | O | PRO | D | 191 | 46.839 | 48.659 | 121.471 | 1.00 | 123.92 | O |
| ATOM | 15030 | N | SER | D | 192 | 45.865 | 50.488 | 122.396 | 1.00 | 124.39 | N |
| ATOM | 15032 | CA | SER | D | 192 | 47.084 | 50.948 | 123.089 | 1.00 | 126.95 | C |
| ATOM | 15034 | CB | SER | D | 192 | 46.777 | 52.111 | 124.075 | 1.00 | 129.91 | C |
| ATOM | 15037 | OG | SER | D | 192 | 45.532 | 51.972 | 124.740 | 1.00 | 133.29 | O |
| ATOM | 15039 | C | SER | D | 192 | 47.947 | 49.817 | 123.741 | 1.00 | 132.43 | C |
| ATOM | 15040 | O | SER | D | 192 | 49.151 | 49.762 | 123.488 | 1.00 | 131.87 | O |
| ATOM | 15041 | N | SER | D | 193 | 47.354 | 48.897 | 124.509 | 1.00 | 137.70 | N |
| ATOM | 15043 | CA | SER | D | 193 | 48.126 | 47.756 | 125.053 | 1.00 | 143.45 | C |
| ATOM | 15045 | CB | SER | D | 193 | 47.327 | 46.438 | 125.055 | 1.00 | 146.12 | C |
| ATOM | 15048 | OG | SER | D | 193 | 46.031 | 46.630 | 125.549 | 1.00 | 145.50 | O |
| ATOM | 15050 | C | SER | D | 193 | 49.469 | 47.537 | 124.314 | 1.00 | 141.77 | C |
| ATOM | 15051 | O | SER | D | 193 | 50.505 | 48.003 | 124.802 | 1.00 | 144.72 | O |
| ATOM | 15052 | N | SER | D | 194 | 49.431 | 46.911 | 123.120 | 1.00 | 137.58 | N |
| ATOM | 15054 | CA | SER | D | 194 | 50.576 | 46.172 | 122.505 | 1.00 | 137.39 | C |
| ATOM | 15056 | CB | SER | D | 194 | 51.823 | 47.067 | 122.336 | 1.00 | 134.79 | C |
| ATOM | 15059 | OG | SER | D | 194 | 51.599 | 48.394 | 122.757 | 1.00 | 130.77 | O |
| ATOM | 15061 | C | SER | D | 194 | 50.904 | 44.805 | 123.248 | 1.00 | 146.68 | C |
| ATOM | 15062 | O | SER | D | 194 | 52.066 | 44.543 | 123.660 | 1.00 | 150.94 | O |
| ATOM | 15063 | N | LEU | D | 195 | 49.851 | 43.974 | 123.443 | 1.00 | 150.16 | N |
| ATOM | 15065 | CA | LEU | D | 195 | 49.925 | 42.548 | 123.927 | 1.00 | 158.06 | C |
| ATOM | 15067 | CB | LEU | D | 195 | 48.727 | 42.204 | 124.827 | 1.00 | 162.41 | C |
| ATOM | 15073 | C | LEU | D | 195 | 49.952 | 41.592 | 122.701 | 1.00 | 156.29 | C |
| ATOM | 15074 | O | LEU | D | 195 | 49.549 | 42.026 | 121.607 | 1.00 | 149.95 | O |
| ATOM | 15075 | N | GLY | D | 196 | 50.425 | 40.340 | 122.842 | 1.00 | 162.68 | N |
| ATOM | 15077 | CA | GLY | D | 196 | 50.727 | 39.497 | 121.675 | 1.00 | 161.42 | C |
| ATOM | 15080 | C | GLY | D | 196 | 51.318 | 40.257 | 120.455 | 1.00 | 153.51 | C |
| ATOM | 15081 | O | GLY | D | 196 | 50.581 | 40.655 | 119.512 | 1.00 | 147.54 | O |
| ATOM | 15082 | N | THR | D | 197 | 52.642 | 40.478 | 120.464 | 1.00 | 153.86 | N |
| ATOM | 15084 | CA | THR | D | 197 | 53.368 | 41.350 | 119.480 | 1.00 | 146.39 | C |
| ATOM | 15086 | CB | THR | D | 197 | 53.254 | 40.820 | 117.993 | 1.00 | 142.49 | C |
| ATOM | 15088 | OG1 | THR | D | 197 | 54.045 | 39.617 | 117.882 | 1.00 | 149.06 | O |
| ATOM | 15090 | CG2 | THR | D | 197 | 53.887 | 41.806 | 116.926 | 1.00 | 133.52 | C |
| ATOM | 15094 | C | THR | D | 197 | 53.138 | 42.899 | 119.643 | 1.00 | 140.17 | C |
| ATOM | 15095 | O | THR | D | 197 | 52.525 | 43.607 | 118.818 | 1.00 | 132.05 | O |
| ATOM | 15096 | N | GLN | D | 198 | 53.709 | 43.363 | 120.759 | 1.00 | 144.11 | N |
| ATOM | 15098 | CA | GLN | D | 198 | 53.825 | 44.764 | 121.116 | 1.00 | 140.43 | C |
| ATOM | 15100 | CB | GLN | D | 198 | 54.450 | 44.898 | 122.532 | 1.00 | 147.96 | C |
| ATOM | 15107 | C | GLN | D | 198 | 54.697 | 45.496 | 120.114 | 1.00 | 134.16 | C |
| ATOM | 15108 | O | GLN | D | 198 | 54.789 | 46.710 | 120.188 | 1.00 | 130.78 | O |
| ATOM | 15109 | N | THR | D | 199 | 55.332 | 44.766 | 119.190 | 1.00 | 133.07 | N |
| ATOM | 15111 | CA | THR | D | 199 | 56.296 | 45.351 | 118.244 | 1.00 | 128.45 | C |
| ATOM | 15113 | CB | THR | D | 199 | 57.444 | 44.346 | 117.891 | 1.00 | 132.44 | C |
| ATOM | 15115 | OG1 | THR | D | 199 | 57.295 | 43.134 | 118.631 | 1.00 | 139.60 | O |
| ATOM | 15117 | CG2 | THR | D | 199 | 58.814 | 44.873 | 118.344 | 1.00 | 135.37 | C |
| ATOM | 15121 | C | THR | D | 199 | 55.626 | 45.857 | 116.943 | 1.00 | 120.09 | C |
| ATOM | 15122 | O | THR | D | 199 | 55.024 | 45.084 | 116.183 | 1.00 | 118.92 | O |
| ATOM | 15123 | N | TYR | D | 200 | 55.751 | 47.155 | 116.680 | 1.00 | 115.03 | N |
| ATOM | 15125 | CA | TYR | D | 200 | 55.213 | 47.730 | 115.454 | 1.00 | 107.62 | C |
| ATOM | 15127 | CB | TYR | D | 200 | 54.281 | 48.908 | 115.795 | 1.00 | 104.46 | C |
| ATOM | 15130 | CG | TYR | D | 200 | 53.147 | 48.489 | 116.711 | 1.00 | 106.68 | C |
| ATOM | 15131 | CD1 | TYR | D | 200 | 52.975 | 49.070 | 117.970 | 1.00 | 109.12 | C |
| ATOM | 15133 | CE1 | TYR | D | 200 | 51.963 | 48.664 | 118.811 | 1.00 | 111.35 | C |
| ATOM | 15135 | CZ | TYR | D | 200 | 51.115 | 47.661 | 118.401 | 1.00 | 111.91 | C |
| ATOM | 15136 | OH | TYR | D | 200 | 50.096 | 47.221 | 119.207 | 1.00 | 115.71 | O |
| ATOM | 15138 | CE2 | TYR | D | 200 | 51.271 | 47.068 | 117.166 | 1.00 | 109.67 | C |
| ATOM | 15140 | CD2 | TYR | D | 200 | 52.277 | 47.479 | 116.333 | 1.00 | 106.06 | C |
| ATOM | 15142 | C | TYR | D | 200 | 56.346 | 48.132 | 114.499 | 1.00 | 104.92 | C |
| ATOM | 15143 | O | TYR | D | 200 | 57.050 | 49.105 | 114.738 | 1.00 | 104.47 | O |
| ATOM | 15144 | N | ILE | D | 201 | 56.521 | 47.366 | 113.424 | 1.00 | 103.81 | N |
| ATOM | 15146 | CA | ILE | D | 201 | 57.545 | 47.671 | 112.429 | 1.00 | 102.21 | C |
| ATOM | 15148 | CB | ILE | D | 201 | 58.684 | 46.590 | 112.496 | 1.00 | 107.75 | C |
| ATOM | 15150 | CG1 | ILE | D | 201 | 59.393 | 46.656 | 113.856 | 1.00 | 113.72 | C |
| ATOM | 15153 | CD1 | ILE | D | 201 | 60.523 | 45.656 | 114.048 | 1.00 | 119.86 | C |
| ATOM | 15157 | CG2 | ILE | D | 201 | 59.717 | 46.792 | 111.336 | 1.00 | 105.63 | C |
| ATOM | 15161 | C | ILE | D | 201 | 56.997 | 47.807 | 110.980 | 1.00 | 96.83 | C |
| ATOM | 15162 | O | ILE | D | 201 | 56.436 | 46.849 | 110.436 | 1.00 | 97.98 | O |
| ATOM | 15163 | N | CYS | D | 202 | 57.184 | 48.959 | 110.330 | 1.00 | 92.25 | N |
| ATOM | 15165 | CA | CYS | D | 202 | 56.695 | 49.076 | 108.944 | 1.00 | 87.63 | C |
| ATOM | 15167 | CB | CYS | D | 202 | 56.006 | 50.398 | 108.708 | 1.00 | 83.22 | C |
| ATOM | 15170 | SG | CYS | D | 202 | 57.133 | 51.729 | 108.422 | 1.00 | 81.91 | S |
| ATOM | 15171 | C | CYS | D | 202 | 57.728 | 48.846 | 107.836 | 1.00 | 86.17 | C |
| ATOM | 15172 | O | CYS | D | 202 | 58.868 | 49.308 | 107.923 | 1.00 | 86.56 | O |
| ATOM | 15173 | N | ASN | D | 203 | 57.254 | 48.177 | 106.780 | 1.00 | 84.31 | N |
| ATOM | 15175 | CA | ASN | D | 203 | 58.079 | 47.615 | 105.732 | 1.00 | 84.05 | C |
| ATOM | 15177 | CB | ASN | D | 203 | 57.692 | 46.171 | 105.447 | 1.00 | 87.34 | C |
| ATOM | 15180 | CG | ASN | D | 203 | 57.400 | 45.402 | 106.715 | 1.00 | 93.02 | C |
| ATOM | 15181 | OD1 | ASN | D | 203 | 58.163 | 45.443 | 107.705 | 1.00 | 94.19 | O |
| ATOM | 15182 | ND2 | ASN | D | 203 | 56.273 | 44.708 | 106.709 | 1.00 | 95.69 | N |
| ATOM | 15185 | C | ASN | D | 203 | 57.828 | 48.450 | 104.543 | 1.00 | 78.21 | C |
| ATOM | 15186 | O | ASN | D | 203 | 56.798 | 48.349 | 103.918 | 1.00 | 77.07 | O |
| ATOM | 15187 | N | VAL | D | 204 | 58.774 | 49.310 | 104.262 | 1.00 | 75.80 | N |
| ATOM | 15189 | CA | VAL | D | 204 | 58.728 | 50.161 | 103.109 | 1.00 | 71.12 | C |
| ATOM | 15191 | CB | VAL | D | 204 | 59.273 | 51.551 | 103.467 | 1.00 | 69.36 | C |
| ATOM | 15193 | CG1 | VAL | D | 204 | 58.892 | 52.562 | 102.404 | 1.00 | 64.60 | C |
| ATOM | 15197 | CG2 | VAL | D | 204 | 58.753 | 51.967 | 104.837 | 1.00 | 69.88 | C |
| ATOM | 15201 | C | VAL | D | 204 | 59.544 | 49.533 | 101.980 | 1.00 | 71.71 | C |
| ATOM | 15202 | O | VAL | D | 204 | 60.760 | 49.405 | 102.076 | 1.00 | 73.45 | O |
| ATOM | 15203 | N | ASN | D | 205 | 58.867 | 49.133 | 100.912 | 1.00 | 70.66 | N |
| ATOM | 15205 | CA | ASN | D | 205 | 59.554 | 48.715 | 99.697 | 1.00 | 71.49 | C |
| ATOM | 15207 | CB | ASN | D | 205 | 59.047 | 47.351 | 99.229 | 1.00 | 74.94 | C |
| ATOM | 15210 | CG | ASN | D | 205 | 59.923 | 46.761 | 98.143 | 1.00 | 78.41 | C |
| ATOM | 15211 | OD1 | ASN | D | 205 | 60.868 | 46.022 | 98.437 | 1.00 | 84.36 | O |
| ATOM | 15212 | ND2 | ASN | D | 205 | 59.661 | 47.141 | 96.874 | 1.00 | 77.11 | N |
| ATOM | 15215 | C | ASN | D | 205 | 59.436 | 49.745 | 98.564 | 1.00 | 66.99 | C |
| ATOM | 15216 | O | ASN | D | 205 | 58.335 | 50.165 | 98.188 | 1.00 | 65.08 | O |
| ATOM | 15217 | N | HIS | D | 206 | 60.575 | 50.160 | 98.031 | 1.00 | 66.16 | N |
| ATOM | 15219 | CA | HIS | D | 206 | 60.573 | 51.013 | 96.867 | 1.00 | 63.53 | C |
| ATOM | 15221 | CB | HIS | D | 206 | 61.317 | 52.331 | 97.165 | 1.00 | 62.70 | C |
| ATOM | 15224 | CG | HIS | D | 206 | 61.168 | 53.375 | 96.096 | 1.00 | 59.74 | C |
| ATOM | 15225 | ND1 | HIS | D | 206 | 62.041 | 53.481 | 95.038 | 1.00 | 60.76 | N |
| ATOM | 15227 | CE1 | HIS | D | 206 | 61.660 | 54.482 | 94.258 | 1.00 | 60.76 | C |
| ATOM | 15229 | NE2 | HIS | D | 206 | 60.570 | 55.028 | 94.772 | 1.00 | 56.00 | N |
| ATOM | 15231 | CD2 | HIS | D | 206 | 60.261 | 54.371 | 95.937 | 1.00 | 57.75 | C |
| ATOM | 15233 | C | HIS | D | 206 | 61.213 | 50.257 | 95.718 | 1.00 | 65.17 | C |
| ATOM | 15234 | O | HIS | D | 206 | 62.419 | 50.373 | 95.513 | 1.00 | 66.55 | O |
| ATOM | 15235 | N | LYS | D | 207 | 60.399 | 49.504 | 94.963 | 1.00 | 65.74 | N |
| ATOM | 15237 | CA | LYS | D | 207 | 60.878 | 48.605 | 93.893 | 1.00 | 67.90 | C |
| ATOM | 15239 | CB | LYS | D | 207 | 59.738 | 47.730 | 93.323 | 1.00 | 69.51 | C |
| ATOM | 15246 | C | LYS | D | 207 | 61.623 | 49.304 | 92.746 | 1.00 | 65.87 | C |
| ATOM | 15247 | O | LYS | D | 207 | 62.591 | 48.740 | 92.218 | 1.00 | 69.15 | O |
| ATOM | 15248 | N | PRO | D | 208 | 61.243 | 50.518 | 92.351 | 1.00 | 61.80 | N |
| ATOM | 15249 | CA | PRO | D | 208 | 61.943 | 51.167 | 91.206 | 1.00 | 60.92 | C |
| ATOM | 15251 | CB | PRO | D | 208 | 61.030 | 52.336 | 90.824 | 1.00 | 56.74 | C |
| ATOM | 15254 | CG | PRO | D | 208 | 60.162 | 52.553 | 91.983 | 1.00 | 55.16 | C |
| ATOM | 15257 | CD | PRO | D | 208 | 60.202 | 51.389 | 92.929 | 1.00 | 58.46 | C |
| ATOM | 15260 | C | PRO | D | 208 | 63.412 | 51.630 | 91.492 | 1.00 | 61.31 | C |
| ATOM | 15261 | O | PRO | D | 208 | 64.183 | 51.824 | 90.547 | 1.00 | 61.70 | O |
| ATOM | 15262 | N | SER | D | 209 | 63.764 | 51.760 | 92.771 | 1.00 | 61.75 | N |
| ATOM | 15264 | CA | SER | D | 209 | 65.107 | 52.100 | 93.230 | 1.00 | 63.81 | C |
| ATOM | 15266 | CB | SER | D | 209 | 65.067 | 53.338 | 94.118 | 1.00 | 61.67 | C |
| ATOM | 15269 | OG | SER | D | 209 | 64.729 | 53.003 | 95.435 | 1.00 | 61.97 | O |
| ATOM | 15271 | C | SER | D | 209 | 65.706 | 51.015 | 94.078 | 1.00 | 68.74 | C |
| ATOM | 15272 | O | SER | D | 209 | 66.705 | 51.256 | 94.740 | 1.00 | 71.24 | O |
| ATOM | 15273 | N | ASN | D | 210 | 65.085 | 49.836 | 94.113 | 1.00 | 70.96 | N |
| ATOM | 15275 | CA | ASN | D | 210 | 65.640 | 48.698 | 94.851 | 1.00 | 75.73 | C |
| ATOM | 15277 | CB | ASN | D | 210 | 67.009 | 48.338 | 94.275 | 1.00 | 79.41 | C |
| ATOM | 15280 | CG | ASN | D | 210 | 67.493 | 47.009 | 94.735 | 1.00 | 85.45 | C |
| ATOM | 15281 | OD1 | ASN | D | 210 | 66.693 | 46.147 | 95.042 | 1.00 | 86.85 | O |
| ATOM | 15282 | ND2 | ASN | D | 210 | 68.826 | 46.840 | 94.825 | 1.00 | 91.76 | N |
| ATOM | 15285 | C | ASN | D | 210 | 65.746 | 48.906 | 96.367 | 1.00 | 76.54 | C |
| ATOM | 15286 | O | ASN | D | 210 | 66.441 | 48.163 | 97.027 | 1.00 | 81.20 | O |
| ATOM | 15287 | N | THR | D | 211 | 65.043 | 49.891 | 96.915 | 1.00 | 73.00 | N |
| ATOM | 15289 | CA | THR | D | 211 | 65.020 | 50.089 | 98.360 | 1.00 | 75.11 | C |
| ATOM | 15291 | CB | THR | D | 211 | 64.668 | 51.546 | 98.732 | 1.00 | 71.89 | C |
| ATOM | 15293 | OG1 | THR | D | 211 | 64.896 | 52.438 | 97.628 | 1.00 | 70.10 | O |
| ATOM | 15295 | CG2 | THR | D | 211 | 65.666 | 52.070 | 99.782 | 1.00 | 76.24 | C |
| ATOM | 15299 | C | THR | D | 211 | 64.044 | 49.148 | 99.040 | 1.00 | 76.40 | C |
| ATOM | 15300 | O | THR | D | 211 | 62.956 | 48.902 | 98.516 | 1.00 | 74.76 | O |
| ATOM | 15301 | N | LYS | D | 212 | 64.432 | 48.605 | 100.193 | 1.00 | 80.57 | N |
| ATOM | 15303 | CA | LYS | D | 212 | 63.545 | 47.720 | 100.946 | 1.00 | 82.72 | C |
| ATOM | 15305 | CB | LYS | D | 212 | 63.730 | 46.229 | 100.569 | 1.00 | 87.28 | C |
| ATOM | 15312 | C | LYS | D | 212 | 63.809 | 47.921 | 102.405 | 1.00 | 85.46 | C |
| ATOM | 15313 | O | LYS | D | 212 | 64.258 | 47.015 | 103.062 | 1.00 | 91.69 | O |
| ATOM | 15314 | N | VAL | D | 213 | 63.489 | 49.105 | 102.900 | 1.00 | 82.31 | N |
| ATOM | 15316 | CA | VAL | D | 213 | 63.725 | 49.519 | 104.291 | 1.00 | 85.17 | C |
| ATOM | 15318 | CB | VAL | D | 213 | 63.756 | 51.057 | 104.298 | 1.00 | 82.29 | C |
| ATOM | 15320 | CG1 | VAL | D | 213 | 63.512 | 51.666 | 105.713 | 1.00 | 84.73 | C |
| ATOM | 15324 | CG2 | VAL | D | 213 | 65.083 | 51.527 | 103.696 | 1.00 | 84.19 | C |
| ATOM | 15328 | C | VAL | D | 213 | 62.711 | 49.064 | 105.372 | 1.00 | 86.25 | C |
| ATOM | 15329 | O | VAL | D | 213 | 61.532 | 49.006 | 105.129 | 1.00 | 82.68 | O |
| ATOM | 15330 | N | ASP | D | 214 | 63.192 | 48.774 | 106.586 | 1.00 | 91.59 | N |
| ATOM | 15332 | CA | ASP | D | 214 | 62.311 | 48.532 | 107.749 | 1.00 | 92.98 | C |
| ATOM | 15334 | CB | ASP | D | 214 | 62.506 | 47.092 | 108.271 | 1.00 | 98.93 | C |
| ATOM | 15337 | CG | ASP | D | 214 | 62.194 | 46.035 | 107.204 | 1.00 | 97.80 | C |
| ATOM | 15338 | OD1 | ASP | D | 214 | 61.385 | 46.349 | 106.310 | 1.00 | 92.43 | O |
| ATOM | 15339 | OD2 | ASP | D | 214 | 62.686 | 44.884 | 107.157 | 1.00 | 100.35 | O |
| ATOM | 15340 | C | ASP | D | 214 | 62.580 | 49.592 | 108.846 | 1.00 | 94.11 | C |
| ATOM | 15341 | O | ASP | D | 214 | 63.738 | 50.011 | 109.018 | 1.00 | 97.04 | O |
| ATOM | 15342 | N | LYS | D | 215 | 61.519 | 50.079 | 109.507 | 1.00 | 91.96 | N |
| ATOM | 15344 | CA | LYS | D | 215 | 61.627 | 50.915 | 110.722 | 1.00 | 94.21 | C |
| ATOM | 15346 | CB | LYS | D | 215 | 61.440 | 52.411 | 110.394 | 1.00 | 88.82 | C |
| ATOM | 15353 | C | LYS | D | 215 | 60.643 | 50.460 | 111.849 | 1.00 | 96.87 | C |
| ATOM | 15354 | O | LYS | D | 215 | 59.545 | 50.002 | 111.586 | 1.00 | 94.60 | O |
| ATOM | 15355 | N | ARG | D | 216 | 61.060 | 50.595 | 113.105 | 1.00 | 102.63 | N |
| ATOM | 15357 | CA | ARG | D | 216 | 60.210 | 50.347 | 114.279 | 1.00 | 105.91 | C |
| ATOM | 15359 | CB | ARG | D | 216 | 61.002 | 49.578 | 115.347 | 1.00 | 114.01 | C |
| ATOM | 15368 | C | ARG | D | 216 | 59.655 | 51.654 | 114.900 | 1.00 | 104.92 | C |
| ATOM | 15369 | O | ARG | D | 216 | 60.344 | 52.686 | 114.955 | 1.00 | 104.73 | O |
| ATOM | 15370 | N | VAL | D | 217 | 58.416 | 51.599 | 115.392 | 1.00 | 104.77 | N |
| ATOM | 15372 | CA | VAL | D | 217 | 57.769 | 52.766 | 116.007 | 1.00 | 104.59 | C |
| ATOM | 15374 | CB | VAL | D | 217 | 56.461 | 53.206 | 115.224 | 1.00 | 98.42 | C |
| ATOM | 15376 | CG1 | VAL | D | 217 | 55.939 | 54.582 | 115.703 | 1.00 | 97.76 | C |
| ATOM | 15380 | CG2 | VAL | D | 217 | 56.697 | 53.270 | 113.726 | 1.00 | 92.68 | C |
| ATOM | 15384 | C | VAL | D | 217 | 57.468 | 52.519 | 117.502 | 1.00 | 111.16 | C |
| ATOM | 15385 | O | VAL | D | 217 | 56.318 | 52.305 | 117.890 | 1.00 | 111.38 | O |
| ATOM | 15386 | N | GLU | D | 218 | 58.506 | 52.549 | 118.337 | 1.00 | 117.44 | N |
| ATOM | 15388 | CA | GLU | D | 218 | 58.321 | 52.507 | 119.788 | 1.00 | 124.37 | C |
| ATOM | 15390 | CB | GLU | D | 218 | 59.670 | 52.285 | 120.523 | 1.00 | 132.09 | C |
| ATOM | 15393 | CG | GLU | D | 218 | 60.454 | 51.010 | 120.158 | 1.00 | 134.04 | C |
| ATOM | 15396 | CD | GLU | D | 218 | 61.880 | 51.285 | 119.658 | 1.00 | 134.59 | C |
| ATOM | 15397 | OE1 | GLU | D | 218 | 62.817 | 51.026 | 120.429 | 1.00 | 140.64 | O |
| ATOM | 15398 | OE2 | GLU | D | 218 | 62.079 | 51.756 | 118.499 | 1.00 | 128.11 | O |
| ATOM | 15399 | C | GLU | D | 218 | 57.741 | 53.874 | 120.171 | 1.00 | 123.49 | C |
| ATOM | 15400 | O | GLU | D | 218 | 57.926 | 54.837 | 119.434 | 1.00 | 119.12 | O |
| ATOM | 15401 | N | PRO | D | 219 | 57.050 | 53.982 | 121.306 | 1.00 | 128.14 | N |
| ATOM | 15402 | CA | PRO | D | 219 | 56.704 | 55.312 | 121.852 | 1.00 | 129.40 | C |
| ATOM | 15404 | CB | PRO | D | 219 | 55.714 | 54.997 | 122.995 | 1.00 | 133.43 | C |
| ATOM | 15407 | CG | PRO | D | 219 | 55.987 | 53.558 | 123.371 | 1.00 | 137.50 | C |
| ATOM | 15410 | CD | PRO | D | 219 | 56.555 | 52.876 | 122.145 | 1.00 | 132.81 | C |
| ATOM | 15413 | C | PRO | D | 219 | 57.965 | 56.083 | 122.345 | 1.00 | 134.98 | C |
| ATOM | 15414 | O | PRO | D | 219 | 59.093 | 55.751 | 121.941 | 1.00 | 136.16 | O |
| ATOM | 15415 | N | LYS | D | 220 | 57.773 | 57.114 | 123.171 | 1.00 | 138.92 | N |
| ATOM | 15417 | CA | LYS | D | 220 | 58.896 | 57.870 | 123.769 | 1.00 | 144.98 | C |
| ATOM | 15419 | CB | LYS | D | 220 | 59.535 | 58.834 | 122.740 | 1.00 | 140.79 | C |
| ATOM | 15426 | C | LYS | D | 220 | 58.390 | 58.627 | 125.010 | 1.00 | 151.08 | C |
| ATOM | 15427 | O | LYS | D | 220 | 57.270 | 59.161 | 124.935 | 1.00 | 147.70 | O |
| ATOM | 15428 | OXT | LYS | D | 220 | 59.055 | 58.684 | 126.069 | 1.00 | 159.07 | O |
| ATOM | 15429 | C1 | NAG | E | 321 | 29.462 | −7.124 | 22.592 | 1.00 | 106.85 | C |
| ATOM | 15432 | C2 | NAG | E | 321 | 28.616 | −8.080 | 23.433 | 1.00 | 108.34 | C |
| ATOM | 15434 | N2 | NAG | E | 321 | 27.778 | −7.288 | 24.383 | 1.00 | 103.56 | N |
| ATOM | 15436 | C7 | NAG | E | 321 | 26.642 | −6.621 | 24.066 | 1.00 | 100.08 | C |
| ATOM | 15437 | O7 | NAG | E | 321 | 25.925 | −6.934 | 23.152 | 1.00 | 101.12 | O |
| ATOM | 15438 | C8 | NAG | E | 321 | 26.201 | −5.421 | 24.839 | 1.00 | 96.46 | C |
| ATOM | 15442 | C3 | NAG | E | 321 | 29.493 | −9.202 | 24.102 | 1.00 | 112.92 | C |
| ATOM | 15444 | O3 | NAG | E | 321 | 28.990 | −10.588 | 23.960 | 1.00 | 118.55 | O |
| ATOM | 15446 | C4 | NAG | E | 321 | 31.051 | −9.069 | 23.931 | 1.00 | 113.06 | C |
| ATOM | 15448 | O4 | NAG | E | 321 | 31.842 | −9.641 | 24.956 | 1.00 | 113.90 | O |
| ATOM | 15450 | C5 | NAG | E | 321 | 31.401 | −7.591 | 23.904 | 1.00 | 108.70 | C |
| ATOM | 15452 | C6 | NAG | E | 321 | 32.887 | −7.238 | 24.154 | 1.00 | 106.46 | C |
| ATOM | 15455 | O6 | NAG | E | 321 | 33.769 | −8.115 | 23.495 | 1.00 | 106.75 | O |
| ATOM | 15457 | O5 | NAG | E | 321 | 30.883 | −7.285 | 22.627 | 1.00 | 108.92 | O |
| ATOM | 15458 | C1 | NAG | E | 322 | 29.267 | −9.662 | 26.756 | 1.00 | 110.31 | C |
| ATOM | 15461 | C2 | NAG | E | 322 | 28.521 | −10.967 | 26.426 | 1.00 | 114.38 | C |
| ATOM | 15463 | N2 | NAG | E | 322 | 28.052 | −10.913 | 25.020 | 1.00 | 116.92 | N |
| ATOM | 15465 | C7 | NAG | E | 322 | 28.385 | −11.780 | 24.057 | 1.00 | 119.42 | C |
| ATOM | 15466 | O7 | NAG | E | 322 | 29.320 | −11.475 | 23.340 | 1.00 | 121.28 | O |
| ATOM | 15467 | C8 | NAG | E | 322 | 27.607 | −13.054 | 23.839 | 1.00 | 120.01 | C |
| ATOM | 15471 | C3 | NAG | E | 322 | 27.408 | −11.226 | 27.470 | 1.00 | 113.49 | C |
| ATOM | 15473 | O3 | NAG | E | 322 | 26.898 | −12.548 | 27.431 | 1.00 | 115.37 | O |
| ATOM | 15475 | C4 | NAG | E | 322 | 27.936 | −10.952 | 28.884 | 1.00 | 112.48 | C |
| ATOM | 15477 | O4 | NAG | E | 322 | 26.846 | −11.144 | 29.763 | 1.00 | 111.54 | O |
| ATOM | 15479 | C5 | NAG | E | 322 | 28.645 | −9.568 | 28.956 | 1.00 | 109.09 | C |
| ATOM | 15481 | C6 | NAG | E | 322 | 29.263 | −9.135 | 30.280 | 1.00 | 107.46 | C |
| ATOM | 15484 | O6 | NAG | E | 322 | 28.277 | −8.551 | 31.092 | 1.00 | 105.99 | O |
| ATOM | 15486 | O5 | NAG | E | 322 | 29.744 | −9.611 | 28.071 | 1.00 | 109.51 | O |
| ATOM | 15487 | C1 | MAN | E | 323 | 27.558 | −13.638 | 31.168 | 1.00 | 118.90 | C |
| ATOM | 15490 | C2 | MAN | E | 323 | 26.564 | −13.183 | 32.294 | 1.00 | 117.77 | C |
| ATOM | 15492 | O2 | MAN | E | 323 | 26.868 | −11.917 | 32.907 | 1.00 | 116.43 | O |
| ATOM | 15494 | C3 | MAN | E | 323 | 25.081 | −13.205 | 31.860 | 1.00 | 117.73 | C |
| ATOM | 15496 | O3 | MAN | E | 323 | 24.400 | −11.977 | 32.023 | 1.00 | 112.67 | O |
| ATOM | 15498 | C4 | MAN | E | 323 | 24.976 | −13.765 | 30.434 | 1.00 | 119.97 | C |
| ATOM | 15500 | O4 | MAN | E | 323 | 23.637 | −13.872 | 30.008 | 1.00 | 121.73 | O |
| ATOM | 15502 | C5 | MAN | E | 323 | 25.750 | −15.104 | 30.461 | 1.00 | 122.58 | C |
| ATOM | 15504 | C6 | MAN | E | 323 | 25.209 | −16.307 | 29.627 | 1.00 | 125.63 | C |
| ATOM | 15507 | O6 | MAN | E | 323 | 24.816 | −16.087 | 28.285 | 1.00 | 125.42 | O |
| ATOM | 15509 | O5 | MAN | E | 323 | 27.124 | −14.727 | 30.312 | 1.00 | 121.55 | O |
| ATOM | 15510 | C1 | MAN | E | 324 | 25.902 | −11.845 | 35.755 | 1.00 | 115.15 | C |
| ATOM | 15513 | C2 | MAN | E | 324 | 27.196 | −11.304 | 35.140 | 1.00 | 112.82 | C |
| ATOM | 15515 | O2 | MAN | E | 324 | 26.996 | −11.188 | 33.736 | 1.00 | 113.36 | O |
| ATOM | 15517 | C3 | MAN | E | 324 | 27.691 | −9.982 | 35.758 | 1.00 | 109.46 | C |
| ATOM | 15519 | O3 | MAN | E | 324 | 28.466 | −9.354 | 34.763 | 1.00 | 107.10 | O |
| ATOM | 15521 | C4 | MAN | E | 324 | 26.596 | −8.988 | 36.246 | 1.00 | 108.78 | C |
| ATOM | 15523 | O4 | MAN | E | 324 | 27.023 | −8.082 | 37.287 | 1.00 | 103.92 | O |
| ATOM | 15525 | C5 | MAN | E | 324 | 25.275 | −9.715 | 36.643 | 1.00 | 112.59 | C |
| ATOM | 15527 | C6 | MAN | E | 324 | 24.102 | −8.722 | 36.842 | 1.00 | 110.06 | C |
| ATOM | 15530 | O6 | MAN | E | 324 | 23.750 | −8.085 | 35.622 | 1.00 | 108.11 | O |
| ATOM | 15532 | O5 | MAN | E | 324 | 24.926 | −10.812 | 35.755 | 1.00 | 114.60 | O |
| ATOM | 15533 | C1 | NAG | E | 881 | 51.524 | 67.218 | 74.770 | 1.00 | 82.89 | C |
| ATOM | 15536 | C2 | NAG | E | 881 | 50.338 | 68.025 | 74.160 | 1.00 | 83.13 | C |
| ATOM | 15538 | N2 | NAG | E | 881 | 49.032 | 67.353 | 73.995 | 1.00 | 82.71 | N |
| ATOM | 15540 | C7 | NAG | E | 881 | 47.991 | 67.386 | 74.866 | 1.00 | 82.73 | C |
| ATOM | 15541 | O7 | NAG | E | 881 | 47.380 | 66.346 | 75.237 | 1.00 | 78.34 | O |
| ATOM | 15542 | C8 | NAG | E | 881 | 47.603 | 68.758 | 75.385 | 1.00 | 84.08 | C |
| ATOM | 15546 | C3 | NAG | E | 881 | 50.819 | 68.571 | 72.803 | 1.00 | 83.44 | C |
| ATOM | 15548 | O3 | NAG | E | 881 | 49.657 | 69.047 | 72.125 | 1.00 | 81.68 | O |
| ATOM | 15550 | C4 | NAG | E | 881 | 52.075 | 69.513 | 72.964 | 1.00 | 84.89 | C |
| ATOM | 15552 | O4 | NAG | E | 881 | 52.660 | 69.876 | 71.736 | 1.00 | 84.61 | O |
| ATOM | 15554 | C5 | NAG | E | 881 | 53.218 | 68.848 | 73.783 | 1.00 | 83.76 | C |
| ATOM | 15556 | C6 | NAG | E | 881 | 54.566 | 69.652 | 74.031 | 1.00 | 85.43 | C |
| ATOM | 15559 | O6 | NAG | E | 881 | 54.704 | 71.055 | 74.263 | 1.00 | 83.52 | O |
| ATOM | 15561 | O5 | NAG | E | 881 | 52.665 | 68.147 | 74.940 | 1.00 | 86.69 | O |
| ATOM | 15562 | C1 | NAG | E | 3281 | 49.375 | 31.629 | 48.905 | 1.00 | 52.84 | C |
| ATOM | 15565 | C2 | NAG | E | 3281 | 49.482 | 31.018 | 50.297 | 1.00 | 51.74 | C |
| ATOM | 15567 | N2 | NAG | E | 3281 | 50.091 | 31.941 | 51.249 | 1.00 | 51.34 | N |
| ATOM | 15569 | C7 | NAG | E | 3281 | 49.384 | 32.526 | 52.211 | 1.00 | 50.13 | C |
| ATOM | 15570 | O7 | NAG | E | 3281 | 48.435 | 32.032 | 52.749 | 1.00 | 53.22 | O |
| ATOM | 15571 | C8 | NAG | E | 3281 | 49.756 | 33.856 | 52.704 | 1.00 | 50.66 | C |
| ATOM | 15575 | C3 | NAG | E | 3281 | 50.226 | 29.684 | 50.255 | 1.00 | 52.62 | C |
| ATOM | 15577 | O3 | NAG | E | 3281 | 50.116 | 29.054 | 51.488 | 1.00 | 52.27 | O |
| ATOM | 15579 | C4 | NAG | E | 3281 | 49.732 | 28.723 | 49.174 | 1.00 | 54.50 | C |
| ATOM | 15581 | O4 | NAG | E | 3281 | 50.703 | 27.716 | 48.931 | 1.00 | 54.62 | O |
| ATOM | 15582 | C5 | NAG | E | 3281 | 49.375 | 29.441 | 47.856 | 1.00 | 54.70 | C |
| ATOM | 15584 | C6 | NAG | E | 3281 | 48.447 | 28.552 | 47.010 | 1.00 | 55.71 | C |
| ATOM | 15587 | O6 | NAG | E | 3281 | 47.278 | 28.100 | 47.717 | 1.00 | 53.23 | O |
| ATOM | 15589 | O5 | NAG | E | 3281 | 48.735 | 30.686 | 48.078 | 1.00 | 51.91 | O |
| ATOM | 15590 | C1 | NAG | E | 3282 | 50.357 | 26.487 | 49.573 | 1.00 | 54.61 | C |
| ATOM | 15592 | C2 | NAG | E | 3282 | 51.035 | 25.314 | 48.888 | 1.00 | 56.44 | C |
| ATOM | 15594 | N2 | NAG | E | 3282 | 50.443 | 25.157 | 47.600 | 1.00 | 56.42 | N |
| ATOM | 15596 | C7 | NAG | E | 3282 | 50.897 | 25.560 | 46.412 | 1.00 | 57.36 | C |
| ATOM | 15597 | O7 | NAG | E | 3282 | 51.890 | 26.176 | 46.141 | 1.00 | 58.44 | O |
| ATOM | 15598 | C8 | NAG | E | 3282 | 50.036 | 25.176 | 45.269 | 1.00 | 58.52 | C |
| ATOM | 15602 | C3 | NAG | E | 3282 | 50.703 | 24.044 | 49.639 | 1.00 | 56.65 | C |
| ATOM | 15604 | O3 | NAG | E | 3282 | 51.439 | 22.899 | 49.223 | 1.00 | 60.43 | O |
| ATOM | 15606 | C4 | NAG | E | 3282 | 51.144 | 24.227 | 51.060 | 1.00 | 59.24 | C |
| ATOM | 15608 | O4 | NAG | E | 3282 | 50.928 | 22.982 | 51.702 | 1.00 | 59.30 | O |
| ATOM | 15609 | C5 | NAG | E | 3282 | 50.469 | 25.441 | 51.718 | 1.00 | 57.25 | C |
| ATOM | 15611 | C6 | NAG | E | 3282 | 51.087 | 25.744 | 53.078 | 1.00 | 55.87 | C |
| ATOM | 15614 | O6 | NAG | E | 3282 | 52.436 | 26.070 | 52.833 | 1.00 | 56.35 | O |
| ATOM | 15616 | O5 | NAG | E | 3282 | 50.700 | 26.585 | 50.922 | 1.00 | 56.55 | O |
| ATOM | 15617 | C1 | MAN | E | 3283 | 52.049 | 22.569 | 52.475 | 1.00 | 60.80 | C |
| ATOM | 15619 | C2 | MAN | E | 3283 | 51.547 | 22.103 | 53.804 | 1.00 | 59.65 | C |
| ATOM | 15621 | O2 | MAN | E | 3283 | 50.445 | 21.312 | 53.451 | 1.00 | 58.83 | O |
| ATOM | 15623 | C3 | MAN | E | 3283 | 52.518 | 21.233 | 54.574 | 1.00 | 61.41 | C |
| ATOM | 15625 | O3 | MAN | E | 3283 | 51.787 | 20.219 | 55.195 | 1.00 | 63.34 | O |
| ATOM | 15626 | C4 | MAN | E | 3283 | 53.562 | 20.456 | 53.794 | 1.00 | 64.14 | C |
| ATOM | 15628 | O4 | MAN | E | 3283 | 54.592 | 20.336 | 54.757 | 1.00 | 65.32 | O |
| ATOM | 15630 | C5 | MAN | E | 3283 | 53.906 | 21.186 | 52.484 | 1.00 | 64.35 | C |
| ATOM | 15632 | C6 | MAN | E | 3283 | 54.827 | 20.559 | 51.427 | 1.00 | 66.91 | C |
| ATOM | 15635 | O6 | MAN | E | 3283 | 54.336 | 19.397 | 50.755 | 1.00 | 68.16 | O |
| ATOM | 15636 | O5 | MAN | E | 3283 | 52.651 | 21.452 | 51.873 | 1.00 | 66.18 | O |
| ATOM | 15637 | C1 | MAN | E | 3284 | 55.217 | 18.249 | 50.742 | 1.00 | 72.30 | C |
| ATOM | 15639 | C2 | MAN | E | 3284 | 55.266 | 17.684 | 52.167 | 1.00 | 74.79 | C |
| ATOM | 15641 | O2 | MAN | E | 3284 | 53.926 | 17.897 | 52.622 | 1.00 | 75.61 | O |
| ATOM | 15643 | C3 | MAN | E | 3284 | 55.505 | 16.199 | 52.355 | 1.00 | 77.19 | C |
| ATOM | 15645 | O3 | MAN | E | 3284 | 55.980 | 15.970 | 53.712 | 1.00 | 78.27 | O |
| ATOM | 15646 | C4 | MAN | E | 3284 | 54.050 | 15.999 | 51.891 | 1.00 | 74.76 | C |
| ATOM | 15648 | O4 | MAN | E | 3284 | 53.363 | 14.913 | 52.491 | 1.00 | 74.26 | O |
| ATOM | 15650 | C5 | MAN | E | 3284 | 54.093 | 16.166 | 50.311 | 1.00 | 75.13 | C |
| ATOM | 15652 | C6 | MAN | E | 3284 | 52.766 | 16.286 | 49.596 | 1.00 | 71.00 | C |
| ATOM | 15655 | O6 | MAN | E | 3284 | 51.789 | 16.197 | 50.591 | 1.00 | 67.75 | O |
| ATOM | 15656 | O5 | MAN | E | 3284 | 54.772 | 17.312 | 49.795 | 1.00 | 71.66 | O |
| ATOM | 15657 | C1 | MAN | E | 3287 | 56.557 | 14.726 | 54.281 | 1.00 | 82.57 | C |
| ATOM | 15659 | C2 | MAN | E | 3287 | 57.357 | 13.797 | 53.289 | 1.00 | 86.35 | C |
| ATOM | 15661 | O2 | MAN | E | 3287 | 58.723 | 13.344 | 53.449 | 1.00 | 90.50 | O |
| ATOM | 15662 | C3 | MAN | E | 3287 | 56.672 | 12.482 | 52.944 | 1.00 | 89.09 | C |
| ATOM | 15664 | O3 | MAN | E | 3287 | 57.659 | 11.558 | 52.507 | 1.00 | 90.57 | O |
| ATOM | 15666 | C4 | MAN | E | 3287 | 55.887 | 11.911 | 54.115 | 1.00 | 89.40 | C |
| ATOM | 15668 | O4 | MAN | E | 3287 | 55.294 | 10.689 | 53.706 | 1.00 | 92.89 | O |
| ATOM | 15670 | C5 | MAN | E | 3287 | 54.863 | 12.927 | 54.621 | 1.00 | 83.20 | C |
| ATOM | 15672 | C6 | MAN | E | 3287 | 54.074 | 12.364 | 55.791 | 1.00 | 82.64 | C |
| ATOM | 15675 | O6 | MAN | E | 3287 | 53.440 | 11.149 | 55.398 | 1.00 | 86.10 | O |
| ATOM | 15677 | O5 | MAN | E | 3287 | 55.586 | 14.035 | 55.127 | 1.00 | 82.62 | O |
| ATOM | 15678 | C1 | MAN | E | 3288 | 59.546 | 13.176 | 54.658 | 1.00 | 92.52 | C |
| ATOM | 15680 | C2 | MAN | E | 3288 | 59.349 | 14.272 | 55.676 | 1.00 | 89.65 | C |
| ATOM | 15682 | O2 | MAN | E | 3288 | 58.271 | 13.854 | 56.471 | 1.00 | 91.69 | O |
| ATOM | 15684 | C3 | MAN | E | 3288 | 60.452 | 14.356 | 56.660 | 1.00 | 90.23 | C |
| ATOM | 15686 | O3 | MAN | E | 3288 | 59.890 | 15.181 | 57.619 | 1.00 | 85.18 | O |
| ATOM | 15688 | C4 | MAN | E | 3288 | 60.741 | 13.016 | 57.331 | 1.00 | 94.39 | C |
| ATOM | 15690 | O4 | MAN | E | 3288 | 62.025 | 13.205 | 57.964 | 1.00 | 93.03 | O |
| ATOM | 15692 | C5 | MAN | E | 3288 | 60.590 | 11.833 | 56.315 | 1.00 | 97.46 | C |
| ATOM | 15694 | C6 | MAN | E | 3288 | 60.604 | 10.434 | 56.978 | 1.00 | 101.20 | C |
| ATOM | 15697 | O6 | MAN | E | 3288 | 59.305 | 9.870 | 57.154 | 1.00 | 102.55 | O |
| ATOM | 15699 | O5 | MAN | E | 3288 | 59.440 | 12.000 | 55.444 | 1.00 | 94.57 | O |
| ATOM | 15700 | C1 | MAN | E | 3285 | 52.312 | 20.696 | 57.165 | 1.00 | 71.22 | C |
| ATOM | 15702 | C2 | MAN | E | 3285 | 51.852 | 19.512 | 58.022 | 1.00 | 72.51 | C |
| ATOM | 15704 | O2 | MAN | E | 3285 | 52.078 | 19.647 | 59.410 | 1.00 | 75.71 | O |
| ATOM | 15705 | C3 | MAN | E | 3285 | 50.367 | 19.348 | 57.867 | 1.00 | 68.59 | C |
| ATOM | 15707 | O3 | MAN | E | 3285 | 50.001 | 18.467 | 58.884 | 1.00 | 72.03 | O |
| ATOM | 15709 | C4 | MAN | E | 3285 | 49.651 | 20.659 | 58.121 | 1.00 | 64.77 | C |
| ATOM | 15711 | O4 | MAN | E | 3285 | 48.263 | 20.485 | 57.943 | 1.00 | 65.10 | O |
| ATOM | 15713 | C5 | MAN | E | 3285 | 50.145 | 21.664 | 57.134 | 1.00 | 62.44 | C |
| ATOM | 15715 | C6 | MAN | E | 3285 | 49.441 | 22.972 | 57.332 | 1.00 | 57.64 | C |
| ATOM | 15718 | O6 | MAN | E | 3285 | 49.509 | 23.586 | 56.085 | 1.00 | 54.72 | O |
| ATOM | 15720 | O5 | MAN | E | 3285 | 51.537 | 21.875 | 57.328 | 1.00 | 68.41 | O |
| ATOM | 15721 | C1 | MAN | E | 3286 | 52.667 | 20.955 | 59.763 | 1.00 | 77.35 | C |
| ATOM | 15723 | C2 | MAN | E | 3286 | 52.629 | 21.164 | 61.302 | 1.00 | 76.62 | C |
| ATOM | 15725 | O2 | MAN | E | 3286 | 52.926 | 19.916 | 61.946 | 1.00 | 81.48 | O |
| ATOM | 15727 | C3 | MAN | E | 3286 | 53.567 | 22.287 | 61.783 | 1.00 | 73.66 | C |
| ATOM | 15729 | O3 | MAN | E | 3286 | 54.478 | 21.668 | 62.617 | 1.00 | 73.03 | O |
| ATOM | 15731 | C4 | MAN | E | 3286 | 54.473 | 23.006 | 60.754 | 1.00 | 75.14 | C |
| ATOM | 15733 | O4 | MAN | E | 3286 | 54.449 | 24.416 | 61.021 | 1.00 | 71.56 | O |
| ATOM | 15735 | C5 | MAN | E | 3286 | 54.173 | 22.670 | 59.260 | 1.00 | 76.43 | C |
| ATOM | 15737 | C6 | MAN | E | 3286 | 55.208 | 23.365 | 58.290 | 1.00 | 76.78 | C |
| ATOM | 15740 | O6 | MAN | E | 3286 | 55.328 | 23.003 | 56.888 | 1.00 | 76.61 | O |
| ATOM | 15742 | O5 | MAN | E | 3286 | 54.002 | 21.227 | 59.234 | 1.00 | 77.45 | O |
| ATOM | 15743 | C1 | MAN | E | 3289 | 50.258 | 15.461 | 49.783 | 1.00 | 75.63 | C |
| ATOM | 15745 | C2 | MAN | E | 3289 | 50.034 | 14.267 | 48.872 | 1.00 | 75.77 | C |
| ATOM | 15747 | O2 | MAN | E | 3289 | 48.660 | 14.201 | 48.764 | 1.00 | 72.18 | O |
| ATOM | 15749 | C3 | MAN | E | 3289 | 50.526 | 14.452 | 47.437 | 1.00 | 78.29 | C |
| ATOM | 15751 | O3 | MAN | E | 3289 | 49.933 | 13.510 | 46.572 | 1.00 | 79.15 | O |
| ATOM | 15753 | C4 | MAN | E | 3289 | 50.081 | 15.789 | 46.882 | 1.00 | 77.40 | C |
| ATOM | 15755 | O4 | MAN | E | 3289 | 50.521 | 15.836 | 45.541 | 1.00 | 77.75 | O |
| ATOM | 15757 | C5 | MAN | E | 3289 | 50.659 | 16.920 | 47.772 | 1.00 | 76.35 | C |
| ATOM | 15759 | C6 | MAN | E | 3289 | 50.230 | 18.330 | 47.313 | 1.00 | 71.38 | C |
| ATOM | 15762 | O6 | MAN | E | 3289 | 51.375 | 19.128 | 47.137 | 1.00 | 68.05 | O |
| ATOM | 15764 | O5 | MAN | E | 3289 | 50.198 | 16.752 | 49.136 | 1.00 | 76.70 | O |
| ATOM | 15765 | C1 | NAG | E | 3371 | 38.544 | 32.137 | 31.287 | 1.00 | 73.74 | C |
| ATOM | 15768 | C2 | NAG | E | 3371 | 39.910 | 31.524 | 30.912 | 1.00 | 72.50 | C |
| ATOM | 15770 | N2 | NAG | E | 3371 | 40.864 | 31.546 | 32.013 | 1.00 | 71.87 | N |
| ATOM | 15772 | C7 | NAG | E | 3371 | 41.239 | 30.472 | 32.757 | 1.00 | 70.84 | C |
| ATOM | 15773 | O7 | NAG | E | 3371 | 40.476 | 29.555 | 33.102 | 1.00 | 70.45 | O |
| ATOM | 15774 | C8 | NAG | E | 3371 | 42.672 | 30.426 | 33.238 | 1.00 | 70.39 | C |
| ATOM | 15778 | C3 | NAG | E | 3371 | 40.656 | 32.193 | 29.783 | 1.00 | 75.42 | C |
| ATOM | 15780 | O3 | NAG | E | 3371 | 41.791 | 31.359 | 29.440 | 1.00 | 77.30 | O |
| ATOM | 15782 | C4 | NAG | E | 3371 | 39.730 | 32.463 | 28.602 | 1.00 | 76.98 | C |
| ATOM | 15784 | O4 | NAG | E | 3371 | 40.419 | 33.182 | 27.568 | 1.00 | 80.73 | O |
| ATOM | 15785 | C5 | NAG | E | 3371 | 38.541 | 33.295 | 29.072 | 1.00 | 77.51 | C |
| ATOM | 15787 | C6 | NAG | E | 3371 | 37.559 | 33.426 | 27.908 | 1.00 | 79.31 | C |
| ATOM | 15790 | O6 | NAG | E | 3371 | 36.679 | 34.496 | 28.177 | 1.00 | 80.38 | O |
| ATOM | 15792 | O5 | NAG | E | 3371 | 37.813 | 32.803 | 30.226 | 1.00 | 77.42 | O |
| ATOM | 15793 | C1 | NAG | E | 3372 | 41.583 | 32.516 | 26.923 | 1.00 | 81.06 | C |
| ATOM | 15795 | C2 | NAG | E | 3372 | 42.328 | 33.443 | 25.948 | 1.00 | 82.94 | C |
| ATOM | 15797 | N2 | NAG | E | 3372 | 41.740 | 34.767 | 25.838 | 1.00 | 82.97 | N |
| ATOM | 15799 | C7 | NAG | E | 3372 | 42.233 | 35.902 | 26.335 | 1.00 | 82.90 | C |
| ATOM | 15800 | O7 | NAG | E | 3372 | 41.520 | 36.914 | 26.248 | 1.00 | 82.64 | O |
| ATOM | 15801 | C8 | NAG | E | 3372 | 43.607 | 35.961 | 26.991 | 1.00 | 82.07 | C |
| ATOM | 15805 | C3 | NAG | E | 3372 | 42.419 | 32.829 | 24.547 | 1.00 | 85.35 | C |
| ATOM | 15807 | O3 | NAG | E | 3372 | 43.207 | 33.591 | 23.635 | 1.00 | 87.85 | O |
| ATOM | 15809 | C4 | NAG | E | 3372 | 43.054 | 31.472 | 24.780 | 1.00 | 84.91 | C |
| ATOM | 15811 | O4 | NAG | E | 3372 | 43.723 | 30.920 | 23.637 | 1.00 | 91.00 | O |
| ATOM | 15812 | C5 | NAG | E | 3372 | 41.959 | 30.586 | 25.314 | 1.00 | 82.11 | C |
| ATOM | 15814 | C6 | NAG | E | 3372 | 42.432 | 29.260 | 25.924 | 1.00 | 80.32 | C |
| ATOM | 15817 | O6 | NAG | E | 3372 | 43.839 | 29.217 | 26.026 | 1.00 | 82.02 | O |
| ATOM | 15819 | O5 | NAG | E | 3372 | 41.168 | 31.317 | 26.251 | 1.00 | 83.28 | O |
| ATOM | 15820 | C1 | MAN | E | 3373 | 43.193 | 31.163 | 22.297 | 1.00 | 90.26 | C |
| ATOM | 15822 | C2 | MAN | E | 3373 | 43.289 | 29.790 | 21.572 | 1.00 | 89.29 | C |
| ATOM | 15824 | O2 | MAN | E | 3373 | 42.106 | 29.541 | 20.822 | 1.00 | 88.30 | O |
| ATOM | 15826 | C3 | MAN | E | 3373 | 44.594 | 29.551 | 20.798 | 1.00 | 90.57 | C |
| ATOM | 15828 | O3 | MAN | E | 3373 | 44.458 | 28.528 | 19.795 | 1.00 | 92.13 | O |
| ATOM | 15829 | C4 | MAN | E | 3373 | 45.145 | 30.855 | 20.206 | 1.00 | 93.50 | C |
| ATOM | 15831 | O4 | MAN | E | 3373 | 46.449 | 30.566 | 19.675 | 1.00 | 97.00 | O |
| ATOM | 15833 | C5 | MAN | E | 3373 | 45.156 | 32.027 | 21.232 | 1.00 | 91.21 | C |
| ATOM | 15835 | C6 | MAN | E | 3373 | 45.845 | 33.348 | 20.765 | 1.00 | 92.87 | C |
| ATOM | 15838 | O6 | MAN | E | 3373 | 45.180 | 34.027 | 19.709 | 1.00 | 91.50 | O |
| ATOM | 15840 | O5 | MAN | E | 3373 | 43.831 | 32.276 | 21.657 | 1.00 | 90.06 | O |
| ATOM | 15841 | C1 | MAN | E | 3374 | 45.299 | 27.327 | 19.975 | 1.00 | 91.45 | C |
| ATOM | 15843 | C2 | MAN | E | 3374 | 44.665 | 25.926 | 19.573 | 1.00 | 90.43 | C |
| ATOM | 15845 | O2 | MAN | E | 3374 | 43.307 | 25.675 | 19.923 | 1.00 | 85.56 | O |
| ATOM | 15847 | C3 | MAN | E | 3374 | 44.958 | 25.484 | 18.106 | 1.00 | 93.36 | C |
| ATOM | 15849 | O3 | MAN | E | 3374 | 43.909 | 25.879 | 17.264 | 1.00 | 92.46 | O |
| ATOM | 15851 | C4 | MAN | E | 3374 | 46.346 | 25.944 | 17.578 | 1.00 | 95.44 | C |
| ATOM | 15853 | O4 | MAN | E | 3374 | 47.038 | 24.929 | 16.892 | 1.00 | 96.98 | O |
| ATOM | 15855 | C5 | MAN | E | 3374 | 47.209 | 26.363 | 18.774 | 1.00 | 94.63 | C |
| ATOM | 15857 | C6 | MAN | E | 3374 | 48.726 | 26.471 | 18.535 | 1.00 | 95.51 | C |
| ATOM | 15860 | O6 | MAN | E | 3374 | 49.041 | 26.392 | 17.176 | 1.00 | 99.02 | O |
| ATOM | 15862 | O5 | MAN | E | 3374 | 46.576 | 27.526 | 19.340 | 1.00 | 94.07 | O |
| ATOM | 15863 | C1 | NAG | E | 3891 | 45.808 | 53.754 | 43.706 | 1.00 | 74.28 | C |
| ATOM | 15866 | C2 | NAG | E | 3891 | 45.380 | 54.293 | 42.335 | 1.00 | 76.79 | C |
| ATOM | 15868 | N2 | NAG | E | 3891 | 43.931 | 54.439 | 42.244 | 1.00 | 77.53 | N |
| ATOM | 15870 | C7 | NAG | E | 3891 | 43.082 | 53.652 | 41.542 | 1.00 | 79.46 | C |
| ATOM | 15871 | O7 | NAG | E | 3891 | 42.117 | 54.113 | 40.922 | 1.00 | 80.77 | O |
| ATOM | 15872 | C8 | NAG | E | 3891 | 43.269 | 52.147 | 41.487 | 1.00 | 77.83 | C |
| ATOM | 15876 | C3 | NAG | E | 3891 | 45.958 | 53.518 | 41.171 | 1.00 | 79.74 | C |
| ATOM | 15878 | O3 | NAG | E | 3891 | 45.933 | 54.287 | 39.941 | 1.00 | 85.15 | O |
| ATOM | 15880 | C4 | NAG | E | 3891 | 47.366 | 53.013 | 41.512 | 1.00 | 79.56 | C |
| ATOM | 15882 | O4 | NAG | E | 3891 | 47.766 | 52.108 | 40.515 | 1.00 | 81.56 | O |
| ATOM | 15884 | C5 | NAG | E | 3891 | 47.547 | 52.327 | 42.862 | 1.00 | 74.77 | C |
| ATOM | 15886 | C6 | NAG | E | 3891 | 49.038 | 51.935 | 43.162 | 1.00 | 76.89 | C |
| ATOM | 15889 | O6 | NAG | E | 3891 | 50.128 | 52.559 | 42.436 | 1.00 | 78.59 | O |
| ATOM | 15891 | O5 | NAG | E | 3891 | 47.051 | 53.133 | 43.897 | 1.00 | 71.16 | O |
| ATOM | 15892 | C1 | NAG | E | 3892 | 49.560 | 50.560 | 39.673 | 1.00 | 85.54 | C |
| ATOM | 15895 | C2 | NAG | E | 3892 | 51.079 | 50.372 | 39.365 | 1.00 | 87.01 | C |
| ATOM | 15897 | N2 | NAG | E | 3892 | 51.830 | 49.743 | 40.498 | 1.00 | 86.01 | N |
| ATOM | 15899 | C7 | NAG | E | 3892 | 53.058 | 50.168 | 41.010 | 1.00 | 88.55 | C |
| ATOM | 15900 | O7 | NAG | E | 3892 | 54.106 | 50.400 | 40.348 | 1.00 | 92.23 | O |
| ATOM | 15901 | C8 | NAG | E | 3892 | 53.194 | 50.394 | 42.509 | 1.00 | 85.72 | C |
| ATOM | 15905 | C3 | NAG | E | 3892 | 51.752 | 51.651 | 38.791 | 1.00 | 89.93 | C |
| ATOM | 15907 | O3 | NAG | E | 3892 | 53.012 | 51.322 | 38.204 | 1.00 | 93.75 | O |
| ATOM | 15909 | C4 | NAG | E | 3892 | 50.816 | 52.383 | 37.790 | 1.00 | 92.19 | C |
| ATOM | 15911 | O4 | NAG | E | 3892 | 51.469 | 53.394 | 36.980 | 1.00 | 97.04 | O |
| ATOM | 15913 | C5 | NAG | E | 3892 | 49.621 | 52.861 | 38.652 | 1.00 | 90.09 | C |
| ATOM | 15915 | C6 | NAG | E | 3892 | 48.688 | 53.957 | 38.073 | 1.00 | 91.72 | C |
| ATOM | 15918 | O6 | NAG | E | 3892 | 49.232 | 54.724 | 36.977 | 1.00 | 93.72 | O |
| ATOM | 15920 | O5 | NAG | E | 3892 | 48.877 | 51.689 | 39.028 | 1.00 | 88.11 | O |
| ATOM | 15921 | C1 | NAG | E | 4201 | 42.244 | 56.852 | 52.671 | 1.00 | 63.81 | C |
| ATOM | 15924 | C2 | NAG | E | 4201 | 43.345 | 57.207 | 51.711 | 1.00 | 65.17 | C |
| ATOM | 15926 | N2 | NAG | E | 4201 | 44.591 | 56.697 | 52.209 | 1.00 | 64.00 | N |
| ATOM | 15928 | C7 | NAG | E | 4201 | 45.518 | 56.163 | 51.423 | 1.00 | 64.41 | C |
| ATOM | 15929 | O7 | NAG | E | 4201 | 45.413 | 56.030 | 50.212 | 1.00 | 65.04 | O |
| ATOM | 15930 | C8 | NAG | E | 4201 | 46.795 | 55.727 | 52.065 | 1.00 | 64.60 | C |
| ATOM | 15934 | C3 | NAG | E | 4201 | 43.381 | 58.699 | 51.617 | 1.00 | 68.66 | C |
| ATOM | 15936 | O3 | NAG | E | 4201 | 44.262 | 58.910 | 50.571 | 1.00 | 74.46 | O |
| ATOM | 15938 | C4 | NAG | E | 4201 | 42.044 | 59.335 | 51.220 | 1.00 | 71.76 | C |
| ATOM | 15940 | O4 | NAG | E | 4201 | 41.985 | 60.760 | 51.475 | 1.00 | 76.53 | O |
| ATOM | 15941 | C5 | NAG | E | 4201 | 40.915 | 58.632 | 51.980 | 1.00 | 68.94 | C |
| ATOM | 15943 | C6 | NAG | E | 4201 | 39.614 | 58.830 | 51.256 | 1.00 | 70.71 | C |
| ATOM | 15946 | O6 | NAG | E | 4201 | 38.706 | 58.151 | 52.076 | 1.00 | 70.23 | O |
| ATOM | 15948 | O5 | NAG | E | 4201 | 41.037 | 57.234 | 52.120 | 1.00 | 65.06 | O |
| ATOM | 15949 | C1 | NAG | E | 4202 | 42.056 | 61.718 | 50.372 | 1.00 | 81.14 | C |
| ATOM | 15951 | C2 | NAG | E | 4202 | 41.295 | 63.019 | 50.736 | 1.00 | 83.23 | C |
| ATOM | 15953 | N2 | NAG | E | 4202 | 40.158 | 62.736 | 51.611 | 1.00 | 81.14 | N |
| ATOM | 15955 | C7 | NAG | E | 4202 | 40.195 | 62.977 | 52.938 | 1.00 | 81.55 | C |
| ATOM | 15956 | O7 | NAG | E | 4202 | 40.397 | 62.106 | 53.790 | 1.00 | 75.71 | O |
| ATOM | 15957 | C8 | NAG | E | 4202 | 39.997 | 64.413 | 53.403 | 1.00 | 84.96 | C |
| ATOM | 15961 | C3 | NAG | E | 4202 | 40.871 | 63.865 | 49.497 | 1.00 | 87.52 | C |
| ATOM | 15963 | O3 | NAG | E | 4202 | 40.860 | 65.241 | 49.801 | 1.00 | 91.09 | O |
| ATOM | 15965 | C4 | NAG | E | 4202 | 41.751 | 63.571 | 48.257 | 1.00 | 88.70 | C |
| ATOM | 15967 | O4 | NAG | E | 4202 | 41.562 | 64.296 | 46.993 | 1.00 | 92.75 | O |
| ATOM | 15968 | C5 | NAG | E | 4202 | 41.445 | 62.067 | 48.006 | 1.00 | 86.08 | C |
| ATOM | 15970 | C6 | NAG | E | 4202 | 42.113 | 61.443 | 46.760 | 1.00 | 86.81 | C |
| ATOM | 15973 | O6 | NAG | E | 4202 | 43.309 | 60.781 | 47.074 | 1.00 | 86.56 | O |
| ATOM | 15975 | O5 | NAG | E | 4202 | 41.555 | 61.202 | 49.146 | 1.00 | 82.49 | O |
| ATOM | 15976 | C1 | MAN | E | 4203 | 41.597 | 65.753 | 46.758 | 1.00 | 96.52 | C |
| ATOM | 15978 | C2 | MAN | E | 4203 | 42.878 | 66.374 | 46.086 | 1.00 | 98.52 | C |
| ATOM | 15980 | O2 | MAN | E | 4203 | 43.726 | 65.559 | 45.284 | 1.00 | 94.95 | O |
| ATOM | 15982 | C3 | MAN | E | 4203 | 42.475 | 67.691 | 45.329 | 1.00 | 104.12 | C |
| ATOM | 15984 | O3 | MAN | E | 4203 | 42.359 | 67.581 | 43.900 | 1.00 | 106.03 | O |
| ATOM | 15986 | C4 | MAN | E | 4203 | 41.185 | 68.311 | 45.942 | 1.00 | 105.60 | C |
| ATOM | 15988 | O4 | MAN | E | 4203 | 41.065 | 69.698 | 45.630 | 1.00 | 108.12 | O |
| ATOM | 15990 | C5 | MAN | E | 4203 | 41.097 | 68.015 | 47.482 | 1.00 | 102.96 | C |
| ATOM | 15992 | C6 | MAN | E | 4203 | 39.839 | 68.676 | 48.110 | 1.00 | 104.13 | C |
| ATOM | 15995 | O6 | MAN | E | 4203 | 39.443 | 67.969 | 49.283 | 1.00 | 100.82 | O |
| ATOM | 15997 | O5 | MAN | E | 4203 | 41.313 | 66.623 | 47.864 | 1.00 | 97.25 | O |
| ATOM | 15998 | C1 | NAG | E | 5041 | 12.333 | 62.275 | 39.325 | 1.00 | 137.11 | C |
| ATOM | 16001 | C2 | NAG | E | 5041 | 12.986 | 63.604 | 39.616 | 1.00 | 138.53 | C |
| ATOM | 16003 | N2 | NAG | E | 5041 | 13.364 | 63.660 | 41.044 | 1.00 | 136.10 | N |
| ATOM | 16005 | C7 | NAG | E | 5041 | 14.433 | 63.080 | 41.645 | 1.00 | 128.39 | C |
| ATOM | 16006 | O7 | NAG | E | 5041 | 14.366 | 62.041 | 42.320 | 1.00 | 122.17 | O |
| ATOM | 16007 | C8 | NAG | E | 5041 | 15.723 | 63.845 | 41.521 | 1.00 | 127.26 | C |
| ATOM | 16011 | C3 | NAG | E | 5041 | 12.054 | 64.733 | 39.116 | 1.00 | 144.44 | C |
| ATOM | 16013 | O3 | NAG | E | 5041 | 12.354 | 64.855 | 37.737 | 1.00 | 145.80 | O |
| ATOM | 16015 | C4 | NAG | E | 5041 | 10.515 | 64.539 | 39.222 | 1.00 | 148.10 | C |
| ATOM | 16017 | O4 | NAG | E | 5041 | 9.929 | 65.156 | 40.370 | 1.00 | 149.99 | O |
| ATOM | 16019 | C5 | NAG | E | 5041 | 10.130 | 63.058 | 39.106 | 1.00 | 144.50 | C |
| ATOM | 16021 | C6 | NAG | E | 5041 | 8.913 | 62.764 | 38.230 | 1.00 | 147.36 | C |
| ATOM | 16024 | O6 | NAG | E | 5041 | 7.864 | 63.649 | 38.544 | 1.00 | 153.22 | O |
| ATOM | 16026 | O5 | NAG | E | 5041 | 11.210 | 62.427 | 38.472 | 1.00 | 141.22 | O |
| ATOM | 16027 | C1 | NAG | E | 5441 | 0.234 | 62.127 | 19.661 | 1.00 | 208.01 | C |
| ATOM | 16030 | C2 | NAG | E | 5441 | −0.771 | 60.962 | 19.723 | 1.00 | 206.40 | C |
| ATOM | 16032 | N2 | NAG | E | 5441 | −1.608 | 60.874 | 18.514 | 1.00 | 211.28 | N |
| ATOM | 16034 | C7 | NAG | E | 5441 | −2.489 | 59.886 | 18.295 | 1.00 | 210.66 | C |
| ATOM | 16035 | O7 | NAG | E | 5441 | −2.188 | 58.694 | 18.394 | 1.00 | 203.38 | O |
| ATOM | 16036 | C8 | NAG | E | 5441 | −3.897 | 60.288 | 17.910 | 1.00 | 217.87 | C |
| ATOM | 16040 | C3 | NAG | E | 5441 | −1.619 | 61.139 | 20.977 | 1.00 | 207.06 | C |
| ATOM | 16042 | O3 | NAG | E | 5441 | −0.826 | 60.941 | 22.134 | 1.00 | 198.40 | O |
| ATOM | 16044 | C4 | NAG | E | 5441 | −2.284 | 62.530 | 20.945 | 1.00 | 213.89 | C |
| ATOM | 16046 | O4 | NAG | E | 5441 | −3.220 | 62.583 | 19.875 | 1.00 | 218.05 | O |
| ATOM | 16048 | C5 | NAG | E | 5441 | −1.247 | 63.664 | 20.825 | 1.00 | 214.53 | C |
| ATOM | 16050 | C6 | NAG | E | 5441 | −1.248 | 64.554 | 22.067 | 1.00 | 214.73 | C |
| ATOM | 16053 | O6 | NAG | E | 5441 | −2.440 | 65.317 | 22.081 | 1.00 | 221.76 | O |
| ATOM | 16055 | O5 | NAG | E | 5441 | 0.074 | 63.184 | 20.624 | 1.00 | 209.23 | O |
| ATOM | 16056 | C1 | NAG | E | 5791 | 8.755 | 20.579 | −4.659 | 1.00 | 160.24 | C |
| ATOM | 16059 | C2 | NAG | E | 5791 | 10.058 | 20.624 | −5.492 | 1.00 | 160.36 | C |
| ATOM | 16061 | N2 | NAG | E | 5791 | 10.855 | 21.828 | −5.207 | 1.00 | 160.00 | N |
| ATOM | 16063 | C7 | NAG | E | 5791 | 12.120 | 21.841 | −4.740 | 1.00 | 156.65 | C |
| ATOM | 16064 | O7 | NAG | E | 5791 | 12.480 | 22.620 | −3.870 | 1.00 | 154.63 | O |
| ATOM | 16065 | C8 | NAG | E | 5791 | 13.175 | 20.917 | −5.291 | 1.00 | 156.27 | C |
| ATOM | 16069 | C3 | NAG | E | 5791 | 9.820 | 20.532 | −7.008 | 1.00 | 164.24 | C |
| ATOM | 16071 | O3 | NAG | E | 5791 | 11.069 | 20.293 | −7.622 | 1.00 | 162.89 | O |
| ATOM | 16073 | C4 | NAG | E | 5791 | 8.760 | 19.484 | −7.413 | 1.00 | 166.62 | C |
| ATOM | 16075 | O4 | NAG | E | 5791 | 8.408 | 19.503 | −8.803 | 1.00 | 169.76 | O |
| ATOM | 16077 | C5 | NAG | E | 5791 | 7.504 | 19.627 | −6.540 | 1.00 | 165.87 | C |
| ATOM | 16079 | C6 | NAG | E | 5791 | 6.460 | 18.548 | −6.894 | 1.00 | 167.55 | C |
| ATOM | 16082 | O6 | NAG | E | 5791 | 6.356 | 18.315 | −8.298 | 1.00 | 169.02 | O |
| ATOM | 16084 | O5 | NAG | E | 5791 | 7.861 | 19.573 | −5.156 | 1.00 | 161.26 | O |
| END | | | | | | | | | | | |

### REFERENCES

1. Ferguson, K. M., Darling, P. J., Mohan, M. J., Macatee, T. L., and Lemmon, M. A. (2000). Extracellular domains drive homo- but not hetero-dimerization of erbB receptors. Embo J 19, 4632-4643.
2. Otwinowski, Z., and Minor, W. (1997). Processing of X-ray Diffraction Data Collected in Oscillation Mode. In Macromolecular Crystallography, Volume 276, C. W. Carter Jr. and R. M. Sweet, eds. (New York: Academic Press), pp. 307-326.
3. CCP4 (1994). The CCP4 Suite: Programs for Protein-Crystallography. Acta Cryst. D50, 760-763.
4. Jones, T. A., Zou, J. Y., Cowan, S. W., and Kjeldgaard (1991). Improved methods for building protein models in electron density maps and the location of errors in these models. Acta Crystallogr A 47 (Pt 2), 110-119.
5. Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998). Crystallography & NMR system: A new software suite for macromolecular structure determination. Acta Crystallogr D Biol Crystallogr 54 (Pt 5), 905-921.
6. Winn, M. D., Isupov, M. N., and Murshudov, G. N. (2001). Use of TLS parameters to model anisotropic displacements in macromolecular refinement. Acta Crystallogr D Biol Crystallogr 57, 122-133.
7. Garrett, T. P., McKern, N. M., Lou, M., Elleman, T. C., Adams, T. E., Lovrecz, G. O., Zhu, H. J., Walker, F., Frenkel, M. J., Hoyne, P. A., Jorissen, R. N., Nice, E. C., Burgess, A. W., and Ward, C. W. (2002). Crystal structure of a truncated epidermal growth factor receptor extracellular domain bound to transforming growth factor alpha. Cell 110, 763-773.
8. Ogiso, H., Ishitani, R., Nureki, O., Fukai, S., Yamanaka, M., Kim, J. H., Saito, K., Sakamoto, A., Inoue, M., Shirouzu, M., and Yokoyama, S. (2002). Crystal structure of the complex of human epidermal growth factor and receptor extracellular domains. Cell 110, 775-787.
9. Ferguson, K. M., Berger, M. B., Mendrola, J. M., Cho, H. S., Leahy, D. J., and Lemmon, M. A. (2003). EGF activates its receptor by removing interactions that autoinhibit ectodomain dimerization. Mol Cell 11, 507-517.
10. Jorgensen W. L., (2004). The many roles of computation on drug discovery. Science 303, 1813-1818.

What is claimed:

1. A crystal of a receptor-antibody complex comprising a receptor-antibody complex of an epidermal growth factor receptor (EGFR) extracellular domain and cetuximab Fab, wherein the crystal has a resolution determined by X-ray crystallography of better than about 5.0 Angstroms.

2. The crystal of claim 1, wherein the crystal has a resolution determined by X-ray crystallography of better than about 4.0 Angstroms.

3. The crystal of claim 2, wherein the crystal has a resolution determined by X-ray crystallography of better than about 3.0 Angstroms.

4. The crystal of claim 1, wherein the crystal belongs to space group P2₁ and has unit cell dimensions a=77.8 Å, b=70.9 Å, c=147.1 Å, and β=102.5°.

5. The crystal of claim 1, having atomic coordinates provided in Table 2.

6. A method for preparing a crystal of a complex of an epidermal growth factor receptor (EGFR) extracellular domain and cetuximab Fab comprising preparing a solution containing the extracellular domain of EGFR and cetuximab Fab fragment, and growing the crystal.

7. The method of claim 6, wherein the pH of the solution is about 6.0 to about 8.0.

8. A method of identifying a mimetic of cetuximab comprising comparing a three-dimensional structure of the mimetic with a three-dimensional structure determined for the complex of claim 1.

9. The method of claim 8, wherein the three dimensional structure of the mimetic is compared with at least a subset of the coordinates provided in Table 2.

10. The method of claim 8, wherein identifying a mimetic is carried out by comparing the three-dimensional structure of the mimetic against the coordinates of at least one EGFR amino acid bound by cetuximab Fab.

11. The method of claim 9, wherein the EGFR amino acid is selected from the group consisting of Gln 384, Gln 408, Ser 418, Ser 440, Lys 465, Ser 468, and Asn 469.

12. The method of claim 8, wherein the locations of atoms of the mimetic that contact EGFR correspond to atoms of cetuximab that contact EGFR.

13. The method of claim 8, wherein identifying a mimetic comprises comparing a three dimensional structure of a mimetic with the atomic coordinates of a region of EGFR selected from the group consisting of about amino acid residue 350 to about amino acid residue 354, about amino acid residue 380 to about amino acid residue 385, about amino acid residue 405 to about amino acid residue 420, about amino acid residue 435 to about amino acid residue 475 and combinations thereof.

14. The method of claim 8, wherein the mimetic is a small molecule.

15. The method of claim 8, wherein the mimetic is a peptide.

16. The method of claim [0014], wherein the peptide is an antibody or a fragment thereof.

17. The method of claim 8, wherein the method is carried out with use of a computer.

18. The method of claim 8, further comprising synthesizing the mimetic and assaying its binding or physiological activity.

19. The method of claim [0017], wherein the mimetic binds to EGFR with similar affinity as cetuximab Fab.

20. The method of claim [0017], wherein the mimetic inhibits dimerization of EGFR expressed by a cell.

21. The method of claim [0017], wherein the mimetic inhibits tyrosine kinase activity of the receptor.

22. The method of claim [0017], wherein the mimetic blocks binding of EGF to EGFR.

23. A method for identifying a mimetic of cetuximab, comprising:
(a) introducing in silico substitutions in at least a single CDR region of cetuximab to obtain a pool of variants; and
(b) using a computer and at least a subset of the EGFR coordinates provided in Table 2 to select a variant with improved EGFR binding characteristics.

24. The method of claim 23, further comprising determining the biological activity of the mimetic.

25. The method of claim 23, wherein at most a single substitution is made in each CDR.

26. The method of claim 23, wherein substitutions are made solely in a CDR3 region.

27. A computer-assisted method for identifying a potential antagonist mimetic that binds the extracellular domain of EGFR comprising a processor, a data storage system, an input device, and an output device, comprising:
(a) inputting into the programmed computer through said input device data comprising the three-dimensional coordinates of a subset of the atoms of EGFR as set out in Table 2;
(a) providing a database of chemical and peptide structures stored in said computer data storage system;
(b) selecting from said database, using computer methods, structures having a portion that is structurally similar to said criteria data set; and
(b) outputting to said output device the selected chemical structures having a portion similar to said criteria data set.

28. A machine-readable medium having stored thereon a plurality of executable instructions to perform a method to identify a mimetic of cetuximab using a crystal of a receptor-antibody complex comprising a receptor-antibody complex of an epidermal growth factor receptor (EGFR) extracellular domain and cetuximab Fab, the method comprising:
comparing a three-dimensional structure of a mimetic with a three dimensional structure an epidermal growth factor receptor (EGFR) extracellular domain and cetuximab Fab having an X-ray crystallography resolution of better than about 5.0 Angstroms.

29. The machine-readable medium of claim [0019], wherein the EGFR coordinates comprise at least a subset of the atomic coordinates of Table 2.

30. The machine-readable medium of claim [0019], wherein the three-dimensional structure of the mimetic is compared with at least a subset of the atomic coordinates of Table 2.

31. The machine-readable medium of claim [0019], wherein identifying a mimetic comprises comparing the three-dimensional structure of a mimetic with a three-dimensional structure of at least one EGFR amino acid bound by cetuximab Fab.

32. The machine-readable medium of claim [0019], wherein identifying a mimetic comprises comparing a three dimensional structure of a mimetic with the atomic coordinates of a region of EGFR selected from the group consisting of about amino acid residue 350 to about amino acid residue 354, about amino acid residue 380 to about amino acid residue 385, about amino acid residue 405 to about amino acid residue 420, about amino acid residue 435 to about amino acid residue 475 and combinations thereof.

33. A machine-readable medium having stored thereon a plurality of executable instructions to perform a method for identifying a mimetic of cetuximab, the method comprising:
(a) introducing in silico substitutions in at least a single CDR region of cetuximab to obtain a pool of variants; and
(b) using a computer and at least a subset of the EGFR coordinates provided in Table 2 to select a variant with improved EGFR binding characteristics.

34. A cetuximab mimetic identified by the method of any one of claims 8 to 27.

35. A method of inhibiting EGFR comprising administering a mimetic of claim [0022].

36. A method of inhibiting tumor growth in a mammal comprising administering a therapeutically effective amount of a cetuximab mimetic of claim [0022].

37. The method of claim 36, wherein the tumor expresses EGFR.

38. The method of claim 36, wherein the tumor overexpresses EGFR.

39. The method of claim 36, wherein the tumor is a primary tumor.

40. The method of claim 36, wherein the tumor is a metastatic tumor.

41. The method of claim 36, wherein the tumor is a refractory tumor.

42. The method of claim 36, wherein the tumor is a vascularized tumor.

43. The method of claim 36, wherein the tumor is selected from the group consisting of a colorectal tumor, a head and neck tumor, a pancreatic tumor, a lung tumor, a breast tumor, a renal cell carcinoma, and a glioblastoma.

44. The method of claim 36, wherein the cetuximab mimetic is administered in combination with an anti-neoplastic agent.

45. The method of claim 44, wherein the antineoplastic agent is a chemotherapeutic agent.

46. The method of claim 44, wherein the antineoplastic agent is irinotecan (CPT-11).

47. The method of claim 44, wherein the antineoplastic agent is radiation.

48. The method of claim 36, wherein the cetuximab mimetic is administered in combination with an EGFR antagonist.

49. The method of claim 48, wherein the EGFR antagonist is an intracellular EGFR antagonist.

50. The method of claim 36, wherein the cetuximab mimetic is administered in combination with a VEGFR antagonist.

51. The method of claim 36, wherein the cetuximab mimetic is administered in combination with an insulin like growth factor receptor (IGFR) antagonist.

52. A method of treating a hyperproliferative disease comprising administering a therapeutically effective amount of a cetuximab mimetic of claim [0022].

53. The method of claim 52, wherein the hyperproliferative disease is psoriasis.

54. The method of claim 52, wherein the cetuximab mimetic is administered in combination with a topical or systemic agent for psoriasis.

55. The method of claim 52, wherein the cetuximab mimetic is administered in combination with a corticosteroid.

56. The method of claim 52, wherein the cetuximab mimetic is administered in combination with a retinoid.
